(19) United States
(12) Patent Application Publication
(10) Pub. No.: US 2026/0062407 A1
(43) Pub. Date: Mar. 5, 2026

(54) PEROXIREDOXIN 3 INHIBITORS AND METHODS OF USE FOR TREATING CANCER

(71) Applicant: WAKE FOREST UNIVERSITY HEALTH SCIENCES, Winston-Salem, NC (US)

(72) Inventors: W. Todd LOWTHER, Pfafftown, NC (US); Terrence L. SMALLEY, Greensboro, NC (US); Kimberly J. NELSON, Clemmons, NC (US)

(21) Appl. No.: 19/106,359

(22) PCT Filed: Aug. 25, 2023
(86) PCT No.: PCT/US2023/072904

§ 371 Date: Feb. 25, 2025

Related U.S. Application Data
(60) Provisional application No. 63/373,626, filed on Aug. 26, 2022.

(51) Int. Cl. C07D 417/04; A61K 31/282; A61K 31/337; A61K 31/426; A61K 31/427; A61K 31/4439; A61K 31/454; A61K 31/496; A61K 31/5377; A61K 31/704; A61K 38/05; A61P 35/00; C07D 277/56; C07D 417/12; C07D 417/14

(52) U.S. Cl. CPC C07D 417/04; A61K 31/282; A61K 31/337; A61K 31/426; A61K 31/427; A61K 31/4439; A61K 31/454; A61K 31/496; A61K 31/5377; A61K 31/704; A61K 38/05; A61P 35/00; C07D 277/56; C07D 417/12; C07D 417/14

(57) ABSTRACT

Provided according to some embodiments is a compound of Formula (1), a 2-aryl, 2-heteroaryl, 2-cycloalkyl or 2-heterocycle substituted thiazole-4-carboxamido) acrylamido)acrylate compound, as a peroxiredoxin 3 (PRX3) inhibitor, or a pharmaceutically acceptable salt or prodrug thereof. Pharmaceutical compositions comprising the same and methods of use for treating cancer and inhibiting PRX3 in a subject in need thereof, are also provided.

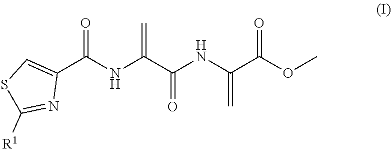

### STATEMENT OF GOVERNMENT SUPPORT

This invention was made with government support under Contract Number R01 GM072866 awarded by the National Institutes of Health. The U.S government has certain rights in the invention.

### BACKGROUND

A central hallmark of the tumorigeneses of cells is metabolic changes that cause an increase in the level of reactive oxygen species (ROS) and mitochondrial ROS (mROS). Cairns et al. (2011) Nat Rev Cancer 11, 85-95; Weinberg et al. (2009) Cell Mol Life Sci 66, 3663-3673; Weinberg et al. (2009) Ann N Y Acad Sci 1177, 66-73; Weinberg et al. (2010) Proc Natl Acad Sci USA 107, 8788-8793. In response to this neoplastic transformation, cells reorganize their antioxidant capacity to survive, proliferate and metastasize. Specifically, oncogene-induced increases in ROS levels activate the oncogenic transcription factor FOXM1, inducing the expression of FOXM1 target genes including the mitochondrial antioxidant enzymes superoxide dismutase 2 and peroxiredoxin 3 (PRX3). Park et al. (2009) EMBO J 28, 2908-2918; Nonn et al. (2003) Mol Cancer Res 1, 682-689.

PRX3 is a peroxidase responsible for metabolizing ˜90% of mitochondrial hydrogen peroxide (H₂O₂) (Cox et al. (2009) Biochem J 425, 313-325), and this specific ROS is known to regulate several important processes involved in tumor progression including proliferation, apoptosis, migration and metastasis. The GEPIA2 database of matched pairs of patient samples (tumor vs. normal) illustrates how the PRX3 transcript levels are elevated in 15/32 (46.9%) of the tumor tissues collected, including many forms of cancer with significant unmet medical need. Tang et al. (2019) Nucleic Acids Res 47, W556-W560. PRX3 protein expression and mROS levels correlate with sensitivity to the natural product and PRX3 inhibitor thiostrepton (TS) in patient-derived malignant mesothelioma cells lines. Nelson et al. (2021) Antioxidants (Basel) 10, 150.

PRX3 expression supports malignant mesothelioma (MM) and ovarian tumor (OvCa) cell growth. Cunniff et al. (2015) PloS one 10, e0127310; Myers (2016) Free Radic Biol Med 91, 81-92; Yoshikawa et al. (2016) Oncol Rep 35, 2543-2552; Wang et al. (2013) Tumour Biol 34, 2275-2281. PRX3 expression levels in OvCa and cervical cancer also correlate with poor patient outcomes. Li et al. (2018) Biosci Rep 38. The following additional features further support PRX3 as a promising molecular target for cancer therapy: (i) no cancer mutations in the PRX3 gene known to support resistance development; (ii) PRX3 KO mice are viable and reach maturity; increase in basal oxidative stress levels observed only in a variety of challenge models (Li et al. (2007) Biochem Biophys Res Commun 355, 715-721; Lee (2020) Antioxidants (Basel) 9); and (iii) partial knockdown of PRX3 via shRNA slows tumor cell proliferation and significantly reduced the expression of FOXM1 at the RNA and protein levels (Cunniff et al. (2015) PloS one 10, e0127310).

A study by Corsello et al. (Nature Cancer 2020 1(2):235-248) tested the ability of 4,518 drugs from the Drug Repurposing Hub at the Broad Institute to kill 578 cancer cells lines. Thiostrepton (TS), an insoluble, thiopeptide antibiotic, showed meaningful efficacy in 403 tumor cells lines derived from a wide array of tissues. Our team has demonstrated that TS acts by irreversibly crosslinking the two essential catalytic cysteine residues in PRX3, inactivating peroxidase activity and increasing ROS to levels incompatible with survival. Nelson et al. (2021) Antioxidants (Basel) 10, 150; Cunniff et al. (2015) PloS one 10, e0127310; Newick et al. (2012) PloS one 7, e39404. Because this irreversible crosslink occurs across the homodimer interface, the inactivated PRX3 is significantly larger in mass, and we can track the PRX3 crosslink in our cellular and animal models.

Several mechanisms have been proposed for TS cytotoxicity of cancer cells: (i) interaction with the oncogenic transcription factor FOXM1 (Hegde et al. (2011) Nat Chem 3, 725-731) (ii) inhibition of the 20/26S proteasome (Bhat et al. (2009) PloS one 4, e6593; Bird et al. (2020) ACS Chem Biol 15, 2164-2174), (iii) binding to the large subunit of ribosomes (Zhang et al. (2005) Antibiotic susceptibility of mammalian mitochondrial translation. FEBS Lett 579, 6423-6427; Harms et al. (2008) Mol Cell 30, 26-38), and (iv) covalent adduction and cross-linking of PRX3 by our team (Nelson et al. (2021) Antioxidants (Basel) 10, 150; Cunniff et al. (2015) PloS one 10, e0127310). We have shown that TS sensitivity is greatly decreased upon knockdown of PRX3 in a cell model of MM, indicating that PRX3 inhibition is key in driving TS cytotoxicity. Inhibition of PRX3 also significantly increases mitochondrial ROS which drives TS-mediated cell death. Increased ROS modulates FOXM1 expression while increased production of mitochondrial ROS has also been shown to disassemble 26S proteasome complexes (Livnat-Levanon et al. (2014) Cell Rep 7, 1371-1380; Segref et al. (2014) Cell Metab 19, 642-652), further complicating the interpretation of the mode of action of TS.

Despite the effectiveness of TS in cell and animal models of cancer, this natural product has serious limitations to its utility as a chemotherapy. First, it is highly large, highly insoluble, and does not exhibit any of the preferred drug like properties. Second, this molecule is currently produced by bacterial fermentation followed by organic extraction and purification. Although a synthetic route for synthesis has been published, it involved multiple steps, is expensive, and has low yield. Ayida et al. (2005) Bioorg Med Chem Lett 15, 2457-2460.

Improved PRX3 inhibitors that can address some of these issues are needed.

### SUMMARY

Provided herein according to some embodiments is a compound of Formula I:

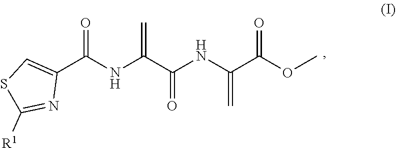

wherein R¹ is an aryl, heteroaryl, cycloalkyl or heterocycle, which aryl, heteroaryl, cycloalkyl or heterocycle is optionally substituted with one or more selected from alkyl, carboxy, carbamate, urea, amide, amino, ether, ester and halo, or a pharmaceutically acceptable salt or prodrug thereof. In some embodiments, when R¹ is pyridine or pyrazine, said pyridine or pyrazine is substituted with one or more selected from alkyl, carboxy, carbamate, urea, amide, amino, ether, ester and halo.

In some embodiments, the aryl, heteroaryl, cycloalkyl or heterocycle, is substituted with one or more selected from alkyl, carboxy, carbamate, urea, amide, and halo. In some embodiments, the aryl, heteroaryl, cycloalkyl or heterocycle, is substituted with carbamate or amide. In some embodiments, the aryl, heteroaryl, cycloalkyl or heterocycle, is substituted with an alkylcarbamate.

In some embodiments, R¹ is a group having a structure of:

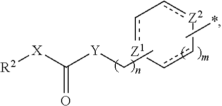

wherein:
n is 0, 1, 2 or 3;
m is 0, 1 or 2;
X is absent or is O, NR³, or CH₂;
Y is absent or is O, NR³, or CH₂;
Z¹ and Z² are each independently O, N, or C;
R² is alkyl (e.g., having from 1 to 8 carbon atoms, linear or branched), wherein said alkyl is optionally substituted (e.g., with halo, amino, ether, alkoxy, or carbamate), or heterocycle; and
R³ is H or alkyl (e.g., having from 1 to 8 carbon atoms, linear or branched),
wherein * denotes the connection of the group in the compound of Formula I.

In some embodiments, Z¹ and Z² are each independently N or C.

In some embodiments, R¹ is a group having a structure of:

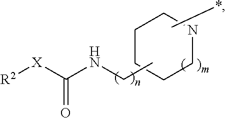

wherein:
n is 0, 1, 2 or 3;
m is 0, 1 or 2;
X is O or CH₂; and
R² is alkyl (e.g., having from 1 to 8 carbon atoms, linear or branched), wherein said alkyl is optionally substituted (e.g., with halo, amino, ether, alkoxy, or carbamate), or heterocycle,
wherein * denotes the connection of the group in the compound of Formula I.

In some embodiments, R¹ is a group having a structure of:

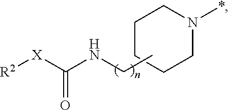

wherein:
n is 0, 1, 2 or 3;
X is O or CH₂; and
R² is alkyl (e.g., having from 1 to 8 carbon atoms, linear or branched), wherein said alkyl is optionally substituted (e.g., with halo, amino, ether, alkoxy, or carbamate), or heterocycle,
wherein * denotes the connection of the group in the compound of Formula I, or a pharmaceutically acceptable salt or prodrug thereof.

In some embodiments, R¹ is a group having a structure of:

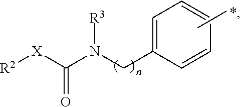

wherein:
n is 0, 1, 2 or 3;
X is O or CH₂;
R² is alkyl (e.g., having from 1 to 8 carbon atoms, linear or branched), wherein said alkyl is optionally substituted (e.g., with halo, amino, ether, alkoxy, or carbamate), or heterocycle; and
R³ is H or alkyl (e.g., having from 1 to 8 carbon atoms, linear or branched),
wherein * denotes the connection of the group in the compound of Formula I.

Also provided is a pharmaceutical composition comprising a compound or pharmaceutically acceptable salt or prodrug as taught herein. In some embodiments, the composition is formulated for oral or parenteral (e.g. intravenous, intrapleural, intraperitoneal or intraovarian) administration. In some embodiments, the composition is formulated for oral administration and is in the form of a capsule, cachet, lozenge, or tablet. In some embodiments, the formulation is provided in unit dosage form of from 1 mg to 10 grams of the compound, pharmaceutically acceptable salt or prodrug.

Further provided is a method treating cancer in a subject in need thereof, comprising administering to the subject a therapeutically effective amount of a compound of Formula I, or a pharmaceutically acceptable salt or prodrug thereof. Also provided is a compound of Formula I or a pharmaceutically acceptable salt or prodrug thereof for use in treating cancer in a subject in need thereof, or for preparing a medicament for use in treating cancer.

In some embodiments, the cancer has PRX3 expression.

In some embodiments, the subject is a human subject. In some embodiments, the subject is a non-human animal subject (e.g. non-human mammalian subject).

In some embodiments, the administering is carried out by administering a pharmaceutical composition comprising said compound or pharmaceutically acceptable salt or prodrug.

In some embodiments, the administering further comprises administering bortezomib, carboplatin, paclitaxel, an immunotherapy agent, or a combination thereof. In some embodiments, the administering further comprises administering doxorubicin.

Further provided is a method of inhibiting PRX3 in a subject in need thereof, comprising administering to said subject a therapeutically effective amount of a compound of Formula I, or a pharmaceutically acceptable salt or prodrug thereof.

### DETAILED DESCRIPTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

The disclosures of all patent references cited herein are hereby incorporated by reference to the extent they are consistent with the disclosure set forth herein. As used herein in the description of the invention and the appended claims, the singular forms “a,” “an” and “the” are intended to include the plural forms as well, unless the context clearly indicates otherwise.

### I. Definitions

As used herein in the accompanying chemical structures, “H” refers to a hydrogen atom. “C” refers to a carbon atom. “N” refers to a nitrogen atom. “S” refers to a sulfur atom. “O” refers to an oxygen atom.

Unless indicated otherwise, nomenclature used to describe chemical groups or moieties as used herein follow the convention where, reading the name from left to right, the point of attachment to the rest of the molecule is at the right hand side of the name. For example, the group “alkylamino” is attached to the rest of the molecule at the amino end, whereas the group “aminoalkyl” is attached to the rest of the molecule at the alkyl end.

“Alkyl,” as used herein, refers to a saturated straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. “Lower alkyl” as used herein, is a subset of alkyl and refers to a straight or branched chain hydrocarbon group containing from 1 to 4 carbon atoms. Representative examples of lower alkyl include, but are not limited to, methyl, ethyl, n-propyl, iso-propyl, n-butyl, iso-butyl, tert-butyl, and the like. The alkyl groups may be optionally substituted with one or more suitable substituents, such as halo, hydroxy, carboxy, amine, etc.

“Cycloalkyl,” as used herein, refers to a saturated cyclic hydrocarbon containing from 1 to 10 carbon atoms. Representative examples of cycloalkyl include, but are not limited to, cyclopropyl, cyclobutyl, cyclopentyl, cyclohexyl, cycloheptyl, cyclooctyl, and the like. The cycloalkyl groups may be optionally substituted with one or more suitable substituents, such as halo, hydroxy, carboxy, amine, etc.

“Aryl,” as used herein, refers to a monocyclic carbocyclic ring system or a bicyclic carbocyclic fused or directly adjoining ring system having one or more aromatic rings. Examples include, but are not limited to, phenyl, indanyl, indenyl, tetrahydronaphthyl, biphenyl, napthyl, azulenyl, etc. The aryl may be optionally substituted with one or more suitable substituents, such as alkyl, halo, hydroxy, carboxy, amine, etc.

“Heteroaryl,” as used herein, refers to a monovalent aromatic group having a single ring or two fused or directly adjoining rings and containing in at least one of the rings at least one heteroatom (typically 1 to 3) independently selected from nitrogen, oxygen and sulfur. Examples include, but are not limited to, pyrrole, imidazole, thiazole, oxazole, furan, thiophene, triazole, pyrazole, isoxazole, isothiazole, pyridine, pyrazine, pyridazine, pyrimidine, triazine, benzothiophene, benzofuran, indole, benzimidazole, benzothiazole, quinoline, isoquinoline, quinazoline, quinoxaline, phenyl-pyrrole, phenyl-thiophene, etc. The heteroaryl may be optionally substituted with one or more suitable substituents, such as alkyl, halo, hydroxy, carboxy, amine, etc.

“Heterocycle” as used herein refers to a saturated or partially unsaturated cyclic hydrocarbon with at least one heteroatom (typically 1 to 3) independently selected from nitrogen, oxygen and sulfur. The heterocycle may be a monocyclic heterocycle, a bicyclic heterocycle, or a tricyclic heterocycle. The heterocycle may be optionally substituted with one or more suitable substituents, such as alkyl, halo, hydroxy, carboxy, amine, etc.

“Monocyclic heterocycle” means a 3-, 4-, 5-, 6-, 7-, or 8-membered ring containing at least one heteroatom, and which is not aromatic. Representative examples of monocyclic heterocycle include, but are not limited to, azetidinyl, azepanyl, aziridinyl, diazepanyl, 1,3-dioxanyl, 1,3-dioxolanyl, dihydropyranyl (including 3,4-dihydro-2H-pyran-6-yl), 1,3-dithiolanyl, 1,3-dithianyl, imidazolidinyl, isothiazolidinyl, isoxazolidinyl, morpholinyl, oxadiazolidinyl, oxazolidinyl, piperazinyl, piperidinyl, pyranyl, pyrazolidinyl, pyrrolidinyl, tetrahydrofuranyl, tetrahydropyranyl (including tetrahydro-2H-pyran-4-yl), tetrahydrothienyl, thiadiazolidinyl, thiazolidinyl, thiomorpholinyl, 1,1-dioxidothiomor-pholinyl (thiomorpholine sulfone), thiopyranyl, and trithianyl.

“Bicyclic heterocycle” means a monocyclic heterocycle fused to an aryl group, or a monocyclic heterocycle fused to a monocyclic cycloalkyl or cycloalkenyl, or a monocyclic heterocycle fused to a monocyclic heterocycle. Representative examples of bicyclic heterocycles include, but are not limited to, 3,4-dihydro-2H-pyranyl, 1,3-benzodioxolyl, 1,3-benzodithiolyl, 2,3-dihydro-1,4-benzodioxinyl, 2,3-dihydro-1-benzofuranyl, 2,3-dihydro-1-benzothienyl, 2,3-dihydro-1H-indolyl, 3,4-dihydroquinolin-2(1H)-one and 1,2,3,4-tetrahydroquinolinyl.

“Tricyclic heterocycle” means a bicyclic heterocycle fused to an aryl group, or a bicyclic heterocycle fused to a monocyclic cycloalkyl or cycloalkenyl, or a bicyclic heterocycle fused to a monocyclic heterocycle. Representative examples of tricyclic heterocycles include, but are not limited to, 2,3,4,4a,9,9a-hexahydro-1H-carbazolyl, 5a,6,7,8,9,9a-hexahydrodibenzo[b,d]furanyl, and 5a,6,7,8,9,9a-hexahydrodibenzo[b,d]thienyl.

The terms “halo” and “halogen” refer to fluoro (—F), choro (—Cl), bromo (—Br), or iodo (—I).

“Haloalkyl” refers to one or more halo groups appended to the parent molecular moiety through an alkyl group. Examples include, but are not limited to, chloromethyl, fluoromethyl, trifluoromethyl, etc.

“Carboxy” refers to the group —COOH.

“Alkoxy” refers to an alkyl or cycloalkyl group, as herein defined, attached to the principal carbon chain through an oxygen atom. Representative examples of “alkoxy” include, but are not limited to, methoxy, ethoxy, propoxy, 2-propoxy, butoxy, tert-butoxy, and hexyloxy.

“Hydroxy” or “hydroxyl” refers to an —OH group.

An “amine” or “amino” refers to a group —NH₂, wherein none, one or two of the hydrogens may be replaced by an alkyl, cycloalkyl, or aryl as defined herein.

An “amide” or “amido” refers to a group having a carbonyl bonded to a nitrogen atom, such as —C(O)NH₂, wherein none, one or two of the hydrogens may be replaced by an alkyl, cycloalkyl, heterocycle, or aryl as defined herein.

An “ether” refers to a group in which there is an ether, R—O—R′, wherein R and R′ are each independently an alkyl, cycloalkyl, or aryl as defined herein.

An “ester” refers to a group in which there is an ester, R—C(O)—O—R′, wherein R and R′ are each independently an alkyl, cycloalkyl, or aryl as defined herein.

A “carbamate” refers to a group in which there is a carbamate, R—O—C(O)NR′R″, wherein R, R′ and R″ are each independently an alkyl, cycloalkyl, or aryl as defined herein.

A “urea” refers to a group in which there is a urea, R—NH—C(O)—NH—R′, wherein R and R′ are each independently an alkyl, cycloalkyl, or aryl as defined herein.

As understood in the art, the term “optionally substituted” indicates that the specified group is either unsubstituted, or substituted by one or more suitable substituents. A “substituent” that is “substituted” is a group which takes the place of one or more hydrogen atoms on the parent organic molecule.

Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (b) salts formed from elemental anions such as chlorine, bromine, and iodine.

### II. Active Compounds

Active compounds useful as PRX3 inhibitors in accordance with the present invention are provided below. Unless otherwise stated, structures depicted herein are also meant to include all enantiomeric, diastereomeric, and geometric (or conformational) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Tautomeric forms include keto-enol tautomers of a compound. In addition, unless otherwise stated, all rotamer forms of the compounds of the invention are within the scope of the invention.

Unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a 13C- or 14C-enriched carbon are within the scope of this invention. Such compounds are useful, for example, as analytical tools or probes in biological assays.

Provided herein according to some embodiments as an active compound is a compound of Formula I:

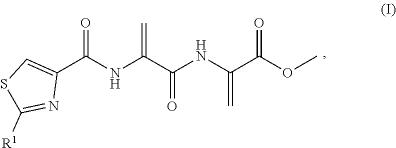

wherein R¹ is an aryl, heteroaryl, cycloalkyl or heterocycle, which aryl, heteroaryl, cycloalkyl or heterocycle is optionally substituted with one or more selected from alkyl, carboxy, carbamate, urea, amide, amino, ether, ester and halo, or a pharmaceutically acceptable salt or prodrug thereof. In some embodiments, when R¹ is pyridine or pyrazine, said pyridine or pyrazine is substituted with one or more selected from alkyl, carboxy, carbamate, urea, amide, amino, ether, ester and halo.

In some embodiments, the aryl, heteroaryl, cycloalkyl or heterocycle, is substituted with one or more selected from alkyl, carboxy, carbamate, urea, amide, and halo. In some embodiments, the aryl, heteroaryl, cycloalkyl or heterocycle, is substituted with carbamate or amide. In some embodiments, the aryl, heteroaryl, cycloalkyl or heterocycle, is substituted with an alkylcarbamate.

In some embodiments, R¹ is a group having a structure of:

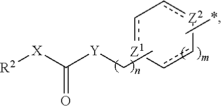

wherein:
n is 0, 1, 2 or 3;
m is 0, 1 or 2;
X is absent or is O, NR³, or CH₂;
Y is absent or is O, NR³, or CH₂;
Z¹ and Z² are each independently O, N, or C;
R² is alkyl (e.g., having from 1 to 8 carbon atoms, linear or branched), wherein said alkyl is optionally substituted (e.g., with halo, amino, ether, alkoxy, or carbamate), or heterocycle; and
R³ is H or alkyl (e.g., having from 1 to 8 carbon atoms, linear or branched),
wherein * denotes the connection of the group in the compound of Formula I (i.e., the connection of the group at the 2-position of the thiazole ring).

In some embodiments, Z¹ and Z² are each independently N or C.

Particular examples of active compounds include, but are not limited to, those selected from the group consisting of:

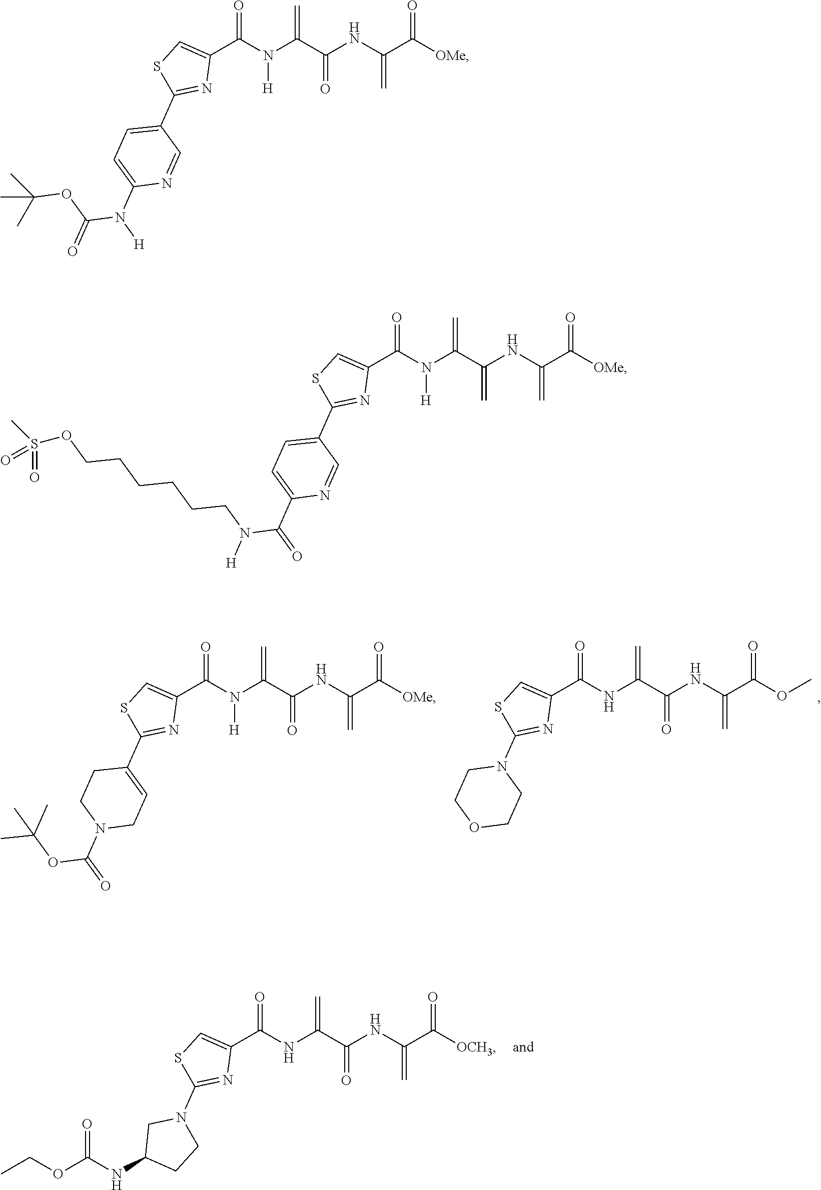 [SMILES: C=C(NC(=O)c1csc(-c2ccc(NC(=O)OC(C)(C)C)nc2)n1)C(=O)NC(=C)C(=O)OC ; C=C(NC(=C)C(=O)OC)C(=C)NC(=O)c1csc(-c2ccc(C(=O)NCCCCCCOS(C)(=O)=O)nc2)n1 ; C=C(NC(=O)c1csc(C2=CCN(C(=O)OC(C)(C)C)CC2)n1)C(=O)NC(=C)C(=O)OC ; C=C(NC(=O)c1csc(N2CCOCC2)n1)C(=O)NC(=C)C(=O)OC ; C=C(NC(=O)c1csc(N2CC[C@@H](NC(=O)OCC)C2)n1)C(=O)NC(=C)C(=O)OC]

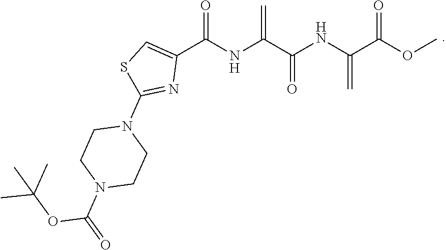 [SMILES: C=C(NC(=O)c1csc(N2CCN(C(=O)OC(C)(C)C)CC2)n1)C(=O)NC(=C)C(=O)OC]

In some embodiments, R¹ is a group having a structure of:

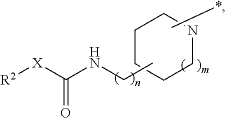

wherein:
n is 0, 1, 2 or 3;
m is 0, 1 or 2;
X is O or CH₂; and
R² is alkyl (e.g., having from 1 to 8 carbon atoms, linear or branched), wherein said alkyl is optionally substituted (e.g., with halo, amino, ether, alkoxy, or carbamate), or heterocycle; and
wherein * denotes the connection of the group in the compound of Formula I (i.e., the connection of the group at the 2-position of the thiazole ring).

In some embodiments, R¹ is a group having a structure of:

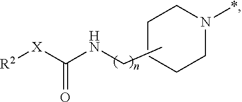

wherein:
n is 0, 1, 2 or 3;
X is O or CH₂; and
R² is alkyl (e.g., having from 1 to 8 carbon atoms, linear or branched), wherein said alkyl is optionally substituted (e.g., with halo, amino, ether, alkoxy, or carbamate), or heterocycle; and
wherein * denotes the connection of the group in the compound of Formula I (i.e., the connection of the group at the 2-position of the thiazole ring).

Particular examples of active compounds include, but are not limited to, those selected from the group consisting of:

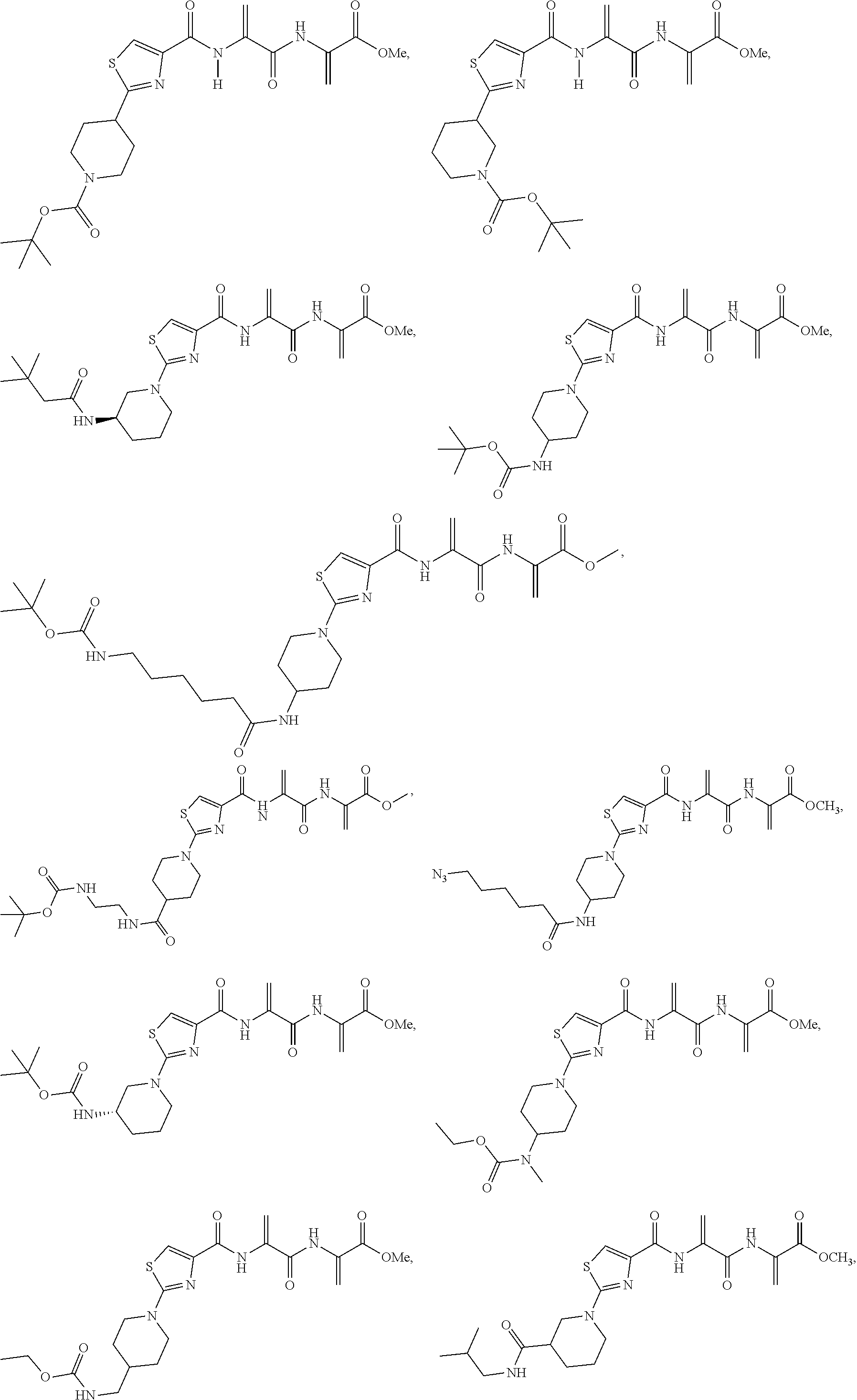

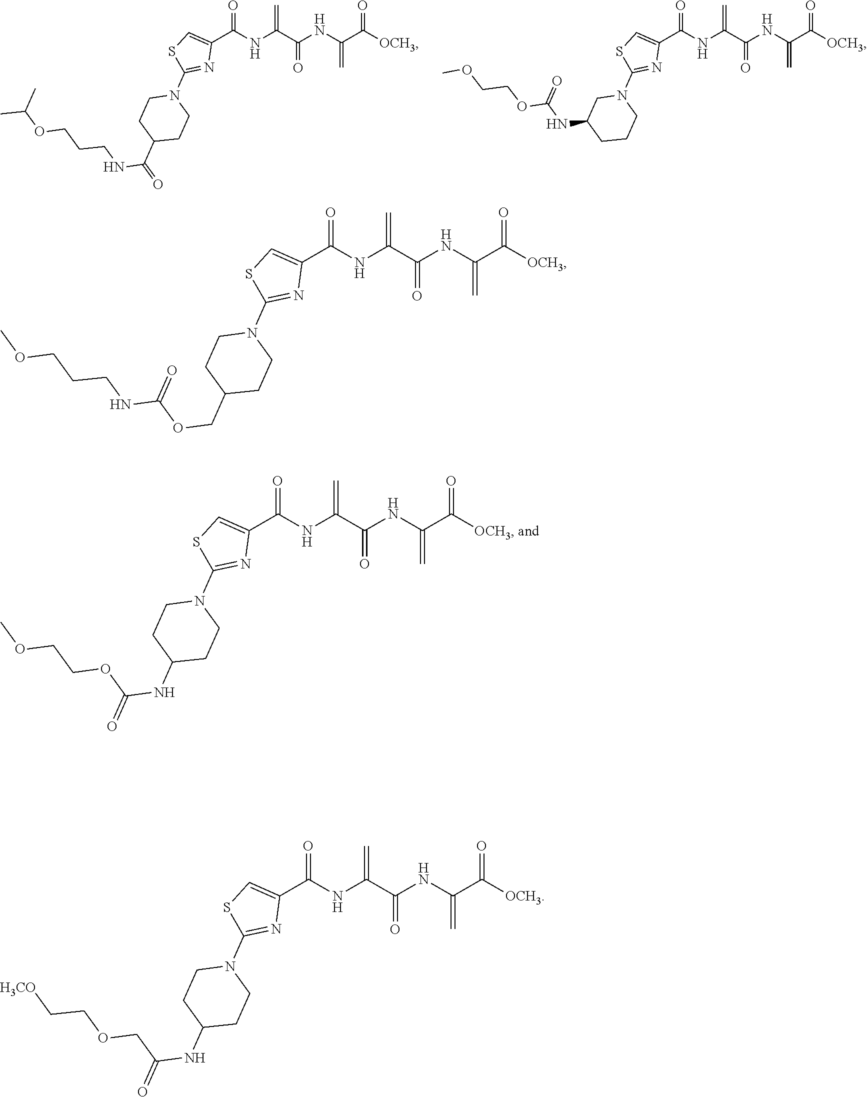

In some embodiments, R¹ is a group having a structure of:

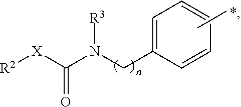

wherein:
n is 0, 1, 2 or 3;
X is O or CH₂;
R² is alkyl (e.g., having from 1 to 8 carbon atoms, linear or branched), wherein said alkyl is optionally substituted (e.g., with halo, amino, ether, alkoxy, or carbamate), or heterocycle; and
R³ is H or alkyl (e.g., having from 1 to 8 carbon atoms, linear or branched),
wherein * denotes the connection of the group in the compound of Formula I (i.e., the connection of the group at the 2-position of the thiazole ring).

Particular examples of active compounds include, but are not limited to, those selected from the group consisting of:

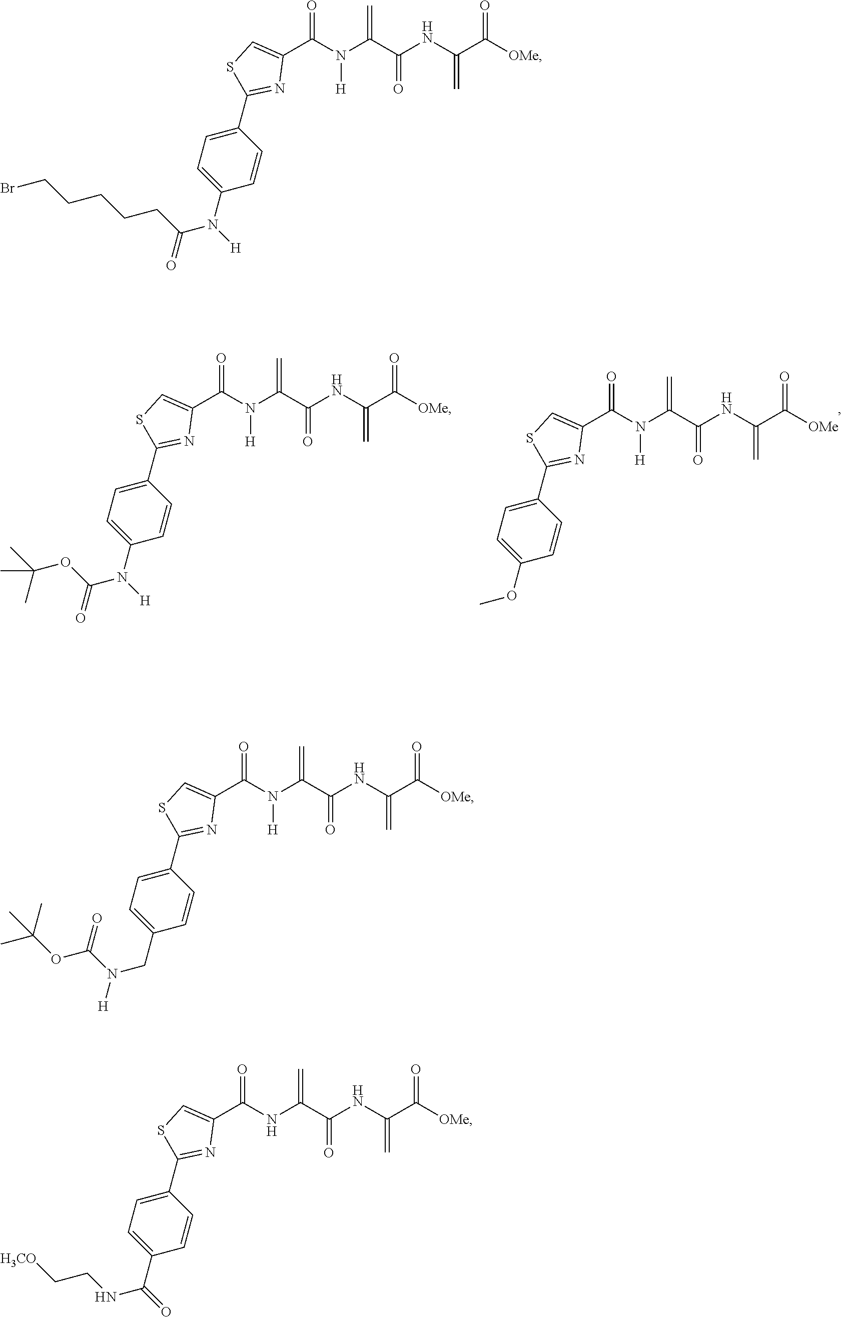 [SMILES: C=C(NC(=O)c1csc(-c2ccc(NC(=O)CCCCCBr)cc2)n1)C(=O)NC(=C)C(=O)OC ; C=C(NC(=O)c1csc(-c2ccc(NC(=O)OC(C)(C)C)cc2)n1)C(=O)NC(=C)C(=O)OC ; C=C(NC(=O)c1csc(-c2ccc(OC)cc2)n1)C(=O)NC(=C)C(=O)OC ; C=C(NC(=O)c1csc(-c2ccc(CNC(=O)OC(C)(C)C)cc2)n1)C(=O)NC(=C)C(=O)OC ; C=C(NC(=O)c1csc(-c2ccc(C(=O)NCCOC)cc2)n1)C(=O)NC(=C)C(=O)OC]

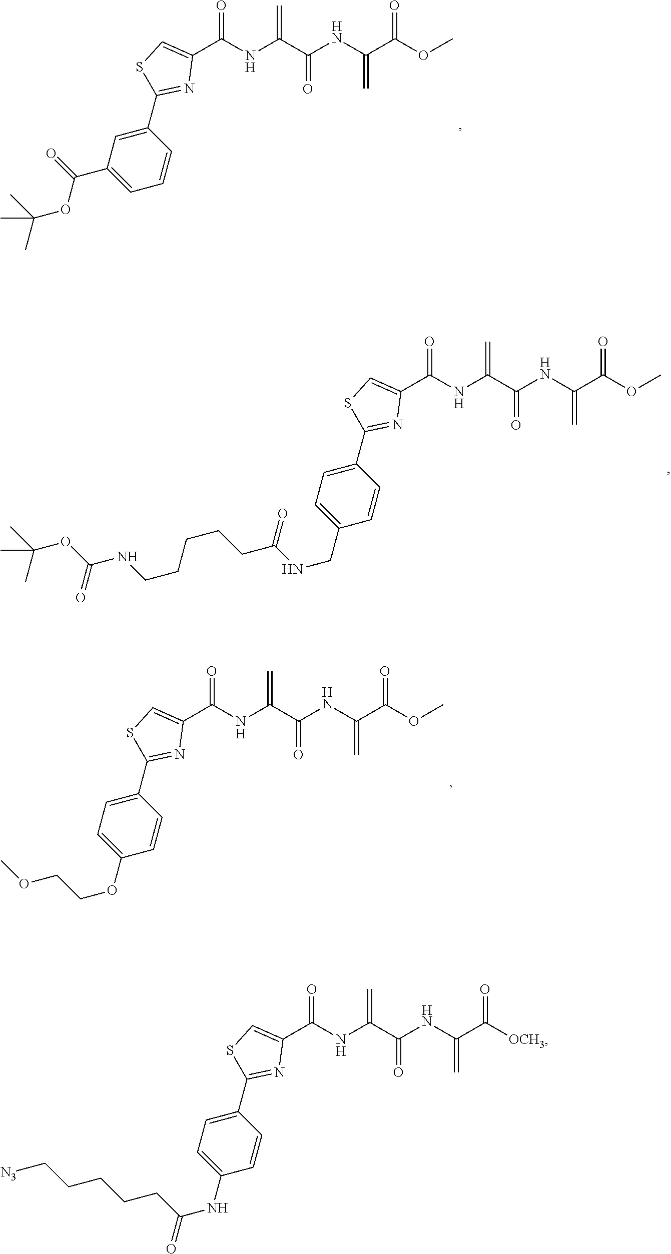

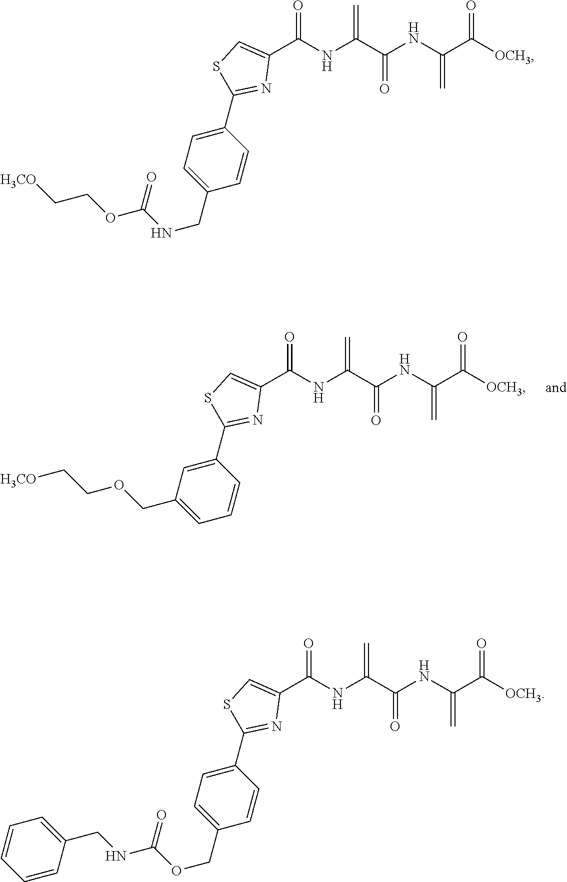 [SMILES: C=C(NC(=O)c1csc(-c2ccc(CNC(=O)OCCOC)cc2)n1)C(=O)NC(=C)C(=O)OC ; C=C(NC(=O)c1csc(-c2cccc(COCCOC)c2)n1)C(=O)NC(=C)C(=O)OC ; C=C(NC(=O)c1csc(-c2ccc(COC(=O)NCc3ccccc3)cc2)n1)C(=O)NC(=C)C(=O)OC]

Particular examples of active compounds include, but are not limited to, those selected from the group consisting of:

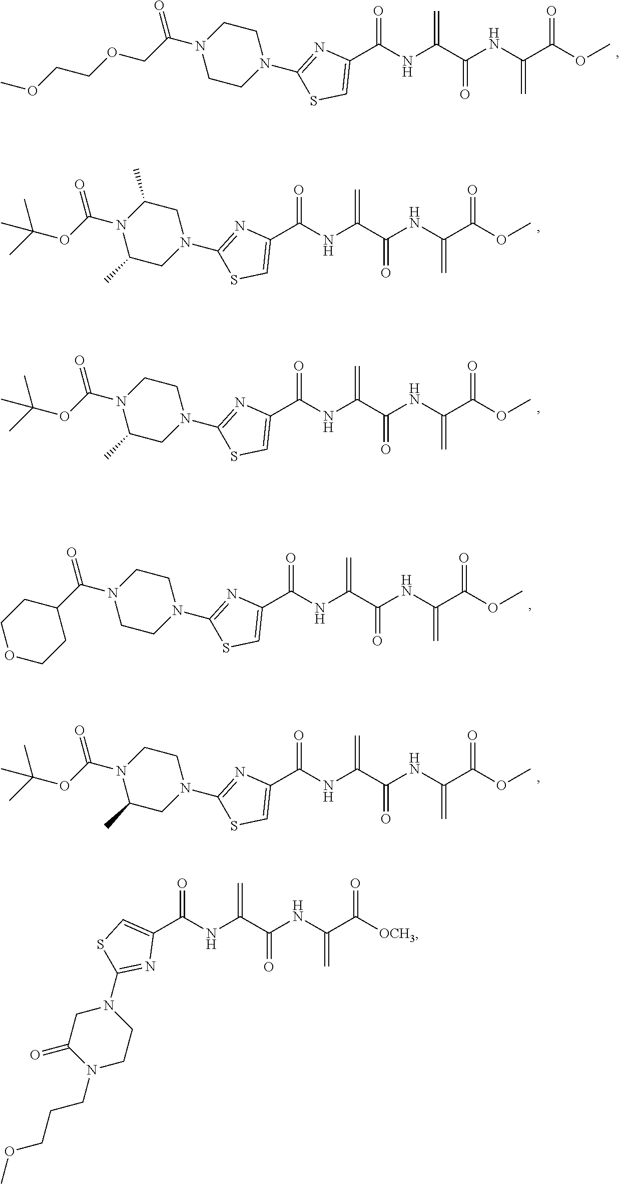

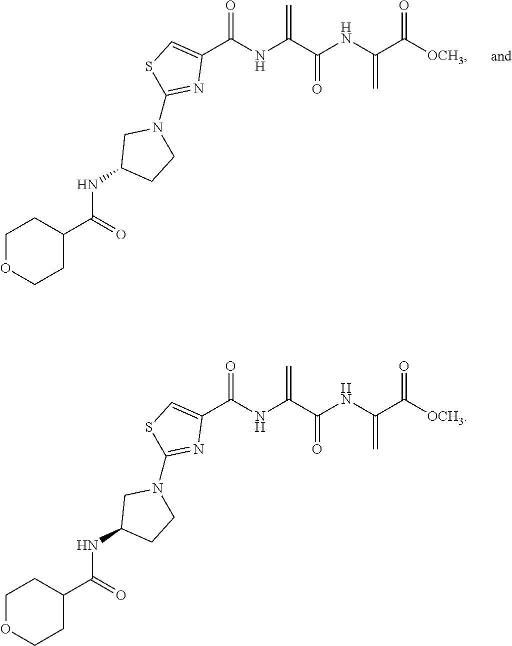 [SMILES: C=C(NC(=O)c1csc(N2CC[C@H](NC(=O)C3CCOCC3)C2)n1)C(=O)NC(=C)C(=O)OC ; C=C(NC(=O)c1csc(N2CC[C@@H](NC(=O)C3CCOCC3)C2)n1)C(=O)NC(=C)C(=O)OC]

Particular examples of active compounds include, but are not limited to, those selected from the group consisting of:

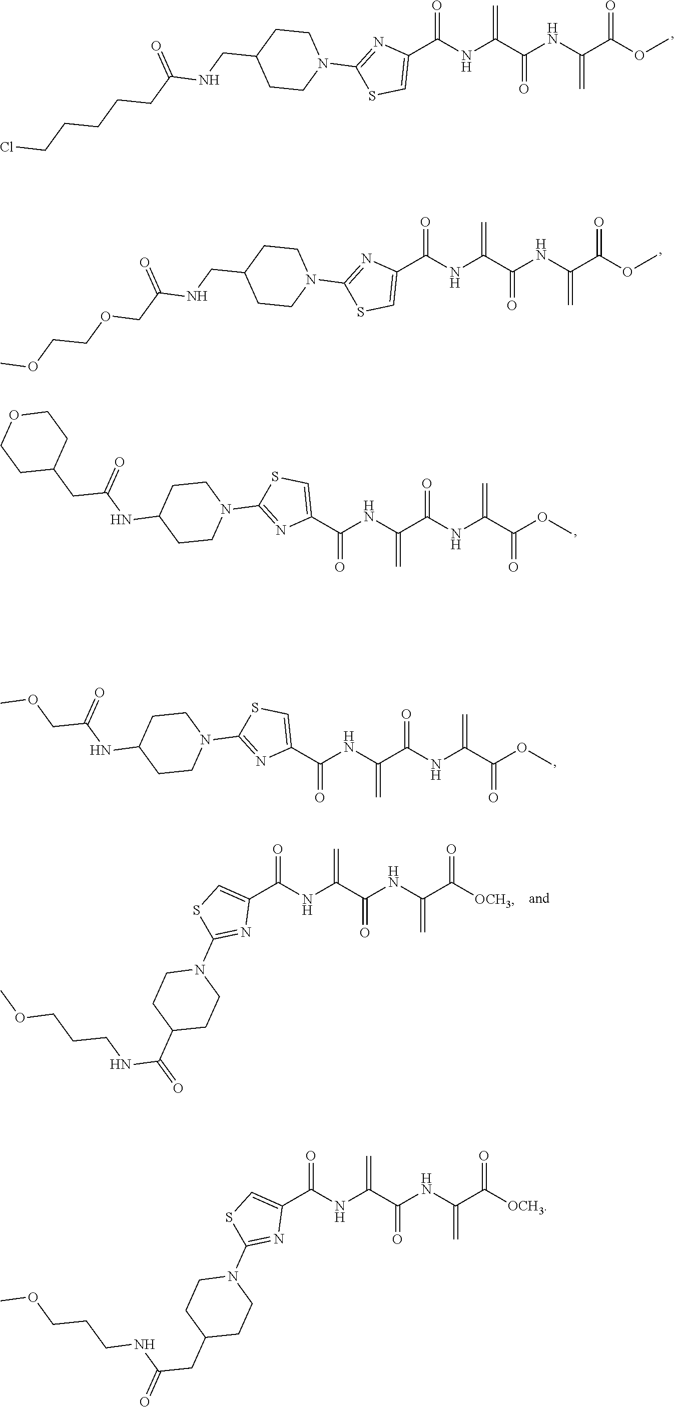

Particular examples of active compounds include, but are not limited to, those selected from the group consisting of:

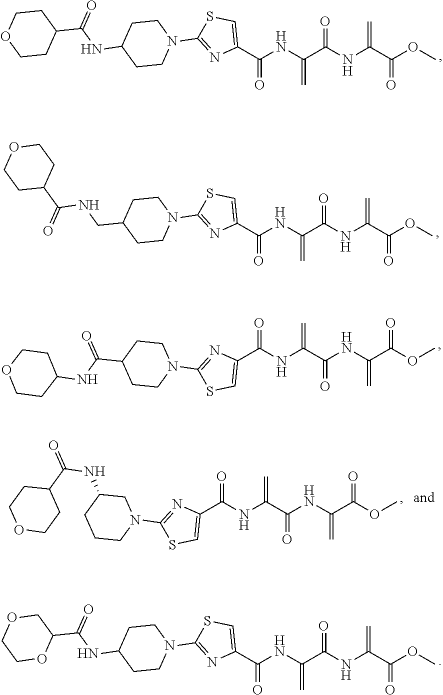 [SMILES: C=C(NC(=O)c1csc(N2CCC(NC(=O)C3CCOCC3)CC2)n1)C(=O)NC(=C)C(=O)OC ; C=C(NC(=O)c1csc(N2CCC(CNC(=O)C3CCOCC3)CC2)n1)C(=O)NC(=C)C(=O)OC ; C=C(NC(=O)c1csc(N2CCC(C(=O)NC3CCOCC3)CC2)n1)C(=O)NC(=C)C(=O)OC ; C=C(NC(=O)c1csc(N2CCC[C@H](NC(=O)C3CCOCC3)C2)n1)C(=O)NC(=C)C(=O)OC ; C=C(NC(=O)c1csc(N2CCC(NC(=O)C3COCCO3)CC2)n1)C(=O)NC(=C)C(=O)OC]

Particular examples of active compounds include, but are not limited to, those selected from the group consisting of:

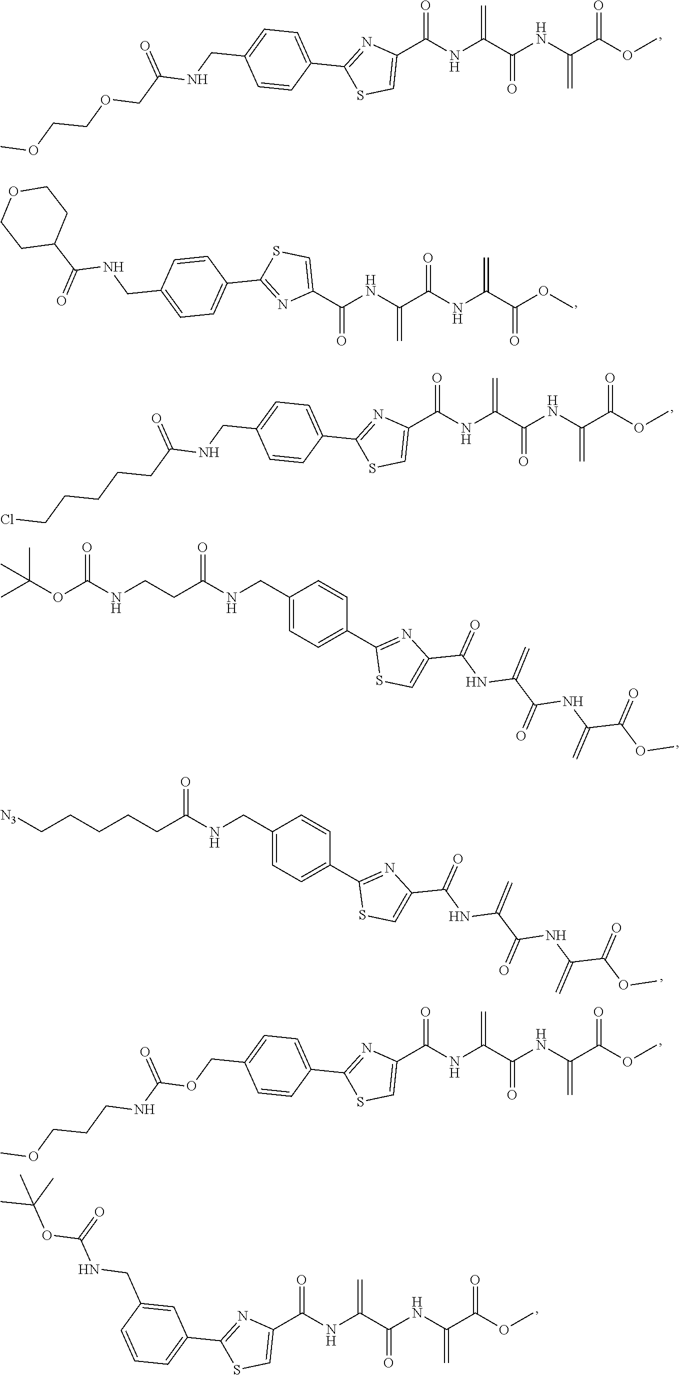

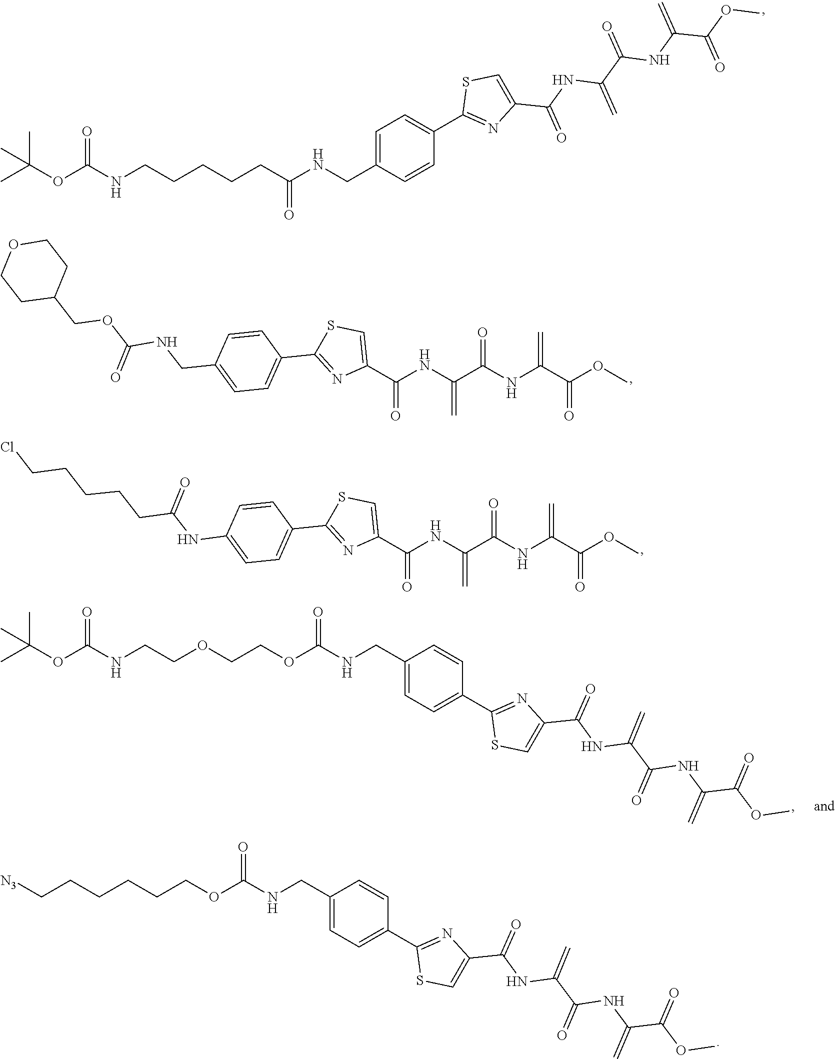

In some embodiments, an active compound can form a covalent adduct with PRX3 in a biochemical PRX3 inhibition assay, which may support its PRX3 inhibition activity.

In some embodiments, an active compound can have an EC₅₀ in a cellular activity assay (such as the ability to kill cancer cells such as SKOV3 ovarian cancer cells) in the micromolar range, such as from 0.05, 0.1, 0.25, or 0.5 micromolar, to 10, 15, or 20 micromolar.

In some embodiments, an active compound can have good solubility, e.g., solubility in aqueous solution (e.g., saline such as phosphate buffered saline, water, etc.) of at least 0.1 millimolar, such as from 0.1 to 1 millimolar, or solubility in an aqueous solution of at least 1 millimolar.

In some embodiments, an active compound can have good aqueous stability. For example, in some embodiments, an active compound may have no decrease in purity after 24 hours in an aqueous solution.

In some embodiments, an active compound does not have appreciable antimicrobial activity, e.g., at greater than 20 micromolar concentrations.

In some embodiments, an active compound does not inhibit the proteasome and/or does not inhibit FOXM1 DNA binding. These may indicate that the compound has greater specificity for PRX3 than TS.

### III. Methods of Use

The terms “treat”, “treatment” and “treating” as used herein refer to any type of treatment that imparts a benefit to a subject afflicted with a disease or disorder, delay in the progression of the disease or disorder, or symptoms thereof, etc. In some embodiments, the treatment is for a cancer (e.g., a cancer having elevated reactive oxygen species).

In some embodiments, the subject treated is a human subject. In some embodiments, the subject is a non-human animal (e.g., non-human mammalian subject). A non-human animal may include, but is not limited to, non-human primates, dogs, cats, horses, cattle, goats, pigs, sheep, guinea pigs, mice, rats and rabbits, as well as any other domestic, commercially or clinically valuable animal, including but not limited to animal models and livestock animals. In some embodiments, the subject is a subject in need of a treatment such as a treatment of the present invention.

Cancers that may be treated with the active compounds according to some embodiments may include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bile duct cancer; bladder cancer; bone cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cardiac tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ductal carcinoma in situ; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; multiple myeloma; heavy chain disease (e.g., alpha chain disease, gamma chain disease, mu chain disease); hemangioblastoma; histiocytosis; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); melanoma; midline tract carcinoma; multiple endocrine neoplasia syndrome; muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); nasopharynx cancer; neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); parathyroid cancer; papillary adenocarcinoma; penile cancer (e.g., Paget's disease of the penis and scrotum); pharyngeal cancer; pinealoma; pituitary cancer; pleuropulmonary blastoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; retinoblastoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; stomach cancer; small intestine cancer; sweat gland carcinoma; synovioma; testicular cancer (e.g., seminoma, testicular embryonal carcinoma); thymic cancer; thyroid cancer (e.g., papillary carcinoma of the thyroid, papillary thyroid carcinoma (PTC), medullary thyroid cancer); urethral cancer; uterine cancer; vaginal cancer; and vulvar cancer (e.g., Paget's disease of the vulva). See also US 2019/0153098 to Goldberg et al.

In some embodiments, the cancer is a blood cancer such as leukemia, liver cancer, lung cancer, lymphoma, melanoma, prostate cancer, head and neck cancer, bladder cancer, brain cancer, breast cancer, or cervical cancer. In some embodiments, the cancer is prostate cancer. In some embodiments, the cancer is head and neck cancer. In some embodiments, the cancer is ovarian cancer. In some embodiments, the cancer is cervical cancer. In some embodiments, the cancer is malignant mesothelioma.

In some embodiments, the cancer has PRX3 expression. For example, the cancer may be a cancer type generally known to express PRX3 and/or the cancer has been determined (e.g., by testing a biopsy) to have PRX3 expression.

The cancer may be metastatic, in which cancerous cells from a primary or original tumor migrate to another organ or tissue and may be identified as the tissue type of the primary or original tumor and not of that of the organ or tissue in which the secondary (metastatic) tumor is located. As a non-limiting example, a prostate cancer that has migrated to bone is said to be metastasized prostate cancer and includes cancerous prostate cancer cells growing in bone tissue.

### IV. Pharmaceutical Formulations

The active compounds disclosed herein can, as noted above, be prepared in the form of their pharmaceutically acceptable salts. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine, and (c) salts derived from bases, such as ammonium salts, alkali metal salts such as those of sodium and potassium, alkaline earth metal salts such as those of calcium and magnesium, and salts with organic bases such as dicyclohexylamine and N-methyl-D-glucamine.

Active compounds of the present invention may be prepared as pharmaceutically acceptable prodrugs. Such prodrugs are those which are, within the scope of sound medical judgment, suitable for use in contact with the tissues of humans and lower animals without undue toxicity, irritation, allergic response and the like, commensurate with a reasonable risk/benefit ratio, and effective for their intended use, as well as the zwitterionic forms, where possible, of the compounds of the invention. The term “prodrug” refers to compounds that are rapidly transformed in vivo to yield the parent compound of the above formulae, for example, by hydrolysis in blood. A thorough discussion is provided in T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, 1987, both of which are incorporated by reference herein. See also U.S. Pat. No. 6,680,299. Examples include a prodrug that is metabolized in vivo by a subject to an active drug having an activity of active compounds as described herein, wherein the prodrug is an ester of an alcohol or carboxylic acid group, if such a group is present in the compound; an acetal or ketal of an alcohol group, if such a group is present in the compound; an N-Mannich base or an imine of an amine group, if such a group is present in the compound; or a Schiff base, oxime, acetal, enol ester, oxazolidine, or thiazolidine of a carbonyl group, if such a group is present in the compound, such as described in U.S. Pat. Nos. 6,680,324 and 6,680,322.

The active compounds described above may be formulated for administration in a pharmaceutical carrier in accordance with known techniques. See, e.g., Remington, The Science and Practice of Pharmacy (9th Ed. 1995). In the manufacture of a pharmaceutical formulation according to the invention, the active compound (including the physiologically acceptable salts thereof) is typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5% to 95% or 99% by weight of the active compound. One or more active compounds may be incorporated in the formulations of the invention, which may be prepared by any of the well known techniques of pharmacy comprising admixing the components, optionally including one or more accessory ingredients.

The formulations of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces) and transdermal administration, although the most suitable route in any given case will depend on the nature, severity and location of the condition being treated and on the nature of the particular active compound which is being used.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising the active compound in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the active compound(s), which preparations are preferably isotonic with the blood of the intended recipient. These preparations may contain antioxidants, buffers, bacteriostats and solutes which render the formulation isotonic with the blood of the intended recipient. Aqueous and non-aqueous sterile suspensions may include suspending agents and thickening agents. The formulations may be presented in unit/dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising an active compound(s), or a salt thereof, in a unit dosage form in a sealed container. The compound or salt is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the compound or salt. When the compound or salt is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

Formulations suitable for rectal administration are preferably presented as unit dose suppositories. These may be prepared by admixing the active compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. Suitable formulations comprise citrate or bis/tris buffer (pH 6) or ethanol/water and contain from 0.1 to 0.2M active ingredient.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same may be incorporated into lipid vesicles. In such an instance, due to the water solubility of the compound or salt, the compound or salt will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer which forms the structure of the liposome. In either instance, the liposomes which are produced may be reduced in size, as through the use of standard sonication and homogenization techniques. The liposomal formulations containing the compounds disclosed herein or salts thereof may be lyophilized to produce a lyophilizate which may be reconstituted with a pharmaceutically acceptable carrier, such as water, to regenerate a liposomal suspension.

Other pharmaceutical compositions may be prepared from the compounds disclosed herein, or salts thereof, such as aqueous base emulsions. In such an instance, the composition will contain a sufficient amount of pharmaceutically acceptable emulsifying agent to emulsify the desired amount of the compound or salt thereof. Particularly useful emulsifying agents include phosphatidyl cholines, and lecithin.

In addition to active compound(s), the pharmaceutical compositions may contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the compositions may contain microbial preservatives. Useful microbial preservatives include methylparaben, propylparaben, and benzyl alcohol. The microbial preservative is typically employed when the formulation is placed in a vial designed for multidose use. If desired, the pharmaceutical compositions of the present invention may be lyophilized using techniques well known in the art.

### V. Dosage and Routes of Administration

As noted above, the present invention provides pharmaceutical formulations comprising the active compounds (including the pharmaceutically acceptable salts thereof), in pharmaceutically acceptable carriers for oral, rectal, topical, buccal, parenteral, intrapleural, intraovarian, intramuscular, intradermal, intravascular, and/or transdermal administration. Parenteral administration may be, for example, intravascular (intravenous or intraarterial), intrapleural, intraperitoneal or intraovarian administration by injection, infusion or implantation.

The therapeutically effective dosage of any specific compound, the use of which is in the scope of present invention, will vary somewhat from compound to compound, and patient to patient, and will depend upon the condition of the patient and the route of delivery. As a general proposition, a dosage from about 0.1 to about 50 mg/kg is expected to have therapeutic efficacy, with all weights being calculated based upon the weight of the active compound, including the cases where a salt is employed. Toxicity concerns at the higher level may restrict intravenous dosages to a lower level such as up to about 10 mg/kg, with all weights being calculated based upon the weight of the active base, including the cases where a salt is employed. A dosage from about 10 mg/kg to about 50 mg/kg may be employed for oral administration. Typically, a dosage from about 0.5 mg/kg to 5 mg/kg may be employed for intramuscular injection.

Depending upon the condition being treated, the compounds described herein may be administered alone or concurrently with one or more additional active agent useful for treating the disease or condition with which the patient is afflicted. Examples of additional active agents include, but are not limited to, those set forth in paragraphs 0065 through 0387 of W. Hunter, D. Gravett, et al., US Patent Application Publication No. 20050181977 (Published Aug. 18, 2005) (assigned to Angiotech International AG) the disclosure of which is incorporated by reference herein in its entirety.

The present invention is explained in greater detail in the following non-limiting Examples.

### EXAMPLES

We have determined the minimal fragment of TS that covalently modifies, crosslinks, and inactivates PRX3. Moreover, compounds based on this fragment inhibit PRX3, can be specific for PRX3 over other human PRX isoforms, kill multiple cancer cell types in culture, and are effective in a mouse model of malignant mesothelioma.

### Abbreviations

mmole: millimoles
g: grams
mL: milliliters
HOBt·H2O: 1-hydroxybenzotriazole hydrate
EDC·HCl: 1-(3-dimethylaminopropyl)-3-ethylcarbodiimide hydrochloride
HCl: hydrochloric acid
DCM: dichloromethane or methylene chloride
DBU: 1,8-diazabicyclo[5.4.0]undec-7-ene
(i-Pr)₂NEt, DIPEA: N,N-diisopropylethylamine
NMM: 4-methylmorpholine
Et₃N: triethylamine
THF: tetrahydrofuran
TBAF: Tetrabutylammonium fluoride
DMA: N,N-Dimethylacetamide
4-DMAP: 4-Dimethylaminopyridine
TBAI: Tetrabutylammonium iodide
CDI: N,N′-Carbonyldiimidazole
DCC: N,N′-Dicyclohexylcarbodiimide
DME: 1,2-Dimethoxyethane
TFAA: Trifluoroacetic anhydride
LAH, LiAlH₄: Lithium aluminum hydride
TBTU: O-(Benzotriazol-1-yl)-N,N,N′,N′-tetramethyluronium tetrafluoroborate
HATU: 1-[Bis(dimethylamino)methylene]-1H-1,2,3-triazolo[4,5-b]pyridinium 3-oxid hexafluorophosphate
DMF: N,N-Dimethylformamide
Aq.: aqueous
RT: room temperature

### General Synthetic Procedures

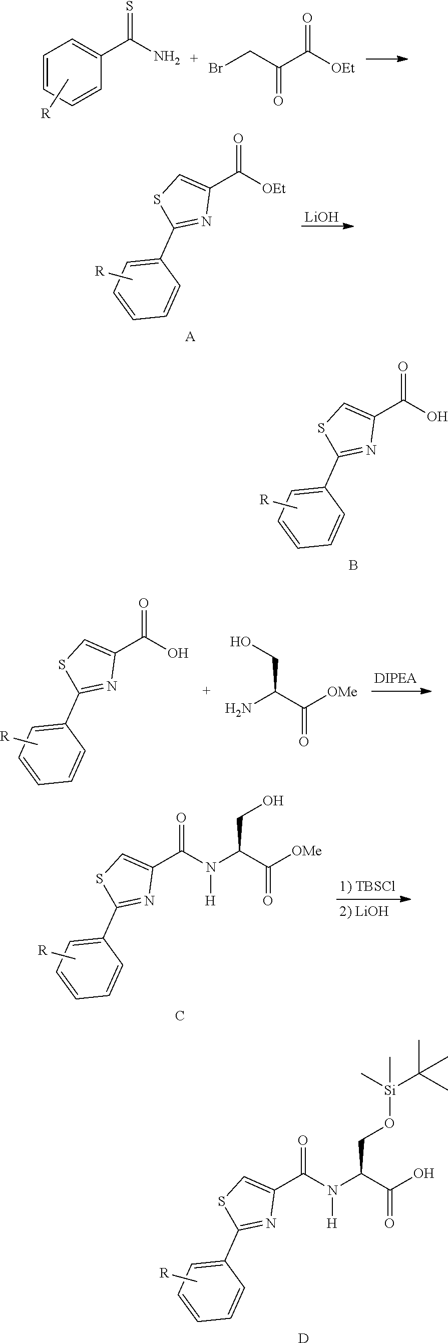

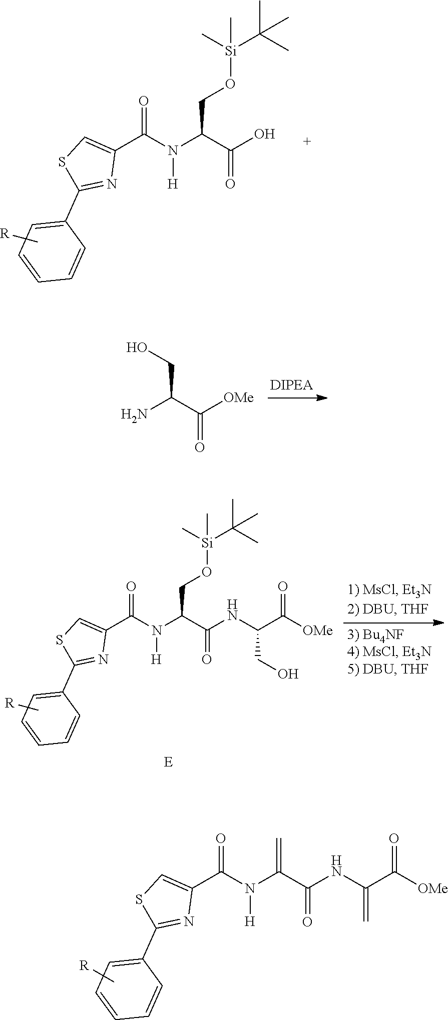

### General Procedures

Example A: The thioamide (1.0 eq.) was dissolved in ethanol (0.5M solution). Ethyl bromopyruvate (1.1 eq.) was added and the mixture was heated to 80° C. until the reaction was complete by TLC analysis. The mixture was cooled to RT and concentrated. The residue was suspended in saturated aqueous sodium bicarbonate (50 mL) and the mixture was extracted with ethyl acetate (3×20 mL). The combined organics were dried over magnesium sulfate, filtered, and concentrated. The crude residue was purified by silica gel chromatography to provide the Intermediate A.

Example B: Intermediate A (1.0 eq.) was dissolved in 4/1/1 THF/methanol/water. Lithium hydroxide (3.0 eq.) was added and the mixture was stirred at RT until the reaction was complete by TLC analysis. Water (25 mL) was added and the mixture was treated with 1N aq. HCl to pH=4. The resulting mixture was extracted with ethyl acetate (3×20 mL). The combined organics were dried over sodium sulfate, filtered, and concentrated to provide the desired Intermediate B.

Example C: Intermediate B (1.0 eq.) was dissolved in DCM. L-Serine methyl ester hydrochloride (1.2 eq.) was added, followed by N,N-diisopropylethylamine (2.0 eq.) and a coupling reagent (1.2 eq.). The mixture was stirred at RT until the reaction was complete by TLC analysis. Water (25 mL) was added and the mixture was extracted with DCM (3×15 mL). The combined organics were dried over sodium sulfate, filtered, and concentrated. The crude residue was purified by silica gel chromatography to provide Intermediate C.

Example D: Intermediate C was dissolved in DCM. Imidazole (1.2 eq.) was added followed by tert-butyldimethylchlorosilane (1.2 eq.). The reaction was stirred at RT until complete by TLC analysis. Water ((25 mL) was added and the mixture was extracted with DCM (3×15 mL). The combined organics were dried over sodium sulfate, filtered, and concentrated. The crude residue was purified by silica gel chromatography. The isolated compound was dissolved in 4/1/1 THF/methanol/water. Lithium hydroxide (3.0 eq.) was added and the mixture was stirred at RT until the reaction was complete as judged by TLC. Water (25 mL) was added and the solution was treated with 1N aq. HCl to pH=3. The mixture was extracted with ethyl acetate (3×15 mL). The combined organics were dried over sodium sulfate, filtered, and concentrated to provide Intermediate D.

Example E: Intermediate D (1.0 eq.) was dissolved in DCM and L-serine methyl ester hydrochloride (1.2 eq.) was added. N,N-Diisopropylethylamine (2.0 eq.) was added followed by a coupling reagent (1.2 eq.). The mixture was stirred at RT until the reaction was complete by TLC analysis. Water (25 mL) was added and the mixture was extracted with DCM (3×10 mL). The combined organics were dried over sodium sulfate, filtered, and concentrated. The crude residue was purified by silica gel chromatography to provide Intermediate E.

Example F: Intermediate E (1.0 eq.) was dissolved in DCM and cooled to 0° C. Triethylamine (1.5 eq.) was added followed by methanesulfonyl chloride (1.5 eq.). The mixture was stirred at 0° until complete by TLC analysis. Water (25 mL) was added and the mixture was extracted with DCM (3×15 mL). The combined organics were dried over sodium sulfate, filtered, and concentrated. The residue was dissolved in THF and cooled to 0° C. 1,8-Diazabicyclo[5.4.0]undec-7-ene (DBU) (1.2 eq.) was added and the mixture was stirred at 0° until the reaction was complete by TLC analysis. Water (25 mL) was added and the mixture was extracted with ethyl acetate (3×15 mL). The combined organics were dried over magnesium sulfate, filtered, and concentrated to provide the desired product. The residue (1.0 eq.) was dissolved in THF. Tetrabutylammonium fluoride (1.1 eq., 1.0M solution in THF) was added and the solution was stirred at RT until the reaction was complete by TLC analysis. Water (25 mL) was added and the mixture was extracted with ethyl acetate (3×15 mL). The combined organics were dried over magnesium sulfate, filtered, and concentrated. The isolated compound was dissolved in DCM and cooled to 0° C. Triethylamine (1.5 eq.) and methanesulfonyl chloride (1.5 eq.) were added. The mixture was stirred at RT until the reaction was complete by TLC analysis. Water (25 mL) was added and the mixture was extracted with DCM (3×15 mL). The combined organics were dried over sodium sulfate, filtered, and concentrated. The residue was dissolved in THF and cooled to 0° C. 1,8-Diazabicyclo[5.4.0]undec-7-ene (DBU, 1.2 eq.) was added and the mixture was stirred until complete by TLC analysis. Water (25 mL) was added and the mixture was extracted with ethyl acetate (3×15 mL). The combined organics were dried over magnesium sulfate, filtered, and concentrated. The crude residue was purified by silica gel chromatography to provide the desired product.

### Methyl N-(2-bromothiazole-4-carbonyl)-O-(tert-butyldimethylsilyl)-L-serinate

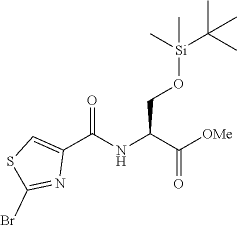 [SMILES: COC(=O)[C@H](CO[Si](C)(C)C(C)(C)C)NC(=O)c1csc(Br)n1]

2-Bromothiazole-4-carboxylic acid (1.807 g, 8.69 mmole) was dissolved in DCM (17 mL). L-Serine methyl ester hydrochloride (1.643 g, 10.6 mmole) was added followed by N,N-diisopropylethylamine (3.00 mL, 17.2 mmole) and pyBOP (5.455 g, 10.5 mmole). The mixture was stirred at RT for 90 minutes. Water (50 mL) was added and the two layers were separated. The aqueous layer was extracted with DCM (2×15 mL). The combined organics were dried over sodium sulfate, filtered, and concentrated. The crude residue was purified by silica gel chromatography (Isco CombiPrep, 24 g RediSep column, 50-90% ethyl acetate/hexane gradient) to provide methyl (2-bromothiazole-4-carbonyl)-L-serinate as a white solid (3.224 g).

¹H NMR (400 MHz, CDCl₃) δ 8.08 (s, 1H), 8.04-7.93 (m, 1H), 4.84 (dt, J=7.7, 3.8 Hz, 1H), 4.11 (dd, J=11.3, 4.0 Hz, 1H), 4.03 (dd, J=11.3, 3.6 Hz, 1H), 3.83 (s, 3H).

Methyl (2-bromothiazole-4-carbonyl)-L-serinate (3.224 g) was dissolved in DCM (20 mL). Imidazole (0.854 g, 12.5 mmole) was added followed by tert-butyldimethylchlorosilane (1.891 g, 12.5 mmole) and the mixture was stirred at RT for 45 minutes. Water (50 mL) was added and the two layers were separated. The aqueous layer was extracted with DCM (2×15 mL). The combined organics were dried over sodium sulfate, filtered, and concentrated. The crude product was purified by silica gel chromatography (Isco CombiPrep, 40 g Silicycle column, 10-40% ethyl acetate/hexane gradient) to provide methyl N-(2-bromothiazole-4-carbonyl)-O-(tert-butyldimethylsilyl)-L-serinate as a pale yellow oil (3.100 g, 84%).

¹H NMR (400 MHz, CDCl₃) δ 8.06 (s, 1H), 7.99-7.87 (m, 1H), 4.80 (dt, J=8.6, 3.1 Hz, 1H), 4.17 (dd, J=10.1, 2.9 Hz, 1H), 3.91 (dd, J=10.1, 3.5 Hz, 1H), 3.78 (s, 3H), 0.90 (s, 9H), 0.06 (s, 3H), 0.05 (s, 3H).

### Compound Synthesis

### Compound 1: Tert-butyl 4-(4-((3-((3-methoxy-3-oxoprop-1-en-2-yl)amino)-3-oxoprop-1-en-2-yl)carbamoyl)thiazol-2-yl)piperidine-1-carboxylate

### Tert-butyl 4-carbamothioylpiperidine-1-carboxylate (1.1)

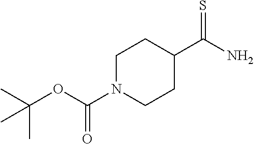 [SMILES: CC(C)(C)OC(=O)N1CCC(C(N)=S)CC1]

1-N-Boc-4-cyanopiperidine (2.005 g, 9.53 mmole) was dissolved in pyridine (10 mL). Triethylamine (1.50 mL, 10.7 mmole) was added followed by ammonium sulfide (40% aqueous solution, 1.80 mL, 10.5 mmole). The mixture was heated to 50° C. for 5 hours, then was cooled to RT and concentrated. The residue was dissolved in ethyl acetate (50 mL). The solution was washed with 1N aq. HCl (2×25 mL). The organics were dried over magnesium sulfate, filtered, and concentrated. The crude residue was purified by silica gel chromatography (Isco CombiPrep, 25 g Silicycle column, 30-60% ethyl acetate/hexane gradient) to provide tert-butyl 4-carbamothioylpiperidine-1-carboxylate as a white solid (1.174 g, 50%).

¹H NMR (400 MHz, Chloroform-d) δ 7.51 (s, 1H), 6.94 (s, 1H), 4.18 (d, J=37.0 Hz, 2H), 2.82-2.62 (m, 3H), 1.96-1.83 (m, 2H), 1.72 (dtd, J=13.2, 12.3, 4.4 Hz, 2H), 1.46 (s, 9H).

### Ethyl 2-(1-(tert-butoxycarbonyl)piperidin-4-yl)thiazole-4-carboxylate (1.2)

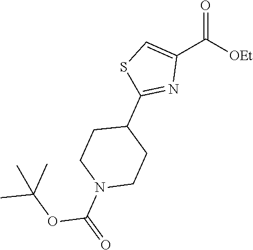 [SMILES: CCOC(=O)c1csc(C2CCN(C(=O)OC(C)(C)C)CC2)n1]

Tert-butyl 4-carbamothioylpiperidine-1-carboxylate (1.174 g, 4.80 mmole) was dissolved in 1,4-dioxane (24 mL) and cooled to 0° C. Potassium bicarbonate (3.868 g, 38.6 mmole) was added followed by ethyl bromopyruvate (1.80 mL, 14.3 mmole). The mixture was stirred at 0° for 4 hours, then was warmed to RT and stirred overnight. The slurry was concentrated to dryness and dissolved in a mixture of water (25 mL) and ethyl acetate (25 mL). The two layers were separated. The organics were washed with sat. aq. sodium chloride (1×25 mL), dried over magnesium sulfate, filtered, and concentrated. The residue was dissolved in 1,4-dioxane (24 mL) and cooled to 0° C. Pyridine (3.10 mL, 38.3 mmole) was added followed by trifluoroacetic anhydride (2.70 mL, 19.4 mmole). The mixture was stirred at 0° for 3 hours, then was warmed to RT and stirred for 2 hours. Triethylamine (16 mL) was added and the mixture was concentrated. The residue was dissolved in ethyl acetate (40 mL) and washed sequentially with 0.5N aq. HCl (2×50 mL), sat. aq. sodium bicarbonate (1×25 mL), and sat. aq. sodium chloride (1×25 mL). The organics were dried over magnesium sulfate, filtered, and concentrated. The crude residue was purified by silica gel chromatography (Isco CombiPrep, 25 g Silicycle column, 15-35% ethyl acetate/hexane gradient) to provide ethyl 2-(1-(tert-butoxycarbonyl)piperidin-4-yl)thiazole-4-carboxylate as a tan solid (1.704 g, quant.).

¹H NMR (400 MHz, Chloroform-d) δ 8.08 (s, 1H), 4.42 (q, J=7.1 Hz, 2H), 4.30-4.16 (m, 2H), 3.26 (tt, J=11.8, 3.7 Hz, 1H), 2.85 (t, J=13.0 Hz, 2H), 2.18-2.07 (m, 2H), 1.80-1.65 (m, 2H), 1.47 (s, 9H), 1.40 (t, J=7.1 Hz, 3H).

### 2-(1-(Tert-butoxycarbonyl)piperidin-4-yl)thiazole-4-carboxylic Acid (1.3)

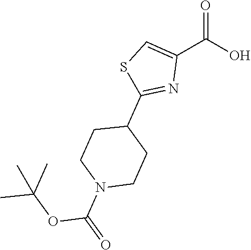 [SMILES: CC(C)(C)OC(=O)N1CCC(c2nc(C(=O)O)cs2)CC1]

Ethyl 2-(1-(tert-butoxycarbonyl)piperidin-4-yl)thiazole-4-carboxylate (1.704 g, 5.01 mmole) was dissolved in 4/1/1 THF/methanol/water (15 mL). Lithium hydroxide (0.484 g, 20.2 mmole) was added and the mixture was stirred at RT for 2 hours, then was concentrated to the aqueous layer. The solid was dissolved in water (200 mL) and the solution was treated with 1N aq. HCl to pH=3. The mixture was extracted with ethyl acetate (5×20 mL) and the combined organics were dried over sodium sulfate, filtered, and concentrated to provide 2-(1-(tert-butoxycarbonyl)piperidin-4-yl)thiazole-4-carboxylic acid as a golden solid (1.451 g, 93%).

1H NMR (400 MHz, Chloroform-d) δ 8.20 (s, 1H), 4.23 (s, 2H), 3.23 (tt, J=11.7, 3.8 Hz, 1H), 2.88 (t, J=12.7 Hz, 2H), 2.20-2.08 (m, 2H), 1.84-1.67 (m, 2H), 1.48 (s, 9H).

### Tert-butyl (S)-4-(4-((3-hydroxy-1-methoxy-1-oxopropan-2-yl)carbamoyl)thiazol-2-yl)piperidine-1-carboxylate (1.4)

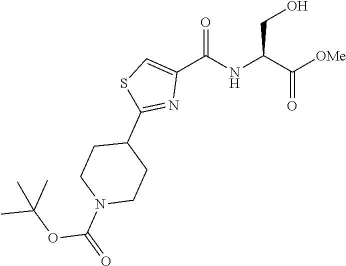 [SMILES: COC(=O)[C@H](CO)NC(=O)c1csc(C2CCN(C(=O)OC(C)(C)C)CC2)n1]

2-(1-(Tert-butoxycarbonyl)piperidin-4-yl)thiazole-4-carboxylic acid (1.451 g, 4.64 mmole) was dissolved in DCM (9 mL). L-Serine methyl ester hydrochloride (0.870 g, 5.59 mmole) was added, followed sequentially by N,N-diisopropylethylamine (2.40 mL, 13.8 mmole) and BOP reagent (2.475 g, 5.60 mmole). The mixture was stirred at RT overnight and water (50 mL) was added. The two layers were separated and the aqueous layer was extracted with DCM (3×20 mL). The combined organics were dried over sodium sulfate, filtered, and concentrated. The crude residue was purified by silica gel chromatography to provide tert-butyl (S)-4-(4-((3-hydroxy-1-methoxy-1-oxopropan-2-yl)carbamoyl)thiazol-2-yl)piperidine-1-carboxylate as a thick, yellow gel (1.763 g, 92%).

¹H NMR (400 MHz, Chloroform-d) δ 8.12 (d, J=7.5 Hz, 1H), 8.02 (s, 1H), 4.84 (dt, J=7.6, 3.8 Hz, 1H), 4.21 (s, 2H), 4.11-4.01 (m, 2H), 3.83 (s, 3H), 3.14 (tt, J=11.6, 3.7 Hz, 1H), 2.89 (t, J=12.4 Hz, 2H), 2.65 (t, J=6.1 Hz, 1H), 2.17-2.07 (m, 2H), 1.75 (qd, J=12.4, 4.3 Hz, 2H), 1.48 (s, 9H).

### Tert-butyl 4-(4-((3-methoxy-3-oxoprop1-en-2-yl)carbamoyl)thiazol-2-yl)piperidine-1-carboxylate (1.5)

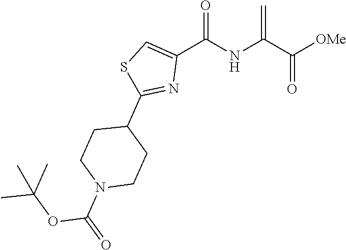 [SMILES: C=C(NC(=O)c1csc(C2CCN(C(=O)OC(C)(C)C)CC2)n1)C(=O)OC]

Tert-butyl (S)-4-(4-((3-hydroxy-1-methoxy-1-oxopropan-2-yl)carbamoyl)thiazol-2-yl)piperidine-1-carboxylate (1.763 g, 4.26 mmole) was dissolved in DCM (9 mL) and cooled to 0° C. Methanesulfonyl chloride (0.365 mL, 4.72 mmole) was added followed by dropwise addition of triethylamine (0.700 mL, 4.99 mmole). The mixture was stirred at 0° for 60 minutes and water (50 mL) was added. The two layers were separated and the aqueous layer was extracted with DCM (2×15 mL). The combined organics were dried over sodium sulfate, filtered, and concentrated. The residue was dissolved in THF (9 mL) and cooled to 0° C. 1,8-Diazabicyclo[5.4.0]undec-7-ene (0.700 mL, 4.68 mmole) was added dropwise and the solution was stirred at 0° for 90 minutes, then at RT overnight. Water (50 mL) was added and the mixture was extracted with ethyl acetate (3×15 mL). The combined organics were dried over magnesium sulfate, filtered, and concentrated. The crude residue was purified by silica gel chromatography to provide tert-butyl 4-(4-((3-methoxy-3-oxoprop1-en-2-yl)carbamoyl)thiazol-2-yl)piperidine-1-carboxylate as a white solid (1.227 g, 73%).

¹H NMR (400 MHz, Chloroform-d) δ 9.69 (s, 1H), 8.05 (s, 1H), 6.76 (s, 1H), 5.98 (d, J=1.6 Hz, 1H), 4.32-4.13 (m, 2H), 3.90 (s, 3H), 3.16 (tt, J=11.6, 3.8 Hz, 1H), 2.90 (t, J=12.7 Hz, 2H), 2.21-2.08 (m, 2H), 1.76 (dtd, J=13.1, 11.8, 4.3 Hz, 2H).

### 2-(2-(1-(Tert-butoxycarbonyl)piperidin-4-yl)thiazole-4-carboxamido)acrylic Acid (1.6)

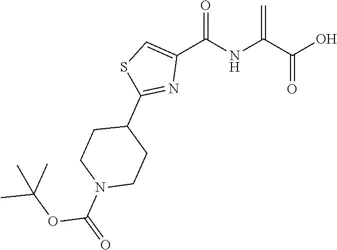 [SMILES: C=C(NC(=O)c1csc(C2CCN(C(=O)OC(C)(C)C)CC2)n1)C(=O)O]

The procedure described in example 1.3 was used to convert tert-butyl 4-(4-((3-methoxy-3-oxoprop1-en-2-yl)carbamoyl)thiazol-2-yl)piperidine-1-carboxylate (1.227 g, 3.10 mmole) to 2-(2-(1-(tert-butoxycarbonyl)piperidin-4-yl)thiazole-4-carboxamido)acrylic acid as a pale yellow solid (1.130 g, 96%).

¹H NMR (400 MHz, DMSO-d₆) δ 9.65 (s, 1H), 8.36 (s, 1H), 6.52 (s, 1H), 5.82 (d, J=1.5 Hz, 1H), 4.03 (d, J=13.1 Hz, 2H), 3.38-3.24 (m, 1H), 2.09-2.01 (m, 2H), 1.66-1.50 (m, 2H), 1.41 (s, 9H).

### Tert-butyl (S)-4-(4-((3-((3-hydroxy-1-methoxy-1-oxopropan-2-yl)amino)-3-oxoprop-1-en-2-yl)carbamoyl)thiazol-2-yl)piperidine-1-carboxylate (1.7)

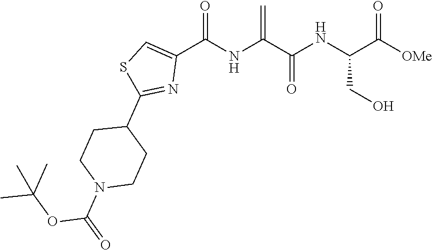 [SMILES: C=C(NC(=O)c1csc(C2CCN(C(=O)OC(C)(C)C)CC2)n1)C(=O)N[C@@H](CO)C(=O)OC]

2-(2-(1-(Tert-butoxycarbonyl)piperidin-4-yl)thiazole-4-carboxamido)acrylic acid (0.706 g, 1.85 mmole) was suspended in DCM (6 mL). L-Serine methyl ester hydrochloride (0.408 g, 2.62 mmole) was added followed by N,N-diisopropylethylamine (0.970 mL, 5.57 mmole). Propylphosphonic acid anhydride (50% solution in 2-MeTHF, 0.650 mL, 2.22 mmole) was added and the mixture was stirred at RT for 90 minutes. The solution was loaded onto a silica gel column and purified by silica gel chromatography (Isco CombiPrep, 25 g Silicycle column, 60-90% ethyl acetate/hexane gradient) to provide tert-butyl (S)-4-(4-((3-((3-hydroxy-1-methoxy-1-oxopropan-2-yl)amino)-3-oxoprop-1-en-2-yl)carbamoyl)thiazol-2-yl)piperidine-1-carboxylate as a white solid (0.310 g, 35%).

¹H NMR (400 MHz, Chloroform-d) δ 9.85 (s, 1H), 8.05 (s, 1H), 7.08 (d, J=7.3 Hz, 1H), 6.64 (d, J=1.9 Hz, 1H), 5.48 (t, J=1.6 Hz, 1H), 4.76 (dt, J=7.0, 3.4 Hz, 1H), 4.20 (s, 2H), 4.12-4.00 (m, 2H), 3.84 (s, 3H), 3.14 (tq, J=11.8, 4.1 Hz, 1H), 2.88 (t, J=12.7 Hz, 2H), 2.49 (s, 1H), 2.21-2.08 (m, 2H), 1.75 (dtd, J=13.3, 11.8, 4.3 Hz, 2H), 1.48 (s, 9H).

### Tert-butyl 4-(4-((3-((3-methoxy-3-oxoprop-1-en-2-yl)amino)-3-oxoprop-1-en-2-yl)carbamoyl)thiazol-2-yl)piperidine-1-carboxylate (1)

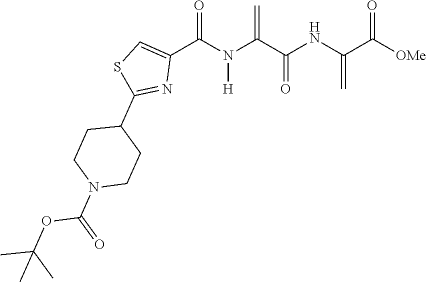 [SMILES: C=C(NC(=O)c1csc(C2CCN(C(=O)OC(C)(C)C)CC2)n1)C(=O)NC(=C)C(=O)OC]

Tert-butyl (S)-4-(4-((3-((3-hydroxy-1-methoxy-1-oxopropan-2-yl)amino)-3-oxoprop-1-en-2-yl)carbamoyl)thiazol-2-yl)piperidine-1-carboxylate (0.310 g, 0.808 mmole) was dissolved in DCM (1.6 mL) and cooled to 0° C. Methanesulfonyl chloride (0.070 mL, 0.904 mmole) was added followed by triethylamine (0.125 mL, 0.892 mmole). The solution was stirred at 0° for 90 minutes and water (25 mL) was added. The mixture was extracted with DCM (3×15 mL). The combined organics were dried over sodium sulfate, filtered, and concentrated. The residue was dissolved in THF (1.6 mL) and cooled to 0° C. 1,8-Diazabicyclo[5.4.0]undec-7-ene (0.145 mL, 0.970 mmole) was added and the mixture was stirred at 0° for 60 minutes. Water (25 mL) was added and the mixture was extracted with ethyl acetate (3×15 mL). The combined organics were dried over magnesium sulfate, filtered, and concentrated. The crude residue was purified by silica gel chromatography (Isco CombiPrep, 12 g RediSep Gold column, 20-40% ethyl acetate/hexane gradient) to provide tert-butyl 4-(4-((3-((3-methoxy-3-oxoprop-1-en-2-yl)amino)-3-oxoprop-1-en-2-yl)carbamoyl)thiazol-2-yl)piperidine-1-carboxylate as a white solid (0.249 g, 66%).

1H NMR (400 MHz, Chloroform-d) δ 9.89 (s, 1H), 8.54 (s, 1H), 8.06 (s, 1H), 6.75 (d, J=2.2 Hz, 1H), 6.69 (s, 1H), 6.02 (d, J=1.1 Hz, 1H), 5.47 (t, J=1.9 Hz, 1H), 4.22 (s, 2H), 3.90 (s, 3H), 3.17 (tt, J=11.6, 3.7 Hz, 1H), 2.89 (t, J=12.7 Hz, 2H), 2.14 (d, J=13.1 Hz, 2H), 1.84-1.68 (m, 2H), 1.49 (s, 9H).

### Compound 2: Methyl 2-(2-(2-(6-((tert-butoxycarbonyl)amino)pyridine-3-yl)thiazole-4-carboxamido)acrylamido)acrylate

### Tert-butyl 2-(tert-butoxycarbonyl)amino-5-cyanopyridine Carbamate (2.1)

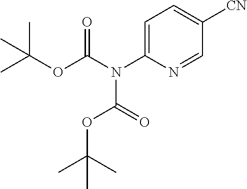 [SMILES: CC(C)(C)OC(=O)N(C(=O)OC(C)(C)C)c1ccc(C#N)cn1]

2-Amino-5-cyanopyridine (3.010 g, 25.3 mmole) was dissolved in DCM (30 mL). Triethylamine (7.00 mL, 49.9 mmole) and 4-dimethylaminopyridine (0.305 g, 0.250 mole) were added. Di-tert-butyl dicarbonate (11.013 g, 50.5 mmole) was added and the solution was stirred at RT for 24 hours. Water (100 mL) was added and the two layers were separated. The aqueous layer was extracted with DCM (2×30 mL). The combined organics were washed with sat. aq. sodium chloride (1×50 mL), then dried over sodium sulfate, filtered, and concentrated. The crude residue was purified by silica gel chromatography (Isco CombiPrep, 40 g Silicycle column, 10-25% ethyl acetate/hexane gradient) to provide tert-butyl 2-(tert-butoxycarbonyl)amino-5-cyanopyridine carbamate as a white solid (7.185 g, 89%).

¹H NMR (400 MHz, Chloroform-d) δ 8.66 (dd, J=2.3, 0.8 Hz, 1H), 7.95 (dd, J=8.6, 2.3 Hz, 1H), 7.67 (dd, J=8.6, 0.8 Hz, 1H), 1.51 (s, 18H).

### 2-((Bis-N-Boc)amino)-5-(thiocarboxamido)pyridine (2.2)

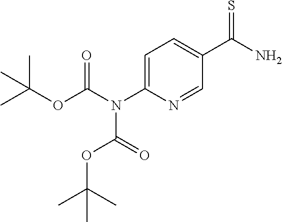 [SMILES: CC(C)(C)OC(=O)N(C(=O)OC(C)(C)C)c1ccc(C(N)=S)cn1]

Tert-butyl 2-(tert-butoxycarbonyl)amino-5-cyanopyridine carbamate (4.994 g, 15.6 mmole) was dissolved in pyridine (16 mL). Triethylamine (2.40 mL, 17.1 mmole) was added followed by ammonium sulfide (40% aqueous solution, 3.20 mL, 18.7 mmole) and the mixture was heated to 50° C. for 7 hours. The mixture was cooled to RT and concentrated. The residue was dissolved in ethyl acetate (50 mL) and washed with water (2×25 mL) and brine (1×25 mL). The organics were dried over magnesium sulfate, filtered, and concentrated. The crude residue was purified by silica gel chromatography (Isco CombiPrep, 40 g Silicycle column, 10-30% ethyl acetate/hexane gradient) to provide 2-((bis-N-Boc)amino)-5-(thiocarboxamido)pyridine as a yellow solid (6.353 g).

¹H NMR (400 MHz, Chloroform-d) δ 8.86 (dd, J=2.6, 0.8 Hz, 1H), 8.28 (dd, J=8.5, 2.6 Hz, 1H), 7.83-7.69 (m, 1H), 7.54 (s, 1H), 7.42 (dd, J=8.5, 0.8 Hz, 1H), 1.49 (s, 18H).

### Ethyl 2-(4-(bis(tert-butoxycarbonyl)amino)pyridine-3-yl)thiazole-4-carboxylate (2.3)

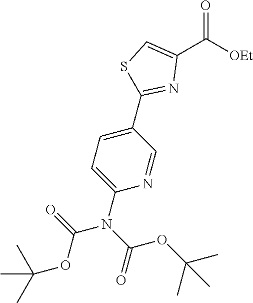 [SMILES: CCOC(=O)c1csc(-c2ccc(N(C(=O)OC(C)(C)C)C(=O)OC(C)(C)C)nc2)n1]

2-((Bis-N-Boc)amino)-5-(thiocarboxamido)pyridine (6.353 g, 18.0 mmole) was dissolved in 1,4-dioxane (90 mL) and cooled to 0° C. Potassium bicarbonate (14.574 g, 146 mmole) was added followed by slow addition of ethyl bromopyruvate (4.50 mL, 35.9 mmole). The mixture was stirred at 0° for 4 hours, then was warmed to RT and allowed to stir overnight. The mixture was concentrated and the residue was suspended in ethyl acetate (100 mL). The mixture was washed sequentially with water (2×50 mL) and sat. aq. sodium chloride (1×50 mL). The organics were dried over magnesium sulfate, filtered, and concentrated. The residue was dissolved in 1,4-dioxane (90 mL) and cooled to 0° C. Pyridine (11.6 mL, 143 mmole) was added followed by slow addition of trifluoroacetic anhydride (5.00 mL, 36.0 mmole). The dark red mixture was stirred at 0° for 3 hours, then at RT for 2 hours. Triethylamine (30 mL) was added and the mixture was concentrated. The residue was suspended in ethyl acetate (75 mL) and washed sequentially with 0.5N aq. HCl (3×50 mL) and sat. aq. Sodium bicarbonate (1×25 mL). The organics were dried over magnesium sulfate, filtered, and concentrated. The crude residue was purified by silica gel chromatography (Isco CombiPrep, 40 g Silicycle column, 10-30% ethyl acetate/hexane gradient) to provide ethyl 2-(4-(bis(tert-butoxycarbonyl)amino)pyridine-3-yl)thiazole-4-carboxylate as a yellow solid (6.302 g, 78%).

¹H NMR (400 MHz, Chloroform-d) δ 9.02 (dd, J=2.4, 0.8 Hz, 1H), 8.37 (dd, J=8.4, 2.5 Hz, 1H), 8.22 (s, 1H), 7.44 (dd, J=8.4, 0.8 Hz, 1H), 4.46 (qd, J=7.1, 3.9 Hz, 2H), 1.47 (s, 18H), 1.44 (td, J=7.1, 2.3 Hz, 3H).

### 2-(6-((Tert-butoxycarbonyl)amino)pyridine-3-yl)thiazole-4-carboxylic Acid (2.4)

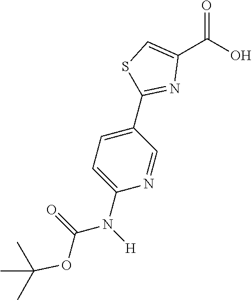 [SMILES: CC(C)(C)OC(=O)Nc1ccc(-c2nc(C(=O)O)cs2)cn1]

Using the procedure described for Example 1.3, ethyl 2-(4-(bis(tert-butoxycarbonyl)amino)pyridine-3-yl)thiazole-4-carboxylate (6.302 g, 14.0 mmole) was converted to 2-(6-((tert-butoxycarbonyl)amino)pyridine-3-yl)thiazole-4-carboxylic acid (3.323 g, 74%) as a white solid.

¹H NMR (400 MHz, DMSO-d₆) δ 10.20 (s, 1H), 8.83 (dd, J=2.5, 0.8 Hz, 1H), 8.49 (s, 1H), 8.29 (dd, J=8.8, 2.5 Hz, 1H), 7.97 (dd, J=8.8, 0.8 Hz, 1H), 1.49 (s, 9H).

### Methyl (2-(6-((tert-butyoxycarbonyl)amino)pyridine-3-yl)thiazole-4-carbonyl)-L-serinate (2.5)

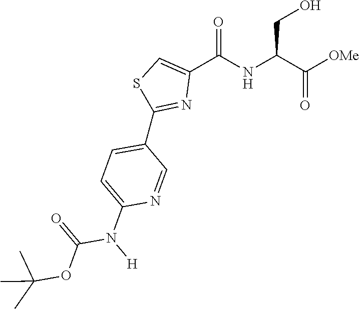 [SMILES: COC(=O)[C@H](CO)NC(=O)c1csc(-c2ccc(NC(=O)OC(C)(C)C)nc2)n1]

Using the procedure described for Example 1.4, 2-(6-((tert-butoxycarbonyl)amino)pyridine-3-yl)thiazole-4-carboxylic acid (3.323 g, 10.3 mmole) was converted to methyl (2-(6-((tert-butyoxycarbonyl)amino)pyridine-3-yl)thiazole-4-carbonyl)-L-serinate (6.065 g) as a white solid. 1H NMR (400 MHz, Chloroform-d) δ 8.79 (dd, J=2.3, 0.8 Hz, 1H), 8.62 (s, 1H), 8.25-8.14 (m, 2H), 8.14-8.05 (m, 2H), 4.88 (dt, J=7.6, 3.7 Hz, 1H), 4.20-4.08 (m, 2H), 3.85 (s, 3H), 1.57 (s, 9H).

### N-(2-(6-((Tert-butoxycarbonyl)amino)pyridine-3-yl)thiazole-4-carbonyl)-O-(tert-butyldimethylsilyl)-L-serine (2.6)

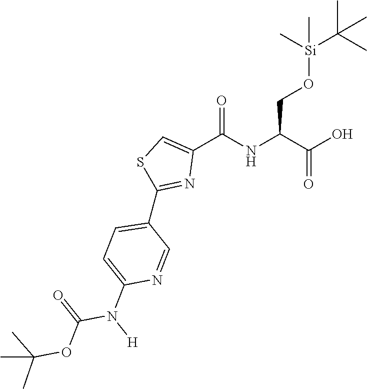 [SMILES: CC(C)(C)OC(=O)Nc1ccc(-c2nc(C(=O)N[C@@H](CO[Si](C)(C)C(C)(C)C)C(=O)O)cs2)cn1]

Methyl 2-(6-((bis(tert-butoxycarbonyl)amino)pyridine-3-yl)thiazole-4-carbonyl)-L-serinate (1.723 g, 3.30 mmole) was dissolved in DMF (6.5 mL). Tert-butyldimethylchlorosilane (0.551 g, 3.66 mmole) was added followed by imidazole (0.274 g, 4.02 mmole) and the solution was stirred at RT for 90 minutes. Water (50 mL) was added and the mixture was extracted with diethyl ether (3×20 mL). The combined organics were dried over magnesium sulfate, filtered, and concentrated. The crude residue was purified by silica gel chromatography to provide methyl O-(tert-butyldimethylsilyl)-N-(6-((bis(tert-butoxycarbonyl)amino)pyridine-3-yl)thiazole-4-carbonyl)-L-serinate as a colorless oil (1.711 g, 81%).
Methyl O-(tert-butyldimethylsilyl)-N-(6-((bis(tert-butoxycarbonyl)amino)pyridine-3-yl)thiazole-4-carbonyl)-L-serinate (1.711 g, 2.69 mmole) was dissolved in 4/1/1 THF/methanol/water (9 mL). Lithium hydroxide (0.322 g, 13.4 mmole) was added and the mixture was stirred at RT for 2 hours, then was concentrated to the aqueous layer. The residue was suspended in water (50 mL) and the mixture was treated with 1N aq. HCl to pH=3. The resulting mixture was extracted with ethyl acetate (4×20 mL). The combined organics were dried over sodium sulfate, filtered, and concentrated to provide N-(2-(6-((tert-butoxycarbonyl)amino)pyridine-3-yl)thiazole-4-carbonyl)-O-(tert-butyldimethylsilyl)-L-serine (4.856 g, 100%) as a white foamy solid.

¹H NMR (400 MHz, DMSO-d₆) δ 10.28 (s, 1H), 8.92 (dd, J=2.5, 0.8 Hz, 1H), 8.43 (s, 1H), 8.33 (dd, J=8.8, 2.5 Hz, 1H), 8.22 (d, J=8.6 Hz, 1H), 8.01 (dd, J=8.8, 0.8 Hz, 1H), 4.64 (dt, J=8.6, 3.6 Hz, 1H), 4.15 (dd, J=10.3, 3.6 Hz, 1H), 4.00 (dd, J=10.3, 3.7 Hz, 1H), 1.54 (s, 9H), 0.92 (s, 9H), 0.10 (s, 3H), 0.08 (s, 3H).

### Methyl N-(2-(6-((tert-butoxycarbonyl)amino)pyridine-3-yl)thiazole-4-carbonyl)-O-(tert-butyldimethylsilyl)-L-seryl-L-serinate (2.7)

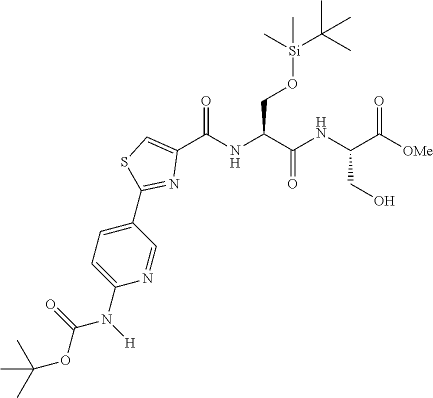 [SMILES: COC(=O)[C@H](CO)NC(=O)[C@H](CO[Si](C)(C)C(C)(C)C)NC(=O)c1csc(-c2ccc(NC(=O)OC(C)(C)C)nc2)n1]

Using the general procedure described for Example 1.4, N-(2-(6-((tert-butoxycarbonyl)amino)pyridine-3-yl)thiazole-4-carbonyl)-O-(tert-butyldimethylsilyl)-L-serine (3.010 g, 5.76 mmole) was converted to methyl N-(2-(6-((tert-butoxycarbonyl)amino)pyridine-3-yl)thiazole-4-carbonyl)-O-(tert-butyldimethylsilyl)-L-seryl-L-serinate (3.276 g, 91%) as a white solid.

¹H NMR (400 MHz, Chloroform-d) δ 8.82-8.75 (m, 1H), 8.25-8.16 (m, 2H), 8.12 (d, J=5.6 Hz, 1H), 8.06 (dt, J=8.8, 1.1 Hz, 1H), 7.85 (d, J=19.8 Hz, 1H), 7.46 (d, J=7.3 Hz, 1H), 4.74-4.61 (m, 2H), 4.20 (dd, J=9.8, 4.1 Hz, 1H), 4.06-3.93 (m, 2H), 3.86 (ddd, J=9.8, 8.3, 6.3 Hz, 1H), 3.78 (s, 3H), 1.56 (s, 9H), 0.94 (s, 9H), 0.14 (s, 3H), 0.13 (s, 3H).

### Methyl 2-(2-(2-(6-((tert-butoxycarbonyl)amino)pyridine-3-yl)thiazole-4-carboxamido)acrylamido)acrylate (2)

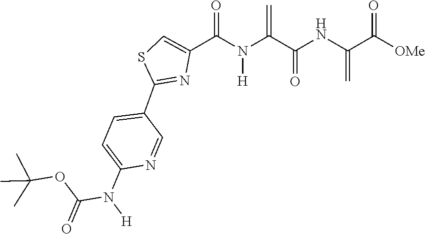 [SMILES: C=C(NC(=O)c1csc(-c2ccc(NC(=O)OC(C)(C)C)nc2)n1)C(=O)NC(=C)C(=O)OC]

Methyl N-(2-(6-((tert-butoxycarbonyl)amino)pyridine-3-yl)thiazole-4-carbonyl)-O-(tert-butyldimethylsilyl)-L-seryl-L-serinate (1.148 g, 1.84 mmole) was dissolved in DCM (4 mL) and cooled to 0° C. Methanesulfonyl chloride (0.160 mL, 2.07 mmole) was added followed by triethylamine (0.290 mL, 2.07 mmole). The mixture was stirred at 0° for 90 minutes and water (50 mL) was added. The mixture was extracted with DCM (3×15 mL). The combined organics were dried over sodium sulfate, filtered, and concentrated. The residue was dissolved in THF (4 mL) and cooled to 0° C. 1,8-Diazabicyclo[5.4.0]undec-7-end (0.300 mL, 2.01 mmole) was added and the solution was stirred at 0° for 2 hours, then was warmed to RT and stirred overnight. Water (50 mL) was added and the mixture was extracted with ethyl acetate (3×15 mL). The combined organics were dried over magnesium sulfate, filtered, and concentrated. The crude residue was purified by silica gel chromatography to provide methyl (S)-2-(2-(2-(6-((tert-butoxycarbonyl)amino)pyridin-3-yl)thiazole-4-carboxamido)-3-((tert-butyldimethylsilyl)oxy)propanamido)acrylate as a white solid (0.838 g, 75%).
Methyl (S)-2-(2-(2-(6-((tert-butoxycarbonyl)amino)pyridin-3-yl)thiazole-4-carboxamido)-3-((tert-butyldimethylsilyl)oxy)propanamido)acrylate (0.838 g, 1.38 mmole) was dissolved in THF (2.8 mL) and cooled to 0° C. Tetrabutylammonium fluoride (1.0M solution in THF, 1.55 mL, 1.55 mmole) was added and the solution was stirred at 0° for 4 hours. Water (50 mL) was added and the two layers were extracted with ethyl acetate (3×15 mL). The combined organics were dried over magnesium sulfate, filtered, and concentrated. The crude residue was purified by silica gel chromatography to provide the desired product as a white solid (0.558 g, 82%). Using the dehydration procedure described above, methyl (S)-2-(2-(2-(6-((tert-butoxycarbonyl)amino)pyridine-3-yl)thiazole-4-carboxamido)-3-hydroxypropanamido)acrylate (0.558 g, 1.14 mmole) was converted to methyl 2-(2-(2-(6-((tert-butoxycarbonyl)amino)pyridine-3-yl)thiazole-4-carboxamido)acrylamide)acrylate (0.162 g, 19%) as a white solid.

¹H NMR (400 MHz, Chloroform-d) δ 10.03-9.93 (m, 1H), 8.91 (dd, J=2.4, 0.8 Hz, 1H), 8.55 (s, 1H), 8.32 (s, 1H), 8.25 (ddd, J=8.8, 2.4, 0.5 Hz, 1H), 8.15 (s, 1H), 8.10 (dd, J=8.8, 0.8 Hz, 1H), 6.78 (d, J=2.3 Hz, 1H), 6.71 (s, 1H), 6.03 (d, J=1.3 Hz, 1H), 5.51 (t, J=1.9 Hz, 1H), 3.91 (s, 3H), 1.56 (s, 9H).

### Compound 3: Tert-butyl 3-(4-((3-((3-methoxy-3-oxoprop-1-en-2-yl)amino)-3-oxoprop-1-en-2-yl)carbamoyl)thiazol-2-yl)piperidine-1-carboxylate

### Tert-butyl 3-(4-(((S)-3-((tert-butyldimethylsilyl)oxy)-1-methoxy-1-oxopropan-2-yl)carbamoyl)thiazol-2-yl)piperidine-1-carboxylate (3.1)

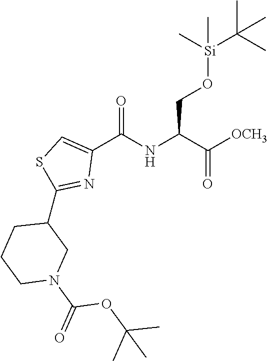 [SMILES: COC(=O)[C@H](CO[Si](C)(C)C(C)(C)C)NC(=O)c1csc(C2CCCN(C(=O)OC(C)(C)C)C2)n1]

Using the procedure described for Example 1, 1-N-Boc-3-cyanopiperidine (2.505 g, 11.9 mmole) was converted to tert-butyl 3-(4-(((S)-3-((tert-butyldimethylsilyl)oxy)-1-methoxy-1-oxopropan-2-yl)carbamoyl)thiazol-2-yl)piperidine-1-carboxylate (1.002 g).

¹H NMR (400 MHz, Chloroform-d) δ 8.08 (d, J=8.7 Hz, 1H), 8.01 (s, 1H), 4.88-4.78 (m, 1H), 4.30 (q, J=7.1 Hz, 1H), 4.23-4.14 (m, 1H), 4.01 (d, J=13.3 Hz, 1H), 3.92 (dd, J=10.1, 3.5 Hz, 1H), 3.78 (s, 3H), 3.14 (dt, J=10.6, 6.1 Hz, 2H), 2.97-2.82 (m, 1H), 2.27-2.15 (m, 1H), 1.90-1.73 (m, 2H), 1.63 (s, 1H), 1.48 (s, 9H), 0.89 (s, 9H), 0.06 (s, 3H), 0.04 (s, 3H).

### N-(2-(1-(Tert-butoxycarbonyl)piperidine-3-yl)thiazole-4-carbonyl)-O-(tert-butyldimethylsilyl)-L-serine (3.2)

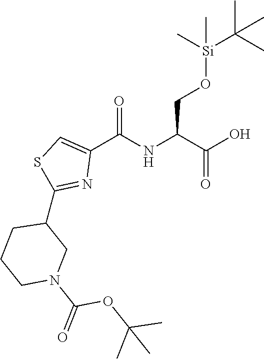 [SMILES: CC(C)(C)OC(=O)N1CCCC(c2nc(C(=O)N[C@@H](CO[Si](C)(C)C(C)(C)C)C(=O)O)cs2)C1]

Using the procedure described for Example 1.3, tert-butyl 3-(4-(((S)-3-((tert-butyldimethylsilyl)oxy)-1-methoxy-1-oxopropan-2-yl)carbamoyl)thiazol-2-yl)piperidine-1-carboxylate (1.002 g, 1.90 mmole) was converted to N-(2-(1-(tert-butoxycarbonyl)piperidine-3-yl)thiazole-4-carbonyl)-O-(tert-butyldimethylsilyl)-L-serine (1.041 g) as a colorless oil.

¹H NMR (400 MHz, Chloroform-d) δ 8.13 (d, J=8.2 Hz, 1H), 8.06 (d, J=7.4 Hz, 1H), 4.90-4.79 (m, 1H), 4.41-4.27 (m, 1H), 4.24 (dd, J=10.1, 3.0 Hz, 1H), 4.08-3.98 (m, 1H), 3.95 (dd, J=10.1, 4.0 Hz, 1H), 3.21-3.00 (m, 2H), 2.98-2.83 (m, 1H), 2.28-2.14 (m, 1H), 1.89-1.72 (m, 2H), 1.60 (d, J=20.6 Hz, 1H), 1.47 (s, 10H), 0.90 (s, 9H), 0.08 (d, J=2.3 Hz, 6H).

### Tert-butyl 3-(4-(((4S,7S)-4-(hydroxymethyl)-10,10,11,11-tetramethyl-3,6-dioxo-2,9-dioxa-5-aza-10-siladodecan-7-yl)carbamoyl)thiazol-2-yl)piperidine-1-carboxylate (3.3)

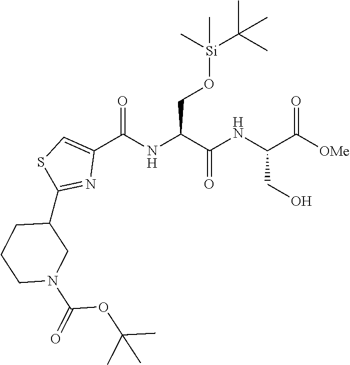 [SMILES: COC(=O)[C@H](CO)NC(=O)[C@H](CO[Si](C)(C)C(C)(C)C)NC(=O)c1csc(C2CCCN(C(=O)OC(C)(C)C)C2)n1]

Using the general procedure described for Example 1.4, N-(2-(1-(tert-butoxycarbonyl)piperidine-3-yl)thiazole-4-carbonyl)-O-(tert-butyldimethylsilyl)-L-serine (1.041 g, 2.03 mmole) was converted to tert-butyl 3-(4-(((4S,7S)-4-(hydroxymethyl)-10,10,11,11-tetramethyl-3,6-dioxo-2,9-dioxa-5-aza-10-siladodecan-7-yl)carbamoyl)thiazol-2-yl)piperidine-1-carboxylate (0.728 g, 58%) as a colorless oil.

¹H NMR (400 MHz, Chloroform-d) δ 8.12 (d, J=7.0 Hz, 1H), 8.06-7.98 (m, 1H), 7.44 (d, J=7.2 Hz, 1H), 4.68 (dt, J=7.3, 3.7 Hz, 1H), 4.66-4.57 (m, 1H), 4.21-4.15 (m, 1H), 3.99 (t, J=7.7 Hz, 3H), 3.84 (dtd, J=9.8, 6.9, 1.2 Hz, 1H), 3.78 (d, J=4.6 Hz, 3H), 3.14 (s, 2H), 2.99-2.83 (m, 2H), 2.28-2.13 (m, 1H), 1.90-1.73 (m, 3H), 1.60 (t, J=11.8 Hz, 1H), 1.47 (d, J=3.2 Hz, 9H), 0.92 (s, 9H), 0.13 (s, 3H), 0.12 (s, 3H).

### Tert-butyl 3-(4-((3-((3-methoxy-3-oxoprop-1-en-2-yl)amino)-3-oxoprop-1-en-2-yl)carbamoyl)thiazol-2-yl)piperidine-1-carboxylate (3)

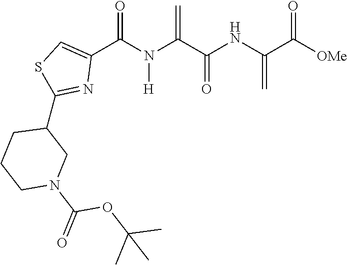 [SMILES: C=C(NC(=O)c1csc(C2CCCN(C(=O)OC(C)(C)C)C2)n1)C(=O)NC(=C)C(=O)OC]

Using the procedure described for Compound 2, tert-butyl 3-(4-(((4S,7S)-4-(hydroxymethyl)-10,10,11,11-tetramethyl-3,6-dioxo-2,9-dioxa-5-aza-10-siladodecan-7-yl)carbamoyl)thiazol-2-yl)piperidine-1-carboxylate (0.728 g, 1.18 mmole) was converted to tert-butyl 3-(4-((3-((3-methoxy-3-oxoprop-1-en-2-yl)amino)-3-oxoprop-1-en-2-yl)carbamoyl)thiazol-2-yl)piperidine-1-carboxylate (0.126 g, 23%) as a white solid.

¹H NMR (400 MHz, Chloroform-d) δ 9.89 (s, 1H), 8.53 (s, 1H), 8.06 (s, 1H), 6.74 (d, J=2.2 Hz, 1H), 6.69 (s, 1H), 6.01 (d, J=1.2 Hz, 1H), 5.47 (t, J=1.9 Hz, 1H), 4.39-4.26 (m, 1H), 4.09-3.94 (m, 1H), 3.90 (s, 3H), 3.25-3.03 (m, 2H), 3.01-2.86 (m, 1H), 2.31-2.16 (m, 1H), 1.90-1.73 (m, 2H), 1.64 (s, 1H), 1.48 (s, 9H).

### Compound 4: Methyl 2-(2-(2-(4-(6-bromohexanamido)phenyl)thiazole-4-carboxamido)acrylamido)acrylate

### 6-Bromo-N-(4-(4,4,5,5-tetramethyl-1,3,2-dioxaborolan-2-yl)phenyl)hexanamide (4.1)

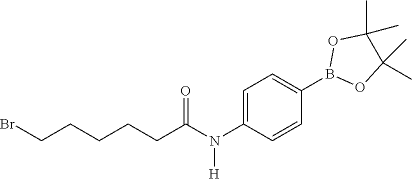 [SMILES: CC1(C)OB(c2ccc(NC(=O)CCCCCBr)cc2)OC1(C)C]

6-Bromohexanoic acid (1.964 g, 10.1 mmole) was dissolved in DCM (20 mL) and DMF (1 drop) was added. Oxalyl chloride (0.880 mL, 10.1 mmole) was added dropwise and the solution was stirred at RT for 90 minutes, then was concentrated. The residue was dissolved in DCM (2 mL) and added dropwise to cold (0° C.) solution of 4-(4,4,5,5-tetramethyl-1,3,2-dioxaborolan-2-yl)aniline (2.007 g, 9.16 mmole) and N,N-diisopropylethylamine (3.20 mL, 18.4 mmole) in DCM (20 mL). The resulting solution was stirred at 0° for 60 minutes then at RT for 2 hours. Water (50 mL) was added and the two layers were separated. The mixture was extracted with DCM (2×20 mL). The combined organics were dried over sodium sulfate, filtered, and concentrated. The crude residue was purified by silica gel chromatography (Isco CombiPrep, 24 g RediSep column, 10-30% ethyl acetate/hexane gradient) to provide 6-bromo-N-(4-(4,4,5,5-tetramethyl-1,3,2-dioxaborolan-2-yl)phenyl)hexanamide as an orange solid (3.241 g, 89%).

1H NMR (400 MHz, Chloroform-d) δ 7.81-7.72 (m, 2H), 7.52 (d, J=8.1 Hz, 2H), 7.17 (s, 1H), 3.42 (t, J=6.7 Hz, 2H), 2.38 (t, J=7.4 Hz, 2H), 1.96-1.85 (m, 2H), 1.79 (d, J=7.5 Hz, 2H), 1.57-1.48 (m, 2H), 1.34 (s, 12H).

### Methyl N-(2-(4-(6-bromohexanamido)phenyl)thiazole-4-carbonyl)-O-(tert-butyldimethylsilyl)-L-serinate (4.2)

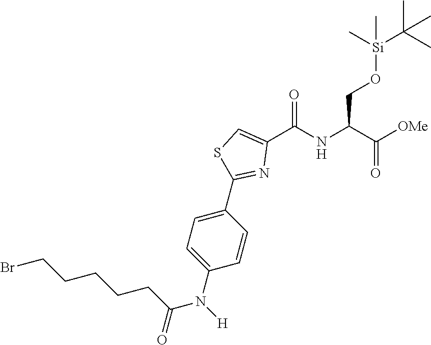 [SMILES: COC(=O)[C@H](CO[Si](C)(C)C(C)(C)C)NC(=O)c1csc(-c2ccc(NC(=O)CCCCCBr)cc2)n1]

Methyl N-(2-bromothiazole-4-carbonyl)-O-(tert-butyldimethylsilyl)-L-serinate (2.006 g, 4.74 mmole) was dissolved in 1,4-dioxane (9 mL). 6-Bromo-N-(4,4,5,5-tetramethyl-1,3,2-dioxaborolan-2-yl)phenyl)hexanamide (1.880 g, 4.75 mmole) was added followed by potassium carbonate (2M aqueous solution, 4.70 mL, 9.40 mmole) and bis(triphenylphosphine)palladium(II) chloride (0.332 g, 0.473 mmole). The mixture was heated to 85° C. for 18 hours and cooled to RT. Water (50 mL) was added and the mixture was extracted with ethyl acetate (3×20 mL). The combined organics were dried over magnesium sulfate, filtered, and concentrated. The crude residue was purified by silica gel chromatography (Isco CombiPrep, 24 g RediSep column, 20-50% ethyl acetate/hexane gradient) to provide methyl N-(2-(4-(6-bromohexanamido)phenyl)thiazole-4-carbonyl)-O-(tert-butyldimethylsilyl)-L-serinate as a thick, orange gel (1.290 g, 44%).

¹H NMR (400 MHz, Chloroform-d) δ 8.22 (d, J=8.7 Hz, 1H), 8.05 (s, 1H), 7.94-7.85 (m, 2H), 7.67-7.60 (m, 2H), 7.51 (s, 1H), 4.90-4.81 (m, 1H), 4.22 (dd, J=10.1, 2.6 Hz, 1H), 3.95 (dd, J=10.0, 3.4 Hz, 1H), 3.79 (s, 3H), 3.43 (t, J=6.7 Hz, 2H), 2.41 (t, J=7.4 Hz, 2H), 1.92 (dq, J=8.1, 6.8 Hz, 2H), 1.84-1.72 (m, 2H), 1.59-1.50 (m, 2H), 0.92 (s, 9H), 0.08 (s, 3H), 0.06 (s, 3H).

### N-(2-(4-(6-Bromohexanamido)phenyl)thiazole)-4-carbonyl)-O-(tert-butyldimethylsilyl)-L-serine (4.3)

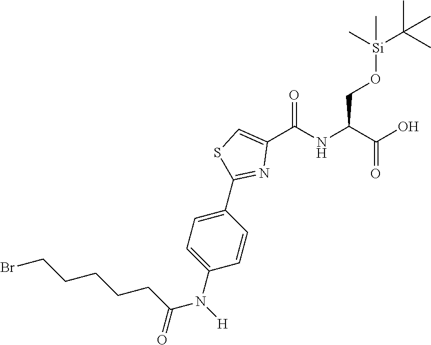 [SMILES: CC(C)(C)[Si](C)(C)OC[C@H](NC(=O)c1csc(-c2ccc(NC(=O)CCCCCBr)cc2)n1)C(=O)O]

Methyl N-(2-(4-(6-bromohexanamido)phenyl)thiazole-4-carbonyl)-O-(tert-butyldimethylsilyl)-L-serinate (1.290 g, 2.11 mmole) was dissolved in 4/1/1 THF/methanol/water (12 mL). Lithium hydroxide (0.158 g, 6.60 mmole) was added and the mixture was stirred at RT for 2 hours. The mixture was poured into water (40 mL) and the solution was treated with 1N aq. HCl to pH=4. The slurry was extracted with ethyl acetate (3×15 mL). The combined organics were dried over sodium sulfate, filtered, and concentrated to provide N-(2-(4-(6-bromohexanamido)phenyl)thiazole)-4-carbonyl)-O-(tert-butyldimethylsilyl)-L-serine as an orange foam (1.213 g, 96%).

¹H NMR (400 MHz, Chloroform-d) δ 8.26 (d, J=8.7 Hz, 1H), 8.05 (s, 1H), 7.84-7.81 (m, 2H), 7.74 (s, 1H), 7.67-7.60 (m, 2H), 4.90-4.81 (m, 1H), 4.26 (dd, J=10.1, 2.6 Hz, 1H), 3.95 (dd, J=10.0, 3.4 Hz, 1H), 3.43 (t, J=6.7 Hz, 2H), 2.41 (t, J=7.4 Hz, 2H), 1.92 (dq, J=8.1, 6.8 Hz, 2H), 1.84-1.72 (m, 2H), 1.55-1.48 (m, 2H), 0.92 (s, 9H), 0.08 (s, 3H), 0.06 (s, 3H).

### Methyl N-(2-(4-(6-bromohexanamido)phenyl)thiazole-4-carbonyl)-O-(tert-butyldimethylsilyl)-L-seryl-L-serinate (4.4)

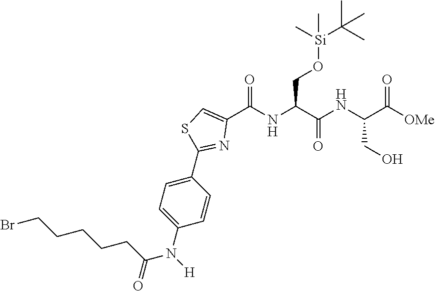 [SMILES: COC(=O)[C@H](CO)NC(=O)[C@H](CO[Si](C)(C)C(C)(C)C)NC(=O)c1csc(-c2ccc(NC(=O)CCCCCBr)cc2)n1]

Using the procedure described for Example 1.4, N-(2-(4-(6-bromohexanamido)phenyl)thiazole)-4-carbonyl)-O-(tert-butyldimethylsilyl)-L-serine (1.213 g, 2.03 mmole) was converted to methyl N-(2-(4-(6-bromohexanamido)phenyl)thiazole-4-carbonyl)-O-(tert-butyldimethylsilyl)-L-seryl-L-serinate (1.152 g, 81%) as a yellow gel.

¹H NMR (400 MHz, Chloroform-d) δ 8.24 (d, J=7.0 Hz, 1H), 8.06 (s, 1H), 7.90-7.84 (m, 2H), 7.65-7.60 (m, 2H), 7.49-7.45 (m, 1H), 7.42 (s, 1H), 4.69 (dq, J=7.6, 3.8 Hz, 1H), 4.64 (dt, J=6.7, 3.3 Hz, 1H), 4.25-4.16 (m, 1H), 4.05-3.93 (m, 2H), 3.89-3.81 (m, 1H), 3.79 (s, 3H), 3.44 (t, J=6.7 Hz, 2H), 2.43 (t, J=7.4 Hz, 2H), 1.84-1.73 (m, 3H), 1.57 (q, J=8.3 Hz, 3H), 0.95 (s, 9H), 0.14 (s, 3H), 0.14 (s, 3H).

### Methyl 2-(2-(2-(4-(6-bromohexanamido)phenyl)thiazole-4-carboxamido)acrylamido)acrylate (4)

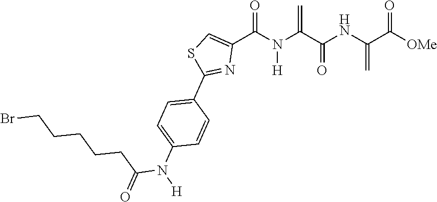 [SMILES: C=C(NC(=O)c1csc(-c2ccc(NC(=O)CCCCCBr)cc2)n1)C(=O)NC(=C)C(=O)OC]

Using the procedure described for Compound 2, methyl N-(2-(4-(6-bromohexanamido)phenyl)thiazole-4-carbonyl)-O-(tert-butyldimethylsilyl)-L-seryl-L-serinate (1.151 g, 1.64 mmole) was converted to methyl 2-(2-(2-(4-(6-bromohexanamido)phenyl)thiazole-4-carboxamido)acrylamide)acrylate (0.275 g, 31%) as a white solid.

¹H NMR (400 MHz, Chloroform-d) δ 10.00 (s, 1H), 8.55 (s, 1H), 8.11 (s, 1H), 8.04-7.90 (m, 2H), 7.64 (d, J=8.3 Hz, 2H), 6.78 (d, J=2.2 Hz, 1H), 6.74-6.65 (m, 1H), 6.03 (d, J=1.2 Hz, 1H), 5.50 (t, J=1.9 Hz, 1H), 3.91 (s, 3H), 3.44 (t, J=6.7 Hz, 2H), 2.43 (t, J=7.4 Hz, 2H), 1.93 (dq, J=9.1, 6.8 Hz, 2H), 1.80 (p, J=7.6 Hz, 2H), 1.63-1.50 (m, 2H).

### Compound 5: Methyl 2-(2-(2-(4-((tert-butoxycarbonyl)amino)phenyl)thiazole-4-carboxamido)acrylamido)acrylate

### Ethyl 2-(4-((tert-butoxycarbonyl)amino)phenyl)thiazole-4-carboxylate (5.1)

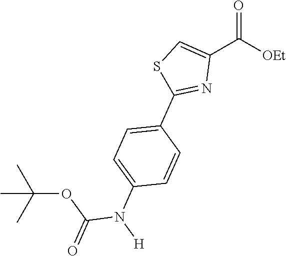 [SMILES: CCOC(=O)c1csc(-c2ccc(NC(=O)OC(C)(C)C)cc2)n1]

Ethyl 2-bromothiazole-4-carboxylate (1.006 g, 4.26 mmole) was dissolved in 1,4-dioxane (8.5 mL). Tert-butyl (4-(4,4,5,5-tetramethyl-1,3,2-dioxaborolan-2-yl)phenyl)carbamate (1.491 g, 4.67 mmole) was added followed by potassium carbonate (2M aqueous solution, 4.20 mL, 8.40 mmole) and bis(triphenylphosphine)palladium(II) chloride (0.149 g, 0.212 mmole). The mixture was heated to 90° C. for 20 hours and was cooled to RT. Water (50 mL) was added and the mixture was extracted with ethyl acetate (3×15 mL). The combined organics were dried over magnesium sulfate, filtered, and concentrated. The crude residue was purified by silica gel chromatography (Isco CombiPrep, 24 g RediSep column, 10-25% ethyl acetate/hexane gradient) to provide ethyl 2-(4-((tert-butoxycarbonyl)amino)phenyl)thiazole-4-carboxylate as a white solid (0.557 g, 38%).

¹H NMR (400 MHz, Chloroform-d) δ 8.10 (s, 1H), 7.99-7.90 (m, 2H), 7.50-7.41 (m, 2H), 6.62 (s, 1H), 4.44 (q, J=7.1 Hz, 2H), 1.53 (s, 9H), 1.43 (t, J=7.1 Hz, 3H).

### 2-(4-((Tert-butoxycarbonyl)amino)phenyl)thiazole-4-carboxylic Acid (5.2)

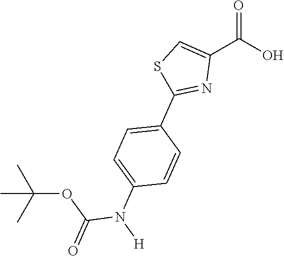 [SMILES: CC(C)(C)OC(=O)Nc1ccc(-c2nc(C(=O)O)cs2)cc1]

Using the procedure described for Example 1.3, ethyl 2-(4-((tert-butoxycarbonyl)amino)phenyl)thiazole-4-carboxylate (0.557 g, 1.60 mmole) was converted to 2-(4-((tert-butoxycarbonyl)amino)phenyl)thiazole-4-carboxylic acid (0.442 g, 86%) as a white solid.

¹H NMR (400 MHz, DMSO-d₆) δ 13.06 (s, 1H), 9.69 (s, 1H), 8.41 (s, 1H), 7.94-7.78 (m, 2H), 7.67-7.54 (m, 2H), 1.50 (s, 9H).

### 2,5-Dioxopyrrolidin-1-yl 2-(4-((tert-butoxycarbonyl)amino)phenyl)thiazole-4-carboxylate (5.3)

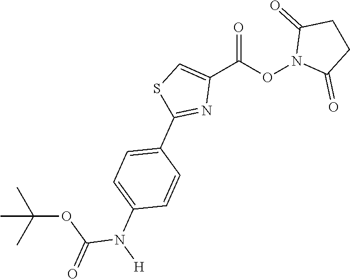 [SMILES: CC(C)(C)OC(=O)Nc1ccc(-c2nc(C(=O)ON3C(=O)CCC3=O)cs2)cc1]

2-(4-((Tert-butoxycarbonyl)amino)phenyl)thiazole-4-carboxylic acid (0.442 g, 1.38 mmole) was dissolved in DMF (5 mL). N-Hydroxysuccinimide (0.194 g, 1.69 mmole) was added followed by EDC·HCl (0.533 g, 2.78 mmole) and the mixture was stirred at RT for 90 minutes. Water (50 mL) was added and the mixture was extracted with ethyl acetate (3×15 mL). The combined organics were dried over magnesium sulfate, filtered, and concentrated. The crude residue was purified by silica gel chromatography (Isco CombiPrep, 12 g RediSep column, 30-50% ethyl acetate/hexane gradient) to provide 2,5-dioxopyrrolidin-1-yl 2-(4-((tert-butoxycarbonyl)amino)phenyl)thiazole-4-carboxylate as a white solid (0.376 g, 65%).

¹H NMR (400 MHz, Chloroform-d) δ 8.38 (s, 1H), 7.97-7.87 (m, 2H), 7.53-7.42 (m, 2H), 6.69 (s, 1H), 2.92 (s, 4H), 1.53 (s, 9H).

### Methyl (2-(4-((tert-butoxycarbonyl)amino)phenyl)-thiazole-4-carbonyl)-L-serinate (5.4)

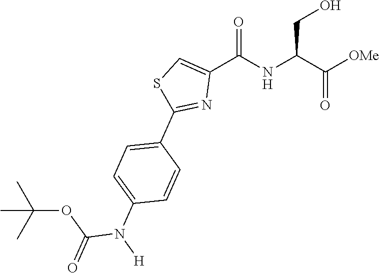 [SMILES: COC(=O)[C@H](CO)NC(=O)c1csc(-c2ccc(NC(=O)OC(C)(C)C)cc2)n1]

2,5-Dioxopyrrolidin-1-yl 2-(4-((tert-butoxycarbonyl)amino)phenyl)thiazole-4-carboxylate (0.376 g, 0.901 mmole) was suspended in DCM (4 mL). L-Serine methyl ester hydrochloride (0.168 g, 1.08 mmole) was added followed by N,N-diisopropylethylamine (0.320 mL, 1.84 mmole). The mixture was stirred at RT for 20 hours and water (25 mL) was added. The mixture was extracted with DCM (3×10 mL). The combined organics were dried over sodium sulfate, filtered, and concentrated. The crude residue was purified by silica gel chromatography (Isco CombiPrep, 12 g RediSep column, 40-70% ethyl acetate/hexane gradient) to provide methyl (2-(4-((tert-butoxycarbonyl)amino)phenyl)-thiazole-4-carbonyl)-L-serinate as a white solid (0.355 g, 93%).

¹H NMR (400 MHz, Chloroform-d) δ 8.24 (d, J=7.5 Hz, 1H), 8.06 (s, 1H), 7.94-7.85 (m, 2H), 7.51-7.43 (m, 2H), 6.64 (s, 1H), 4.88 (dt, J=7.6, 3.8 Hz, 1H), 4.17-4.05 (m, 2H), 3.85 (s, 3H), 2.63 (t, J=6.2 Hz, 1H), 1.54 (s, 9H).

### N-(2-(4-((Tert-butoxycarbonyl)amino)phenyl)thiazole-4-carbonyl)-O-(tert-butyldimethylsilyl)-L-serine (5.5)

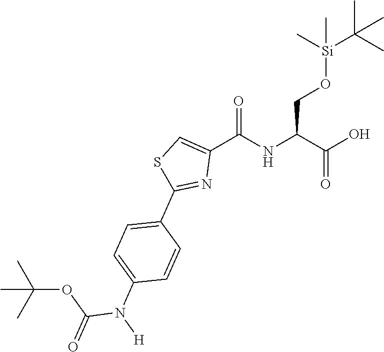 [SMILES: CC(C)(C)OC(=O)Nc1ccc(-c2nc(C(=O)N[C@@H](CO[Si](C)(C)C(C)(C)C)C(=O)O)cs2)cc1]

Using the general procedure described for Example 4.2 and 4.3, methyl (2-(4-((tert-butoxycarbonyl)amino)phenyl)-thiazole-4-carbonyl)-L-serinate (0.355 g, 0.842 mmole) was converted to N-(2-(4-((tert-butoxycarbonyl)amino)phenyl)thiazole-4-carbonyl)-O-(tert-butyldimethylsilyl)-L-serine (0.297 g, 68%) as a white solid.

¹H NMR (400 MHz, Chloroform-d) δ 8.25 (d, J=7.7 Hz, 1H), 8.08 (s, 1H), 7.94-7.86 (m, 2H), 7.45 (d, J=8.4 Hz, 2H), 4.84 (dt, J=8.0, 4.1 Hz, 1H), 4.27 (dd, J=10.1, 3.4 Hz, 1H), 3.98 (dd, J=10.0, 4.4 Hz, 1H), 1.56 (s, 9H), 0.92 (s, 9H), 0.08 (s, 6H).

### Methyl N-(2-(4-((tert-butoxycarbonyl)amino)phenyl)thiazole-4-carbonyl)-O-(tert-butyldimethylsilyl)-L-seryl-L-serinate (5.6)

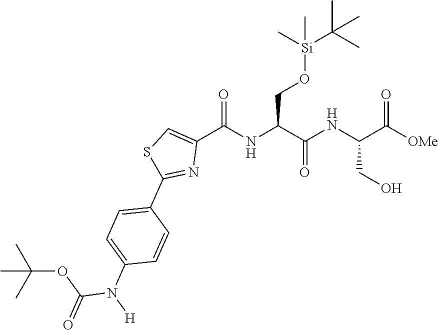 [SMILES: COC(=O)[C@H](CO)NC(=O)[C@H](CO[Si](C)(C)C(C)(C)C)NC(=O)c1csc(-c2ccc(NC(=O)OC(C)(C)C)cc2)n1]

Using the procedure described for Example 1.4, N-(2-(4-((tert-butoxycarbonyl)amino)phenyl)thiazole-4-carbonyl)-O-(tert-butyldimethylsilyl)-L-serine (0.297 g, 0.569 mmole) was converted to methyl N-(2-(4-((tert-butoxycarbonyl)amino)phenyl)thiazole-4-carbonyl)-O-(tert-butyldimethylsilyl)-L-seryl-L-serinate (0.385 g) as a colorless solid using pyBOP as the coupling reagent.

¹H NMR (400 MHz, Chloroform-d) δ 8.25 (d, J=7.0 Hz, 1H), 8.06 (d, J=4.9 Hz, 1H), 7.91-7.82 (m, 2H), 7.50-7.39 (m, 3H), 6.66 (s, 1H), 4.69 (dt, J=7.1, 3.6 Hz, 1H), 4.64 (dq, J=10.6, 4.0, 3.3 Hz, 1H), 4.21 (ddd, J=9.8, 3.9, 1.0 Hz, 1H), 3.99 (dd, J=3.7, 1.0 Hz, 2H), 3.84 (dd, J=9.8, 6.6 Hz, 1H), 3.78 (s, 3H), 1.54 (s, 9H), 0.94 (s, 9H), 0.14 (s, 3H), 0.13 (s, 3H).

### Methyl 2-(2-(2-(4-((tert-butoxycarbonyl)amino)phenyl)thiazole-4-carboxamido)acrylamido)acrylate (5)

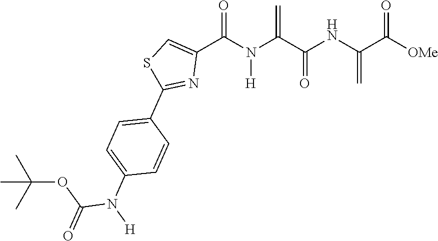 [SMILES: C=C(NC(=O)c1csc(-c2ccc(NC(=O)OC(C)(C)C)cc2)n1)C(=O)NC(=C)C(=O)OC]

Using the procedure describing Compound 5, methyl N-(2-(4-((tert-butoxycarbonyl)amino)phenyl)thiazole-4-carbonyl)-O-(tert-butyldimethylsilyl)-L-seryl-L-serinate (0.385 g, 0.618 mmole) was converted to methyl 2-(2-(2-(4-((tert-butoxycarbonyl)amino)phenyl)thiazole-4-carboxamido)acrylamido)acrylate (0.033 g, 12%).

¹H NMR (400 MHz, DMSO-d₆) δ 9.97 (s, 1H), 9.93 (s, 1H), 9.72 (s, 1H), 8.40 (s, 1H), 7.96-7.84 (m, 2H), 7.69-7.58 (m, 2H), 6.51 (d, J=1.3 Hz, 1H), 5.87 (s, 2H), 5.84 (s, 1H), 3.75 (s, 3H), 1.49 (s, 9H).

### Compound 6: Methyl 2-(2-(2-(6-((6-((methylsulfonyl)oxy)hexyl)carbamoyl)pyridine-3-yl)thiazole-4-carboxamido)acrylamido)acrylate

### 5-Bromo-N-(6-hydroxyhexyl)picolinamide (6.1)

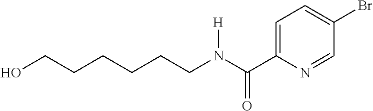 [SMILES: O=C(NCCCCCCO)c1ccc(Br)cn1]

5-Bromopicolinic acid (1.006 g, 4.98 mmole) was suspended in DCM (10 mL) and N,N′-carbonyldiimidazole (0.887 g, 5.47 mmole) was added (gas evolution was observed). The mixture was stirred at RT for 90 minutes and 6-aminohexan-1-ol (0.706 g, 6.02 mmole) was added. The resulting solution was stirred at RT for 3 hours then water (50 mL) was added. The two layers were separated and the aqueous layer was extracted with DCM (2×15 mL). The combined organics were dried over sodium sulfate, filtered, and concentrated. The crude residue was purified by silica gel chromatography to provide 5-bromo-N-(6-hydroxyhexyl)picolinamide (1.019 g, 68%) as a white solid.

¹H NMR (400 MHz, Chloroform-d) δ 8.60 (dd, J=2.2, 0.8 Hz, 1H), 8.09 (dd, J=8.3, 0.7 Hz, 1H), 7.98 (dd, J=8.4, 2.3 Hz, 1H), 7.93 (s, 1H), 3.64 (t, J=6.5 Hz, 2H), 3.47 (td, J=7.1, 6.2 Hz, 2H), 1.76-1.55 (m, 4H), 1.43 (p, J=3.6 Hz, 4H).

### 5-Bromo-N-(6-((tert-butyldimethylsilyl)oxy)hexyl)picolinamide (6.2)

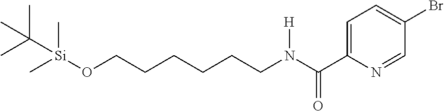 [SMILES: CC(C)(C)[Si](C)(C)OCCCCCCNC(=O)c1ccc(Br)cn1]

5-Bromo-N-(6-hydroxyhexyl)picolinamide (1.019 g, 3.38 mmole) was dissolved in DMF (7 mL). Imidazole (0.258 g, 3.79 mmole) was added followed by tert-butyldimethylchlorosilane (0.571 g, 3.79 mmole). The solution was stirred at RT for 2 hours and was poured into water (50 mL). The mixture was extracted with ethyl acetate (3×15 mL). The combined organics were dried over magnesium sulfate, filtered, and concentrated. The crude residue was purified by silica gel chromatography to provide 5-bromo-N-(6-((tert-butyldimethylsilyl)oxy)hexyl)picolinamide as a colorless oil (1.260 g, 90%).

¹H NMR (400 MHz, Chloroform-d) δ 8.59 (dd, J=2.3, 0.8 Hz, 1H), 8.09 (dd, J=8.3, 0.7 Hz, 1H), 7.97 (dd, J=8.3, 2.3 Hz, 1H), 7.91 (s, 1H), 3.60 (t, J=6.5 Hz, 2H), 3.45 (td, J=7.2, 6.1 Hz, 2H), 1.64 (dd, J=8.5, 6.0 Hz, 2H), 1.53 (p, J=6.6 Hz, 2H), 1.45-1.34 (m, 4H), 0.89 (s, 9H), 0.04 (s, 6H).

### N-(6-((Tert-butyldimethylsilyl)oxy)hexyl)-5-(4,4,5,5-tetramethyl-1,3,2-dioxaborolan-2-yl)picolinamide (6.3)

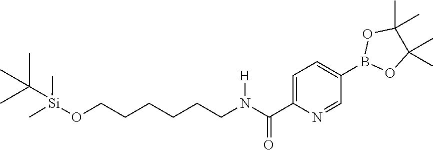 [SMILES: CC1(C)OB(c2ccc(C(=O)NCCCCCCO[Si](C)(C)C(C)(C)C)nc2)OC1(C)C]

5-Bromo-N-(6-((tert-butyldimethylsilyl)oxy)hexyl)picolinamide (1.260 g, 3.03 mmole) was dissolved in 1,4-dioxane (18 mL) and placed under an argon atmosphere. Bis(pinacolato)diboron (1.165 g, 4.59 mmole) was added followed by potassium acetate (0.745 g, 7.59 mmole) and 1,1-bis(diphenylphosphino)ferrocenepalladium(II) dichloride (0.221 g, 0.302 mmole). The mixture was heated to 85° C. for 18 hours and was cooled to RT. Water (75 mL) was added and the mixture was extracted with ethyl acetate (3×25 mL). The combined organics were dried over magnesium sulfate, filtered, and concentrated. The crude residue was purified by silica gel chromatography to provide N-(6-((tert-butyldimethylsilyl)oxy)hexyl)-5-(4,4,5,5-tetramethyl-1,3,2-dioxaborolan-2-yl)picolinamide as a brown oil (1.952 g).

¹H NMR (400 MHz, Chloroform-d) δ 8.85 (dd, J=1.6, 1.0 Hz, 1H), 8.21 (dd, J=7.7, 1.7 Hz, 1H), 8.17 (dd, J=7.8, 1.0 Hz, 1H), 8.12 (d, J=6.2 Hz, 1H), 3.60 (t, J=6.5 Hz, 2H), 3.47 (td, J=7.2, 6.1 Hz, 2H), 1.65 (h, J=6.8 Hz, 2H), 1.53 (p, J=6.6 Hz, 2H), 1.36 (s, 16H), 0.89 (s, 9H), 0.04 (s, 6H).

### Methyl O-(tert-butyldimethylsilyl)-N-(2-(6-((5-(tert-butyldimethylsilyl)oxy)pentyl)carbamoyl)pyridine-3-yl)thiazole-4-carbonyl)-L-serinate (6.4)

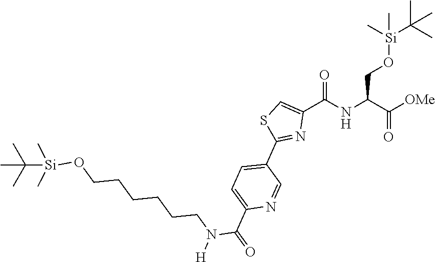 [SMILES: COC(=O)[C@H](CO[Si](C)(C)C(C)(C)C)NC(=O)c1csc(-c2ccc(C(=O)NCCCCCCO[Si](C)(C)C(C)(C)C)nc2)n1]

Methyl N-(2-bromothiazole-4-carbonyl)-O-(tert-butyldimethylsilyl)-L-serinate (0.759 g, 1.79 mmole) was dissolved in 1,4-dioxane (3.6 mL) and N-(6-((tert-butyldimethylsilyl)oxy)hexyl)-5-(4,4,5,5-tetramethyl-1,3,2-dioxaborolan-2-yl)picolinamide (1.073 g, 2.32 mmole) was added. Potassium carbonate (2M aqueous solution, 1.80 mL, 3.60 mmole) and bis(triphenylphosphine)palladium(II) chloride (0.132 g, 0.188 mmole) were added and the mixture was heated to 85° C. for 3 hours. The mixture was cooled to RT and poured into water (50 mL) and was extracted with ethyl acetate (3×15 mL). The combined organics were dried over magnesium sulfate, filtered, and concentrated. The crude residue was purified by silica gel chromatography to provide methyl O-(tert-butyldimethylsilyl)-N-(2-(6-((5-(tert-butyldimethylsilyl)oxy)pentyl)carbamoyl)pyridine-3-yl)thiazole-4-carbonyl)-L-serinate as an orange oil (0.733 g, 62%).

¹H NMR (400 MHz, Chloroform-d) δ 9.10 (dd, J=2.2, 0.9 Hz, 1H), 8.40 (dd, J=8.2, 2.2 Hz, 1H), 8.30 (dd, J=8.2, 0.9 Hz, 1H), 8.23 (s, 1H), 8.19 (d, J=8.8 Hz, 1H), 8.04 (t, J=6.0 Hz, 1H), 4.87 (dt, J=8.7, 3.0 Hz, 1H), 4.22 (dd, J=10.1, 2.6 Hz, 1H), 3.96 (dd, J=10.1, 3.4 Hz, 1H), 3.80 (s, 3H), 3.61 (t, J=6.5 Hz, 2H), 3.50 (td, J=7.2, 6.1 Hz, 2H), 1.68 (p, J=7.4 Hz, 2H), 1.54 (q, J=6.7 Hz, 2H), 1.42 (dt, J=8.7, 5.5 Hz, 4H), 0.92 (s, 9H), 0.89 (s, 9H), 0.08 (s, 3H), 0.06 (s, 3H), 0.05 (s, 6H).

### O-(Tert-butyldimethylsilyl)-N-(2-(6-((5-((tert-butyldimethylsilyl)oxy)pentyl)carbamoyl)pyridine-3-yl)thiazole-4-carbonyl)-L-serine (6.5)

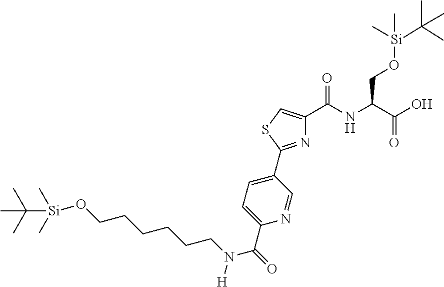 [SMILES: CC(C)(C)[Si](C)(C)OCCCCCCNC(=O)c1ccc(-c2nc(C(=O)N[C@@H](CO[Si](C)(C)C(C)(C)C)C(=O)O)cs2)cn1]

Methyl O-(tert-butyldimethylsilyl)-N-(2-(6-((5-(tert-butyldimethylsilyl)oxy)pentyl)carbamoyl)pyridine-3-yl)thiazole-4-carbonyl)-L-serinate (0.733 g, 1.10 mmole) was dissolved in 4/1/1 THF/methanol/water (6 mL). Lithium hydroxide (0.080 g, 3.34 mmole) was added and the mixture was stirred at RT for 3 hours. The mixture was poured into water (25 mL) and treated with 1N aq. HCl to pH=4. The slurry was extracted with ethyl acetate (3×10 mL). The combined organics were dried over sodium sulfate, filtered, and concentrated to provide O-(tert-butyldimethylsilyl)-N-(2-(6-((5-((tert-butyldimethylsilyl)oxy)pentyl)carbamoyl)pyridine-3-yl)thiazole-4-carbonyl)-L-serine as an orange solid (0.677 g, 95%).

1H NMR (400 MHz, Chloroform-d) δ 9.08 (dd, J=2.2, 0.8 Hz, 1H), 8.47-8.40 (m, 1H), 8.36 (dt, J=8.2, 1.1 Hz, 1H), 8.28 (d, J=8.8 Hz, 1H), 8.23 (d, J=8.1 Hz, 1H), 8.09 (t, J=6.1 Hz, 1H), 4.88 (ddd, J=8.1, 4.1, 3.0 Hz, 1H), 4.33-4.24 (m, 1H), 3.99 (dd, J=10.2, 4.0 Hz, 1H), 3.62 (t, J=6.4 Hz, 2H), 3.51 (q, J=7.0 Hz, 2H), 1.67 (q, J=7.6, 7.2 Hz, 2H), 1.59-1.50 (m, 2H), 1.42 (tq, J=9.5, 5.3, 4.2 Hz, 4H), 0.94-0.91 (m, 9H), 0.89 (s, 9H), 0.09 (s, 6H), 0.05 (s, 6H).

### Methyl O-(tert-butyldimethylsilyl)-N-(2-(6-((5-((tert-butyldimethylsilyl)oxy)pentyl)carbamoyl)pyridine-3-yl)thiazole-4-carbonyl)-L-seryl-L-serinate (6.6)

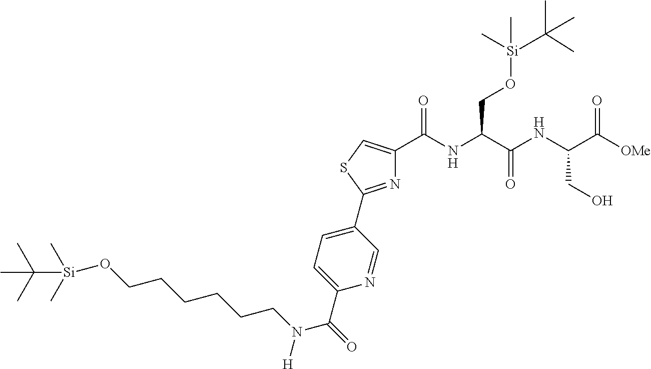 [SMILES: COC(=O)[C@H](CO)NC(=O)[C@H](CO[Si](C)(C)C(C)(C)C)NC(=O)c1csc(-c2ccc(C(=O)NCCCCCCO[Si](C)(C)C(C)(C)C)nc2)n1]

Using the procedure described for Example 1.4, O-(tert-butyldimethylsilyl)-N-(2-(6-((5-((tert-butyldimethylsilyl)oxy)pentyl)carbamoyl)pyridine-3-yl)thiazole-4-carbonyl)-L-serine (0.677 g, 1.04 mmole) was converted to methyl O-(tert-butyldimethylsilyl)-N-(2-(6-((5-((tert-butyldimethylsilyl)oxy)pentyl)carbamoyl)pyridine-3-yl)thiazole-4-carbonyl)-L-seryl-L-serinate (0.534 g, 68%) as a pale yellow oil using pyBOP as the coupling reagent.

1H NMR (400 MHz, Chloroform-d) δ 9.09 (ddd, J=4.3, 2.2, 0.9 Hz, 1H), 8.39 (ddd, J=8.2, 4.8, 2.2 Hz, 1H), 8.33-8.26 (m, 1H), 8.24 (d, J=3.8 Hz, 2H), 8.04 (t, J=6.0 Hz, 1H), 7.47 (d, J=7.1 Hz, 1H), 4.75-4.61 (m, 2H), 4.22 (dt, J=9.8, 3.8 Hz, 1H), 4.04-3.96 (m, 2H), 3.86 (dt, J=9.9, 6.5 Hz, 1H), 3.79 (s, 3H), 3.61 (t, J=6.5 Hz, 2H), 3.50 (q, J=6.8 Hz, 2H), 1.69 (q, J=7.2 Hz, 2H), 1.54 (p, J=6.7 Hz, 2H), 1.41 (dd, J=7.4, 4.3 Hz, 4H), 0.95 (s, 9H), 0.89 (s, 9H), 0.16 (s, 3H), 0.14 (s, 3H), 0.05 (s, 6H).

### Methyl 2-(2-(2-(6-((6-((methylsulfonyl)oxy)hexyl)carbamoyl)pyridine-3-yl)thiazole-4-carboxamido)acrylamido)acrylate (6)

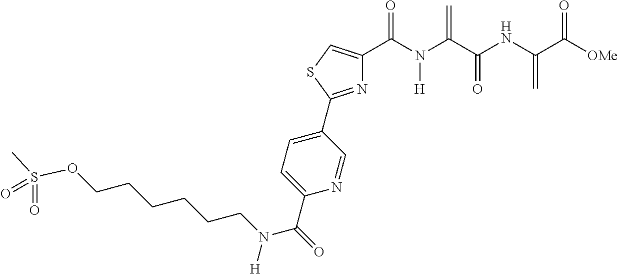 [SMILES: C=C(NC(=O)c1csc(-c2ccc(C(=O)NCCCCCCOS(C)(=O)=O)nc2)n1)C(=O)NC(=C)C(=O)OC]

Using the procedure described for Compound 2, methyl O-(tert-butyldimethylsilyl)-N-(2-(6-((5-((tert-butyldimethylsilyl)oxy)pentyl)carbamoyl)pyridine-3-yl)thiazole-4-carbonyl)-L-seryl-L-serinate (0.534 g, 0.710 mmole) was converted to methyl 2-(2-(2-(6-((6-((methylsulfonyl)oxy)hexyl)carbamoyl)pyridine-3-yl)thiazole-4-carboxamido)acrylamide)acrylate (0.073 g, 18%) as a white solid.

¹H NMR (400 MHz, Chloroform-d) δ 10.02 (s, 1H), 9.19 (dd, J=2.2, 0.8 Hz, 1H), 8.57 (s, 1H), 8.44 (dd, J=8.1, 2.2 Hz, 1H), 8.31 (dd, J=8.1, 0.8 Hz, 1H), 8.27 (s, 1H), 8.06 (t, J=6.1 Hz, 1H), 6.80 (d, J=2.3 Hz, 1H), 6.75-6.66 (m, 1H), 6.09-5.98 (m, 1H), 5.53 (t, J=2.0 Hz, 1H), 4.24 (t, J=6.5 Hz, 2H), 3.91 (s, 3H), 3.52 (q, J=6.8 Hz, 2H), 3.01 (s, 3H), 1.79 (t, J=7.0 Hz, 2H), 1.69 (p, J=7.1 Hz, 2H), 1.52-1.43 (m, 4H).

### Compound 7: Methyl 2-(2-(2-(4-methoxyphenyl)thiazole-4-carboxamido)acrylamido)acrylate

### Ethyl 2-(4-methoxyphenyl)thiazole-4-carboxylate (7.1)

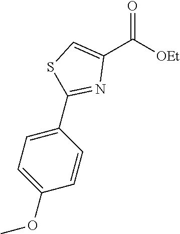 [SMILES: CCOC(=O)c1csc(-c2ccc(OC)cc2)n1]

Ethyl 2-bromothiazole-4-carboxylate (0.500 g, 2.12 mmole) was dissolved in 1,4-dioxane (4 mL). 2-(4-Methoxyphenyl)-4,4,5,5-tetramethyl-1,3,2-dioxaborolane (0.593 g, 2.53 mmole) was added followed by potassium carbonate (2M aqueous solution, 2.10 mL, 4.20 mmole) and bis(triphenylphosphine)palladium(II) chloride (0.147 g, 0.209 mmole). The mixture was heated to 80° C. for 5 hours and was cooled to RT. Water (25 mL) was added and the mixture was extracted with ethyl acetate (3×10 mL). The combined organics were dried over magnesium sulfate, filtered, and concentrated. The crude residue was purified by silica gel chromatography (Isco CombiPrep, 12 g Silicycle column, 10-30% ethyl acetate/hexane gradient) to provide ethyl 2-(4-methoxyphenyl)thiazole-4-carboxylate as a white solid (0.249 g, 45%).

¹H NMR (400 MHz, Chloroform-d) δ 8.09 (s, 1H), 7.99-7.90 (m, 2H), 7.00-6.90 (m, 2H), 4.44 (q, J=7.1 Hz, 2H), 3.87 (s, 3H), 1.43 (t, J=7.1 Hz, 3H).

### 2-(4-Methoxyphenyl)thiazole-4-carboxylic Acid (7.2)

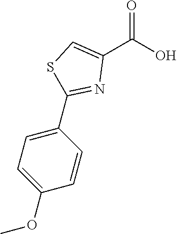 [SMILES: COc1ccc(-c2nc(C(=O)O)cs2)cc1]

Using the procedure described for Example 4.3, ethyl 2-(4-methoxyphenyl)thiazole-4-carboxylate (0.249 g, 0.946 mmole) was converted to 2-(4-methoxyphenyl)thiazole-4-carboxylic acid (0.248 g, 100%) as a cream colored solid.

¹H NMR (400 MHz, DMSO-d₆) δ 13.05 (s, 1H), 8.41 (s, 1H), 7.98-7.85 (m, 2H), 7.15-7.01 (m, 2H), 3.84 (s, 3H).

### Methyl (2-(4-methoxyphenyl)thiazole-4-carbonyl)-L-serinate (7.3)

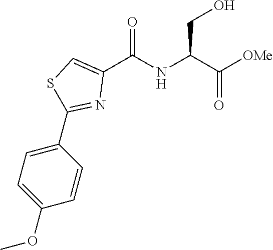 [SMILES: COC(=O)[C@H](CO)NC(=O)c1csc(-c2ccc(OC)cc2)n1]

Using the procedure described for Example 1.4, 2-(4-methoxyphenyl)thiazole-4-carboxylic acid (0.248 g, 1.05 mmole) was converted to methyl (2-(4-methoxyphenyl)thiazole-4-carbonyl)-L-serinate (0.292 g, 83%) as a white solid.

¹H NMR (400 MHz, Chloroform-d) δ 8.26 (d, J=7.5 Hz, 1H), 8.05 (s, 1H), 7.96-7.86 (m, 2H), 7.02-6.92 (m, 2H), 4.88 (dt, J=7.6, 3.8 Hz, 1H), 4.17-4.05 (m, 2H), 3.88 (s, 3H), 3.85 (s, 3H).

### O-(Tert-butyldimethylsilyl)-N-(2-(4-methoxyphenyl)thiazole-4-carbonyl)-L-serine (7.4)

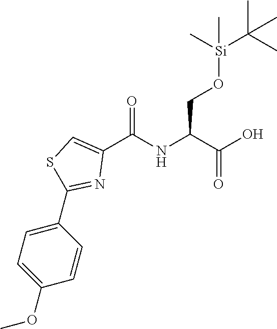 [SMILES: COc1ccc(-c2nc(C(=O)N[C@@H](CO[Si](C)(C)C(C)(C)C)C(=O)O)cs2)cc1]

Using the procedure described for Example 4.2 and 4.3, methyl (2-(4-methoxyphenyl)thiazole-4-carbonyl)-L-serinate (0.292 g, 0.868 mmole) was converted to O-(tert-butyldimethylsilyl)-N-(2-(4-methoxyphenyl)thiazole-4-carbonyl)-L-serine (0.269 g) as a mixture of products that was carried on without additional purification.

### Methyl O-(tert-butyldimethylsilyl)-N-(2-(4-methoxyphenyl)thiazole-4-carbonyl)-L-seryl-L-serinate (7.5)

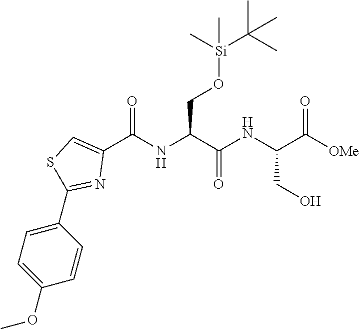 [SMILES: COC(=O)[C@H](CO)NC(=O)[C@H](CO[Si](C)(C)C(C)(C)C)NC(=O)c1csc(-c2ccc(OC)cc2)n1]

Using the procedure described for Example 1.4, O-(tert-butyldimethylsilyl)-N-(2-(4-methoxyphenyl)thiazole-4-carbonyl)-L-serine (0.269 g) was converted to methyl O-(tert-butyldimethylsilyl)-N-(2-(4-methoxyphenyl)thiazole-4-carbonyl)-L-seryl-L-serinate (0.097 g, 29%) as a white solid.

¹H NMR (400 MHz, CDCl₃) δ 8.26 (d, J=7.0 Hz, 1H), 8.03 (s, 1H), 7.93-7.83 (m, 2H), 7.46 (d, J=7.1 Hz, 1H), 6.99-6.90 (m, 2H), 4.70 (dd, J=7.2, 3.7 Hz, 1H), 4.65 (td, J=6.9, 4.2 Hz, 1H), 4.21 (dd, J=9.8, 4.1 Hz, 1H), 3.99 (dd, J=3.7, 1.0 Hz, 2H), 3.88 (s, 3H), 3.84 (dd, J=9.8, 6.7 Hz, 1H), 3.79 (s, 3H), 0.95 (s, 9H), 0.14 (s, 3H), 0.13 (s, 3H).

### Methyl 2-(2-(2-(4-methoxyphenyl)thiazole-4-carboxamido)acrylamido)acrylate (7)

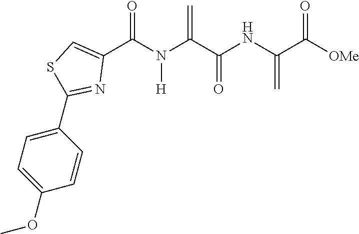 [SMILES: C=C(NC(=O)c1csc(-c2ccc(OC)cc2)n1)C(=O)NC(=C)C(=O)OC]

Using the procedure described in Compound 2, methyl O-(tert-butyldimethylsilyl)-N-(2-(4-methoxyphenyl)thiazole-4-carbonyl)-L-seryl-L-serinate (0.097 g, 0.180 mmole) was converted to methyl 2-(2-(2-(4-methoxyphenyl)thiazole-4-carboxamido)acrylamido)acrylate (0.0203 g, 29%) as a white solid.

¹H NMR (400 MHz, CDCl₃) δ 10.02 (s, 1H), 8.55 (s, 1H), 8.07 (s, 1H), 8.00-7.92 (m, 2H), 7.02-6.92 (m, 2H), 6.78 (d, J=2.2 Hz, 1H), 6.71 (s, 1H), 6.03 (d, J=1.3 Hz, 1H), 5.49 (t, J=1.9 Hz, 1H), 3.90 (s, 3H), 3.88 (s, 3H).

### Compound 8: Methyl 2-(2-(2-(4-(((tert-butoxycarbonyl)amino)methyl)phenyl)thiazole-4-carboxamido)acrylamido)acrylate

### Tert-butyl (4-bromobenzyl)carbamate (8.1)

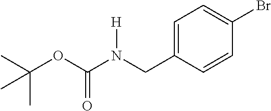 [SMILES: CC(C)(C)OC(=O)NCc1ccc(Br)cc1]

4-Bromobenzylamine (2.006 g, 10.8 mmole) was dissolved in DCM (22 mL) and N,N-diisopropylethylamine (2.45 mL, 14.1 mmole) was added. Di-tert-butyl dicarbonate (2.600 g, 11.9 mmole) was added and the solution was stirred at RT for 5 hours. The mixture was concentrated and the crude residue was purified by silica gel chromatography (Isco CombiPrep, 40 g Silicycle column, 10-25% ethyl acetate/hexane gradient) to provide tert-butyl (4-bromobenzyl)carbamate as a white solid (2.732 g, 88%).

¹H NMR (400 MHz, Chloroform-d) δ 7.48-7.39 (m, 2H), 7.20-7.11 (m, 2H), 4.84 (s, 1H), 4.26 (d, J=6.1 Hz, 2H), 1.46 (s, 9H).

### Tert-butyl (4-(4,4,5,5-tetramethyl-1,3,2-dioxaborolan-2-yl)benzyl)carbamate (8.2)

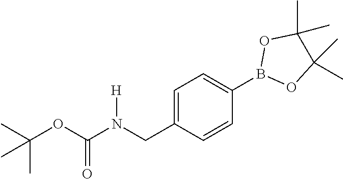 [SMILES: CC(C)(C)OC(=O)NCc1ccc(B2OC(C)(C)C(C)(C)O2)cc1]

Tert-butyl (4-bromobenzyl)carbamate (2.732 g, 9.55 mmole) was dissolved in 1,4-dioxane (19 mL). Bis(pinacolato)diboron (2.914 g, 11.5 mmole) was added followed by potassium acetate (2.032 g, 20.7 mmole) and 1,1′-bis(diphenylphosphino)ferrocenepalladium(II) dichloride (0.697 g, 0.953 mmole). The mixture was heated to 80° C. for 5 hours and was cooled to RT. Water (50 mL) was added and the mixture was extracted with ethyl acetate (3×20 mL). The combined organics were dried over magnesium sulfate, filtered, and concentrated. The crude residue was purified by silica gel chromatography (Isco CombiPrep, 40 g Silicycle column, 5-20% ethyl acetate/hexane gradient) to provide tert-butyl (4-(4,4,5,5-tetramethyl-1,3,2-dioxaborolan-2-yl)benzyl)carbamate as a golden oil (4.231 g) that was carried on without additional purification. ¹H NMR (400 MHz, Chloroform-d) δ 7.80-7.73 (m, 2H), 7.28 (d, J=8.1 Hz, 2H), 4.89 (s, 1H), 4.32 (d, J=6.0 Hz, 2H), 1.45 (s, 9H), 1.34 (s, 12H).

### Ethyl 2-(4-(((tert-butoxycarbonyl)amino)methyl)phenyl)thiazole-4-carboxylate (8.3)

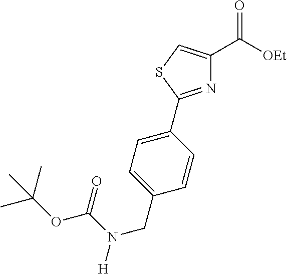 [SMILES: CCOC(=O)c1csc(-c2ccc(CNC(=O)OC(C)(C)C)cc2)n1]

Ethyl 2-bromothiazole-4-carboxylate (0.504 g, 2.13 mmole) was dissolved in 1,4-dioxane (4 mL). Tert-butyl (4-(4,4,5,5-tetramethyl-1,3,2-dioxaborolan-2-yl)benzyl)carbamate (0.862 g, 2.59 mmole) was added followed by potassium carbonate (2M aq. solution, 2.10 mL, 4.20 mmole) and bis(triphenylphosphine)palladium(II) chloride (0.149 g, 0.212 mmole). The mixture was heated to 80° C. for 20 hours. The mixture was cooled to RT and water (50 mL) was added. The mixture was extracted with ethyl acetate (3×15 mL). The combined organics were dried over magnesium sulfate, filtered, and concentrated. The crude residue was purified by silica gel chromatography (Isco CombiPrep, 12 g Silicycle column, 10-30% ethyl acetate/hexane gradient) to provide ethyl 2-(4-(((tert-butoxycarbonyl)amino)methyl)phenyl)thiazole-4-carboxylate as a yellow solid (0.400 g, 52%).

¹H NMR (400 MHz, Chloroform-d) δ 8.15 (s, 1H), 8.01-7.92 (m, 2H), 7.41-7.32 (m, 2H), 4.92 (s, 1H), 4.51-4.40 (m, 2H), 4.35 (dd, J=9.9, 6.7 Hz, 2H), 1.51-1.45 (m, 9H), 1.45-1.40 (m, 3H).

### 2-(4-(((Tert-butoxycarbonyl)amino)methyl)phenyl)thiazole-4-carboxylic Acid (8.4)

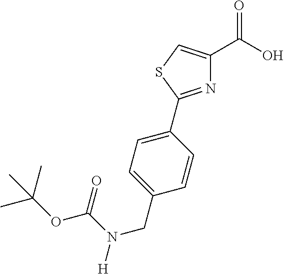 [SMILES: CC(C)(C)OC(=O)NCc1ccc(-c2nc(C(=O)O)cs2)cc1]

Using the procedure described for Example 4.3, ethyl 2-(4-(((tert-butoxycarbonyl)amino)methyl)phenyl)thiazole-4-carboxylate (0.400 g, 1.10 mmole) was converted to 2-(4-(((tert-butoxycarbonyl)amino)methyl)phenyl)thiazole-4-carboxylic acid (0.338 g, 92%) as a white solid.

¹H NMR (400 MHz, DMSO-d₆) δ 13.10 (s, 1H), 8.48 (s, 1H), 8.00-7.84 (m, 2H), 7.48 (t, J=6.2 Hz, 1H), 7.38 (d, J=8.2 Hz, 2H), 4.19 (d, J=6.2 Hz, 2H), 1.40 (s, 9H).

### Methyl (2-(4-(((tert-butoxycarbonyl)amino)methyl)phenyl)thiazole-4-carbonyl)-L-serinate (8.5)

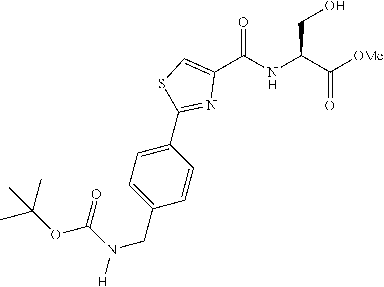 [SMILES: COC(=O)[C@H](CO)NC(=O)c1csc(-c2ccc(CNC(=O)OC(C)(C)C)cc2)n1]

Using the procedure described for Example 1.4, 2-(4-(((tert-butoxycarbonyl)amino)methyl)phenyl)thiazole-4-carboxylic acid (0.338 g, 1.01 mmole) was converted to methyl (2-(4-(((tert-butoxycarbonyl)amino)methyl)phenyl)thiazole-4-carbonyl)-L-serinate (0.336 g, 76%) as a white solid.

¹H NMR (400 MHz, Chloroform-d) δ 8.26 (d, J=7.5 Hz, 1H), 8.11 (s, 1H), 7.92 (d, J=8.1 Hz, 2H), 7.37 (d, J=8.0 Hz, 2H), 4.95 (s, 1H), 4.88 (dt, J=7.5, 3.8 Hz, 1H), 4.37 (d, J=6.1 Hz, 2H), 4.17-4.05 (m, 2H), 3.85 (s, 3H), 1.48 (s, 9H).

### N-(2-(4-(((Tert-butoxycarbonyl)amino)methyl)-phenyl)thiazole-4-carbonyl)-O-(tert-butyldimethylsilyl)-L-serine (8.6)

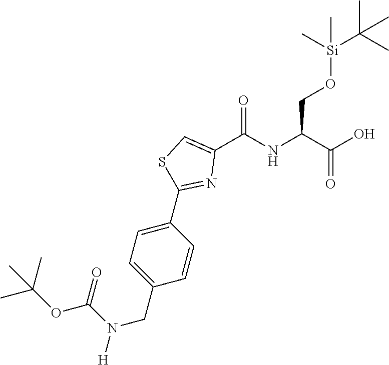 [SMILES: CC(C)(C)OC(=O)NCc1ccc(-c2nc(C(=O)N[C@@H](CO[Si](C)(C)C(C)(C)C)C(=O)O)cs2)cc1]

Using the procedure described for Example 4.2 and 4.3, methyl (2-(4-(((tert-butoxycarbonyl)amino)methyl)phenyl)thiazole-4-carbonyl)-L-serinate (0.336 g, 0.772 mmole) was converted to N-(2-(4-(((tert-butoxycarbonyl)amino)methyl)-phenyl)thiazole-4-carbonyl)-O-(tert-butyldimethylsilyl)-L-serine (0.334 g, 81%) as a white solid.

¹H NMR (400 MHz, DMSO-d₆) δ 8.41 (s, 1H), 8.23 (d, J=8.6 Hz, 1H), 8.04-7.94 (m, 2H), 7.53 (t, J=6.2 Hz, 1H), 7.43 (d, J=8.0 Hz, 2H), 4.63 (dt, J=8.6, 3.5 Hz, 1H), 4.24 (d, J=6.2 Hz, 2H), 4.16 (dd, J=10.3, 3.4 Hz, 1H), 3.99 (dd, J=10.3, 3.7 Hz, 1H), 1.45 (s, 9H), 0.93 (s, 9H), 0.10 (s, 3H), 0.08 (s, 3H).

### Methyl N-(2-(4-(((tert-butoxycarbonyl)amino)methyl)-phenyl)thiazole-4-carbonyl)-O-(tert-butyldimethylsilyl)-L-seryl-L-serinate (8.7)

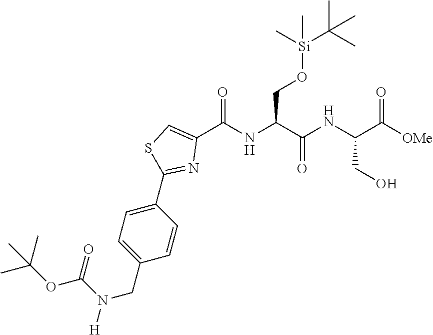 [SMILES: COC(=O)[C@H](CO)NC(=O)[C@H](CO[Si](C)(C)C(C)(C)C)NC(=O)c1csc(-c2ccc(CNC(=O)OC(C)(C)C)cc2)n1]

Using the procedure described for Example 1.4, N-(2-(4-(((tert-butoxycarbonyl)amino)methyl)-phenyl)thiazole-4-carbonyl)-O-(tert-butyldimethylsilyl)-L-serine (0.334 g, 0.623 mmole) was converted to methyl N-(2-(4-(((tert-butoxycarbonyl)amino)methyl)-phenyl)thiazole-4-carbonyl)-O-(tert-butyldimethylsilyl)-L-seryl-L-serinate (0.263 g, 66%) as a colorless gel.

¹H NMR (400 MHz, CDCl₃) δ 8.26 (d, J=7.0 Hz, 1H), 8.09 (s, 1H), 7.95-7.83 (m, 2H), 7.47 (d, J=7.3 Hz, 1H), 7.35 (d, J=8.2 Hz, 2H), 4.95 (s, 1H), 4.70 (dt, J=7.3, 3.7 Hz, 1H), 4.66 (dt, J=6.7, 3.3 Hz, 1H), 4.37 (d, J=6.1 Hz, 2H), 4.21 (dd, J=9.9, 4.1 Hz, 1H), 4.04-3.94 (m, 2H), 3.88-3.81 (m, 1H), 3.79 (s, 3H), 1.48 (s, 9H), 0.95 (s, 9H), 0.14 (s, 3H), 0.13 (s, 3H).

### Methyl 2-(2-(2-(4-(((tert-butoxycarbonyl)amino)methyl)phenyl)thiazole-4-carboxamido)acrylamido)acrylate (8)

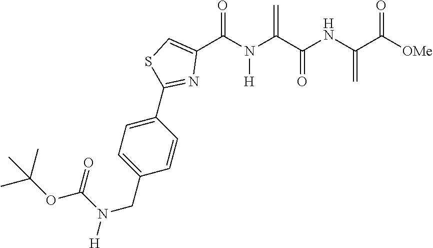 [SMILES: C=C(NC(=O)c1csc(-c2ccc(CNC(=O)OC(C)(C)C)cc2)n1)C(=O)NC(=C)C(=O)OC]

Using the procedure described for Compound 2, methyl N-(2-(4-(((tert-butoxycarbonyl)amino)methyl)-phenyl)thiazole-4-carbonyl)-O-(tert-butyldimethylsilyl)-L-seryl-L-serinate (0.263 g, 0.413 mmole) was converted to methyl 2-(2-(2-(4-(((tert-butoxycarbonyl)amino)methyl)phenyl)thiazole-4-carboxamido)acrylamido)acrylate (0.0461 g, 15%) as a white solid.

¹H NMR (400 MHz, CDCl₃) δ 10.02 (s, 1H), 8.55 (s, 1H), 8.14 (s, 1H), 8.03-7.93 (m, 2H), 7.38 (d, J=8.2 Hz, 2H), 6.78 (d, J=2.2 Hz, 1H), 6.71 (d, J=0.5 Hz, 1H), 6.03 (d, J=1.3 Hz, 1H), 5.50 (t, J=1.9 Hz, 1H), 4.92 (s, 1H), 4.38 (d, J=6.1 Hz, 2H), 3.91 (s, 3H), 1.48 (s, 9H).

### Compound 9: Tert-butyl 4-(4-((3-((3-methoxy-3-oxoprop-1-en-2-yl)amino)-3-oxoprop-1-en-2-yl)carbamoyl)thiazol-2-yl)-3,6-dihydropyridine-1(2H)-carboxylate

### Tert-butyl (S)-4-(4-((3-((tert-butyldimethylsilyl)oxy)-1-methoxy-1-oxopropan-2-yl)carbamoyl)thiazol-2-yl)-3,6-dihydropyridine-1(2H)-carboxylate (9.1)

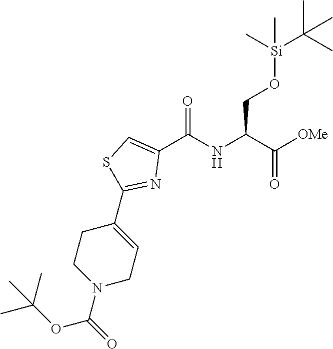 [SMILES: COC(=O)[C@H](CO[Si](C)(C)C(C)(C)C)NC(=O)c1csc(C2=CCN(C(=O)OC(C)(C)C)CC2)n1]

Methyl N-(2-bromothiazole-4-carbonyl)-O-(tert-butyldimethylsilyl)-L-serinate (0.511 g, 1.21 mmole) was dissolved in 1,4-dioxane (4 mL). Tert-butyl 4-(4,4,5,5-tetramethyl-1,3,2-dioxaborolan-2-yl)-3,6-dihydropyridine-1(2H)-carboxylate (0.440 g, 1.42 mmole) was added followed by potassium carbonate (2M aqueous solution, 1.20 mL, 2.40 mmole) and bis(triphenylphosphine)palladium(II) chloride (0.086 g, 0.123 mmole). The mixture was heated to 80° C. for 18 hours. The mixture was cooled to RT and poured into water (25 mL), then was extracted with ethyl acetate (3×10 mL). The combined organics were dried over magnesium sulfate, filtered, and concentrated. The crude residue was purified by silica gel chromatography to provide tert-butyl (S)-4-(4-((3-((tert-butyldimethylsilyl)oxy)-1-methoxy-1-oxopropan-2-yl)carbamoyl)thiazol-2-yl)-3,6-dihydropyridine-1(2H)-carboxylate as a red gel (0.556 g, 87%). ¹H NMR (400 MHz, CDCl₃) δ 8.09 (t, J=8.6 Hz, 1H), 7.99 (s, 1H), 6.57 (s, 1H), 4.82 (ddd, J=8.7, 3.3, 2.6 Hz, 1H), 4.18 (dd, J=10.0, 2.6 Hz, 1H), 4.13 (dd, J=8.5, 5.8 Hz, 3H), 3.92 (dd, J=10.1, 3.3 Hz, 1H), 3.77 (s, 3H), 3.72-3.54 (m, 2H), 2.70 (s, 2H), 1.50 (s, 9H), 0.89 (s, 9H), 0.05 (s, 3H).

### N-(2-(1-(Tert-butoxycarbonyl)-1,2,3,6-tetrahydropyridin-4-yl)thiazole-4-carbonyl)-O-(tert-butyldimethylsilyl)-L-serine (9.2)

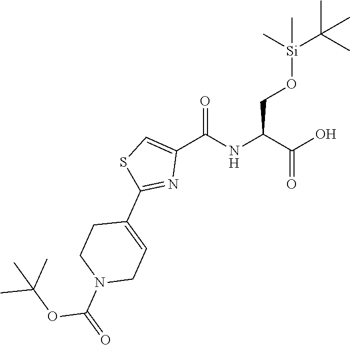 [SMILES: CC(C)(C)OC(=O)N1CC=C(c2nc(C(=O)N[C@@H](CO[Si](C)(C)C(C)(C)C)C(=O)O)cs2)CC1]

Tert-butyl (S)-4-(4-((3-((tert-butyldimethylsilyl)oxy)-1-methoxy-1-oxopropan-2-yl)carbamoyl)thiazol-2-yl)-3,6-dihydropyridine-1(2H)-carboxylate (0.556 g, 1.06 mmole) was dissolved in 4/1/1 THF/methanol/water (6 mL). Lithium hydroxide (0.086 g, 3.59 mmole) was added and the mixture was stirred at RT for 2 hours. Water (25 mL) was added and the solution was treated with 1N aq. HCl to pH=4. The slurry was extracted with ethyl acetate (3×10 mL). The combined organics were dried over sodium sulfate, filtered, and concentrated to provide N-(2-(1-(tert-butoxycarbonyl)-1,2,3,6-tetrahydropyridin-4-yl)thiazole-4-carbonyl)-O-(tert-butyldimethylsilyl)-L-serine as an orange gel (0.501 g, 92%).

¹H NMR (400 MHz, DMSO) δ 8.31 (s, 1H), 8.06 (d, J=8.7 Hz, 1H), 6.75 (d, J=14.0 Hz, 1H), 4.59 (dt, J=8.6, 3.4 Hz, 1H), 4.15-4.10 (m, 3H), 3.95 (dd, J=10.3, 3.5 Hz, 2H), 3.64-3.54 (m, 2H), 2.64 (s, 2H), 1.47 (s, 9H), 0.90 (d, J=3.7 Hz, 9H), 0.08 (s, 3H), 0.06 (s, 3H).

### Tert-butyl 4-(4-(((4S,7S)-4-(hydroxymethyl)-10,10,11,11-tetramethyl-3,6-dioxo-2,9-dioxa-5-aza-10-siladodecan-7-yl)carbamoyl)thiazol-2-yl)-3,6-dihydropyridine-1(2H)-carboxylate (9.3)

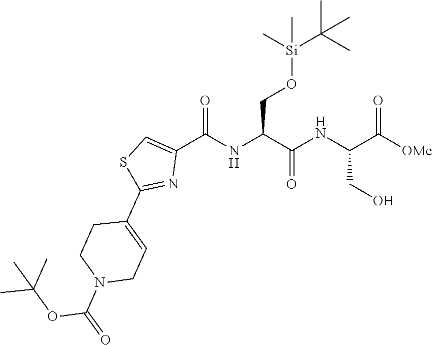 [SMILES: COC(=O)[C@H](CO)NC(=O)[C@H](CO[Si](C)(C)C(C)(C)C)NC(=O)c1csc(C2=CCN(C(=O)OC(C)(C)C)CC2)n1]

Using the procedure for Example 2.7, N-(2-(1-(tert-butoxycarbonyl)-1,2,3,6-tetrahydropyridin-4-yl)thiazole-4-carbonyl)-O-(tert-butyldimethylsilyl)-L-serine (0.501 g, 0.979 mmole) was converted to tert-butyl 4-(4-(((4S,7S)-4-(hydroxymethyl)-10,10,11,11-tetramethyl-3,6-dioxo-2,9-dioxa-5-aza-10-siladodecan-7-yl)carbamoyl)thiazol-2-yl)-3,6-dihydropyridine-1(2H)-carboxylate (0.264 g, 44%) as a white solid.

¹H NMR (400 MHz, CDCl₃) δ 8.01 (d, J=5.3 Hz, 1H), 7.46 (dd, J=13.4, 7.1 Hz, 1H), 6.57 (d, J=3.6 Hz, 1H), 4.68 (dt, J=6.9, 3.4 Hz, 1H), 4.66-4.55 (m, 1H), 4.22-4.09 (m, 2H), 3.98 (d, J=3.6 Hz, 2H), 3.82 (dt, J=10.5, 3.5 Hz, 1H), 3.80-3.73 (m, 3H), 3.61 (q, J=7.2, 6.7 Hz, 1H), 2.69 (s, 1H), 1.50 (d, J=5.6 Hz, 9H), 0.92 (s, 9H), 0.12 (d, J=3.6 Hz, 6H).

### Tert-butyl 4-(4-((3-((3-methoxy-3-oxoprop-1-en-2-yl)amino)-3-oxoprop-1-en-2-yl)carbamoyl)thiazol-2-yl)-3,6-dihydropyridine-1(2H)-carboxylate (9)

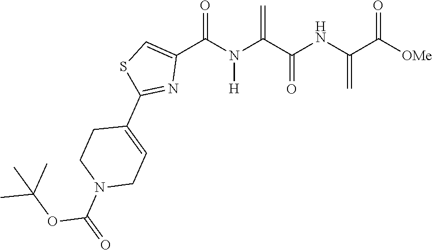 [SMILES: C=C(NC(=O)c1csc(C2=CCN(C(=O)OC(C)(C)C)CC2)n1)C(=O)NC(=C)C(=O)OC]

Using the procedure for Compound 2, tert-butyl 4-(4-(((4S,7S)-4-(hydroxymethyl)-10,10,11,11-tetramethyl-3,6-dioxo-2,9-dioxa-5-aza-10-siladodecan-7-yl)carbamoyl)thiazol-2-yl)-3,6-dihydropyridine-1(2H)-carboxylate (0.264 g, 0.431 mmole) was converted to tert-butyl 4-(4-((3-((3-methoxy-3-oxoprop-1-en-2-yl)amino)-3-oxoprop-1-en-2-yl)carbamoyl)thiazol-2-yl)-3,6-dihydropyridine-1(2H)-carboxylate (0.052 g, 27%) as a white solid.

¹H NMR (400 MHz, CDCl₃) δ 9.91 (s, 1H), 8.53 (s, 1H), 8.04 (s, 1H), 6.76 (d, J=2.2 Hz, 1H), 6.69 (s, 1H), 6.66-6.60 (m, 1H), 6.02 (d, J=1.3 Hz, 1H), 5.48 (t, J=1.9 Hz, 1H), 4.20-4.11 (m, 2H), 3.90 (s, 3H), 3.65 (t, J=5.7 Hz, 2H), 2.74 (qd, J=4.6, 4.1, 2.4 Hz, 2H), 1.49 (s, 9H).

### Compound 10: Methyl 2-(2-(2-(4-((2-methoxyethyl)carbamoyl)phenyl)thiazole-4-carboxamido)acrylamido)acrylate

### N-(2-Methoxyethyl)-4-(4,4,5,5-tetramethyl-1,3,2-dioxaborolan-2-yl)benzamide (10.1)

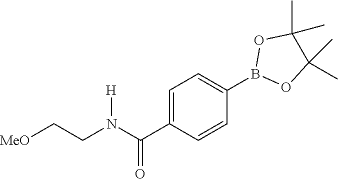 [SMILES: COCCNC(=O)c1ccc(B2OC(C)(C)C(C)(C)O2)cc1]

4-(4,4,5,5-Tetramethyl-1,3,2-dioxaborolan-2-yl)benzoic acid (0.503 g, 2.03 mmole) was dissolved in DCM (4 mL). 2-Methoxyethylamine (0.210 mL, 2.42 mmole) was added followed by N,N-diisopropylethylamine (0.700 mL, 4.02 mmole) and pyBOP (1.268 g, 2.44 mmole). The mixture was stirred at RT for 6 hours. Water (25 mL) was added and the mixture was extracted with DCM (3×10 mL). The combined organics were dried over sodium sulfate, filtered, and concentrated. The crude residue was purified by silica gel chromatography to provide N-(2-methoxyethyl)-4-(4,4,5,5-tetramethyl-1,3,2-dioxaborolan-2-yl)benzamide as a colorless oil (0.561 g, 91%).

¹H NMR (400 MHz, CDCl₃) δ 7.90-7.83 (m, 2H), 7.80-7.72 (m, 2H), 6.54 (d, J=6.2 Hz, 1H), 3.71-3.62 (m, 2H), 3.57 (dd, J=5.4, 4.3 Hz, 2H), 3.39 (s, 3H), 1.36 (s, 12H).

### Methyl O-(tert-butyldimethylsilyl)-N-(2-(4-((2-methoxyethyl)carbamoyl)phenyl)thiazole-4-carbonyl)-L-serinate (10.2)

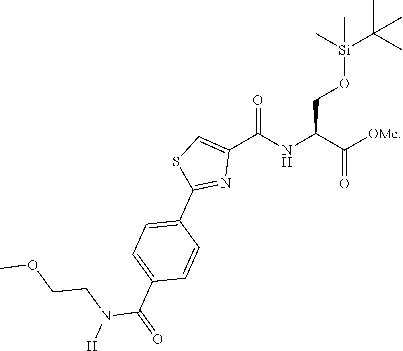 [SMILES: COCCNC(=O)c1ccc(-c2nc(C(=O)N[C@@H](CO[Si](C)(C)C(C)(C)C)C(=O)OC)cs2)cc1]

Methyl N-(2-bromothiazole-4-carbonyl)-O-(tert-butyldimethylsilyl)-L-serinate (0.656 g, 1.55 mmole) and N-(2-methoxyethyl)-4-(4,4,5,5-tetramethyl-1,3,2-dioxaborolan-2-yl)benzamide (0.561 g, 1.84 mmole) were dissolved in 1,4-dioxane (3 mL). Potassium carbonate (2M aqueous solution, 1.50 mL, 3.00 mmole) and bis(triphenylphosphine)palladium(II) chloride (0.114 g, 0.162 mmole) were added and the mixture was heated to 80° C. for 24 hours. The mixture was cooled to RT and stirred for 36 hours. Water (25 mL) was added and the mixture was extracted with ethyl acetate (3×10 mL). The combined organics were dried over magnesium sulfate, filtered, and concentrated. The crude residue was purified by silica gel chromatography to provide methyl O-(tert-butyldimethylsilyl)-N-(2-(4-((2-methoxyethyl)carbamoyl)phenyl)thiazole-4-carbonyl)-L-serinate as an orange oil (0.370 g, 46%).

¹H NMR (400 MHz, CDCl₃) δ 8.22 (d, J=8.7 Hz, 1H), 8.16 (s, 1H), 8.08-8.00 (m, 2H), 7.92-7.82 (m, 2H), 6.59 (t, J=5.2 Hz, 1H), 4.90-4.83 (m, 1H), 4.22 (dd, J=10.1, 2.6 Hz, 1H), 4.01-3.90 (m, 1H), 3.79 (s, 3H), 3.74-3.65 (m, 2H), 3.60 (dd, J=5.4, 4.3 Hz, 2H), 3.42 (s, 3H), 0.93 (s, 9H), 0.08 (s, 3H), 0.06 (s, 3H).

### O-(Tert-butyldimethylsilyl)-N-(2-(4-((2-methoxyethyl)carbamoyl)phenyl)thiazole-4-carbonyl)-L-serine (10.3)

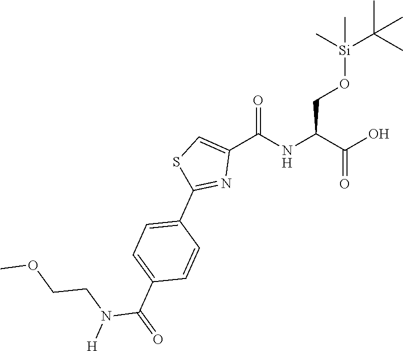 [SMILES: COCCNC(=O)c1ccc(-c2nc(C(=O)N[C@@H](CO[Si](C)(C)C(C)(C)C)C(=O)O)cs2)cc1]

Using the procedure described in Example 9.2, methyl O-(tert-butyldimethylsilyl)-N-(2-(4-((2-methoxyethyl)carbamoyl)phenyl)thiazole-4-carbonyl)-L-serinate (0.370 g, 0.709 mmole) was converted to O-(tert-butyldimethylsilyl)-N-(2-(4-((2-methoxyethyl)carbamoyl)phenyl)thiazole-4-carbonyl)-L-serine as an orange solid (0.287 g, 80%).

¹H NMR (400 MHz, DMSO-d₆) δ 8.76 (t, J=4.8 Hz, 1H), 8.49 (d, J=7.4 Hz, 1H), 8.26 (d, J=8.6 Hz, 1H), 8.16-8.10 (m, 2H), 8.10-8.01 (m, 2H), 4.65 (dt, J=8.6, 3.6 Hz, 1H), 4.16 (dd, J=10.3, 3.6 Hz, 1H), 4.01 (dd, J=10.3, 3.7 Hz, 1H), 3.57-3.45 (m, 5H), 3.44-3.35 (m, 2H), 0.93 (s, 9H), 0.11 (s, 3H), 0.09 (s, 3H).

### Methyl O-(tert-butyldimethylsilyl)-N-(2-(4-((2-methoxyethyl)carbamoyl)phenyl)thiazole-4-carbonyl)-L-seryl-L-serinate (10.4)

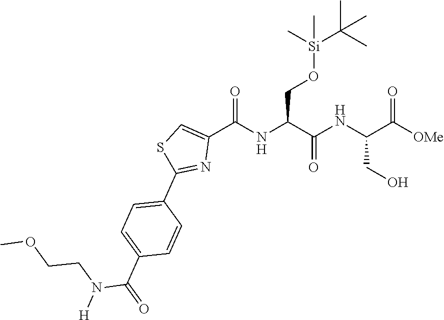 [SMILES: COCCNC(=O)c1ccc(-c2nc(C(=O)N[C@@H](CO[Si](C)(C)C(C)(C)C)C(=O)N[C@@H](CO)C(=O)OC)cs2)cc1]

Using the procedure described for Example 2.7, O-(tert-butyldimethylsilyl)-N-(2-(4-((2-methoxyethyl)carbamoyl)phenyl)thiazole-4-carbonyl)-L-serine (0.287 g, 0.565 mmole) was converted to methyl O-(tert-butyldimethylsilyl)-N-(2-(4-((2-methoxyethyl)carbamoyl)phenyl)thiazole-4-carbonyl)-L-seryl-L-serinate (0.344 g, 100%) as a white solid.

¹H NMR (400 MHz, CDCl₃) δ 8.25 (d, J=6.9 Hz, 1H), 8.17 (d, J=2.1 Hz, 1H), 8.06-7.99 (m, 2H), 7.90-7.82 (m, 2H), 7.44 (d, J=7.0 Hz, 1H), 6.57 (s, 1H), 4.70 (dt, J=7.4, 3.8 Hz, 1H), 4.67-4.60 (m, 1H), 4.22 (dd, J=9.8, 4.1 Hz, 1H), 4.04-3.95 (m, 2H), 3.89-3.81 (m, 1H), 3.79 (d, J=6.8 Hz, 3H), 3.73-3.65 (m, 2H), 3.60 (dd, J=5.4, 4.2 Hz, 2H), 3.42 (s, 3H), 0.96 (s, 9H), 0.16 (s, 3H), 0.15 (s, 3H).

### Methyl 2-(2-(2-(4-((2-methoxyethyl)carbamoyl)phenyl)thiazole-4-carboxamido)acrylamido)acrylate (10)

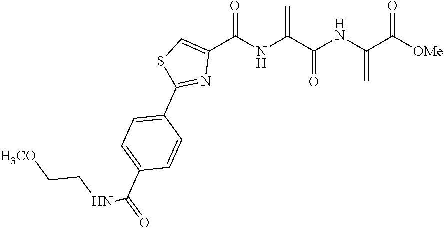 [SMILES: C=C(NC(=O)c1csc(-c2ccc(C(=O)NCCOC)cc2)n1)C(=O)NC(=C)C(=O)OC]

Using the procedure described for Compound 2, methyl (S)-2-(3-hydroxy-2-(2-(4-((2-methoxyethyl)carbamoyl)phenyl)-thiazole-4-carboxamido)propanamido)acrylate (0.212 g, 0.445 mmole) was converted to methyl 2-(2-(2-(4-((2-methoxyethyl)carbamoyl)phenyl)thiazole-4-carboxamido)acrylamido)acrylate as a white solid (0.086 g, 33%).

¹H NMR (400 MHz, CDCl₃) δ 10.02 (s, 1H), 8.56 (s, 1H), 8.20 (s, 1H), 8.13-8.05 (m, 2H), 7.94-7.83 (m, 2H), 6.79 (d, J=2.2 Hz, 1H), 6.72 (s, 1H), 6.58 (d, J=5.7 Hz, 1H), 6.04 (d, J=1.3 Hz, 1H), 5.52 (t, J=1.9 Hz, 1H), 3.91 (s, 3H), 3.75-3.65 (m, 2H), 3.60 (dd, J=5.4, 4.3 Hz, 2H), 3.42 (s, 3H).

### Compound 11: Methyl (R)-2-(2-(2-(3-((tert-butoxycarbonyl)amino)piperidin-1-yl)thiazole-4-carboxamido)acrylamido)acrylate

### Ethyl (R)-2-(3-((tert-butoxycarbonyl)amino)piperidin-1-yl)thiazole-4-carboxylate (11.1)

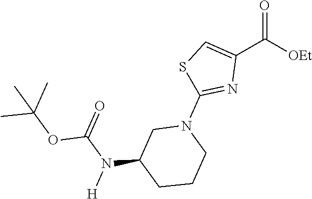 [SMILES: CCOC(=O)c1csc(N2CCC[C@@H](NC(=O)OC(C)(C)C)C2)n1]

Ethyl 2-bromothiazole-4-carboxylate (1.007 g, 4.27 mmole) was dissolved in N,N-dimethylacetamide (8 mL). Tert-butyl (R)-piperidin-3-ylcarbamate (0.900 g, 4.49 mmole) was added followed by triethylamine (0.660 mL, 4.71 mmole). The solution was heated to 70° C. for 3 days, then was cooled to RT. Water (40 mL) was added and the mixture was extracted with ethyl acetate (3×15 mL). The combined organics were dried over magnesium sulfate, filtered, and concentrated. The crude residue was purified by silica gel chromatography to provide ethyl (R)-2-(3-((tert-butoxycarbonyl)amino)piperidin-1-yl)thiazole-4-carboxylate as a yellow solid (1.032 g, 68%).

¹H NMR (400 MHz, Chloroform-d) δ 7.44 (s, 1H), 4.69 (s, 1H), 4.35 (q, J=7.1 Hz, 2H), 3.80 (s, 1H), 3.70 (d, J=12.8 Hz, 1H), 3.61 (s, 1H), 3.46 (s, 1H), 3.29 (s, 1H), 1.97-1.86 (m, 1H), 1.86-1.76 (m, 1H), 1.76-1.65 (m, 1H), 1.57 (d, J=24.5 Hz, 1H), 1.45 (s, 9H), 1.37 (t, J=7.1 Hz, 3H).

### (R)-2-(3-((Tert-butoxycarbonyl)amino)piperidin-1-yl)thiazole-4-carboxylic Acid (11.2)

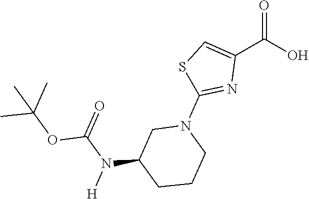 [SMILES: CC(C)(C)OC(=O)N[C@@H]1CCCN(c2nc(C(=O)O)cs2)C1]

Using the procedure described for Example 1.3, ethyl (R)-2-(3-((tert-butoxycarbonyl)amino)piperidin-1-yl)thiazole-4-carboxylate (1.032 g, 2.90 mmole) was converted to (R)-2-(3-((tert-butoxycarbonyl)amino)piperidin-1-yl)thiazole-4-carboxylic acid (0.847 g, 89%) as a yellow solid.

¹H NMR (400 MHz, CDCl₃) δ 7.51 (s, 1H), 4.66 (s, 1H), 3.86-3.71 (m, 2H), 3.59 (s, 1H), 3.40 (s, 1H), 3.26 (s, 1H), 1.95 (d, J=4.0 Hz, 1H), 1.85 (ddt, J=10.4, 7.2, 3.6 Hz, 1H), 1.72 (ddq, J=13.1, 8.6, 4.0 Hz, 1H), 1.57 (s, 1H), 1.46 (s, 9H).

### Methyl (2-((R)-3-((tert-butoxycarbonyl)amino)piperidin-1-yl)thiazole-4-carbonyl)-L-serinate (11.3)

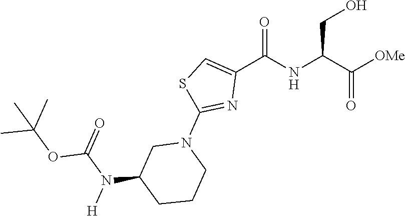 [SMILES: COC(=O)[C@H](CO)NC(=O)c1csc(N2CCC[C@@H](NC(=O)OC(C)(C)C)C2)n1]

Using the procedure described for Example 1.4, (R)-2-(3-((tert-butoxycarbonyl)amino)piperidin-1-yl)thiazole-4-carboxylic acid (0.400 g, 1.22 mmole) was converted to methyl (2-((R)-3-((tert-butoxycarbonyl)amino)piperidin-1-yl)thiazole-4-carbonyl)-L-serinate (0.547 g, 100%) as a white solid.

¹H NMR (400 MHz, CDCl₃) δ 8.05 (d, J=7.8 Hz, 1H), 7.40 (s, 1H), 4.79 (dt, J=7.6, 3.8 Hz, 1H), 4.65 (s, 1H), 4.09-3.99 (m, 2H), 3.89 (d, J=12.6 Hz, 1H), 3.81 (s, 4H), 3.54 (s, 1H), 3.39 (s, 1H), 3.13 (dd, J=12.5, 7.7 Hz, 1H), 1.93 (dd, J=7.8, 4.5 Hz, 1H), 1.71 (dp, J=13.5, 4.5 Hz, 1H), 1.56 (d, J=9.0 Hz, 1H), 1.45 (s, 9H).

### N-(2-((R)-3-((Tert-butoxycarbonyl)amino)piperidin-1-yl)thiazol-4-carbonyl)-O-(tert-butyldimethylsilyl)-L-serine (11.4)

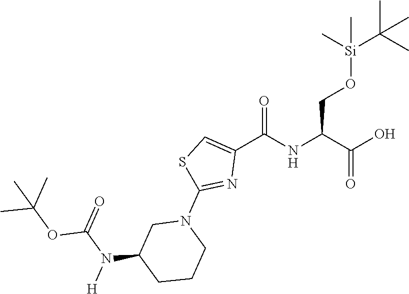 [SMILES: CC(C)(C)OC(=O)N[C@@H]1CCCN(c2nc(C(=O)N[C@@H](CO[Si](C)(C)C(C)(C)C)C(=O)O)cs2)C1]

Using the procedure described for Example 4.2 and 4.3, methyl (2-((R)-3-((tert-butoxycarbonyl)amino)piperidin-1-yl)thiazole-4-carbonyl)-L-serinate (0.456 g, 1.06 mmole) was converted to N-(2-((R)-3-((tert-butoxycarbonyl)amino)piperidin-1-yl)thiazol-4-carbonyl)-O-(tert-butyldimethylsilyl)-L-serine (0.510 g, 91%) as a colorless oil.

¹H NMR (400 MHz, CDCl₃) δ 7.95 (d, J=7.6 Hz, 1H), 7.42 (s, 1H), 4.78-4.64 (m, 2H), 4.22 (dd, J=10.1, 3.6 Hz, 1H), 3.90 (dd, J=10.1, 4.7 Hz, 1H), 3.80 (s, 1H), 3.69 (s, 1H), 3.54 (s, 1H), 3.39 (s, 1H), 3.24 (s, 1H), 1.90 (d, J=17.3 Hz, 1H), 1.71 (dt, J=9.0, 4.3 Hz, 1H), 1.45 (s, 10H), 0.91 (d, J=5.4 Hz, 9H), 0.11 (s, 3H), 0.09 (s, 3H).

### Methyl N-(2-((R)-3-((tert-butoxycarbonyl)amino)piperidin-1-yl)thiazole-4-carbonyl)-O-(tert-butyldimethylsilyl)-L-seryl-L-serinate (11.5)

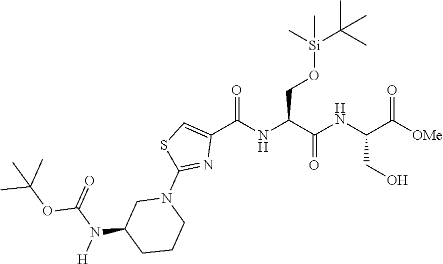 [SMILES: COC(=O)[C@H](CO)NC(=O)[C@H](CO[Si](C)(C)C(C)(C)C)NC(=O)c1csc(N2CCC[C@@H](NC(=O)OC(C)(C)C)C2)n1]

Using the procedure described for Example 2.7, N-(2-((R)-3-((tert-butoxycarbonyl)amino)piperidin-1-yl)thiazol-4-carbonyl)-O-(tert-butyldimethylsilyl)-L-serine (0.510 g, 0.965 mmole) was converted to methyl N-(2-((R)-3-((tert-butoxycarbonyl)amino)piperidin-1-yl)thiazole-4-carbonyl)-O-(tert-butyldimethylsilyl)-L-seryl-L-serinate (0.401 g, 66%) as a pale yellow oil.

¹H NMR (400 MHz, CDCl₃) δ 7.97 (d, J=7.1 Hz, 1H), 7.40 (s, 2H), 4.67 (td, J=8.3, 7.3, 4.8 Hz, 2H), 4.62-4.50 (m, 1H), 4.19-4.14 (m, 1H), 3.97 (d, J=3.7 Hz, 2H), 3.85-3.79 (m, 1H), 3.77 (d, J=2.6 Hz, 4H), 3.68 (s, 1H), 3.54 (s, 1H), 3.40 (s, 1H), 3.32-3.15 (m, 1H), 1.99-1.87 (m, 1H), 1.71 (d, J=9.8 Hz, 1H), 1.45 (s, 9H), 0.91 (s, 9H), 0.12 (s, 3H), 0.10 (s, 3H).

### Methyl (R)-2-(2-(2-(3-((tert-butoxycarbonyl)amino)piperidin-1-yl)thiazole-4-carboxamido)acrylamido)acrylate (11)

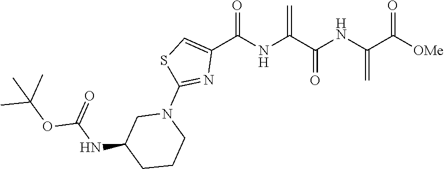 [SMILES: C=C(NC(=O)c1csc(N2CCC[C@@H](NC(=O)OC(C)(C)C)C2)n1)C(=O)NC(=C)C(=O)OC]

Using the procedure described for Compound 2, methyl 2-((S)-2-(2-((R)-3-((tert-butoxycarbonyl)amino)piperidin-1-yl)thiazole-4-carboxamido)-3-hydroxypropanamido)acrylate (0.257 g, 0.517 mmole) was converted to methyl (R)-2-(2-(2-(3-((tert-butoxycarbonyl)amino)piperidin-1-yl)thiazole-4-carboxamido)acrylamido)acrylate as a white solid (0.102 g, 33%).

¹H NMR (400 MHz, CDCl₃) δ 9.71 (s, 1H), 8.50 (s, 1H), 7.43 (s, 1H), 6.71 (d, J=2.1 Hz, 1H), 6.66 (s, 1H), 6.00 (d, J=1.3 Hz, 1H), 5.43 (t, J=1.9 Hz, 1H), 4.71 (s, 1H), 3.89 (s, 3H), 3.86-3.77 (m, 1H), 3.74-3.64 (m, 1H), 3.59 (s, 1H), 3.47 (s, 1H), 3.30 (s, 1H), 1.98-1.78 (m, 2H), 1.78-1.67 (m, 1H), 1.45 (s, 9H).

### Compound 12: Methyl 2-(2-(2-(4-((tert-butoxycarbonyl)amino)piperidin-1-yl)thiazole-4-carboxamido)acrylamido)acrylate

### Ethyl 2-(4-((tert-butoxycarbonyl)amino)piperidin-1-yl)thiazole-4-carboxylate (12.1)

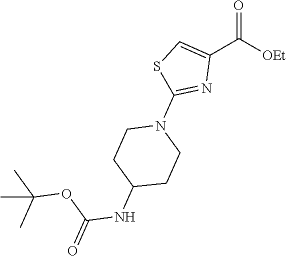 [SMILES: CCOC(=O)c1csc(N2CCC(NC(=O)OC(C)(C)C)CC2)n1]

Ethyl 2-bromothiazole-4-carboxylate (3.007 g, 12.7 mmole) was dissolved in N,N-dimethylacetamide (13 mL). Tert-butyl piperidin-4-yl carbamate (2.668 g, 13.3 mmole) was added followed by trimethylamine (2.00 mL, 14.3 mmole), and the mixture was heated to 80° C. for 24 hours. The solution was cooled to RT and water (50 mL) was added. The mixture was extracted with ethyl acetate (3×20 mL). The combined organics were dried over magnesium sulfate, filtered, and concentrated. The crude residue was purified by silica gel chromatography to provide ethyl 2-(4-((tert-butoxycarbonyl)amino)piperidin-1-yl)thiazole-4-carboxylate as a white solid (3.562 g, 79%).

¹H NMR (400 MHz, CDCl₃): δ 7.43 (s, 1H), 4.48 (s, 1H), 4.35 (q, J=7.1 Hz, 2H), 4.05-3.92 (m, 2H), 3.68 (s, 1H), 3.15 (ddd, J=13.2, 11.6, 3.0 Hz, 2H), 2.05 (m, 2H), 1.56-1.47 (m, 2H), 1.45 (s, 9H), 1.37 (t, J=7.1 Hz, 3H).

### 2-(4-((Tert-butoxycarbonyl)amino)piperidin-1-yl)thiazole-4-carboxylic Acid (12.2)

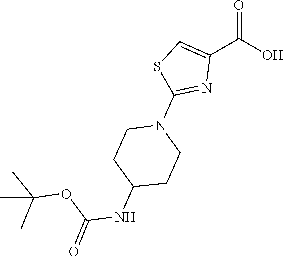 [SMILES: CC(C)(C)OC(=O)NC1CCN(c2nc(C(=O)O)cs2)CC1]

The procedure described in Example 1.3 was used to convert ethyl 2-(4-((tert-butoxycarbonyl)amino)piperidin-1-yl)thiazole-4-carboxylate (3.562 g, 10.0 mmole) to 2-(4-((tert-butoxycarbonyl)amino)piperidin-1-yl)thiazole-4-carboxylic acid as a yellow solid (2.994 g, 91%).

¹H NMR (400 MHz, CDCl₃): δ 7.52 (s, 1H), 4.50 (s, 1H), 3.95 (dt, J=13.5, 3.9 Hz, 2H), 3.79-3.62 (m, 1H), 3.18 (ddd, J=13.3, 11.6, 3.0 Hz, 2H), 2.10-2.02 (m, 2H), 1.58-1.48 (m, 2H), 1.46 (s, 9H).

### Methyl N-(2-(4-((tert-butoxycarbonyl)amino)piperidin-1-yl)thiazole-4-carbonyl)-O-(tert-butyldimethylsilyl)-L-serinate (12.3)

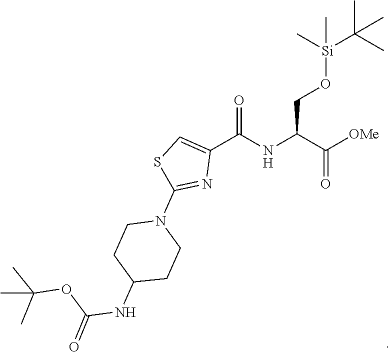 [SMILES: COC(=O)[C@H](CO[Si](C)(C)C(C)(C)C)NC(=O)c1csc(N2CCC(NC(=O)OC(C)(C)C)CC2)n1]

2-(4-((Tert-butoxycarbonyl)amino)piperidin-1-yl)thiazole-4-carboxylic acid (0.500 g, 1.53 mmole) was dissolved in DCM (3 mL). N,N′-Carbonyldiimidazole (0.262 g, 1.62 mmole) was added portionwise (gas evolution was observed) and the mixture was stirred at RT for 60 minutes. L-Serine methyl ester hydrochloride (0.262 g, 1.68 mmole) and N,N-diisopropylethylamine (0.320 mL, 1.84 mmole) were added and the solution was stirred at RT for 18 hours. Water (25 mL) was added and the mixture was extracted with DCM (3×10 mL). The combined organics were dried over magnesium sulfate, filtered, and concentrated. The residue was dissolved in DCM (5 mL). Imidazole (0.116 g, 1.70 mmole) and tert-butyldimethylchlorosilane (0.256 g, 1.70 mmole) were added and the mixture was stirred at RT for 90 minutes. Water (25 mL) was added and the mixture was extracted with DCM (3×10 mL). The combined organics were dried over magnesium sulfate, filtered, and concentrated to provide methyl N-(2-(4-((tert-butoxycarbonyl)amino)piperidin-1-yl)thiazole-4-carbonyl)-O-(tert-butyldimethylsilyl)-L-serinate as a pale yellow slurry (0.994 g).

¹H NMR (400 MHz, CDCl₃): δ 7.95 (d, J=8.8 Hz, 1H), 7.37 (s, 1H), 4.78 (ddd, J=8.8, 3.4, 2.5 Hz, 1H), 4.50 (s, 1H), 4.16 (dd, J=10.0, 2.6 Hz, 1H), 4.03-3.92 (m, 2H), 3.92-3.84 (m, 2H), 3.76 (s, 3H), 3.71 (d, J=15.5 Hz, 1H), 3.22-3.06 (m, 2H), 2.09-1.99 (m, 2H), 1.58-1.48 (m, 2H), 1.46 (s, 9H), 0.88 (s, 9H), 0.05 (s, 3H), 0.03 (s, 3H).

### N-(2-(4-((tert-butoxycarbonyl)amino)piperidin-1-yl)thiazole-4-carbonyl)-O-(tert-butyldimethylsilyl)-L-serine (12.4)

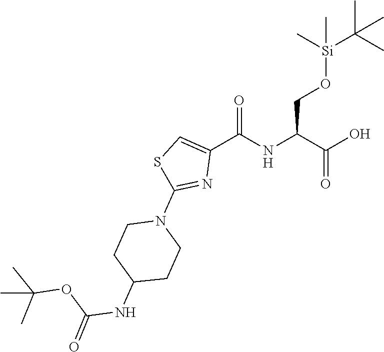 [SMILES: CC(C)(C)OC(=O)NC1CCN(c2nc(C(=O)N[C@@H](CO[Si](C)(C)C(C)(C)C)C(=O)O)cs2)CC1]

Using the procedure described in Example 4.3, methyl N-(2-(4-((tert-butoxycarbonyl)amino)piperidin-1-yl)thiazole-4-carbonyl)-O-(tert-butyldimethylsilyl)-L-serinate (0.994 g, 1.83 mmole) was converted to N-(2-(4-((tert-butoxycarbonyl)amino)piperidin-1-yl)thiazole-4-carbonyl)-O-(tert-butyldimethylsilyl)-L-serine as a yellow oil (0.900 g).

¹H NMR (400 MHz, CDCl₃): δ 7.97 (d, J=7.9 Hz, 1H), 7.41 (d, J=8.3 Hz, 1H), 4.75 (s, 1H), 4.54 (s, 1H), 4.22 (dd, J=10.0, 3.2 Hz, 1H), 3.92 (ddd, J=14.3, 8.6, 3.7 Hz, 3H), 3.80-3.63 (m, 1H), 3.22-3.06 (m, 2H), 2.08-1.99 (m, 2H), 1.42 (m, 11H), 0.90 (s, 9H), 0.08 (s, 6H).

### Methyl O-acetyl-N—(N-(2-(4-((tert-butoxycarbonyl)amino)piperidin-1-yl)thiazole-4-carbonyl)-O-(tert-butyldimethylsilyl)-L-seryl)-L-serinate (12.5)

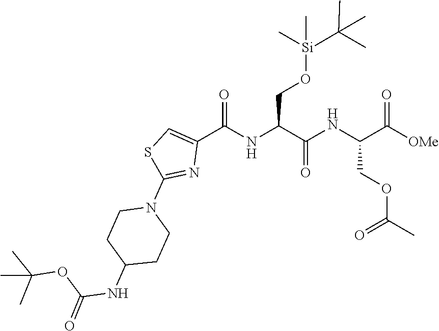 [SMILES: COC(=O)[C@H](COC(C)=O)NC(=O)[C@H](CO[Si](C)(C)C(C)(C)C)NC(=O)c1csc(N2CCC(NC(=O)OC(C)(C)C)CC2)n1]

N-(2-(4-((Tert-butoxycarbonyl)amino)piperidin-1-yl)thiazole-4-carbonyl)-O-(tert-butyldimethylsilyl)-L-serine (0.900 g, 1.70 mmole) was dissolved in DCM (4 mL) and N,N′-carbonyldiimidazole (0.306 g, 1.89 mmole) was added portionwise (gas evolution was observed). The solution was stirred at RT for 60 minutes and L-serine methyl ester hydrochloride (0.293 g, 1.88 mmole) and N,N-diisopropylethylamine (0.360 mL, 2.07 mmole) were added. The solution was stirred at RT for 2 hours and water (25 mL) was added. The mixture was extracted with DCM (3×10 mL). The combined organics were dried over magnesium sulfate, filtered, and concentrated. The residue was dissolved in DCM (4 mL) and triethylamine (0.270 mL, 1.93 mmole) and acetic anhydride (0.180 mL, 1.90 mmole) were added. The solution was stirred at RT for 18 hours and water (25 mL) was added. The mixture was extracted with DCM (3×10 mL). The combined organics were dried over magnesium sulfate, filtered, and concentrated. The crude residue was purified by silica gel chromatography to provide methyl O-acetyl-N—(N-(2-(4-((tert-butoxycarbonyl)amino)piperidin-1-yl)thiazole-4-carbonyl)-O-(tert-butyldimethylsilyl)-L-seryl)-L-serinate as a yellow oil (0.455 g, 40%).

1H NMR (400 MHz, CDCl3): δ 7.99 (d, 1H), 7.41 (d, 1H), 7.39 (s, 1H), 4.84 (m, 1H), 4.55 (m, 1H), 4.49-4.20 (m, 2H), 4.28 (dd, 1H), 4.19 (dd, 1H), 4.00-3.87 (m, 2H), 3.75 (s, 3H), 3.74-3.59 (m, 2H), 3.21-3.07 (m, 2H), 2.03 (s, 3H), 1.52-1.47 (m, 2H), 1.45 (s, 9H), 0.91 (s, 9H), 0.12 (s, 3H), 0.11 (s, 3H).

### Methyl (S)-2-(2-(2-(4-((tert-butoxycarbonyl)amino)piperidin-1-yl)thiazole-4-carboxamido)-3-hydroxypropanamido)acrylate (12.6)

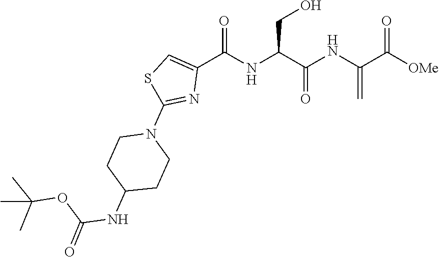 [SMILES: C=C(NC(=O)[C@H](CO)NC(=O)c1csc(N2CCC(NC(=O)OC(C)(C)C)CC2)n1)C(=O)OC]

Methyl O-acetyl-N—(N-(2-(4-((tert-butoxycarbonyl)amino)piperidin-1-yl)thiazole-4-carbonyl)-O-(tert-butyldimethylsilyl)-L-seryl)-L-serinate (0.455 g, 0.677 mmole) was dissolved in THF (1.5 mL). Tetrabutylammonium fluoride (1M solution in THF, 2.00 mL, 2.00 mmole) was added and the solution was stirred at RT for 5 hours. Water (25 mL) was added and the mixture was extracted with ethyl acetate (3×10 mL). The combined organics were dried over magnesium sulfate, filtered, and concentrated to provide methyl (S)-2-(2-(2-(4-((tert-butoxycarbonyl)amino)piperidin-1-yl)thiazole-4-carboxamido)-3-hydroxypropanamido)acrylate as a thick, yellow oil (0.347 g).

¹H NMR (400 MHz, CDCl₃): δ 9.07 (s, 1H), 8.07 (d, J=7.6 Hz, 1H), 7.43 (d, J=11.8 Hz, 1H), 6.56 (s, 1H), 5.94 (d, J=1.4 Hz, 1H), 4.70 (ddd, J=7.8, 5.1, 3.2 Hz, 1H), 4.53 (s, 1H), 4.26 (dd, J=11.5, 3.2 Hz, 1H), 4.02-3.90 (m, 3H), 3.83 (s, 3H), 3.81-3.73 (m, 1H), 3.67 (d, J=19.7 Hz, 1H), 3.21-3.07 (m, 2H), 2.07 (d, J=16.5 Hz, 2H), 1.51-1.47 (m, 2H), 1.46 (s, 9H).

### Methyl 2-(2-(2-(4-((tert-butoxycarbonyl)amino)piperidin-1-yl)thiazole-4-carboxamido)acrylamido)acrylate (12)

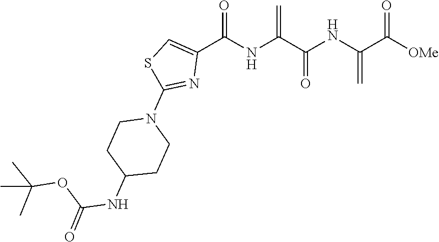 [SMILES: C=C(NC(=O)c1csc(N2CCC(NC(=O)OC(C)(C)C)CC2)n1)C(=O)NC(=C)C(=O)OC]

Methyl (S)-2-(2-(2-(4-((tert-butoxycarbonyl)amino)piperidin-1-yl)thiazole-4-carboxamido)-3-hydroxypropanamido)acrylate (0.347 g, 0.697 mmole) was dissolved in DCM (1.5 mL) and cooled to 0° C. Triethylamine (0.150 mL, 1.07 mmole) was added followed by methanesulfonyl chloride (0.080 mL, 1.03 mmole) and the mixture was stirred at 0° for 90 minutes. Water (25 mL) was added and the mixture was extracted with DCM (3×10 mL). The combined organics were dried over magnesium sulfate, filtered, and concentrated. The residue was dissolved in THF (1.5 mL) and cooled to 0° C. 1,8-Diazabicyclo[5.4.0]undec-7-ene (0.160 mL, 1.07 mmole) was added and the solution was stirred at 0° for 90 minutes. Water (25 mL) was added and the mixture was extracted with ethyl acetate (3×10 mL0. The combined organics were dried over magnesium sulfate, filtered, and concentrated. The crude residue was purified by silica gel chromatography to provide methyl 2-(2-(2-(4-((tert-butoxycarbonyl)amino)piperidin-1-yl)thiazole-4-carboxamido)acrylamido)acrylate as a white solid (0.124 g, 38% from 27.5).

¹H NMR (400 MHz, CDCl₃): δ 9.71 (s, 1H), 8.51 (s, 1H), 7.42 (s, 1H), 6.71 (d, J=2.1 Hz, 1H), 6.66 (s, 1H), 6.00 (d, J=1.3 Hz, 1H), 5.43 (t, J=1.9 Hz, 1H), 4.49 (s, 1H), 4.03-3.93 (m, 2H), 3.89 (s, 3H), 3.70 (s, 1H), 3.23-3.10 (m, 2H), 2.12-2.04 (m, 2H), 1.57-1.48 (m, 2H), 1.46 (s, 9H).

### Compound 13: Methyl 2-(2-(2-(4-(6-((tert-butoxycarbonyl)amino)hexanamido)piperidin-1-yl)thiazole-4-carboxamido)acrylamido)acrylate

### Ethyl 2-(4-(6-((tert-butoxycarbonyl)amino)hexanamido)piperidin-1-yl)thiazole-4-carboxylate (13.1)

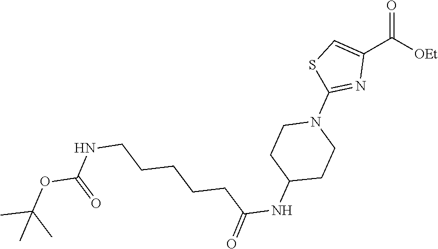 [SMILES: CCOC(=O)c1csc(N2CCC(NC(=O)CCCCCNC(=O)OC(C)(C)C)CC2)n1]

Ethyl 2-(4-((tert-butoxycarbonyl)amino)piperidin-1-yl)thiazole-4-carboxylate (Example 12.1, 7.176 g, 20.2 mmole) was dissolved in DCM (40 mL). HCl (4M solution in 1,4-dioxane, 20.0 mL, 80.0 mmole) was added and the mixture was stirred at RT for 60 minutes, then was concentrated.
In a separate flask, 6-((tert-butoxycarbonyl)amino)hexanoic acid (5.141 g, 22.2 mmole) was dissolved in DCM (40 mL). N,N′-Carbonyldiimidazole (3.765 g, 23.2 mmole) was added portionwise (gas evolution was observed) and the solution was stirred at RT for 90 minutes. The solution was added to the amine hydrochloride generated above and N,N-diisopropylethylamine (4.20 mL, 24.1 mmole) was added. The resulting solution was stirred at RT for 18 hours. Water (100 mL) was added and the two layers were separated. The aqueous layer was extracted with DCM (2×20 mL). The combined organics were dried over sodium sulfate, filtered, and concentrated. The crude residue was purified by silica gel chromatography to provide ethyl 2-(4-(6-((tert-butoxycarbonyl)amino)hexanamido)piperidin-1-yl)thiazole-4-carboxylate as a pale yellow solid (8.120 g, 86%).

¹H NMR (400 MHz, CDCl₃): δ 7.43 (d, J=1.5 Hz, 1H), 5.66 (d, J=7.9 Hz, 1H), 4.60 (s, 1H), 4.35 (q, J=7.1 Hz, 2H), 4.06-3.94 (m, 3H), 3.21-3.13 (m, 2H), 3.13-3.04 (m, 3H), 2.17 (t, J=7.6 Hz, 2H), 2.06-1.99 (m, 2H), 1.65 (p, J=7.5 Hz, 2H), 1.54-1.47 (m, 3H), 1.44 (s, 9H), 1.40-1.29 (m, 5H).

### 2-(4-(6-((Tert-butoxycarbonyl)amino)hexanamido)piperidin-1-yl)thiazole-4-carboxylic Acid (13.2)

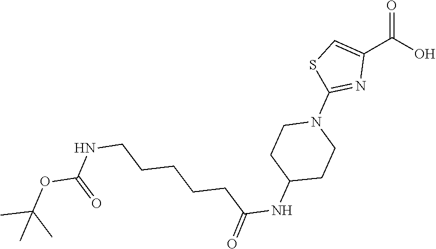 [SMILES: CC(C)(C)OC(=O)NCCCCCC(=O)NC1CCN(c2nc(C(=O)O)cs2)CC1]

Using the procedure described in Example 1.3, ethyl 2-(4-(6-((tert-butoxycarbonyl)amino)hexanamido)piperidin-1-yl)thiazole-4-carboxylate (8.120 g, 17.3 mmole) was converted to 2-(4-(6-((tert-butoxycarbonyl)amino)hexanamido)piperidin-1-yl)thiazole-4-carboxylic acid as a golden oil (9.950 g) that was carried forward without additional purification.

### Methyl N-(2-(4-(6-((tert-butoxycarbonyl)amino)hexanamido)piperidin-1-yl)thiazole-4-carbonyl)-O-(tert-butyldimethylsilyl)-L-serinate (13.3)

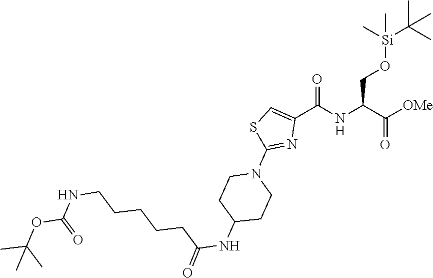 [SMILES: COC(=O)[C@H](CO[Si](C)(C)C(C)(C)C)NC(=O)c1csc(N2CCC(NC(=O)CCCCCNC(=O)OC(C)(C)C)CC2)n1]

Using the procedure described in Example 12.3, 2-(4-(6-((tert-butoxycarbonyl)amino)hexanamido)piperidin-1-yl)thiazole-4-carboxylic acid (9.950 g, 22.6 mmole) was converted to methyl N-(2-(4-(6-((tert-butoxycarbonyl)amino)hexanamido)piperidin-1-yl)thiazole-4-carbonyl)-O-(tert-butyldimethylsilyl)-L-serinate as a yellow oil (6.704 g, 59% from 29.1).

¹H NMR (400 MHz, CDCl₃): δ 7.95 (d, J=8.8 Hz, 1H), 7.38 (s, 1H), 5.60-5.49 (m, 1H), 4.78 (ddd, J=8.8, 3.3, 2.5 Hz, 1H), 4.58 (s, 1H), 4.20-4.15 (m, 1H), 4.03 (tdd, J=11.4, 7.8, 4.0 Hz, 2H), 3.97-3.90 (m, 1H), 3.88 (dd, J=10.0, 3.4 Hz, 1H), 3.76 (s, 3H), 3.21-3.13 (m, 2H), 3.11 (dq, J=7.7, 5.5, 3.7 Hz, 2H), 2.17 (t, J=7.6 Hz, 2H), 2.05 (m, 2H), 1.73-1.60 (m, 2H), 1.57-1.46 (m, 4H), 1.44 (s, 9H), 1.40-1.31 (m, 2H), 0.88 (s, 9H), 0.05 (s, 3H), 0.02 (s, 3H).

### Methyl N-(2-(4-(6-((tert-butoxycarbonyl)amino)hexanamido)piperidin-1-yl)thiazole-4-carbonyl)-O-(tert-butyldimethylsilyl)-L-seryl-L-serinate (13.4)

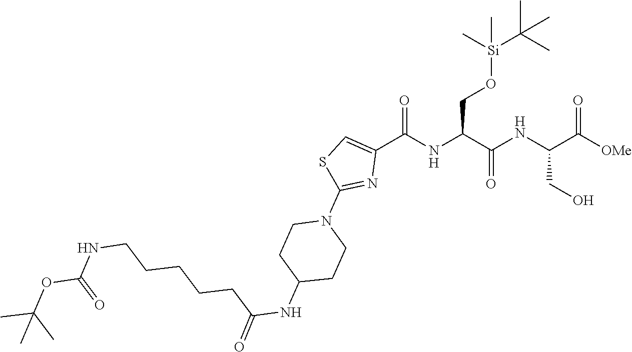 [SMILES: COC(=O)[C@H](CO)NC(=O)[C@H](CO[Si](C)(C)C(C)(C)C)NC(=O)c1csc(N2CCC(NC(=O)CCCCCNC(=O)OC(C)(C)C)CC2)n1]

Methyl N-(2-(4-(6-((tert-butoxycarbonyl)amino)hexanamido)piperidin-1-yl)thiazole-4-carbonyl)-O-(tert-butyldimethylsilyl)-L-serinate (6.704 g, 10.2 mmole) was dissolved in 4/1/1 THF/methanol/water (30 mL). Lithium hydroxide (0.738 g, 30.8 mmole) was added and the mixture was stirred at RT for 2 hours. Water (100 mL) was added and the solution was treated with 1N aq. HCl to pH=4. The mixture was extracted with diethyl ether (4×25 mL) and ethyl acetate (1×25 mL). The combined organics were dried over magnesium sulfate, filtered, and concentrated to provide N-(2-(4-(6-((tert-butoxycarbonyl)amino)hexanamido)piperidin-1-yl)thiazole-4-carbonyl)-O-(tert-butyldimethylsilyl)-L-serine. N-(2-(4-(6-((Tert-butoxycarbonyl)amino)hexanamido)piperidin-1-yl)thiazole-4-carbonyl)-O-(tert-butyldimethylsilyl)-L-serine (0.505 g, 0.787 mmole) was dissolved in DCM (1.6 mL). L-Serine methyl ester hydrochloride (0.292 g, 1.88 mmole) was added followed by N,N-diisopropylethylamine (0.270 mL, 1.55 mmole) and pyBOP (0.490 g, 0.942 mmole). The mixture was stirred at RT for 4 hours and water (25 mL) was added. The mixture was extracted with DCM (3×10 mL). The combined organics were dried over magnesium sulfate, filtered, and concentrated. The crude residue was purified by silica gel chromatography to provide methyl N-(2-(4-(6-((tert-butoxycarbonyl)amino)hexanamido)piperidin-1-yl)thiazole-4-carbonyl)-O-(tert-butyldimethylsilyl)-L-seryl-L-serinate as a thick, colorless gel (0.401 g, 69%).

1H NMR (400 MHz, CDCl₃): δ 9.07 (s, 1H), 8.05 (d, J=8.8 Hz, 1H), 7.48 (s, 1H), 6.54 (s, 1H), 5.96 (s, 1H), 5.53-5.47 (m, 1H), 4.72-4.69 (m, 1H), 4.63-4.57 (m, 2H), 4.29-4.22 (m, 1H), 4.09-3.92 (m, 4H), 3.81 (s, 3H), 3.79-3.75 (m, 1H), 3.22-3.17 (m, 2H), 3.16-3.05 (m, 2H), 2.20 (t, J=7.5 Hz, 2H), 2.05-1.97 (m, 2H), 1.70-1.59 (m, 2H), 1.51-1.40 (m, 4H), 1.43 (s, 9H), 1.38-1.27 (m, 2H).

### Methyl (S)-2-(2-(2-(4-(6-((tert-butoxycarbonyl)amino)hexanamido)piperidin-1-yl)thiazole-4-carboxamido)-3-hydroxypropanamido)acrylate (13)

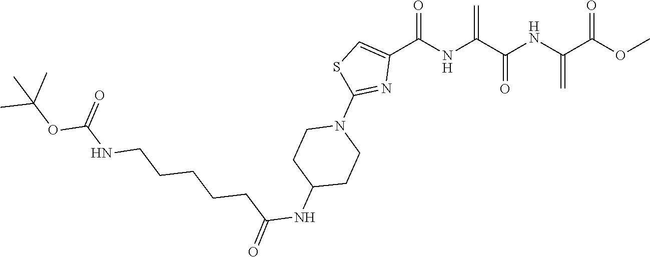 [SMILES: C=C(NC(=O)c1csc(N2CCC(NC(=O)CCCCCNC(=O)OC(C)(C)C)CC2)n1)C(=O)NC(=C)C(=O)OC]

Methyl N-(2-(4-(6-((tert-butoxycarbonyl)amino)hexanamido)piperidin-1-yl)thiazole-4-carbonyl)-O-(tert-butyldimethylsilyl)-L-seryl-L-serinate (0.401 g, 0.540 mmole) was dissolved in DCM (2 mL). Triethylamine (0.084 mL, 0.599 mmole) and acetic anhydride (0.060 mL, 0.635 mmole) were added and the mixture was stirred at RT for 18 hours. Water (25 mL) was added and the mixture was extracted with DCM (3×10 mL). The combined organics were dried over magnesium sulfate, filtered, and concentrated. The residue was dissolved in THF and tetrabutylammonium fluoride (1M solution in THF, 1.60 mL, 1.60 mmole) was added. The solution was stirred at RT for 2 hours and water (25 mL) was added. The mixture was extracted with ethyl acetate (3×10 mL). The combined organics were dried over magnesium sulfate, filtered, and concentrated to provide methyl (S)-2-(2-(2-(4-(6-((tert-butoxycarbonyl)amino)hexanamido)piperidin-1-yl)thiazole-4-carboxamido)-3-hydroxypropanamido)acrylate as a white solid (0.318 g, 96%).
Methyl (S)-2-(2-(2-(4-(6-((tert-butoxycarbonyl)amino)hexanamido)piperidin-1-yl)thiazole-4-carboxamido)-3-hydroxypropanamido)acrylate (0.318 g, 0.521 mmole) was dissolved in DCM (2 mL) and cooled to 0° C. Triethylamine (0.110 mL, 0.785 mmole) was added followed by methanesulfonyl chloride (0.060 mL, 0.775 mmole). The mixture was stirred at 0° for 2 hours and water (25 mL) was added. The mixture was extracted with DCM (3×10 mL). The combined organics were dried over magnesium sulfate, filtered, and concentrated. The residue was dissolved in THF (2 mL) and cooled to 0° C. 1,8-Diazabicyclo[5.4.0]undec-7-ene (0.120 mL, 0.802 mmole) was added and the solution was stirred at 0° for 2 hours. Water (25 mL) was added and the mixture was extracted with ethyl acetate (3×10 mL). The combined organics were dried over magnesium sulfate, filtered, and concentrated. The crude residue was purified by silica gel chromatography to provide methyl 2-(2-(2-(4-(6-((tert-butoxycarbonyl)amino)hexanamido)piperidin-1-yl)thiazole-4-carboxamido)acrylamido)acrylate as a white solid (0.106 g, 34%).

¹H NMR (400 MHz, CDCl₃): δ 9.71 (s, 1H), 8.51 (s, 1H), 7.43 (s, 1H), 6.72 (d, J=2.1 Hz, 1H), 6.66 (s, 1H), 6.00 (d, J=1.3 Hz, 1H), 5.47-5.36 (m, 2H), 4.54 (s, 1H), 4.07-3.95 (m, 3H), 3.89 (s, 3H), 3.22-3.05 (m, 4H), 2.17 (t, J=7.5 Hz, 2H), 2.10-2.00 (m, 2H), 1.66 (p, J=7.4 Hz, 2H), 1.51 (h, J=7.5 Hz, 4H), 1.43 (s, 9H), 1.40-1.30 (m, 2H).

### Compound 14: Tert-butyl 3-(4-((3-((3-methoxy-3-oxoprop-1-en-2-yl)amino)-3-oxoprop-1-en-2-yl)carbamoyl)thiazol-2-yl)benzoate

### Tert-butyl 3-carbamothioylbenzoate (14.1)

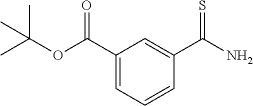 [SMILES: CC(C)(C)OC(=O)c1cccc(C(N)=S)c1]

Tert-butyl 3-cyanobenzoate, prepared as described in Adv. Synth. Catal., 2014, 356(14-15), 3074-3082, (11.887 g, 58.5 mmole) was dissolved in pyridine (60 mL). Triethylamine (9.00 mL, 64.2 mmole) was added followed by ammonium sulfide (40% aqueous solution, 12.0 mL, 70.2 mmole) and the mixture was heated to 50° C. for 4 hours. The mixture was cooled to RT and concentrated. The residue was dissolved in ethyl acetate (75 mL) and washed sequentially with 0.5N aq. HCl (3×50 mL), sat. aq. sodium bicarbonate (1×25 mL), and sat. aq. sodium chloride (1×25 mL). The organics were dried over magnesium sulfate, filtered, and concentrated to provide tert-butyl 3-carbamothioylbenzoate as a yellow solid (11.043 g, 68%).

¹H NMR (400 MHz, DMSO-d₆): δ 10.02 (s, 1H), 9.68 (s, 1H), 8.41 (td, J=1.9, 0.5 Hz, 1H), 8.07 (ddd, J=7.8, 2.0, 1.2 Hz, 1H), 8.01 (ddd, J=7.8, 1.8, 1.2 Hz, 1H), 7.55 (td, J=7.8, 0.5 Hz, 1H), 1.57 (s, 9H).

### Ethyl 2-(3-(tert-butoxycarbonyl)phenyl)thiazole-4-carboxylate (14.2)

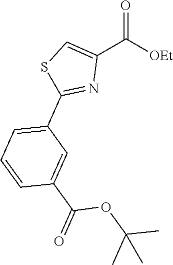 [SMILES: CCOC(=O)c1csc(-c2cccc(C(=O)OC(C)(C)C)c2)n1]

Tert-butyl 3-carbamothioylbenzoate (11.043 g, 46.5 mmole) was dissolved in 1,4-dioxane (235 mL) and cooled to 0° C. Potassium bicarbonate (37.398 g, 374 mmole) was added followed by ethyl bromopyruvate (11.7 mL, 93.2 mmole). The mixture was stirred vigorously at 0° for 2 hours, then at RT for 18 hours. The mixture was concentrated and the residue was dissolved in water (200 mL) and ethyl acetate (100 mL). The two layers were separated and the aqueous layer was extracted with ethyl acetate (2×100 mL). The combined organics were washed with sat. aq. sodium chloride (1×100 mL), dried over magnesium sulfate, filtered, and concentrated. The residue was dissolved in 1,4-dioxane (235 mL) and cooled to 0° C. Pyridine (30.0 mL, 371 mmole) was added followed by slow addition of trifluoroacetic anhydride (19.5 mL, 140 mmole) and the solution was stirred at 0° for 90 minutes, then at RT for 2 hours. The mixture was concentrated and the residue was dissolved in ethyl acetate (300 mL). The solution was washed sequentially with 1N aq. HCl (3×50 mL), sat. aq. sodium bicarbonate (1×50 mL), and sat. aq. sodium chloride (1×50 mL). The organics were dried over magnesium sulfate, filtered, and concentrated. The crude residue was split into two portions and each was purified by silica gel chromatography. The fractions containing the desired product were combined and concentrated to provide ethyl 2-(3-(tert-butoxycarbonyl)phenyl)thiazole-4-carboxylate as an orange oil (17.31 g).

¹H NMR (400 MHz, CDCl₃): δ 8.55-8.48 (m, 1H), 8.23 (ddd, J=7.8, 1.9, 1.2 Hz, 1H), 8.20 (s, 1H), 8.08 (dt, J=7.8, 1.4 Hz, 1H), 7.53 (td, J=7.8, 0.6 Hz, 1H), 4.46 (q, J=7.1 Hz, 2H), 1.63 (s, 9H), 1.44 (d, J=14.3 Hz, 3H).

### 2-(3-(tert-butoxycarbonyl)phenyl)thiazole-4-carboxylic Acid (14.3)

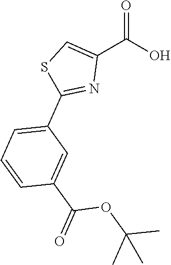 [SMILES: CC(C)(C)OC(=O)c1cccc(-c2nc(C(=O)O)cs2)c1]

Ethyl 2-(3-(tert-butoxycarbonyl)phenyl)thiazole-4-carboxylate (17.31 g, 51.9 mmole) was dissolved in 4/1/1 THF/methanol/water (200 mL). Lithium hydroxide (3.733 g, 156 mmole) was added and the mixture was stirred vigorously at RT for 2 hours. The mixture was filtered and the filtrate was stirred with additional lithium hydroxide (˜0.5 g) for 60 minutes, then was concentrated to the aqueous layer. Water (200 mL) was added and the mixture was combined with the solid above. The mixture was treated with 6N aq. HCl to pH=4. The solid was collected by filtration and washed with water, then allowed to air dry to provide 2-(3-(tert-butoxycarbonyl)phenyl)thiazole-4-carboxylic acid as a white solid (16.847 g).

¹H NMR (400 MHz, DMSO-d₆): δ 8.42 (t, J=1.7 Hz, 1H), 8.21 (ddd, J=7.8, 1.9, 1.1 Hz, 1H), 7.99 (dt, J=7.8, 1.3 Hz, 1H), 7.94 (s, 1H), 7.64 (t, J=7.8 Hz, 1H), 1.59 (s, 9H).

### Tert-butyl (S)-3-(4-((3-((tert-butyldimethylsilyl)oxy)-1-methoxy-1-oxopropan-2-yl)carbamoyl)thiazol-2-yl)benzoate (14.4)

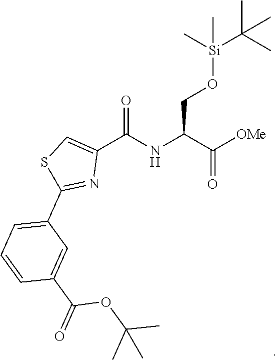 [SMILES: COC(=O)[C@H](CO[Si](C)(C)C(C)(C)C)NC(=O)c1csc(-c2cccc(C(=O)OC(C)(C)C)c2)n1]

2-(3-(Tert-butoxycarbonyl)phenyl)thiazole-4-carboxylic acid (16.847 g, 55.2 mmole) was suspended in DMF (220 mL). L-Serine methyl ester hydrochloride (10.357 g, 66.6 mmole) was added followed by 1-hydroxybenzotriazole hydrate (9.312 g, 60.8 mmole), 1-(3-dimethylaminopropyl)-3-ethylcarbodiimide hydrochloride (11.664 g, 60.8 mmole), and N,N-diisopropylethylamine (29.0 mL, 166 mmole). The mixture was stirred at RT for 18 hours and water (300 mL) and 1N aq. HCl (200 mL) were added. The mixture was extracted with ethyl acetate (4×100 mL). The combined organics were washed with sat. aq. sodium chloride (1×100 mL), dried over magnesium sulfate, filtered, and concentrated. The residue was dissolved in DCM (110 mL) and imidazole (4.160 g, 61.1 mmole) and tert-butyldimethylchlorosilane (9.158 g, 60.8 mmole) were added. The mixture was stirred at RT for 90 minutes and water (300 mL) was added. The two layers were separated and the aqueous layer was extracted with DCM (2×50 mL). The combined organics were dried over magnesium sulfate, filtered, and concentrated. The crude residue was purified by silica gel chromatography to provide tert-butyl (S)-3-(4-((3-((tert-butyldimethylsilyl)oxy)-1-methoxy-1-oxopropan-2-yl)carbamoyl)thiazol-2-yl)benzoate as a white solid (15.343 g, 63% from Intermediate 30.2).

¹H NMR (400 MHz, CDCl₃): δ 8.52-8.44 (m, 1H), 8.21 (ddd, J=7.8, 1.9, 1.2 Hz, 2H), 8.14 (s, 1H), 8.07 (dt, J=7.8, 1.4 Hz, 1H), 7.52 (td, J=7.8, 0.6 Hz, 1H), 4.87 (dt, J=8.7, 3.1 Hz, 1H), 4.21 (dd, J=10.1, 2.8 Hz, 1H), 3.96 (dd, J=10.0, 3.4 Hz, 1H), 3.79 (s, 3H), 1.64 (s, 9H), 0.91 (s, 9H), 0.08 (s, 3H), 0.06 (s, 3H).

### N-(2-(3-(tert-butoxycarbonyl)phenyl)thiazole-4-carbonyl)-O-(tert-butyldimethylsilyl)-L-serine (14.5)

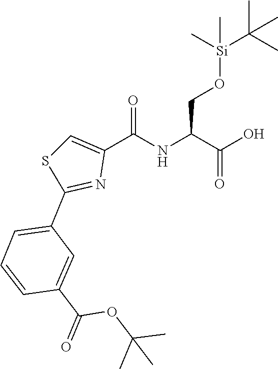 [SMILES: CC(C)(C)OC(=O)c1cccc(-c2nc(C(=O)N[C@@H](CO[Si](C)(C)C(C)(C)C)C(=O)O)cs2)c1]

Tert-butyl (S)-3-(4-((3-((tert-butyldimethylsilyl)oxy)-1-methoxy-1-oxopropan-2-yl)carbamoyl)thiazol-2-yl)benzoate (15.343 g, 29.5 mmole) was dissolved in 4/1/1 THF/methanol/water (90 mL). Lithium hydroxide (1.425 g, 59.5 mmole) was added and the mixture was stirred at RT for 3 hours. Water (300 mL) was added and the mixture was treated with 1N aq. HCl to pH=4. The mixture was extracted with ethyl acetate (4×75 mL). The combined organics were dried over magnesium sulfate, filtered, and concentrated to provide N-(2-(3-(tert-butoxycarbonyl)phenyl)thiazole-4-carbonyl)-O-(tert-butyldimethylsilyl)-L-serine as a white solid (14.560 g, 97%).

¹H NMR (400 MHz, DMSO-d₆): δ 8.46 (d, J=9.3 Hz, 2H), 8.25-8.15 (m, 2H), 8.03 (dt, J=7.8, 1.3 Hz, 1H), 7.69 (dt, J=9.7, 7.7 Hz, 1H), 4.60 (dt, J=8.4, 3.5 Hz, 1H), 4.11 (dd, J=10.3, 3.5 Hz, 1H), 3.97 (dd, J=10.4, 3.6 Hz, 1H), 1.58 (s, 9H), 0.85 (s, 9H), 0.05 (s, 3H), 0.03 (s, 3H).

### Tert-butyl 3-(4-(((4S,7S)-4-(hydroxymethyl)-10,10,11,11-tetramethyl-3,6-dioxo-2,9-dioxa-5-aza-10-siladodecan-7-yl)carbamoyl)thiazol-2-yl)benzoate (14.6)

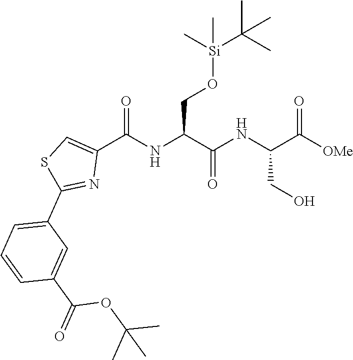 [SMILES: COC(=O)[C@H](CO)NC(=O)[C@H](CO[Si](C)(C)C(C)(C)C)NC(=O)c1csc(-c2cccc(C(=O)OC(C)(C)C)c2)n1]

N-(2-(3-(Tert-butoxycarbonyl)phenyl)thiazole-4-carbonyl)-O-(tert-butyldimethylsilyl)-L-serine (3.142 g, 6.20 mmole) was dissolved in DCM (12 mL). L-Serine methyl ester hydrochloride (1.110 g, 7.13 mmole) was added followed by N,N-diisopropylethylamine (2.10 mL, 12.1 mmole), HOBt·H2O (1.099 g, 7.18 mmole) and EDC·HCl (1.371 g, 7.15 mmole). The mixture was stirred at RT for 18 hours. Water (50 mL) was added and the two layers were separated. The aqueous layer was extracted with DCM (2×15 mL). The combined organics were dried over magnesium sulfate, filtered, and concentrated. The crude residue was purified by silica gel chromatography to provide tert-butyl 3-(4-(((4S,7S)-4-(hydroxymethyl)-10,10,11,11-tetramethyl-3,6-dioxo-2,9-dioxa-5-aza-10-siladodecan-7-yl)carbamoyl)thiazol-2-yl)benzoate as a white solid (2.159 g, 57%).

¹H NMR (400 MHz, CDCl₃): δ 8.49-8.41 (m, 1H), 8.28-8.21 (m, 1H), 8.21-8.16 (m, 1H), 8.14 (s, 1H), 8.06 (dq, J=7.8, 1.7 Hz, 1H), 7.56-7.45 (m, 2H), 4.68 (tdd, J=11.3, 8.0, 4.0 Hz, 2H), 4.20 (dt, J=9.8, 4.3 Hz, 1H), 4.05-3.94 (m, 2H), 3.90-3.82 (m, 1H), 3.78 (d, J=7.9 Hz, 3H), 1.63 (d, J=1.0 Hz, 9H), 0.94 (d, J=1.2 Hz, 9H), 0.14 (d, J=1.2 Hz, 3H), 0.14 (s, 3H), 0.13 (s, 3H).

### Tert-butyl 3-(4-(((S)-3-hydroxy-1-(((S)-3-hydroxy-1-methoxy-1-oxopropan-2-yl)amino)-1-oxopropan-2-yl)carbamoyl)thiazol-2-yl)benzoate (14.7)

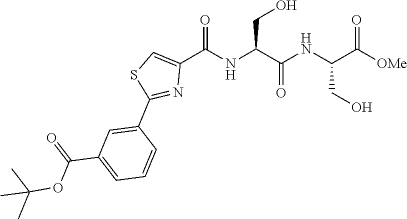 [SMILES: COC(=O)[C@H](CO)NC(=O)[C@H](CO)NC(=O)c1csc(-c2cccc(C(=O)OC(C)(C)C)c2)n1]

Tert-butyl 3-(4-(((4S,7S)-4-(hydroxymethyl)-10,10,11,11-tetramethyl-3,6-dioxo-2,9-dioxa-5-aza-10-siladodecan-7-yl)carbamoyl)thiazol-2-yl)benzoate (2.159 g, 3.55 mmole) was dissolved in THF (7 mL). Tetrabutylammonium fluoride (1M solution in THF, 3.90 mL, 3.90 mmole) was added and the solution was stirred at RT for 2½ hours. Water (50 mL) was added and the mixture was extracted with ethyl acetate (3×15 mL). The combined organics were dried over magnesium sulfate, filtered, and concentrated. The crude residue was purified by silica gel chromatography to provide tert-butyl 3-(4-(((S)-3-hydroxy-1-(((S)-3-hydroxy-1-methoxy-1-oxopropan-2-yl)amino)-1-oxopropan-2-yl)carbamoyl)thiazol-2-yl)benzoate as a white solid (1.346 g, 77%).

¹H NMR (400 MHz, CDCl₃): δ 8.44-8.36 (m, 2H), 8.17-8.09 (m, 2H), 8.03 (dq, J=7.8, 1.2 Hz, 1H), 7.90 (d, J=7.9 Hz, 1H), 7.49 (t, J=7.7 Hz, 1H), 4.92-4.80 (m, 1H), 4.75 (dq, J=7.5, 3.6 Hz, 1H), 4.17-4.10 (m, 1H), 4.05 (ddd, J=11.6, 7.4, 3.5 Hz, 2H), 3.93 (td, J=11.3, 10.1, 6.8 Hz, 1H), 3.81 (s, 3H), 1.63 (s, 9H).

### Tert-butyl 3-(4-((3-((3-methoxy-3-oxoprop-1-en-2-yl)amino)-3-oxoprop-1-en-2-yl)carbamoyl)thiazol-2-yl)benzoate (14)

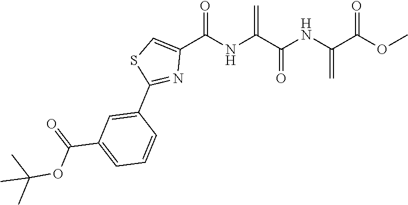 [SMILES: C=C(NC(=O)c1csc(-c2cccc(C(=O)OC(C)(C)C)c2)n1)C(=O)NC(=C)C(=O)OC]

Tert-butyl 3-(4-(((S)-3-hydroxy-1-(((S)-3-hydroxy-1-methoxy-1-oxopropan-2-yl)amino)-1-oxopropan-2-yl)carbamoyl)thiazol-2-yl)benzoate (0.200 g, 0.405 mmole) was dissolved in DCM (1 mL) and cooled to 0° C. Triethylamine (0.285 mL, 2.03 mmole) was added followed by methanesulfonyl chloride (0.160 mL, 2.07 mmole). The mixture was stirred at 0° for 60 minutes. Water (25 mL) was added and the mixture was extracted with DCM (3×10 mL). The combined organics were dried over magnesium sulfate, filtered, and concentrated. The residue was dissolved in THF and cooled to 0° C. 1,8-Diazabicyclo[5.4.0]undec-7-ene (0.300 mL, 2.01 mmole) was added and the solution was stirred at 0° for 90 minutes. Water (25 mL) was added and the mixture was extracted with ethyl acetate (3×10 mL). The combined organics were dried over magnesium sulfate, filtered, and concentrated. The crude residue was purified by silica gel chromatography to provide tert-butyl 3-(4-((3-((3-methoxy-3-oxoprop-1-en-2-yl)amino)-3-oxoprop-1-en-2-yl)carbamoyl)thiazol-2-yl)benzoate as a colorless oil (0.0338 g, 18%).

¹H NMR (400 MHz, CDCl₃): δ 10.06 (s, 1H), 8.56 (t, J=1.8 Hz, 2H), 8.24 (ddd, J=7.8, 1.9, 1.2 Hz, 1H), 8.18 (s, 1H), 8.09 (dt, J=7.8, 1.4 Hz, 1H), 7.55 (t, J=7.8 Hz, 1H), 6.78 (d, J=2.2 Hz, 1H), 6.71 (s, 1H), 6.02 (d, J=1.3 Hz, 1H), 5.51 (t, J=1.9 Hz, 1H), 3.91 (s, 3H), 1.65 (s, 9H).

### Compound 15: Methyl 2-(2-(2-(4-((2-((tert-butoxycarbonyl)amino)ethyl)carbamoyl)piperidin-1-yl)thiazole-4-carboxamido)acrylamido)acrylate

### Benzyl 4-((2-((tert-butoxycarbonyl)amino)ethyl)carbamoyl)piperidine-1-carboxylate (15.1)

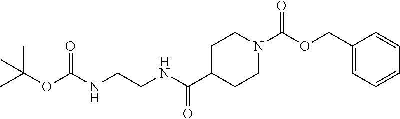 [SMILES: CC(C)(C)OC(=O)NCCNC(=O)C1CCN(C(=O)OCc2ccccc2)CC1]

1-((Benzyloxy)carbonyl)piperidine-4-carboxylic acid (2.006 g, 7.62 mmole) was dissolved in DCM (15 mL). N,N′-Carbonyldiimidazole (1.290 g, 7.96 mmole) was added portionwise (gas evolution was observed) and the solution was stirred at RT for 60 minutes. Tert-butyl (2-aminoethyl)carbamate (1.374 g, 8.58 mmole) was added slowly and the solution was stirred at RT for 2 hours. Water (50 mL) was added and the two layers were separated. The aqueous layer was extracted with DCM (2×15 mL). The combined organics were dried over magnesium sulfate, filtered, and concentrated. The crude residue was purified by silica gel chromatography to provide benzyl 4-((2-((tert-butoxycarbonyl)amino)ethyl)carbamoyl)piperidine-1-carboxylate as a white solid (2.984 g, 97%).

¹H NMR (400 MHz, CDCl₃): δ 7.41-7.28 (m, 5H), 6.43 (s, 1H), 5.12 (s, 2H), 4.92 (s, 1H), 4.20 (s, 2H), 3.40-3.23 (m, 4H), 2.83 (s, 2H), 2.24 (tt, J=11.5, 3.8 Hz, 1H), 1.84 (d, J=14.9 Hz, 2H), 1.64 (qd, J=11.7, 4.0 Hz, 2H), 1.43 (s, 9H).

### Tert-butyl (2-(piperidine-4-carboxamido)ethyl)carbamate (15.2)

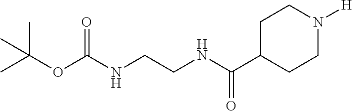 [SMILES: CC(C)(C)OC(=O)NCCNC(=O)C1CCNCC1]

Palladium on carbon (10%, Degussa type, 0.818 g, 0.769 mmole) was added to benzyl 4-((2-((tert-butoxycarbonyl)amino)ethyl)carbamoyl)piperidine-1-carboxylate (2.984 g, 7.36 mmole). Ethanol (15 mL) was added dropwise on top of the palladium to wet the catalyst, then the remaining ethanol was slowly added. Ammonium formate (0.934 g, 14.8 mmole) was added and the mixture was heated to 70° C. for 4 hours, then was cooled to RT and diluted with DCM (30 mL). The mixture was filtered through a pad of Celite and the filter cake was washed with DCM. The combined filtrates were concentrated to provide tert-butyl (2-(piperidine-4-carboxamido)ethyl)carbamate as a brown solid (2.184 g).

¹H NMR (400 MHz, CDCl₃): δ 6.40 (s, 1H), 5.08 (s, 1H), 3.40-3.31 (m, 2H), 3.31-3.22 (m, 2H), 3.12 (dt, J=12.9, 3.7 Hz, 2H), 2.61 (td, J=12.3, 2.6 Hz, 2H), 2.22 (tt, J=11.8, 3.7 Hz, 1H), 1.87-1.77 (m, 2H), 1.65-1.51 (m, 2H), 1.40 (d, J=7.0 Hz, 9H).

### Ethyl 2-(4-((2-((tert-butoxycarbonyl)amino)ethyl)carbamoyl)piperidin-1-yl)thiazole-4-carboxylate (15.3)

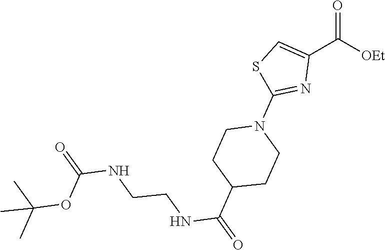 [SMILES: CCOC(=O)c1csc(N2CCC(C(=O)NCCNC(=O)OC(C)(C)C)CC2)n1]

Tert-butyl (2-(piperidine-4-carboxamido)ethyl)carbamate (2.184 g, 8.05 mmole) was dissolved in N,N-dimethylacetamide (16 mL). Ethyl 2-bromothiazole-4-carboxylate (1.913 g, 8.10 mmole) was added followed by triethylamine (1.25 mL, 8.92 mmole) and the mixture was heated to 80° C. for 24 hours. The mixture was cooled to RT and water (75 mL) was added. The mixture was extracted with ethyl acetate (3×20 mL) and the combined organics were dried over magnesium sulfate, filtered, and concentrated. The crude residue was purified by silica gel chromatography to provide ethyl 2-(4-((2-((tert-butoxycarbonyl)amino)ethyl)carbamoyl)piperidin-1-yl)thiazole-4-carboxylate as a pale yellow solid (2.174 g, 63%).

¹H NMR (400 MHz, CDCl₃): δ 7.44 (s, 1H), 6.57 (s, 1H), 4.96 (s, 1H), 4.35 (q, J=7.1 Hz, 2H), 4.08 (dt, J=13.3, 3.6 Hz, 2H), 3.41-3.32 (m, 2H), 3.29 (q, J=5.7 Hz, 2H), 3.11-3.02 (m, 2H), 2.31 (tt, J=11.5, 3.7 Hz, 1H), 2.02-1.90 (m, 2H), 1.89-1.77 (m, 2H), 1.44 (s, 9H), 1.37 (t, J=7.1 Hz, 3H).

### 2-(4-((2-((Tert-butoxycarbonyl)amino)ethyl)carbamoyl)piperidin-1-yl)thiazole-4-carboxylic Acid (15.4)

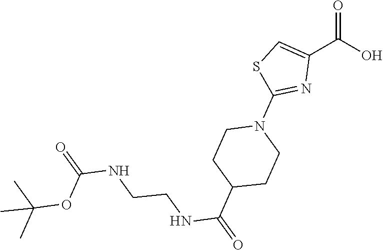 [SMILES: CC(C)(C)OC(=O)NCCNC(=O)C1CCN(c2nc(C(=O)O)cs2)CC1]

Ethyl 2-(4-((2-((tert-butoxycarbonyl)amino)ethyl)carbamoyl)piperidin-1-yl)thiazole-4-carboxylate (2.174 g, 5.10 mmole) was dissolved in 4/1/1 THF/methanol/water (15 mL). Lithium hydroxide (0.248 g, 10.4 mmole) was added and the mixture was stirred at RT for 2 hours. Water (75 mL) was added and the solution was treated with 1N aq. HCl to pH=4. The mixture was extracted with ethyl acetate (3×20 mL). A solid began to precipitate from the organics, which was collected by filtration and allowed to air dry to provide 2-(4-((2-((tert-butoxycarbonyl)amino)ethyl)carbamoyl)piperidin-1-yl)thiazole-4-carboxylic acid as a yellow solid. The filtrate was dried over magnesium sulfate, filtered, and concentrated to provide a second crop of 2-(4-((2-((tert-butoxycarbonyl)amino)ethyl)carbamoyl)piperidin-1-yl)thiazole-4-carboxylic acid. The two crops were combined (yellow solid, 1.346 g, 66%).

¹H NMR (400 MHz, DMSO-d₆): δ 12.58 (s, 1H), 7.85 (t, J=5.7 Hz, 1H), 7.61 (s, 1H), 6.79 (t, J=5.7 Hz, 1H), 3.89 (dt, J=12.9, 3.6 Hz, 2H), 3.06 (dt, J=12.2, 4.9 Hz, 4H), 3.01-2.88 (m, 2H), 2.34 (tt, J=11.3, 3.4 Hz, 1H), 1.87-1.71 (m, 2H), 1.67-1.49 (m, 2H), 1.37 (s, 9H).

### Methyl N-(2-(4-((2-((tert-butoxycarbonyl)amino)ethyl)carbamoyl)piperidin-1-yl)thiazole-4-carbonyl)-O-(tert-butyldimethylsilyl)-L-serinate (15.5)

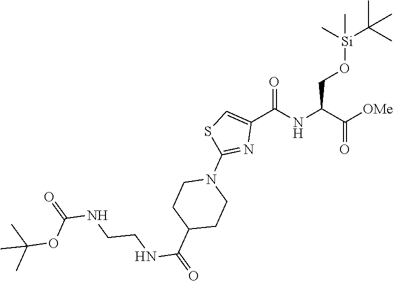 [SMILES: COC(=O)[C@H](CO[Si](C)(C)C(C)(C)C)NC(=O)c1csc(N2CCC(C(=O)NCCNC(=O)OC(C)(C)C)CC2)n1]

Using the procedure described in Example 12.3, 2-(4-((2-((tert-butoxycarbonyl)amino)ethyl)carbamoyl)piperidin-1-yl)thiazole-4-carboxylic acid (1.346 g, 3.38 mmole) was converted to methyl N-(2-(4-((2-((tert-butoxycarbonyl)amino)ethyl)carbamoyl)piperidin-1-yl)thiazole-4-carbonyl)-O-(tert-butyldimethylsilyl)-L-serinate as a thick, yellow oil (1.977 g, 95%).

¹H NMR (400 MHz, CDCl₃): δ 7.96 (d, J=8.8 Hz, 1H), 7.38 (s, 1H), 6.54 (s, 1H), 4.94 (s, 1H), 4.83-4.73 (m, 1H), 4.20-4.14 (m, 1H), 4.08-3.95 (m, 2H), 3.88 (dd, J=10.0, 3.4 Hz, 1H), 3.76 (s, 3H), 3.41-3.33 (m, 2H), 3.30 (q, J=5.8, 4.9 Hz, 2H), 3.11-2.97 (m, 2H), 2.31 (tt, J=11.5, 3.7 Hz, 1H), 1.95 (dd, J=13.4, 3.4 Hz, 2H), 1.89-1.75 (m, 2H), 1.45 (s, 9H), 0.88 (s, 9H), 0.05 (s, 3H), 0.03 (s, 3H).

### Methyl O-acetyl-N—(N-(2-(4-((2-((tert-butoxycarbonyl)amino)ethyl)carbamoyl)piperidin-1-yl)thiazole-4-carbonyl)-O-(tert-butyldimethylsilyl)-L-seryl)-L-serinate (15.6)

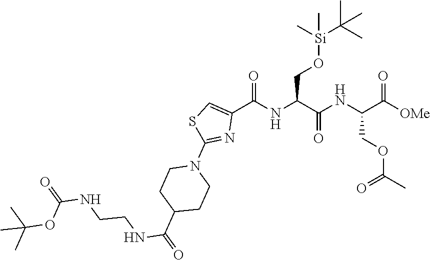 [SMILES: COC(=O)[C@H](COC(C)=O)NC(=O)[C@H](CO[Si](C)(C)C(C)(C)C)NC(=O)c1csc(N2CCC(C(=O)NCCNC(=O)OC(C)(C)C)CC2)n1]

Methyl N-(2-(4-((2-((tert-butoxycarbonyl)amino)ethyl)carbamoyl)piperidin-1-yl)thiazole-4-carbonyl)-O-(tert-butyldimethylsilyl)-L-serinate (1.977 g, 3.22 mmole) was dissolved in 4/1/1 THF/methanol/water (9 mL). Lithium hydroxide (0.157 g, 6.56 mmole) was added and the mixture was stirred at RT for 3 hours. Water (50 mL) was added and the solution was treated with 1N aq. HCl to pH=4. The mixture was extracted with ethyl acetate (3×15 mL). The combined organics were dried over magnesium sulfate, filtered, and concentrated to provide N-(2-(4-((2-((tert-butoxycarbonyl)amino)ethyl)carbamoyl)piperidin-1-yl)thiazole-4-carbonyl)-O-(tert-butyldimethylsilyl)-L-serine as a white solid (1.894 g, 98%). N-(2-(4-((2-((Tert-butoxycarbonyl)amino)ethyl)carbamoyl)piperidin-1-yl)thiazole-4-carbonyl)-O-(tert-butyldimethylsilyl)-L-serine (0.408 g, 0.680 mmole) was suspended in DCM (1.4 mL). L-Serine methyl ester hydrochloride (0.131 g, 0.842 mmole) was added followed by N,N-diisopropylethylamine (0.350 mL, 2.01 mmole), HOBt·H₂O (0.131 g, 0.855 mmole) and EDC·HCl (0.155 g, 0.809 mmole). The mixture was stirred at RT for 18 hours and water (25 mL) was added. The mixture was extracted with DCM (3×10 mL). The combined organics were dried over magnesium sulfate, filtered, and concentrated. The residue was dissolved in DCM (1.4 mL). Triethylamine (0.115 mL, 0.821 mmole) and acetic anhydride (0.076 mL, 0.804 mmole) were added and the solution was stirred at RT for 2 hours. Water (25 mL) was added and the mixture was extracted with DCM (3×10 mL). The combined organics were dried over magnesium sulfate, filtered, and concentrated. The crude residue was purified by silica gel chromatography to provide methyl O-acetyl-N—(N-(2-(4-((2-((tert-butoxycarbonyl)amino)ethyl)carbamoyl)piperidin-1-yl)thiazole-4-carbonyl)-O-(tert-butyldimethylsilyl)-L-seryl)-L-serinate as a colorless oil (0.309 g, 61%).

¹H NMR (400 MHz, CDCl₃): δ 7.99 (d, J=7.1 Hz, 1H), 7.43 (d, J=7.8 Hz, 1H), 7.39 (d, J=3.0 Hz, 1H), 6.52 (s, 1H), 4.88 (td, J=9.4, 7.7, 5.5 Hz, 2H), 4.59 (td, J=7.2, 3.6 Hz, 1H), 4.53-4.44 (m, 1H), 4.31 (dd, J=11.4, 3.7 Hz, 1H), 4.23-4.16 (m, 1H), 4.03 (dd, J=17.2, 13.6 Hz, 2H), 3.79-3.69 (m, 1H), 3.76 (s, 3H), 3.42-3.34 (m, 2H), 3.30 (q, J=5.7, 5.0 Hz, 2H), 3.04 (td, J=12.4, 3.0 Hz, 2H), 2.31 (tt, J=11.4, 3.6 Hz, 1H), 2.02 (s, 3H), 2.00-1.90 (m, 2H), 1.88-1.74 (m, 2H), 1.45 (s, 9H), 0.92 (s, 9H), 0.14 (s, 3H), 0.13 (s, 3H).

### Methyl 2-(2-(2-(4-((2-((tert-butoxycarbonyl)amino)ethyl)carbamoyl)piperidin-1-yl)thiazole-4-carboxamido)acrylamido)acrylate (15)

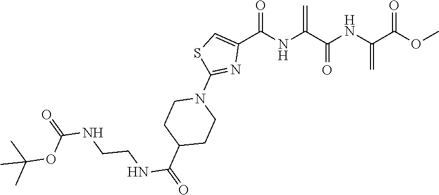 [SMILES: C=C(NC(=O)c1csc(N2CCC(C(=O)NCCNC(=O)OC(C)(C)C)CC2)n1)C(=O)NC(=C)C(=O)OC]

Methyl O-acetyl-N—(N-(2-(4-((2-((tert-butoxycarbonyl)amino)ethyl)carbamoyl)piperidin-1-yl)thiazole-4-carbonyl)-O-(tert-butyldimethylsilyl)-L-seryl)-L-serinate (0.309 g, 0.416 mmole) was dissolved in THF (1 mL). Tetrabutylammonium fluoride (1M solution in THF, 0.950 mL, 0.950 mmole) was added and the solution was stirred at RT for 3 hours. Water (25 mL) was added and the mixture was extracted with ethyl acetate (3×10 mL). The combined organics were dried over magnesium sulfate, filtered, and concentrated. The residue was dissolved in DCM (2 mL) and cooled to 0° C. Triethylamine (0.088 mL, 0.628 mmole) was added followed by methanesulfonyl chloride (0.050 mL, 0.646 mmole). The mixture was stirred at 0° for 2 hours and water (25 mL) was added. The mixture was extracted with DCM (3×10 mL). The combined organics were dried over magnesium sulfate, filtered, and concentrated. The residue was dissolved in THF (2 mL) and cooled to 0° C. 1,8-Diazabicyclo[5.4.0]undec-7-ene (0.094 mL, 0.629 mmole) was added and the solution was stirred at 0° for 2 hours. Water (25 mL) was added and the mixture was extracted with ethyl acetate (3×10 mL). The combined organics were dried over magnesium sulfate, filtered, and concentrated. The crude residue was purified by silica gel chromatography to provide methyl 2-(2-(2-(4-((2-((tert-butoxycarbonyl)amino)ethyl)carbamoyl)piperidin-1-yl)thiazole-4-carboxamido)acrylamido)acrylate as a white solid (0.046 g, 20%).

¹H NMR (400 MHz, CDCl₃): δ 9.72 (s, 1H), 8.51 (s, 1H), 7.42 (s, 1H), 6.72 (d, J=2.1 Hz, 1H), 6.66 (s, 1H), 6.49 (s, 1H), 6.00 (d, J=1.3 Hz, 1H), 5.43 (t, J=1.9 Hz, 1H), 4.88 (s, 1H), 4.06 (dt, J=13.0, 3.7 Hz, 2H), 3.89 (s, 3H), 3.43-3.34 (m, 2H), 3.32 (d, J=7.1 Hz, 2H), 3.08 (ddd, J=13.0, 11.8, 3.1 Hz, 2H), 2.32 (tt, J=11.4, 3.7 Hz, 1H), 1.99 (d, J=3.5 Hz, 2H), 1.90-1.76 (m, 2H), 1.44 (s, 9H).

### Compound 16: Methyl 2-(2-(2-(4-((6-((tert-butoxycarbonyl)amino)hexanamido)methyl)phenyl)thiazole-4-carboxamido)acrylamido)acrylate

### Tert-butyl (4-cyanobenzyl)carbamate (16.1)

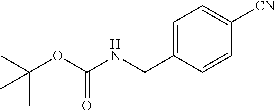 [SMILES: CC(C)(C)OC(=O)NCc1ccc(C#N)cc1]

4-(Aminomethyl)benzonitrile hydrochloride (2.010 g, 11.9 mmole) was suspended in DCM (24 mL). Di-tert-butyl dicarbonate (2.866 g, 13.1 mmole) was added followed by N,N-diisopropylethylamine (4.20 mL, 24.1 mmole) and the mixture was stirred at RT for 3 hours. Water (75 mL) was added and the two layers were separated. The aqueous layer was extracted with DCM (2×15 mL). The combined organics were dried over sodium sulfate, filtered, and concentrated to provide tert-butyl (4-cyanobenzyl)carbamate as a white solid (3.311 g).

¹H NMR (400 MHz, CDCl₃): δ 7.68-7.56 (m, 2H), 7.44-7.34 (m, 2H), 4.97 (s, 1H), 4.37 (d, J=6.2 Hz, 2H), 1.46 (s, 9H).

### Tert-butyl (4-carbamothioylbenzyl)carbamate (16.2)

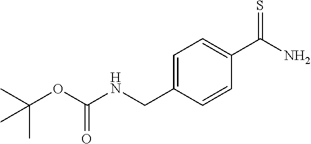 [SMILES: CC(C)(C)OC(=O)NCc1ccc(C(N)=S)cc1]

Tert-butyl (4-cyanobenzyl)carbamate (3.311 g, 14.3 mmole) was dissolved in pyridine (14 mL). Triethylamine (2.20 mL, 15.7 mmole) was added followed by ammonium sulfide (40% aqueous solution, 3.00 mL, 17.6 mmole). The solution was heated to 50° C. for 4 hours and was cooled to RT and concentrated. The residue was dissolved in ethyl acetate (50 mL) and washed sequentially with 1N aq. HCl (3×25 mL) and sat. aq. sodium chloride (1×25 mL). The organics were dried over magnesium sulfate, filtered, and concentrated to provide tert-butyl (4-carbamothioylbenzyl)carbamate as a yellow solid (3.186 g, 100% from 4-(aminomethyl)benzonitrile hydrochloride).

¹H NMR (400 MHz, DMSO-d₆): δ 9.80 (s, 1H), 9.44 (s, 1H), 7.84 (d, J=8.2 Hz, 2H), 7.44 (t, J=6.2 Hz, 1H), 7.25 (d, J=8.2 Hz, 2H), 4.15 (d, J=6.2 Hz, 2H), 1.39 (s, 9H).

### Ethyl 2-(4-(aminomethyl)phenyl)thiazole-4-carboxylate Hydrobromide (16.3)

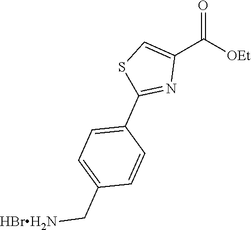

Tert-butyl (4-carbamothioylbenzyl)carbamate (2.623 g, 9.85 mmole) was dissolved in ethanol (20 mL). Ethyl bromopyruvate (1.50 mL, 12.0 mmole) was added and the mixture was heated to 80° C. for 3 hours. The mixture was cooled to RT and the precipitated solid was collected by filtration, washed with a small amount of ethanol, and allowed to air dry to provide ethyl 2-(4-(aminomethyl)phenyl)thiazole-4-carboxylate hydrobromide as a white solid (2.408 g, 71%).

¹H NMR (400 MHz, DMSO-d₆): δ 8.62 (s, 1H), 8.42-8.20 (m, 3H), 8.10-8.01 (m, 2H), 7.69-7.60 (m, 2H), 4.35 (q, J=7.1 Hz, 2H), 4.20-4.09 (m, 2H), 1.34 (t, J=7.1 Hz, 3H).

### Ethyl 2-(4-((6-((tert-butoxycarbonyl)amino)hexanamido)methyl)phenyl)thiazole-4-carboxylate (16.4)

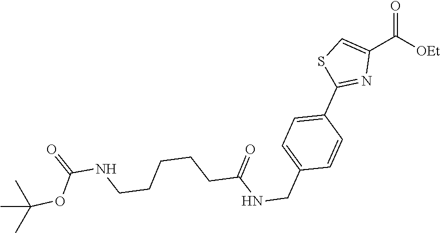 [SMILES: CCOC(=O)c1csc(-c2ccc(CNC(=O)CCCCCNC(=O)OC(C)(C)C)cc2)n1]

6-((Tert-butoxycarbonyl)amino)hexanoic acid (1.795 g, 7.76 mmole) was dissolved in DCM (14 mL). N,N′-Carbonyldiimidazole (1.254 g, 7.73 mmole) was added portionwise (gas evolution was observed) and the solution was stirred for 60 minutes. Ethyl 2-(4-(aminomethyl)phenyl)thiazole-4-carboxylate hydrobromide (2.408 g, 7.02 mmole) and N,N-diisopropylethylamine (3.70 mL, 21.2 mmole) were added and the solution was stirred at RT for 20 hours. Water (50 mL) was added and the two layers were separated. The aqueous layer was extracted with DCM (2×20 mL). The combined organics were dried over magnesium sulfate, filtered, and concentrated. The crude residue was purified by silica gel chromatography (50-100% ethyl acetate/hexane gradient) to provide ethyl 2-(4-((6-((tert-butoxycarbonyl)amino)hexanamido)methyl)phenyl)thiazole-4-carboxylate as a white solid (3.060 g, 92%).

¹H NMR (400 MHz, CDCl₃): δ 8.15 (s, 1H), 8.01-7.92 (m, 2H), 7.40-7.31 (m, 2H), 5.97 (s, 1H), 4.56 (s, 1H), 4.48 (d, J=5.9 Hz, 2H), 4.45 (q, J=7.1 Hz, 2H), 3.10 (q, J=6.7 Hz, 2H), 2.25 (t, J=7.5 Hz, 2H), 1.76-1.64 (m, 2H), 1.50 (ddd, J=14.3, 7.4, 3.9 Hz, 2H), 1.45-1.40 (m, 12H), 1.40-1.31 (m, 2H).

### 2-(4-((6-((Tert-butoxycarbonyl)amino)hexanamido)methyl)phenyl)thiazole-4-carboxylic Acid (16.5)

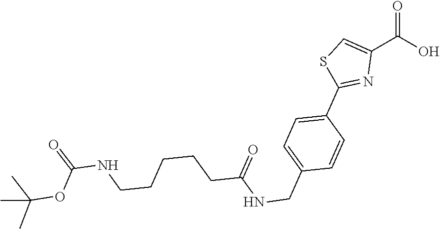 [SMILES: CC(C)(C)OC(=O)NCCCCCC(=O)NCc1ccc(-c2nc(C(=O)O)cs2)cc1]

Using the procedure described in Example 1.3, ethyl 2-(4-((6-((tert-butoxycarbonyl)amino)hexanamido)methyl)phenyl)thiazole-4-carboxylate (3.060 g, 6.43 mmole) was converted to 2-(4-((6-((tert-butoxycarbonyl)amino)hexanamido)methyl)phenyl)thiazole-4-carboxylic acid as a white solid (2.756 g, 96%).

¹H NMR (400 MHz, DMSO-d₆): δ 8.48 (s, 1H), 8.39 (t, J=6.0 Hz, 1H), 7.94 (d, J=8.3 Hz, 2H), 7.43-7.33 (m, 2H), 6.76 (t, J=5.8 Hz, 1H), 4.32 (d, J=5.9 Hz, 2H), 2.90 (q, J=6.6 Hz, 2H), 2.15 (t, J=7.4 Hz, 2H), 1.53 (p, J=7.5 Hz, 2H), 1.37 (s, 11H), 1.24 (td, J=8.4, 4.3 Hz, 2H).

### Methyl N-(2-(4-((6-((tert-butoxycarbonyl)amino)hexanamido)methyl)phenyl)thiazole-4-carbonyl)-O-(tert-butyldimethylsilyl)-L-serinate (16.6)

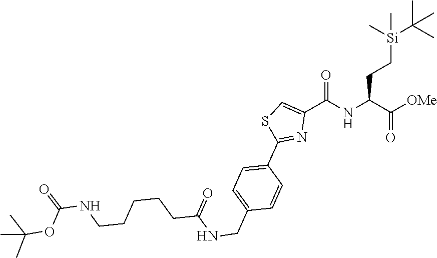 [SMILES: COC(=O)[C@H](CC[Si](C)(C)C(C)(C)C)NC(=O)c1csc(-c2ccc(CNC(=O)CCCCCNC(=O)OC(C)(C)C)cc2)n1]

Using the procedure described in Example 12.3, 2-(4-((6-((tert-butoxycarbonyl)amino)hexanamido)methyl)phenyl)thiazole-4-carboxylic acid (2.756 g, 6.16 mmole) was converted to methyl N-(2-(4-((6-((tert-butoxycarbonyl)amino)hexanamido)methyl)phenyl)thiazole-4-carbonyl)-O-(tert-butyldimethylsilyl)-L-serinate as a colorless oil (3.291 g, 77%).

¹H NMR (400 MHz, CDCl₃): δ 8.21 (d, J=8.7 Hz, 1H), 8.07 (s, 1H), 7.96-7.87 (m, 2H), 7.40-7.31 (m, 2H), 6.01 (s, 1H), 4.90-4.80 (m, 1H), 4.57 (s, 1H), 4.49 (d, J=5.8 Hz, 2H), 4.21 (dd, J=10.0, 2.7 Hz, 1H), 3.95 (dd, J=10.1, 3.4 Hz, 1H), 3.79 (s, 3H), 3.11 (q, J=6.7 Hz, 2H), 2.26 (t, J=7.5 Hz, 2H), 1.78-1.66 (m, 2H), 1.56-1.47 (m, 2H), 1.43 (s, 9H), 1.41-1.33 (m, 2H), 0.92 (s, 9H), 0.08 (s, 3H), 0.06 (s, 3H).

### N-(2-(4-((6-((tert-butoxycarbonyl)amino)hexanamido)methyl)phenyl)thiazole-4-carbonyl)-O-(tert-butyldimethylsilyl)-L-serine (16.7)

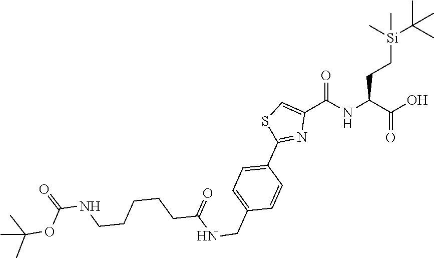 [SMILES: CC(C)(C)OC(=O)NCCCCCC(=O)NCc1ccc(-c2nc(C(=O)N[C@@H](CC[Si](C)(C)C(C)(C)C)C(=O)O)cs2)cc1]

Using the procedure described in Example 4.3, methyl N-(2-(4-((6-((tert-butoxycarbonyl)amino)hexanamido)methyl)phenyl)thiazole-4-carbonyl)-O-(tert-butyldimethylsilyl)-L-serinate (3.291 g, 4.96 mmole) was converted to N-(2-(4-((6-((tert-butoxycarbonyl)amino)hexanamido)methyl)phenyl)thiazole-4-carbonyl)-O-(tert-butyldimethylsilyl)-L-serine as a white solid (3.097 g, 96%).

¹H NMR (400 MHz, DMSO-d₆): δ 8.42-8.36 (m, 1H), 8.37 (s, 1H), 8.18 (d, J=8.6 Hz, 1H), 8.00-7.90 (m, 2H), 7.42-7.35 (m, 2H), 6.77 (d, J=6.1 Hz, 1H), 4.59 (dt, J=8.6, 3.5 Hz, 1H), 4.33 (d, J=5.9 Hz, 2H), 4.12 (dd, J=10.3, 3.4 Hz, 1H), 3.96 (dd, J=10.4, 3.7 Hz, 1H), 2.89 (q, J=6.6 Hz, 2H), 2.15 (t, J=7.4 Hz, 2H), 1.53 (p, J=7.5 Hz, 2H), 1.37 (s, 11H), 1.31-1.20 (m, 2H), 0.88 (s, 9H), 0.06 (s, 3H), 0.04 (s, 3H).

### Methyl N-(2-(4-((6-((tert-butoxycarbonyl)amino)hexanamido)methyl)phenyl)thiazole-4-carbonyl)-O-(tert-butyldimethylsilyl)-L-seryl-L-serinate (16.8)

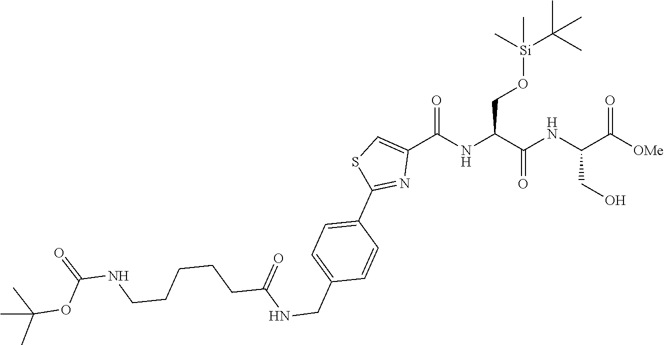 [SMILES: COC(=O)[C@H](CO)NC(=O)[C@H](CO[Si](C)(C)C(C)(C)C)NC(=O)c1csc(-c2ccc(CNC(=O)CCCCCNC(=O)OC(C)(C)C)cc2)n1]

N-(2-(4-((6-((Tert-butoxycarbonyl)amino)hexanamido)methyl)phenyl)thiazole-4-carbonyl)-O-(tert-butyldimethylsilyl)-L-serine (3.097 g, 4.77 mmole) was dissolved in DCM (9 mL). L-Serine methyl ester hydrochloride (0.898 g, 5.77 mmole) was added followed by N,N-diisopropylethylamine (1.70 mL, 9.76 mmole), HOBt·H₂O (0.881 g, 5.75 mmole), and EDC·HCl (1.101 g, 5.74 mmole). The mixture was stirred at RT for 18 hours. Water (50 mL) was added and the two layers were separated. The aqueous layer was extracted with DCM (2×15 mL). The combined organics were dried over magnesium sulfate, filtered, and concentrated. The crude residue was purified by silica gel chromatography (80-100% ethyl acetate/hexane gradient) to provide methyl N-(2-(4-((6-((tert-butoxycarbonyl)amino)hexanamido)methyl)phenyl)thiazole-4-carbonyl)-O-(tert-butyldimethylsilyl)-L-seryl-L-serinate as a white solid (2.354 g, 66%).

¹H NMR (400 MHz, CDCl₃): δ 8.24 (d, J=7.1 Hz, 1H), 8.06 (s, 1H), 7.89-7.84 (m, 2H), 7.52 (d, J=7.5 Hz, 1H), 7.35-7.29 (m, 2H), 6.15-6.02 (m, 1H), 4.74-4.63 (m, 2H), 4.61 (d, J=13.7 Hz, 1H), 4.48 (d, J=6.1 Hz, 2H), 4.19 (dd, J=9.8, 4.1 Hz, 1H), 4.05-3.95 (m, 2H), 3.88-3.82 (m, 1H), 3.78 (d, J=9.3 Hz, 4H), 3.11 (q, J=6.7 Hz, 3H), 2.26 (t, J=7.6 Hz, 2H), 1.75-1.66 (m, 3H), 1.55-1.46 (m, 2H), 1.43 (s, 10H), 1.41-1.32 (m, 2H), 0.94 (d, J=1.1 Hz, 9H), 0.14 (s, 3H), 0.12 (s, 3H).

### Methyl (2-(4-((6-((tert-butoxycarbonyl)amino)hexanamido)methyl)phenyl)thiazole-4-carbonyl)-L-seryl-L-serinate (16.9)

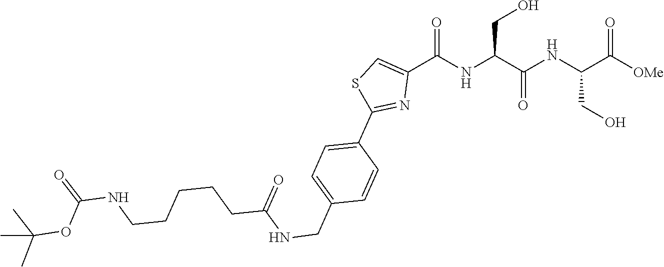 [SMILES: COC(=O)[C@H](CO)NC(=O)[C@H](CO)NC(=O)c1csc(-c2ccc(CNC(=O)CCCCCNC(=O)OC(C)(C)C)cc2)n1]

N-(2-(4-((6-((Tert-butoxycarbonyl)amino)hexanamido)methyl)phenyl)thiazole-4-carbonyl)-O-(tert-butyldimethylsilyl)-L-seryl-L-serinate (2.354 g, 3.14 mmole) was dissolved in THF (6 mL). Tetrabutylammonium fluoride (1M solution in THF, 3.50 mL, 3.50 mmole) was added and the solution was stirred at RT for 2 hours. Water (50 mL) was added and the precipitated solid was collected by filtration, washed with water and allowed to air dry to provide methyl (2-(4-((6-((tert-butoxycarbonyl)amino)hexanamido)methyl)phenyl)thiazole-4-carbonyl)-L-seryl-L-serinate as a white solid (1.790 g, 90%).

¹H NMR (400 MHz, DMSO-d₆): δ 8.49-8.41 (m, 2H), 8.38 (d, J=2.7 Hz, 1H), 8.31 (dd, J=8.1, 2.6 Hz, 1H), 8.08-7.99 (m, 2H), 7.49-7.40 (m, 2H), 6.80 (d, J=6.0 Hz, 1H), 4.68 (dt, J=8.2, 5.2 Hz, 1H), 4.46 (dt, J=7.8, 4.6 Hz, 1H), 4.37 (d, J=6.0 Hz, 2H), 3.86-3.73 (m, 3H), 3.68 (d, J=4.2 Hz, 4H), 2.93 (q, J=6.6 Hz, 2H), 2.19 (t, J=7.4 Hz, 2H), 1.57 (p, J=7.5 Hz, 2H), 1.41 (s, 11H), 1.33-1.22 (m, 2H).

### Methyl 2-(2-(2-(4-((6-((tert-butoxycarbonyl)amino)hexanamido)methyl)phenyl)thiazole-4-carboxamido)acrylamido)acrylate (16)

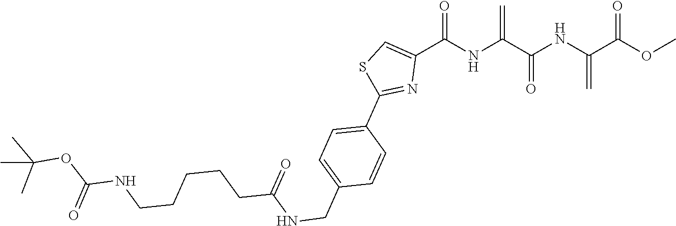 [SMILES: C=C(NC(=O)c1csc(-c2ccc(CNC(=O)CCCCCNC(=O)OC(C)(C)C)cc2)n1)C(=O)NC(=C)C(=O)OC]

Methyl (2-(4-((6-((tert-butoxycarbonyl)amino)hexanamido)methyl)phenyl)thiazole-4-carbonyl)-L-seryl-L-serinate (0.306 g, 0.481 mmole) was dissolved in DMF (2 mL) and cooled to 0° C. Triethylamine (0.200 mL, 1.43 mmole) was added followed by methanesulfonyl chloride (0.110 mL, 1.42 mmole). The mixture was stirred at 0° for 60 minutes and water (25 mL) was added. The mixture was extracted with ethyl acetate (3×10 mL). The combined organics were dried over magnesium sulfate, filtered, and concentrated. The residue was suspended in THF and cooled to 0° C. 1,8-Diazabicyclo[5.4.0]undec-7-ene (0.210 mL, 1.40 mmole) was added and the mixture was stirred at 0° for 60 minutes. Water (25 mL) was added and the mixture was extracted with ethyl acetate (3×10 mL). The combined organics were dried over magnesium sulfate, filtered, and concentrated. The crude residue was purified by silica gel chromatography (80-100% ethyl acetate/hexane gradient) to provide a white solid. The solid was triturated with ethyl acetate and collected by filtration to provide methyl 2-(2-(2-(4-((6-((tert-butoxycarbonyl)amino)hexanamido)methyl)phenyl)thiazole-4-carboxamido)acrylamido)acrylate as a white solid (0.014 g, 4.9%).

¹H NMR (400 MHz, DMSO-d₆): δ 9.97 (s, 1H), 9.95 (s, 1H), 8.48 (s, 1H), 8.38 (m, 1H), 8.02-7.91 (m, 2H), 7.46-7.36 (m, 2H), 6.76 (s, 1H), 6.51 (d, J=1.3 Hz, 1H), 5.87 (d, J=1.4 Hz, 1H), 5.87 (s, 1H), 5.84 (s, 1H), 4.32 (d, J=5.9 Hz, 2H), 3.75 (s, 3H), 2.89 (q, J=6.6 Hz, 2H), 2.15 (t, J=7.5 Hz, 2H), 1.53 (p, J=7.5 Hz, 2H), 1.36 (s, 11H), 1.24 (q, J=8.4, 7.6 Hz, 2H).

### Compound 17: Methyl 2-(2-(2-morpholinothiazole-4-carboxamido)acrylamido)acrylate

### Ethyl 2-morpholinothiazole-4-carboxylate (17.1)

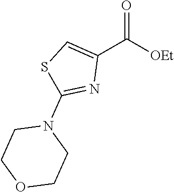 [SMILES: CCOC(=O)c1csc(N2CCOCC2)n1]

Ethyl 2-bromothiazole-4-carboxylate (1.009 g, 4.27 mmole) was dissolved in N,N-dimethylacetamide (8 mL). Morpholine (0.410 mL, 4.69 mmole) and triethylamine (0.710 mL, 5.07 mmole) were added and the mixture was heated to 80° C. for 24 hours then was cooled to RT. Water (50 mL) was added and the mixture was extracted with ethyl acetate (3×15 mL). The combined organics were dried over magnesium sulfate, filtered, and concentrated. The crude residue was purified by silica gel chromatography (20-40% ethyl acetate/hexane gradient) to provide ethyl 2-morpholinothiazole-4-carboxylate as a pale yellow solid (0.721 g, 70%).

¹H NMR (400 MHz, CDCl₃): δ 7.48 (s, 1H), 4.36 (q, J=7.1 Hz, 2H), 3.86-3.77 (m, 4H), 3.57-3.47 (m, 4H), 1.37 (t, J=7.2 Hz, 3H).

### 2-Morpholinothiazole-4-carboxylic Acid (17.2)

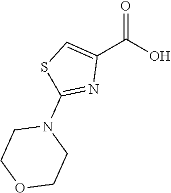 [SMILES: O=C(O)c1csc(N2CCOCC2)n1]

Using the procedure described in Example 1.3, ethyl 2-morpholinothiazole-4-carboxylate (0.721 g, 2.96 mmole) was converted to 2-morpholinothiazole-4-carboxylic acid as a white solid (0.341 g, 54%).

¹H NMR (400 MHz, DMSO-d₆): δ 12.62 (s, 1H), 7.67 (s, 1H), 3.74-3.64 (m, 4H), 3.39 (ddd, J=6.3, 4.9, 3.5 Hz, 4H).

### Methyl O-(tert-butyldimethylsilyl)-N-(2-morpholinothiazole-4-carbonyl)-L-serinate (17.3)

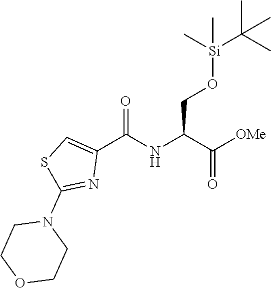 [SMILES: COC(=O)[C@H](CO[Si](C)(C)C(C)(C)C)NC(=O)c1csc(N2CCOCC2)n1]

Using the procedure described in Example 12.3, 2-morpholinothiazole-4-carboxylic acid (0.341 g, 1.59 mmole) was converted to methyl O-(tert-butyldimethylsilyl)-N-(2-morpholinothiazole-4-carbonyl)-L-serinate as a colorless oil (0.494 g, 72%).

¹H NMR (400 MHz, CDCl₃): δ 7.95 (d, J=8.8 Hz, 1H), 7.43 (s, 1H), 4.79 (ddd, J=8.8, 3.4, 2.6 Hz, 1H), 4.20-4.13 (m, 1H), 3.85-3.79 (m, 6H), 3.76 (s, 3H), 3.56-3.50 (m, 2H), 3.48 (q, J=4.9 Hz, 4H), 0.88 (s, 9H), 0.04 (s, 3H), 0.03 (s, 3H).

### O-(tert-butyldimethylsilyl)-N-(2-morpholinothiazole-4-carbonyl)-L-serine (17.4)

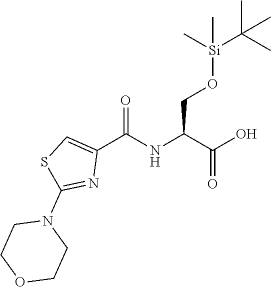 [SMILES: CC(C)(C)[Si](C)(C)OC[C@H](NC(=O)c1csc(N2CCOCC2)n1)C(=O)O]

Using the procedure described in Example 4.3, methyl O-(tert-butyldimethylsilyl)-N-(2-morpholinothiazole-4-carbonyl)-L-serinate (0.494 g, 1.15 mmole) was converted to O-(tert-butyldimethylsilyl)-N-(2-morpholinothiazole-4-carbonyl)-L-serine as a colorless gel (0.396 g, 83%).

¹H NMR (400 MHz, DMSO-d₆): δ 7.87 (d, J=8.6 Hz, 1H), 7.56 (d, J=6.1 Hz, 1H), 4.53 (dt, J=8.7, 3.3 Hz, 1H), 4.13-4.08 (m, 1H), 3.91 (dd, J=10.3, 3.6 Hz, 1H), 3.81-3.71 (m, 4H), 3.51-3.41 (m, 4H), 0.89 (d, J=1.9 Hz, 9H), 0.07 (s, 3H), 0.06 (s, 3H).

### Methyl O-acetyl-N—(O-(tert-butyldimethylsilyl)-N-(2-morpholinothiazole-4-carbonyl)-L-seryl)-L-serinate (17.5)

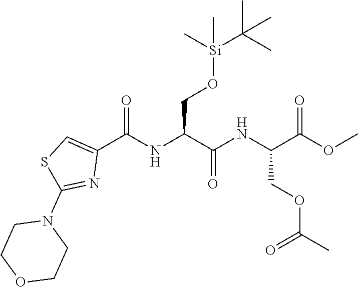 [SMILES: COC(=O)[C@H](COC(C)=O)NC(=O)[C@H](CO[Si](C)(C)C(C)(C)C)NC(=O)c1csc(N2CCOCC2)n1]

Using the procedure described in Example 15.6, O-(tert-butyldimethylsilyl)-N-(2-morpholinothiazole-4-carbonyl)-L-serine (0.396 g, 0.953 mmole) was converted to methyl O-acetyl-N—(O-(tert-butyldimethylsilyl)-N-(2-morpholinothiazole-4-carbonyl)-L-seryl)-L-serinate as a colorless oil (0.339 g, 64%).

¹H NMR (400 MHz, CDCl₃): δ 7.99 (d, J=7.1 Hz, 1H), 7.43 (d, J=2.2 Hz, 2H), 4.91-4.82 (m, 1H), 4.63-4.54 (m, 1H), 4.53-4.41 (m, 1H), 4.35 (ddd, J=26.4, 11.4, 3.7 Hz, 1H), 4.18 (ddd, J=9.8, 3.7, 0.9 Hz, 1H), 3.87-3.79 (m, 5H), 3.76 (s, 3H), 3.53-3.44 (m, 4H), 2.03 (s, 3H), 0.92 (d, J=1.0 Hz, 9H), 0.15 (s, 3H), 0.12 (s, 3H).

### Methyl 2-(2-(2-morpholinothiazole-4-carboxamido)acrylamido)acrylate (17)

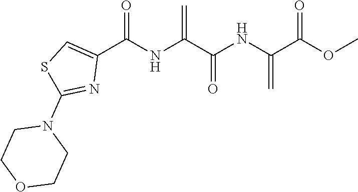 [SMILES: C=C(NC(=O)c1csc(N2CCOCC2)n1)C(=O)NC(=C)C(=O)OC]

Using the procedure described in Compound 15, methyl O-acetyl-N—(O-(tert-butyldimethylsilyl)-N-(2-morpholinothiazole-4-carbonyl)-L-seryl)-L-serinate (0.339 g, 0.607 mmole) was converted to methyl 2-(2-(2-morpholinothiazole-4-carboxamido)acrylamido)acrylate as a white solid (0.093 g, 42%).

¹H NMR (400 MHz, CDCl₃): δ 9.74 (s, 1H), 8.51 (s, 1H), 7.47 (s, 1H), 6.73 (d, J=2.2 Hz, 1H), 6.65 (s, 1H), 6.00 (d, J=1.3 Hz, 1H), 5.44 (t, J=1.9 Hz, 1H), 3.89 (s, 3H), 3.86-3.77 (m, 4H), 3.57-3.46 (m, 4H).

### Compound 18: Tert-butyl 4-(4-((3-((3-methoxy-3-oxoprop-1-en-2-yl)amino)-3-oxoprop-1-en-2-yl)carbamoyl)thiazol-2-yl)piperazine-1-carboxylate

### Ethyl 2-(4-(tert-butoxycarbonyl)piperazin-1-yl)thiazole-4-carboxylate (18.1)

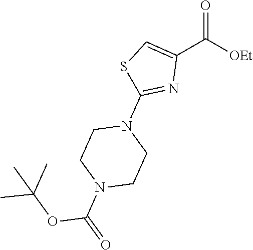 [SMILES: CCOC(=O)c1csc(N2CCN(C(=O)OC(C)(C)C)CC2)n1]

Ethyl 2-bromothiazole-4-carboxylate (1.001 g, 4.24 mmole) was dissolved in N,N-dimethylacetamide (8 mL). Tert-butyl piperazine-1-carboxylate (1.182 g, 6.35 mmole) was added followed by trimethylamine (0.720 mL, 5.14 mmole), and the mixture was heated to 80° C. for 3 days, then was cooled to RT. Water (50 mL) was added and the mixture was extracted with ethyl acetate (3×15 mL). The combined organics were dried over magnesium sulfate, filtered, and concentrated. The crude residue was purified by silica gel chromatography (10-40% ethyl acetate/hexane gradient) to provide ethyl 2-(4-(tert-butoxycarbonyl)piperazin-1-yl)thiazole-4-carboxylate as a pale yellow solid (1.325 g, 92%).

¹H NMR (400 MHz, CDCl₃): δ 7.47 (s, 1H), 4.36 (q, J=7.1 Hz, 2H), 3.59-3.47 (m, 8H), 1.48 (s, 9H), 1.37 (t, J=7.1 Hz, 3H).

### 2-(4-(Tert-butoxycarbonyl)piperazin-1-yl)thiazole-4-carboxylic Acid (18.2)

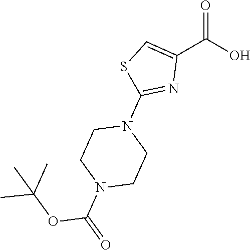 [SMILES: CC(C)(C)OC(=O)N1CCN(c2nc(C(=O)O)cs2)CC1]

Using the procedure described in Example 1.3, ethyl 2-(4-(tert-butoxycarbonyl)piperazin-1-yl)thiazole-4-carboxylate (1.325 g, 3.88 mmole) was converted to 2-(4-(tert-butoxycarbonyl)piperazin-1-yl)thiazole-4-carboxylic acid as a white solid (1.187 g, 98%).

¹H NMR (400 MHz, DMSO-d₆): δ 12.63 (s, 1H), 7.66 (s, 1H), 3.52-3.36 (m, 8H), 1.42 (s, 9H).

### Tert-butyl (S)-4-(4-((3-((tert-butyldimethylsilyl)oxy)-1-methoxy-1-oxopropan-2-yl)carbamoyl)thiazol-2-yl)piperazine-1-carboxylate (18.3)

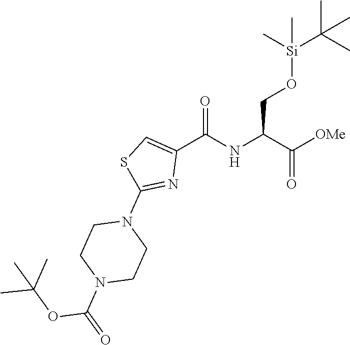 [SMILES: COC(=O)[C@H](CO[Si](C)(C)C(C)(C)C)NC(=O)c1csc(N2CCN(C(=O)OC(C)(C)C)CC2)n1]

Using the procedure described in Example 15.3, 2-(4-(tert-butoxycarbonyl)piperazin-1-yl)thiazole-4-carboxylic acid (1.187 g, 3.79 mmole) was converted to tert-butyl (S)-4-(4-((3-((tert-butyldimethylsilyl)oxy)-1-methoxy-1-oxopropan-2-yl)carbamoyl)thiazol-2-yl)piperazine-1-carboxylate as a white solid (1.920 g, 96%).

¹H NMR (400 MHz, CDCl₃): δ 7.94 (d, J=8.7 Hz, 1H), 7.42 (s, 1H), 4.78 (ddd, J=8.8, 3.4, 2.6 Hz, 1H), 4.19-4.14 (m, 1H), 3.89 (dd, J=10.0, 3.4 Hz, 1H), 3.76 (s, 3H), 3.56 (t, J=5.1 Hz, 4H), 3.48 (q, J=5.3, 4.5 Hz, 4H), 1.49 (s, 9H), 0.88 (s, 9H), 0.05 (s, 3H), 0.03 (s, 3H).

### N-(2-(4-(Tert-butoxycarbonyl)piperazin-1-yl)thiazole-4-carbonyl)-O-(tert-butyldimethylsilyl)-L-serine (18.4)

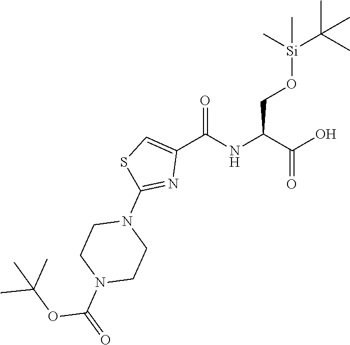 [SMILES: CC(C)(C)OC(=O)N1CCN(c2nc(C(=O)N[C@@H](CO[Si](C)(C)C(C)(C)C)C(=O)O)cs2)CC1]

Using the procedure described in Example 4.3, tert-butyl (S)-4-(4-((3-((tert-butyldimethylsilyl)oxy)-1-methoxy-1-oxopropan-2-yl)carbamoyl)thiazol-2-yl)piperazine-1-carboxylate (1.920 g, 3.63 mmole) was converted to N-(2-(4-(tert-butoxycarbonyl)piperazin-1-yl)thiazole-4-carbonyl)-O-(tert-butyldimethylsilyl)-L-serine as a white solid (1.922 g).

¹H NMR (400 MHz, DMSO-d₆): δ 7.87 (d, J=8.6 Hz, 1H), 7.55 (d, J=6.8 Hz, 1H), 4.53 (dt, J=8.7, 3.3 Hz, 1H), 4.15-4.09 (m, 1H), 3.91 (dd, J=10.3, 3.6 Hz, 1H), 3.52 (s, 8H), 1.47 (s, 9H), 0.89 (s, 9H), 0.08 (s, 3H), 0.06 (s, 3H).

### Tert-butyl 4-(4-(((6S,9S)-9-(methoxycarbonyl)-2,2,3,3-tetramethyl-7,12-dioxo-4,11-dioxa-8-aza-3-silatridecan-6-yl)carbamoyl)thiazol-2-yl)piperazine-1-carboxylate (18.5)

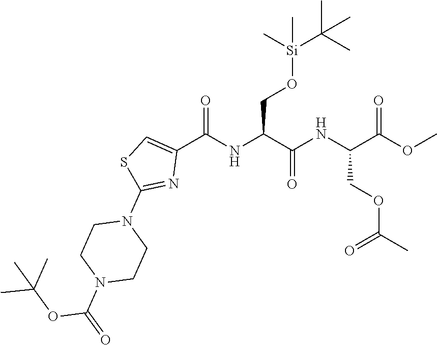 [SMILES: COC(=O)[C@H](COC(C)=O)NC(=O)[C@H](CO[Si](C)(C)C(C)(C)C)NC(=O)c1csc(N2CCN(C(=O)OC(C)(C)C)CC2)n1]

Using the procedure described in Example 15.6, N-(2-(4-(tert-butoxycarbonyl)piperazin-1-yl)thiazole-4-carbonyl)-O-(tert-butyldimethylsilyl)-L-serine (1.922 g, 3.73 mmole) was converted to tert-butyl 4-(4-(((6S,9S)-9-(methoxycarbonyl)-2,2,3,3-tetramethyl-7,12-dioxo-4,11-dioxa-8-aza-3-silatridecan-6-yl)carbamoyl)thiazol-2-yl)piperazine-1-carboxylate as a colorless oil (1.481 g, 60%).

¹H NMR (400 MHz, CDCl₃): δ 7.99 (d, J=7.0 Hz, 1H), 7.48-7.39 (m, 2H), 4.88 (dt, J=7.6, 3.7 Hz, 1H), 4.58 (td, J=7.2, 3.7 Hz, 1H), 4.52-4.45 (m, 1H), 4.32 (dd, J=11.4, 3.6 Hz, 1H), 4.18 (dd, J=9.8, 3.7 Hz, 1H), 3.76 (d, J=2.0 Hz, 3H), 3.73 (d, J=9.7 Hz, 1H), 3.56 (dq, J=5.6, 2.6 Hz, 4H), 3.52-3.43 (m, 4H), 2.05 (s, 3H), 1.49 (s, 9H), 0.93 (s, 9H), 0.14 (s, 3H), 0.13 (s, 3H).

### Tert-butyl 4-(4-((3-((3-methoxy-3-oxoprop-1-en-2-yl)amino)-3-oxoprop-1-en-2-yl)carbamoyl)thiazol-2-yl)piperazine-1-carboxylate (18)

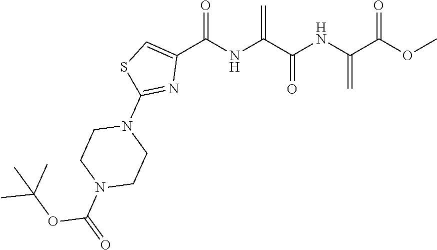 [SMILES: C=C(NC(=O)c1csc(N2CCN(C(=O)OC(C)(C)C)CC2)n1)C(=O)NC(=C)C(=O)OC]

Using the procedure described for Compound 15, tert-butyl 4-(4-(((6S,9S)-9-(methoxycarbonyl)-2,2,3,3-tetramethyl-7,12-dioxo-4,11-dioxa-8-aza-3-silatridecan-6-yl)carbamoyl)thiazol-2-yl)piperazine-1-carboxylate (1.481 g, 2.25 mmole) was converted to tert-butyl 4-(4-((3-((3-methoxy-3-oxoprop-1-en-2-yl)amino)-3-oxoprop-1-en-2-yl)carbamoyl)thiazol-2-yl)piperazine-1-carboxylate as a white solid (0.206 g, 20%).

¹H NMR (400 MHz, CDCl₃): δ 9.73 (s, 1H), 8.52 (s, 1H), 7.46 (s, 1H), 6.72 (d, J=2.1 Hz, 1H), 6.66 (s, 1H), 6.01 (d, J=1.3 Hz, 1H), 5.44 (t, J=1.9 Hz, 1H), 3.89 (s, 3H), 3.58 (dd, J=6.6, 3.5 Hz, 4H), 3.51 (dt, J=7.4, 3.5 Hz, 4H), 1.49 (s, 9H).

### Compound 19: Methyl 2-(2-(2-(4-(6-azidohexanamido)piperidin-1-yl)thiazole-4-carboxamido)acrylamido)acrylate

### Ethyl 2-(4-(6-bromohexanamido)piperidin-1-yl)thiazole-4-carboxylate (19.1)

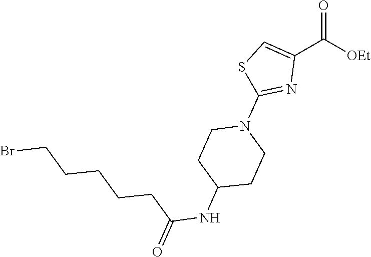 [SMILES: CCOC(=O)c1csc(N2CCC(NC(=O)CCCCCBr)CC2)n1]

6-Bromohexanoic acid (1.212 g, 6.21 mmole) was dissolved in DCM (5.5 mL) and DMF (2 drops) was added. Oxalyl chloride (0.540 mL, 6.19 mmole) was added dropwise (gas evolution was observed) and the solution was stirred at RT for 3 hours, then was concentrated.
In a separate flask, ethyl 2-(4-((tert-butoxycarbonyl)amino)piperidin-1-yl)thiazole-4-carboxylate (1.995 g, 5.61 mmole) was dissolved in DCM (11 mL). HCl (4M solution in 1,4-dioxane, 5.60 mL, 22.4 mmole) was added and the mixture was stirred at RT for 3 hours and was concentrated. The residue was dissolved in DCM (11 mL) and cooled to 0° C. N,N-Diisopropylethylamine (2.00 mL, 11.5 mmole) was added followed by slow addition of a solution of the acid chloride generated above in DCM (2 mL). The resulting mixture was stirred at 0° for 15 minutes, then at RT for 18 hours. Water (50 mL) was added and the two layers were separated. The aqueous layer was extracted with DCM (3×15 mL). The combined organics were dried over magnesium sulfate, filtered, and concentrated. The crude residue was purified by silica gel chromatography (10-40% ethyl acetate/hexane gradient) to provide ethyl 2-(4-(6-bromohexanamido)piperidin-1-yl)thiazole-4-carboxylate as a white solid (1.287 g, 53%).

¹H NMR (400 MHz, CDCl₃): δ 7.44 (s, 1H), 5.34 (d, J=7.9 Hz, 1H), 4.35 (q, J=7.1 Hz, 2H), 4.08-3.97 (m, 3H), 3.41 (t, J=6.7 Hz, 2H), 3.17 (ddd, J=13.3, 11.8, 2.9 Hz, 2H), 2.18 (t, J=7.4 Hz, 2H), 2.10-1.99 (m, 2H), 1.88 (dq, J=7.9, 6.8 Hz, 2H), 1.74-1.62 (m, 2H), 1.57-1.42 (m, 4H), 1.37 (t, J=7.1 Hz, 3H).

### Ethyl 2-(4-(6-azidohexanamido)piperidin-1-yl)thiazole-4-carboxylate (19.2)

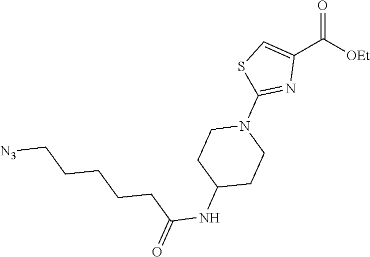 [SMILES: CCOC(=O)c1csc(N2CCC(NC(=O)CCCCCN=[N+]=[N-])CC2)n1]

Ethyl 2-(4-(6-bromohexanamido)piperidin-1-yl)thiazole-4-carboxylate (1.287 g, 2.98 mmole) was dissolved in DMF (6 mL). Sodium azide (0.233 g, 3.58 mmole) was added and the mixture was heated to 80° C. for 18 hours, then was cooled to RT. The mixture was diluted with ethyl acetate (25 mL) and washed with water (3×25 mL). The organics were dried over magnesium sulfate, filtered, and concentrated to provide ethyl 2-(4-(6-azidohexanamido)piperidin-1-yl)thiazole-4-carboxylate as a pale yellow solid (1.127 g, 96%).

¹H NMR (400 MHz, CDCl₃): δ 7.44 (s, 1H), 5.51 (d, J=7.6 Hz, 1H), 4.35 (q, J=7.1 Hz, 2H), 4.01 (dddd, J=11.2, 7.1, 4.4, 2.2 Hz, 3H), 3.28 (t, J=6.8 Hz, 2H), 3.16 (ddd, J=13.4, 11.8, 2.9 Hz, 2H), 2.18 (t, J=7.5 Hz, 2H), 2.04 (d, J=7.3 Hz, 2H), 1.73-1.57 (m, 4H), 1.57-1.46 (m, 2H), 1.46-1.39 (m, 2H), 1.37 (t, J=7.1 Hz, 3H).

### Methyl N-(2-(4-(6-azidohexanamido)piperidin-1-yl)thiazole-4-carbonyl)-O-(tert-butyldimethylsilyl)-L-serinate (19.3)

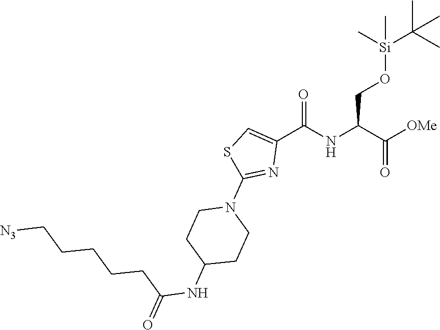 [SMILES: COC(=O)[C@H](CO[Si](C)(C)C(C)(C)C)NC(=O)c1csc(N2CCC(NC(=O)CCCCCN=[N+]=[N-])CC2)n1]

Ethyl 2-(4-(6-azidohexanamido)piperidin-1-yl)thiazole-4-carboxylate (1.127 g, 2.86 mmole) was dissolved in 4/1/1 THF/methanol/water (5.7 mL). Lithium hydroxide monohydrate (0.240 g, 5.72 mmole) was added and the mixture was stirred at RT for 2 hours. Water (40 mL) was added and the solution was treated with 1N aq. HCl to pH=4. The mixture was extracted with ethyl acetate (3×15 mL). The combined organics were dried over magnesium sulfate, filtered, and concentrated. The residue was suspended in DCM (5.7 mL) and L-serine methyl ester hydrochloride (0.535 g, 3.44 mmole) was added. N,N-Diisopropylethylamine (1.00 mL, 5.74 mmole), HOBt·H₂O (0.530 g, 3.46 mmole), and EDC·HCl (0.664 g, 3.46 mmole) were added and the mixture was stirred at RT for 18 hours. Water (25 mL) was added and the mixture was extracted with DCM (3×10 mL). The combined organics were dried over magnesium sulfate, filtered, and concentrated. The residue was dissolved in DCM (5.7 mL). Imidazole (0.215 g, 3.16 mmole) and tert-butyldimethylchlorosilane (0.477 g, 3.16 mmole) were added and the mixture was stirred at RT for 3 hours. Water (25 mL) was added and the mixture was extracted with DCM (3×10 mL). The combined organics were dried over magnesium sulfate, filtered, and concentrated. The crude residue was purified by silica gel chromatography (50-90% ethyl acetate/hexane gradient) to provide methyl N-(2-(4-(6-azidohexanamido)piperidin-1-yl)thiazole-4-carbonyl)-O-(tert-butyldimethylsilyl)-L-serinate as a thick, colorless oil (1.561 g, 94%).

¹H NMR (400 MHz, CDCl₃): δ 7.95 (d, J=8.8 Hz, 1H), 7.38 (s, 1H), 5.44 (d, J=7.9 Hz, 1H), 4.84-4.73 (m, 1H), 4.19-4.14 (m, 1H), 4.07-3.98 (m, 2H), 3.98-3.90 (m, 1H), 3.88 (dd, J=10.0, 3.4 Hz, 1H), 3.76 (s, 3H), 3.28 (t, J=6.8 Hz, 2H), 3.21-3.08 (m, 2H), 2.19 (t, J=7.5 Hz, 2H), 2.03-1.96 (m, 2H), 1.74-1.57 (m, 4H), 1.57-1.47 (m, 2H), 1.47-1.37 (m, 2H), 0.88 (s, 9H), 0.05 (s, 3H), 0.03 (s, 3H).

### Methyl O-acetyl-N—(N-(2-(4-(6-azidohexanamido)piperidin-1-yl)thiazole-4-carbonyl)-O-(tert-butyldimethylsilyl)-L-seryl)-L-serinate (19.4)

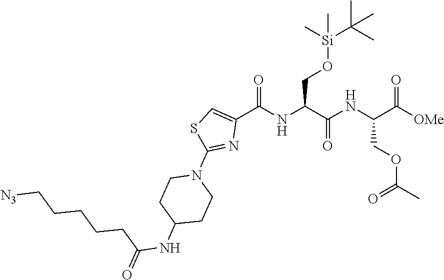 [SMILES: COC(=O)[C@H](COC(C)=O)NC(=O)[C@H](CO[Si](C)(C)C(C)(C)C)NC(=O)c1csc(N2CCC(NC(=O)CCCCCN=[N+]=[N-])CC2)n1]

Using the procedure described in Example 15.6, methyl N-(2-(4-(6-azidohexanamido)piperidin-1-yl)thiazole-4-carbonyl)-O-(tert-butyldimethylsilyl)-L-serinate (1.561 g, 2.68 mmole) was converted to methyl O-acetyl-N—(N-(2-(4-(6-azidohexanamido)piperidin-1-yl)thiazole-4-carbonyl)-O-(tert-butyldimethylsilyl)-L-seryl)-L-serinate as a thick, pale yellow oil (1.312 g, 69%).

¹H NMR (400 MHz, CDCl₃): δ 7.98 (d, J=7.1 Hz, 1H), 7.43 (d, J=7.8 Hz, 1H), 7.40 (d, J=2.6 Hz, 1H), 5.38 (d, J=7.9 Hz, 1H), 4.87 (dq, J=8.0, 4.0 Hz, 1H), 4.58 (td, J=7.2, 3.6 Hz, 1H), 4.52-4.43 (m, 1H), 4.31 (dd, J=11.4, 3.7 Hz, 1H), 4.18 (ddd, J=9.9, 3.7, 1.4 Hz, 1H), 4.08-3.91 (m, 3H), 3.76 (d, J=2.1 Hz, 3H), 3.75-3.69 (m, 1H), 3.28 (t, J=6.8 Hz, 2H), 3.21-3.07 (m, 2H), 2.19 (t, J=7.5 Hz, 2H), 2.01 (s, 3H), 1.73-1.57 (m, 6H), 1.51 (ddd, J=12.6, 9.1, 5.3 Hz, 2H), 1.46-1.38 (m, 2H), 0.92 (s, 9H), 0.14 (s, 3H), 0.12 (s, 3H).

### Methyl 2-(2-(2-(4-(6-azidohexanamido)piperidin-1-yl)thiazole-4-carboxamido)acrylamido)acrylate (19)

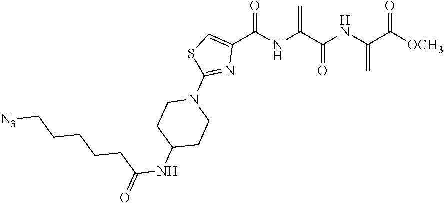 [SMILES: C=C(NC(=O)c1csc(N2CCC(NC(=O)CCCCCN=[N+]=[N-])CC2)n1)C(=O)NC(=C)C(=O)OC]

Using the procedure described for Compound 18, methyl O-acetyl-N—(N-(2-(4-(6-azidohexanamido)piperidin-1-yl)thiazole-4-carbonyl)-O-(tert-butyldimethylsilyl)-L-seryl)-L-serinate (1.312 g, 1.85 mmole) was converted to methyl 2-(2-(2-(4-(6-azidohexanamido)piperidin-1-yl)thiazole-4-carboxamido)acrylamido)acrylate as a white solid (0.481 g, 50%).

¹H NMR (400 MHz, CDCl₃): δ 9.71 (s, 1H), 8.51 (s, 1H), 7.43 (s, 1H), 6.72 (d, J=2.1 Hz, 1H), 6.65 (s, 1H), 6.00 (d, J=1.3 Hz, 1H), 5.44 (t, J=1.9 Hz, 1H), 5.36 (d, J=7.9 Hz, 1H), 4.10-3.96 (m, 3H), 3.89 (s, 3H), 3.28 (t, J=6.8 Hz, 2H), 3.17 (ddd, J=13.3, 11.8, 2.9 Hz, 2H), 2.19 (t, J=7.5 Hz, 2H), 2.11-2.00 (m, 2H), 1.74-1.60 (m, 4H), 1.56-1.46 (m, 2H), 1.46-1.36 (m, 2H).

### Compound 20: Methyl (S)-2-(2-(2-(3-((tert-butoxycarbonyl)amino)piperidin-1-yl)thiazole-4-carboxamido)acrylamido)acrylate

### Ethyl (S)-2-(3-((tert-butoxycarbonyl)amino)piperidin-1-yl)thiazole-4-carboxylate (20.1)

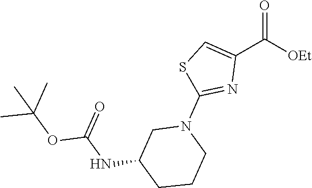 [SMILES: CCOC(=O)c1csc(N2CCC[C@H](NC(=O)OC(C)(C)C)C2)n1]

Ethyl 2-bromothiazole-4-carboxylate (2.003 g, 8.48 mmole) was dissolved in DMA (9 mL). Tert-butyl (S)-piperidin-3-ylcarbamate (1.873 g, 9.35 mmole) was added followed by trimethylamine (1.45 mL, 10.3 mmole), and the mixture was heated to 80° C. for 24 hours, then was cooled to RT and diluted with ethyl acetate (50 mL). The mixture was washed with water (3×25 mL), dried over magnesium sulfate, filtered, and concentrated. The crude residue was purified by silica gel chromatography (30-60% ethyl acetate/hexane gradient) to provide ethyl (S)-2-(3-((tert-butoxycarbonyl)amino)piperidin-1-yl)thiazole-4-carboxylate as a pale yellow solid (2.606 g, 86%).

¹H NMR (400 MHz, CDCl₃): δ 7.44 (s, 1H), 4.71 (s, 1H), 4.35 (q, J=7.1 Hz, 2H), 3.80 (s, 1H), 3.71 (d, J=11.5 Hz, 1H), 3.61 (s, 1H), 3.45 (s, 1H), 3.34-3.20 (m, 1H), 1.91 (ddd, J=12.4, 7.8, 3.6 Hz, 1H), 1.86-1.76 (m, 1H), 1.76-1.64 (m, 1H), 1.60 (s, 1H), 1.51 (s, 9H), 1.37 (t, J=7.1 Hz, 3H).

### Methyl N-(2-((S)-3-((tert-butoxycarbonyl)amino)piperidin-1-yl)thiazole-4-carbonyl)-O-(tert-butyldimethylsilyl)-L-serinate (20.2)

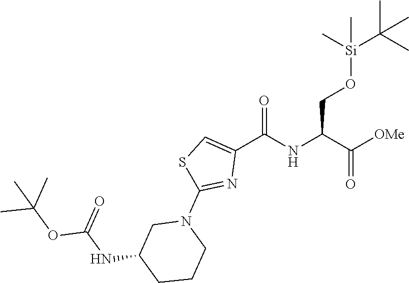 [SMILES: COC(=O)[C@H](CO[Si](C)(C)C(C)(C)C)NC(=O)c1csc(N2CCC[C@H](NC(=O)OC(C)(C)C)C2)n1]

Using the procedure described for Example 22.3, ethyl (S)-2-(3-((tert-butoxycarbonyl)amino)piperidin-1-yl)thiazole-4-carboxylate (0.750 g, 2.11 mmole) was converted to methyl N-(2-((S)-3-((tert-butoxycarbonyl)amino)piperidin-1-yl)thiazole-4-carbonyl)-O-(tert-butyldimethylsilyl)-L-serinate as a colorless oil (1.074 g, 94%).

¹H NMR (400 MHz, CDCl₃): δ 7.94 (d, J=8.8 Hz, 1H), 7.38 (s, 1H), 4.79 (ddd, J=8.8, 3.4, 2.6 Hz, 1H), 4.75-4.65 (m, 1H), 4.19-4.15 (m, 1H), 3.88 (dd, J=10.0, 3.4 Hz, 1H), 3.81 (s, 1H), 3.76 (s, 3H), 3.65 (d, J=12.7 Hz, 1H), 3.55 (d, J=18.5 Hz, 1H), 3.45 (s, 1H), 3.35-3.20 (m, 1H), 1.98-1.76 (m, 2H), 1.71 (dq, J=7.8, 3.8 Hz, 1H), 1.61 (s, 1H), 1.45 (s, 9H), 0.88 (s, 9H), 0.06 (s, 3H), 0.03 (s, 3H).

### Methyl O-acetyl-N—(N-(2-((S)-3-((tert-butoxycarbonyl)amino)piperidin-1-yl)thiazole-4-carbonyl)-O-(tert-butyldimethylsilyl)-L-seryl)-L-serinate (20.3)

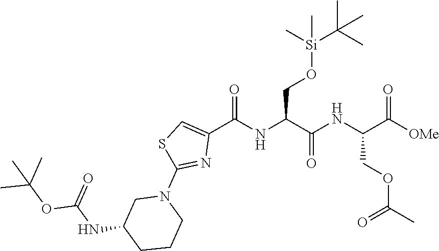 [SMILES: COC(=O)[C@H](COC(C)=O)NC(=O)[C@H](CO[Si](C)(C)C(C)(C)C)NC(=O)c1csc(N2CCC[C@H](NC(=O)OC(C)(C)C)C2)n1]

Using the procedure described in Example 15.6, methyl N-(2-((S)-3-((tert-butoxycarbonyl)amino)piperidin-1-yl)thiazole-4-carbonyl)-O-(tert-butyldimethylsilyl)-L-serinate (1.074 g, 1.98 mmole) was converted to methyl O-acetyl-N—(N-(2-((S)-3-((tert-butoxycarbonyl)amino)piperidin-1-yl)thiazole-4-carbonyl)-O-(tert-butyldimethylsilyl)-L-seryl)-L-serinate as a thick, colorless gel (1.154 g, 87%).

¹H NMR (400 MHz, CDCl₃): δ 7.97 (d, J=7.2 Hz, 1H), 7.43 (d, J=7.7 Hz, 1H), 7.40 (d, J=2.4 Hz, 1H), 4.87 (dt, J=7.7, 3.8 Hz, 1H), 4.69 (s, 1H), 4.59 (td, J=7.1, 3.6 Hz, 1H), 4.52-4.44 (m, 1H), 4.31 (dd, J=11.4, 3.7 Hz, 1H), 4.18 (dd, J=9.8, 3.7 Hz, 1H), 3.76 (d, J=2.8 Hz, 3H), 3.75-3.70 (m, 1H), 3.70-3.52 (m, 2H), 3.44 (s, 1H), 3.36-3.16 (m, 1H), 2.03 (d, J=2.1 Hz, 3H), 1.96-1.86 (m, 1H), 1.87-1.76 (m, 1H), 1.76-1.65 (m, 1H), 1.65-1.59 (m, 1H), 1.45 (s, 9H), 0.92 (s, 9H), 0.14 (s, 3H), 0.13 (s, 3H).

### Methyl (S)-2-(2-(2-(3-((tert-butoxycarbonyl)amino)piperidin-1-yl)thiazole-4-carboxamido)acrylamido)acrylate (20)

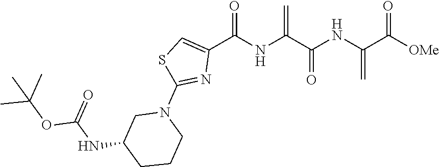 [SMILES: C=C(NC(=O)c1csc(N2CCC[C@H](NC(=O)OC(C)(C)C)C2)n1)C(=O)NC(=C)C(=O)OC]

Using the procedure described for Compound 15, methyl O-acetyl-N—(N-(2-((S)-3-((tert-butoxycarbonyl)amino)piperidin-1-yl)thiazole-4-carbonyl)-O-(tert-butyldimethylsilyl)-L-seryl)-L-serinate (1.154 g, 1.72 mmole) was converted to methyl (S)-2-(2-(2-(3-((tert-butoxycarbonyl)amino)piperidin-1-yl)thiazole-4-carboxamido)acrylamido)acrylate as a white solid (0.372 g, 45%).

¹H NMR (400 MHz, CDCl₃): δ 9.71 (s, 1H), 8.50 (s, 1H), 7.43 (s, 1H), 6.71 (d, J=2.1 Hz, 1H), 6.66 (s, 1H), 6.00 (d, J=1.3 Hz, 1H), 5.43 (t, J=1.8 Hz, 1H), 4.70 (s, 1H), 3.89 (s, 3H), 3.82 (s, 1H), 3.74-3.53 (m, 2H), 3.47 (s, 1H), 3.30 (s, 1H), 1.97-1.78 (m, 2H), 1.72 (qt, J=8.1, 3.7 Hz, 1H), 1.62 (s, 1H), 1.45 (s, 9H).

### Compound 21: Methyl 2-(2-(2-(4-((ethoxycarbonyl)(methyl)amino)piperidin-1-yl)thiazole-4-carboxamido)acrylamido)acrylate

### Tert-butyl 4-((ethoxycarbonyl)(methyl)amino)piperidine-1-carboxylate (21.1)

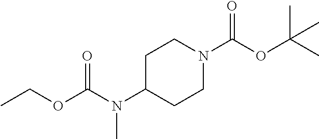 [SMILES: CCOC(=O)N(C)C1CCN(C(=O)OC(C)(C)C)CC1]

Tert-butyl 4-(methylamino)piperidine-1-carboxylate (0.752 g, 3.51 mmole) was dissolved in DCM (7 mL) and cooled to 0° C. N,N-Diisopropylethylamine (0.670 mL, 3.85 mmole) was added followed by dropwise addition of ethyl chloroformate (0.370 mL, 3.87 mmole). The mixture was stirred at 0° for 60 minutes. Water (50 mL) was added and the mixture was extracted with DCM (3×10 mL). The combined organics were dried over magnesium sulfate, filtered, and concentrated. The crude residue was purified by silica gel chromatography (20-60% ethyl acetate/hexane gradient) to provide tert-butyl 4-((ethoxycarbonyl)(methyl)amino)piperidine-1-carboxylate as a colorless oil (0.959 g, 95%).

¹H NMR (400 MHz, CDCl₃): δ 4.27-4.13 (m, 3H), 4.14 (q, J=7.1 Hz, 2H), 2.82-2.69 (m, 2H), 2.76 (s, 3H), 1.60 (qd, J=11.7, 5.8 Hz, 4H), 1.44 (s, 9H), 1.27 (t, J=7.1 Hz, 3H).

### Ethyl 2-(4-((ethoxycarbonyl)(methyl)amino)piperidin-1-yl)thiazole-4-carboxylate (21.2)

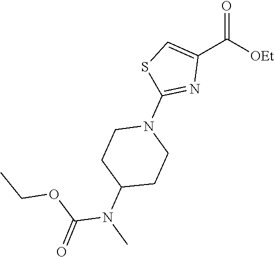 [SMILES: CCOC(=O)c1csc(N2CCC(N(C)C(=O)OCC)CC2)n1]

Tert-butyl 4-((ethoxycarbonyl)(methyl)amino)piperidine-1-carboxylate (0.959 g, 3.35 mmole) was dissolved in DCM (7 mL). HCl (4M solution in 1,4-dioxane, 3.40 mL, 13.6 mmole) was added and the mixture was stirred at RT for 3 hours, then was concentrated. The residue was dissolved in DMA (4 mL). Ethyl 2-bromothiazole-4-carboxylate (0.837 g, 3.55 mmole) was added followed by N,N-diisopropylethylamine (0.940 mL, 5.40 mmole) and the mixture was heated to 80° C. for 18 hours, then was cooled to RT. Ethyl acetate (25 mL) was added and the mixture was washed with water (3×25 mL). The organics were dried over magnesium sulfate, filtered, and concentrated. The crude residue was purified by silica gel chromatography (10-60% ethyl acetate/hexane gradient) to provide ethyl 2-(4-((ethoxycarbonyl)(methyl)amino)piperidin-1-yl)thiazole-4-carboxylate as a yellow oil (0.322 g, 28%).

¹H NMR (400 MHz, CDCl₃): δ 7.44 (s, 1H), 4.35 (q, J=7.1 Hz, 2H), 4.20-4.09 (m, 5H), 3.18-3.05 (m, 2H), 2.77 (s, 3H), 1.78 (qd, J=8.7, 6.8, 3.7 Hz, 4H), 1.37 (t, J=7.1 Hz, 3H), 1.28 (t, J=7.1 Hz, 3H).

### Methyl O-(tert-butyldimethylsilyl)-N-(2-(4-((ethoxycarbonyl)(methyl)amino)piperidin-1-yl)thiazole-4-carbonyl)-L-serinate (21.3)

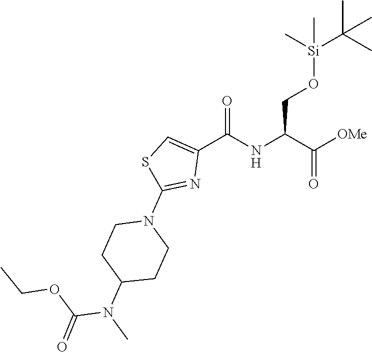 [SMILES: CCOC(=O)N(C)C1CCN(c2nc(C(=O)N[C@@H](CO[Si](C)(C)C(C)(C)C)C(=O)OC)cs2)CC1]

Using the procedure described in Example 19.3, ethyl 2-(4-((ethoxycarbonyl)(methyl)amino)piperidin-1-yl)thiazole-4-carboxylate (0.322 g, 0.943 mmole) was converted to methyl O-(tert-butyldimethylsilyl)-N-(2-(4-((ethoxycarbonyl)(methyl)amino)piperidin-1-yl)thiazole-4-carbonyl)-L-serinate as a thick, colorless oil (0.300 g, 60%).

¹H NMR (400 MHz, CDCl₃): δ 7.95 (d, J=8.8 Hz, 1H), 7.38 (s, 1H), 4.78 (ddd, J=8.8, 3.3, 2.6 Hz, 1H), 4.21-4.02 (m, 6H), 3.89 (dd, J=10.0, 3.4 Hz, 1H), 3.76 (s, 3H), 3.17-3.03 (m, 2H), 2.79 (s, 3H), 1.76 (d, J=4.0 Hz, 4H), 1.33-1.25 (m, 3H), 0.88 (s, 9H), 0.05 (s, 3H), 0.03 (s, 3H).

### Methyl O-acetyl-N—(O-(tert-butyldimethylsilyl)-N-(2-(4-((ethoxycarbonyl)(methyl)amino)piperidin-1-yl)thiazole-4-carbonyl)-L-seryl)-L-serinate (21.4)

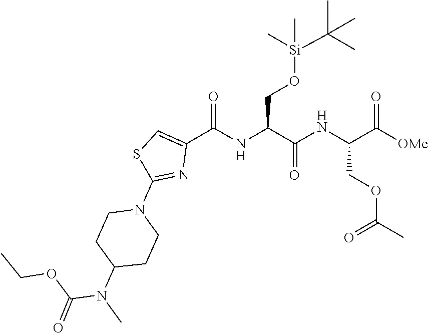 [SMILES: CCOC(=O)N(C)C1CCN(c2nc(C(=O)N[C@@H](CO[Si](C)(C)C(C)(C)C)C(=O)N[C@@H](COC(C)=O)C(=O)OC)cs2)CC1]

Using the procedure described in Example 15.6, methyl O-(tert-butyldimethylsilyl)-N-(2-(4-((ethoxycarbonyl)(methyl)amino)piperidin-1-yl)thiazole-4-carbonyl)-L-serinate (0.300 g, 0.567 mmole) was converted to methyl O-acetyl-N—(O-(tert-butyldimethylsilyl)-N-(2-(4-((ethoxycarbonyl)(methyl)amino)piperidin-1-yl)thiazole-4-carbonyl)-L-seryl)-L-serinate as a white solid (0.297 g, 80%).

¹H NMR (400 MHz, CDCl₃): δ 7.98 (d, J=7.1 Hz, 1H), 7.43 (d, J=7.5 Hz, 1H), 7.39 (d, J=2.5 Hz, 1H), 4.87 (dt, J=7.7, 3.8 Hz, 1H), 4.58 (td, J=7.2, 3.6 Hz, 1H), 4.51-4.45 (m, 1H), 4.31 (dd, J=11.4, 3.7 Hz, 1H), 4.22-4.14 (m, 3H), 4.14-4.04 (m, 3H), 3.76 (d, J=2.2 Hz, 3H), 3.75-3.70 (m, 1H), 3.11 (d, J=12.5 Hz, 2H), 2.78 (s, 3H), 2.02 (d, J=2.7 Hz, 3H), 1.77 (s, 4H), 1.29 (t, J=7.7 Hz, 3H), 0.92 (s, 9H), 0.14 (s, 3H), 0.13 (s, 3H).

### Methyl 2-(2-(2-(4-((ethoxycarbonyl)(methyl)amino)piperidin-1-yl)thiazole-4-carboxamido)acrylamido)acrylate (21)

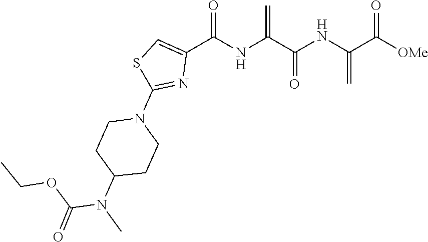 [SMILES: C=C(NC(=O)c1csc(N2CCC(N(C)C(=O)OCC)CC2)n1)C(=O)NC(=C)C(=O)OC]

Using the procedure described for Compound 15, methyl O-acetyl-N—(O-(tert-butyldimethylsilyl)-N-(2-(4-((ethoxycarbonyl)(methyl)amino)piperidin-1-yl)thiazole-4-carbonyl)-L-seryl)-L-serinate (0.297 g, 0.451 mmole) was converted to methyl 2-(2-(2-(4-((ethoxycarbonyl)(methyl)amino)piperidin-1-yl)thiazole-4-carboxamido)acrylamido)acrylate as a white solid (0.090 g, 43%).

¹H NMR (400 MHz, CDCl₃): δ 9.72 (s, 1H), 8.51 (s, 1H), 7.42 (s, 1H), 6.72 (d, J=2.1 Hz, 1H), 6.66 (s, 1H), 6.00 (d, J=1.3 Hz, 1H), 5.44 (t, J=1.8 Hz, 1H), 4.28 (s, 1H), 4.20-4.08 (m, 4H), 3.89 (s, 3H), 3.18-3.04 (m, 2H), 2.79 (s, 3H), 1.79 (s, 4H), 1.28 (t, J=7.1 Hz, 3H).

### Compound 22: Methyl 2-(2-(2-(4-(((ethoxycarbonyl)amino)methyl)piperidin-1-yl)thiazole-4-carboxamido)acrylamido)acrylate

### Tert-butyl 4-(((ethoxycarbonyl)amino)methyl)piperidine-1-carboxylate (22.1)

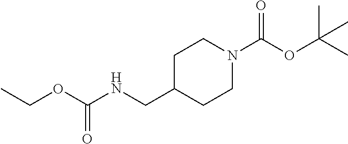 [SMILES: CCOC(=O)NCC1CCN(C(=O)OC(C)(C)C)CC1]

Tert-butyl 4-(aminomethyl)piperidine-1-carboxylate (0.754 g, 3.52 mmole) was dissolved in DCM (7 mL) and cooled to 0° C. N,N-Diisopropylethylamine (0.730 mL, 4.19 mmole) was added followed by dropwise addition of ethyl chloroformate (0.370 mL, 3.87 mmole). The mixture was stirred at 0° for 60 minutes. Water (25 mL) was added and the two layers were separated. The aqueous layer was extracted with DCM (2×10 mL). The combined organics were dried over magnesium sulfate, filtered, and concentrated. The crude residue was purified by silica gel chromatography (20-40% ethyl acetate/hexane gradient) to provide tert-butyl 4-(((ethoxycarbonyl)amino)methyl)piperidine-1-carboxylate as a thick, colorless oil (0.997 g, 99%).

¹H NMR (400 MHz, CDCl₃): δ 4.77 (s, 1H), 4.19-4.03 (m, 4H), 3.07 (t, J=6.4 Hz, 2H), 2.68 (t, J=12.9 Hz, 2H), 1.71-1.63 (m, 2H), 1.44 (s, 9H), 1.25 (dt, J=9.0, 7.1 Hz, 4H), 1.17-1.03 (m, 2H).

### Ethyl 2-(4-(((ethoxycarbonyl)amino)methyl)piperidin-1-yl)thiazole-4-carboxylate (22.2)

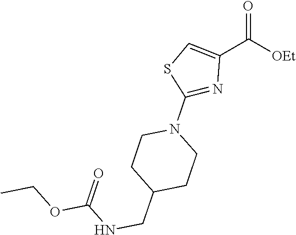 [SMILES: CCOC(=O)NCC1CCN(c2nc(C(=O)OCC)cs2)CC1]

Tert-butyl 4-(((ethoxycarbonyl)amino)methyl)piperidine-1-carboxylate (0.997 g, 3.48 mmole) was dissolved in DCM (7 mL). HCl (4M solution in 1,4-dioxane, 3.50 mL, 14.0 mmole) was added and the mixture was stirred at RT for 3 hours, then was concentrated. The residue was dissolved in DMA (4 mL). Ethyl 2-bromothiazole-4-carboxylate (0.905 g, 3.83 mmole) and N,N-diisopropylethylamine (1.20 mL, 6.89 mmole) were added and the mixture was heated to 80° C. for 18 hours and was cooled to RT, then diluted with ethyl acetate (25 mL). The mixture was washed with water (3×25 mL) and the organics were dried over magnesium sulfate, filtered, and concentrated. The crude residue was purified by silica gel chromatography (10-70% ethyl acetate/hexane gradient) to provide ethyl 2-(4-(((ethoxycarbonyl)amino)methyl)piperidin-1-yl)thiazole-4-carboxylate as a yellow solid (0.600 g, 50%).

¹H NMR (400 MHz, CDCl₃): δ 7.42 (s, 1H), 4.77 (s, 1H), 4.35 (q, J=7.1 Hz, 2H), 4.18-4.02 (m, 4H), 3.11 (t, J=6.4 Hz, 2H), 3.01 (td, J=12.7, 2.8 Hz, 2H), 1.86-1.77 (m, 2H), 1.73 (d, J=6.8 Hz, 1H), 1.37 (t, J=7.1 Hz, 3H), 1.35-1.29 (m, 2H), 1.25 (td, J=7.1, 5.9 Hz, 3H).

### Methyl O-(tert-butyldimethylsilyl)-N-(2-(4-(((ethoxycarbonyl)amino)methyl)piperidin-1-yl)thiazole-4-carbonyl)-L-serinate (22.3)

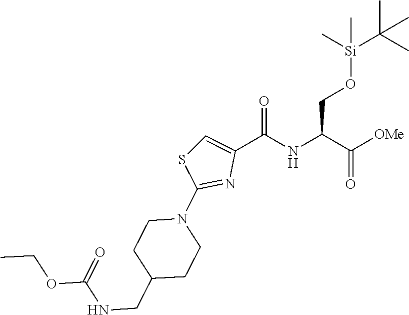 [SMILES: CCOC(=O)NCC1CCN(c2nc(C(=O)N[C@@H](CO[Si](C)(C)C(C)(C)C)C(=O)OC)cs2)CC1]

Using the procedure described in Example 19.3, ethyl 2-(4-(((ethoxycarbonyl)amino)methyl)piperidin-1-yl)thiazole-4-carboxylate (0.600 mL, 1.76 mmole) was converted to methyl O-(tert-butyldimethylsilyl)-N-(2-(4-(((ethoxycarbonyl)amino)methyl)piperidin-1-yl)thiazole-4-carbonyl)-L-serinate as a thick, colorless gel (0.897 g, 96%).

¹H NMR (400 MHz, CDCl₃): δ 7.96 (d, J=8.8 Hz, 1H), 7.36 (s, 1H), 4.78 (dt, J=8.8, 3.0 Hz, 2H), 4.20-4.08 (m, 3H), 4.02 (dd, J=11.7, 5.3 Hz, 2H), 3.88 (dd, J=10.0, 3.4 Hz, 1H), 3.76 (s, 3H), 3.12 (t, J=6.4 Hz, 2H), 2.99 (tdd, J=12.5, 6.0, 2.6 Hz, 2H), 1.81 (s, 2H), 1.34 (tt, J=11.5, 5.2 Hz, 2H), 1.26 (td, J=7.1, 2.9 Hz, 3H), 0.88 (s, 9H), 0.05 (s, 3H), 0.03 (s, 3H).

### Methyl O-acetyl-N—(O-(tert-butyldimethylsilyl)-N-(2-(4-(((ethoxycarbonyl)amino)methyl)piperidin-1-yl)thiazole-4-carbonyl)-L-seryl)-L-serinate (22.4)

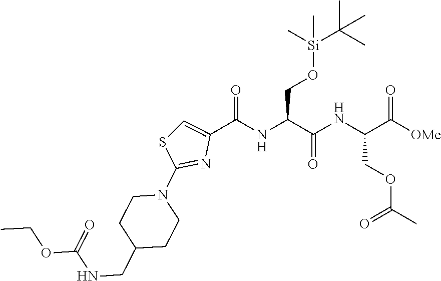 [SMILES: CCOC(=O)NCC1CCN(c2nc(C(=O)N[C@@H](CO[Si](C)(C)C(C)(C)C)C(=O)N[C@@H](COC(C)=O)C(=O)OC)cs2)CC1]

Using the procedure described in Example 15.6, methyl O-(tert-butyldimethylsilyl)-N-(2-(4-(((ethoxycarbonyl)amino)methyl)piperidin-1-yl)thiazole-4-carbonyl)-L-serinate (0.897 g, 1.70 mmole) was converted to methyl O-acetyl-N—(O-(tert-butyldimethylsilyl)-N-(2-(4-(((ethoxycarbonyl)amino)methyl)piperidin-1-yl)thiazole-4-carbonyl)-L-seryl)-L-serinate as a colorless oil (0.921 g, 82%).

¹H NMR (400 MHz, CDCl₃): δ 7.99 (d, J=7.1 Hz, 1H), 7.43 (d, J=7.8 Hz, 1H), 7.37 (d, J=2.8 Hz, 1H), 4.87 (dq, J=8.0, 3.9 Hz, 1H), 4.77 (s, 1H), 4.58 (ddd, J=7.2, 5.9, 3.6 Hz, 1H), 4.47 (dt, J=11.3, 4.2 Hz, 1H), 4.31 (dd, J=11.4, 3.7 Hz, 1H), 4.19 (ddd, J=9.8, 3.6, 2.0 Hz, 1H), 4.12 (q, J=7.1 Hz, 2H), 4.01 (d, J=13.0 Hz, 2H), 3.76 (d, J=2.3 Hz, 3H), 3.75-3.70 (m, 1H), 3.12 (t, J=6.4 Hz, 2H), 2.99 (tt, J=12.8, 3.0 Hz, 2H), 2.02 (d, J=2.4 Hz, 3H), 1.82 (s, 2H), 1.32 (d, J=12.2 Hz, 2H), 1.26 (td, J=7.1, 3.5 Hz, 3H), 0.92 (s, 9H), 0.14 (s, 3H), 0.12 (s, 3H).

### Methyl 2-(2-(2-(4-(((ethoxycarbonyl)amino)methyl)piperidin-1-yl)thiazole-4-carboxamido)acrylamido)acrylate (22)

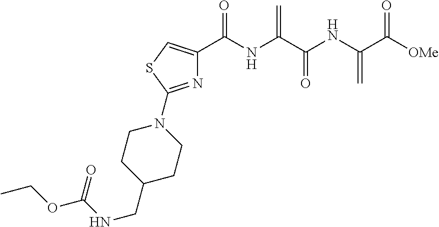 [SMILES: C=C(NC(=O)c1csc(N2CCC(CNC(=O)OCC)CC2)n1)C(=O)NC(=C)C(=O)OC]

Using the procedure as described for Compound 15, methyl O-acetyl-N—(O-(tert-butyldimethylsilyl)-N-(2-(4-(((ethoxycarbonyl)amino)methyl)piperidin-1-yl)thiazole-4-carbonyl)-L-seryl)-L-serinate (0.921 g, 1.40 mmole) was converted to methyl 2-(2-(2-(4-(((ethoxycarbonyl)amino)methyl)piperidin-1-yl)thiazole-4-carboxamido)acrylamido)acrylate as a white solid (0.310 g, 48%).

¹H NMR (400 MHz, CDCl₃): δ 9.78-9.67 (m, 1H), 8.51 (s, 1H), 7.41 (s, 1H), 6.71 (d, J=2.1 Hz, 1H), 6.66 (s, 1H), 6.00 (d, J=1.3 Hz, 1H), 5.43 (t, J=1.9 Hz, 1H), 4.76 (s, 1H), 4.12 (q, J=7.1 Hz, 2H), 4.09-3.98 (m, 2H), 3.89 (s, 3H), 3.12 (t, J=6.4 Hz, 2H), 3.02 (td, J=12.7, 2.8 Hz, 2H), 1.85 (d, J=3.5 Hz, 2H), 1.35 (td, J=12.3, 4.4 Hz, 2H), 1.25 (t, J=7.1 Hz, 3H).

### Compound 23: Methyl (R)-2-(2-(2-(3-((ethoxycarbonyl)amino)pyrrolidin-1-yl)thiazole-4-carboxamido)acrylamido)acrylate

### Tert-butyl (R)-3-((ethoxycarbonyl)amino)pyrrolidine-1-carboxylate (23.1)

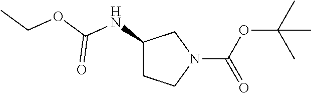 [SMILES: CCOC(=O)N[C@@H]1CCN(C(=O)OC(C)(C)C)C1]

Tert-butyl (R)-3-aminopyrrolidine-1-carboxylate (1.016 g, 5.45 mmole) was dissolved in DCM (11 mL) and cooled to 0° C. N,N-Diisopropylethylamine (1.15 mL, 6.60 mmole) was added followed by ethyl chloroformate (0.560 mL, 5.86 mmole). The mixture was stirred at 0° for 90 minutes and water (50 mL) was added. The two layers were separated and the aqueous layer was extracted with DCM (2×15 mL). The combined organics were dried over magnesium sulfate, filtered, and concentrated. The crude residue was purified by silica gel chromatography (30-60% ethyl acetate/hexane) to provide tert-butyl (R)-3-((ethoxycarbonyl)amino)pyrrolidine-1-carboxylate as a colorless oil (1.597 g).

¹H NMR (400 MHz, CDCl₃): δ 4.84 (s, 1H), 4.23 (d, J=8.2 Hz, 1H), 4.12 (q, J=7.1 Hz, 2H), 3.60 (dd, J=11.4, 6.2 Hz, 1H), 3.50-3.34 (m, 2H), 3.20 (d, J=25.0 Hz, 1H), 2.13 (dtd, J=13.2, 7.5, 5.9 Hz, 1H), 1.90-1.75 (m, 1H), 1.43 (s, 9H), 1.25 (td, J=7.2, 5.6 Hz, 3H).

### Ethyl (R)-2-(3-((ethoxycarbonyl)amino)pyrrolidin-1-yl)thiazole-4-carboxylate (23.2)

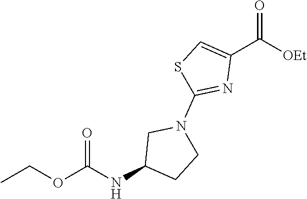 [SMILES: CCOC(=O)N[C@@H]1CCN(c2nc(C(=O)OCC)cs2)C1]

Tert-butyl (R)-3-((ethoxycarbonyl)amino)pyrrolidine-1-carboxylate (1.597 g, 6.18 mmole) was dissolved in DCM (12 mL). HCl (4M solution in 1,4-dioxane, 6.20 mL, 24.8 mmole) was added and the mixture was stirred at RT for 75 minutes and was concentrated. The residue was dissolved in DMA (6 mL) and ethyl 2-bromothiazole-4-carboxylate (1.613 g, 6.83 mmole) and N,N-diisopropylethylamine (2.15 mL, 12.3 mmole) were added. The mixture was heated to 80° C. for 3 days and was cooled to RT. Ethyl acetate (25 mL) was added and the mixture was washed with water (3×25 mL). The organics were dried over magnesium sulfate, filtered, and concentrated. The residue was purified by silica gel chromatography (30-70% ethyl acetate/hexane gradient) to provide ethyl (R)-2-(3-((ethoxycarbonyl)amino)pyrrolidin-1-yl)thiazole-4-carboxylate as a white solid (1.105 g, 57%).

¹H NMR (400 MHz, CDCl₃): δ 7.40 (s, 1H), 4.83 (s, 1H), 4.47-4.39 (m, 1H), 4.36 (q, J=7.1 Hz, 2H), 4.13 (q, J=7.0 Hz, 2H), 3.79 (dd, J=10.7, 6.0 Hz, 1H), 3.61 (qt, J=10.0, 6.4 Hz, 2H), 3.43 (dd, J=10.6, 4.2 Hz, 1H), 2.33 (dddd, J=13.0, 8.1, 7.2, 6.0 Hz, 1H), 2.09-1.96 (m, 1H), 1.37 (t, J=7.1 Hz, 3H), 1.25 (t, J=7.1 Hz, 3H).

### Methyl O-(tert-butyldimethylsilyl)-N-(2-((R)-3-((ethoxycarbonyl)amino)pyrrolidin-1-yl)thiazole-4-carbonyl)-L-serinate (23.3)

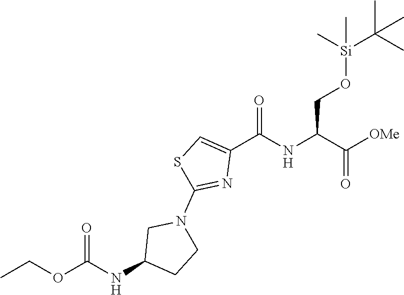 [SMILES: CCOC(=O)N[C@@H]1CCN(c2nc(C(=O)N[C@@H](CO[Si](C)(C)C(C)(C)C)C(=O)OC)cs2)C1]

Using the procedure described in Example 19.3, ethyl (R)-2-(3-((ethoxycarbonyl)amino)pyrrolidin-1-yl)thiazole-4-carboxylate (1.105 g, 3.53 mmole) was converted to methyl O-(tert-butyldimethylsilyl)-N-(2-((R)-3-((ethoxycarbonyl)amino)pyrrolidin-1-yl)thiazole-4-carbonyl)-L-serinate as a colorless oil (0.859 g, 49%).

¹H NMR (400 MHz, CDCl₃): δ 7.99 (d, J=8.8 Hz, 1H), 7.34 (s, 1H), 4.90 (s, 1H), 4.84-4.75 (m, 1H), 4.43 (s, 1H), 4.19-4.12 (m, 3H), 3.89 (dd, J=10.0, 3.4 Hz, 1H), 3.76 (s, 3H), 3.75-3.69 (m, 1H), 3.59-3.51 (m, 2H), 3.38 (dd, J=10.8, 4.2 Hz, 1H), 2.41-2.27 (m, 1H), 2.07-1.98 (m, 1H), 1.26 (t, J=7.1 Hz, 3H), 0.89 (s, 9H), 0.06 (s, 3H), 0.03 (s, 3H).

### Methyl O-acetyl-N—(O-(tert-butyldimethylsilyl)-N-(2-((R)-3-((ethoxycarbonyl)amino)pyrrolidin-1-yl)thiazole-4-carbonyl)-L-seryl)-L-serinate (23.4)

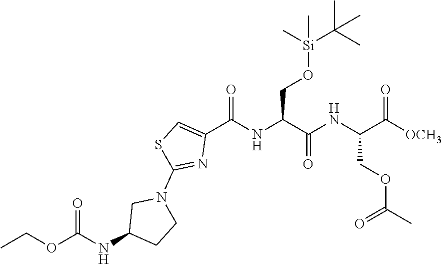 [SMILES: CCOC(=O)N[C@@H]1CCN(c2nc(C(=O)N[C@@H](CO[Si](C)(C)C(C)(C)C)C(=O)N[C@@H](COC(C)=O)C(=O)OC)cs2)C1]

Using the procedure described in Example 15.6, methyl O-(tert-butyldimethylsilyl)-N-(2-((R)-3-((ethoxycarbonyl)amino)pyrrolidin-1-yl)thiazole-4-carbonyl)-L-serinate (0.859 g, 1.72 mmole) was converted to methyl O-acetyl-N—(O-(tert-butyldimethylsilyl)-N-(2-((R)-3-((ethoxycarbonyl)amino)pyrrolidin-1-yl)thiazole-4-carbonyl)-L-seryl)-L-serinate as a pale yellow oil (0.753 g, 70%).

¹H NMR (400 MHz, CDCl₃): δ 8.02 (d, J=7.2 Hz, 1H), 7.44 (d, J=7.8 Hz, 1H), 7.35 (d, J=2.4 Hz, 1H), 4.87 (dq, J=7.7, 3.9 Hz, 2H), 4.59 (ddd, J=7.2, 6.0, 3.7 Hz, 1H), 4.51-4.44 (m, 1H), 4.43 (d, J=7.5 Hz, 1H), 4.31 (dd, J=11.4, 3.7 Hz, 1H), 4.18 (dd, J=9.8, 3.7 Hz, 1H), 4.12 (q, J=7.2 Hz, 3H), 3.76 (d, J=2.5 Hz, 4H), 3.75-3.70 (m, 1H), 3.56 (ddt, J=7.8, 5.8, 2.7 Hz, 2H), 3.38 (dd, J=10.7, 4.2 Hz, 1H), 2.41-2.27 (m, 1H), 2.06-1.99 (m, 1H), 2.02 (s, 3H), 1.26 (td, J=7.1, 2.2 Hz, 3H), 0.93 (d, J=1.4 Hz, 9H), 0.14 (s, 3H), 0.13 (s, 3H).

### Methyl (R)-2-(2-(2-(3-((ethoxycarbonyl)amino)pyrrolidin-1-yl)thiazole-4-carboxamido)acrylamido)acrylate (23)

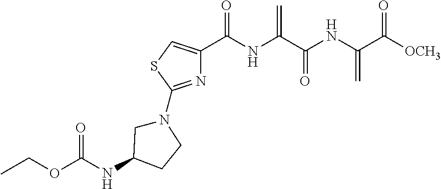 [SMILES: C=C(NC(=O)c1csc(N2CC[C@@H](NC(=O)OCC)C2)n1)C(=O)NC(=C)C(=O)OC]

Using the procedure as described for Compound 15, methyl O-acetyl-N—(O-(tert-butyldimethylsilyl)-N-(2-((R)-3-((ethoxycarbonyl)amino)pyrrolidin-1-yl)thiazole-4-carbonyl)-L-seryl)-L-serinate (0.753 g, 1.20 mmole) was converted to methyl (R)-2-(2-(2-(3-((ethoxycarbonyl)amino)pyrrolidin-1-yl)thiazole-4-carboxamido)acrylamido)acrylate as a white solid (0.224 g, 43%).

¹H NMR (400 MHz, CDCl₃): δ 9.77 (s, 1H), 8.50 (s, 1H), 7.38 (s, 1H), 6.71 (d, J=2.1 Hz, 1H), 6.67 (s, 1H), 6.00 (d, J=1.3 Hz, 1H), 5.43 (t, J=1.9 Hz, 1H), 4.85 (s, 1H), 4.44 (s, 1H), 4.13 (p, J=7.0 Hz, 2H), 3.89 (s, 3H), 3.79 (dd, J=10.7, 6.1 Hz, 1H), 3.69-3.52 (m, 2H), 3.41 (dd, J=10.6, 4.3 Hz, 1H), 2.35 (dddd, J=12.9, 8.1, 7.1, 6.0 Hz, 1H), 2.04 (d, J=5.0 Hz, 1H), 1.32-1.20 (m, 3H).

### Compound 24: Methyl 2-(2-(2-(4-(2-methoxyethoxy)phenyl)thiazole-4-carboxamido)acrylamido)acrylate

### 4-(2-Methoxyethoxy)benzonitrile (24.1)

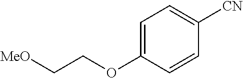 [SMILES: COCCOc1ccc(C#N)cc1]

2-Methoxyethanol (1.008 g, 13.2 mmole) was dissolved in DMF (13 mL). Sodium hydride (60% dispersion in mineral oil, 0.578 g, 14.5 mmole) was added portionwise (gas evolution was observed). The mixture was stirred at RT for 30 minutes and 4-fluorobenzonitrile (1.670 g, 13.8 mmole) was added. The mixture was heated to 80° C. for 2½ hours and was cooled to RT. Ethyl acetate (25 mL) was added and the mixture was washed with water (3×25 mL). The organics were dried over magnesium sulfate, filtered, and concentrated. The crude residue was purified by silica gel chromatography (5-55% ethyl acetate/hexane gradient) to provide 4-(2-methoxyethoxy)benzonitrile as a colorless solid (1.406 g, 60%).

¹H NMR (400 MHz, CDCl₃): δ 7.62-7.55 (m, 2H), 7.02-6.94 (m, 2H), 4.20-4.13 (m, 2H), 3.80-3.73 (m, 2H), 3.45 (s, 3H).

### 4-(2-Methoxyethoxy)benzothioamide (24.2)

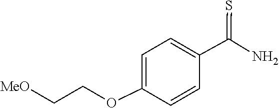 [SMILES: COCCOc1ccc(C(N)=S)cc1]

4-(2-Methoxyethoxy)benzonitrile (1.406 g, 7.93 mmole) was dissolved in pyridine (8 mL). Triethylamine (1.20 mL, 8.56 mmole) was added followed by ammonium sulfide (40% aqueous solution, 1.60 mL, 9.36 mmole). The mixture was heated to 50° C. for 20 hours and was cooled to RT, then was concentrated. The residue was treated with diethyl ether (20 mL) and the solid was collected by filtration to provide 4-(2-methoxyethoxy)benzothioamide as a yellow solid (1.070 g, 64%).

¹H NMR (400 MHz, DMSO-d₆): δ 9.64 (s, 1H), 9.32 (s, 1H), 8.01-7.89 (m, 2H), 7.01-6.91 (m, 2H), 4.21-4.11 (m, 2H), 3.72-3.62 (m, 2H), 3.31 (s, 3H).

### Ethyl 2-(4-(2-methoxyethoxy)phenyl)thiazole-4-carboxylate (24.3)

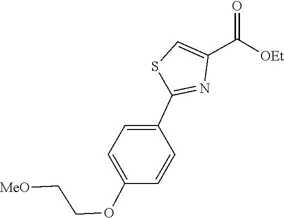 [SMILES: CCOC(=O)c1csc(-c2ccc(OCCOC)cc2)n1]

4-(2-Methoxyethoxy)benzothioamide (1.070 g, 5.06 mmole) was dissolved in ethanol (10 mL). Ethyl bromopyruvate (0.770 mL, 6.14 mmole) was added and the mixture was heated to 80° C. for 3 hours, then was cooled to RT, causing a solid to precipitate from solution. The solid was collected by filtration, washed with cold ethanol, and allowed to air dry to provide ethyl 2-(4-(2-methoxyethoxy)phenyl)thiazole-4-carboxylate as a yellow solid (0.931 g, 60%).

¹H NMR (400 MHz, CDCl₃): δ 8.09 (s, 1H), 7.98-7.90 (m, 2H), 7.02-6.94 (m, 2H), 4.44 (q, J=7.1 Hz, 2H), 4.22-4.14 (m, 2H), 3.82-3.74 (m, 2H), 3.46 (s, 3H), 1.43 (t, J=7.1 Hz, 3H).

### Methyl O-(tert-butyldimethylsilyl)-N-(2-(4-(2-methoxyethoxy)phenyl)thiazole-4-carbonyl)-L-serinate (24.4)

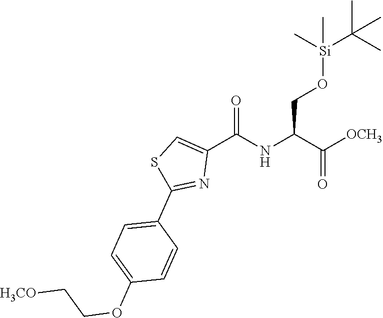 [SMILES: COCCOc1ccc(-c2nc(C(=O)N[C@@H](CO[Si](C)(C)C(C)(C)C)C(=O)OC)cs2)cc1]

Using the procedure described in Example 19.3, ethyl 2-(4-(2-methoxyethoxy)phenyl)thiazole-4-carboxylate (0.931 g, 3.03 mmole) was converted to methyl O-(tert-butyldimethylsilyl)-N-(2-(4-(2-methoxyethoxy)phenyl)thiazole-4-carbonyl)-L-serinate as a colorless oil (1.481 g, 99%).

¹H NMR (400 MHz, CDCl₃): δ 8.23 (d, J=8.7 Hz, 1H), 8.03 (s, 1H), 7.94-7.85 (m, 2H), 7.04-6.95 (m, 2H), 4.85 (dt, J=8.7, 3.1 Hz, 1H), 4.24-4.16 (m, 3H), 3.95 (dd, J=10.0, 3.4 Hz, 1H), 3.83-3.74 (m, 2H), 3.74 (s, 3H), 3.47 (s, 3H), 0.92 (s, 9H), 0.07 (s, 3H), 0.06 (s, 3H).

### Methyl O-acetyl-N—(O-(tert-butyldimethylsilyl)-N-(2-(4-(2-methoxyethoxy)phenyl)thiazole-4-carbonyl)-L-seryl)-L-serinate (24.5)

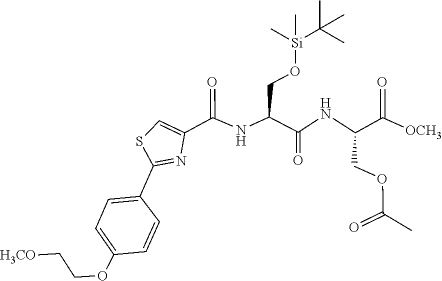 [SMILES: COCCOc1ccc(-c2nc(C(=O)N[C@@H](CO[Si](C)(C)C(C)(C)C)C(=O)N[C@@H](COC(C)=O)C(=O)OC)cs2)cc1]

Using the procedure described in Example 15.6, methyl O-(tert-butyldimethylsilyl)-N-(2-(4-(2-methoxyethoxy)phenyl)thiazole-4-carbonyl)-L-serinate (0.701 g, 1.42 mmole) was converted to methyl O-acetyl-N—(O-(tert-butyldimethylsilyl)-N-(2-(4-(2-methoxyethoxy)phenyl)thiazole-4-carbonyl)-L-seryl)-L-serinate as a colorless oil (0.644 g, 73%).

¹H NMR (400 MHz, CDCl₃): δ 8.27 (d, J=7.3 Hz, 1H), 8.04 (d, J=2.8 Hz, 1H), 7.95-7.85 (m, 2H), 7.46 (d, J=7.8 Hz, 1H), 6.99 (dd, J=8.3, 1.5 Hz, 2H), 4.89 (tt, J=6.5, 3.8 Hz, 1H), 4.66 (ddd, J=8.9, 6.5, 3.7 Hz, 1H), 4.54-4.46 (m, 1H), 4.33 (dd, J=11.4, 3.6 Hz, 1H), 4.23 (dd, J=9.8, 3.7 Hz, 1H), 4.21-4.17 (m, 2H), 3.82-3.78 (m, 2H), 3.78-3.74 (m, 1H), 3.76 (s, 3H), 3.47 (s, 3H), 2.01 (s, 3H), 0.95 (s, 9H), 0.15 (s, 3H), 0.14 (s, 3H).

### Methyl 2-(2-(2-(4-(2-methoxyethoxy)phenyl)thiazole-4-carboxamido)acrylamido)acrylate (24)

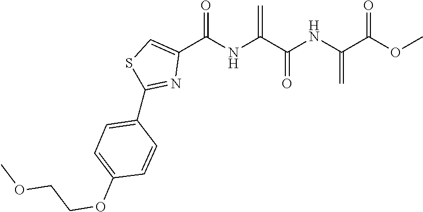

Using the procedure as described for Compound 15, methyl O-acetyl-N—(O-(tert-butyldimethylsilyl)-N-(2-(4-(2-methoxyethoxy)phenyl)thiazole-4-carbonyl)-L-seryl)-L-serinate (0.644 g, 1.03 mmole) was converted to methyl 2-(2-(2-(4-(2-methoxyethoxy)phenyl)thiazole-4-carboxamido)acrylamido)acrylate as a white solid (0.203 g, 46%).

¹H NMR (400 MHz, CDCl₃): δ 10.01 (s, 1H), 8.55 (s, 1H), 8.08 (s, 1H), 7.99-7.90 (m, 2H), 7.06-6.96 (m, 2H), 6.78 (d, J=2.2 Hz, 1H), 6.71 (s, 1H), 6.03 (d, J=1.3 Hz, 1H), 5.49 (t, J=1.9 Hz, 1H), 4.25-4.13 (m, 2H), 3.90 (s, 3H), 3.85-3.74 (m, 2H), 3.48 (s, 3H).

### Compound 25: Methyl 2-(2-(2-(4-(6-azidohexanamido)phenyl)thiazole-4-carboxamido)acrylamido)acrylate

### Tert-butyl (4-carbamothioylphenyl)carbamate (25.1)

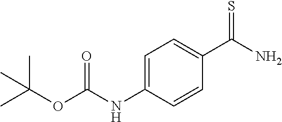 [SMILES: CC(C)(C)OC(=O)Nc1ccc(C(N)=S)cc1]

Tert-butyl (4-cyanophenyl)carbamate (1.002 g, 4.59 mmole) was dissolved in pyridine (5 mL). Triethylamine (0.710 mL, 5.07 mmole) and ammonium sulfide (40% aqueous solution, 0.940 mL, 5.50 mmole) were added and the mixture was heated to 50° C. for 18 hours, then was cooled to RT and concentrated. The residue was suspended in ethyl acetate (25 mL) and the mixture was washed with 1N aq. HCl (2×25 mL). The organics were dried over magnesium sulfate, filtered, and concentrated to provide tert-butyl (4-carbamothioylphenyl)carbamate as a yellow solid (1.102 g, 95%).

¹H NMR (400 MHz, DMSO-d₆): δ 9.64 (d, J=4.7 Hz, 2H), 9.30 (s, 1H), 7.92-7.83 (m, 2H), 7.52-7.42 (m, 2H), 1.48 (s, 9H).

### Ethyl 2-(4-(6-bromohexanamido)phenyl)thiazole-4-carboxylate (25.2)

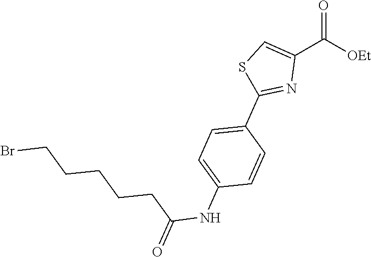 [SMILES: CCOC(=O)c1csc(-c2ccc(NC(=O)CCCCCBr)cc2)n1]

Tert-butyl (4-carbamothioylphenyl)carbamate (1.102 g, 4.37 mmole) was dissolved in ethanol (9 mL). Ethyl bromopyruvate (0.610 mL, 4.86 mmole) was added and the mixture was heated to 80° C. for 2 hours then was cooled to RT and concentrated to provide ethyl 2-(4-aminophenyl)thiazole-4-carboxylate hydrobromide as an orange solid (1.591 g). 6-Bromohexanoic acid (1.373 g, 7.04 mmole) was dissolved in DCM (13 mL). DMF (2 drops) was added followed by slow addition of oxalyl chloride (0.620 mL, 7.11 mmole; gas evolution was observed). The solution was stirred at RT for 2 hours and was concentrated. The residue was dissolved in DCM (2 mL) and added slowly to a cold (0° C.) mixture of ethyl 2-(4-aminophenyl)thiazole-4-carboxylate hydrobromide (1.591 g) and N,N-diisopropylethylamine (2.25 mL, 12.9 mmole) in DCM (13 mL). The resulting mixture was stirred at 0° for 90 minutes and was poured into 1N aq. HCl (25 mL) and water (25 mL). The two layers were separated and the aqueous layer was extracted with DCM (2×15 mL). The combined organics were dried over magnesium sulfate, filtered, and concentrated. The crude residue was purified by silica gel chromatography (10-70% ethyl acetate/hexane gradient) to provide ethyl 2-(4-(6-bromohexanamido)phenyl)thiazole-4-carboxylate as a yellow solid (1.211 g, 65%).

¹H NMR (400 MHz, CDCl₃): δ 8.12 (s, 1H), 8.00-7.91 (m, 2H), 7.68 (d, J=8.7 Hz, 2H), 7.64 (s, 1H), 4.45 (q, J=7.1 Hz, 2H), 3.42 (t, J=6.7 Hz, 2H), 2.42 (t, J=7.4 Hz, 2H), 1.90 (dq, J=10.4, 6.8 Hz, 2H), 1.77 (tt, J=8.1, 6.3 Hz, 2H), 1.53 (ddt, J=12.2, 6.5, 3.9 Hz, 2H), 1.43 (t, J=7.1 Hz, 3H).

### Ethyl 2-(4-(6-azidohexanamido)phenyl)thiazole-4-carboxylate (25.3)

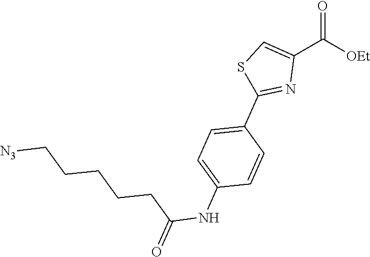 [SMILES: CCOC(=O)c1csc(-c2ccc(NC(=O)CCCCCN=[N+]=[N-])cc2)n1]

Ethyl 2-(4-(6-bromohexanamido)phenyl)thiazole-4-carboxylate (1.211 g, 2.85 mmole) was dissolved in DMF (6 mL). Sodium azide (0.224 g, 3.45 mmole) was added and the mixture was heated to 80° C. for 3 hours. The mixture was cooled to RT and diluted with ethyl acetate (25 mL). The mixture was washed with water (3×25 mL) and sat. aq. sodium chloride (1×25 mL), then dried over magnesium sulfate, filtered, and concentrated to provide ethyl 2-(4-(6-azidohexanamido)phenyl)thiazole-4-carboxylate as a yellow solid (0.899 g, 81%).

¹H NMR (400 MHz, CDCl₃): δ 8.12 (s, 1H), 8.00-7.93 (m, 2H), 7.66 (d, J=8.6 Hz, 2H), 7.55 (s, 1H), 4.45 (q, J=7.1 Hz, 2H), 3.29 (t, J=6.8 Hz, 2H), 2.41 (t, J=7.4 Hz, 2H), 1.77 (tt, J=8.1, 6.4 Hz, 2H), 1.71-1.58 (m, 2H), 1.51-1.46 (m, 2H), 1.43 (t, J=7.1 Hz, 3H).

### Methyl N-(2-(4-(6-azidohexanamido)phenyl)thiazole-4-carbonyl)-O-(tert-butyldimethylsilyl)-L-serinate (25.4)

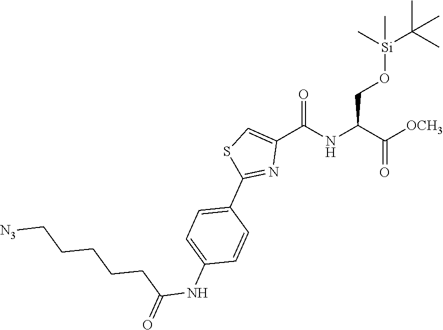 [SMILES: COC(=O)[C@H](CO[Si](C)(C)C(C)(C)C)NC(=O)c1csc(-c2ccc(NC(=O)CCCCCN=[N+]=[N-])cc2)n1]

Using the procedure described for Example 19.3, ethyl 2-(4-(6-azidohexanamido)phenyl)thiazole-4-carboxylate (0.899 g, 2.32 mmole) was converted to methyl N-(2-(4-(6-azidohexanamido)phenyl)thiazole-4-carbonyl)-O-(tert-butyldimethylsilyl)-L-serinate as a thick, orange oil (0.615 g, 46%).

¹H NMR (400 MHz, CDCl₃): δ 8.22 (d, J=8.7 Hz, 1H), 8.05 (s, 1H), 7.94-7.85 (m, 2H), 7.62 (d, J=8.6 Hz, 2H), 7.50 (s, 1H), 4.85 (dt, J=8.7, 3.1 Hz, 1H), 4.22 (dd, J=10.1, 2.7 Hz, 1H), 3.95 (dd, J=10.1, 3.4 Hz, 1H), 3.79 (s, 3H), 3.30 (t, J=6.8 Hz, 2H), 2.41 (t, J=7.4 Hz, 2H), 1.84-1.72 (m, 2H), 1.72-1.59 (m, 2H), 1.54-1.43 (m, 2H), 0.92 (s, 9H), 0.08 (s, 3H), 0.06 (s, 3H).

### Methyl O-acetyl-N—(N-(2-(4-(6-azidohexanamido)phenyl)thiazole-4-carbonyl)-O-(tert-butyldimethylsilyl)-L-seryl)-L-serinate (25.5)

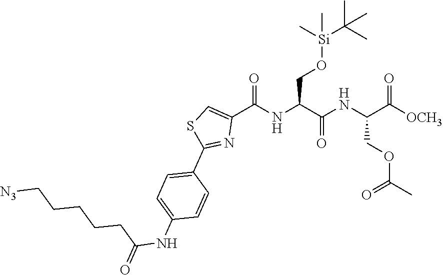 [SMILES: COC(=O)[C@H](COC(C)=O)NC(=O)[C@H](CO[Si](C)(C)C(C)(C)C)NC(=O)c1csc(-c2ccc(NC(=O)CCCCCN=[N+]=[N-])cc2)n1]

Using the procedure described in Example 15.6, methyl N-(2-(4-(6-azidohexanamido)phenyl)thiazole-4-carbonyl)-O-(tert-butyldimethylsilyl)-L-serinate (0.615 g, 1.07 mmole) was converted to methyl O-acetyl-N—(N-(2-(4-(6-azidohexanamido)phenyl)thiazole-4-carbonyl)-O-(tert-butyldimethylsilyl)-L-seryl)-L-serinate as a pale yellow oil (0.517 g, 69%).

¹H NMR (400 MHz, CDCl₃): δ 8.25 (dd, J=7.3, 4.4 Hz, 1H), 8.07 (d, J=3.4 Hz, 1H), 7.96-7.88 (m, 2H), 7.63 (d, J=8.3 Hz, 2H), 7.47 (d, J=7.7 Hz, 1H), 7.42 (d, J=7.6 Hz, 1H), 4.95-4.85 (m, 1H), 4.67 (td, J=7.2, 3.7 Hz, 1H), 4.55-4.45 (m, 1H), 4.34 (dd, J=11.4, 3.6 Hz, 1H), 4.22 (dt, J=9.9, 3.8 Hz, 1H), 3.81 (dd, J=9.8, 7.4 Hz, 1H), 3.77 (s, 3H), 3.31 (t, J=6.8 Hz, 2H), 2.42 (t, J=7.4 Hz, 2H), 2.01 (s, 3H), 1.85-1.74 (m, 2H), 1.71-1.64 (m, 2H), 1.56-1.43 (m, 2H), 0.96 (s, 9H), 0.16 (s, 3H), 0.15 (s, 3H).

### Methyl 2-(2-(2-(4-(6-azidohexanamido)phenyl)thiazole-4-carboxamido)acrylamido)acrylate (25)

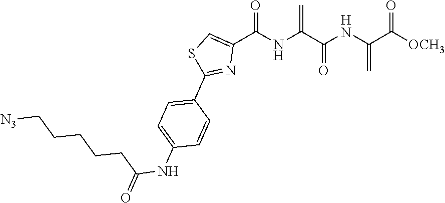 [SMILES: C=C(NC(=O)c1csc(-c2ccc(NC(=O)CCCCCN=[N+]=[N-])cc2)n1)C(=O)NC(=C)C(=O)OC]

Using the procedure as described for Compound 15, methyl O-acetyl-N—(N-(2-(4-(6-azidohexanamido)phenyl)thiazole-4-carbonyl)-O-(tert-butyldimethylsilyl)-L-seryl)-L-serinate (0.517 g, 0.734 mmole) converted to methyl 2-(2-(2-(4-(6-azidohexanamido)phenyl)thiazole-4-carboxamido)acrylamido)acrylate as a white solid (0.093 g, 25%).

¹H NMR (400 MHz, CDCl₃): δ 10.00 (s, 1H), 8.55 (s, 1H), 8.12 (s, 1H), 8.03-7.92 (m, 2H), 7.64 (d, J=8.3 Hz, 2H), 7.29 (s, 1H), 6.78 (d, J=2.2 Hz, 1H), 6.71 (s, 1H), 6.04 (d, J=1.3 Hz, 1H), 5.50 (t, J=1.9 Hz, 1H), 3.91 (s, 3H), 3.31 (t, J=6.8 Hz, 2H), 2.42 (t, J=7.4 Hz, 2H), 1.80 (tt, J=9.5, 6.5 Hz, 2H), 1.73-1.60 (m, 2H), 1.54-1.43 (m, 2H).

### Compound 26: Methyl 2-(2-(2-(4-((((2-methoxyethoxy)carbonyl)amino)methyl)phenyl)thiazole-4-carboxamido)acrylamido)acrylate

### 2-Methoxyethyl 1H-imidazole-1-carboxylate (26.1)

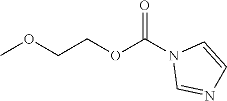

Following a literature procedure (RSC Advances, 2014, 4(25), 13012-13017) (27), 2-methoxyethanol (1.05 mL, 13.3 mmole) was dissolved in THF (26 mL). N,N′-Carbonyldiimidazole (2.560 g, 15.8 mmole) was added and the solution was stirred at RT for 20 hours. Water (100 mL) was added and the mixture was extracted with ethyl acetate (3×25 mL). The combined organics were dried over magnesium sulfate, filtered, and concentrated to provide 2-methoxyethyl 1H-imidazole-1-carboxylate as a colorless oil (2.757 g).

¹H NMR (400 MHz, CDCl₃): δ 8.20-8.12 (m, 1H), 7.45 (t, J=1.5 Hz, 1H), 7.07 (dd, J=1.7, 0.9 Hz, 1H), 4.60-4.52 (m, 2H), 3.76-3.69 (m, 2H), 3.42 (s, 3H).

### 2-Methoxyethyl (4-cyanobenzyl)carbamate (26.2)

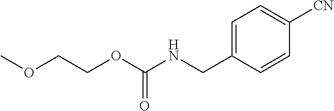 [SMILES: COCCOC(=O)NCc1ccc(C#N)cc1]

2-Methoxyethyl 1H-imidazole-1-carboxylate (1.261 g, 7.41 mmole) was dissolved in DMF (14 mL). 4-(Aminomethyl)benzonitrile hydrochloride (1.027 g, 7.77 mmole) and N,N-diisopropylethylamine (1.90 mL, 10.9 mmole) were added and the solution was heated to 70° C. for 2½ days, then was cooled to RT. Ethyl acetate (40 mL) was added and the mixture was washed with water (3×25 mL). The organics were dried over magnesium sulfate, filtered, and concentrated. The crude residue was purified by silica gel chromatography (40-80% ethyl acetate/hexane gradient) to provide 2-methoxyethyl (4-cyanobenzyl)carbamate as a yellow solid (0.888 g, 51%).

¹H NMR (400 MHz, CDCl₃): δ 7.67-7.58 (m, 2H), 7.40 (dq, J=7.4, 0.8 Hz, 2H), 5.23 (s, 1H), 4.43 (d, J=6.3 Hz, 2H), 4.31-4.24 (m, 2H), 3.64-3.53 (m, 2H), 3.40 (s, 3H).

### 2-Methoxyethyl (4-carbamothioylbenzyl)carbamate (26.3)

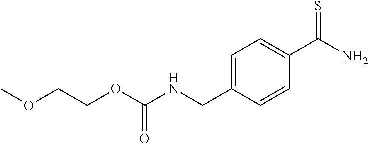 [SMILES: COCCOC(=O)NCc1ccc(C(N)=S)cc1]

2-Methoxyethyl (4-cyanobenzyl)carbamate (0.888 g, 3.79 mmole) was dissolved in pyridine (4 mL). Triethylamine (0.590 mL, 4.21 mmole) and ammonium sulfide (40% aqueous solution, 0.780 mL, 4.57 mmole) were added and the mixture was warmed to 50° C. for 5 hours, then was cooled to RT and concentrated. The residue was suspended in ethyl acetate (25 mL). The mixture was washed sequentially with 1N aq. HCl (2×15 mL) and water (1×15 mL). The aqueous layers were extracted with ethyl acetate (1×10 mL). The combined organics were dried over magnesium sulfate, filtered, and concentrated to provide 2-methoxyethyl (4-carbamothioylbenzyl)carbamate as a yellow solid (0.932 g, 92%).

¹H NMR (400 MHz, DMSO-d₆): δ 9.81 (s, 1H), 9.44 (s, 1H), 7.87-7.82 (m, 2H), 7.82-7.75 (m, 1H), 7.27 (d, J=8.3 Hz, 2H), 4.20 (d, J=6.2 Hz, 2H), 4.12-4.04 (m, 2H), 3.53-3.44 (m, 2H), 3.25 (s, 3H).

### Ethyl 2-(4-((((2-methoxyethoxy)carbonyl)amino)methyl)phenyl)thiazole-4-carboxylate (26.4)

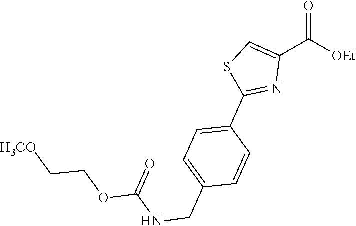 [SMILES: CCOC(=O)c1csc(-c2ccc(CNC(=O)OCCOC)cc2)n1]

2-Methoxyethyl (4-carbamothioylbenzyl)carbamate (0.932 g, 3.47 mmole) was dissolved in ethanol (7 mL). Ethyl bromopyruvate (0.480 mL, 3.83 mmole) was added and the mixture was heated to 80° C. for 2½ hours. The mixture was cooled to RT and concentrated. The residue was suspended in ethyl acetate (25 mL) and washed with water (1×25 mL), sat. aq. sodium bicarbonate (1×25 mL), water (1×25 mL), and sat. aq. sodium chloride (1×25 mL). The organics were dried over magnesium sulfate, filtered, and concentrated. The crude residue was purified by silica gel chromatography (40-80% ethyl acetate/hexane gradient) to provide ethyl 2-(4-((((2-methoxyethoxy)carbonyl)amino)methyl)phenyl)thiazole-4-carboxylate as a white solid (0.887 g, 70%).

¹H NMR (400 MHz, CDCl₃): δ 8.15 (s, 1H), 8.03-7.92 (m, 2H), 7.39-7.33 (m, 2H), 5.16 (s, 1H), 4.49-4.39 (m, 4H), 4.32-4.23 (m, 2H), 3.64-3.56 (m, 2H), 3.40 (s, 3H), 1.43 (t, J=7.1 Hz, 3H).

### Methyl O-(tert-butyldimethylsilyl)-N-(2-(4-((((2-methoxyethoxy)carbonyl)amino)methyl)phenyl)thiazole-4-carbonyl)-L-serinate (26.5)

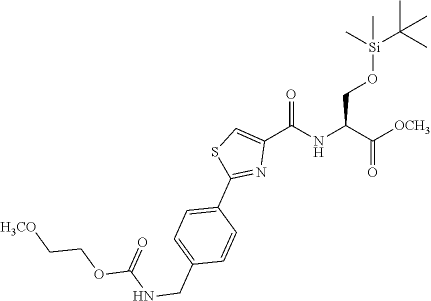 [SMILES: COCCOC(=O)NCc1ccc(-c2nc(C(=O)N[C@@H](CO[Si](C)(C)C(C)(C)C)C(=O)OC)cs2)cc1]

Using the procedure described in Example 19.3, ethyl 2-(4-((((2-methoxyethoxy)carbonyl)amino)methyl)phenyl)thiazole-4-carboxylate (0.887 g, 2.43 mmole) was converted to methyl O-(tert-butyldimethylsilyl)-N-(2-(4-((((2-methoxyethoxy)carbonyl)amino)methyl)phenyl)thiazole-4-carbonyl)-L-serinate as a thick, colorless oil (1.202 g, 90%).

¹H NMR (400 MHz, CDCl₃): δ 8.22 (d, J=8.7 Hz, 1H), 8.09 (s, 1H), 7.97-7.88 (m, 2H), 7.40-7.34 (m, 2H), 5.21 (s, 1H), 4.90-4.82 (m, 1H), 4.43 (dd, J=6.3, 2.1 Hz, 2H), 4.33-4.25 (m, 2H), 4.21 (dd, J=10.1, 2.6 Hz, 1H), 3.95 (dd, J=10.1, 3.4 Hz, 1H), 3.79 (s, 3H), 3.65-3.56 (m, 2H), 3.40 (s, 3H), 0.92 (s, 9H), 0.08 (s, 3H), 0.06 (s, 3H).

### Methyl O-acetyl-N—(O-(tert-butyldimethylsilyl)-N-(2-(4-((((2-methoxyethoxy)carbonyl)amino)methyl)phenyl)thiazole-4-carbonyl)-L-seryl)-L-serinate (26.6)

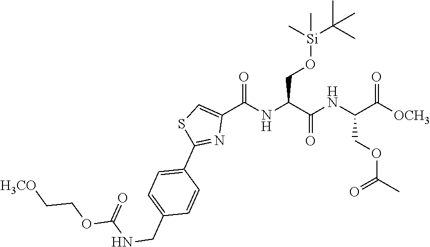 [SMILES: COCCOC(=O)NCc1ccc(-c2nc(C(=O)N[C@@H](CO[Si](C)(C)C(C)(C)C)C(=O)N[C@@H](COC(C)=O)C(=O)OC)cs2)cc1]

Using the procedure described in Example 15.6, methyl O-(tert-butyldimethylsilyl)-N-(2-(4-((((2-methoxyethoxy)carbonyl)amino)methyl)phenyl)thiazole-4-carbonyl)-L-serinate (1.202 g, 2.18 mmole) was converted to methyl O-acetyl-N—(O-(tert-butyldimethylsilyl)-N-(2-(4-((((2-methoxyethoxy)carbonyl)amino)methyl)phenyl)thiazole-4-carbonyl)-L-seryl)-L-serinate as a colorless oil (1.001 g, 67%).

¹H NMR (400 MHz, CDCl₃): δ 8.26 (d, J=7.1 Hz, 1H), 8.11 (d, J=2.6 Hz, 1H), 7.94 (dd, J=8.3, 1.8 Hz, 2H), 7.46 (d, J=7.8 Hz, 1H), 7.41-7.32 (m, 2H), 5.17 (s, 1H), 4.90 (dq, J=7.0, 3.5 Hz, 1H), 4.71-4.62 (m, 1H), 4.49 (ddd, J=11.2, 7.2, 3.9 Hz, 1H), 4.43 (d, J=6.2 Hz, 2H), 4.33 (dd, J=11.4, 3.6 Hz, 1H), 4.31-4.26 (m, 2H), 4.23 (dt, J=9.9, 3.6 Hz, 1H), 3.84-3.79 (m, 1H), 3.77 (d, J=4.0 Hz, 3H), 3.66-3.57 (m, 2H), 3.41 (s, 3H), 2.01 (s, 3H), 0.96 (s, 9H), 0.16 (s, 3H), 0.16 (s, 3H).

### Methyl 2-(2-(2-(4-((((2-methoxyethoxy)carbonyl)amino)methyl)phenyl)thiazole-4-carboxamido)acrylamido)acrylate (26)

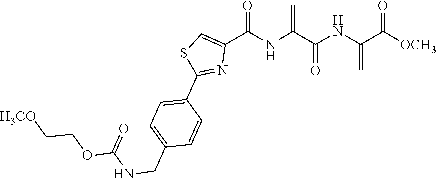 [SMILES: C=C(NC(=O)c1csc(-c2ccc(CNC(=O)OCCOC)cc2)n1)C(=O)NC(=C)C(=O)OC]

Using the procedure described for Compound 15, methyl O-acetyl-N—(O-(tert-butyldimethylsilyl)-N-(2-(4-((((2-methoxyethoxy)carbonyl)amino)methyl)phenyl)thiazole-4-carbonyl)-L-seryl)-L-serinate (1.001 g, 1.47 mmole) was converted to methyl 2-(2-(2-(4-((((2-methoxyethoxy)carbonyl)amino)methyl)phenyl)thiazole-4-carboxamido)acrylamido)acrylate as a white solid (0.103 g, 14%).

¹H NMR (400 MHz, CDCl₃): δ 10.01 (s, 1H), 8.55 (s, 1H), 8.14 (s, 1H), 8.04-7.92 (m, 2H), 7.44-7.33 (m, 2H), 6.78 (d, J=2.2 Hz, 1H), 6.71 (s, 1H), 6.03 (d, J=1.3 Hz, 1H), 5.50 (t, J=1.9 Hz, 1H), 5.18 (s, 1H), 4.43 (d, J=6.1 Hz, 2H), 4.34-4.24 (m, 2H), 3.90 (s, 3H), 3.67-3.56 (m, 2H), 3.41 (s, 3H).

### Compound 27: Methyl 2-(2-(2-(3-((2-methoxyethoxy)methyl)phenyl)thiazole-4-carboxamido)acrylamido)acrylate

### 3-((2-Methoxyethoxy)methyl)benzonitrile (27.1)

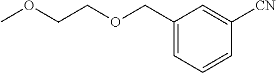

2-Methoxyethanol (1.05 mL, 13.3 mmole) was dissolved in DMF (20 mL) and cooled to 0° C. Sodium hydride (60% dispersion in mineral oil, 0.532 g, 13.3 mmole) was added portionwise (gas evolution was observed) and the mixture was stirred at 0° for 60 minutes. 3-(Bromomethyl)benzonitrile (2.046 g, 10.4 mmole) was added and the mixture was warmed to RT and stirred for 18 hours. Ethyl acetate (50 mL) was added and the mixture was washed with water (3×25 mL). The combined organics were dried over magnesium sulfate, filtered, and concentrated. The crude residue was purified by silica gel chromatography (10-40% ethyl acetate/hexane gradient) to provide 3-((2-methoxyethoxy)methyl)benzonitrile as a colorless oil (0.844, 42%).

¹H NMR (400 MHz, CDCl₃): δ 7.67 (td, J=1.7, 0.8 Hz, 1H), 7.62-7.54 (m, 2H), 7.45 (t, J=7.7 Hz, 1H), 4.64-4.56 (m, 2H), 3.71-3.63 (m, 2H), 3.63-3.56 (m, 2H), 3.41 (s, 3H).

### 3-((2-Methoxyethoxy)methyl)benzothioamide (27.2)

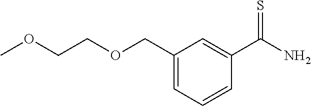

3-((2-Methoxyethoxy)methyl)benzonitrile (0.844 g, 4.41 mmole) was dissolved in pyridine (5 mL). Triethylamine (0.740 mL, 5.28 mmole) and ammonium sulfide (40% aqueous solution, 1.05 mL, 6.15 mmole) were added and the mixture was heated to 50° C. for 5 hours and was cooled to RT. The mixture was concentrated and the residue was suspended in ethyl acetate (25 mL). The mixture was washed with 1N aq. HCl (2×25 mL), then dried over magnesium sulfate, filtered, and concentrated to provide 3-((2-methoxyethoxy)methyl)benzothioamide as a yellow oil (0.797 g, 80%).

¹H NMR (400 MHz, DMSO-d₆): δ 9.86 (s, 1H), 9.50 (s, 1H), 7.85 (q, J=1.4, 0.9 Hz, 1H), 7.80-7.72 (m, 1H), 7.45 (dt, J=7.6, 1.5 Hz, 1H), 7.39 (t, J=7.6 Hz, 1H), 4.52 (s, 2H), 3.62-3.54 (m, 2H), 3.53-3.45 (m, 2H), 3.26 (s, 3H).

### Ethyl 2-(3-((2-methoxyethoxy)methyl)phenyl)thiazole-4-carboxylate (27.3)

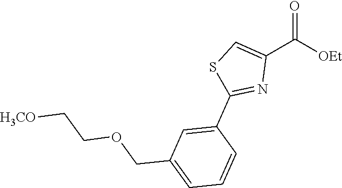 [SMILES: CCOC(=O)c1csc(-c2cccc(COCCOC)c2)n1]

3-((2-Methoxyethoxy)methyl)benzothioamide (0.797 g, 3.54 mmole) was dissolved in ethanol. Ethyl bromopyruvate (0.490 mL, 3.90 mmole) was added and the mixture was heated to 80° C. for 4 hours. The mixture was cooled to RT and concentrated. The crude residue was purified by silica gel chromatography (20-50% ethyl acetate/hexane gradient) to provide ethyl 2-(3-((2-methoxyethoxy)methyl)phenyl)thiazole-4-carboxylate as a yellow oil (0.421 g, 37%).

¹H NMR (400 MHz, CDCl₃): δ 8.16 (s, 1H), 8.00 (td, J=1.7, 0.7 Hz, 1H), 7.92 (dt, J=7.4, 1.7 Hz, 1H), 7.51-7.39 (m, 2H), 4.64 (s, 2H), 4.45 (q, J=7.1 Hz, 2H), 3.70-3.62 (m, 2H), 3.62-3.55 (m, 2H), 3.41 (s, 3H), 1.44 (t, J=7.1 Hz, 3H).

### Methyl O-(tert-butyldimethylsilyl)-N-(2-(3-((2-methoxyethoxy)methyl)phenyl)thiazole-4-carbonyl)-L-serinate (27.4)

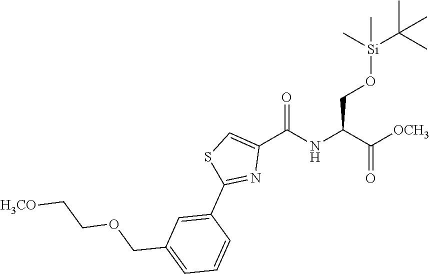 [SMILES: COCCOCc1cccc(-c2nc(C(=O)N[C@@H](CO[Si](C)(C)C(C)(C)C)C(=O)OC)cs2)c1]

Using the procedure described in Example 19.3, ethyl 2-(3-((2-methoxyethoxy)methyl)phenyl)thiazole-4-carboxylate (0.421 g, 1.31 mmole) was converted to methyl O-(tert-butyldimethylsilyl)-N-(2-(3-((2-methoxyethoxy)methyl)phenyl)thiazole-4-carbonyl)-L-serinate as a thick, colorless oil (0.475 g, 71%).

¹H NMR (400 MHz, CDCl₃): δ 8.21 (d, J=8.7 Hz, 1H), 8.11 (s, 1H), 7.95-7.87 (m, 2H), 7.52-7.40 (m, 2H), 4.87 (ddd, J=8.7, 3.4, 2.7 Hz, 1H), 4.65 (s, 2H), 4.22 (dd, J=10.1, 2.7 Hz, 1H), 3.95 (dd, J=10.1, 3.4 Hz, 1H), 3.79 (s, 3H), 3.71-3.64 (m, 2H), 3.64-3.58 (m, 2H), 3.41 (s, 3H), 0.92 (s, 9H), 0.08 (s, 3H), 0.06 (s, 3H).

### Methyl O-acetyl-N—(O-(tert-butyldimethylsilyl)-N-(2-(3-((2-methoxyethoxy)methyl)phenyl)thiazole-4-carbonyl)-L-seryl)-L-serinate (27.5)

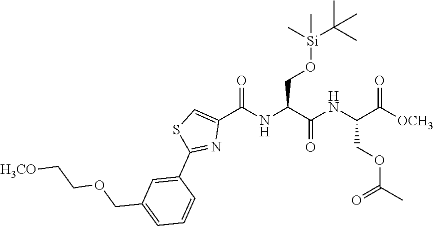 [SMILES: COCCOCc1cccc(-c2nc(C(=O)N[C@@H](CO[Si](C)(C)C(C)(C)C)C(=O)N[C@@H](COC(C)=O)C(=O)OC)cs2)c1]

Using the procedure described in Example 15.6, methyl O-(tert-butyldimethylsilyl)-N-(2-(3-((2-methoxyethoxy)methyl)phenyl)thiazole-4-carbonyl)-L-serinate (0.475 g, 0.934 mmole) was converted to methyl O-acetyl-N—(O-(tert-butyldimethylsilyl)-N-(2-(3-((2-methoxyethoxy)methyl)phenyl)thiazole-4-carbonyl)-L-seryl)-L-serinate as a colorless oil (0.415 g, 70%).

¹H NMR (400 MHz, CDCl₃): δ 8.24 (d, J=7.1 Hz, 1H), 8.12 (d, J=2.5 Hz, 1H), 7.92 (ddt, J=5.2, 3.4, 1.7 Hz, 2H), 7.51-7.40 (m, 3H), 4.95-4.85 (m, 1H), 4.72-4.66 (m, 1H), 4.65 (s, 2H), 4.49 (ddd, J=11.3, 6.1, 3.9 Hz, 1H), 4.34 (dd, J=11.4, 3.6 Hz, 1H), 4.22 (ddd, J=9.8, 3.8, 2.8 Hz, 1H), 3.84-3.79 (m, 1H), 3.77 (s, 3H), 3.70-3.64 (m, 2H), 3.64-3.56 (m, 2H), 3.41 (s, 3H), 2.02 (s, 3H), 0.96 (s, 9H), 0.17 (s, 3H), 0.16 (s, 3H).

### Methyl 2-(2-(2-(3-((2-methoxyethoxy)methyl)phenyl)thiazole-4-carboxamido)acrylamido)acrylate (27)

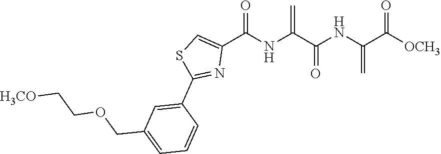 [SMILES: C=C(NC(=O)c1csc(-c2cccc(COCCOC)c2)n1)C(=O)NC(=C)C(=O)OC]

Using the procedure as described for Compound 15, methyl O-acetyl-N—(O-(tert-butyldimethylsilyl)-N-(2-(3-((2-methoxyethoxy)methyl)phenyl)thiazole-4-carbonyl)-L-seryl)-L-serinate (0.415 g, 0.651 mmole) was converted to methyl 2-(2-(2-(3-((2-methoxyethoxy)methyl)phenyl)thiazole-4-carboxamido)acrylamido)acrylate as a white solid (0.153 g, 53%).

¹H NMR (400 MHz, CDCl₃): δ 10.00 (s, 1H), 8.55 (s, 1H), 8.16 (s, 1H), 7.97 (dt, J=6.5, 2.3 Hz, 1H), 7.94 (d, J=1.7 Hz, 1H), 7.51-7.42 (m, 2H), 6.78 (d, J=2.2 Hz, 1H), 6.72 (s, 1H), 6.03 (d, J=1.3 Hz, 1H), 5.51 (t, J=1.9 Hz, 1H), 4.67 (s, 2H), 3.90 (s, 3H), 3.74-3.66 (m, 2H), 3.66-3.57 (m, 2H), 3.42 (s, 3H).

### Compound 28: Methyl 2-(2-(2-(3-(isobutylcarbamoyl)piperidin-1-yl)thiazole-4-carboxamido)acrylamido)acrylate

### Tert-butyl 3-(isobutylcarbamoyl)piperidine-1-carboxylate (28.1)

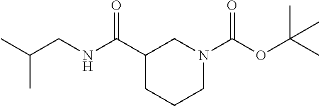 [SMILES: CC(C)CNC(=O)C1CCCN(C(=O)OC(C)(C)C)C1]

1-(Tert-butoxycarbonyl)piperidine-3-carboxylic acid (0.754 g, 3.29 mmole) was dissolved in DCM (6.5 mL). Isobutylamine (0.390 mL, 3.92 mmole) and N,N-diisopropylethylamine (1.15 mL, 6.60 mmole) were added followed by HOBt·H2O (0.603 g, 3.94 mmole) and EDC·HCl (0.750 g, 3.91 mmole). The mixture was stirred at RT for 3 days. Water (25 mL) was added and the mixture was extracted with DCM (3×10 mL). The combined organics were dried over magnesium sulfate, filtered, and concentrated. The crude residue was purified by silica gel chromatography (20-50% ethyl acetate/hexane gradient) to provide tert-butyl 3-(isobutylcarbamoyl)piperidine-1-carboxylate as a white solid (0.817 g, 87%).

¹H NMR (400 MHz, CDCl₃): δ 4.07-3.61 (m, 2H), 3.26 (s, 1H), 3.08 (t, J=6.4 Hz, 2H), 2.29 (s, 1H), 1.85 (d, J=29.1 Hz, 2H), 1.76 (p, J=6.7 Hz, 1H), 1.62 (s, 1H), 1.46 (s, 9H), 0.91 (dd, J=6.7, 1.1 Hz, 6H).

### Ethyl 2-(3-(isobutylcarbamoyl)piperidin-1-yl)thiazole-4-carboxylate (28.2)

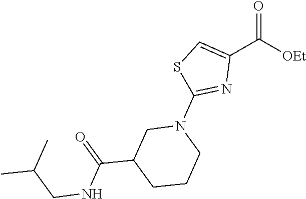 [SMILES: CCOC(=O)c1csc(N2CCCC(C(=O)NCC(C)C)C2)n1]

Tert-butyl 3-(isobutylcarbamoyl)piperidine-1-carboxylate (0.817 g, 2.87 mmole) was dissolved in DCM (5.5 mL). HCl (4M solution in 1,4-dioxane, 2.80 mL, 11.2 mmole) was added and the mixture was stirred at RT for 3 hours, then was concentrated. The residue was dissolved in DMA (3 mL) and ethyl 2-bromothiazole-4-carboxylate (0.749 g, 3.17 mmole) and triethylamine (0.490 mL, 3.50 mmole) were added. The mixture was heated to 80° C. for 18 hours and was cooled to RT. Ethyl acetate (25 mL) was added and the mixture was washed with water (3×25 mL). The organics were dried over magnesium sulfate, filtered, and concentrated. The crude residue was purified by silica gel chromatography (30-70% ethyl acetate/hexane gradient) to provide ethyl 2-(3-(isobutylcarbamoyl)piperidin-1-yl)thiazole-4-carboxylate as a white solid (0.445 g, 46%).

¹H NMR (400 MHz, CDCl₃): δ 7.43 (s, 1H), 5.93 (s, 1H), 4.35 (q, J=7.1 Hz, 2H), 4.05 (ddd, J=13.3, 3.2, 2.0 Hz, 1H), 3.83-3.74 (m, 1H), 3.47 (dd, J=13.2, 9.5 Hz, 1H), 3.24-3.14 (m, 1H), 3.14-3.01 (m, 2H), 2.45 (tt, J=9.3, 4.5 Hz, 1H), 1.93 (ddd, J=9.5, 8.0, 4.3 Hz, 2H), 1.85-1.71 (m, 2H), 1.68-1.59 (m, 1H), 1.37 (t, J=7.1 Hz, 3H), 0.89 (d, J=6.7 Hz, 6H).

### Methyl O-(tert-butyldimethylsilyl)-N-(2-(3-(isobutylcarbamoyl)piperidin-1-yl)thiazole-4-carbonyl)-L-serinate (28.3)

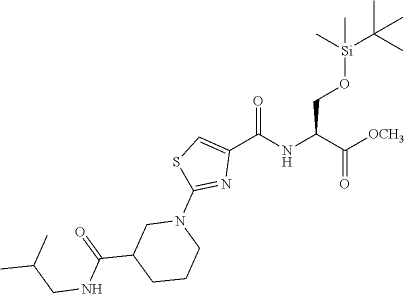 [SMILES: COC(=O)[C@H](CO[Si](C)(C)C(C)(C)C)NC(=O)c1csc(N2CCCC(C(=O)NCC(C)C)C2)n1]

Using the procedure described in Example 19.3, ethyl 2-(3-(isobutylcarbamoyl)piperidin-1-yl)thiazole-4-carboxylate (0.445 g, 1.34 mmole) was converted to a diastereomeric mixture of methyl O-(tert-butyldimethylsilyl)-N-(2-(3-(isobutylcarbamoyl)piperidin-1-yl)thiazole-4-carbonyl)-L-serinate as a thick, colorless oil (0.470 g, 67%).

¹H NMR (400 MHz, CDCl₃): δ 7.93 (dd, J=17.6, 8.7 Hz, 1H), 7.38 (d, J=2.1 Hz, 1H), 5.84 (dd, J=13.7, 6.7 Hz, 1H), 4.79 (ddt, J=9.5, 6.4, 3.0 Hz, 1H), 4.19-4.13 (m, 1H), 3.99 (dd, J=13.2, 4.0 Hz, 1H), 3.93-3.79 (m, 2H), 3.76 (d, J=0.8 Hz, 3H), 3.47 (ddd, J=48.4, 13.3, 9.5 Hz, 1H), 3.25-3.00 (m, 3H), 2.46 (qt, J=8.7, 4.1 Hz, 1H), 1.93 (dq, J=11.6, 3.6, 2.3 Hz, 2H), 1.77 (dtd, J=13.4, 6.8, 5.1 Hz, 2H), 1.67 (dt, J=9.2, 4.6 Hz, 2H), 0.90 (dd, J=6.7, 4.7 Hz, 6H), 0.87 (d, J=1.6 Hz, 9H), 0.04 (d, 3H), 0.02 (d, 3H).

### Methyl O-acetyl-N—(O-(tert-butyldimethylsilyl)-N-(2-(3-(isobutylcarbamoyl)piperidin-1-yl)thiazole-4-carbonyl)-L-seryl)-L-serinate (28.4)

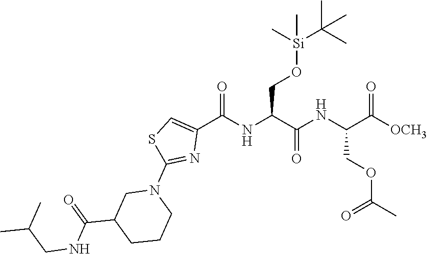 [SMILES: COC(=O)[C@H](COC(C)=O)NC(=O)[C@H](CO[Si](C)(C)C(C)(C)C)NC(=O)c1csc(N2CCCC(C(=O)NCC(C)C)C2)n1]

Using the procedure described in Example 15.6, methyl O-(tert-butyldimethylsilyl)-N-(2-(3-(isobutylcarbamoyl)piperidin-1-yl)thiazole-4-carbonyl)-L-serinate (0.470 g, 0.892 mmole) was converted to methyl O-acetyl-N—(O-(tert-butyldimethylsilyl)-N-(2-(3-(isobutylcarbamoyl)piperidin-1-yl)thiazole-4-carbonyl)-L-seryl)-L-serinate as a colorless oil (0.422 g, 72%).

¹H NMR (400 MHz, CDCl₃): δ 7.98 (dd, J=16.3, 6.9 Hz, 1H), 7.46 (dd, J=14.0, 7.9 Hz, 1H), 7.40 (d, J=1.3 Hz, 1H), 6.03-5.72 (m, 1H), 4.86 (ddt, J=7.6, 5.4, 3.8 Hz, 1H), 4.62-4.53 (m, 1H), 4.47 (ddd, J=11.3, 8.4, 3.9 Hz, 1H), 4.37-4.25 (m, 1H), 4.22-4.15 (m, 1H), 3.88 (dd, J=13.1, 4.5 Hz, 1H), 3.81-3.68 (m, 4H), 3.50-3.32 (m, 1H), 3.25-2.99 (m, 3H), 2.47 (ddt, J=15.5, 5.8, 3.4 Hz, 1H), 2.04-1.99 (m, 3H), 1.98-1.87 (m, 2H), 1.79 (ttt, J=13.5, 6.7, 3.1 Hz, 2H), 0.92 (d, J=1.6 Hz, 8H), 0.90 (dd, J=6.6, 0.8 Hz, 6H), 0.14 (dt, J=6.1, 3.3 Hz, 6H).

### Methyl 2-(2-(2-(3-(isobutylcarbamoyl)piperidin-1-yl)thiazole-4-carboxamido)acrylamido)acrylate (28)

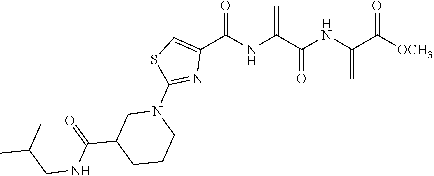 [SMILES: C=C(NC(=O)c1csc(N2CCCC(C(=O)NCC(C)C)C2)n1)C(=O)NC(=C)C(=O)OC]

Using the procedure as described for Compound 15, methyl O-acetyl-N—(O-(tert-butyldimethylsilyl)-N-(2-(3-(isobutylcarbamoyl)piperidin-1-yl)thiazole-4-carbonyl)-L-seryl)-L-serinate (0.422 g, 0.643 mmole) was converted to methyl 2-(2-(2-(3-(isobutylcarbamoyl)piperidin-1-yl)thiazole-4-carboxamido)acrylamido)acrylate as a white solid (0.139 g, 47%).

¹H NMR (400 MHz, CDCl₃): δ 9.78 (s, 1H), 8.52 (s, 1H), 7.44 (s, 1H), 6.71 (d, J=2.1 Hz, 1H), 6.63 (s, 1H), 6.00 (d, J=1.3 Hz, 1H), 5.92 (t, J=6.0 Hz, 1H), 5.43 (t, J=1.9 Hz, 1H), 4.07 (dd, J=13.3, 4.0 Hz, 1H), 3.90 (s, 3H), 3.77 (ddd, J=13.7, 5.1, 3.8 Hz, 1H), 3.47-3.37 (m, 1H), 3.23 (ddd, J=13.2, 11.0, 3.4 Hz, 1H), 3.19-3.04 (m, 2H), 2.60-2.47 (m, 1H), 1.96 (td, J=8.3, 7.7, 3.9 Hz, 2H), 1.87-1.73 (m, 2H), 1.73-1.62 (m, 1H), 0.90 (d, J=6.7 Hz, 6H).

### Compound 29: Methyl 2-(2-(2-(4-((3-isopropoxypropyl)carbamoyl)piperidin-1-yl)thiazole-4-carboxamido)acrylamido)acrylate

### Ethyl 2-(4-(tert-butoxycarbonyl)piperidin-1-yl)thiazole-4-carboxylate (29.1)

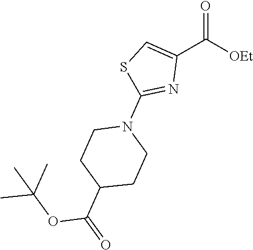 [SMILES: CCOC(=O)c1csc(N2CCC(C(=O)OC(C)(C)C)CC2)n1]

Tert-butyl piperidine-4-carboxylate hydrochloride (2.000 g, 9.02 mmole) was dissolved in DMA (10 mL). Ethyl 2-bromothiazole-4-carboxylate (2.251 g, 9.53 mmole) and triethylamine (1.40 mL, 9.99 mmole) were added and the mixture was heated to 80° C. for 18 hours, then was cooled to RT. Ethyl acetate (50 mL) was added and the mixture was washed with water (3×25 mL). The organics were dried over magnesium sulfate, filtered, and concentrated. The crude residue was purified by silica gel chromatography (10-30% ethyl acetate/hexane gradient) to provide ethyl 2-(4-(tert-butoxycarbonyl)piperidin-1-yl)thiazole-4-carboxylate as a pale yellow solid (1.735 g, 57%).

¹H NMR (400 MHz, CDCl₃): δ 7.43 (s, 1H), 4.35 (q, J=7.1 Hz, 2H), 4.02-3.91 (m, 2H), 3.14 (ddd, J=13.0, 11.1, 3.2 Hz, 2H), 2.43 (tt, J=10.7, 3.8 Hz, 1H), 1.98 (dtt, J=13.5, 3.9, 2.0 Hz, 2H), 1.85-1.73 (m, 2H), 1.45 (s, 9H), 1.37 (t, J=7.1 Hz, 3H).

### Ethyl 2-(4-((3-isopropoxypropyl)carbamoyl)piperidin-1-yl)thiazole-4-carboxylate (29.2)

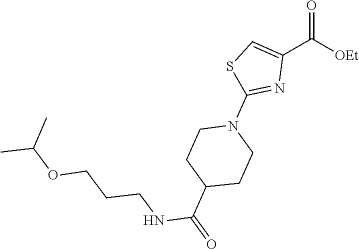 [SMILES: CCOC(=O)c1csc(N2CCC(C(=O)NCCCOC(C)C)CC2)n1]

Ethyl 2-(4-(tert-butoxycarbonyl)piperidin-1-yl)thiazole-4-carboxylate (0.557 g, 1.64 mmole) was dissolved in DCM (3.2 mL). HCl (4M solution in 1,4-dioxane, 1.60 mL, 6.40 mmole) was added and the solution was stirred at RT for 18 hours, then was concentrated. The residue was suspended in DCM (3.2 mL). N,N-Diisopropylethylamine (0.560 mL, 3.22 mmole) was added followed by 3-isopropoxypropan-1-amine (0.270 mL, 1.95 mmole), HOBt·H₂O (0.295 g, 1.93 mmole), and EDC·HCl (0.382 g, 1.99 mmole). The mixture was stirred at RT for 18 hours and water (25 mL) was added. The mixture was extracted with DCM (3×10 mL). The combined organics were dried over magnesium sulfate, filtered, and concentrated. The crude residue was purified by silica gel chromatography (70-100% ethyl acetate/hexane gradient) to provide ethyl 2-(4-((3-isopropoxypropyl)carbamoyl)piperidin-1-yl)thiazole-4-carboxylate as a white solid (0.497 g, 79%).

¹H NMR (400 MHz, CDCl₃): δ 7.44 (s, 1H), 6.46 (s, 1H), 4.35 (q, J=7.1 Hz, 2H), 4.08 (dt, J=13.4, 4.2 Hz, 2H), 3.62-3.50 (m, 3H), 3.44-3.34 (m, 2H), 3.07 (ddd, J=13.0, 11.8, 3.1 Hz, 2H), 2.27 (tt, J=11.5, 3.6 Hz, 1H), 2.02-1.92 (m, 2H), 1.86-1.71 (m, 4H), 1.37 (t, J=7.1 Hz, 3H), 1.16 (d, J=6.1 Hz, 6H).

### Methyl O-(tert-butyldimethylsilyl)-N-(2-(4-((3-isopropoxypropyl)carbamoyl)piperidin-1-yl)thiazole-4-carbonyl)-L-serinate (29.3)

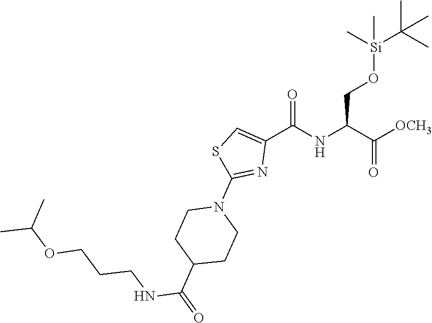 [SMILES: COC(=O)[C@H](CO[Si](C)(C)C(C)(C)C)NC(=O)c1csc(N2CCC(C(=O)NCCCOC(C)C)CC2)n1]

Using the procedure described in Example 19.3, ethyl 2-(4-((3-isopropoxypropyl)carbamoyl)piperidin-1-yl)thiazole-4-carboxylate (0.497 g, 1.30 mmole) was converted to methyl O-(tert-butyldimethylsilyl)-N-(2-(4-((3-isopropoxypropyl)carbamoyl)piperidin-1-yl)thiazole-4-carbonyl)-L-serinate as a thick, colorless oil (0.418 g, 56%).

¹H NMR (400 MHz, CDCl₃): δ 7.96 (d, J=8.8 Hz, 1H), 7.38 (s, 1H), 6.48 (t, J=5.2 Hz, 1H), 4.78 (ddd, J=8.8, 3.4, 2.6 Hz, 1H), 4.19-4.14 (m, 1H), 4.08-3.96 (m, 2H), 3.88 (dd, J=10.0, 3.4 Hz, 1H), 3.76 (s, 3H), 3.62-3.51 (m, 3H), 3.44-3.35 (m, 2H), 3.12-2.98 (m, 2H), 2.28 (tt, J=11.5, 3.6 Hz, 1H), 2.01-1.91 (m, 2H), 1.87-1.70 (m, 5H), 1.17 (d, J=6.1 Hz, 6H), 0.88 (s, 9H), 0.05 (s, 3H), 0.03 (s, 3H).

### Methyl O-acetyl-N—(O-(tert-butyldimethylsilyl)-N-(2-(4-((3-isopropoxypropyl)carbamoyl)piperidin-1-yl)thiazole-4-carbonyl)-L-seryl)-L-serinate (29.4)

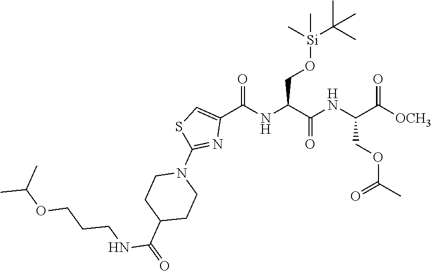 [SMILES: COC(=O)[C@H](COC(C)=O)NC(=O)[C@H](CO[Si](C)(C)C(C)(C)C)NC(=O)c1csc(N2CCC(C(=O)NCCCOC(C)C)CC2)n1]

Using the procedure described in Example 15.6, methyl O-(tert-butyldimethylsilyl)-N-(2-(4-((3-isopropoxypropyl)carbamoyl)piperidin-1-yl)thiazole-4-carbonyl)-L-serinate (0.418 g, 0.732 mmole) was converted to methyl O-acetyl-N—(O-(tert-butyldimethylsilyl)-N-(2-(4-((3-isopropoxypropyl)carbamoyl)piperidin-1-yl)thiazole-4-carbonyl)-L-seryl)-L-serinate as a colorless oil (0.276 g, 54%).

¹H NMR (400 MHz, CDCl₃): δ 7.99 (d, J=7.1 Hz, 1H), 7.43 (d, J=7.8 Hz, 1H), 7.39 (d, J=3.0 Hz, 1H), 6.47 (t, J=5.1 Hz, 1H), 4.87 (dq, J=8.2, 4.0 Hz, 1H), 4.63-4.54 (m, 1H), 4.51-4.43 (m, 1H), 4.31 (dd, J=11.4, 3.7 Hz, 1H), 4.23-4.16 (m, 1H), 4.03 (tt, J=15.8, 2.7 Hz, 2H), 3.76 (d, J=2.6 Hz, 3H), 3.75-3.69 (m, 1H), 3.62-3.52 (m, 3H), 3.40 (dt, J=6.0, 5.0 Hz, 2H), 3.05 (td, J=12.6, 3.0 Hz, 2H), 2.28 (tt, J=11.5, 3.6 Hz, 1H), 2.02 (d, J=2.8 Hz, 3H), 2.00-1.90 (m, 2H), 1.85-1.72 (m, 4H), 1.17 (d, J=6.1 Hz, 6H), 0.92 (s, 9H), 0.14 (s, 3H), 0.13 (s, 3H).

### Methyl 2-(2-(2-(4-((3-isopropoxypropyl)carbamoyl)piperidin-1-yl)thiazole-4-carboxamido)acrylamido)acrylate (29)

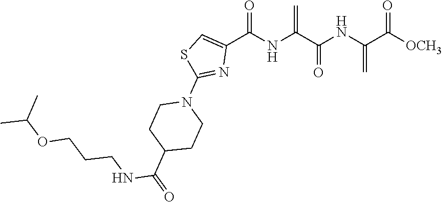 [SMILES: C=C(NC(=O)c1csc(N2CCC(C(=O)NCCCOC(C)C)CC2)n1)C(=O)NC(=C)C(=O)OC]

Using the procedure as described for Compound 15, methyl O-acetyl-N—(O-(tert-butyldimethylsilyl)-N-(2-(4-((3-isopropoxypropyl)carbamoyl)piperidin-1-yl)thiazole-4-carbonyl)-L-seryl)-L-serinate (0.276 g, 0.394 mmole) was converted to methyl 2-(2-(2-(4-((3-isopropoxypropyl)carbamoyl)piperidin-1-yl)thiazole-4-carboxamido)acrylamido)acrylate as a white solid (0.088 g, 44%).

¹H NMR (400 MHz, CDCl₃): δ 9.72 (s, 1H), 8.51 (s, 1H), 7.43 (s, 1H), 6.72 (d, J=2.1 Hz, 1H), 6.66 (s, 1H), 6.46 (s, 1H), 6.00 (d, J=1.3 Hz, 1H), 5.43 (t, J=1.8 Hz, 1H), 4.10-4.01 (m, 2H), 3.89 (s, 3H), 3.63-3.50 (m, 3H), 3.46-3.34 (m, 2H), 3.08 (ddd, J=13.0, 11.8, 3.0 Hz, 2H), 2.29 (tt, J=11.5, 3.7 Hz, 1H), 2.02-1.92 (m, 2H), 1.89-1.72 (m, 4H), 1.17 (d, J=6.1 Hz, 6H).

### Compound 30: Methyl 2-(2-(2-(4-(((benzylcarbamoyl)oxy)methyl)phenyl)thiazole-4-carboxamido)acrylamido)acrylate

### 4-Cyanobenzyl 1H-imidazole-1-carboxylate (30.1)

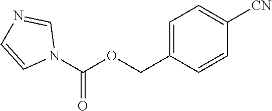 [SMILES: N#Cc1ccc(COC(=O)n2ccnc2)cc1]

Following a literature procedure (RSC Adv., 2014, 4, 13012-13017), 4-(hydroxymethyl)benzonitrile (2.000 g, 15.0 mmole) was dissolved in THF (30 mL). N,N′-Carbonyldiimidazole (2.924 g, 18.0 mmole) was added and the solution was stirred at RT for 2½ days. Water (50 mL) was added and the mixture was extracted with ethyl acetate (3×15 mL). The combined organics were washed sequentially with water (1×25 mL) and sat. aq. sodium chloride (1×25 mL), then dried over magnesium sulfate, filtered, and concentrated to provide 4-cyanobenzyl 1H-imidazole-1-carboxylate as a tan solid (3.489 g, 100%).

¹H NMR (400 MHz, CDCl₃): δ 8.16 (t, J=1.1 Hz, 1H), 7.77-7.70 (m, 2H), 7.60-7.52 (m, 2H), 7.44 (t, J=1.5 Hz, 1H), 7.13-7.05 (m, 1H), 5.48 (s, 2H).

### 4-Cyanobenzyl benzylcarbamate (30.2)

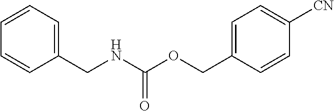 [SMILES: N#Cc1ccc(COC(=O)NCc2ccccc2)cc1]

4-Cyanobenzyl 1H-imidazole-1-carboxylate (0.756 g, 3.33 mmole) was dissolved in DMF (6 mL). Benzylamine (0.400 mL, 3.66 mmole) and N,N-diisopropylethylamine (0.630 mL, 3.62 mmole) were added and the solution was heated to 70° C. for 20 hours. The mixture was cooled to RT and diluted with ethyl acetate (25 mL). The mixture was washed with water (3×25 mL), dried over magnesium sulfate, filtered, and concentrated. The crude residue was purified by silica gel chromatography (10-40% ethyl acetate/hexane gradient) to provide 4-cyanobenzyl benzylcarbamate as a colorless oil (0.764 g, 86%).

¹H NMR (400 MHz, CDCl₃): δ 7.63 (d, J=7.9 Hz, 2H), 7.45 (d, J=7.9 Hz, 2H), 7.38-7.22 (m, 5H), 5.17 (s, 3H), 4.39 (d, J=6.0 Hz, 2H).

### 4-Carbamothioylbenzyl benzylcarbamate (30.3)

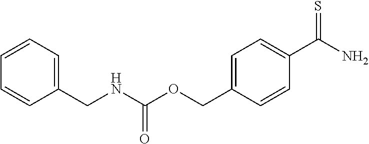 [SMILES: NC(=S)c1ccc(COC(=O)NCc2ccccc2)cc1]

4-Cyanobenzyl benzylcarbamate (0.764 g, 2.87 mmole) was dissolved in pyridine (3 mL). Triethylamine (0.450 mL, 3.21 mmole) and ammonium sulfide (40% aqueous solution, 0.590 mL, 3.45 mmole) were added and the mixture was heated to 50° C. for 4 hours, then was cooled to RT and concentrated. The residue was suspended in ethyl acetate (25 mL) and washed with 1N aq. HCl (2×25 mL). The slurry was filtered to provide a yellow solid. The filtrate was dried over magnesium sulfate, filtered, and concentrated to provide a yellow solid. The two solids were combined to provide 4-carbamothioylbenzyl benzylcarbamate as a yellow solid (0.707 g, 82%).

¹H NMR (400 MHz, DMSO-d₆): δ 9.87 (s, 1H), 9.49 (s, 1H), 7.93-7.81 (m, 3H), 7.37 (d, J=8.1 Hz, 2H), 7.35-7.28 (m, 2H), 7.25 (dt, J=8.3, 2.3 Hz, 3H), 5.08 (s, 2H), 4.21 (d, J=6.2 Hz, 2H).

### Ethyl 2-(4-(((benzylcarbamoyl)oxy)methyl)phenyl)thiazole-4-carboxylate (30.4)

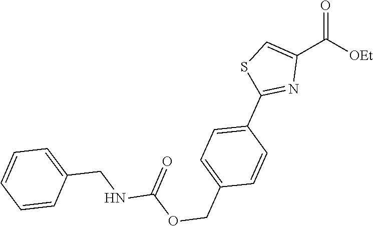 [SMILES: CCOC(=O)c1csc(-c2ccc(COC(=O)NCc3ccccc3)cc2)n1]

4-Carbamothioylbenzyl benzylcarbamate (0.707 g, 2.35 mmole) was dissolved in ethanol (12 mL). Ethyl bromopyruvate (0.360 mL, 2.87 mmole) was added and the mixture was heated to 80° C. for 3 hours, then was cooled to RT and concentrated. The residue was suspended in ethyl acetate (25 mL) and washed sequentially with sat. aq. sodium bicarbonate (2×25 mL) and water (1×25 mL). The combined aqueous washes were extracted with ethyl acetate (1×15 mL). The combined organics were dried over magnesium sulfate, filtered, and concentrated to provide ethyl 2-(4-(((benzylcarbamoyl)oxy)methyl)phenyl)thiazole-4-carboxylate as a yellow solid (0.935 g, 100%).

¹H NMR (400 MHz, CDCl₃): δ 8.15 (s, 1H), 7.99 (d, J=7.9 Hz, 2H), 7.44 (t, J=5.6 Hz, 2H), 7.38-7.27 (m, 5H), 5.18 (s, 3H), 4.45 (q, J=7.1 Hz, 2H), 4.41-4.34 (m, 2H), 1.43 (t, J=7.2 Hz, 3H).

### Methyl N-(2-(4-(((benzylcarbamoyl)oxy)methyl)phenyl)thiazole-4-carbonyl)-O-(tert-butyldimethylsilyl)-L-serinate (30.5)

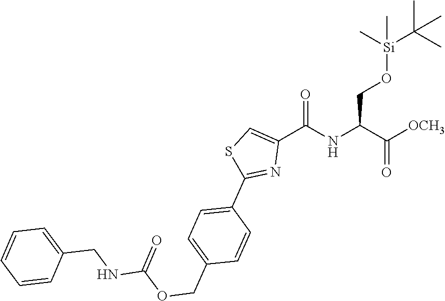 [SMILES: COC(=O)[C@H](CO[Si](C)(C)C(C)(C)C)NC(=O)c1csc(-c2ccc(COC(=O)NCc3ccccc3)cc2)n1]

Using the procedure described in Example 19.3, ethyl 2-(4-(((benzylcarbamoyl)oxy)methyl)phenyl)thiazole-4-carboxylate (0.935 g, 2.36 mmole) was converted to methyl N-(2-(4-(((benzylcarbamoyl)oxy)methyl)phenyl)thiazole-4-carbonyl)-O-(tert-butyldimethylsilyl)-L-serinate as a yellow oil (0.740 g, 54%).

¹H NMR (400 MHz, CDCl₃): δ 8.22 (d, J=8.7 Hz, 1H), 8.10 (s, 1H), 7.95 (d, J=7.9 Hz, 2H), 7.44 (d, J=7.9 Hz, 2H), 7.38-7.27 (m, 5H), 5.21 (s, 1H), 5.19 (s, 2H), 4.86 (dt, J=8.7, 3.0 Hz, 1H), 4.41 (d, J=6.0 Hz, 2H), 4.21 (dd, J=10.0, 2.6 Hz, 1H), 3.95 (dd, J=10.0, 3.3 Hz, 1H), 3.79 (s, 3H), 0.92 (s, 9H), 0.08 (s, 3H), 0.06 (s, 3H).

### Methyl O-acetyl-N—(N-(2-(4-(((benzylcarbamoyl)oxy)methyl)phenyl)thiazole-4-carbonyl)-O-(tert-butyldimethylsilyl)-L-seryl)-L-serinate (30.6)

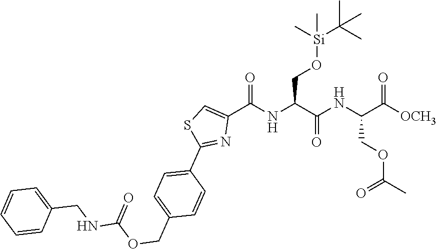 [SMILES: COC(=O)[C@H](COC(C)=O)NC(=O)[C@H](CO[Si](C)(C)C(C)(C)C)NC(=O)c1csc(-c2ccc(COC(=O)NCc3ccccc3)cc2)n1]

Using the procedure described in Example 15.6, methyl N-(2-(4-(((benzylcarbamoyl)oxy)methyl)phenyl)thiazole-4-carbonyl)-O-(tert-butyldimethylsilyl)-L-serinate (0.740 g, 1.27 mmole) was converted to methyl O-acetyl-N—(N-(2-(4-(((benzylcarbamoyl)oxy)methyl)phenyl)thiazole-4-carbonyl)-O-(tert-butyldimethylsilyl)-L-seryl)-L-serinate as a pale yellow gel (0.535 g, 59%).

¹H NMR (400 MHz, CDCl₃): δ 8.26 (d, J=7.1 Hz, 1H), 8.12 (d, J=2.5 Hz, 1H), 8.00-7.91 (m, 2H), 7.45 (t, J=8.6 Hz, 3H), 7.40-7.27 (m, 5H), 5.19 (s, 2H), 5.13 (s, 1H), 4.90 (tt, J=7.4, 3.8 Hz, 1H), 4.67 (td, J=7.4, 3.8 Hz, 1H), 4.49 (ddd, J=11.0, 7.0, 4.0 Hz, 1H), 4.45-4.37 (m, 2H), 4.34 (dd, J=11.4, 3.6 Hz, 1H), 4.23 (dt, J=9.9, 3.5 Hz, 1H), 3.85-3.78 (m, 1H), 3.77 (d, J=4.2 Hz, 3H), 2.02 (s, 3H), 0.96 (s, 9H), 0.17 (s, 3H), 0.16 (s, 3H).

### Methyl 2-(2-(2-(4-(((benzylcarbamoyl)oxy)methyl)phenyl)thiazole-4-carboxamido)acrylamido)acrylate (30)

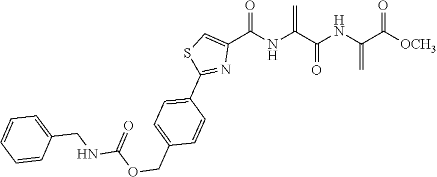 [SMILES: C=C(NC(=O)c1csc(-c2ccc(COC(=O)NCc3ccccc3)cc2)n1)C(=O)NC(=C)C(=O)OC]

Using the procedure described for Compound 15, methyl O-acetyl-N—(N-(2-(4-(((benzylcarbamoyl)oxy)methyl)phenyl)thiazole-4-carbonyl)-O-(tert-butyldimethylsilyl)-L-seryl)-L-serinate (0.535 g, 0.750 mmole) was converted to methyl 2-(2-(2-(4-(((benzylcarbamoyl)oxy)methyl)phenyl)thiazole-4-carboxamido)acrylamido)acrylate as a white solid (0.079 g, 20%).

¹H NMR (400 MHz, CDCl₃): δ 10.02 (s, 1H), 8.55 (s, 1H), 8.15 (s, 1H), 8.00 (d, J=8.0 Hz, 2H), 7.46 (d, J=7.9 Hz, 2H), 7.40-7.28 (m, 5H), 6.78 (d, J=2.2 Hz, 1H), 6.71 (s, 1H), 6.03 (d, J=1.3 Hz, 1H), 5.50 (t, J=1.9 Hz, 1H), 5.19 (s, 2H), 5.11 (s, 1H), 4.41 (d, J=6.0 Hz, 2H), 3.90 (s, 3H).

### Compound 31: Methyl (R)-2-(2-(2-(3-(((2-methoxyethoxy)carbonyl)amino)piperidin-1-yl)thiazole-4-carboxamido)acrylamido)acrylate

### Ethyl (R)-2-(3-((tert-butoxycarbonyl)amino)piperidin-1-yl)thiazole-4-carboxylate (31.1)

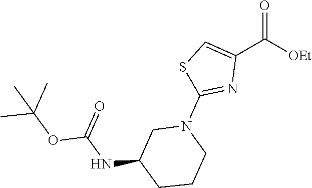 [SMILES: CCOC(=O)c1csc(N2CCC[C@@H](NC(=O)OC(C)(C)C)C2)n1]

Tert-butyl (R)-piperidin-3-ylcarbamate was dissolved in DMA. Ethyl 2-bromothiazole-4-carboxylate and triethylamine were added and the mixture was heated to 80° C. for 20 hours, then was cooled to RT and diluted with ethyl acetate (25 mL). The mixture was washed with water (3×25 mL) and the organics were dried over magnesium sulfate, filtered, and concentrated. The crude residue was purified by silica gel chromatography to provide ethyl (R)-2-(3-((tert-butoxycarbonyl)amino)piperidin-1-yl)thiazole-4-carboxylate as a white solid (1.399 g, 79%).

¹H NMR (400 MHz, CDCl3) δ 7.44 (s, 1H), 4.73 (s, 1H), 4.35 (q, J=7.1 Hz, 2H), 3.80 (s, 1H), 3.71 (d, J=11.9 Hz, 1H), 3.62 (s, 1H), 3.45 (s, 1H), 3.30 (d, J=9.1 Hz, 1H), 1.97-1.87 (m, 1H), 1.81 (ddt, J=10.8, 7.2, 3.5 Hz, 1H), 1.76-1.63 (m, 1H), 1.60 (s, 1H), 1.50 (d, J=7.7 Hz, 9H), 1.37 (t, J=7.1 Hz, 3H).

### Ethyl (R)-2-(3-(((2-methoxyethoxy)carbonyl)amino)piperidin-1-yl)thiazole-4-carboxylate (31.2)

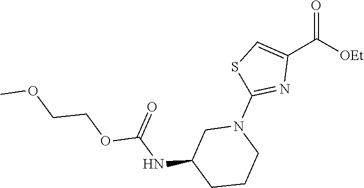 [SMILES: CCOC(=O)c1csc(N2CCC[C@@H](NC(=O)OCCOC)C2)n1]

Ethyl (R)-2-(3-((tert-butoxycarbonyl)amino)piperidin-1-yl)thiazole-4-carboxylate was dissolved in DCM·HCl (4M solution in 1,4-dioxane) was added and the mixture was stirred at RT for 2 hours and was concentrated. The residue was dissolved in DMF. 2-Methoxyethyl 1H-imidazole-1-carboxylate and N,N-diisopropylethylamine were added and the mixture was heated to 70° C. for 3 hours, then was cooled to RT and diluted with ethyl acetate (40 mL). The mixture was washed with water (3×25 mL) and the organics were dried over magnesium sulfate, filtered, and concentrated. The crude residue was purified by silica gel chromatography to provide ethyl (R)-2-(3-(((2-methoxyethoxy)carbonyl)amino)piperidin-1-yl)thiazole-4-carboxylate as a white solid (0.613 g, 44%).

¹H NMR (400 MHz, CDCl3) δ 7.45 (s, 1H), 4.99 (d, J=7.7 Hz, 1H), 4.35 (q, J=7.1 Hz, 2H), 4.23 (t, J=4.6 Hz, 2H), 3.93-3.78 (m, 1H), 3.77-3.67 (m, 1H), 3.67-3.61 (m, 1H), 3.61-3.53 (m, 2H), 3.46-3.42 (m, 1H), 3.39 (s, 3H), 3.32 (dd, J=12.7, 7.1 Hz, 1H), 1.90 (qd, J=7.3, 3.5 Hz, 1H), 1.83 (ddt, J=14.4, 7.3, 3.6 Hz, 1H), 1.76-1.66 (m, 1H), 1.66-1.55 (m, 1H), 1.37 (t, J=7.1 Hz, 3H).

### Methyl O-(tert-butyldimethylsilyl)-N-(2-((R)-3-(((2-methoxyethoxy)carbonyl)amino)piperidin-1-yl)thiazole-4-carbonyl)-L-serinate (31.3)

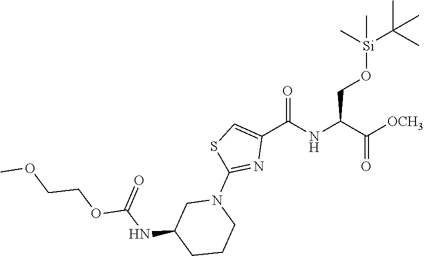 [SMILES: COCCOC(=O)N[C@@H]1CCCN(c2nc(C(=O)N[C@@H](CO[Si](C)(C)C(C)(C)C)C(=O)OC)cs2)C1]

Using the procedure described in Example 19.3, ethyl (R)-2-(3-(((2-methoxyethoxy)carbonyl)amino)piperidin-1-yl)thiazole-4-carboxylate (0.613 g, 1.72 mmole) was converted to methyl O-(tert-butyldimethylsilyl)-N-(2-((R)-3-(((2-methoxyethoxy)carbonyl)amino)piperidin-1-yl)thiazole-4-carbonyl)-L-serinate as a yellow oil (0.397 g, 42%).

¹H NMR (400 MHz, CDCl3) δ 7.93 (d, J=8.7 Hz, 1H), 7.39 (s, 1H), 4.99 (d, J=8.0 Hz, 1H), 4.83-4.73 (m, 1H), 4.23 (q, J=3.8 Hz, 2H), 4.16 (dd, J=10.0, 2.7 Hz, 1H), 3.93-3.83 (m, 2H), 3.76 (s, 3H), 3.68 (dd, J=12.6, 3.7 Hz, 1H), 3.59 (t, J=4.7 Hz, 2H), 3.54 (d, J=6.3 Hz, 1H), 3.44-3.37 (m, 1H), 3.39 (s, 3H), 3.29 (dd, J=12.7, 7.0 Hz, 1H), 1.92 (td, J=8.3, 3.8 Hz, 1H), 1.82 (dp, J=10.9, 3.6 Hz, 1H), 1.77-1.68 (m, 1H), 1.68-1.57 (m, 1H), 0.88 (s, 9H), 0.05 (s, 3H), 0.03 (s, 3H).

### Methyl O-acetyl-N—(O-(tert-butyldimethylsilyl)-N-(2-((R)-3-(((2-methoxyethoxy)carbonyl)amino)piperidin-1-yl)thiazole-4-carbonyl)-L-seryl)-L-serinate (31.4)

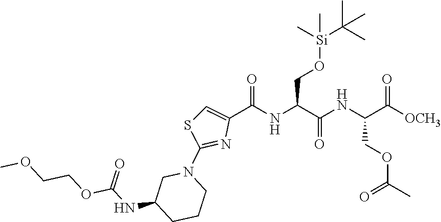 [SMILES: COCCOC(=O)N[C@@H]1CCCN(c2nc(C(=O)N[C@@H](CO[Si](C)(C)C(C)(C)C)C(=O)N[C@@H](COC(C)=O)C(=O)OC)cs2)C1]

Using the procedure described in Example 15.6, methyl O-(tert-butyldimethylsilyl)-N-(2-((R)-3-(((2-methoxyethoxy)carbonyl)amino)piperidin-1-yl)thiazole-4-carbonyl)-L-serinate (0.397 g, 0.729 mmole) was converted to methyl O-acetyl-N—(O-(tert-butyldimethylsilyl)-N-(2-((R)-3-(((2-methoxyethoxy)carbonyl)amino)piperidin-1-yl)thiazole-4-carbonyl)-L-seryl)-L-serinate as a pale yellow gel (0.367 g, 73%).

¹H NMR (400 MHz, CDCl3) δ 7.95 (d, J=7.2 Hz, 1H), 7.44 (d, J=7.8 Hz, 1H), 7.40 (s, 1H), 4.98 (t, J=10.1 Hz, 1H), 4.88 (dt, J=7.8, 3.8 Hz, 1H), 4.59 (td, J=7.3, 3.7 Hz, 1H), 4.47 (ddd, J=12.0, 8.1, 4.0 Hz, 1H), 4.31 (dd, J=11.4, 3.7 Hz, 1H), 4.23 (d, J=4.4 Hz, 2H), 4.18 (ddd, J=9.8, 3.7, 1.6 Hz, 1H), 3.91-3.81 (m, 1H), 3.76 (s, 3H), 3.75-3.70 (m, 1H), 3.62-3.52 (m, 3H), 3.44-3.39 (m, 1H), 3.39 (s, 3H), 3.31-3.14 (m, 1H), 2.05 (s, 3H), 1.98-1.87 (m, 1H), 1.83 (ddt, J=14.1, 7.0, 3.3 Hz, 1H), 1.72 (dq, J=9.6, 5.0, 4.5 Hz, 1H), 0.92 (s, 9H), 0.14 (s, 3H), 0.13 (s, 3H).

### Methyl (R)-2-(2-(2-(3-(((2-methoxyethoxy)carbonyl)amino)piperidin-1-yl)thiazole-4-carboxamido)acrylamido)acrylate (31)

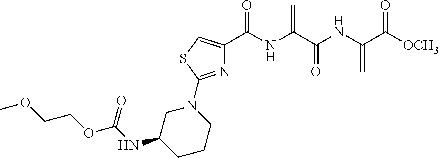 [SMILES: C=C(NC(=O)c1csc(N2CCC[C@@H](NC(=O)OCCOC)C2)n1)C(=O)NC(=C)C(=O)OC]

Using the procedure described for Compound 15, methyl O-acetyl-N—(O-(tert-butyldimethylsilyl)-N-(2-((R)-3-(((2-methoxyethoxy)carbonyl)amino)piperidin-1-yl)thiazole-4-carbonyl)-L-seryl)-L-serinate (0.361 g, 0.536 mmole) was converted to methyl (R)-2-(2-(2-(3-(((2-methoxyethoxy)carbonyl)amino)piperidin-1-yl)thiazole-4-carboxamido)acrylamido)acrylate as a colorless gel (0.116 g, 45%).

¹H NMR (400 MHz, CDCl3) δ 9.69 (s, 1H), 8.51 (s, 1H), 7.44 (s, 1H), 6.71 (d, J=2.1 Hz, 1H), 6.67 (s, 1H), 6.00 (d, J=1.3 Hz, 1H), 5.44 (t, J=1.8 Hz, 1H), 5.00 (d, J=7.9 Hz, 1H), 4.24 (dd, J=6.9, 2.6 Hz, 2H), 3.92-3.83 (m, 1H), 3.89 (s, 3H), 3.67 (d, J=15.8 Hz, 1H), 3.58 (t, J=4.6 Hz, 2H), 3.44 (s, 1H), 3.39 (s, 3H), 3.35 (t, J=6.3 Hz, 1H), 1.89 (d, J=3.0 Hz, 1H), 1.84 (dt, J=7.1, 3.1 Hz, 1H), 1.78-1.62 (m, 3H).

### Compound 32: Methyl 2-(2-(2-(4-((((3-methoxypropyl)carbamoyl)oxy)methyl)piperidin-1-yl)thiazole-4-carboxamido)acrylamido)acrylate

### Tert-butyl 4-(((1H-imidazole-1-carbonyl)oxy)methyl)piperidine-1-carboxylate (32.1)

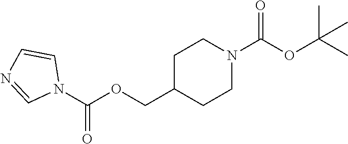 [SMILES: CC(C)(C)OC(=O)N1CCC(COC(=O)n2ccnc2)CC1]

Using the procedure described in Example 27.1, tert-butyl 4-(hydroxymethyl)piperidine-1-carboxylate (2.007 g, 9.32 mmole) was converted to tert-butyl 4-(((1H-imidazole-1-carbonyl)oxy)methyl)piperidine-1-carboxylate (3.389 g) as a pale yellow oil.

¹H NMR (400 MHz, CDCl3) δ 8.14 (t, J=1.1 Hz, 1H), 7.42 (t, J=1.5 Hz, 1H), 7.08 (dd, J=1.6, 0.9 Hz, 1H), 4.29 (d, J=6.6 Hz, 2H), 4.24-4.13 (m, 2H), 2.74 (t, J=12.8 Hz, 2H), 2.03-1.93 (m, 1H), 1.81-1.70 (m, 2H), 1.46 (s, 9H), 1.34-1.26 (m, 2H).

### Tert-butyl 4-((((3-methoxypropyl)carbamoyl)oxy)methyl)piperidine-1-carboxylate (32.2)

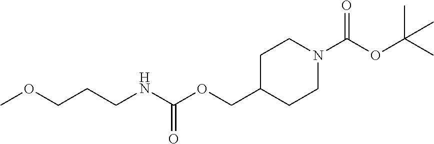 [SMILES: COCCCNC(=O)OCC1CCN(C(=O)OC(C)(C)C)CC1]

Tert-butyl 4-(((1H-imidazole-1-carbonyl)oxy)methyl)piperidine-1-carboxylate (0.994 g, 3.21 mmole) was dissolved in DMF (6 mL). 3-Methoxypropan-1-amine (0.365 mL, 3.58 mmole) and N,N-diisopropylethylamine (0.680 mL, 3.90 mmole) were added and the mixture was heated to 70° C. for 18 hours, then was cooled to RT and diluted with ethyl acetate (25 mL). The mixture was washed with water (3×25 mL) and the organics were dried over magnesium sulfate, filtered, and concentrated. The crude residue was purified by silica gel chromatography to provide tert-butyl 4-((((3-methoxypropyl)carbamoyl)oxy)methyl)piperidine-1-carboxylate as a colorless oil (0.779 g, 73%).

¹H NMR (400 MHz, CDCl3) δ 5.10 (s, 1H), 4.17-4.05 (m, 2H), 3.91 (d, J=6.5 Hz, 2H), 3.46 (t, J=5.8 Hz, 2H), 3.34 (s, 3H), 3.28 (q, J=6.3 Hz, 2H), 2.77-2.61 (m, 2H), 1.83-1.73 (m, 3H), 1.70 (s, 2H), 1.45 (s, 9H), 1.22-1.10 (m, 2H).

### Ethyl 2-(4-((((3-methoxypropyl)carbamoyl)oxy)methyl)piperidin-1-yl)thiazole-4-carboxylate (32.3)

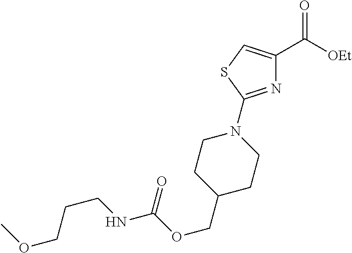 [SMILES: CCOC(=O)c1csc(N2CCC(COC(=O)NCCCOC)CC2)n1]

Tert-butyl 4-((((3-methoxypropyl)carbamoyl)oxy)methyl)piperidine-1-carboxylate (0.779 g, 2.36 mmole) was dissolved in DCM (5 mL). HCl (4M solution in 1,4-dioxane, 2.40 mL, 9.60 mmole) was added and the mixture was stirred at RT for 2 hours, then was concentrated. The residue was dissolved in DMA (3 mL). Ethyl 2-bromothiazole-4-carboxylate (0.564 g, 2.39 mmole) and triethylamine (0.660 mL, 4.71 mmole) were added and the mixture was heated to 80° C. for 20 hours, then was cooled to RT and diluted with ethyl acetate (25 mL). The mixture was washed with water (3×25 mL) and the organics were dried over magnesium sulfate, filtered, and concentrated. The crude residue was purified by silica gel chromatography to provide ethyl 2-(4-((((3-methoxypropyl)carbamoyl)oxy)methyl)piperidin-1-yl)thiazole-4-carboxylate as a pale tan solid (0.460 g, 51%).

¹H NMR (400 MHz, CDCl3) δ 7.42 (s, 1H), 5.08 (s, 1H), 4.35 (q, J=7.1 Hz, 2H), 4.07 (dt, J=12.7, 3.4 Hz, 2H), 3.95 (d, J=6.2 Hz, 2H), 3.46 (t, J=5.8 Hz, 2H), 3.34 (s, 3H), 3.29 (q, J=6.2 Hz, 2H), 3.03 (td, J=12.6, 2.8 Hz, 2H), 1.95-1.67 (m, 5H), 1.37 (t, J=7.1 Hz, 5H).

### Methyl O-(tert-butyldimethylsilyl)-N-(2-(4-((((3-methoxypropyl)carbamoyl)oxy)methyl)piperidin-1-yl)thiazole-4-carbonyl)-L-serinate (32.4)

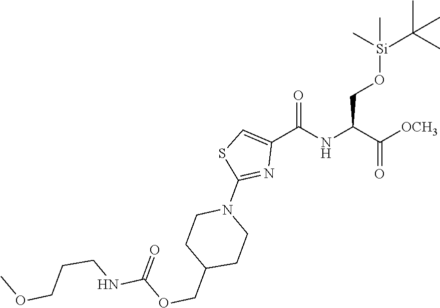 [SMILES: COCCCNC(=O)OCC1CCN(c2nc(C(=O)N[C@@H](CO[Si](C)(C)C(C)(C)C)C(=O)OC)cs2)CC1]

Using the procedure described in Example 19.3, ethyl 2-(4-((((3-methoxypropyl)carbamoyl)oxy)methyl)piperidin-1-yl)thiazole-4-carboxylate (0.460 g, 1.19 mmole) was converted to methyl O-(tert-butyldimethylsilyl)-N-(2-(4-((((3-methoxypropyl)carbamoyl)oxy)methyl)piperidin-1-yl)thiazole-4-carbonyl)-L-serinate as a pale yellow oil (0.447 g, 66%).

¹H NMR (400 MHz, CDCl3) δ 7.97 (d, J=8.8 Hz, 1H), 7.36 (s, 1H), 5.08 (s, 1H), 4.82-4.74 (m, 1H), 4.16 (dd, J=10.0, 2.6 Hz, 1H), 4.02 (d, J=12.8 Hz, 2H), 3.96 (d, J=6.2 Hz, 2H), 3.88 (dd, J=10.0, 3.4 Hz, 1H), 3.76 (s, 3H), 3.47 (t, J=5.8 Hz, 2H), 3.34 (s, 3H), 3.29 (t, J=6.3 Hz, 2H), 3.01 (tdd, J=12.4, 5.5, 2.8 Hz, 2H), 1.96-1.86 (m, 1H), 1.85-1.74 (m, 4H), 1.47-1.31 (m, 2H), 0.88 (s, 9H), 0.06 (s, 3H), 0.03 (s, 3H).

### Methyl O-acetyl-N—(O-(tert-butyldimethylsilyl)-N-(2-(4-((((3-methoxypropyl)carbamoyl)oxy)methyl)piperidin-1-yl)thiazole-4-carbonyl)-L-seryl)-L-serinate (32.5)

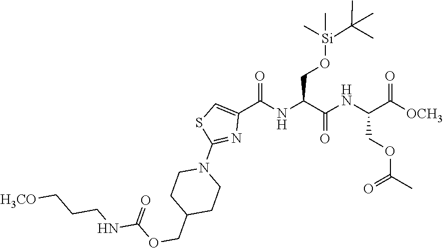 [SMILES: COCCCNC(=O)OCC1CCN(c2nc(C(=O)N[C@@H](CO[Si](C)(C)C(C)(C)C)C(=O)N[C@@H](COC(C)=O)C(=O)OC)cs2)CC1]

Using the procedure described in Example 15.6, methyl O-(tert-butyldimethylsilyl)-N-(2-(4-((((3-methoxypropyl)carbamoyl)oxy)methyl)piperidin-1-yl)thiazole-4-carbonyl)-L-serinate (0.447 g, 0.780 mmole) was converted to methyl O-acetyl-N—(O-(tert-butyldimethylsilyl)-N-(2-(4-((((3-methoxypropyl)carbamoyl)oxy)methyl)piperidin-1-yl)thiazole-4-carbonyl)-L-seryl)-L-serinate as a pale yellow oil (0.366 g, 67%).

¹H NMR (400 MHz, CDCl3) δ 8.00 (d, J=7.1 Hz, 1H), 7.43 (d, J=7.8 Hz, 1H), 7.38 (s, 1H), 5.09 (s, 1H), 4.88 (dt, J=7.7, 3.8 Hz, 1H), 4.58 (ddd, J=7.2, 5.8, 3.6 Hz, 1H), 4.47 (dt, J=11.4, 4.2 Hz, 1H), 4.31 (dd, J=11.4, 3.7 Hz, 1H), 4.19 (ddd, J=9.8, 3.6, 2.2 Hz, 1H), 4.02 (d, J=12.9 Hz, 2H), 3.96 (d, J=6.2 Hz, 2H), 3.76 (s, 3H), 3.75-3.70 (m, 1H), 3.47 (t, J=5.8 Hz, 2H), 3.34 (s, 3H), 3.29 (t, J=6.2 Hz, 2H), 3.00 (ddd, J=15.5, 7.9, 3.2 Hz, 2H), 2.05 (s, 3H), 1.87 (d, J=10.7 Hz, 1H), 1.85-1.73 (m, 4H), 1.47-1.32 (m, 2H), 0.93 (s, 9H), 0.14 (s, 3H), 0.13 (s, 3H).

### Methyl 2-(2-(2-(4-((((3-methoxypropyl)carbamoyl)oxy)methyl)piperidin-1-yl)thiazole-4-carboxamido)acrylamido)acrylate (32)

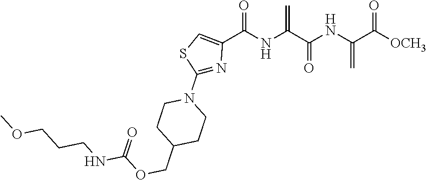 [SMILES: C=C(NC(=O)c1csc(N2CCC(COC(=O)NCCCOC)CC2)n1)C(=O)NC(=C)C(=O)OC]

Using the procedure described for Compound 15, methyl O-acetyl-N—(O-(tert-butyldimethylsilyl)-N-(2-(4-((((3-methoxypropyl)carbamoyl)oxy)methyl)piperidin-1-yl)thiazole-4-carbonyl)-L-seryl)-L-serinate (0.366 g, 0.521 mmole) was converted to methyl 2-(2-(2-(4-((((3-methoxypropyl)carbamoyl)oxy)methyl)piperidin-1-yl)thiazole-4-carboxamido)acrylamido)acrylate as a white solid (0.098 g, 37%).

¹H NMR (400 MHz, CDCl3) δ 9.73 (s, 1H), 8.51 (s, 1H), 7.41 (s, 1H), 6.71 (d, J=2.1 Hz, 1H), 6.66 (s, 1H), 6.00 (d, J=1.3 Hz, 1H), 5.43 (t, J=1.8 Hz, 1H), 5.07 (s, 1H), 4.05 (dd, J=13.0, 3.7 Hz, 2H), 3.97 (d, J=6.2 Hz, 2H), 3.89 (s, 3H), 3.47 (t, J=5.8 Hz, 2H), 3.34 (s, 3H), 3.30 (q, J=6.2 Hz, 2H), 3.03 (td, J=12.7, 2.8 Hz, 2H), 1.90 (d, J=6.3 Hz, 1H), 1.84 (d, J=13.3 Hz, 2H), 1.79 (q, J=6.2 Hz, 2H), 1.47-1.32 (m, 2H).

### Compound 33: Methyl 2-(2-(2-(4-(((2-methoxyethoxy)carbonyl)amino)piperidin-1-yl)thiazole-4-carboxamido)acrylamido)acrylate

### Ethyl 2-(4-(((2-methoxyethoxy)carbonyl)amino)piperidin-1-yl)thiazole-4-carboxylate (33.1)

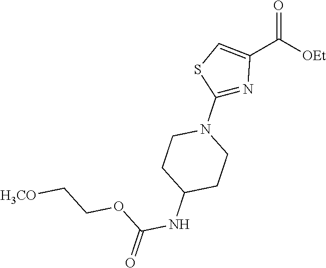 [SMILES: CCOC(=O)c1csc(N2CCC(NC(=O)OCCOC)CC2)n1]

Using the procedure described in Example 27.2, ethyl 2-(4-((tert-butoxycarbonyl)amino)piperidin-1-yl)thiazole-4-carboxylate (Example 12.1, 0.956 g, 2.69 mmole) was converted to ethyl 2-(4-(((2-methoxyethoxy)carbonyl)amino)piperidin-1-yl)thiazole-4-carboxylate (0.512 g, 53%) as a white solid.

¹H NMR (400 MHz, CDCl3) δ 7.44 (s, 1H), 4.76 (d, J=7.5 Hz, 1H), 4.35 (q, J=7.1 Hz, 2H), 4.23 (t, J=4.5 Hz, 2H), 4.04-3.94 (m, 2H), 3.73 (td, J=10.9, 9.2, 5.1 Hz, 1H), 3.62-3.55 (m, 2H), 3.40 (s, 3H), 3.17 (ddd, J=13.3, 11.5, 3.0 Hz, 2H), 2.06 (ddt, J=12.6, 5.0, 2.3 Hz, 2H), 1.51 (dtd, J=12.8, 11.2, 4.3 Hz, 2H), 1.37 (t, J=7.1 Hz, 3H).

### Methyl O-(tert-butyldimethylsilyl)-N-(2-(4-(((2-methoxyethoxy)carbonyl)amino)piperidin-1-yl)thiazole-4-carbonyl)-L-serinate (33.2)

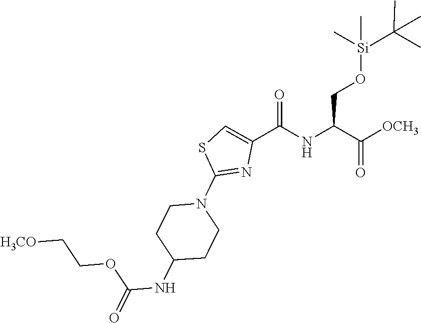 [SMILES: COCCOC(=O)NC1CCN(c2nc(C(=O)N[C@@H](CO[Si](C)(C)C(C)(C)C)C(=O)OC)cs2)CC1]

Using the procedure described in Example 19.3, ethyl 2-(4-(((2-methoxyethoxy)carbonyl)amino)piperidin-1-yl)thiazole-4-carboxylate (0.512 g, 1.43 mmole) was converted to methyl O-(tert-butyldimethylsilyl)-N-(2-(4-(((2-methoxyethoxy)carbonyl)amino)piperidin-1-yl)thiazole-4-carbonyl)-L-serinate as a colorless oil (0.398 g, 51%).

¹H NMR (400 MHz, CDCl3) δ 7.95 (d, J=8.8 Hz, 1H), 7.38 (s, 1H), 4.82-4.74 (m, 2H), 4.27-4.20 (m, 2H), 4.20-4.14 (m, 1H), 4.00-3.91 (m, 2H), 3.88 (dd, J=10.0, 3.4 Hz, 1H), 3.76 (s, 4H), 3.63-3.55 (m, 2H), 3.40 (s, 3H), 3.15 (dddd, J=13.3, 11.5, 7.2, 3.0 Hz, 2H), 2.09-2.00 (m, 2H), 1.59-1.45 (m, 2H), 0.88 (s, 9H), 0.05 (s, 3H), 0.03 (s, 3H).

### Methyl O-acetyl-N—(O-(tert-butyldimethylsilyl)-N-(2-(4-(((2-methoxyethoxy)carbonyl)amino)piperidin-1-yl)thiazole-4-carbonyl)-L-seryl)-L-serinate (33.3)

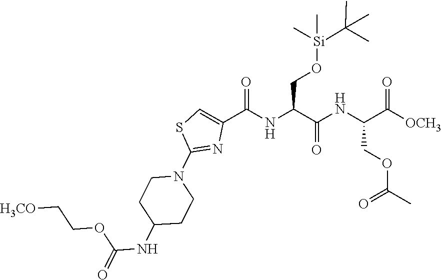 [SMILES: COCCOC(=O)NC1CCN(c2nc(C(=O)N[C@@H](CO[Si](C)(C)C(C)(C)C)C(=O)N[C@@H](COC(C)=O)C(=O)OC)cs2)CC1]

Using the procedure described in Example 15.6, methyl O-(tert-butyldimethylsilyl)-N-(2-(4-(((2-methoxyethoxy)carbonyl)amino)piperidin-1-yl)thiazole-4-carbonyl)-L-serinate (0.398 g, 0.731 mmole) was converted to methyl O-acetyl-N—(O-(tert-butyldimethylsilyl)-N-(2-(4-(((2-methoxyethoxy)carbonyl)amino)piperidin-1-yl)thiazole-4-carbonyl)-L-seryl)-L-serinate as a pale yellow oil (0.366 g, 74%).

¹H NMR (400 MHz, CDCl3) δ 7.98 (d, J=7.1 Hz, 1H), 7.43 (d, J=7.8 Hz, 1H), 7.39 (s, 1H), 4.87 (dt, J=7.7, 3.8 Hz, 1H), 4.77 (d, J=7.8 Hz, 1H), 4.63-4.54 (m, 1H), 4.47 (ddd, J=11.3, 9.5, 4.0 Hz, 1H), 4.31 (dd, J=11.4, 3.7 Hz, 1H), 4.27-4.21 (m, 2H), 4.21-4.17 (m, 1H), 3.94 (d, J=10.4 Hz, 2H), 3.80-3.69 (m, 2H), 3.76 (s, 3H), 3.59 (dd, J=5.5, 3.6 Hz, 2H), 3.40 (s, 3H), 3.22-3.09 (m, 2H), 2.11-2.05 (m, 2H), 2.05 (s, 3H), 1.50 (dd, J=14.1, 9.5 Hz, 2H), 0.92 (s, 9H), 0.14 (s, 3H), 0.13 (s, 3H).

### Methyl 2-(2-(2-(4-(((2-methoxyethoxy)carbonyl)amino)piperidin-1-yl)thiazole-4-carboxamido)acrylamido)acrylate (33)

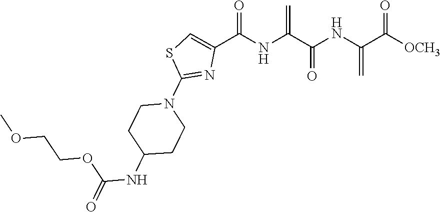 [SMILES: C=C(NC(=O)c1csc(N2CCC(NC(=O)OCCOC)CC2)n1)C(=O)NC(=C)C(=O)OC]

Using the procedure described for Compound 15, methyl O-acetyl-N—(O-(tert-butyldimethylsilyl)-N-(2-(4-(((2-methoxyethoxy)carbonyl)amino)piperidin-1-yl)thiazole-4-carbonyl)-L-seryl)-L-serinate (0.366 g, 0.543 mmole) was converted to methyl 2-(2-(2-(4-(((2-methoxyethoxy)carbonyl)amino)piperidin-1-yl)thiazole-4-carboxamido)acrylamido)acrylate as a white solid (0.124 g, 47%).

¹H NMR (400 MHz, CDCl3) δ 9.71 (s, 1H), 8.51 (s, 1H), 7.43 (s, 1H), 6.71 (d, J=2.1 Hz, 1H), 6.66 (s, 1H), 6.00 (d, J=1.3 Hz, 1H), 5.43 (t, J=1.9 Hz, 1H), 4.78 (d, J=7.8 Hz, 1H), 4.24 (t, J=4.6 Hz, 2H), 3.98 (dt, J=13.6, 3.9 Hz, 2H), 3.89 (s, 3H), 3.83-3.69 (m, 1H), 3.64-3.56 (m, 2H), 3.40 (s, 3H), 3.18 (ddd, J=13.2, 11.5, 3.0 Hz, 2H), 2.15-2.05 (m, 2H), 1.53 (dtd, J=12.8, 11.1, 4.3 Hz, 2H).

### Compound 34: Methyl 2-(2-(2-(4-(2-(2-methoxyethoxy)acetamido)piperidin-1-yl)thiazole-4-carboxamido)acrylamido)acrylate

### Methyl (2-(4-((tert-butoxycarbonyl)amino)piperidin-1-yl)thiazole-4-carbonyl)-L-serinate (34.1)

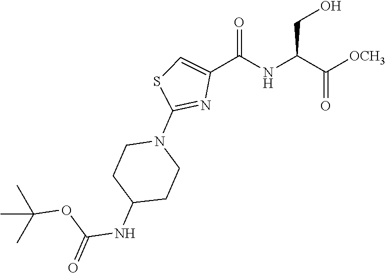 [SMILES: COC(=O)[C@H](CO)NC(=O)c1csc(N2CCC(NC(=O)OC(C)(C)C)CC2)n1]

Ethyl 2-(4-((tert-butoxycarbonyl)amino)piperidin-1-yl)thiazole-4-carboxylate (Example 12.1, 1.526 g, 4.29 mmole) was dissolved in 4/1/1 THF/methanol/water (8 mL). Lithium hydroxide monohydrate (0.356 g, 8.48 mmole) was added and the mixture was stirred at RT for 4 hours. Water (25 mL) was added and the solution was treated with 1N aq. HCl to pH=4. The mixture was extracted with ethyl acetate (3×15 mL) and the combined organics were dried over magnesium sulfate, filtered, and concentrated. The residue was suspended in DCM (8 mL). L-Serine methyl ester hydrochloride (0.795 g, 5.11 mmole) was added followed by N,N-diisopropylethylamine (1.50 mL, 8.61 mmole) and pyBOP (2.673 g, 5.14 mmole). The resulting solution was stirred at RT for 18 hours and water (25 mL) was added. The two layers were separated and the aqueous layer was extracted with DCM (3×10 mL). The combined organics were dried over magnesium sulfate, filtered, and concentrated. The crude residue was purified by silica gel chromatography to provide methyl (2-(4-((tert-butoxycarbonyl)amino)piperidin-1-yl)thiazole-4-carbonyl)-L-serinate as a white solid (1.648 g, 90%).

¹H NMR (400 MHz, CDCl3) δ 7.99 (d, J=7.5 Hz, 1H), 7.38 (s, 1H), 4.79 (dt, J=7.6, 3.8 Hz, 1H), 4.59 (d, J=7.9 Hz, 1H), 4.08-3.99 (m, 2H), 3.94 (ddd, J=13.0, 5.5, 2.9 Hz, 2H), 3.81 (s, 3H), 3.65 (d, J=25.0 Hz, 1H), 3.13 (ddt, J=13.6, 11.2, 2.6 Hz, 2H), 2.04 (dd, J=12.9, 3.7 Hz, 2H), 1.57-1.47 (m, 2H), 1.46 (s, 9H).

### Methyl O-(tert-butyldimethylsilyl)-N-(2-(4-(2-(2-methoxyethoxy)acetamido)piperidin-1-yl)thiazole-4-carbonyl)-L-serinate (34.2)

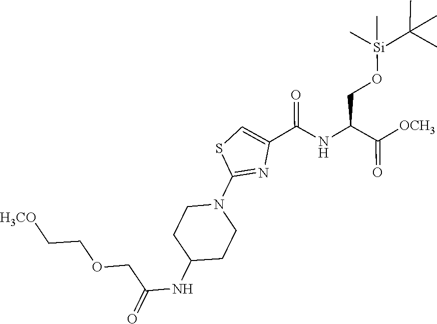 [SMILES: COCCOCC(=O)NC1CCN(c2nc(C(=O)N[C@@H](CO[Si](C)(C)C(C)(C)C)C(=O)OC)cs2)CC1]

Methyl (2-(4-((tert-butoxycarbonyl)amino)piperidin-1-yl)thiazole-4-carbonyl)-L-serinate (0.506 g, 1.18 mmole) was dissolved in DCM (2.5 mL). HCl (4M solution in 1,4-dioxane, 1.20 mL, 4.80 mmole) was added and the mixture was stirred at RT for 5 hours, then was concentrated. A solution of 2-(2-methoxyethoxy)acetic acid (0.192 g, 1.43 mmole) in DCM (2.5 mL) was added to the residue, followed by N,N-diisopropylethylamine (0.410 mL, 2.35 mmole), HOBt·H2O (0.221 g, 1.44 mmole), and EDC·HCl (0.267 g, 1.39 mmole). The mixture was stirred at RT for 2½ days and water (25 mL) was added. The mixture was extracted with DCM (3×10 mL) and the combined organics were dried over magnesium sulfate, filtered, and concentrated. The residue was dissolved in DCM (2.5 mL) and imidazole (0.088 g, 1.29 mmole) and tert-butyldimethylchlorosilane (0.192 g, 1.27 mmole) were added. The mixture was stirred at RT for 18 hours and water (25 mL) was added. The mixture was extracted with DCM (3×10 mL) and the combined organics were dried over magnesium sulfate, filtered, and concentrated. The crude residue was purified by silica gel chromatography to provide methyl O-(tert-butyldimethylsilyl)-N-(2-(4-(2-(2-methoxyethoxy)acetamido)piperidin-1-yl)thiazole-4-carbonyl)-L-serinate as a thick, colorless oil (0.361 g, 55%).

¹H NMR (400 MHz, CDCl3) δ 7.96 (d, J=8.8 Hz, 1H), 7.39 (s, 1H), 7.13 (d, J=8.0 Hz, 1H), 4.78 (ddd, J=8.8, 3.4, 2.6 Hz, 1H), 4.16 (dd, J=10.0, 2.6 Hz, 1H), 4.12-4.03 (m, 1H), 4.00 (s, 3H), 3.97-3.91 (m, 1H), 3.88 (dd, J=10.0, 3.4 Hz, 1H), 3.76 (s, 3H), 3.71-3.64 (m, 2H), 3.59-3.52 (m, 2H), 3.39 (s, 3H), 3.18 (dddd, J=13.2, 11.7, 8.7, 3.0 Hz, 2H), 2.09-1.98 (m, 2H), 1.68-1.51 (m, 2H), 0.88 (s, 9H), 0.05 (s, 3H), 0.03 (s, 3H).

### Methyl O-acetyl-N—(O-(tert-butyldimethylsilyl)-N-(2-(4-(2-(2-methoxyethoxy)acetamido)piperidin-1-yl)thiazole-4-carbonyl)-L-seryl)-L-serinate (34.3)

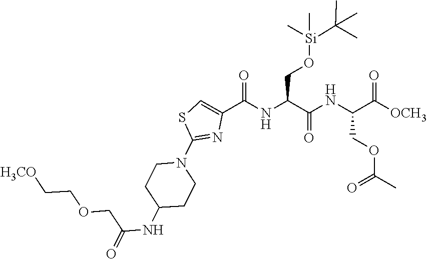 [SMILES: COCCOCC(=O)NC1CCN(c2nc(C(=O)N[C@@H](CO[Si](C)(C)C(C)(C)C)C(=O)N[C@@H](COC(C)=O)C(=O)OC)cs2)CC1]

Using the procedure described in Example 15.6, methyl O-(tert-butyldimethylsilyl)-N-(2-(4-(2-(2-methoxyethoxy)acetamido)piperidin-1-yl)thiazole-4-carbonyl)-L-serinate (0.361 g, 0.646 mmole) was converted to methyl O-acetyl-N—(O-(tert-butyldimethylsilyl)-N-(2-(4-(2-(2-methoxyethoxy)acetamido)piperidin-1-yl)thiazole-4-carbonyl)-L-seryl)-L-serinate as a colorless gel (0.267 g, 60%).

¹H NMR (400 MHz, CDCl3) δ 7.99 (d, J=7.1 Hz, 1H), 7.44 (d, J=7.8 Hz, 1H), 7.40 (d, J=2.6 Hz, 1H), 7.13 (d, J=8.0 Hz, 1H), 4.87 (dq, J=8.3, 4.2 Hz, 1H), 4.58 (tdd, J=7.0, 3.6, 2.1 Hz, 1H), 4.47 (ddd, J=11.4, 9.0, 4.0 Hz, 1H), 4.31 (dd, J=11.4, 3.7 Hz, 1H), 4.19 (ddd, J=9.8, 3.6, 1.2 Hz, 1H), 4.09-4.02 (m, 1H), 4.02-3.91 (m, 2H), 3.99 (s, 2H), 3.76 (s, 3H), 3.75-3.71 (m, 1H), 3.71-3.65 (m, 2H), 3.60-3.51 (m, 2H), 3.39 (s, 3H), 3.18 (dddd, J=13.0, 11.5, 4.7, 2.9 Hz, 2H), 2.09-2.03 (m, 2H), 2.02 (s, 3H), 1.63-1.51 (m, 2H), 0.92 (d, J=1.0 Hz, 9H), 0.14 (s, 3H), 0.13 (s, 3H).

### Methyl 2-(2-(2-(4-(2-(2-methoxyethoxy)acetamido)piperidin-1-yl)thiazole-4-carboxamido)acrylamido)acrylate (34)

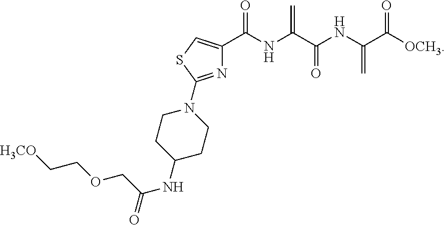 [SMILES: C=C(NC(=O)c1csc(N2CCC(NC(=O)COCCOC)CC2)n1)C(=O)NC(=C)C(=O)OC]

Using the procedure described for Compound 15, methyl O-acetyl-N—(O-(tert-butyldimethylsilyl)-N-(2-(4-(((2-methoxyethoxy)carbonyl)amino)piperidin-1-yl)thiazole-4-carbonyl)-L-seryl)-L-serinate (0.366 g, 0.543 mmole) was converted to methyl 2-(2-(2-(4-(((2-methoxyethoxy)carbonyl)amino)piperidin-1-yl)thiazole-4-carboxamido)acrylamido)acrylate as a white solid (0.124 g, 47%).

¹H NMR (400 MHz, CDCl3) δ 9.72 (s, 1H), 8.51 (s, 1H), 7.43 (s, 1H), 7.13 (d, J=8.0 Hz, 1H), 6.72 (d, J=2.1 Hz, 1H), 6.66 (s, 1H), 6.01 (d, J=1.3 Hz, 1H), 5.44 (t, J=1.8 Hz, 1H), 4.13-4.04 (m, 1H), 4.04-3.94 (m, 2H), 3.99 (s, 2H), 3.89 (s, 3H), 3.71-3.63 (m, 2H), 3.59-3.51 (m, 2H), 3.39 (s, 3H), 3.27-3.15 (m, 2H), 2.12-2.01 (m, 2H), 1.63-1.51 (m, 2H).

### Compound 35: Methyl 2-(2-(2-(4-((2-(2-methoxyethoxy)acetamido)methyl)phenyl)thiazole-4-carboxamido)acrylamido)acrylate

### Tert-butyl (4-cyanobenzyl)carbamate (35.1)

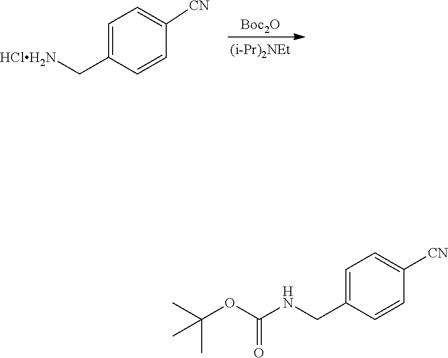

4-(Aminomethyl)benzonitrile hydrochloride (3.013 g, 17.9 mmole) was suspended in DCM (36 mL). Di-tert-butyl dicarbonate (4.100 g, 18.8 mmole) was added followed by N,N-diisopropylethylamine (6.20 mL, 35.6 mmole) and the mixture was stirred at RT for 18 hours. Water (50 mL) was added and the two layers were separated. The aqueous layer was extracted with DCM (2×15 mL) and the combined organics were dried over magnesium sulfate, filtered, and concentrated to provide tert-butyl (4-cyanobenzyl)carbamate as a white solid (4.901 g).

¹H NMR (400 MHz, CDCl₃): δ 7.66-7.58 (m, 2H), 7.39 (d, J=8.2 Hz, 2H), 5.03 (s, 1H), 4.37 (d, J=6.3 Hz, 2H), 1.46 (s, 9H).

### Tert-butyl (4-carbamothioylbenzyl)carbamate (35.2)

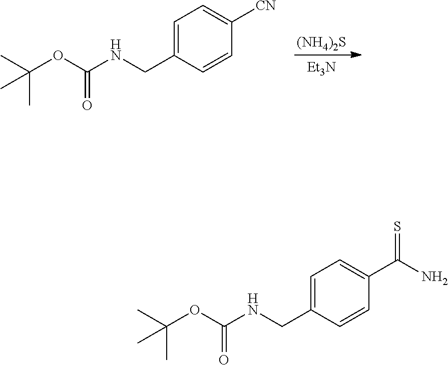 [SMILES: CC(C)(C)OC(=O)NCc1ccc(C#N)cc1 ; CC(C)(C)OC(=O)NCc1ccc(C(N)=S)cc1]

Tert-butyl (4-cyanobenzyl)carbamate (4.901 g, 21.1 mmole) was dissolved in pyridine (21 mL). Triethylamine (3.30 mL, 23.5 mmole) and ammonium sulfide (40% aqueous solution, 4.40 mL, 25.8 mmole) were added and the mixture was heated to 50° C. for 4½ hours, then was cooled to RT and concentrated. The residue was dissolved in ethyl acetate (50 mL) and the mixture was washed sequentially with 1N aq. HCl (2×25 mL) and sat. aq. sodium chloride (1×25 mL). The organics were dried over magnesium sulfate, filtered, and concentrated to provide tert-butyl (4-carbamothioylbenzyl)carbamate as a yellow solid (5.376 g).

1H NMR (400 MHz, DMSO) δ 9.81 (s, 1H), 9.44 (s, 1H), 7.89-7.82 (m, 2H), 7.44 (q, J=6.5 Hz, 1H), 7.25 (d, J=8.3 Hz, 2H), 4.15 (d, J=6.2 Hz, 2H), 1.39 (s, 9H).

### Ethyl 2-(4-(((tert-butoxycarbonyl)amino)methyl)phenyl)thiazole-4-carboxylate (35.3)

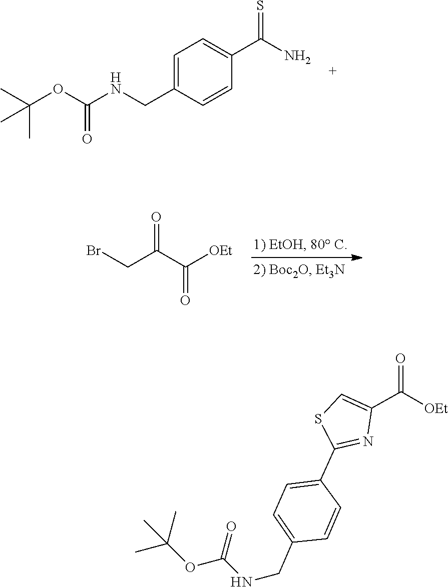 [SMILES: CC(C)(C)OC(=O)NCc1ccc(C(N)=S)cc1 ; CCOC(=O)C(=O)CBr ; CCOC(=O)c1csc(-c2ccc(CNC(=O)OC(C)(C)C)cc2)n1]

Tert-butyl (4-carbamothioylbenzyl)carbamate (5.376 g, 20.1 mmole) was dissolved in ethanol (40 mL). Ethyl bromopyruvate (2.80 mL, 22.3 mmole) was added and the mixture was heated to 80° C. for 18 hours, then was cooled to RT and concentrated. The residue was suspended in DCM (40 mL) and triethylamine (5.70 mL, 40.7 mmole) and di-tert-butyl dicarbonate (4.857 g, 22.3 mmole) were added. The mixture was stirred at RT for 18 hours and water (100 mL) was added. The two layers were separated and the aqueous layer was extracted with DCM (2×20 mL). The combined organics were dried over magnesium sulfate, filtered, and concentrated. The crude residue was purified by silica gel chromatography to provide ethyl 2-(4-(((tert-butoxycarbonyl)amino)methyl)phenyl)thiazole-4-carboxylate as an orange solid (5.003 g, 69%). ¹H NMR (400 MHz, CDCl₃): δ 8.14 (s, 1H), 8.01-7.92 (m, 2H), 7.36 (d, J=8.2 Hz, 2H), 5.02 (d, J=29.7 Hz, 1H), 4.45 (q, J=7.1 Hz, 2H), 4.36 (d, J=6.0 Hz, 2H), 1.46 (d, J=3.8 Hz, 9H), 1.43 (t, J=7.1 Hz, 3H).

### Methyl (2-(4-(((tert-butoxycarbonyl)amino)methyl)phenyl)thiazole-4-carbonyl)-L-serinate (35.4)

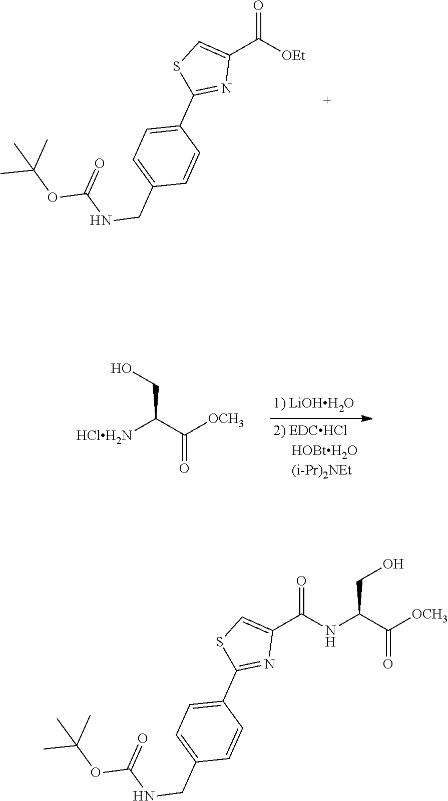

Ethyl 2-(4-(((tert-butoxycarbonyl)amino)methyl)phenyl)thiazole-4-carboxylate (5.003 g, 13.8 mmole) was dissolved in 4/1/1 THF/methanol/water (28 mL). Lithium hydroxide monohydrate (1.160 g, 27.6 mmole) was added and the mixture was stirred at RT for 3 hours. Water (50 mL) was added and the solution was treated with 1N aq. HCl to pH=4. The mixture was extracted with ethyl acetate (3×15 mL) and the combined organics were dried over magnesium sulfate, filtered, and concentrated. The residue was suspended in DCM (28 mL) and L-serine methyl ester hydrochloride (2.589 g, 16.6 mmole) was added. N,N-Diisopropylethyl amine (4.80 mL, 27.6 mmole), HOBt·H₂O (2.554 g, 16.7 mmole), and EDC·HCl (3.187 g, 16.6 mmole) were added and the mixture was stirred at RT for 18 hours. Water (50 mL) was added and the two layers were separated. The aqueous layer was extracted with DCM (2×20 mL) and the combined organics were dried over magnesium sulfate, filtered, and concentrated. The crude residue was purified by silica gel chromatography to provide methyl (2-(4-(((tert-butoxycarbonyl)amino)methyl)phenyl)thiazole-4-carbonyl)-L-serinate as a pale yellow solid (3.793 g, 63%).

¹H NMR (400 MHz, CDCl₃): δ 8.25 (d, J=7.5 Hz, 1H), 8.09 (s, 1H), 7.93-7.85 (m, 2H), 7.35 (d, J=7.9 Hz, 2H), 5.00 (s, 1H), 4.87 (dt, J=7.5, 3.8 Hz, 1H), 4.36 (d, J=6.0 Hz, 2H), 4.17-4.04 (m, 2H), 3.84 (s, 3H), 2.95 (t, J=6.1 Hz, 1H), 1.48 (s, 9H).

### Methyl O-(tert-butyldimethylsilyl)-N-(2-(4-((2-(2-methoxyethoxy)acetamido)methyl)phenyl)thiazole-4-carbonyl)-L-serinate (35.5)

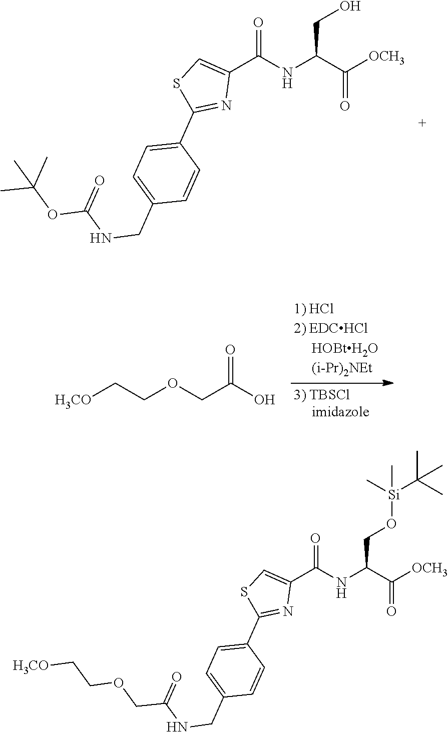 [SMILES: COC(=O)[C@H](CO)NC(=O)c1csc(-c2ccc(CNC(=O)OC(C)(C)C)cc2)n1 ; COCCOCC(=O)O ; COCCOCC(=O)NCc1ccc(-c2nc(C(=O)N[C@@H](CO[Si](C)(C)C(C)(C)C)C(=O)OC)cs2)cc1]

Methyl (2-(4-(((tert-butoxycarbonyl)amino)methyl)phenyl)thiazole-4-carbonyl)-L-serinate (0.700 g, 1.61 mmole) was dissolved in DCM (3.2 mL). HCl (4M solution in 1,4-dioxane, 1.60 mL, 6.40 mmole) was added and the mixture was stirred at RT for 2 hours, then was concentrated. The residue was suspended in DCM (3.2 mL) and 2-(2-methoxyethoxy)acetic acid (0.220 mL, 1.94 mmole) was added. N,N-Diisopropylethylamine (0.560 mL, 3.22 mmole), HOBt·H₂O (0.297 g, 1.94 mmole), and EDC·HCl (0.377 g, 1.97 mmole) were added and the mixture was stirred at RT for 3 days. Water (25 mL) was added and the mixture was extracted with DCM (3×10 mL). The combined organics were dried over magnesium sulfate, filtered, and concentrated. The residue was dissolved in DCM (3.2 mL) and imidazole (0.129 g, 1.89 mmole) and tert-butyldimethylchlorosilane (0.273, 1.81 mmole) were added. The mixture was stirred at RT for 18 hours and water (25 mL) was added. The mixture was extracted with DCM (3×10 mL). The combined organics were dried over magnesium sulfate, filtered, and concentrated. The crude residue was purified by silica gel chromatography to provide methyl O-(tert-butyldimethylsilyl)-N-(2-(4-((2-(2-methoxyethoxy)acetamido)methyl)phenyl)thiazole-4-carbonyl)-L-serinate as a colorless oil (0.838 g, 92%).

¹H NMR (400 MHz, CDCl₃): δ 8.22 (d, J=8.7 Hz, 1H), 8.10 (s, 1H), 7.98-7.89 (m, 2H), 7.48 (s, 1H), 7.43-7.35 (m, 2H), 4.86 (dt, J=8.7, 3.0 Hz, 1H), 4.54 (d, J=5.9 Hz, 2H), 4.21 (dd, J=10.1, 2.6 Hz, 1H), 4.08 (s, 2H), 3.95 (dd, J=10.1, 3.4 Hz, 1H), 3.79 (s, 3H), 3.74-3.66 (m, 2H), 3.56-3.49 (m, 2H), 3.28 (s, 3H), 0.92 (s, 9H), 0.08 (s, 3H), 0.06 (s, 3H).

### Methyl O-acetyl-N—(O-(tert-butyldimethylsilyl)-N-(2-(4-((2-(2-methoxyethoxy)acetamido)methyl)phenyl)thiazole-4-carbonyl)-L-seryl)-L-serinate (35.6)

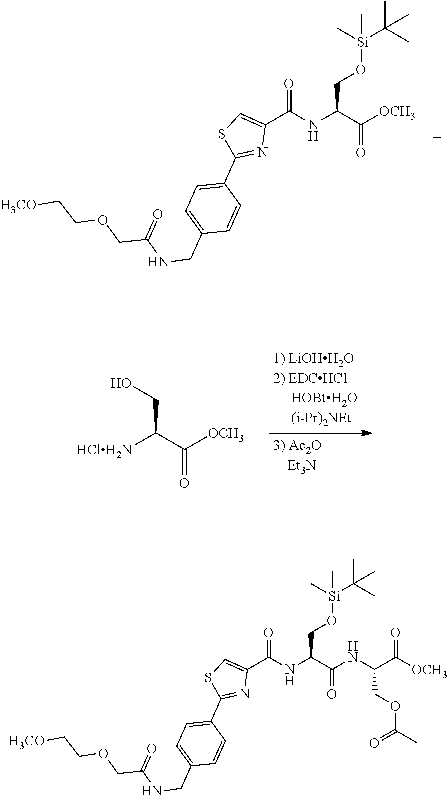

Methyl O-(tert-butyldimethylsilyl)-N-(2-(4-((2-(2-methoxyethoxy)acetamido)methyl)phenyl)thiazole-4-carbonyl)-L-serinate (0.838 g, 1.48 mmole) was dissolved in 4/1/1 THF/methanol/water (3 mL). Lithium hydroxide monohydrate (0.125 g, 2.98 mmole) was added and the mixture was stirred at RT for 2 hours. Water (25 mL) was added and the solution was treated with 1N aq. HCl to pH=4. The mixture was extracted with ethyl acetate (3×10 mL) and the combined organics were dried over magnesium sulfate, filtered, and concentrated. The residue was dissolved in DCM (3 mL) and L-serine methyl ester hydrochloride (0.282 g, 1.81 mmole) was added. N,N-Diisopropylethylamine (0.520 mL, 2.99 mmole), HOBt·H₂0 (0.277 g, 1.81 mmole), and EDC·HCl (0.343 g, 1.79 mmole) were added and the mixture was stirred at RT for 18 hours. Water (25 mL) was added and the mixture was extracted with DCM (3×10 mL). The combined organics were dried over magnesium sulfate, filtered, and concentrated. The residue was dissolved in DCM (3 mL) and triethylamine (0.230 mL, 1.64 mmole) and acetic anhydride (0.155 mL, 1.64 mmole) were added. The mixture was stirred at RT for 5 hours and water (25 mL) was added. The mixture was extracted with DCM (3×10 mL) and the combined organics were dried over magnesium sulfate, filtered, and concentrated. The crude residue was purified by silica gel chromatography to provide methyl O-acetyl-N—(O-(tert-butyldimethylsilyl)-N-(2-(4-((2-(2-methoxyethoxy)acetamido)methyl)phenyl)thiazole-4-carbonyl)-L-seryl)-L-serinate as a colorless oil (0.593 g, 58%).

¹H NMR (400 MHz, CDCl₃): δ 8.29-8.22 (m, 1H), 8.11 (d, J=2.4 Hz, 1H), 7.95 (dt, J=8.5, 2.0 Hz, 2H), 7.48 (t, J=6.4 Hz, 2H), 7.44-7.36 (m, 2H), 4.90 (tt, J=7.4, 3.8 Hz, 1H), 4.71-4.63 (m, 1H), 4.54 (d, J=6.0 Hz, 2H), 4.49 (ddd, J=11.0, 7.1, 3.9 Hz, 1H), 4.34 (dd, J=11.4, 3.6 Hz, 1H), 4.23 (dt, J=9.9, 3.4 Hz, 1H), 4.09 (d, J=4.9 Hz, 2H), 3.85-3.80 (m, 1H), 3.77 (d, J=4.2 Hz, 3H), 3.73-3.66 (m, 2H), 3.57-3.49 (m, 2H), 3.28 (s, 3H), 2.01 (s, 3H), 0.96 (s, 9H), 0.19-0.12 (m, 6H).

### Methyl 2-(2-(2-(4-((2-(2-methoxyethoxy)acetamido)methyl)phenyl)thiazole-4-carboxamido)acrylamido)acrylate (35)

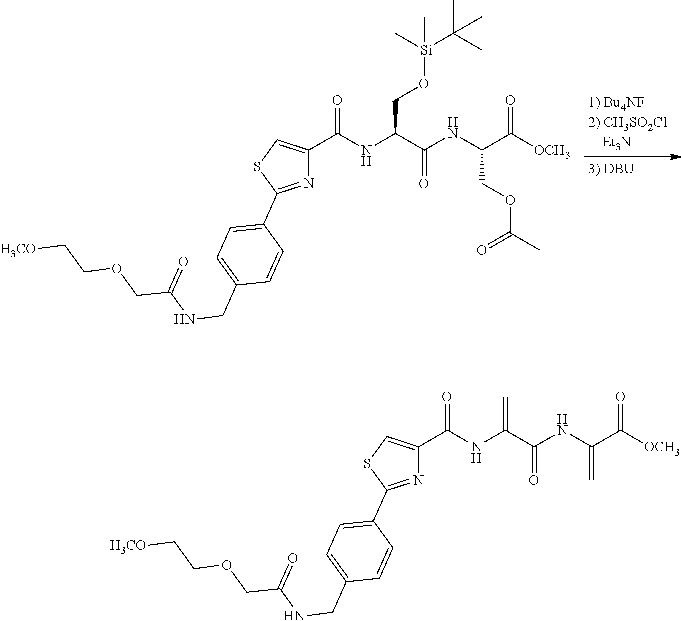 [SMILES: COCCOCC(=O)NCc1ccc(-c2nc(C(=O)N[C@@H](CO[Si](C)(C)C(C)(C)C)C(=O)N[C@@H](COC(C)=O)C(=O)OC)cs2)cc1 ; C=C(NC(=O)c1csc(-c2ccc(CNC(=O)COCCOC)cc2)n1)C(=O)NC(=C)C(=O)OC]

Methyl O-acetyl-N—(O-(tert-butyldimethylsilyl)-N-(2-(4-((2-(2-methoxyethoxy)acetamido)methyl)phenyl)thiazole-4-carbonyl)-L-seryl)-L-serinate (0.593 g, 0.853 mmole) was dissolved in THF (3.2 mL) and TBAF (1M solution in THF, 2.10 mL, 2.10 mmole) was added. The solution was stirred at RT for 90 minutes and water (25 mL) was added. The mixture was extracted with ethyl acetate (3×10 mL) and the combined organics were dried over magnesium sulfate, filtered, and concentrated. The residue was dissolved in DCM (3.2 mL) and cooled to 0° C. Triethylamine (0.180 mL, 1.28 mmole) and methanesulfonyl chloride (0.099 mL, 1.28 mmole) were added and the mixture was stirred at 0° for 60 minutes. Water (25 mL) was added and the mixture was extracted with DCM (3×10 mL). The combined organics were dried over magnesium sulfate, filtered, and concentrated. The residue was dissolved in THF (3.2 mL) and cooled to 0° C. DBU (0.180 mL, 1.20 mmole) was added and the mixture was stirred at 0° for 90 minutes. Water (25 mL) was added and the mixture was extracted with ethyl acetate (3×10 mL). The combined organics were dried over magnesium sulfate, filtered, and concentrated. The crude residue was purified by silica gel chromatography to provide methyl 2-(2-(2-(4-((2-(2-methoxyethoxy)acetamido)methyl)phenyl)thiazole-4-carboxamido)acrylamido)acrylate as a white solid (0.180 g, 42%).

¹H NMR (400 MHz, CDCl₃): δ 10.02 (s, 1H), 8.55 (s, 1H), 8.15 (s, 1H), 8.04-7.94 (m, 2H), 7.48 (s, 1H), 7.44-7.38 (m, 2H), 6.79 (d, J=2.2 Hz, 1H), 6.71 (s, 1H), 6.04 (d, J=1.3 Hz, 1H), 5.51 (t, J=1.9 Hz, 1H), 4.54 (d, J=6.0 Hz, 2H), 4.08 (s, 2H), 3.91 (s, 3H), 3.74-3.67 (m, 2H), 3.59-3.50 (m, 2H), 3.28 (s, 3H).

### Compound 36: Methyl 2-(2-(2-(4-((tetrahydro-2H-pyran-4-carboxamido)methyl)phenyl)thiazole-4-carboxamido)acrylamido)acrylate

### Methyl O-(tert-butyldimethylsilyl)-N-(2-(4-((tetrahydro-2H-pyran-4-carboxamido)methyl)phenyl)thiazole-4-carbonyl)-L-serinate (36.1)

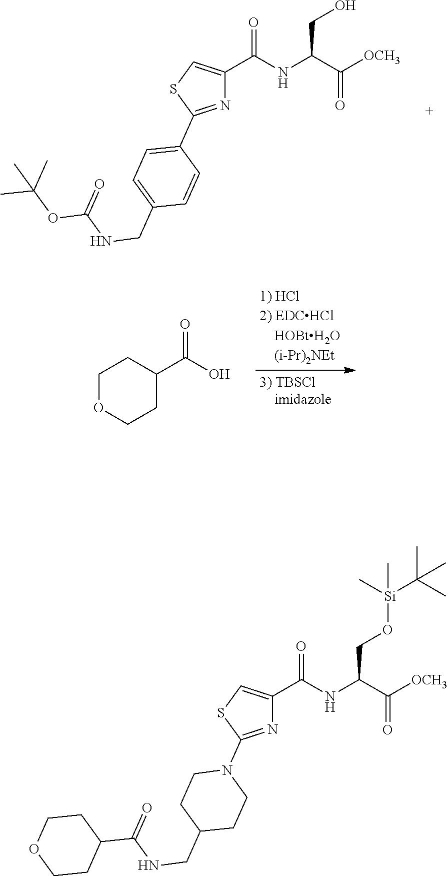 [SMILES: COC(=O)[C@H](CO)NC(=O)c1csc(-c2ccc(CNC(=O)OC(C)(C)C)cc2)n1 ; O=C(O)C1CCOCC1 ; COC(=O)[C@H](CO[Si](C)(C)C(C)(C)C)NC(=O)c1csc(N2CCC(CNC(=O)C3CCOCC3)CC2)n1]

Methyl (2-(4-(((tert-butoxycarbonyl)amino)methyl)phenyl)thiazole-4-carbonyl)-L-serinate (Example 35.4, 0.603 g, 1.38 mmole) was dissolved in DCM (2.8 mL). HCl (4M solution in 1,4-dioxane) was added and the mixture was stirred at RT for 2 hours, then was concentrated. The residue was suspended in DCM (2.8 mL) and tetrahydro-2H-pyran-4-carboxylic acid (0.218 g, 1.40 mmole) was added. N,N-Diisopropylethyl amine (0.480 mL, 2.76 mmole), HOBt·H₂O (0.256 g, 1.67 mmole), and EDC·HCl (0.324 g, 1.69 mmole) were added and the mixture was stirred at RT for 18 hours. Water (25 mL) was added and the mixture was extracted with DCM (3×10 mL). The combined organics were dried over magnesium sulfate, filtered, and concentrated. The residue was dissolved in DCM (5.6 mL) and imidazole (0.112 g, 1.65 mmole) and tert-butyldimethylchlorosilane (0.237 g, 1.57 mmole) were added. The mixture was stirred at RT for 5 hours and water (25 mL) was added. The mixture was extracted with DCM (3×10 mL) and the combined organics were dried over magnesium sulfate, filtered, and concentrated. The crude residue was purified by silica gel chromatography to provide methyl O-(tert-butyldimethylsilyl)-N-(2-(4-((tetrahydro-2H-pyran-4-carboxamido)methyl)phenyl)thiazole-4-carbonyl)-L-serinate as a pale yellow solid (0.535 g, 69%).

¹H NMR (400 MHz, CDCl₃): δ 8.22 (d, J=8.6 Hz, 1H), 8.08 (s, 1H), 7.96-7.88 (m, 2H), 7.38-7.31 (m, 2H), 5.94 (s, 1H), 4.89-4.80 (m, 1H), 4.51 (d, J=5.8 Hz, 2H), 4.21 (dd, J=10.1, 2.6 Hz, 1H), 4.03 (ddd, J=11.5, 4.0, 2.2 Hz, 2H), 3.95 (dd, J=10.1, 3.4 Hz, 1H), 3.79 (s, 3H), 3.43 (td, J=11.4, 2.9 Hz, 2H), 2.42 (tt, J=11.1, 4.5 Hz, 1H), 1.94-1.74 (m, 4H), 0.92 (s, 9H), 0.07 (s, 3H), 0.06 (s, 3H).

### Methyl O-acetyl-N—(O-(tert-butyldimethylsilyl)-N-(2-(4-((tetrahydro-2H-pyran-4-carboxamido)methyl)phenyl)thiazole-4-carbonyl)-L-seryl)-L-serinate (36.2)

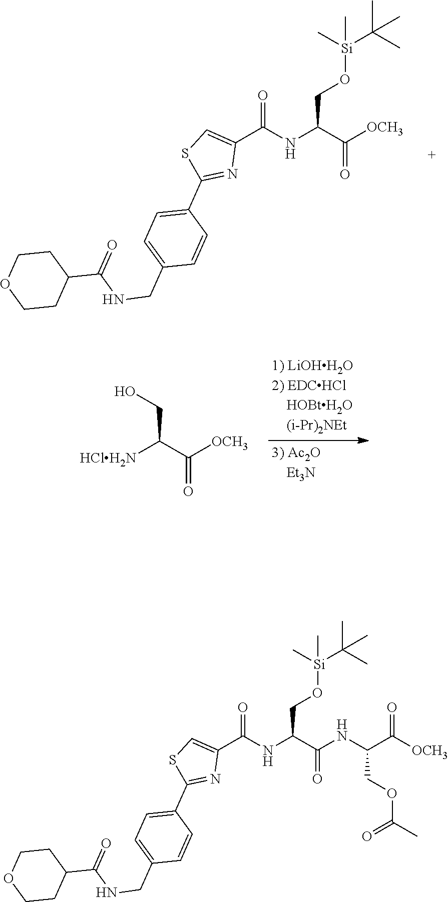

Methyl O-(tert-butyldimethylsilyl)-N-(2-(4-((tetrahydro-2H-pyran-4-carboxamido)methyl)phenyl)thiazole-4-carbonyl)-L-serinate (0.535 g, 0.952 mmole) was dissolved in 4/1/1 THF/methanol/water (2 mL). Lithium hydroxide monohydrate (0.063 g, 1.60 mmole) was added and the mixture was stirred at RT for 3 hours. Water (25 mL) was added and the solution was treated with 1N aq. HCl to pH=4. The mixture was extracted with ethyl acetate (3×10 mL) and the combined organics were dried over magnesium sulfate, filtered, and concentrated. The residue was dissolved in DCM (4 mL) and L-serine methyl ester hydrochloride (0.182 g, 1.17 mmole) was added. N,N-Diisopropylethylamine (0.340 mL, 1.95 mmole), HOBt·H₂O (0.175 g, 1.14 mmole), and EDC·HCl (0.223 g, 1.16 mmole) were added and the mixture was stirred at RT for 3 days. Water (25 mL) was added and the mixture was extracted with DCM (3×10 mL). The combined organics were dried over magnesium sulfate, filtered, and concentrated. The residue was dissolved in DCM (4 mL) and triethylamine (0.150 mL, 1.07 mmole) and acetic anhydride (0.099 mL, 1.09 mmole) were added. The mixture was stirred at RT for 3 hours and water (25 mL) was added. The mixture was extracted with DCM (3×10 mL) and the combined organics were dried over magnesium sulfate, filtered, and concentrated. The crude residue was purified by silica gel chromatography to provide methyl O-acetyl-N—(O-(tert-butyldimethylsilyl)-N-(2-(4-((tetrahydro-2H-pyran-4-carboxamido)methyl)phenyl)thiazole-4-carbonyl)-L-seryl)-L-serinate as a colorless oil (0.441 g, 88%).

¹H NMR (400 MHz, CDCl₃): δ 8.26 (d, J=7.0 Hz, 1H), 8.10 (d, J=2.4 Hz, 1H), 7.97-7.90 (m, 2H), 7.48 (d, J=7.8 Hz, 1H), 7.38-7.30 (m, 2H), 5.90 (t, J=5.9 Hz, 1H), 4.90 (dq, J=7.0, 3.5 Hz, 1H), 4.66 (td, J=7.3, 3.7 Hz, 1H), 4.51 (d, J=5.8 Hz, 2H), 4.47 (dd, J=6.7, 4.0 Hz, 1H), 4.34 (dd, J=11.4, 3.6 Hz, 1H), 4.23 (dd, J=9.8, 3.7 Hz, 1H), 4.04 (ddd, J=11.5, 4.2, 2.3 Hz, 2H), 3.84-3.79 (m, 1H), 3.77 (s, 3H), 3.43 (td, J=11.5, 2.9 Hz, 2H), 2.42 (tt, J=11.0, 4.4 Hz, 1H), 2.01 (s, 3H), 1.92-1.75 (m, 4H), 0.96 (s, 9H), 0.17 (s, 3H), 0.16 (s, 3H).

### Methyl 2-(2-(2-(4-((tetrahydro-2H-pyran-4-carboxamido)methyl)phenyl)thiazole-4-carboxamido)acrylamido)acrylate (36)

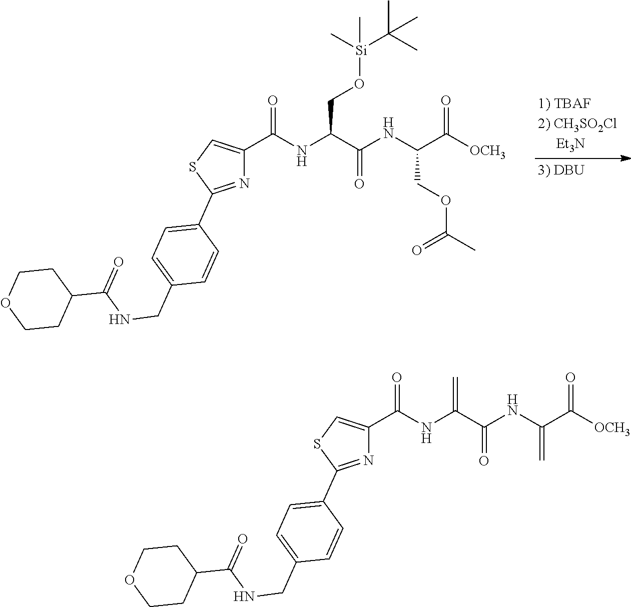 [SMILES: COC(=O)[C@H](COC(C)=O)NC(=O)[C@H](CO[Si](C)(C)C(C)(C)C)NC(=O)c1csc(-c2ccc(CNC(=O)C3CCOCC3)cc2)n1 ; C=C(NC(=O)c1csc(-c2ccc(CNC(=O)C3CCOCC3)cc2)n1)C(=O)NC(=C)C(=O)OC]

Methyl O-acetyl-N—(O-(tert-butyldimethylsilyl)-N-(2-(4-((tetrahydro-2H-pyran-4-carboxamido)methyl)phenyl)thiazole-4-carbonyl)-L-seryl)-L-serinate (0.441 g, 0.638 mmole) was dissolved in THF (1.3 mL) and TBAF (1M solution in THF, 1.60 mL, 1.60 mmole) was added. The mixture was stirred at RT for 2 hours and water (25 mL) was added. The mixture was extracted with ethyl acetate (3×10 mL) and the combined organics were dried over magnesium sulfate, filtered, and concentrated. The residue was dissolved in DCM (2.6 mL) and cooled to 0° C. Triethylamine (0.135 mL, 0.963 mmole) and methanesulfonyl chloride (0.075 mL, 0.969 mmole) were added and the mixture was stirred at 0° for 60 minutes. Water (25 mL) was added and the mixture was extracted with DCM (3×10 mL). The combined organics were dried over magnesium sulfate, filtered, and concentrated. The residue was dissolved in THF (2.6 mL) and cooled to 0° C. DBU (0.150 mL, 1.00 mmole) was added and the mixture was stirred at 0° for 45 minutes. Water (25 mL) was added and the mixture was extracted with ethyl acetate (3×10 mL). The combined organics were filtered, then dried over magnesium sulfate, filtered, and concentrated. The residue was suspended in a small amount of ethyl acetate and the solid was collected by filtration. The two crops were combined to provide methyl 2-(2-(2-(4-((tetrahydro-2H-pyran-4-carboxamido)methyl)phenyl)thiazole-4-carboxamido)acrylamido)acrylate as a white solid (0.101 g, 32%).

¹H NMR (400 MHz, CDCl₃): δ 10.01 (s, 1H), 8.56 (s, 1H), 8.14 (s, 1H), 8.02-7.92 (m, 2H), 7.36 (d, J=8.0 Hz, 2H), 6.78 (d, J=2.2 Hz, 1H), 6.70 (s, 1H), 6.04 (d, J=1.3 Hz, 1H), 5.85 (s, 1H), 5.51 (t, J=1.9 Hz, 1H), 4.52 (d, J=5.8 Hz, 2H), 4.04 (ddd, J=11.6, 4.3, 2.4 Hz, 2H), 3.91 (s, 3H), 3.43 (td, J=11.4, 3.0 Hz, 2H), 2.41 (tt, J=11.0, 4.6 Hz, 1H), 1.93-1.74 (m, 4H).

### Compound 37: Methyl 2-(2-(2-(4-((6-chlorohexanamido)methyl)phenyl)thiazole-4-carboxamido)acrylamido)acrylate

### Methyl O-(tert-butyldimethylsilyl)-N-(2-(4-((6-chlorohexanamido)methyl)phenyl)thiazole-4-carbonyl)-L-serinate (37.1)

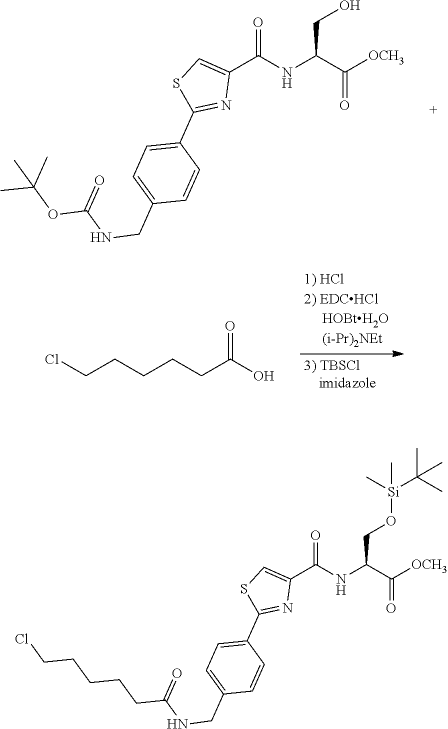 [SMILES: COC(=O)[C@H](CO)NC(=O)c1csc(-c2ccc(CNC(=O)OC(C)(C)C)cc2)n1 ; O=C(O)CCCCCCl ; COC(=O)[C@H](CO[Si](C)(C)C(C)(C)C)NC(=O)c1csc(-c2ccc(CNC(=O)CCCCCCl)cc2)n1]

Methyl (2-(4-(((tert-butoxycarbonyl)amino)methyl)phenyl)thiazole-4-carbonyl)-L-serinate (Example 35.4, 0.603 g, 1.38 mmole) was dissolved in DCM (2.8 mL). HCl (4M solution in 1,4-dioxane, 1.40 mL, 5.60 mmole) was added and the mixture was stirred at RT for 3 hours, then was concentrated. The residue was suspended in DCM (2.8 mL) and a solution of 6-chlorohexanoic acid (0.249 g, 1.65 mmole) in DCM (0.5 mL) was added. N,N-Diisopropylethylamine (0.480 mL, 2.76 mmole), HOBt·H₂O (0.254 g, 1.66 mmole), and EDC·HCl (0.320 g, 1.67 mmole) were added and the mixture was stirred at RT for 3 days. Water (25 mL) was added and the mixture was extracted with DCM (3×10 mL). The combined organics were dried over magnesium sulfate, filtered, and concentrated. The residue was dissolved in DCM (2.8 mL) and imidazole (0.114 g, 1.67 mmole) and tert-butyldimethylchlorosilane (0.231 g, 1.53 mmole) were added. The mixture was stirred at RT for 3 hours and water (25 mL) was added. The mixture was extracted with DCM (3×10 mL) and the combined organics were dried over magnesium sulfate, filtered, and concentrated. The crude residue was purified by silica gel chromatography to provide methyl O-(tert-butyldimethylsilyl)-N-(2-(4-((6-chlorohexanamido)methyl)phenyl)thiazole-4-carbonyl)-L-serinate as a white solid (0.612 g, 76%).

¹H NMR (400 MHz, CDCl₃): δ 8.21 (d, J=8.6 Hz, 1H), 8.07 (s, 1H), 7.95-7.89 (m, 2H), 7.39-7.31 (m, 2H), 5.93 (t, J=6.0 Hz, 1H), 4.89-4.81 (m, 1H), 4.50 (d, J=5.8 Hz, 2H), 4.21 (dd, J=10.1, 2.7 Hz, 1H), 3.95 (dd, J=10.1, 3.4 Hz, 1H), 3.79 (s, 3H), 3.54 (t, J=6.6 Hz, 2H), 2.28 (t, J=7.5 Hz, 2H), 1.80 (dt, J=8.1, 6.7 Hz, 2H), 1.73 (tt, J=8.3, 6.4 Hz, 2H), 1.56-1.47 (m, 2H), 0.92 (s, 9H), 0.08 (s, 3H), 0.06 (s, 3H).

### Methyl O-acetyl-N—(O-(tert-butyldimethylsilyl)-N-(2-(4-((6-chlorohexanamido)methyl)phenyl)thiazole-4-carbonyl)-L-seryl)-L-serinate (37.2)

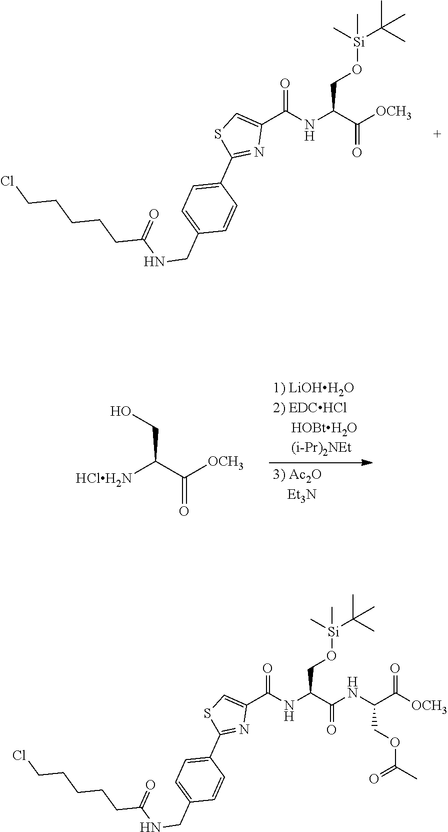

Methyl O-(tert-butyldimethylsilyl)-N-(2-(4-((6-chlorohexanamido)methyl)phenyl)thiazole-4-carbonyl)-L-serinate (0.612 g, 1.05 mmole) was dissolved in 4/1/1 THF/methanol/water (2 mL). Lithium hydroxide monohydrate (0.065 g, 1.55 mmole) was added and the mixture was stirred at RT for 3 hours. Water (25 mL) was added and the solution was treated with 1N aq. HCl to pH=4. The mixture was extracted with ethyl acetate (3×10 mL) and the combined organics were dried over magnesium sulfate, filtered, and concentrated. The residue was dissolved in DCM (2 mL) and L-serine methyl ester hydrochloride (0.192 g, 1.23 mmole) was added. N,N-Diisopropylethylamine (0.370 mL, 2.12. mmole), HOBt·H₂O (0.199 g, 1.30 mmole), and EDC·HCl (0.240 g, 1.25 mmole) were added and the mixture was stirred at RT for 18 hours. Water (25 mL) was added and the mixture was extracted with DCM (3×10 mL). The combined organics were dried over magnesium sulfate, filtered, and concentrated. The residue was dissolved in DCM (4 mL) and triethylamine (0.165 mL, 1.18 mmole) and acetic anhydride (0.110 mL, 1.16 mmole) were added. The mixture was stirred at RT for 3 hours and water (25 mL) was added. The mixture was extracted with DCM (3×10 mL) and the combined organics were dried over magnesium sulfate, filtered, and concentrated. The crude residue was purified by silica gel chromatography to provide methyl O-acetyl-N—(O-(tert-butyldimethylsilyl)-N-(2-(4-((6-chlorohexanamido)methyl)phenyl)thiazole-4-carbonyl)-L-seryl)-L-serinate as a colorless oil (0.288 g, 39%).

¹H NMR (400 MHz, CDCl₃): δ 8.26 (d, J=7.2 Hz, 1H), 8.10 (d, J=2.3 Hz, 1H), 7.98-7.88 (m, 2H), 7.47 (d, J=7.8 Hz, 1H), 7.39-7.31 (m, 2H), 5.84 (t, J=5.6 Hz, 1H), 4.90 (dt, J=7.5, 3.7 Hz, 1H), 4.66 (td, J=7.3, 3.7 Hz, 1H), 4.54-4.47 (m, 3H), 4.34 (dd, J=11.4, 3.6 Hz, 1H), 4.22 (dt, J=9.8, 3.4 Hz, 1H), 3.84-3.79 (m, 1H), 3.77 (d, J=4.1 Hz, 3H), 3.55 (t, J=6.6 Hz, 2H), 2.27 (t, J=7.5 Hz, 2H), 2.05 (s, 3H), 1.86-1.77 (m, 2H), 1.77-1.66 (m, 2H), 1.56-1.48 (m, 2H), 0.96 (s, 9H), 0.16 (t, J=4.8 Hz, 6H).

### Methyl 2-(2-(2-(4-((6-chlorohexanamido)methyl)phenyl)thiazole-4-carboxamido)acrylamido)acrylate (37)

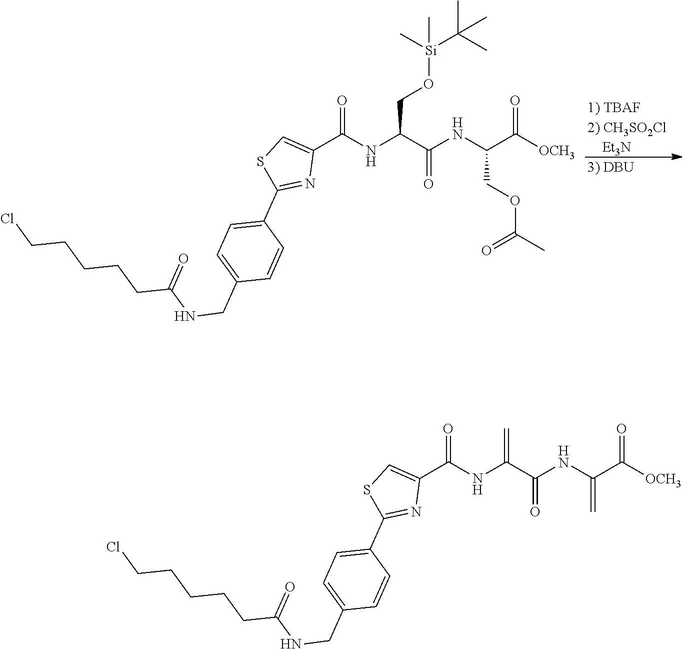 [SMILES: COC(=O)[C@H](COC(C)=O)NC(=O)[C@H](CO[Si](C)(C)C(C)(C)C)NC(=O)c1csc(-c2ccc(CNC(=O)CCCCCCl)cc2)n1 ; C=C(NC(=O)c1csc(-c2ccc(CNC(=O)CCCCCCl)cc2)n1)C(=O)NC(=C)C(=O)OC]

Methyl O-acetyl-N—(O-(tert-butyldimethylsilyl)-N-(2-(4-((6-chlorohexanamido)methyl)phenyl)thiazole-4-carbonyl)-L-seryl)-L-serinate (0.288 g, 0.405 mmole) was dissolved in THF (1.6 mL). TBAF (1M solution in THF, 1.00 mL, 1.00 mmole) was added and the solution was stirred at RT for 2 hours. Water (25 mL) was added and the mixture was extracted with ethyl acetate (3×10 mL). The combined organics were dried over magnesium sulfate, filtered, and concentrated. The residue was dissolved in DCM (1.6 mL) and cooled to 0° C. Triethylamine (0.086 mL, 0.614 mmole) and methanesulfonyl chloride (0.048 mL, 0.620 mmole) were added and the mixture was stirred at 0° for 45 minutes. Water (25 mL) was added and the mixture was extracted with DCM (3×10 mL). The combined organics were dried over magnesium sulfate, filtered, and concentrated. The residue was dissolved in THF (1.6 mL) and cooled to 0° C. DBU (0.091 mL, 0.608 mmole) was added and the mixture was stirred at 0° for 30 minutes. Water (25 mL) was added and the mixture was extracted with ethyl acetate (3×10 mL). The combined organics were dried over magnesium sulfate, filtered, and concentrated. The crude residue was purified by silica gel chromatography to provide methyl 2-(2-(2-(4-((6-chlorohexanamido)methyl)phenyl)thiazole-4-carboxamido)acrylamido)acrylate as a white solid (0.100 g, 48%).

¹H NMR (400 MHz, CDCl₃): δ 10.03 (s, 1H), 8.58 (s, 1H), 8.15 (s, 1H), 8.04-7.94 (m, 2H), 7.44-7.34 (m, 2H), 6.80 (d, J=2.3 Hz, 1H), 6.73 (s, 1H), 6.06 (d, J=1.3 Hz, 1H), 5.86 (s, 1H), 5.53 (t, J=1.9 Hz, 1H), 4.53 (d, J=5.8 Hz, 2H), 3.93 (s, 3H), 3.57 (t, J=6.6 Hz, 2H), 2.30 (t, J=7.5 Hz, 2H), 1.90-1.79 (m, 2H), 1.75 (tt, J=8.3, 6.4 Hz, 2H), 1.58-1.49 (m, 2H).

### Compound 38. Methyl 2-(2-(2-(4-((3-((tert-butoxycarbonyl)amino)propanamido)methyl) phenyl)thiazole-4-carboxamido)acrylamido)acrylate

### Methyl N-(2-(4-((3-((tert-butoxycarbonyl)amino)propanamido)methyl)phenyl)thiazole-4-carbonyl)-O-(tert-butyldimethylsilyl)-L-serinate (38.1)

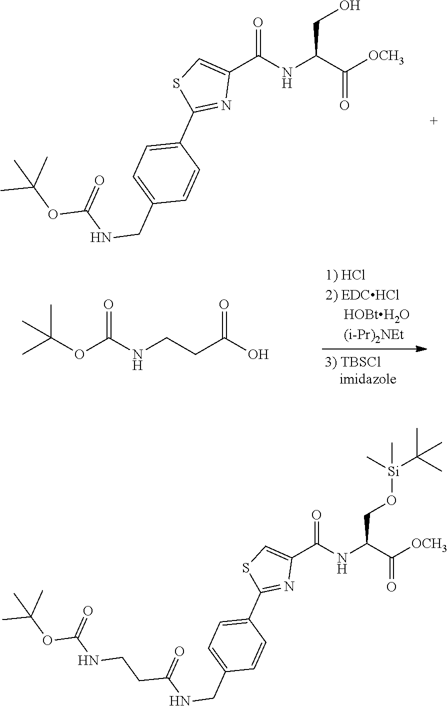 [SMILES: COC(=O)[C@H](CO)NC(=O)c1csc(-c2ccc(CNC(=O)OC(C)(C)C)cc2)n1 ; CC(C)(C)OC(=O)NCCC(=O)O ; COC(=O)[C@H](CO[Si](C)(C)C(C)(C)C)NC(=O)c1csc(-c2ccc(CNC(=O)CCNC(=O)OC(C)(C)C)cc2)n1]

Methyl (2-(4-(((tert-butoxycarbonyl)amino)methyl)phenyl)thiazole-4-carbonyl)-L-serinate (Example 35.4, 0.612 g, 1.41 mmole) was dissolved in DCM (5.6 mL). HCl (4M solution in 1,4-dioxane, 1.40 mL, 5.60 mmole) was added and the mixture was stirred at RT for 3 hours, then was concentrated. The residue was suspended in DCM (5.6 mL) and (tert-butoxycarbonyl)-□-alanine (0.317 g, 1.68 mmole) was added. N,N-Diisopropylethylamine (0.480 mL, 2.76 mmole), HOBt·H₂O (0.260 g, 1.70 mmole), and EDC·HCl (0.320 g, 1.67 mmole) were added and the mixture was stirred at RT for 3 days. Water (25 mL) was added and the mixture was extracted with DCM (3×10 mL). The combined organics were dried over magnesium sulfate, filtered, and concentrated. The residue was dissolved in DCM (5.6 mL) and imidazole (0.112 g, 1.65 mmole) and tert-butyldimethylchlorosilane (0.231 g, 1.53 mmole) were added. The mixture was stirred at RT for 3 hours and water (25 mL) was added. The mixture was extracted with DCM (3×10 mL) and the combined organics were dried over magnesium sulfate, filtered, and concentrated. The crude residue was purified by silica gel chromatography to provide methyl N-(2-(4-((3-((tert-butoxycarbonyl)amino)propanamido)methyl)phenyl)thiazole-4-carbonyl)-O-(tert-butyldimethylsilyl)-L-serinate as a colorless solid (0.626 g, 72%).

¹H NMR (400 MHz, CDCl₃): δ 8.21 (d, J=8.7 Hz, 1H), 8.09 (s, 1H), 7.97-7.88 (m, 2H), 7.40-7.32 (m, 2H), 6.15 (s, 1H), 5.15 (s, 1H), 4.86 (dt, J=8.7, 3.1 Hz, 1H), 4.50 (d, J=5.8 Hz, 2H), 4.21 (dd, J=10.0, 2.7 Hz, 1H), 3.95 (dd, J=10.1, 3.4 Hz, 1H), 3.79 (s, 3H), 3.45 (q, J=6.2 Hz, 2H), 2.49 (t, J=6.0 Hz, 2H), 1.42 (s, 9H), 0.92 (s, 9H), 0.07 (s, 3H), 0.06 (s, 3H).

### Methyl O-acetyl-N—(N-(2-(4-((3-((tert-butoxycarbonyl)amino)propanamido)methyl)phenyl) thiazole-4-carbonyl)-O-(tert-butyldimethylsilyl)-L-seryl)-L-serinate (38.2)

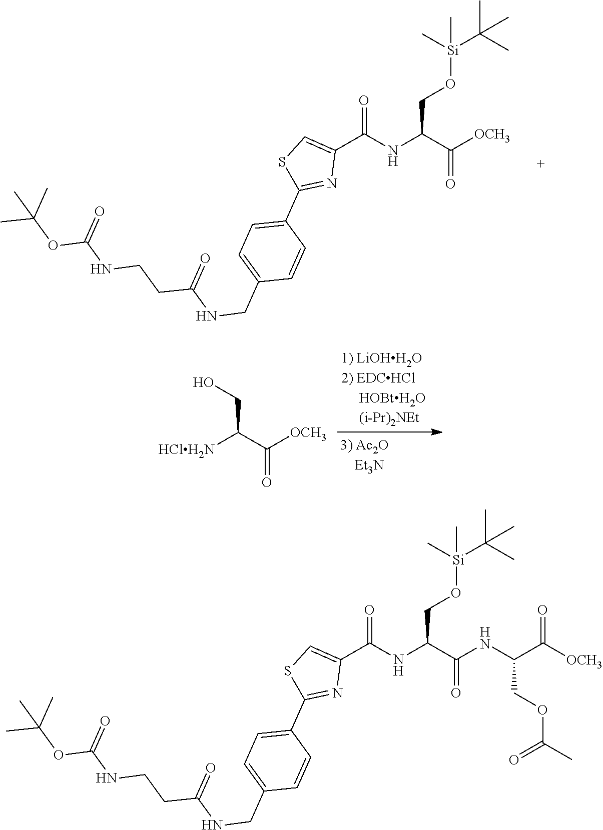

Methyl N-(2-(4-((3-((tert-butoxycarbonyl)amino)propanamido)methyl)phenyl)thiazole-4-carbonyl)-O-(tert-butyldimethylsilyl)-L-serinate (0.626 g, 1.01 mmole) was dissolved in 4/1/1 THF/methanol/water (2 mL). Lithium hydroxide monohydrate (0.066 g, 1.57 mmole) was added and the mixture was stirred at RT for 2 hours. Water (25 mL) was added and the solution was treated with 1N aq. HCl to pH=4. The mixture was extracted with ethyl acetate (3×10 mL) and the combined organics were dried over magnesium sulfate, filtered, and concentrated. The residue was dissolved in DCM (4 mL). L-Serine methyl ester hydrochloride (0.188 g, 1.21 mmole) was added followed by N,N-diisopropylethylamine (0.350 mL, 2.01 mmole), HOBt·H₂O (0.192 g, 1.25 mmole), and EDC·HCl (0.232 g, 1.21 mmole). The mixture was stirred at RT for 18 hours and water (25 mL) was added. The mixture was extracted with DCM (3×10 mL) and the combined organics were dried over magnesium sulfate, filtered, and concentrated. The residue was dissolved in DCM (4 mL) and triethylamine (0.160 mL, 1.14 mmole) and acetic anhydride (0.105 mL, 1.11 mmole) were added. The mixture was stirred at RT for 18 hours and water (25 mL) was added. The mixture was extracted with DCM (3×10 mL) and the combined organics were dried over magnesium sulfate, filtered, and concentrated. The crude residue was purified by silica gel chromatography to provide methyl O-acetyl-N—(N-(2-(4-((3-((tert-butoxycarbonyl)amino)propanamido)methyl)phenyl)thiazole-4-carbonyl)-O-(tert-butyldimethylsilyl)-L-seryl)-L-serinate as a colorless oil (0.315 g, 42%).

¹H NMR (400 MHz, CDCl₃): δ 8.25 (dd, J=7.2, 3.4 Hz, 1H), 8.09 (d, J=4.6 Hz, 1H), 7.97-7.89 (m, 2H), 7.48 (d, J=7.8 Hz, 1H), 7.39-7.31 (m, 2H), 6.26 (s, 1H), 5.18 (s, 1H), 4.89 (tt, J=6.6, 3.8 Hz, 1H), 4.66 (td, J=7.3, 3.8 Hz, 1H), 4.53-4.45 (m, 3H), 4.34 (dd, J=11.4, 3.6 Hz, 1H), 4.22 (dt, J=9.9, 3.8 Hz, 1H), 3.84-3.78 (m, 1H), 3.77 (s, 3H), 3.45 (q, J=6.2 Hz, 2H), 2.49 (t, J=6.0 Hz, 2H), 2.01 (s, 3H), 1.42 (s, 9H), 0.96 (s, 9H), 0.16 (s, 3H), 0.15 (s, 3H).

### Methyl 2-(2-(2-(4-((3-((tert-butoxycarbonyl)amino)propanamido)methyl)phenyl)thiazole-4-carboxamido)acrylamido)acrylate (38)

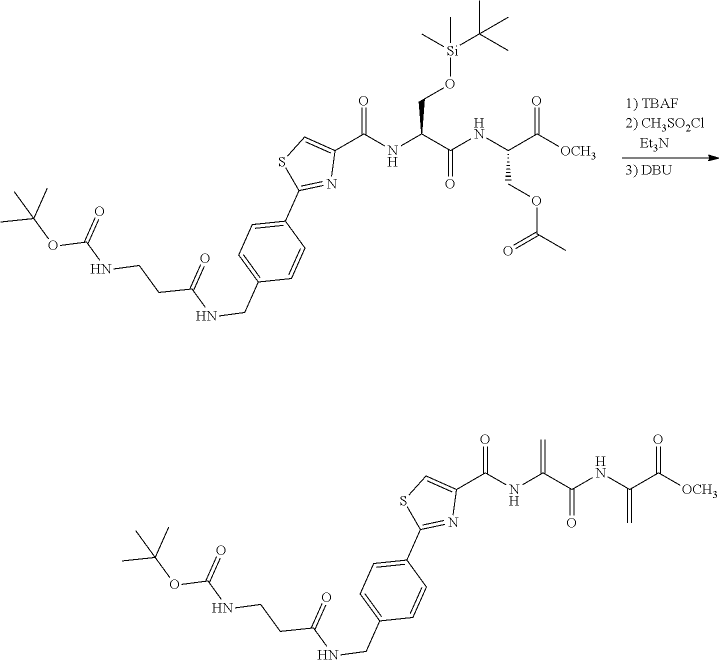 [SMILES: COC(=O)[C@H](COC(C)=O)NC(=O)[C@H](CO[Si](C)(C)C(C)(C)C)NC(=O)c1csc(-c2ccc(CNC(=O)CCNC(=O)OC(C)(C)C)cc2)n1 ; C=C(NC(=O)c1csc(-c2ccc(CNC(=O)CCNC(=O)OC(C)(C)C)cc2)n1)C(=O)NC(=C)C(=O)OC]

Methyl O-acetyl-N—(N-(2-(4-((3-((tert-butoxycarbonyl)amino)propanamido)methyl)phenyl)thiazole-4-carbonyl)-O-(tert-butyldimethylsilyl)-L-seryl)-L-serinate (0.315 g, 0.420 mmole) was dissolved in THF (1.6 mL). TBAF (1M solution in THF, 1.05 mL, 1.05 mmole) was added and the solution was stirred at RT for 2 hours. Water (25 mL) was added and the mixture was extracted with ethyl acetate (3×10 mL). The combined organics were dried over magnesium sulfate, filtered, and concentrated. The residue was dissolved in DCM (1.6 mL) and cooled to 0° C. Triethylamine (0.089 mL, 0.635 mmole) and methanesulfonyl chloride (0.049 mL, 0.633 mmole) were added and the mixture was stirred at 0° for 2 hours, then at RT for 18 hours. Water (25 mL) was added and the mixture was extracted with DCM (3×10 mL). The combined organics were dried over magnesium sulfate, filtered, and concentrated. The residue was dissolved in THF (1.6 mL) and cooled to 0° C. DBU (0.100 mL, 0.669 mmole) was added and the mixture was stirred at 0° for 30 minutes. Water (25 mL) was added and the mixture was extracted with ethyl acetate (3×10 mL). The combined organics were dried over magnesium sulfate, filtered, and concentrated. The crude residue was purified by silica gel chromatography to provide methyl 2-(2-(2-(4-((3-((tert-butoxycarbonyl)amino)propanamido)methyl)phenyl)thiazole-4-carboxamido)acrylamido)acrylate as a white solid (0.096 g, 41%).

¹H NMR (400 MHz, CDCl₃): δ 10.01 (s, 1H), 8.55 (s, 1H), 8.14 (s, 1H), 8.01-7.93 (m, 2H), 7.41-7.33 (m, 2H), 6.78 (d, J=2.2 Hz, 1H), 6.71 (s, 1H), 6.12 (s, 1H), 6.03 (d, J=1.3 Hz, 1H), 5.50 (t, J=1.9 Hz, 1H), 5.15 (s, 1H), 4.50 (d, J=5.9 Hz, 2H), 3.90 (s, 3H), 3.46 (q, J=6.2 Hz, 2H), 2.49 (t, J=6.0 Hz, 2H), 1.43 (s, 9H).

### Compound 39: Methyl 2-(2-(2-(4-((6-azidohexanamido)methyl)phenyl)thiazole-4-carboxamido)acrylamido)acrylate

### Methyl N-(2-(4-((6-bromohexanamido)methyl)phenyl)thiazole-4-carbonyl)-O-(tert-butyldimethylsilyl)-L-serinate (39.1)

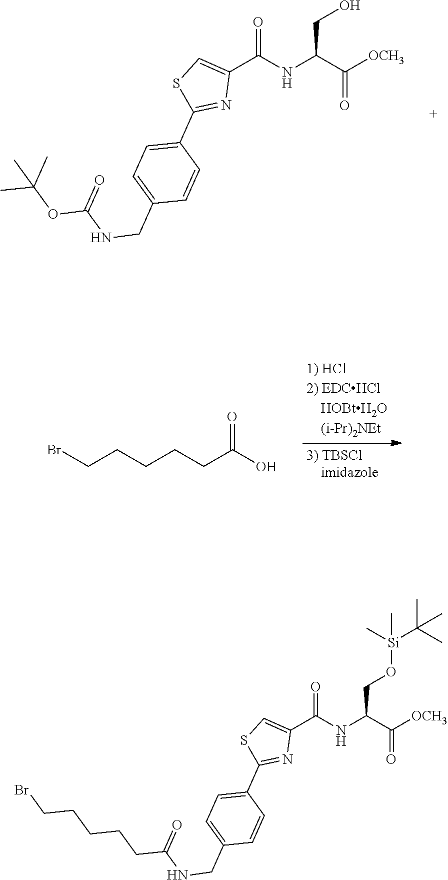 [SMILES: COC(=O)[C@H](CO)NC(=O)c1csc(-c2ccc(CNC(=O)OC(C)(C)C)cc2)n1 ; O=C(O)CCCCCBr ; COC(=O)[C@H](CO[Si](C)(C)C(C)(C)C)NC(=O)c1csc(-c2ccc(CNC(=O)CCCCCBr)cc2)n1]

Methyl (2-(4-(((tert-butoxycarbonyl)amino)methyl)phenyl)thiazole-4-carbonyl)-L-serinate (Example 35.4, 1.202 g, 2.76 mmole) was dissolved in DCM (20 mL). HCl (4M solution in 1,4-dioxane, 2.80 mL, 11.2 mmole) was added and the mixture was stirred at RT for 5 hours, then was concentrated. The residue was suspended in DCM (10 mL) and 6-bromohexanoic acid (0.673 g, 3.45 mmole) was added. N,N-Diisopropylethylamine (0.960 mL, 5.51 mmole), HOBt·H₂O (0.572 g, 3.74 mmole), and EDC·HCl (0.638 g, 3.33 mmole) were added and the mixture was stirred at RT for 18 hours. Water (25 mL) was added and the mixture was extracted with DCM (3×10 mL). The combined organics were dried over magnesium sulfate, filtered, and concentrated. The residue was dissolved in DCM (10 mL) and imidazole (0.214 g, 3.14 mmole) and tert-butyldimethylchlorosilane (0.474 g, 3.14 mmole) were added. The mixture was stirred at RT for 3 hours and water (25 mL) was added. The mixture was extracted with DCM (3×10 mL) and the combined organics were dried over magnesium sulfate, filtered, and concentrated. The crude residue was purified by silica gel chromatography to provide methyl N-(2-(4-((6-bromohexanamido)methyl)phenyl)thiazole-4-carbonyl)-O-(tert-butyldimethylsilyl)-L-serinate as a white solid (0.996 g, 58%).

1H NMR (400 MHz, CDCl₃) δ 8.21 (d, J=8.7 Hz, 1H), 8.05 (s, 1H), 7.94-7.85 (m, 2H), 7.39-7.30 (m, 2H), 6.19-6.01 (m, 1H), 4.84 (dt, J=8.7, 3.1 Hz, 1H), 4.49 (d, J=5.8 Hz, 2H), 4.21 (dd, J=10.1, 2.7 Hz, 1H), 3.95 (dd, J=10.1, 3.4 Hz, 1H), 3.79 (s, 3H), 3.41 (t, J=6.7 Hz, 2H), 2.28 (t, J=7.5 Hz, 2H), 1.88 (dq, J=8.1, 6.8 Hz, 2H), 1.72 (ddd, J=15.3, 7.9, 6.4 Hz, 2H), 1.56-1.45 (m, 2H), 0.92 (s, 9H), 0.08 (s, 3H), 0.06 (s, 3H).

### Methyl N-(2-(4-((6-azidohexanamido)methyl)phenyl)thiazole-4-carbonyl)-O-(tert-butyldimethylsilyl)-L-serinate (39.2)

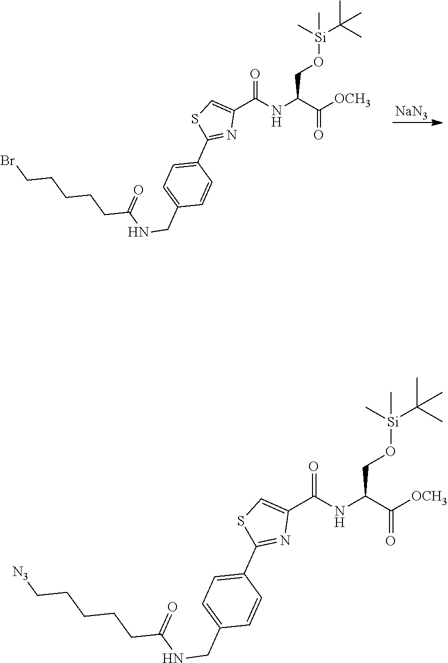 [SMILES: COC(=O)[C@H](CO[Si](C)(C)C(C)(C)C)NC(=O)c1csc(-c2ccc(CNC(=O)CCCCCBr)cc2)n1 ; COC(=O)[C@H](CO[Si](C)(C)C(C)(C)C)NC(=O)c1csc(-c2ccc(CNC(=O)CCCCCN=[N+]=[N-])cc2)n1 ; "NaN3"]

Methyl N-(2-(4-((6-bromohexanamido)methyl)phenyl)thiazole-4-carbonyl)-O-(tert-butyldimethylsilyl)-L-serinate (0.996 g, 1.59 mmole) was dissolved in DMF (3.2 mL). Sodium azide (0.123 g, 1.89 mmole) was added and the mixture was heated to 80° C. for 4½ hours, then was cooled to RT and diluted with ethyl acetate (25 mL). The mixture was washed with water (3×20 mL) and the organics were dried over magnesium sulfate, filtered, and concentrated. The crude residue was purified by silica gel chromatography to provide methyl N-(2-(4-((6-azidohexanamido)methyl)phenyl)thiazole-4-carbonyl)-O-(tert-butyldimethylsilyl)-L-serinate as a white solid (0.634 g, 68%).

¹H NMR (400 MHz, CDCl₃): δ 8.21 (d, J=8.7 Hz, 1H), 8.08 (s, 1H), 7.97-7.89 (m, 2H), 7.39-7.32 (m, 2H), 5.87 (s, 1H), 4.86 (dt, J=8.7, 2.9 Hz, 1H), 4.50 (d, J=5.8 Hz, 2H), 4.21 (dd, J=10.0, 2.7 Hz, 1H), 3.99-3.91 (m, 1H), 3.79 (s, 3H), 3.28 (t, J=6.8 Hz, 2H), 2.27 (t, J=7.5 Hz, 2H), 1.73 (tt, J=8.2, 6.5 Hz, 2H), 1.68-1.58 (m, 2H), 1.50-1.39 (m, 2H), 0.92 (s, 9H), 0.08 (s, 3H), 0.06 (s, 3H).

### Methyl O-acetyl-N—(N-(2-(4-((6-azidohexanamido)methyl)phenyl)thiazole-4-carbonyl)-O-(tert-butyldimethylsilyl)-L-seryl)-L-serinate (39.3)

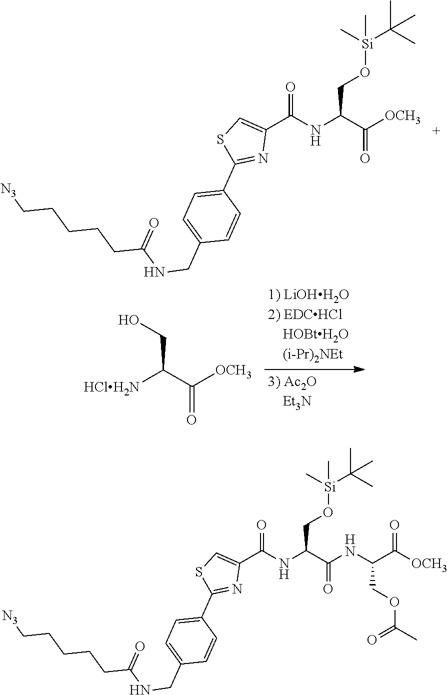

Methyl N-(2-(4-((6-azidohexanamido)methyl)phenyl)thiazole-4-carbonyl)-O-(tert-butyldimethylsilyl)-L-serinate (0.634 g, 1.08 mmole) was dissolved in 4/1/1 THF/methanol/water (2.2 mL). Lithium hydroxide monohydrate (0.070 g, 1.67 mmole) was added and the mixture was stirred at RT for 2 hours. Water (25 mL) was added and the solution was treated with 1N aq. HCl to pH=4. The mixture was extracted with ethyl acetate (3×10 mL) and the combined organics were dried over magnesium sulfate, filtered, and concentrated. The residue was dissolved in DCM (4.4 mL). L-Serine methyl ester hydrochloride (0.202 g, 1.30 mmole) was added followed by N,N-diisopropylethylamine (0.380 mL, 2.18 mmole), HOBt·H₂O (0.199 g, 1.30 mmole), and EDC·HCl (0.250 g, 1.30 mmole). The mixture was stirred at RT for 18 hours and water (25 mL) was added. The mixture was extracted with DCM (3×10 mL) and the combined organics were dried over magnesium sulfate, filtered, and concentrated. The residue was dissolved in DCM (4.4 mL) and triethylamine (0.170 mL, 1.21 mmole) and acetic anhydride (0.155 mL, 1.22 mmole) were added. The mixture was stirred at RT for 18 hours and water (25 mL) was added. The mixture was extracted with DCM (3×10 mL) and the combined organics were dried over magnesium sulfate, filtered, and concentrated. The crude residue was purified by silica gel chromatography to provide methyl O-acetyl-N—(N-(2-(4-((6-azidohexanamido)methyl)phenyl)thiazole-4-carbonyl)-O-(tert-butyldimethylsilyl)-L-seryl)-L-serinate as a colorless oil (0.538 g, 69%).

¹H NMR (400 MHz, CDCl₃): δ 8.27 (d, J=7.3 Hz, 1H), 8.12 (d, J=2.4 Hz, 1H), 7.96 (dd, J=8.2, 1.6 Hz, 2H), 7.49 (d, J=7.8 Hz, 1H), 7.40-7.33 (m, 2H), 5.87 (t, J=5.8 Hz, 1H), 4.91 (tt, J=7.2, 3.8 Hz, 1H), 4.68 (td, J=7.3, 3.8 Hz, 1H), 4.51 (ddt, J=10.5, 6.6, 3.5 Hz, 3H), 4.36 (dd, J=11.4, 3.6 Hz, 1H), 4.24 (ddd, J=9.8, 3.8, 2.8 Hz, 1H), 3.87-3.80 (m, 1H), 3.79 (s, 3H), 3.30 (t, J=6.8 Hz, 2H), 2.29 (t, J=7.5 Hz, 2H), 2.03 (s, 3H), 1.74 (tt, J=8.3, 6.5 Hz, 2H), 1.69-1.59 (m, 2H), 1.51-1.41 (m, 2H), 0.98 (s, 9H), 0.19 (s, 3H), 0.17 (s, 3H).

### Methyl 2-(2-(2-(4-((6-azidohexanamido)methyl)phenyl)thiazole-4-carboxamido)acrylamido)acrylate (39)

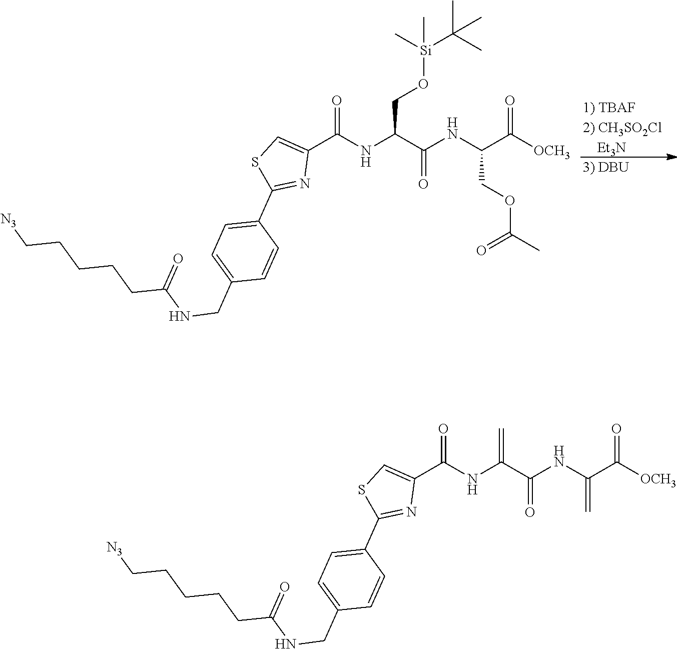 [SMILES: COC(=O)[C@H](COC(C)=O)NC(=O)[C@H](CO[Si](C)(C)C(C)(C)C)NC(=O)c1csc(-c2ccc(CNC(=O)CCCCCN=[N+]=[N-])cc2)n1 ; C=C(NC(=O)c1csc(-c2ccc(CNC(=O)CCCCCN=[N+]=[N-])cc2)n1)C(=O)NC(=C)C(=O)OC]

Methyl O-acetyl-N—(N-(2-(4-((6-azidohexanamido)methyl)phenyl)thiazole-4-carbonyl)-O-(tert-butyldimethylsilyl)-L-seryl)-L-serinate (0.538 g, 0.749 mmole) was dissolved in THF (3 mL). TBAF (1M solution in THF, 1.90 mL, 1.90 mmole) was added and the solution was stirred at RT for 2 hours. Water (25 mL) was added and the mixture was extracted with ethyl acetate (3×10 mL). The combined organics were dried over magnesium sulfate, filtered, and concentrated. The residue was dissolved in DCM (3 mL) and cooled to 0° C. Triethylamine (0.160 mL, 1.14 mmole) and methanesulfonyl chloride (0.087 mL, 1.12 mmole) were added and the mixture was stirred at 0° for 2 hours. Water (25 mL) was added and the mixture was extracted with DCM (3×10 mL). The combined organics were dried over magnesium sulfate, filtered, and concentrated. The residue was dissolved in THF (3 mL) and cooled to 0° C. DBU (0.170 mL, 1.14 mmole) was added and the mixture was stirred at 0° for 30 minutes. Water (25 mL) was added and the mixture was extracted with ethyl acetate (3×10 mL). The combined organics were dried over magnesium sulfate, filtered, and concentrated. The crude residue was purified by silica gel chromatography to provide methyl 2-(2-(2-(4-((6-azidohexanamido)methyl)phenyl)thiazole-4-carboxamido)acrylamido)acrylate as a white solid (0.160 g, 41%).

¹H NMR (400 MHz, CDCl₃): δ 10.09-9.96 (m, 1H), 8.57 (s, 1H), 8.16 (s, 1H), 8.04-7.94 (m, 2H), 7.44-7.34 (m, 2H), 6.79 (s, 1H), 6.72 (s, 1H), 6.05 (s, 1H), 5.90 (s, 1H), 5.52 (s, 1H), 4.52 (d, J=5.9 Hz, 2H), 3.92 (s, 3H), 3.30 (t, J=6.8 Hz, 2H), 2.29 (t, J=7.5 Hz, 2H), 1.80-1.69 (m, 2H), 1.68-1.59 (m, 2H), 1.43 (t, J=7.2 Hz, 2H).

### Compound 40: Methyl 2-(2-(2-(4-((6-chlorohexanamido)methyl)piperidin-1-yl)thiazole-4-carboxamido)acrylamido)acrylate

### Ethyl 2-(4-(((tert-butoxycarbonyl)amino)methyl)piperidin-1-yl)thiazole-4-carboxylate (40.1)

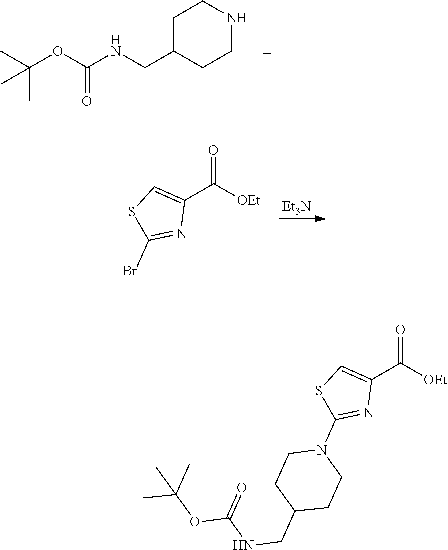 [SMILES: "Et3N" ; CC(C)(C)OC(=O)NCC1CCNCC1 ; CCOC(=O)c1csc(Br)n1 ; CCOC(=O)c1csc(N2CCC(CNC(=O)OC(C)(C)C)CC2)n1]

Tert-butyl (piperidin-4-ylmethyl)carbamate (2.500 g, 11.7 mmole) was dissolved in DMA (12 mL). Ethyl 2-bromothiazole-4-carboxylate (3.027 g, 12.8 mmole) and triethylamine (2.00 mL, 14.3 mmole) were added and the mixture was heated to 80° C. for 20 hours, then was cooled to RT and diluted with ethyl acetate (40 mL). The mixture was washed with water (3×25 mL) and the organics were dried over magnesium sulfate, filtered, and concentrated. The crude residue was purified by silica gel chromatography to provide ethyl 2-(4-(((tert-butoxycarbonyl)amino) methyl)piperidin-1-yl)thiazole-4-carboxylate as a pale yellow oil (4.194 g).

¹H NMR (400 MHz, CDCl₃): δ 7.42 (s, 1H), 4.69 (d, J=6.4 Hz, 1H), 4.35 (q, J=7.1 Hz, 2H), 4.10-4.01 (m, 2H), 3.09-2.94 (m, 4H), 1.86-1.76 (m, 2H), 1.45 (s, 9H), 1.37 (t, J=7.1 Hz, 3H), 1.34-1.28 (m, 2H).

### Methyl (2-(4-(((tert-butoxycarbonyl)amino)methyl)piperidin-1-yl)thiazole-4-carbonyl)-L-serinate (40.2)

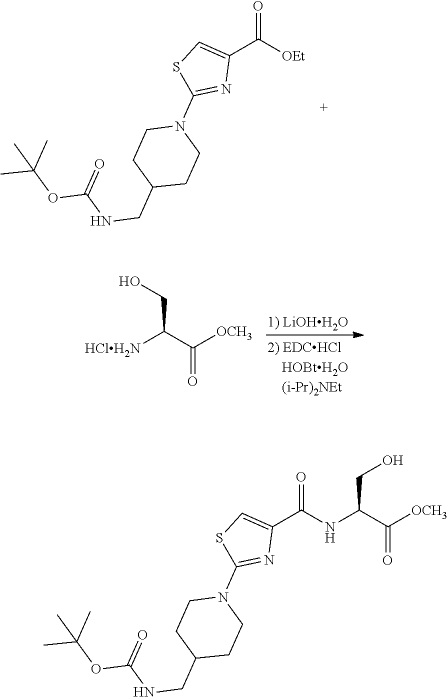

Ethyl 2-(4-(((tert-butoxycarbonyl)amino)methyl)piperidin-1-yl)thiazole-4-carboxylate (4.194 g, 11.4 mmole) was dissolved in 4/1/1 THF/methanol/water (22 mL). Lithium hydroxide monohydrate (0.715 g, 17.0 mmole) was added and the mixture was stirred at RT for 3 hours. Water (50 mL) was added and the solution was treated with 1N aq. HCl to pH=4. The mixture was extracted with ethyl acetate (3×25 mL) and the combined organics were dried over magnesium sulfate, filtered, and concentrated. The residue was dissolved in DCM (22 mL). L-Serine methyl ester hydrochloride (2.128 g, 13.7 mmole) was added followed by N,N-diisopropylethylamine (4.00 mL, 23.0 mmole), HOBt·H₂O (2.106 g, 13.8 mmole), and EDC·HCl (2.617 g, 13.7 mmole). The mixture was stirred at RT for 2½ days and water (50 mL) was added. The two layers were separated and the aqueous layer was extracted with DCM (2×10 mL). The combined organics were dried over magnesium sulfate, filtered, and concentrated and the crude residue was purified by silica gel chromatography to provide methyl (2-(4-(((tert-butoxycarbonyl)amino)methyl)piperidin-1-yl)thiazole-4-carbonyl)-L-serinate as a white solid (5.150 g, 99%).

¹H NMR (400 MHz, CDCl₃): δ 8.01 (d, J=7.4 Hz, 1H), 7.38 (s, 1H), 4.81 (dt, J=7.5, 3.8 Hz, 1H), 4.71 (s, 1H), 4.09-3.97 (m, 4H), 3.83 (s, 3H), 3.12-2.98 (m, 4H), 2.95 (t, J=6.2 Hz, 1H), 1.88-1.78 (m, 2H), 1.47 (s, 9H), 1.40-1.29 (m, 2H).

### Methyl O-(tert-butyldimethylsilyl)-N-(2-(4-((6-chlorohexanamido)methyl)piperidin-1-yl)thiazole-4-carbonyl)-L-serinate (40.3)

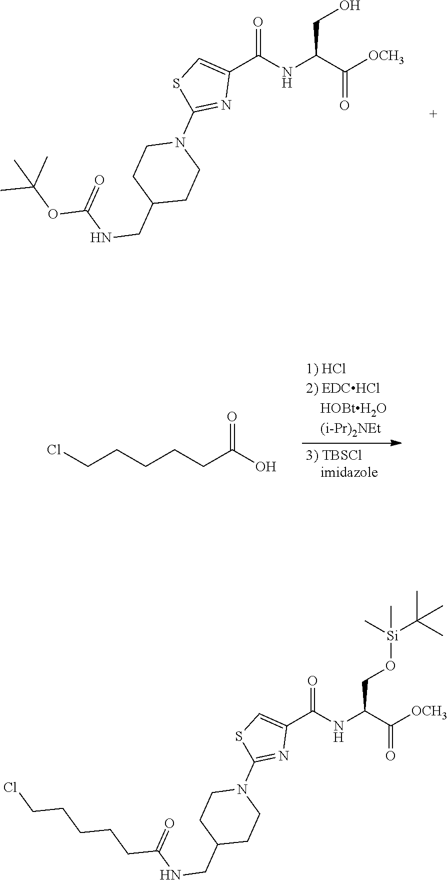 [SMILES: COC(=O)[C@H](CO)NC(=O)c1csc(N2CCC(CNC(=O)OC(C)(C)C)CC2)n1 ; O=C(O)CCCCCCl ; COC(=O)[C@H](CO[Si](C)(C)C(C)(C)C)NC(=O)c1csc(N2CCC(CNC(=O)CCCCCCl)CC2)n1]

Methyl (2-(4-(((tert-butoxycarbonyl)amino)methyl)piperidin-1-yl)thiazole-4-carbonyl)-L-serinate (0.610 g, 1.38 mmole) was dissolved in DCM (4 mL). HCl (4M solution in 1,4-dioxane, 2.70 mL, 10.8 mmole) was added and the mixture was stirred at RT for 4 hours, then was concentrated. The residue was suspended in DCM (4 mL) and 6-chlorohexanoic acid (0.251 g, 1.67 mmole) was added. N,N-Diisopropylethylamine (0.480 mL, 2.76 mmole) was added followed by HOBt·H₂O (0.253 g, 1.65 mmole) and EDC·HCl (0.315 g, 1.64 mmole), and the mixture was stirred at RT for 18 hours. Water (25 mL) was added and the mixture was extracted with DCM (3×10 mL). The combined organics were dried over magnesium sulfate, filtered, and concentrated. The residue was dissolved in DCM (4 mL). Imidazole (0.107 g, 1.57 mmole) and tert-butyldimethylchlorosilane (0.235 g, 1.56 mmole) were added and the mixture was stirred at RT for 3 days. Water (25 mL) was added and the mixture was extracted with DCM (3×10 mL). The combined organics were dried over magnesium sulfate, filtered, and concentrated. The crude residue was purified by silica gel chromatography to provide methyl O-(tert-butyldimethylsilyl)-N-(2-(4-((6-chlorohexanamido)methyl)piperidin-1-yl)thiazole-4-carbonyl)-L-serinate as a colorless oil (0.416 g, 51%).

¹H NMR (400 MHz, CDCl₃): δ 7.95 (d, J=8.8 Hz, 1H), 7.34 (s, 1H), 5.71 (t, J=6.3 Hz, 1H), 4.81-4.72 (m, 1H), 4.15 (d, J=10.2 Hz, 1H), 4.04-3.92 (m, 2H), 3.87 (dd, J=10.0, 3.4 Hz, 1H), 3.74 (s, 3H), 3.53 (t, J=6.6 Hz, 2H), 3.18 (t, J=6.2 Hz, 2H), 2.97 (tdd, J=12.7, 8.2, 2.6 Hz, 2H), 2.20 (t, J=7.5 Hz, 2H), 1.82-1.71 (m, 5H), 1.67 (tt, J=8.5, 6.5 Hz, 2H), 1.52-1.42 (m, 2H), 1.38-1.27 (m, 2H), 0.87 (s, 9H), 0.03 (s, 3H), 0.01 (s, 3H).

### Methyl O-acetyl-N—(O-(tert-butyldimethylsilyl)-N-(2-(4-((6-chlorohexanamido)methyl) piperidin-1-yl)thiazole-4-carbonyl)-L-seryl)-L-serinate (40.4)

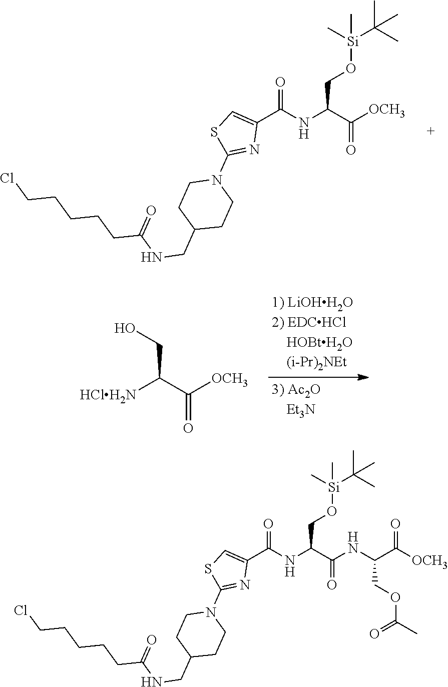

Methyl O-(tert-butyldimethylsilyl)-N-(2-(4-((6-chlorohexanamido)methyl)piperidin-1-yl)thiazole-4-carbonyl)-L-serinate (0.416 g, 0.706 mmole) was dissolved in 4/1/1 THF/methanol/water (2.8 mL). Lithium hydroxide monohydrate (0.053 g, 1.26 mmole) was added and the mixture was stirred at RT for 2 hours. Water (25 mL) was added and the solution was treated with 1N aq. HCl to pH=4. The mixture was extracted with ethyl acetate (3×10 mL) and the combined organics were dried over magnesium sulfate, filtered, and concentrated. The residue was dissolved in DCM (2.8 mL) and L-serine methyl ester hydrochloride (0.135 g, 0.868 mmole) was added. N,N-Diisopropylethylamine (0.250 mL, 1.44 mmole) was added followed by HOBt·H₂O (0.136 g, 0.888 mmole) and EDC·HCl (0.165 g, 0.861 mmole) and the mixture was stirred at RT for 18 hours. Water (25 mL) was added and the mixture was extracted with DCM (3×10 mL). The combined organics were dried over magnesium sulfate, filtered, and concentrated. The residue was dissolved in DCM (2.8 mL) and triethylamine (0.110 mL, 0.785 mmole) and acetic anhydride (0.074 mL, 0.783 mmole) were added. The solution was stirred at RT for 3 hours and water (25 mL) was added. The mixture was extracted with DCM (3×10 mL) and the combined organics were dried over magnesium sulfate, filtered, and concentrated. The crude residue was purified by silica gel chromatography to provide methyl O-acetyl-N—(O-(tert-butyldimethylsilyl)-N-(2-(4-((6-chlorohexanamido)methyl)piperidin-1-yl)thiazole-4-carbonyl)-L-seryl)-L-serinate as a thick, colorless oil (0.299 g, 59%).

¹H NMR (400 MHz, CDCl₃): δ 7.98 (d, J=7.1 Hz, 1H), 7.43 (d, J=7.8 Hz, 1H), 7.37 (d, J=2.7 Hz, 1H), 5.57 (t, J=6.1 Hz, 1H), 4.86 (dq, J=8.2, 4.0 Hz, 1H), 4.57 (ddd, J=7.2, 6.0, 3.6 Hz, 1H), 4.46 (ddd, J=11.8, 7.9, 4.0 Hz, 1H), 4.31 (dd, J=11.4, 3.7 Hz, 1H), 4.22-4.15 (m, 1H), 4.00 (d, J=13.0 Hz, 2H), 3.76 (d, J=2.3 Hz, 3H), 3.75-3.70 (m, 1H), 3.54 (t, J=6.6 Hz, 2H), 3.20 (t, J=6.2 Hz, 2H), 2.99 (tdd, J=12.8, 5.0, 2.7 Hz, 2H), 2.21 (t, J=7.5 Hz, 2H), 2.02 (d, J=2.7 Hz, 3H), 1.85-1.73 (m, 5H), 1.73-1.64 (m, 2H), 1.55-1.43 (m, 2H), 1.40-1.27 (m, 2H), 0.92 (s, 9H), 0.14 (s, 3H), 0.12 (s, 3H).

### Methyl 2-(2-(2-(4-((6-chlorohexanamido)methyl)piperidin-1-yl)thiazole-4-carboxamido)acrylamido)acrylate (40)

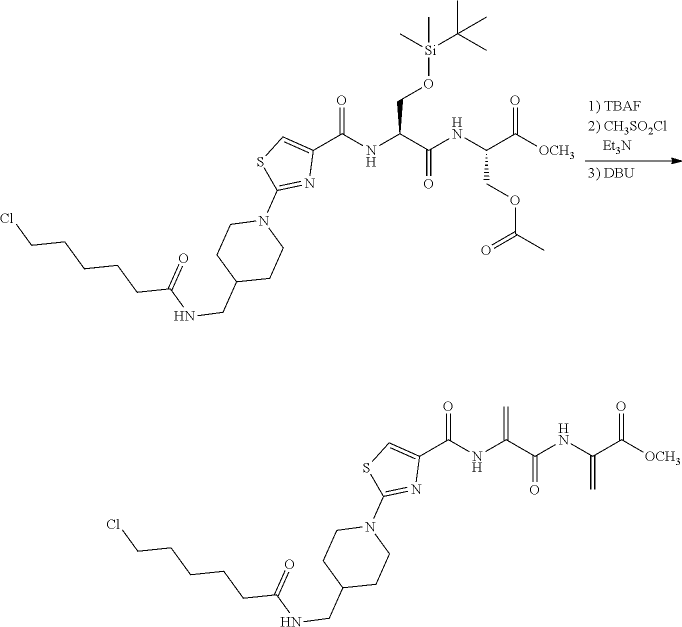 [SMILES: COC(=O)[C@H](COC(C)=O)NC(=O)[C@H](CO[Si](C)(C)C(C)(C)C)NC(=O)c1csc(N2CCC(CNC(=O)CCCCCCl)CC2)n1 ; C=C(NC(=O)c1csc(N2CCC(CNC(=O)CCCCCCl)CC2)n1)C(=O)NC(=C)C(=O)OC]

Methyl O-acetyl-N—(O-(tert-butyldimethylsilyl)-N-(2-(4-((6-chlorohexanamido)methyl) piperidin-1-yl)thiazole-4-carbonyl)-L-seryl)-L-serinate (0.299 g, 0.416 mmole) was dissolved in THF (1.6 mL). TBAF (1M solution in THF, 1.05 mL, 1.05 mmole) was added and the solution was stirred at RT for 2 hours. Water (25 mL) was added and the mixture was extracted with ethyl acetate (3×10 mL). The combined organics were dried over magnesium sulfate, filtered, and concentrated. The residue was dissolved in DCM (1.6 mL) and cooled to 0° C. Triethylamine (0.088 mL, 0.628 mmole) and methanesulfonyl chloride (0.049 mL, 0.633 mmole) were added and the mixture was stirred at 0° for 90 minutes. Water (25 mL) was added and the mixture was extracted with DCM (3×10 mL). The combined organics were dried over magnesium sulfate, filtered, and concentrated. The residue was dissolved in THF (1.6 mL) and cooled to 0° C. DBU (0.095 mL, 0.635 mmole) was added and the mixture was stirred at 0° for 60 minutes. Water (25 mL) was added and the mixture was extracted with ethyl acetate (3×10 mL). The combined organics were dried over magnesium sulfate, filtered, and concentrated. The crude residue was purified by silica gel chromatography to provide methyl 2-(2-(2-(4-((6-chlorohexanamido) methyl)piperidin-1-yl)thiazole-4-carboxamido)acrylamido)acrylate as a colorless oil (0.154 g, 70%).

¹H NMR (400 MHz, CDCl₃): δ 9.72 (s, 1H), 8.51 (s, 1H), 7.40 (s, 1H), 6.71 (d, J=2.1 Hz, 1H), 6.65 (s, 1H), 6.00 (d, J=1.3 Hz, 1H), 5.55 (t, J=6.2 Hz, 1H), 5.43 (t, J=1.9 Hz, 1H), 4.04 (d, J=12.8 Hz, 2H), 3.89 (s, 3H), 3.54 (t, J=6.6 Hz, 2H), 3.20 (t, J=6.2 Hz, 2H), 3.01 (td, J=12.6, 2.6 Hz, 2H), 2.21 (t, J=7.5 Hz, 2H), 1.79 (dtd, J=14.8, 8.2, 7.4, 3.9 Hz, 5H), 1.74-1.63 (m, 2H), 1.49 (tt, J=9.7, 5.9 Hz, 2H), 1.35 (td, J=12.2, 11.7, 4.3 Hz, 2H).

### Compound 41: Methyl 2-(2-(2-(4-((2-(2-methoxyethoxy)acetamido)methyl)piperidin-1-yl)thiazole-4-carboxamido)acrylamido)acrylate

### Methyl O-(tert-butyldimethylsilyl)-N-(2-(4-((2-(2-methoxyethoxy)acetamido)methyl)piperidin-1-yl)thiazole-4-carbonyl)-L-serinate (41.1)

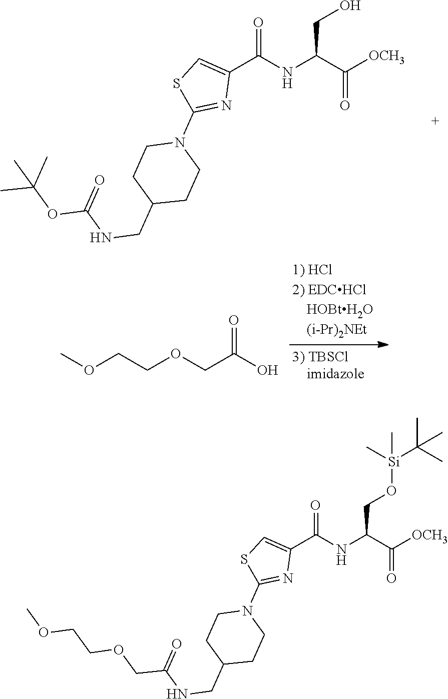

Methyl (2-(4-(((tert-butoxycarbonyl)amino)methyl)piperidin-1-yl)thiazole-4-carbonyl)-L-serinate (Example 40.2, 0.614 g, 1.39 mmole) was dissolved in DCM (2.7 mL). HCl (4M solution in 1,4-dioxane, 2.70 mL, 10.8 mmole) was added and the mixture was stirred at RT for 4 hours, then was concentrated. The residue was suspended in DCM (4 mL) and 2-(2-methoxyethoxy)acetic acid (0.235 g, 1.75 mmole) was added. N,N-Diisopropylethylamine (0.480 mL, 2.76 mmole) was added followed by HOBt·H₂O (0.252 g, 1.65 mmole) and EDC·HCl (0.319 g, 1.66 mmole), and the mixture was stirred at RT for 18 hours. Water (25 mL) was added and the mixture was extracted with DCM (3×10 mL). The combined organics were dried over magnesium sulfate, filtered, and concentrated. The residue was dissolved in DCM (2.7 mL). Imidazole (0.104 g, 1.53 mmole) and tert-butyldimethylchlorosilane (0.226 g, 1.50 mmole) were added and the mixture was stirred at RT for 18 hours. Water (25 mL) was added and the mixture was extracted with DCM (3×10 mL). The combined organics were dried over magnesium sulfate, filtered, and concentrated. The crude residue was purified by silica gel chromatography to provide methyl O-(tert-butyldimethylsilyl)-N-(2-(4-((2-(2-methoxyethoxy)acetamido)methyl)piperidin-1-yl)thiazole-4-carbonyl)-L-serinate as a thick, colorless gel (0.363 g, 46%).

¹H NMR (400 MHz, CDCl₃): δ 7.96 (d, J=8.8 Hz, 1H), 7.36 (s, 1H), 7.17 (t, J=6.2 Hz, 1H), 4.83-4.73 (m, 1H), 4.15 (dd, J=10.0, 2.6 Hz, 1H), 4.01 (s, 4H), 3.88 (dd, J=10.0, 3.4 Hz, 1H), 3.75 (s, 3H), 3.72-3.65 (m, 2H), 3.61-3.52 (m, 2H), 3.41 (s, 3H), 3.24 (t, J=6.3 Hz, 2H), 2.99 (tdd, J=12.5, 7.1, 2.6 Hz, 2H), 1.87-1.70 (m, 3H), 1.36 (dp, J=18.8, 7.2, 6.1 Hz, 2H), 0.88 (s, 9H), 0.04 (s, 3H), 0.03 (s, 3H).

### Methyl O-acetyl-N—(O-(tert-butyldimethylsilyl)-N-(2-(4-((2-(2-methoxyethoxy)acetamido)methyl)piperidin-1-yl)thiazole-4-carbonyl)-L-seryl)-L-serinate (41.2)

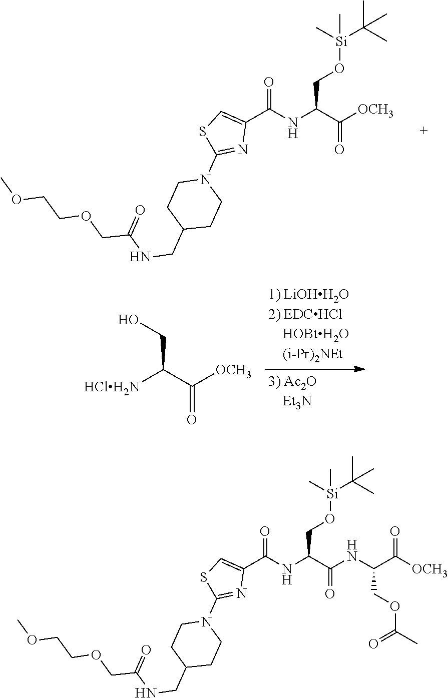

Methyl O-(tert-butyldimethylsilyl)-N-(2-(4-((2-(2-methoxyethoxy)acetamido)methyl)piperidin-1-yl)thiazole-4-carbonyl)-L-serinate (0.363 g, 0.634 mmole) was dissolved in 4/1/1 THF/methanol/water (2.6 mL). Lithium hydroxide monohydrate (0.043 g, 1.02 mmole) was added and the mixture was stirred at RT for 2 hours. Water (25 mL) was added and the solution was treated with 1N aq. HCl to pH=4. The mixture was extracted with ethyl acetate (3×10 mL) and the combined organics were dried over magnesium sulfate, filtered, and concentrated. The residue was dissolved in DCM (2.6 mL) and L-serine methyl ester hydrochloride (0.125 g, 0.803 mmole) was added. N,N-Diisopropylethylamine (0.220 mL, 1.26 mmole) was added followed by HOBt·H₂O (0.122 g, 0.797 mmole) and EDC·HCl (0.149 g, 0.777 mmole) and the mixture was stirred at RT for 18 hours. Water (25 mL) was added and the mixture was extracted with DCM (3×10 mL). The combined organics were dried over magnesium sulfate, filtered, and concentrated. The residue was dissolved in DCM (2.6 mL) and triethylamine (0.100 mL, 0.714 mmole) and acetic anhydride (0.066 mL, 0.698 mmole) were added. The solution was stirred at RT for 3 hours and water (25 mL) was added. The mixture was extracted with DCM (3×10 mL) and the combined organics were dried over magnesium sulfate, filtered, and concentrated. The crude residue was purified by silica gel chromatography to provide methyl O-acetyl-N—(O-(tert-butyldimethylsilyl)-N-(2-(4-((2-(2-methoxyethoxy)acetamido)methyl)piperidin-1-yl)thiazole-4-carbonyl)-L-seryl)-L-serinate as a thick, colorless gel (0.231 g, 52%).

¹H NMR (400 MHz, CDCl₃): δ 7.99 (d, J=7.1 Hz, 1H), 7.43 (d, J=7.8 Hz, 1H), 7.37 (d, J=2.8 Hz, 1H), 7.17 (t, J=6.1 Hz, 1H), 4.87 (dt, J=7.7, 3.8 Hz, 1H), 4.58 (td, J=7.2, 3.7 Hz, 1H), 4.52-4.43 (m, 1H), 4.31 (dd, J=11.4, 3.7 Hz, 1H), 4.18 (ddd, J=9.8, 3.7, 1.7 Hz, 1H), 4.06-3.97 (m, 2H), 4.01 (s, 2H), 3.76 (s, 3H), 3.75-3.71 (m, 1H), 3.71-3.65 (m, 2H), 3.61-3.53 (m, 2H), 3.41 (s, 3H), 3.24 (t, J=6.3 Hz, 2H), 3.07-2.93 (m, 2H), 2.02 (d, J=2.5 Hz, 3H), 1.88-1.71 (m, 3H), 1.36 (p, J=12.4, 10.6 Hz, 2H), 0.92 (s, 9H), 0.14 (s, 3H), 0.12 (s, 3H).

### Methyl 2-(2-(2-(4-((2-(2-methoxyethoxy)acetamido)methyl)piperidin-1-yl)thiazole-4-carboxamido)acrylamido)acrylate (41)

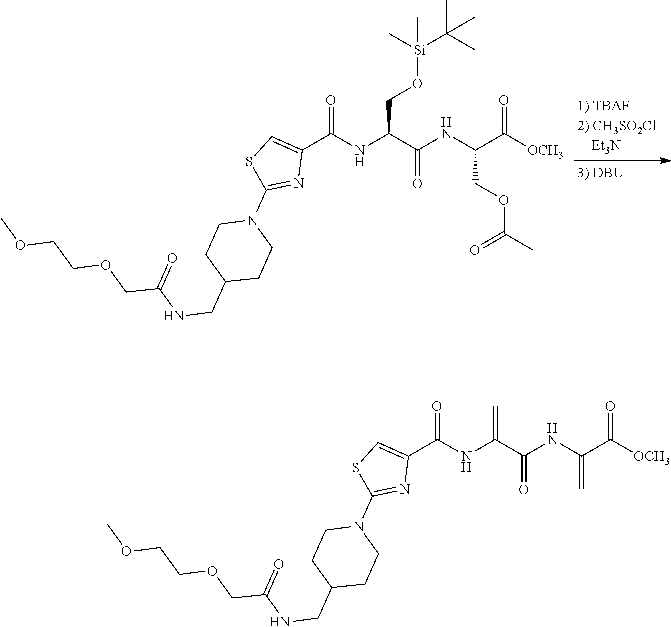 [SMILES: COCCOCC(=O)NCC1CCN(c2nc(C(=O)N[C@@H](CO[Si](C)(C)C(C)(C)C)C(=O)N[C@@H](COC(C)=O)C(=O)OC)cs2)CC1 ; C=C(NC(=O)c1csc(N2CCC(CNC(=O)COCCOC)CC2)n1)C(=O)NC(=C)C(=O)OC]

Methyl O-acetyl-N—(O-(tert-butyldimethylsilyl)-N-(2-(4-((2-(2-methoxyethoxy)acetamido)methyl)piperidin-1-yl)thiazole-4-carbonyl)-L-seryl)-L-serinate (0.231 g, 0.329 mmole) was dissolved in THF (1.2 mL). TBAF (1M solution in THF, 0.820 mL, 0.820 mmole) was added and the solution was stirred at RT for 2 hours. Water (25 mL) was added and the mixture was extracted with ethyl acetate (3×10 mL). The combined organics were dried over magnesium sulfate, filtered, and concentrated. The residue was dissolved in DCM (1.2 mL) and cooled to 0° C. Triethylamine (0.070 mL, 0.499 mmole) and methanesulfonyl chloride (0.039 mL, 0.504 mmole) were added and the mixture was stirred at 0° for 90 minutes. Water (25 mL) was added and the mixture was extracted with DCM (3×10 mL). The combined organics were dried over magnesium sulfate, filtered, and concentrated. The residue was dissolved in THF (1.2 mL) and cooled to 0° C. DBU (0.074 mL, 0.495 mmole) was added and the mixture was stirred at 0° for 60 minutes. Water (25 mL) was added and the mixture was extracted with ethyl acetate (3×10 mL). The combined organics were dried over magnesium sulfate, filtered, and concentrated. The crude residue was purified by silica gel chromatography to provide methyl 2-(2-(2-(4-((2-(2-methoxyethoxy)acetamido)methyl)piperidin-1-yl)thiazole-4-carboxamido)acrylamido)acrylate as a colorless oil (0.154 g).

¹H NMR (400 MHz, CDCl₃): δ 9.75 (s, 1H), 8.53 (s, 1H), 7.43 (s, 1H), 7.20 (d, J=6.4 Hz, 1H), 6.74 (d, J=2.1 Hz, 1H), 6.68 (s, 1H), 6.02 (d, J=1.3 Hz, 1H), 5.45 (t, J=1.9 Hz, 1H), 4.07 (d, J=12.9 Hz, 2H), 4.04 (s, 2H), 3.91 (s, 3H), 3.75-3.67 (m, 2H), 3.63-3.56 (m, 2H), 3.44 (s, 3H), 3.27 (t, J=6.4 Hz, 2H), 3.04 (td, J=12.6, 2.7 Hz, 2H), 1.88 (d, J=18.9 Hz, 2H), 1.83-1.73 (m, 1H), 1.46-1.33 (m, 2H).

### Compound 42: Methyl 2-(2-(2-(4-((((3-methoxypropyl)carbamoyl)oxy)methyl)phenyl)thiazole-4-carboxamido)acrylamido)acrylate

### 4-Cyanobenzyl (3-methoxypropyl)carbamate (42.1)

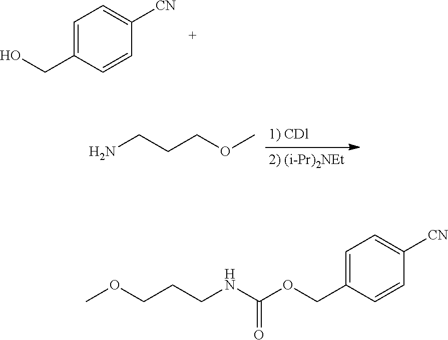

4-(Hydroxymethyl)benzonitrile (1.504 g, 11.3 mmole) was dissolved in THF (22 mL). N,N′-Carbonyldiimidazole (2.177 g, 13.4 mmole) was added and the solution was stirred at RT for 18 hours. Water (50 mL) was added and the mixture was extracted with ethyl acetate (3×15 mL). The combined organics were washed sequentially with water (2×25 mL) and sat. aq. sodium chloride (1×25 mL), then were dried over magnesium sulfate, filtered, and concentrated. The residue was dissolved in DMF (22 mL). 3-Methoxypropan-1-amine (1.30 mL, 12.7 mmole) and N,N-diisopropylethylamine (2.15 mL, 12.3 mmole) were added and the solution was heated to 70° C. for 3 hours. The mixture was cooled to RT and water (50 mL) was added. The mixture was extracted with ethyl acetate (3×15 mL) and the combined organics were washed sequentially with water (2×25 mL) and sat. aq. sodium chloride (1×25 mL). The organics were dried over magnesium sulfate, filtered and concentrated to provide 4-cyanobenzyl (3-methoxypropyl)carbamate as a pale yellow oil (2.774 g, 99%).

¹H NMR (400 MHz, CDCl₃): δ 7.71-7.61 (m, 2H), 7.46 (d, J=8.0 Hz, 2H), 5.30 (s, 1H), 5.15 (s, 2H), 3.48 (t, J=5.8 Hz, 2H), 3.39-3.28 (m, 5H), 1.80 (p, J=6.0 Hz, 2H).

### 4-Carbamothioylbenzyl (3-methoxypropyl)carbamate (42.2)

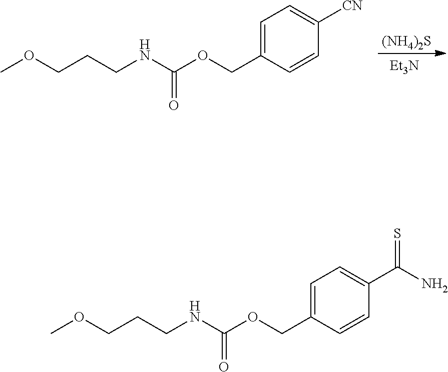 [SMILES: "Et3N" ; COCCCNC(=O)OCc1ccc(C#N)cc1 ; "(NH4)2S" ; COCCCNC(=O)OCc1ccc(C(N)=S)cc1]

4-Cyanobenzyl (3-methoxypropyl)carbamate (2.774 g, 11.2 mmole) was dissolved in pyridine (11 mL). Triethylamine (1.70 mL, 12.1 mmole) and ammonium sulfide (40% aqueous solution, 2.30 mL, 13.5 mmole) were added and the mixture was warmed to 50° C. for 90 minutes, then was cooled to RT and concentrated. The residue was dissolved in ethyl acetate (15 mL) and water (25 mL) and 1N aq. HCl (20 mL) were added. The two layers were separated and the aqueous layer was extracted with ethyl acetate (2×15 mL). The combined organics were dried over magnesium sulfate, filtered, and concentrated to provide 4-carbamothioylbenzyl (3-methoxypropyl)carbamate as a yellow solid (3.371 g).

¹H NMR (400 MHz, CDCl₃): δ 9.86 (s, 1H), 9.49 (s, 1H), 7.93-7.83 (m, 2H), 7.40-7.32 (m, 2H), 7.29 (t, J=5.7 Hz, 1H), 5.05 (s, 2H), 3.31 (t, J=6.3 Hz, 2H), 3.21 (s, 3H), 3.04 (q, J=6.8 Hz, 2H), 1.63 (p, J=6.6 Hz, 2H).

### Ethyl 2-(4-((((3-methoxypropyl)carbamoyl)oxy)methyl)phenyl)thiazole-4-carboxylate (42.3)

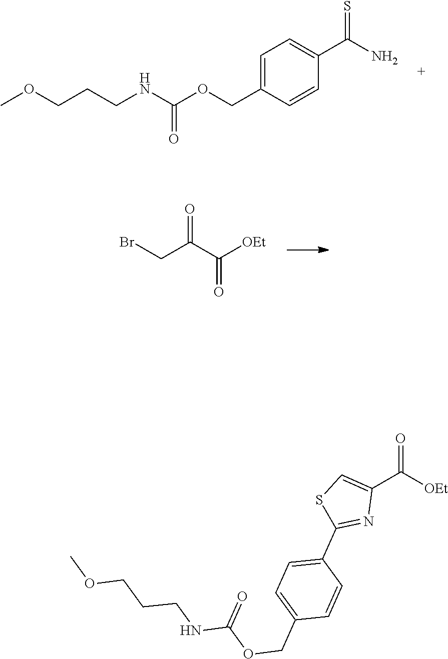 [SMILES: COCCCNC(=O)OCc1ccc(C(N)=S)cc1 ; CCOC(=O)C(=O)CBr ; CCOC(=O)c1csc(-c2ccc(COC(=O)NCCCOC)cc2)n1]

4-Carbamothioylbenzyl (3-methoxypropyl)carbamate (3.371 g, 11.9 mmole) was dissolved in ethanol (24 mL). Ethyl bromopyruvate (1.80 mL, 14.3 mmole) was added and the mixture was heated to 80° C. for 3 hours, then was cooled to RT and concentrated. The crude residue was purified by silica gel chromatography to provide ethyl 2-(4-((((3-methoxypropyl)carbamoyl)oxy)methyl)phenyl)thiazole-4-carboxylate as a yellow solid (1.435 g, 32%).

¹H NMR (400 MHz, CDCl₃): δ 8.15 (s, 1H), 8.03-7.92 (m, 2H), 7.43 (d, J=8.2 Hz, 2H), 5.21 (s, 1H), 5.12 (s, 2H), 4.44 (q, J=7.1 Hz, 2H), 3.45 (t, J=5.9 Hz, 2H), 3.36-3.27 (m, 2H), 3.32 (s, 3H), 1.79 (q, J=6.1 Hz, 2H), 1.42 (t, J=7.1 Hz, 3H).

### Methyl O-(tert-butyldimethylsilyl)-N-(2-(4-((((3-methoxypropyl)carbamoyl)oxy)methyl)phenyl)thiazole-4-carbonyl)-L-serinate (42.4)

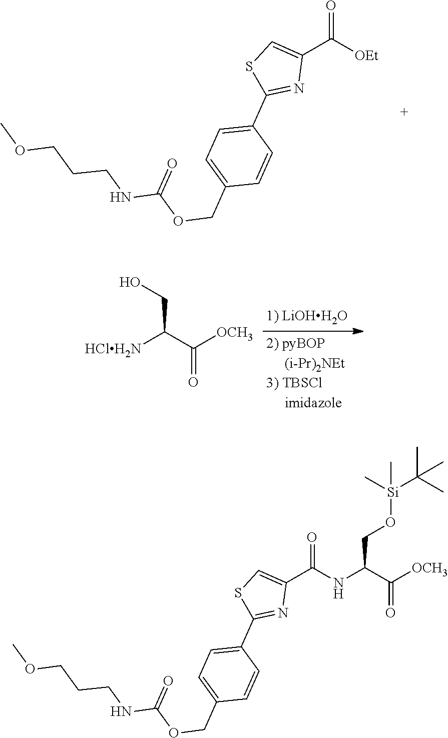

Ethyl 2-(4-((((3-methoxypropyl)carbamoyl)oxy)methyl)phenyl)thiazole-4-carboxylate (0.702 g, 1.85 mmole) was dissolved in 4/1/1 THF/methanol/water (3.6 mL). Lithium hydroxide monohydrate (0.118 g, 2.81 mmole) was added and the mixture was stirred at RT for 3 hours. Water (25 mL) was added and the solution was treated with 1N aq. HCl to pH=4. The mixture was extracted with ethyl acetate (3×20 mL) and the combined organics were dried over magnesium sulfate, filtered and concentrated. The residue was suspended in DCM (7.2 mL) and L-serine methyl ester hydrochloride (0.357 g, 2.29 mmole) was added. N,N-diisopropylethylamine (0.650 mL, 3.73 mmole) and pyBOP (1.064 g, 2.04 mmole) were added and the mixture was stirred at RT for 18 hours. Water (25 mL) was added and the mixture was extracted with DCM (3×10 mL). The combined organics were dried over magnesium sulfate, filtered, and concentrated. The residue was dissolved in DCM (7.2 mL) and imidazole (0.145 g, 2.13 mmole) and tert-butyldimethylchlorosilane (0.317 g, 2.10 mmole) were added. The mixture was stirred at RT for 18 hours and water (25 mL) was added. The mixture was extracted with DCM (3×10 mL) and the combined organics were dried over magnesium sulfate, filtered, and concentrated. The crude residue was purified by silica gel chromatography to provide methyl O-(tert-butyldimethylsilyl)-N-(2-(4-((((3-methoxypropyl)carbamoyl)oxy)methyl)phenyl)thiazole-4-carbonyl)-L-serinate as a yellow oil (0.842 g, 80%).

¹H NMR (400 MHz, CDCl₃): δ 8.22 (d, J=8.7 Hz, 1H), 8.10 (s, 1H), 7.98-7.90 (m, 2H), 7.47-7.39 (m, 2H), 5.21 (d, J=20.6 Hz, 1H), 5.13 (s, 2H), 4.85 (dt, J=8.7, 3.0 Hz, 1H), 4.20 (dd, J=10.1, 2.6 Hz, 1H), 3.94 (dd, J=10.1, 3.4 Hz, 1H), 3.78 (s, 3H), 3.46 (t, J=5.8 Hz, 2H), 3.32 (d, J=4.8 Hz, 5H), 1.83-1.72 (m, 2H), 0.91 (s, 9H), 0.07 (s, 3H), 0.06 (s, 3H).

### Methyl O-acetyl-N—(O-(tert-butyldimethylsilyl)-N-(2-(4-((((3-methoxypropyl)carbamoyl)oxy)methyl)phenyl)thiazole-4-carbonyl)-L-seryl)-L-serinate (42.5)

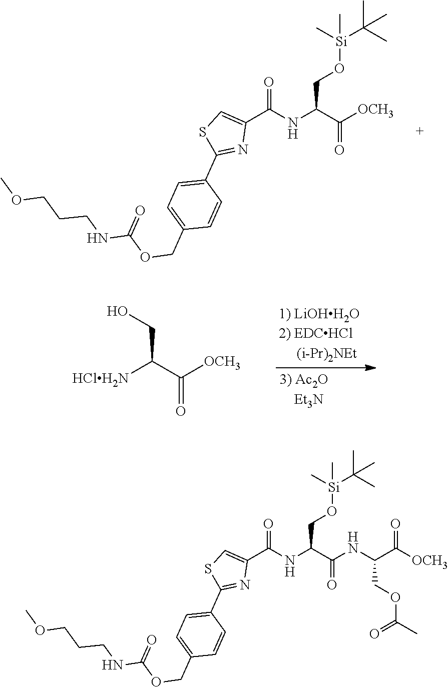

Methyl O-(tert-butyldimethylsilyl)-N-(2-(4-((((3-methoxypropyl)carbamoyl)oxy)methyl)phenyl)thiazole-4-carbonyl)-L-serinate (0.842 g, 1.49 mmole) was dissolved in 4/1/1 THF/methanol/water (3 mL). Lithium hydroxide monohydrate (0.131 g, 3.12 mmole) was added and the mixture was stirred at RT for 2 hours. Water (25 mL) was added and the solution was treated with 1N aq. HCl to pH=4. The mixture was extracted with ethyl acetate (3×10 mL) and the combined organics were dried over magnesium sulfate, filtered, and concentrated. The residue was dissolved in DCM (3 mL) and L-serine methyl ester hydrochloride (0.284 g, 1.83 mmole) was added, followed by N,N-diisopropylethylamine (0.520 mL, 2.99 mmole) and EDC·HCl (0.342 g, 1.78 mmole). The mixture was stirred at RT for 18 hours and water (25 mL) was added. The mixture was extracted with DCM (3×10 mL) and the combined organics were dried over magnesium sulfate, filtered, and concentrated. The residue was dissolved in DCM (3 mL) and triethylamine (0.230 ml, 1.64 mmole) and acetic anhydride (0.170 mL, 1.80 mmole) were added. The mixture was stirred at RT for 20 hours and water (25 mL) was added. The mixture was extracted with DCM (3×10 mL) and the combined organics were dried over magnesium sulfate, filtered, and concentrated. The crude residue was purified by silica gel chromatography to provide methyl O-acetyl-N—(O-(tert-butyldimethylsilyl)-N-(2-(4-((((3-methoxypropyl)carbamoyl)oxy)methyl)phenyl)thiazole-4-carbonyl)-L-seryl)-L-serinate as a colorless oil (0.693 g, 67%).

¹H NMR (400 MHz, CDCl₃): δ 8.30-8.21 (m, 1H), 8.11 (d, J=2.5 Hz, 1H), 7.95 (dq, J=8.6, 1.9 Hz, 2H), 7.44 (dd, J=11.6, 7.9 Hz, 3H), 5.22 (s, 1H), 5.14 (s, 2H), 4.89 (dq, J=7.2, 3.6 Hz, 1H), 4.66 (td, J=7.3, 3.7 Hz, 1H), 4.48 (ddd, J=11.1, 7.1, 4.0 Hz, 1H), 4.33 (dd, J=11.4, 3.6 Hz, 1H), 4.22 (dt, J=9.9, 3.6 Hz, 1H), 3.84-3.78 (m, 1H), 3.76 (d, J=4.0 Hz, 3H), 3.46 (t, J=5.8 Hz, 2H), 3.32 (d, J=5.1 Hz, 5H), 2.00 (s, 3H), 1.79 (p, J=6.0 Hz, 2H), 0.95 (s, 9H), 0.18-0.12 (m, 6H).

### Methyl 2-(2-(2-(4-((((3-methoxypropyl)carbamoyl)oxy)methyl)phenyl)thiazole-4-carboxamido)acrylamido)acrylate (42)

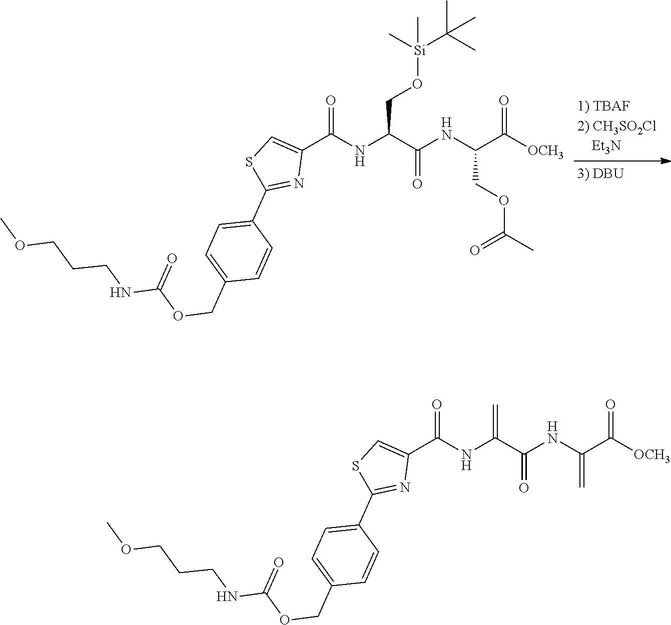 [SMILES: COCCCNC(=O)OCc1ccc(-c2nc(C(=O)N[C@@H](CO[Si](C)(C)C(C)(C)C)C(=O)N[C@@H](COC(C)=O)C(=O)OC)cs2)cc1 ; C=C(NC(=O)c1csc(-c2ccc(COC(=O)NCCCOC)cc2)n1)C(=O)NC(=C)C(=O)OC]

Methyl O-acetyl-N—(O-(tert-butyldimethylsilyl)-N-(2-(4-((((3-methoxypropyl)carbamoyl)oxy)methyl)phenyl)thiazole-4-carbonyl)-L-seryl)-L-serinate (0.693 g, 0.997 mmole) was dissolved in THF (2 mL). TBAF (1M solution in THF, 2.50 mL, 2.50 mmole) was added and the mixture was stirred at RT for 3 hours. Water (25 mL) was added and the mixture was extracted with ethyl acetate (3×10 mL). The combined organics were dried over magnesium sulfate, filtered and concentrated. The residue was dissolved in DCM (2 mL) and cooled to 0° C. Triethylamine (0.210 mL, 1.50 mmole) and methanesulfonyl chloride (0.115 mL, 1.49 mmole) were added and the mixture was stirred at 0° for 45 minutes. Water (25 mL) was added and the mixture was extracted with DCM (3×10 mL). The combined organics were dried over magnesium sulfate, filtered and concentrated. The residue was dissolved in THF (2 mL) and cooled to 0° C. DBU (0.220 mL, 1.47 mmole) was added and the mixture was stirred at 0° C. for 45 minutes. Water (25 mL) was added and the mixture was extracted with ethyl acetate (3×10 mL). The combined organics were dried over magnesium sulfate, filtered and concentrated. The crude residue was purified by silica gel chromatography to provide methyl 2-(2-(2-(4-((((3-methoxypropyl)carbamoyl)oxy)methyl)phenyl)thiazole-4-carboxamido)acrylamido)acrylate as a white solid (0.193 g, 39%).

¹H NMR (400 MHz, CDCl₃): δ 10.02 (s, 1H), 8.55 (s, 1H), 8.15 (s, 1H), 8.05-7.93 (m, 2H), 7.51-7.41 (m, 2H), 6.78 (d, J=2.2 Hz, 1H), 6.71 (s, 1H), 6.03 (d, J=1.4 Hz, 1H), 5.50 (t, J=1.9 Hz, 1H), 5.21 (s, 1H), 5.14 (s, 2H), 3.90 (s, 3H), 3.47 (t, J=5.8 Hz, 2H), 3.33 (d, J=5.5 Hz, 5H), 1.79 (p, J=6.1 Hz, 2H).

### Compound 43: Methyl 2-(2-(2-(3-(((tert-butoxycarbonyl)amino)methyl)phenyl)thiazole-4-carboxamido)acrylamido)acrylate

### 3-((1,3-Dioxoisoindolin-2-yl)methyl)benzonitrile (43.1)

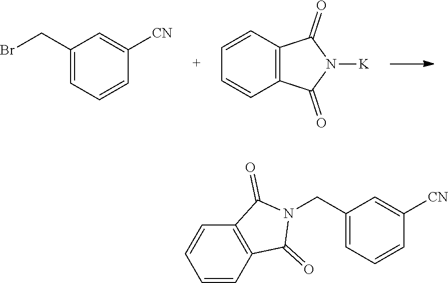

3-Cyanobenzyl bromide (2.003 g, 10.2 mmole) was dissolved in DMF (10 mL) and potassium phthalimide (2.084 g, 11.3 mmole) was added. The mixture was stirred at RT for 4 hours and water (50 mL) was added. The precipitated solid was collected by filtration, washed with water, and dried under vacuum to provide 3-((1,3-dioxoisoindolin-2-yl)methyl)benzonitrile as a white solid (2.511 g, 94%).

¹H NMR (400 MHz, CDCl₃): δ 7.87 (dd, J=5.5, 3.1 Hz, 2H), 7.79-7.69 (m, 3H), 7.67 (dt, J=7.8, 1.5 Hz, 1H), 7.57 (dt, J=7.7, 1.4 Hz, 1H), 7.44 (t, J=7.8 Hz, 1H), 4.86 (s, 2H).

### 3-((1,3-Dioxoisoindolin-2-yl)methyl)benzothioamide (43.2)

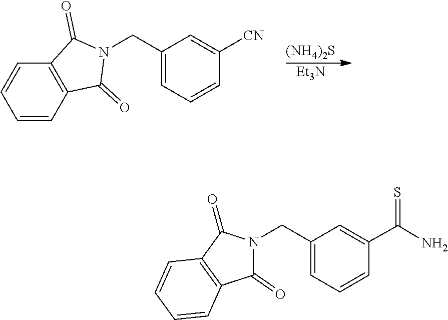

3-((1,3-Dioxoisoindolin-2-yl)methyl)benzonitrile (2.511 g, 9.57 mmole) was dissolved in pyridine. Triethylamine (1.50 mL, 10.7 mmole) and ammonium sulfide (40% aqueous solution, 2.00 mL, 11.7 mmole) were added and the mixture was warmed to 50° C. for 60 minutes, then was cooled to RT and concentrated. The residue was washed with 0.5N aq. HCl (40 mL) and the solid was collected by filtration, washed with water and diethyl ether, and allowed to air dry to provide 3-((1,3-dioxoisoindolin-2-yl)methyl)benzothioamide as a yellow solid (3.145 g).

¹H NMR (400 MHz, d₆-DMSO): δ 9.82 (s, 1H), 9.51 (s, 1H), 8.59 (m, 1H), 7.91-7.83 (m, 3H), 7.79-7.74 (m, 2H), 7.48-7.30 (m, 2H), 4.78 (s, 2H).

### Ethyl 2-(3-((1,3-dioxoisoindolin-2-yl)methyl)phenyl)thiazole-4-carboxylate (43.3)

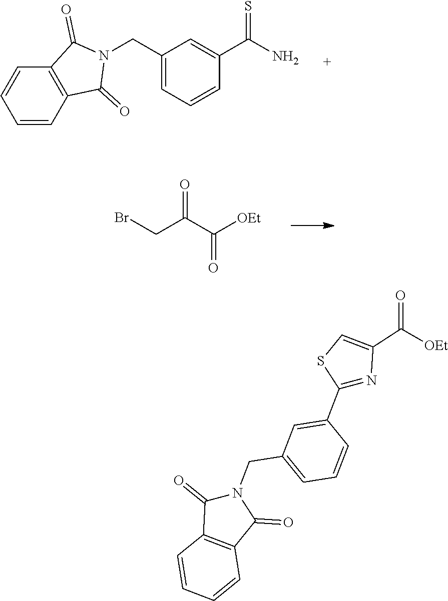 [SMILES: NC(=S)c1cccc(CN2C(=O)c3ccccc3C2=O)c1 ; CCOC(=O)C(=O)CBr ; CCOC(=O)c1csc(-c2cccc(CN3C(=O)c4ccccc4C3=O)c2)n1]

3-((1,3-Dioxoisoindolin-2-yl)methyl)benzothioamide (3.145 g, 10.6 mmole) was dissolved in ethanol (21 mL). Ethyl bromopyruvate (1.60 mL, 12.8 mmole) was added and the mixture was heated to 80° C. for 3 hours, then was cooled to RT. The precipitated solid was collected by filtration to provide ethyl 2-(3-((1,3-dioxoisoindolin-2-yl)methyl)phenyl)thiazole-4-carboxylate as a white solid (2.928 g, 78% from TLS-023-10-1).

¹H NMR (400 MHz, d₆-DMSO): δ 8.57 (s, 1H), 8.01-7.95 (m, 1H), 7.95-7.82 (m, 5H), 7.55-7.43 (m, 2H), 4.88 (s, 2H), 4.34 (q, J=7.1 Hz, 2H), 1.33 (t, J=7.1 Hz, 3H).

### Ethyl 2-(3-(((tert-butoxycarbonyl)amino)methyl)phenyl)thiazole-4-carboxylate (43.4)

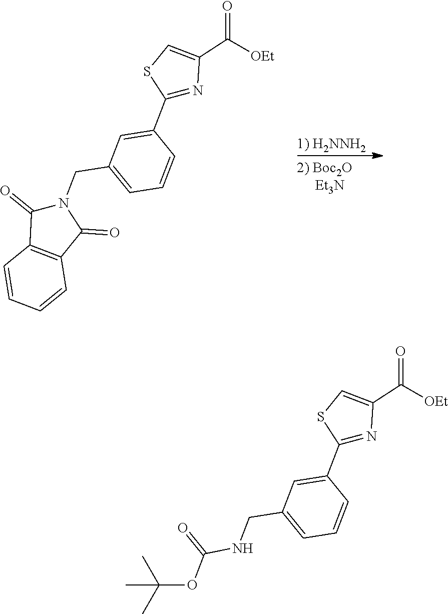 [SMILES: CCOC(=O)c1csc(-c2cccc(CN3C(=O)c4ccccc4C3=O)c2)n1 ; CCOC(=O)c1csc(-c2cccc(CNC(=O)OC(C)(C)C)c2)n1]

Ethyl 2-(3-((1,3-dioxoisoindolin-2-yl)methyl)phenyl)thiazole-4-carboxylate (2.928 g, 7.46 mmole) was suspended in ethanol (15 mL). Hydrazine (1M solution in THF, 15.0 mL, 15.0 mmole) was added and the mixture was heated to 80° C. for 2 hours, then was cooled to RT. The precipitated solid was filtered and washed with ethanol and the combined filtrates were concentrated. The residue was dissolved in DCM (15 mL) and triethylamine (1.25 mL, 8.92 mmole) and di-tert-butyl dicarbonate (1.788 g, 8.19 mmole) were added. The mixture was stirred at RT for 4 hours and water (50 mL) was added. The two layers were separated and the aqueous layer was extracted with DCM (2×10 mL) and the combined organics were dried over magnesium sulfate, filtered, and concentrated. The crude residue was purified by silica gel chromatography to provide ethyl 2-(3-(((tert-butoxycarbonyl)amino)methyl)phenyl)thiazole-4-carboxylate as a white solid (1.900 g, 70%).

¹H NMR (400 MHz, CDCl₃): δ 8.15 (s, 1H), 7.94 (d, J=2.0 Hz, 1H), 7.87 (dt, J=7.0, 2.0 Hz, 1H), 7.45-7.35 (m, 2H), 4.95 (s, 1H), 4.45 (q, J=7.1 Hz, 2H), 4.38 (d, J=6.1 Hz, 2H), 1.47 (s, 9H), 1.43 (t, J=7.1 Hz, 3H).

### Methyl N-(2-(3-(((tert-butoxycarbonyl)amino)methyl)phenyl)thiazole-4-carbonyl)-O-(tert-butyldimethylsilyl)-L-serinate (43.5)

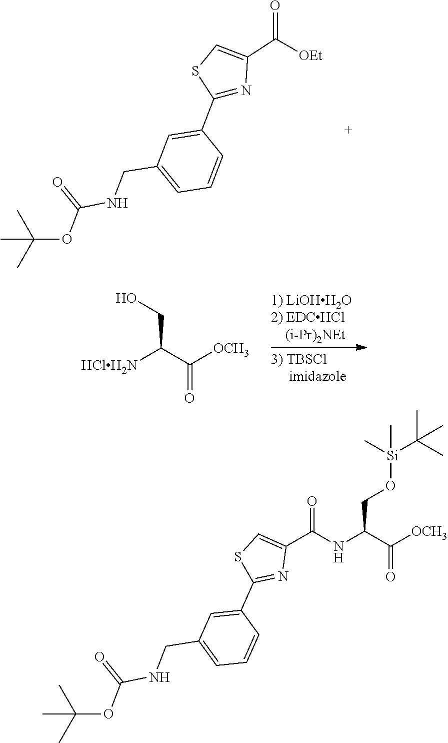

Ethyl 2-(3-(((tert-butoxycarbonyl)amino)methyl)phenyl)thiazole-4-carboxylate (0.500 g, 1.38 mmole) was dissolved in 4/1/1 THF/methanol/water (2.8 mL). Lithium hydroxide monohydrate (0.118 g, 2.81 mmole) was added and the mixture was stirred at RT for 2 hours. Water (25 mL) was added and the solution was treated with 1N aq. HCl to pH=4. The mixture was extracted with ethyl acetate (3×10 mL) and the combined organics were dried over magnesium sulfate, filtered, and concentrated. The residue was dissolved in DCM (5.6 mL) and L-serine methyl ester hydrochloride (0.260 g, 1.67 mmole) was added, followed by N,N-diisopropylethylamine (0.480 mL, 2.76 mmole) and EDC·HCl (0.325 g, 1.70 mmole). The mixture was stirred at RT for 3 hours and water (25 mL) was added. The mixture was extracted with DCM (3×10 mL) and the combined organics were dried over magnesium sulfate, filtered, and concentrated. The residue was dissolved in DCM (5.6 mL) and imidazole (0.108 g, 1.59 mmole) and tert-butyldimethylchlorosilane (0.230 g, 1.53 mmole) were added. The mixture was stirred at RT for 3 hours and water (25 mL) was added. The mixture was extracted with DCM (3×10 mL) and the combined organics were dried over magnesium sulfate, filtered, and concentrated. The crude residue was purified by silica gel chromatography to provide methyl N-(2-(3-(((tert-butoxycarbonyl)amino)methyl)phenyl)thiazole-4-carbonyl)-O-(tert-butyldimethylsilyl)-L-serinate as a colorless oil (0.522 g, 69%).

¹H NMR (400 MHz, CDCl₃): δ 8.19 (d, J=8.8 Hz, 1H), 8.10 (s, 1H), 7.88 (dt, J=6.7, 2.0 Hz, 1H), 7.83 (dq, J=1.8, 0.9 Hz, 1H), 7.45-7.38 (m, 2H), 4.94 (s, 1H), 4.89-4.81 (m, 1H), 4.38 (d, J=6.0 Hz, 2H), 4.20 (dd, J=10.0, 2.7 Hz, 1H), 3.95 (dd, J=10.1, 3.4 Hz, 1H), 3.78 (s, 3H), 1.47 (s, 9H), 0.91 (s, 9H), 0.07 (s, 3H), 0.05 (s, 3H).

### Methyl O-acetyl-N—(N-(2-(3-(((tert-butoxycarbonyl)amino)methyl)phenyl)thiazole-4-carbonyl)-O-(tert-butyldimethylsilyl)-L-seryl)-L-serinate (43.6)

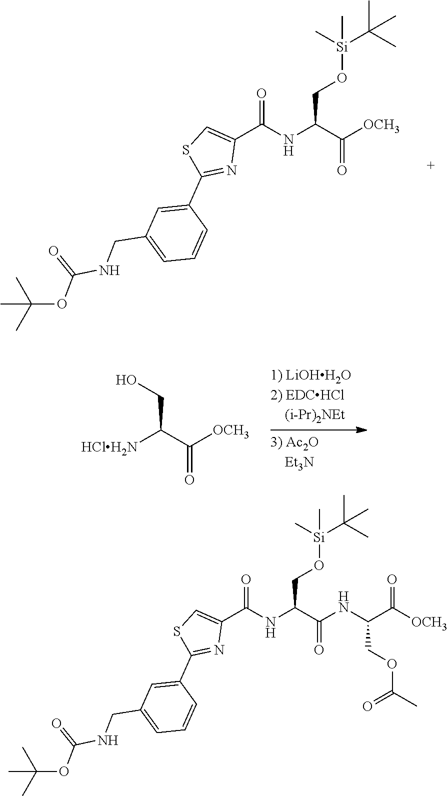

Methyl N-(2-(3-(((tert-butoxycarbonyl)amino)methyl)phenyl)thiazole-4-carbonyl)-O-(tert-butyldimethylsilyl)-L-serinate (0.522 g, 0.950 mmole) was dissolved in 4/1/1 THF/methanol/water (3 mL). Lithium hydroxide monohydrate (0.063 g, 1.50 mmole) was added and the mixture was stirred at RT for 2 hours. Water (25 mL) was added and the solution was treated with 1N aq. HCl to pH=4. The mixture was extracted with ethyl acetate (3×10 mL) and the combined organics were dried over magnesium sulfate, filtered, and concentrated. The residue was dissolved in DCM (3 mL). L-Serine methyl ester hydrochloride (0.177 g, 1.14 mmole) was added followed by N,N-diisopropylethylamine (0.330 mL, 1.89 mmole) and EDC·HCl (0.218 g, 1.14 mmole). The mixture was stirred at RT for 18 hours and water (25 mL) was added. The mixture was extracted with DCM (3×10 mL) and the combined organics were dried over magnesium sulfate, filtered and concentrated. The residue was dissolved in DCM (3 mL). Triethylamine (0.150 mL, 1.07 mmole) and acetic anhydride (0.100 mL, 1.06 mmole) were added and the mixture was stirred at RT for 3 hours. Water (25 mL) was added and the mixture was extracted with DCM (3×10 mL). The combined organics were dried over magnesium sulfate, filtered and concentrated. The crude residue was purified by silica gel chromatography to provide methyl O-acetyl-N—(N-(2-(3-(((tert-butoxycarbonyl)amino)methyl)phenyl)thiazole-4-carbonyl)-O-(tert-butyldimethylsilyl)-L-seryl)-L-serinate as a white solid (0.266 g, 41%).

¹H NMR (400 MHz, CDCl₃): δ 8.23 (dd, J=7.2, 2.6 Hz, 1H), 8.12 (d, J=2.1 Hz, 1H), 7.89 (dt, J=6.4, 2.1 Hz, 1H), 7.86-7.81 (m, 1H), 7.50-7.44 (m, 1H), 7.41 (td, J=6.7, 5.4, 3.2 Hz, 2H), 4.95 (s, 1H), 4.90 (dq, J=6.9, 3.5 Hz, 1H), 4.67 (td, J=7.3, 3.8 Hz, 1H), 4.53-4.44 (m, 1H), 4.43-4.37 (m, 2H), 4.33 (dd, J=11.4, 3.6 Hz, 1H), 4.21 (ddd, J=9.9, 3.8, 2.4 Hz, 1H), 3.84-3.79 (m, 1H), 3.77 (s, 3H), 2.01 (s, 3H), 1.47 (s, 9H), 0.95 (s, 9H), 0.16 (s, 3H), 0.15 (s, 3H).

### Methyl 2-(2-(2-(3-(((tert-butoxycarbonyl)amino)methyl)phenyl)thiazole-4-carboxamido)acrylamido)acrylate (43)

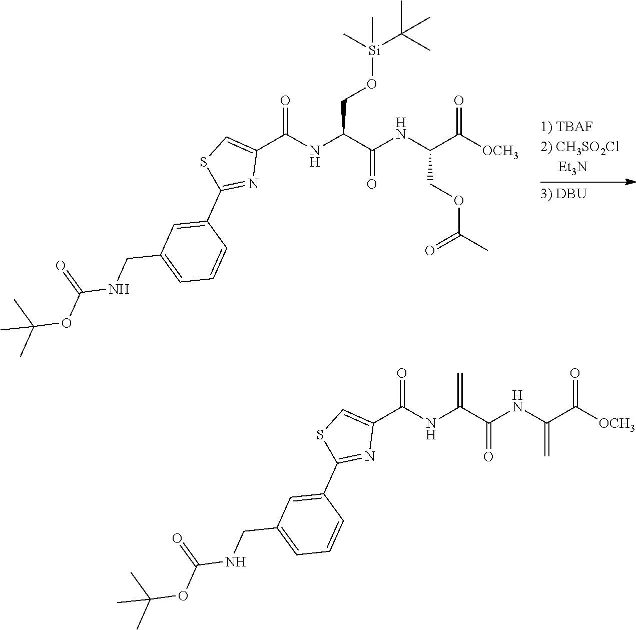 [SMILES: COC(=O)[C@H](COC(C)=O)NC(=O)[C@H](CO[Si](C)(C)C(C)(C)C)NC(=O)c1csc(-c2cccc(CNC(=O)OC(C)(C)C)c2)n1 ; C=C(NC(=O)c1csc(-c2cccc(CNC(=O)OC(C)(C)C)c2)n1)C(=O)NC(=C)C(=O)OC]

Methyl O-acetyl-N—(N-(2-(3-(((tert-butoxycarbonyl)amino)methyl)phenyl)thiazole-4-carbonyl)-O-(tert-butyldimethylsilyl)-L-seryl)-L-serinate (0.266 g, 0.392 mmole) was dissolved in THF (2 mL). TBAF (1M solution in THF, 0.980 mL, 0.980 mmole) was added and the solution was stirred at RT for 3 hours. Water (25 mL) was added and the mixture was extracted with ethyl acetate (3×10 mL). The combined organics were dried over magnesium sulfate, filtered and concentrated. The residue was dissolved in DCM (2 mL) and cooled to 0° C. Triethylamine (0.083 mL, 0.592 mmole) and methanesulfonyl chloride (0.046 mL, 0.594 mmole) were added and the mixture was stirred at 0° for 60 minutes. Water (25 mL) was added and the mixture was extracted with DCM (3×10 mL). The combined organics were dried over magnesium sulfate, filtered and concentrated. The residue was dissolved in THF (2 mL) and cooled to 0° C. DBU (0.090 mL, 0.602 mmole) was added and the mixture was stirred at 0° for 60 minutes. Water (25 mL) was added and the mixture was extracted with ethyl acetate (3×10 mL). The combined organics were dried over magnesium sulfate, filtered and concentrated. The crude residue was purified by silica gel chromatography to provide methyl 2-(2-(2-(3-(((tert-butoxycarbonyl)amino)methyl)phenyl)thiazole-4-carboxamido)acrylamido)acrylate as a white solid (0.088 g, 46%).

¹H NMR (400 MHz, CDCl₃): δ 9.98 (s, 1H), 8.55 (s, 1H), 8.16 (s, 1H), 7.94 (dt, J=7.3, 1.7 Hz, 1H), 7.86 (td, J=1.8, 0.8 Hz, 1H), 7.48-7.38 (m, 2H), 6.78 (d, J=2.2 Hz, 1H), 6.72 (s, 1H), 6.03 (d, J=1.3 Hz, 1H), 5.51 (t, J=1.9 Hz, 1H), 4.97 (s, 1H), 4.41 (d, J=6.1 Hz, 2H), 3.90 (s, 3H), 1.48 (s, 9H).

### Compound 44: Methyl 2-(2-(2-(4-((6-((tert-butoxycarbonyl)amino)hexanamido)methyl)phenyl)thiazole-4-carboxamido)acrylamido)acrylate

### Methyl N-(2-(4-((6-((tert-butoxycarbonyl)amino)hexanamido)methyl)phenyl)thiazole-4-carbonyl)-O-(tert-butyldimethylsilyl)-L-serinate (44.1)

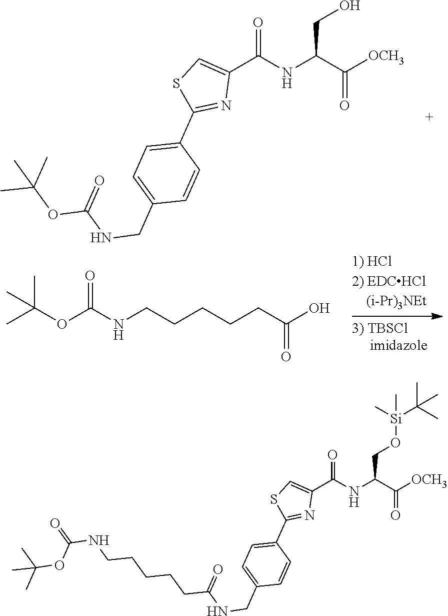 [SMILES: COC(=O)[C@H](CO)NC(=O)c1csc(-c2ccc(CNC(=O)OC(C)(C)C)cc2)n1 ; CC(C)(C)OC(=O)NCCCCCC(=O)O ; COC(=O)[C@H](CO[Si](C)(C)C(C)(C)C)NC(=O)c1csc(-c2ccc(CNC(=O)CCCCCNC(=O)OC(C)(C)C)cc2)n1]

Methyl (2-(4-(((tert-butoxycarbonyl)amino)methyl)phenyl)thiazole-4-carbonyl)-L-serinate (Example 35.4, 1.440 g, 3.31 mmole) was dissolved in DCM (6.5 mL). HCl (4M solution in 1,4-dioxane, 3.30 mL, 13.2 mmole) was added and the mixture was stirred at RT for 2 hours, then was concentrated. The residue was suspended in DCM (6.5 mL) and 6-((tert-butoxycarbonyl)amino)hexanoic acid (0.993 g, 4.29 mmole) was added. N,N-Diisopropylethylamine (1.15 mL, 6.60 mmole) was added followed by EDC·HCl (0.822 g, 4.29 mmole) and the mixture was stirred at RT for 18 hours. Water (25 mL) was added and the mixture was extracted with DCM (3×10 mL). The combined organics were dried over magnesium sulfate, filtered and concentrated. The residue was dissolved in DCM (13 mL) and imidazole (0.249 g, 3.66 mmole) and tert-butyldimethylchlorosilane (0.560 g, 3.72 mmole) were added. The mixture was stirred at RT for 2 hours before water (25 mL) was added. The mixture was extracted with DCM (3×10 mL) and the combined organics were dried over magnesium sulfate, filtered and concentrated. The crude residue was purified by silica gel chromatography to provide methyl N-(2-(4-((6-((tert-butoxycarbonyl)amino)hexanamido)methyl)phenyl)thiazole-4-carbonyl)-O-(tert-butyldimethylsilyl)-L-serinate as a colorless oil (1.781 g, 81%).

¹H NMR (400 MHz, CDCl₃): δ 8.21 (d, J=8.7 Hz, 1H), 8.07 (s, 1H), 7.95-7.88 (m, 2H), 7.39-7.31 (m, 2H), 6.01 (s, 1H), 4.89-4.81 (m, 1H), 4.57 (s, 1H), 4.49 (d, J=5.9 Hz, 2H), 4.21 (dd, J=10.1, 2.7 Hz, 1H), 3.95 (dd, J=10.1, 3.4 Hz, 1H), 3.79 (s, 3H), 3.11 (q, J=6.7 Hz, 2H), 2.26 (t, J=7.5 Hz, 2H), 1.75-1.66 (m, 3H), 1.57-1.47 (m, 2H), 1.43 (s, 9H), 1.40-1.32 (m, 2H), 0.92 (s, 9H), 0.08 (s, 3H), 0.06 (s, 3H).

### Methyl O-acetyl-N—(N-(2-(4-((6-((tert-butoxycarbonyl)amino)hexanamido)methyl)phenyl)thiazole-4-carbonyl)-O-(tert-butyldimethylsilyl)-L-seryl)-L-serinate (44.2)

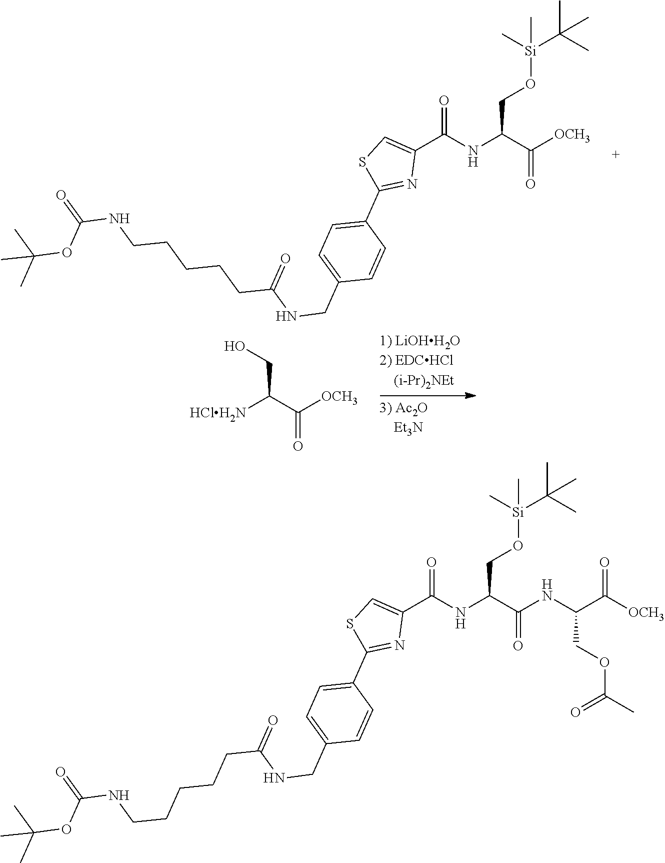

Methyl N-(2-(4-((6-((tert-butoxycarbonyl)amino)hexanamido)methyl)phenyl)thiazole-4-carbonyl)-O-(tert-butyldimethylsilyl)-L-serinate (1.781 g, 2.69 mmole) was dissolved in 4/1/1 THF/methanol/water (5.4 mL). Lithium hydroxide monohydrate (0.233 g, 5.55 mmole) was added and the mixture was stirred at RT for 3 hours and water (25 mL) was added. The mixture was treated with 1N aq. HCl to pH=4 and extracted with ethyl acetate (3×10 mL). The combined organics were dried over magnesium sulfate, filtered and concentrated. The residue was dissolved in DCM (5.4 mL) and L-serine methyl ester hydrochloride (0.538 g, 3.46 mmole) was added. N,N-Diisopropylethylamine (0.940 mL, 5.40 mmole) and EDC·HCl (0.677 g, 3.53 mmole) were added and the mixture was stirred at RT for 20 hours. Water (25 mL) was added and the two layers were separated. The aqueous layer was extracted with DCM (2×10 mL) and the combined organics were dried over magnesium sulfate, filtered and concentrated. The residue was dissolved in DCM (5.4 mL) and triethylamine (0.420 mL, 3.00 mmole) and acetic anhydride (0.280 mL, 2.96 mmole) were added. The mixture was stirred at RT for 20 hours and water (25 mL) was added. The two layers were separated and the aqueous layer was extracted with DCM (2×10 mL). The combined organics were dried over magnesium sulfate, filtered and concentrated. The crude residue was purified by silica gel chromatography to provide methyl O-acetyl-N—(N-(2-(4-((6-((tert-butoxycarbonyl)amino)hexanamido)methyl)phenyl)thiazole-4-carbonyl)-O-(tert-butyldimethylsilyl)-L-seryl)-L-serinate as a colorless oil (1.092 g, 51%).

¹H NMR (400 MHz, CDCl₃): δ 8.26 (d, J=7.1 Hz, 1H), 8.10 (d, J=2.7 Hz, 1H), 7.97-7.89 (m, 2H), 7.48 (d, J=7.7 Hz, 1H), 7.37-7.31 (m, 2H), 6.11-5.90 (m, 1H), 4.90 (dq, J=6.9, 3.5 Hz, 1H), 4.66 (td, J=7.3, 3.7 Hz, 1H), 4.58 (d, J=7.5 Hz, 1H), 4.53-4.47 (m, 3H), 4.44 (td, J=6.2, 4.1 Hz, 1H), 4.34 (dd, J=11.4, 3.6 Hz, 1H), 4.22 (dt, J=9.9, 3.5 Hz, 1H), 3.85-3.80 (m, 1H), 3.79-3.76 (m, 3H), 3.11 (q, J=6.7 Hz, 2H), 2.26 (t, J=7.5 Hz, 3H), 2.02 (d, J=6.8 Hz, 3H), 1.71 (p, J=7.5 Hz, 2H), 1.51 (p, J=7.3 Hz, 2H), 1.43 (s, 9H), 1.40-1.32 (m, 2H), 0.96 (s, 9H), 0.17 (s, 3H), 0.15 (s, 3H).

### Methyl 2-(2-(2-(4-((6-((tert-butoxycarbonyl)amino)hexanamido)methyl)phenyl)thiazole-4-carboxamido)acrylamido)acrylate (44)

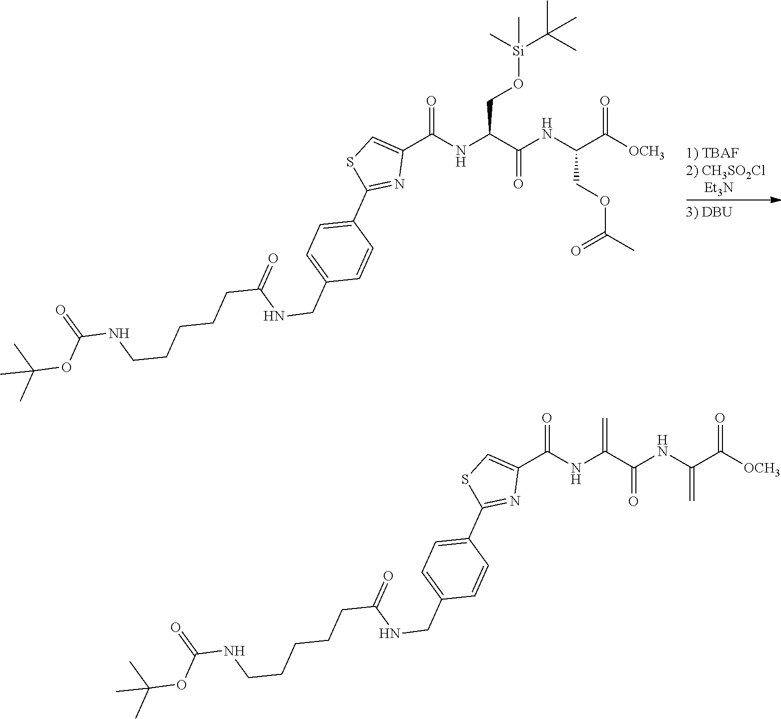 [SMILES: COC(=O)[C@H](COC(C)=O)NC(=O)[C@H](CO[Si](C)(C)C(C)(C)C)NC(=O)c1csc(-c2ccc(CNC(=O)CCCCCNC(=O)OC(C)(C)C)cc2)n1 ; C=C(NC(=O)c1csc(-c2ccc(CNC(=O)CCCCCNC(=O)OC(C)(C)C)cc2)n1)C(=O)NC(=C)C(=O)OC]

Methyl O-acetyl-N—(N-(2-(4-((6-((tert-butoxycarbonyl)amino)hexanamido)methyl)phenyl)thiazole-4-carbonyl)-O-(tert-butyldimethylsilyl)-L-seryl)-L-serinate (1.092 g, 1.38 mmole) was dissolved in THF (2.8 mL). TBAF (1M solution in THF, 3.50 mL, 3.50 mmole) was added and the solution was stirred at RT for 2 hours. Water (25 mL) was added and the mixture was extracted with ethyl acetate (3×10 mL). The combined organics were dried over magnesium sulfate, filtered and concentrated. The residue was dissolved in DCM (5.6 mL) and cooled to 0° C. Triethylamine (0.290 mL, 2.07 mmole) and methanesulfonyl chloride (0.160 mL, 2.07 mmole) were added and the mixture was stirred at 0° for 60 minutes. Water (25 mL) was added and the mixture was extracted with DCM (3×10 mL). The combined organics were dried over magnesium sulfate, filtered and concentrated. The residue was dissolved in THF (5.6 mL) and cooled to 0° C. DBU (0.310 mL, 2.07 mmole) was added and the mixture was stirred at 0° for 60 minutes. Water (25 mL) was added and the mixture was extracted with ethyl acetate (3×10 mL). The combined organics were dried over magnesium sulfate, filtered and concentrated. The crude residue was purified by silica gel chromatography to provide methyl 2-(2-(2-(4-((6-((tert-butoxycarbonyl)amino)hexanamido)methyl)phenyl)thiazole-4-carboxamido)acrylamido)acrylate as a white solid (0.255 g, 31%).

¹H NMR (400 MHz, CDCl₃): δ 10.01 (s, 1H), 8.55 (s, 1H), 8.14 (s, 1H), 8.03-7.91 (m, 2H), 7.37 (d, J=8.2 Hz, 2H), 6.78 (d, J=2.2 Hz, 1H), 6.71 (s, 1H), 6.09-6.00 (m, 1H), 5.86 (s, 1H), 5.50 (t, J=1.9 Hz, 1H), 4.55 (s, 1H), 4.50 (d, J=5.8 Hz, 2H), 3.91 (s, 3H), 3.12 (q, J=6.7 Hz, 2H), 2.26 (t, J=7.5 Hz, 2H), 1.71 (p, J=7.6 Hz, 2H), 1.51 (q, J=7.3 Hz, 2H), 1.43 (s, 9H), 1.40-1.32 (m, 2H).

### Compound 45: Methyl 2-(2-(2-(4-(((((tetrahydro-2H-pyran-4-yl)methoxy)carbonyl)amino)methyl)phenyl)thiazole-4-carboxamido)acrylamido)acrylate

### Ethyl 2-(4-(aminomethyl)phenyl)thiazole-4-carboxylate hydrobromide (45.1)

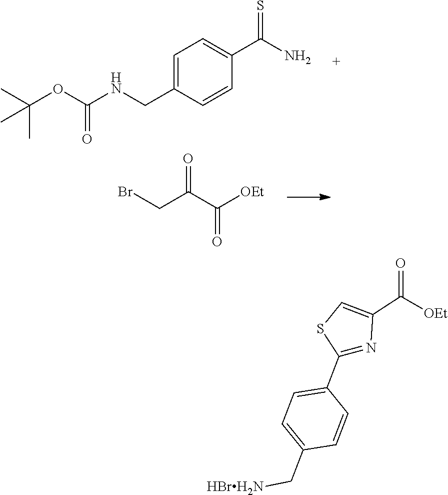

Tert-butyl (4-carbamothioylbenzyl)carbamate (Example 35.2, 7.744 g, 29.1 mmole) was dissolved in ethanol (60 mL) and ethyl bromopyruvate (4.40 mL, 35.1 mmole) was added. The mixture was stirred at 80° C. for 3 hours and was cooled to RT. The precipitated solid was collected by filtration, washed with a small amount of ethanol and allowed to air dry to provide ethyl 2-(4-(aminomethyl)phenyl)thiazole-4-carboxylate hydrobromide as a pale yellow solid (7.650 g, 77%).

¹H NMR (400 MHz, d₆-DMSO): δ 8.61 (s, 1H), 8.44-8.24 (m, 3H), 8.11-8.01 (m, 2H), 7.69-7.61 (m, 2H), 4.35 (q, J=7.1 Hz, 2H), 4.17-4.09 (m, 2H), 1.34 (t, J=7.1 Hz, 3H).

### Ethyl 2-(4-(((((tetrahydro-2H-pyran-4-yl)methoxy)carbonyl)amino)methyl)phenyl)thiazole-4-carboxylate (45.2)

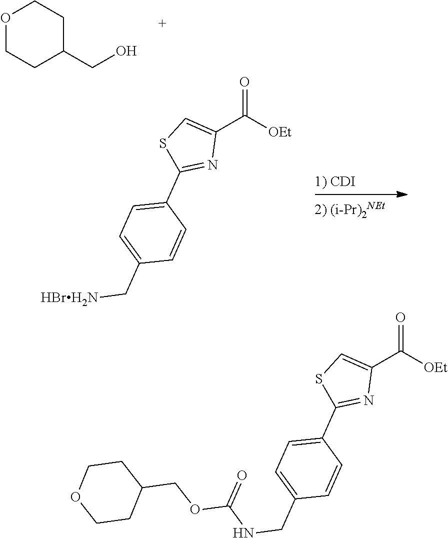

(Tetrahydro-2H-pyran-4-yl)methanol (0.279 g, 2.40 mmole) was dissolved in THF (4.2 mL) and CDI (0.408 g, 2.52 mmole) was added slowly. The mixture was stirred at RT for 18 hours. Water (25 mL) was added and the mixture was extracted with ethyl acetate (3×10 mL). The combined organics were dried over magnesium sulfate, filtered and concentrated. The residue was dissolved in DMF (4.2 mL) and ethyl 2-(4-(aminomethyl)phenyl)thiazole-4-carboxylate hydrobromide (0.753 g, 2.19 mmole) and N,N-diisopropylethylamine (0.460 mL, 2.64 mmole) were added. The mixture was warmed to 50° C. for 18 hours, then was cooled to RT and diluted with ethyl acetate (25 mL). The mixture was washed sequentially with water (3×25 mL) and sat. aq. sodium chloride (1×25 mL), then dried over magnesium sulfate, filtered and concentrated. The crude residue was purified by silica gel chromatography to provide ethyl 2-(4-(((((tetrahydro-2H-pyran-4-yl)methoxy)carbonyl)amino)methyl)phenyl)thiazole-4-carboxylate as an orange solid (0.613 g, 69%).

¹H NMR (400 MHz, CDCl₃): δ 8.15 (s, 1H), 8.03-7.94 (m, 2H), 7.37 (d, J=7.9 Hz, 2H), 5.09 (s, 1H), 4.50-4.35 (m, 4H), 3.97 (t, J=5.5 Hz, 4H), 3.39 (t, J=11.1 Hz, 2H), 1.61 (s, 2H), 1.43 (t, J=7.1 Hz, 3H), 1.40-1.29 (m, 2H).

### Methyl O-(tert-butyldimethylsilyl)-N-(2-(4-(((((tetrahydro-2H-pyran-4-yl)methoxy)carbonyl)amino)methyl)phenyl)thiazole-4-carbonyl)-L-serinate (45.3)

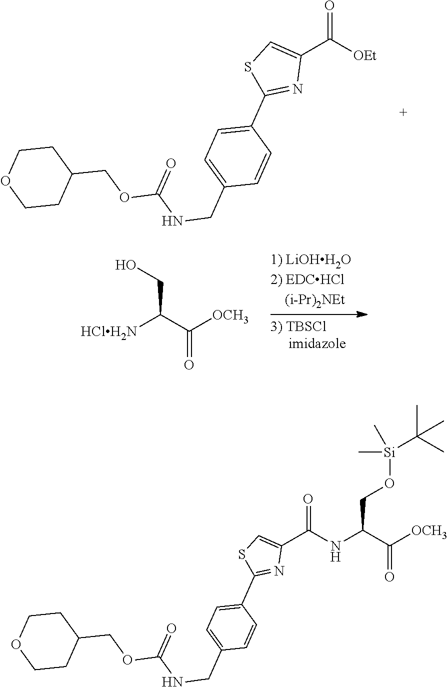

Ethyl 2-(4-(((((tetrahydro-2H-pyran-4-yl)methoxy)carbonyl)amino)methyl)phenyl)thiazole-4-carboxylate (0.613 g, 1.52 mmole) was dissolved in 4/1/1 THF/methanol/water (3 mL). Lithium hydroxide monohydrate (0.097 g, 2.31 mmole) was added and the mixture was stirred at RT for 3 hours. Water (25 mL) was added and the solution was treated with 1N aq. HCl to pH=4. The mixture was extracted with ethyl acetate (3×10 mL). The combined organics were dried over magnesium sulfate, filtered and concentrated. The residue was suspended in DCM (6 mL). L-Serine methyl ester hydrochloride (0.307 g, 1.97 mmole) was added followed by N,N-diisopropylethylamine (0.530 mL, 3.04 mmole) and EDC·HCl (0.385 g, 2.01 mmole). The mixture was stirred at RT for 2 days. Water (25 mL) was added and the mixture was extracted with DCM (3×10 mL). The combined organics were dried over magnesium sulfate, filtered and concentrated. The residue was dissolved in DCM (3 mL) and imidazole (0.120 g, 1.76 mmole) and tert-butyldimethylchlorosilane (0.250 g, 1.66 mmole) were added. The mixture was stirred at RT for 3 hours and water (25 mL) was added. The mixture was extracted with DCM (3×10 mL) and the combined organics were dried over magnesium sulfate, filtered and concentrated. The crude residue was purified by silica gel chromatography to provide methyl O-(tert-butyldimethylsilyl)-N-(2-(4-(((((tetrahydro-2H-pyran-4-yl)methoxy)carbonyl)amino)methyl)phenyl)thiazole-4-carbonyl)-L-serinate as a thick, colorless oil (0.329 g, 37%).

¹H NMR (400 MHz, CDCl₃): δ 8.21 (d, J=8.7 Hz, 1H), 8.10 (s, 1H), 7.99-7.88 (m, 2H), 7.38 (d, J=7.8 Hz, 2H), 5.09 (s, 1H), 4.86 (dt, J=8.7, 3.0 Hz, 1H), 4.43 (d, J=6.1 Hz, 2H), 4.21 (dd, J=10.0, 2.6 Hz, 1H), 4.04-3.92 (m, 5H), 3.79 (s, 3H), 3.39 (t, J=11.8 Hz, 2H), 1.90 (q, J=7.9, 5.7 Hz, 1H), 1.61 (s, 3H), 1.39 (tt, J=14.2, 6.9 Hz, 2H), 0.92 (s, 9H), 0.07 (s, 3H), 0.06 (s, 3H).

### Methyl O-acetyl-N—(O-(tert-butyldimethylsilyl)-N-(2-(4-(((((tetrahydro-2H-pyran-4-yl)methoxy)carbonyl)amino)methyl)phenyl)thiazole-4-carbonyl)-L-seryl)-L-serinate (45.4)

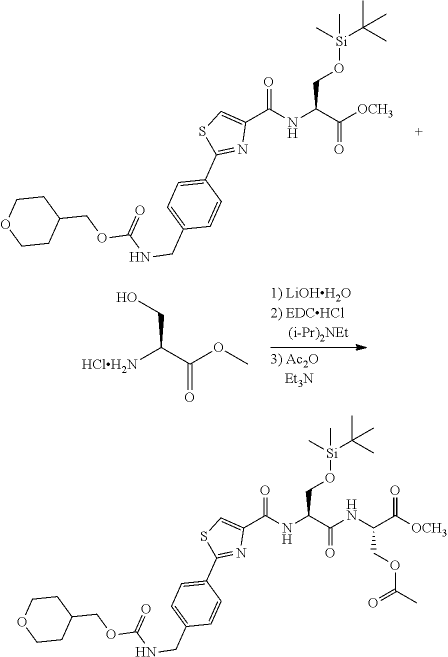

Methyl O-(tert-butyldimethylsilyl)-N-(2-(4-(((((tetrahydro-2H-pyran-4-yl)methoxy)carbonyl)amino)methyl)phenyl)thiazole-4-carbonyl)-L-serinate (0.329 g, 0.556 mmole) was dissolved in 4/1/1 THF/methanol/water (2.2 mL). Lithium hydroxide monohydrate (0.039 g, 0.929 mmole) was added and the mixture was stirred at RT for 3 hours. Water (25 mL) was added and the solution was treated with 1N aq. HCl to pH=4. The mixture was extracted with ethyl acetate (6×10 mL) and the combined organics were dried over magnesium sulfate, filtered and concentrated. The residue was suspended in DCM (2.2 mL). L-Serine methyl ester hydrochloride (0.113 g, 0.726 mmole) was added followed by N,N-diisopropylethylamine (0.195 mL, 1.12 mmole) and EDC·HCl (0.146 g, 0.762 mmole). The mixture was stirred at RT for 18 hours and water (25 mL) was added. The mixture was extracted with DCM (3×10 mL) and the combined organics were dried over magnesium sulfate, filtered and concentrated. The residue was dissolved in DCM (2.2 mL) and triethylamine (0.086 mL, 0.614 mmole) and acetic anhydride (0.058 mL, 0.614 mmole) were added. The solution was stirred at RT for 2 hours and water (25 mL) was added. The mixture was extracted with DCM (3×10 mL) and the combined organics were dried over magnesium sulfate, filtered and concentrated. The crude residue was purified by silica gel chromatography to provide methyl O-acetyl-N—(O-(tert-butyldimethylsilyl)-N-(2-(4-(((((tetrahydro-2H-pyran-4-yl)methoxy)carbonyl)amino)methyl)phenyl)thiazole-4-carbonyl)-L-seryl)-L-serinate as a thick oil (0.223 g, 56%).

¹H NMR (400 MHz, CDCl₃): δ 8.26 (d, J=7.0 Hz, 1H), 8.11 (d, J=2.3 Hz, 1H), 7.95 (dd, J=8.3, 1.8 Hz, 2H), 7.47 (d, J=7.8 Hz, 1H), 7.37 (d, J=8.0 Hz, 2H), 5.06 (s, 1H), 4.90 (tt, J=7.4, 3.8 Hz, 1H), 4.66 (td, J=7.3, 3.7 Hz, 1H), 4.54-4.45 (m, 1H), 4.45-4.38 (m, 2H), 4.34 (dd, J=11.4, 3.6 Hz, 1H), 4.23 (ddd, J=9.8, 3.8, 2.7 Hz, 1H), 3.99 (d, J=6.5 Hz, 4H), 3.82 (dd, J=10.6, 3.1 Hz, 1H), 3.77 (s, 3H), 3.39 (t, J=11.7 Hz, 2H), 2.01 (s, 2H), 1.98-1.83 (m, 1H), 1.63 (d, J=13.3 Hz, 2H), 1.39 (td, J=14.7, 13.4, 6.6 Hz, 2H), 0.96 (s, 9H), 0.17 (s, 3H), 0.15 (s, 3H).

### Methyl 2-(2-(2-(4-(((((tetrahydro-2H-pyran-4-yl)methoxy)carbonyl)amino)methyl)phenyl)thiazole-4-carboxamido)acrylamido)acrylate (45)

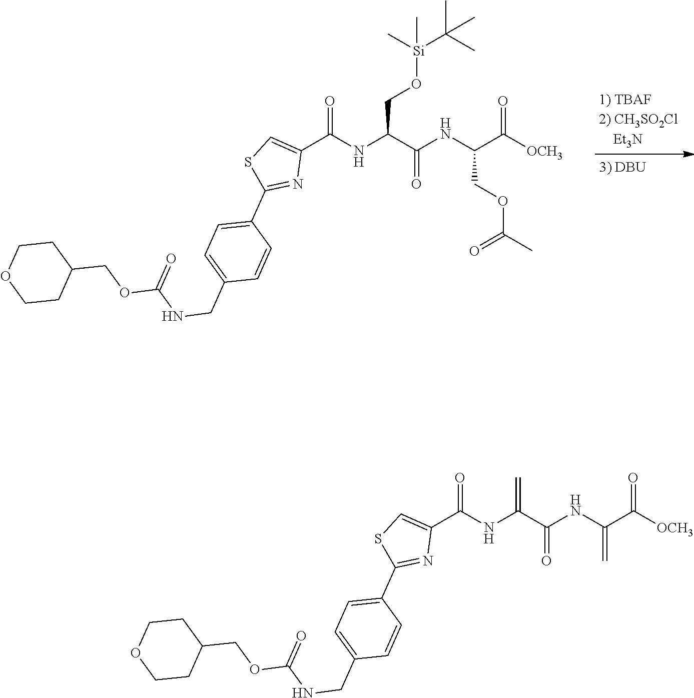 [SMILES: COC(=O)[C@H](COC(C)=O)NC(=O)[C@H](CO[Si](C)(C)C(C)(C)C)NC(=O)c1csc(-c2ccc(CNC(=O)OCC3CCOCC3)cc2)n1 ; C=C(NC(=O)c1csc(-c2ccc(CNC(=O)OCC3CCOCC3)cc2)n1)C(=O)NC(=C)C(=O)OC]

Methyl O-acetyl-N—(O-(tert-butyldimethylsilyl)-N-(2-(4-(((((tetrahydro-2H-pyran-4-yl)methoxy)carbonyl)amino)methyl)phenyl)thiazole-4-carbonyl)-L-seryl)-L-serinate (0.223 g, 0.309 mmole) was dissolved in THF (2 mL). TBAF (1M solution in THF, 0.780 mL, 0.780 mmole) was added and the solution was stirred at RT for 2 hours. Water (25 mL) and sat. aq. sodium chloride (10 mL) were added and the mixture was extracted with ethyl acetate (3×10 mL). The combined organics were dried over magnesium sulfate, filtered and concentrated. The residue was dissolved in DCM (2 mL) and cooled to 0° C. Triethylamine (0.065 mL, 0.464 mmole) and methanesulfonyl chloride (0.036 mL, 0.465 mmole) were added and the mixture was stirred at 0° for 60 minutes and water (25 mL) was added. The mixture was extracted with DCM (3×10 mL) and the combined organics were dried over magnesium sulfate, filtered and concentrated. The residue was dissolved in THF (2 mL) and cooled to 0° C. DBU (0.070 mL, 0.468 mmole) was added and the mixture was stirred at 0° for 45 minutes. Water (25 mL) was added and the mixture was extracted with DCM (3×10 mL). The combined organics were dried over magnesium sulfate, filtered and concentrated. The crude residue was purified by silica gel chromatography to provide methyl 2-(2-(2-(4-(((((tetrahydro-2H-pyran-4-yl)methoxy)carbonyl)amino)methyl)phenyl)thiazole-4-carboxamido)acrylamido)acrylate as a white solid (0.086 g, 53%).

¹H NMR (400 MHz, CDCl₃): δ 10.02 (s, 1H), 8.55 (s, 1H), 8.14 (s, 1H), 8.02-7.94 (m, 2H), 7.39 (d, J=7.9 Hz, 2H), 6.78 (d, J=2.2 Hz, 1H), 6.74-6.66 (m, 1H), 6.04 (d, J=1.2 Hz, 1H), 5.50 (t, J=1.9 Hz, 1H), 5.08 (s, 1H), 4.43 (d, J=6.1 Hz, 2H), 3.99 (d, J=6.5 Hz, 4H), 3.91 (s, 3H), 3.46-3.34 (m, 2H), 2.00-1.84 (m, 1H), 1.68-1.57 (m, 2H), 1.38 (qd, J=11.9, 4.0 Hz, 2H).

### Compound 46: Methyl 2-(2-(2-(4-(tetrahydro-2H-pyran-4-carboxamido)piperidin-1-yl)thiazole-4-carboxamido)acrylamido)acrylate

### Ethyl 2-(4-((tert-butoxycarbonyl)amino)piperidin-1-yl)thiazole-4-carboxylate (46.1)

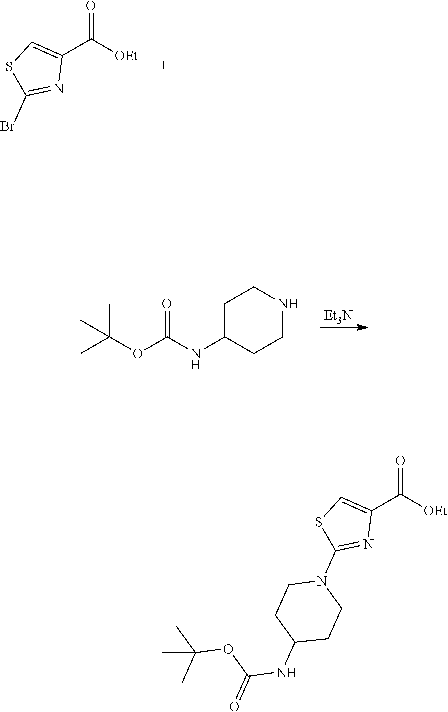 [SMILES: "Et3N" ; CCOC(=O)c1csc(Br)n1 ; CC(C)(C)OC(=O)NC1CCNCC1 ; CCOC(=O)c1csc(N2CCC(NC(=O)OC(C)(C)C)CC2)n1]

Ethyl 2-bromothiazole-4-carboxylate (3.021 g, 12.8 mmole) and tert-butyl piperidin-4-ylcarbamate (3.065 g, 15.3 mmole) were dissolved in DMA (13 mL). Triethylamine (2.15 mL, 15.3 mmole) was added and the mixture was heated to 80° C. for 18 hours, then was cooled to RT and diluted with ethyl acetate (50 mL). The mixture was washed with water (3×25 mL) and the organics were dried over magnesium sulfate, filtered, and concentrated. The crude residue was purified by silica gel chromatography to provide ethyl 2-(4-((tert-butoxycarbonyl)amino)piperidin-1-yl)thiazole-4-carboxylate as a white solid (4.073 g, 90%).

¹H NMR (400 MHz, CDCl₃): δ 7.42 (s, 1H), 4.48 (d, J=7.9 Hz, 1H), 4.34 (q, J=7.1 Hz, 2H), 4.03-3.94 (m, 2H), 3.67 (s, 1H), 3.14 (ddd, J=13.3, 11.6, 3.0 Hz, 2H), 2.09-1.98 (m, 2H), 1.55-1.46 (m, 2H), 1.44 (s, 9H), 1.36 (t, J=7.1 Hz, 3H).

### Methyl (2-(4-((tert-butoxycarbonyl)amino)piperidin-1-yl)thiazole-4-carbonyl)-L-serinate (46.2)

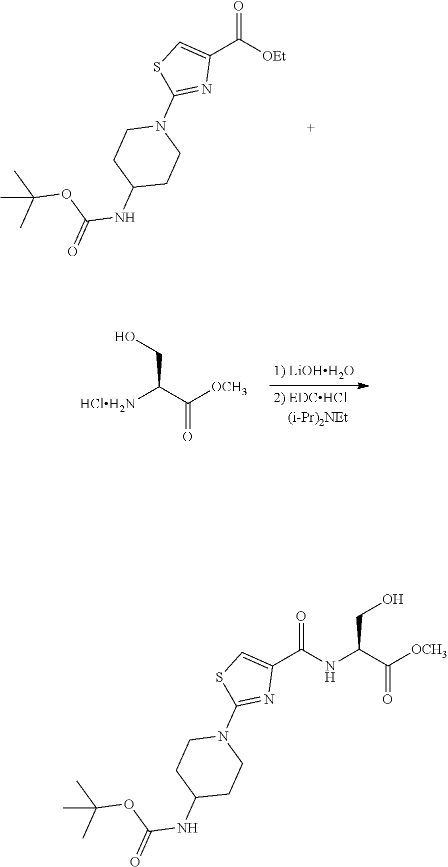

Ethyl 2-(4-((tert-butoxycarbonyl)amino)piperidin-1-yl)thiazole-4-carboxylate (3.371 g, 10.5 mmole) was dissolved in 4/1/1 THF/methanol/water (21 mL). Lithium hydroxide monohydrate (0.667 g, 15.9 mmole) was added and the mixture was stirred at RT for 2 hours. Water (50 mL) was added and the solution was treated with 1N aq. HCl to pH=4. The mixture was extracted with ethyl acetate (3×15 mL) and the combined organics were dried over magnesium sulfate, filtered and concentrated. The residue was suspended in DCM (21 mL) and L-serine methyl ester hydrochloride (2.119 g, 13.6 mmole) was added. N,N-Diisopropylethylamine (3.70 mL, 21.2 mmole) and EDC·HCl (2.629 g, 13.7 mmole) were added and the mixture was stirred at RT for 18 hours. Water (50 mL) was added and the two layers were separated. The aqueous layer was extracted with DCM (2×15 mL) and the combined organics were dried over magnesium sulfate, filtered and concentrated. The crude residue was purified by silica gel chromatography to provide methyl (2-(4-((tert-butoxycarbonyl)amino)piperidin-1-yl)thiazole-4-carbonyl)-L-serinate as a white solid (2.566 g, 57%).

¹H NMR (400 MHz, CDCl₃): δ 7.98 (d, J=7.4 Hz, 1H), 7.39 (s, 1H), 4.79 (dt, J=7.6, 3.9 Hz, 1H), 4.53 (s, 1H), 4.04 (ddd, J=6.2, 3.8, 1.0 Hz, 2H), 3.95 (dq, J=12.7, 3.6, 2.8 Hz, 2H), 3.82 (s, 3H), 3.66 (d, J=25.2 Hz, 1H), 3.21-3.08 (m, 2H), 2.87 (t, J=6.1 Hz, 1H), 2.11-1.99 (m, 2H), 1.56-1.48 (m, 2H), 1.46 (s, 9H).

### Methyl O-(tert-butyldimethylsilyl)-N-(2-(4-(tetrahydro-2H-pyran-4-carboxamido)piperidin-1-yl)thiazole-4-carbonyl)-L-serinate (46.3)

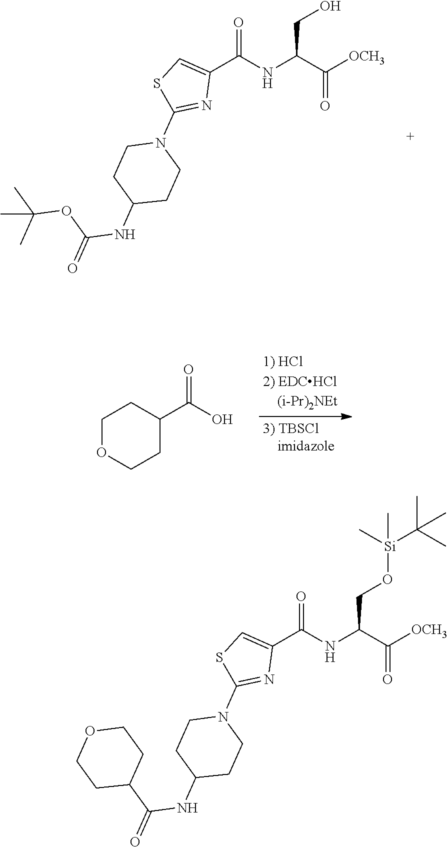 [SMILES: COC(=O)[C@H](CO)NC(=O)c1csc(N2CCC(NC(=O)OC(C)(C)C)CC2)n1 ; O=C(O)C1CCOCC1 ; COC(=O)[C@H](CO[Si](C)(C)C(C)(C)C)NC(=O)c1csc(N2CCC(NC(=O)C3CCOCC3)CC2)n1]

Methyl (2-(4-((tert-butoxycarbonyl)amino)piperidin-1-yl)thiazole-4-carbonyl)-L-serinate (0.510 g, 1.19 mmole) was dissolved in DCM (2.3 mL). HCl (4M solution in 1,4-dioxane, 1.20 mL, 4.80 mmole) was added and the mixture was stirred at RT for 4 hours, then was concentrated. The residue was suspended in DCM (2.3 mL) and tetrahydro-2H-pyran-4-carboxylic acid (0.195 g, 1.51 mmole) was added. N,N-Diisopropylethylamine (0.410 mL, 2.35 mmole) and EDC·HCl (0.294 g, 1.53 mmole) were added and the mixture was stirred at RT for 18 hours. Water (25 mL) was added and the mixture was extracted with DCM (3×10 mL). The combined organics were dried over magnesium sulfate, filtered and concentrated. The residue was dissolved in DCM (3 mL) and imidazole (0.090 g, 1.32 mmole) and tert-butyldimethylchlorosilane (0.195 g, 1.29 mmole) were added. The mixture was stirred at RT for 3 hours and water (25 mL) was added. The mixture was extracted with DCM (3×10 mL) and the combined organics were dried over magnesium sulfate, filtered and concentrated. The crude residue was purified by silica gel chromatography to provide methyl O-(tert-butyldimethylsilyl)-N-(2-(4-(tetrahydro-2H-pyran-4-carboxamido)piperidin-1-yl)thiazole-4-carbonyl)-L-serinate as a white solid (0.419 g, 63%).

¹H NMR (400 MHz, CDCl₃): δ 7.95 (d, J=8.8 Hz, 1H), 7.38 (s, 1H), 5.39 (d, J=7.9 Hz, 1H), 4.83-4.74 (m, 1H), 4.18-4.13 (m, 1H), 4.08-3.98 (m, 4H), 3.98-3.90 (m, 1H), 3.88 (dd, J=10.0, 3.4 Hz, 1H), 3.76 (s, 3H), 3.41 (td, J=11.4, 2.9 Hz, 2H), 3.22-3.08 (m, 2H), 2.32 (tt, J=11.0, 4.5 Hz, 1H), 2.04-1.98 (m, 2H), 1.87-1.70 (m, 4H), 1.50 (ddt, J=15.3, 12.0, 7.8 Hz, 2H), 0.88 (s, 9H), 0.05 (s, 3H), 0.03 (s, 3H).

### Methyl O-acetyl-N—(O-(tert-butyldimethylsilyl)-N-(2-(4-(tetrahydro-2H-pyran-4-carboxamido)piperidin-1-yl)thiazole-4-carbonyl)-L-seryl)-L-serinate (46.4)

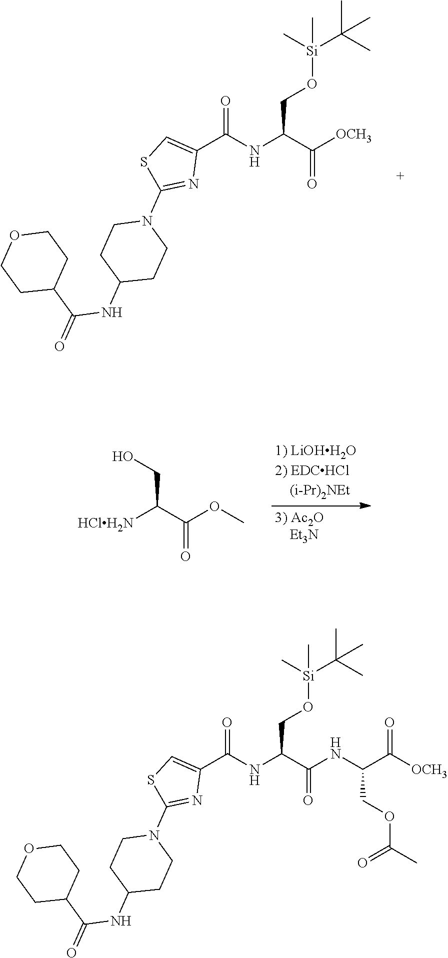

Methyl O-(tert-butyldimethylsilyl)-N-(2-(4-(tetrahydro-2H-pyran-4-carboxamido)piperidin 1-yl)thiazole-4-carbonyl)-L-serinate (0.419 g, 0.755 mmole) was dissolved in 4/1/1 THF/methanol/water (3.2 mL). Lithium hydroxide monohydrate (0.050 g, 1.19 mmole) was added and the mixture was stirred at RT for 3 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was suspended in DCM (3.2 mL). L-Serine methyl ester hydrochloride (0.152 g, 0.977 mmole) was added followed by N,N-diisopropylethylamine (0.265 mL, 1.52 mmole) and EDC·HCl (0.185 g, 0.965 mmole). The mixture was stirred at RT for 18 hours and water (25 mL) was added. The mixture was extracted with DCM (3×10 mL) and the combined organics were dried over magnesium sulfate, filtered and concentrated. The residue was dissolved in DCM (3.2 mL) and triethylamine (0.120 mL, 0.856 mmole) and acetic anhydride (0.080 mL, 0.846 mmole) were added. The solution was stirred at RT for 2 hours and water (25 mL) was added. The mixture was extracted with DCM (3×10 mL) and the combined organics were dried over magnesium sulfate, filtered and concentrated. The crude residue was purified by silica gel chromatography to provide methyl O-acetyl-N—(O-(tert-butyldimethylsilyl)-N-(2-(4-(tetrahydro-2H-pyran-4-carboxamido)piperidin-1-yl)thiazole-4-carbonyl)-L-seryl)-L-serinate as a colorless oil (0.212 g, 41%).

¹H NMR (400 MHz, CDCl₃): δ 7.98 (d, J=7.1 Hz, 1H), 7.44 (d, J=7.8 Hz, 1H), 7.40 (s, 1H), 5.38 (d, J=7.9 Hz, 1H), 4.87 (dt, J=7.6, 3.8 Hz, 1H), 4.58 (td, J=7.2, 3.7 Hz, 1H), 4.51-4.43 (m, 1H), 4.32 (dd, J=11.4, 3.7 Hz, 1H), 4.18 (dd, J=9.8, 3.7 Hz, 1H), 4.02 (ddd, J=11.5, 4.2, 2.5 Hz, 4H), 3.94 (dd, J=9.9, 6.4 Hz, 1H), 3.77 (s, 3H), 3.75-3.68 (m, 1H), 3.41 (td, J=11.4, 2.9 Hz, 2H), 3.21-3.05 (m, 2H), 2.32 (tt, J=11.0, 4.5 Hz, 1H), 2.02 (d, J=3.4 Hz, 4H), 1.84-1.71 (m, 4H), 1.50 (qq, J=11.6, 5.2 Hz, 2H), 0.92 (s, 9H), 0.14 (s, 3H), 0.13 (s, 3H).

### Methyl 2-(2-(2-(4-(tetrahydro-2H-pyran-4-carboxamido)piperidin-1-yl)thiazole-4-carboxamido)acrylamido)acrylate (46)

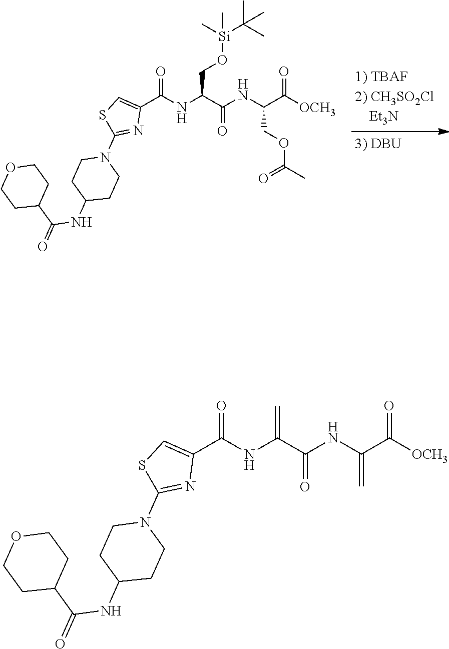 [SMILES: COC(=O)[C@H](COC(C)=O)NC(=O)[C@H](CO[Si](C)(C)C(C)(C)C)NC(=O)c1csc(N2CCC(NC(=O)C3CCOCC3)CC2)n1 ; C=C(NC(=O)c1csc(N2CCC(NC(=O)C3CCOCC3)CC2)n1)C(=O)NC(=C)C(=O)OC]

Methyl O-acetyl-N—(O-(tert-butyldimethylsilyl)-N-(2-(4-(tetrahydro-2H-pyran-4-carboxamido)piperidin-1-yl)thiazole-4-carbonyl)-L-seryl)-L-serinate (0.212 g, 0.310 mmole) was dissolved in THF (2 mL). TBAF (1M solution in THF, 0.780 mL, 0.780 mmole) was added and the solution was stirred at RT for 2 hours. Water (25 mL) and sat. aq. sodium chloride (10 mL) were added and the mixture was extracted with ethyl acetate (3×10 mL). The combined organics were dried over magnesium sulfate, filtered and concentrated. The residue was dissolved in DCM (2 mL) and cooled to 0° C. Triethylamine (0.066 mL, 0.471 mmole) and methanesulfonyl chloride (0.036 mL, 0.465 mmole) were added and the mixture was stirred at 0° for 60 minutes and water (25 mL) was added. The mixture was extracted with DCM (3×10 mL) and the combined organics were dried over magnesium sulfate, filtered and concentrated. The residue was dissolved in THF (2 mL) and cooled to 0° C. DBU (0.070 mL, 0.468 mmole) was added and the mixture was stirred at 0° for 45 minutes. Water (25 mL) was added and the mixture was extracted with DCM (3×10 mL). The combined organics were dried over magnesium sulfate, filtered and concentrated. The crude residue was purified by silica gel chromatography to provide methyl 2-(2-(2-(4-(tetrahydro-2H-pyran-4-carboxamido)piperidin-1-yl)thiazole-4-carboxamido)acrylamido)acrylate as a white solid (0.043 g, 28%).

¹H NMR (400 MHz, CDCl₃): δ 9.71 (s, 1H), 8.51 (s, 1H), 7.43 (s, 1H), 6.72 (d, J=2.1 Hz, 1H), 6.65 (s, 1H), 6.01 (d, J=1.3 Hz, 1H), 5.44 (t, J=1.9 Hz, 1H), 5.37 (d, J=7.8 Hz, 1H), 4.08-3.96 (m, 5H), 3.89 (s, 3H), 3.41 (td, J=11.4, 2.9 Hz, 2H), 3.17 (ddd, J=13.3, 11.7, 2.9 Hz, 2H), 2.32 (tt, J=11.0, 4.5 Hz, 1H), 2.10-2.02 (m, 2H), 1.88-1.70 (m, 4H), 1.57-1.45 (m, 2H).

### Compound 47: Methyl 2-(2-(2-(4-(2-(2-methoxyethoxy)acetyl)piperazin-1-yl)thiazole-4-carboxamido)acrylamido)acrylate

### Ethyl 2-(4-(tert-butoxycarbonyl)piperazin-1-yl)thiazole-4-carboxylate (47.1)

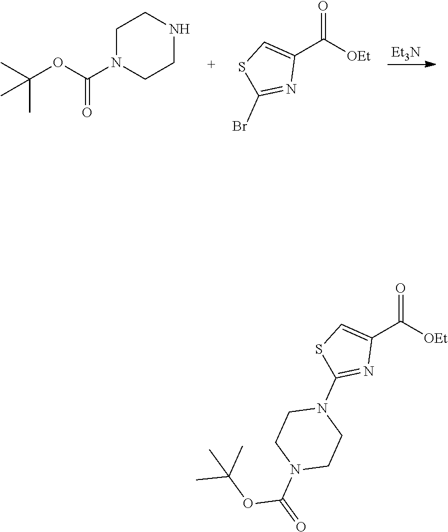 [SMILES: "Et3N" ; CC(C)(C)OC(=O)N1CCNCC1 ; CCOC(=O)c1csc(Br)n1 ; CCOC(=O)c1csc(N2CCN(C(=O)OC(C)(C)C)CC2)n1]

Tert-butyl piperazine-1-carboxylate (1.440 g, 7.73 mmole) was dissolved in DMA (8 mL). Ethyl 2-bromothiazole-4-carboxylate (1.998 g, 8.46 mmole) was added followed by triethylamine (1.30 mL, 9.28 mmole) and the mixture was heated to 80° C. for 18 hours, then was cooled to RT and diluted with ethyl acetate (50 mL). The mixture was washed sequentially with water (3×25 mL) and sat. aq. sodium chloride (1×25 mL). The organics were dried over magnesium sulfate, filtered and concentrated. The crude residue was purified by silica gel chromatography to provide ethyl 2-(4-(tert-butoxycarbonyl)piperazin-1-yl)thiazole-4-carboxylate as a white solid (1.877 g, 71%).

¹H NMR (400 MHz, CDCl₃): δ 7.47 (s, 1H), 4.36 (q, J=7.1 Hz, 2H), 3.61-3.47 (m, 8H), 1.48 (s, 9H), 1.37 (t, J=7.1 Hz, 3H).

### Tert-butyl (S)-4-(4-((3-hydroxy-1-methoxy-1-oxopropan-2-yl)carbamoyl)thiazol-2-yl)piperazine-1-carboxylate (47.2)

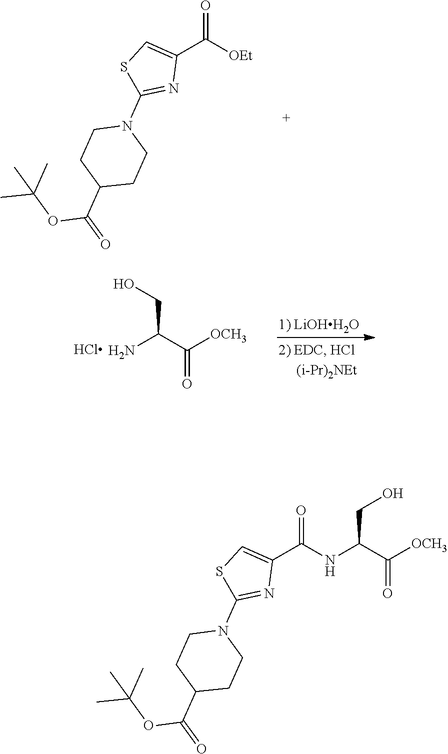

Ethyl 2-(4-(tert-butoxycarbonyl)piperazin-1-yl)thiazole-4-carboxylate (1.877 g, 5.50 mmole) was dissolved in 4/1/1 THF/methanol/water (11 mL). Lithium hydroxide monohydrate (0.344 g, 8.20 mmole) was added and the mixture was stirred at RT for 3 hours. Water (25 mL) was added and the solution was treated with 1N aq. HCl to pH=4. The mixture was extracted with ethyl acetate (6×10 mL) and the combined organics were dried over magnesium sulfate, filtered and concentrated. The residue was suspended in DCM (11 mL). L-Serine methyl ester hydrochloride (1.108 g, 7.12 mmole) was added followed by N,N-diisopropylethylamine (1.90 mL, 10.9 mmole) and EDC·HCl (1.372 g, 7.16 mmole). The mixture was stirred at RT for 18 hours and water (50 mL) was added. The two layers were separated and the aqueous layer was extracted with DCM (2×15 mL). The combined organics were dried over magnesium sulfate, filtered and concentrated. The crude residue was purified by silica gel chromatography to provide tert-butyl (S)-4-(4-((3-hydroxy-1-methoxy-1-oxopropan-2-yl)carbamoyl)thiazol-2-yl)piperazine-1-carboxylate as a white solid (1.229 g, 54%).

¹H NMR (400 MHz, CDCl₃): δ 7.99 (d, J=7.4 Hz, 1H), 7.43 (s, 1H), 4.80 (dt, J=7.6, 3.9 Hz, 1H), 4.09-4.01 (m, 2H), 3.82 (s, 3H), 3.57 (dd, J=6.6, 3.7 Hz, 4H), 3.49 (dd, J=6.2, 3.6 Hz, 4H), 2.82 (t, J=6.0 Hz, 1H), 1.49 (s, 9H).

### Methyl O-(tert-butyldimethylsilyl)-N-(2-(4-(2-(2-methoxyethoxy)acetyl)piperazin-1-yl)thiazole-4-carbonyl)-L-serinate (47.3)

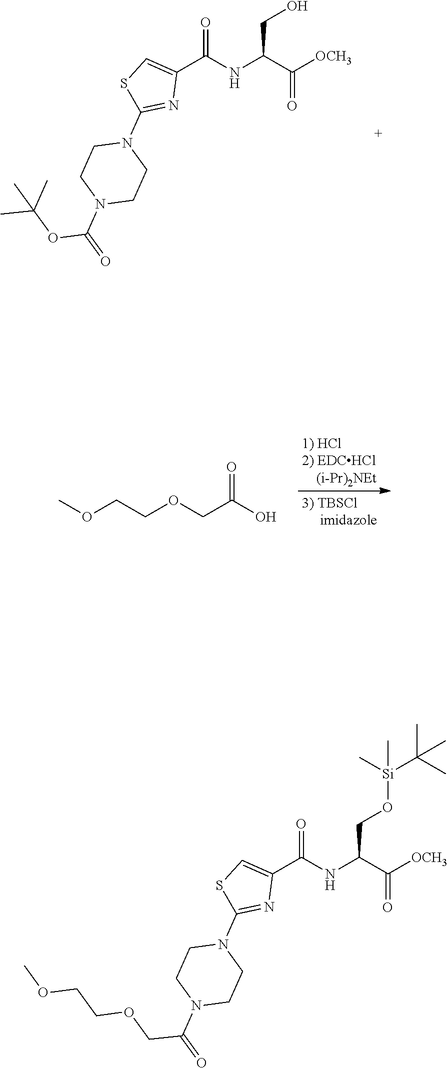

Tert-butyl (S)-4-(4-((3-hydroxy-1-methoxy-1-oxopropan-2-yl)carbamoyl)thiazol-2-yl)piperazine-1-carboxylate (1.229 g, 2.97 mmole) was dissolved in DCM (6 mL). HCl (4M solution in 1,4-dioxane, 3.00 mL, 12.0 mmole) was added and the mixture was stirred at RT for 3 hours, then was concentrated. The residue was suspended in DCM (6 mL) and 2-(2-methoxyethoxy)acetic acid (0.410 mL, 3.61 mmole) was added. N,N-Diisopropylethyl amine (1.05 mL, 6.03 mmole) and EDC·HCl (0.686 g, 3.58 mmole) were added and the mixture was stirred at RT for 18 hours. Water (25 mL) was added and the mixture was extracted with DCM (3×10 mL). The combined organics were dried over magnesium sulfate, filtered and concentrated. The residue was dissolved in DCM (6 mL) and imidazole (0.225 g, 3.30 mmole) and tert-butyldimethylchlorosilane (0.500 g, 3.32 mmole) were added. The solution was stirred at RT for 2 hours and water (25 mL) was added. The mixture was extracted with DCM (3×10 mL) and the combined organics were dried over magnesium sulfate, filtered and concentrated. The crude residue was purified by silica gel chromatography to provide methyl O-(tert-butyldimethylsilyl)-N-(2-(4-(2-(2-methoxyethoxy)acetyl)piperazin-1-yl)thiazole-4-carbonyl)-L-serinate as a yellow oil (0.904 g, 56%).

¹H NMR (400 MHz, CDCl₃): δ 7.92 (d, J=8.7 Hz, 1H), 7.44 (s, 1H), 4.79 (dt, J=8.7, 3.0 Hz, 1H), 4.26 (s, 2H), 4.20-4.14 (m, 1H), 3.93-3.84 (m, 1H), 3.79-3.72 (m, 2H), 3.77 (s, 3H), 3.73-3.65 (m, 4H), 3.61-3.55 (m, 2H), 3.53 (t, J=6.3 Hz, 4H), 3.39 (s, 3H), 0.89 (s, 9H), 0.05 (s, 3H), 0.03 (s, 3H).

### Methyl O-acetyl-N—(O-(tert-butyldimethylsilyl)-N-(2-(4-(2-(2-methoxyethoxy)acetyl)piperazin-1-yl)thiazole-4-carbonyl)-L-seryl)-L-serinate (47.4)

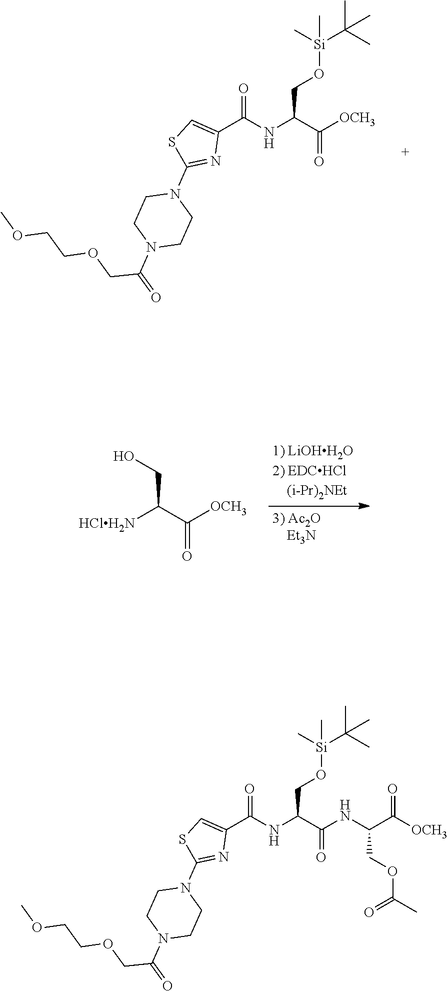

Methyl O-(tert-butyldimethylsilyl)-N-(2-(4-(2-(2-methoxyethoxy)acetyl)piperazin-1-yl)thiazole-4-carbonyl)-L-serinate (0.436 g, 0.800 mmole) was dissolved in 4/1/1 THF/methanol/water (1.6 mL). Lithium hydroxide monohydrate (0.061 g, 1.45 mmole) was added and the mixture was stirred at RT for 2 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was dissolved in DCM (1.6 mL) and L-serine methyl ester hydrochloride (0.168 g, 1.08 mmole) was added. N,N-Diisopropylethylamine (0.290 mL, 1.66 mmole) and EDC·HCl (0.221 g, 1.15 mmole) were added and the mixture was stirred at RT for 18 hours. Water (25 mL) was added and the mixture was extracted with DCM (3×10 mL). The combined organics were dried over magnesium sulfate, filtered and concentrated. The residue was dissolved in DCM (1.6 mL) and triethylamine (0.130 mL, 0.928 mmole) and acetic anhydride (0.086 mL, 0.910 mmole) were added. The mixture was stirred at RT for 3 hours and water (25 mL) was added. The mixture was extracted with DCM (3×10 mL) and the combined organics were dried over magnesium sulfate, filtered and concentrated. The crude residue was purified by silica gel chromatography to provide methyl O-acetyl-N—(O-(tert-butyldimethylsilyl)-N-(2-(4-(2-(2-methoxyethoxy)acetyl)piperazin-1-yl)thiazole-4-carbonyl)-L-seryl)-L-serinate as a colorless oil (0.120 g, 22%).

¹H NMR (400 MHz, CDCl₃): δ 7.96 (d, J=7.0 Hz, 1H), 7.49-7.42 (m, 2H), 4.87 (dt, J=7.7, 3.8 Hz, 1H), 4.58 (td, J=7.3, 3.7 Hz, 1H), 4.48 (dd, J=11.4, 3.9 Hz, 1H), 4.32 (dd, J=11.4, 3.6 Hz, 1H), 4.25 (s, 2H), 4.22-4.15 (m, 1H), 3.77 (s, 3H), 3.76-3.72 (m, 2H), 3.72-3.65 (m, 4H), 3.61-3.46 (m, 6H), 3.39 (s, 3H), 2.02 (s, 3H), 0.93 (s, 9H), 0.15 (s, 3H), 0.14 (s, 3H).

### Methyl 2-(2-(2-(4-(2-(2-methoxyethoxy)acetyl)piperazin-1-yl)thiazole-4-carboxamido)acrylamido)acrylate (47)

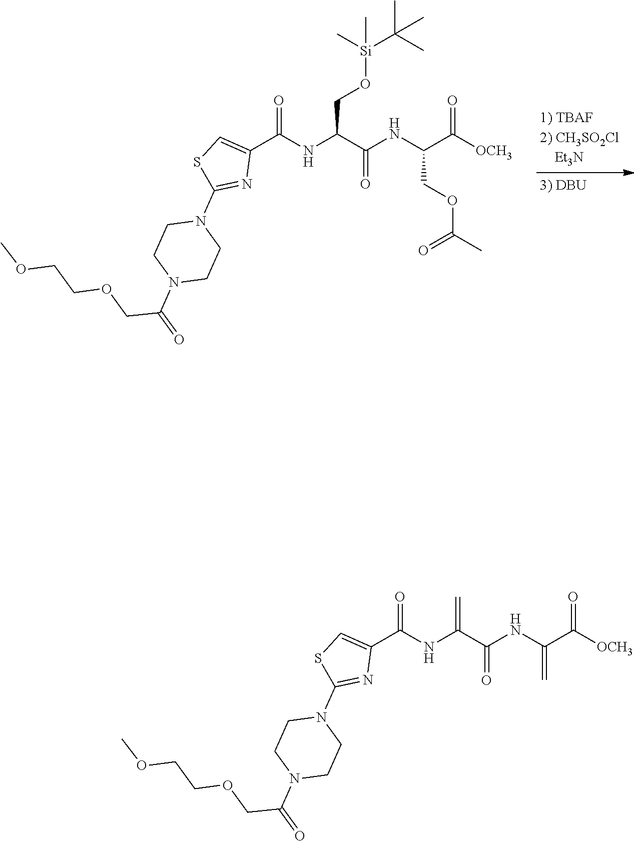 [SMILES: COCCOCC(=O)N1CCN(c2nc(C(=O)N[C@@H](CO[Si](C)(C)C(C)(C)C)C(=O)N[C@@H](COC(C)=O)C(=O)OC)cs2)CC1 ; C=C(NC(=O)c1csc(N2CCN(C(=O)COCCOC)CC2)n1)C(=O)NC(=C)C(=O)OC]

Methyl O-acetyl-N—(O-(tert-butyldimethylsilyl)-N-(2-(4-(2-(2-methoxyethoxy)acetyl)piperazin-1-yl)thiazole-4-carbonyl)-L-seryl)-L-serinate (0.120 g, 0.178 mmole) was dissolved in THF (1.8 mL). TBAF (1M solution in THF, 0.450 mL, 0.450 mmole) was added and the solution was stirred at RT for 2 hours. Water (25 mL) and sat. aq. sodium chloride (10 mL) were added and the mixture was extracted with ethyl acetate (3×10 mL). The combined organics were dried over magnesium sulfate, filtered and concentrated. The residue was dissolved in DCM (1.8 mL) and cooled to 0° C. Triethylamine (0.040 mL, 0.285 mmole) and methanesulfonyl chloride (0.026 mL, 0.336 mmole) were added and the mixture was stirred at 0° for 60 minutes and water (25 mL) was added. The mixture was extracted with DCM (3×10 mL) and the combined organics were dried over magnesium sulfate, filtered and concentrated. The residue was dissolved in THF (2 mL) and cooled to 0° C. DBU (0.040 mL, 0.267 mmole) was added and the mixture was stirred at 0° for 45 minutes. Water (25 mL) was added and the mixture was extracted with DCM (3×10 mL). The combined organics were dried over magnesium sulfate, filtered and concentrated. The crude residue was purified by silica gel chromatography to provide methyl 2-(2-(2-(4-(2-(2-methoxyethoxy)acetyl)piperazin-1-yl)thiazole-4-carboxamido)acrylamido)acrylate as a pale yellow solid (0.026 g, 30%).

¹H NMR (400 MHz, CDCl₃): δ 9.74 (s, 1H), 8.52 (s, 1H), 7.48 (s, 1H), 6.73 (d, J=2.1 Hz, 1H), 6.66 (s, 1H), 6.01 (d, J=1.3 Hz, 1H), 5.44 (t, J=1.9 Hz, 1H), 4.26 (s, 2H), 3.90 (s, 3H), 3.77 (s, 2H), 3.74-3.67 (m, 4H), 3.63-3.56 (m, 4H), 3.49 (d, J=12.0 Hz, 2H), 3.39 (s, 3H).

### Compound 48: Methyl 2-(2-(2-(4-(6-chlorohexanamido)phenyl)thiazole-4-carboxamido)acrylamido)acrylate

### Tert-butyl (4-carbamothioylphenyl)carbamate (48.1)

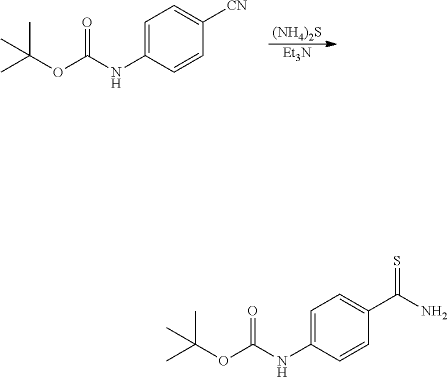 [SMILES: "Et3N" ; CC(C)(C)OC(=O)Nc1ccc(C#N)cc1 ; "(NH4)2S" ; CC(C)(C)OC(=O)Nc1ccc(C(N)=S)cc1]

Tert-butyl (4-cyanophenyl)carbamate (5.024 g, 23.0 mmole) was dissolved in pyridine (23 mL). Triethylamine (3.60 mL, 25.7 mmole) and ammonium sulfide (40% aqueous solution, 4.70 mL, 27.5 mmole) were added and the mixture was heated to 50° C. for 24 hours, then was cooled to RT and concentrated. The residue was dissolved in ethyl acetate (50 mL) and the solution was washed sequentially with 1N aq. HCl (2×25 mL), water (1×25 mL) and sat. aq. sodium chloride (1×25 mL). The organics were dried over magnesium sulfate, filtered and concentrated. The residue was suspended in diethyl ether (15 mL) and the solid was collected by filtration, then allowed to air dry to provide tert-butyl (4-carbamothioylphenyl)carbamate as a yellow solid (4.826 g, 83%).

¹H NMR (400 MHz, d₆-DMSO): δ 9.64 (d, J=4.5 Hz, 2H), 9.30 (s, 1H), 7.93-7.82 (m, 2H), 7.51-7.40 (m, 2H), 1.48 (s, 9H).

### Ethyl 2-(4-aminophenyl)thiazole-4-carboxylate (48.2)

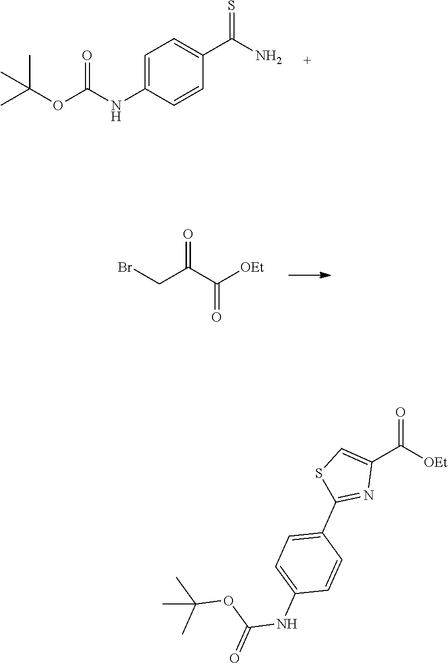 [SMILES: CC(C)(C)OC(=O)Nc1ccc(C(N)=S)cc1 ; CCOC(=O)C(=O)CBr ; CCOC(=O)c1csc(-c2ccc(NC(=O)OC(C)(C)C)cc2)n1]

Tert-butyl (4-carbamothioylphenyl)carbamate (4.826 g, 19.1 mmole) was dissolved in ethanol (38 mL). Ethyl bromopyruvate (2.90 mL, 23.1 mmole) was added and the mixture was heated to 80° C. for 4 hours. The mixture was cooled to RT and concentrated. The residue was suspended in DCM (38 mL) and triethylamine (5.40 mL, 38.5 mmole) and di-tert-butyl dicarbonate (4.608 g, 21.1 mmole) were added. The mixture was stirred at RT for 18 hours and water (75 mL) was added. The two layers were separated and the aqueous layer was extracted with DCM (2×15 mL). The combined organics were dried over magnesium sulfate, filtered and concentrated. The crude residue was purified by silica gel chromatography to provide ethyl 2-(4-((tert-butoxycarbonyl)amino)phenyl)thiazole-4-carboxylate as a yellow solid (0.983 g, 15%) and ethyl 2-(4-aminophenyl)thiazole-4-carboxylate (Compound 48.2) as an orange solid (2.646 g, 56%).

¹H NMR (400 MHz, CDCl₃): δ 8.03 (s, 1H), 7.85-7.78 (m, 2H), 6.74-6.65 (m, 2H), 4.43 (q, J=7.1 Hz, 2H), 3.97 (s, 2H), 1.42 (t, J=7.1 Hz, 3H).

### Ethyl 2-(4-(6-chlorohexanamido)phenyl)thiazole-4-carboxylate (48.3)

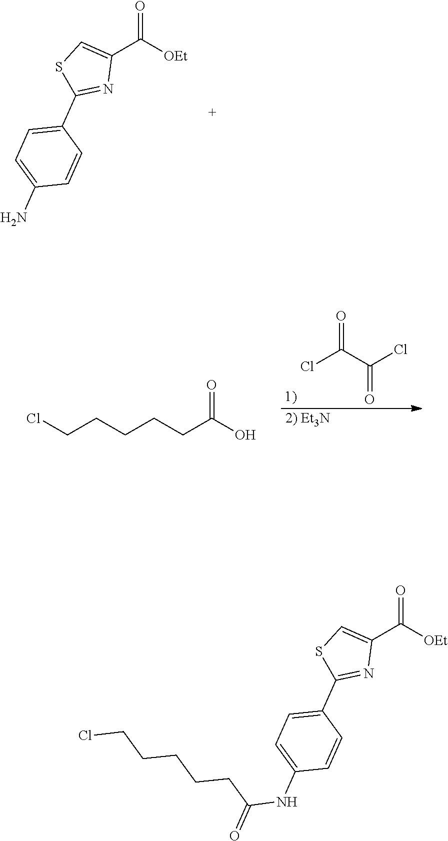

6-Chlorohexanoic acid 1.573 g, 10.4 mmole) was dissolved in DCM (16 mL) and DMF (1 drop) was added. Oxalyl chloride (0.910 mL, 10.4 mmole) was added dropwise (gas evolution was observed) and the solution was stirred at RT for 3 hours, then was concentrated.
Ethyl 2-(4-aminophenyl)thiazole-4-carboxylate (1.986 g, 8.00 mmole) was dissolved in DCM (8 mL) and cooled to 0° C. Triethylamine (1.70 mL, 12.1 mmole) was added followed by a solution of the acid chloride generated above in DCM (8 mL). The resulting mixture was allowed to warm to RT and stir for 18 hours. Water (50 mL) was added and the two layers were separated. The aqueous layer was extracted with DCM (2×15 mL) and the combined organics were dried over magnesium sulfate, filtered and concentrated. The crude residue was purified by silica gel chromatography to provide ethyl 2-(4-(6-chlorohexanamido)phenyl)thiazole-4-carboxylate as a yellow solid (2.441 g, 80%).

¹H NMR (400 MHz, CDCl₃): δ 8.12 (s, 1H), 8.01-7.92 (m, 2H), 7.68 (d, J=8.4 Hz, 2H), 7.59 (s, 1H), 4.45 (q, J=7.1 Hz, 2H), 3.55 (t, J=6.6 Hz, 2H), 2.42 (t, J=7.4 Hz, 2H), 1.79 (tdd, J=15.4, 10.9, 6.9 Hz, 4H), 1.60-1.47 (m, 2H), 1.43 (t, J=7.1 Hz, 3H).

### Methyl O-(tert-butyldimethylsilyl)-N-(2-(4-(6-chlorohexanamido)phenyl)thiazole-4-carbonyl)-L-serinate (48.4)

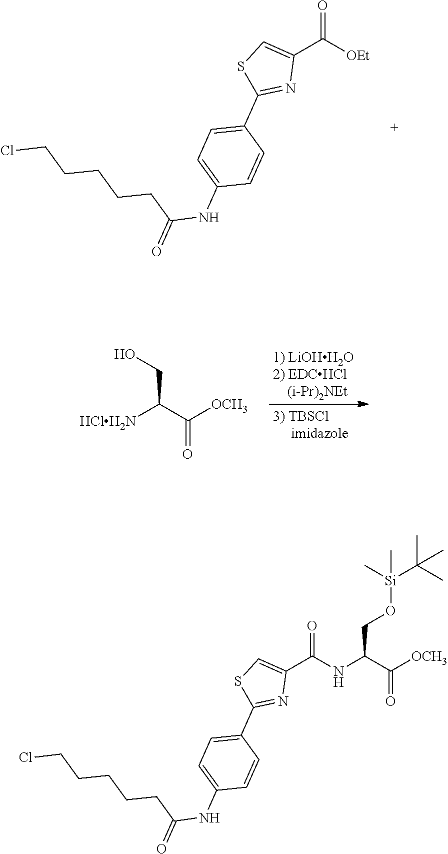

Ethyl 2-(4-(6-chlorohexanamido)phenyl)thiazole-4-carboxylate (2.441 g, 6.41 mmole) was dissolved in 4/1/1 THF/methanol/water (13 mL). Lithium hydroxide monohydrate (0.414 g, 9.87 mmole) was added and the mixture was stirred at RT for 2 hours. Water (50 mL) was added and the solution was treated with 1N aq. HCl to pH=4. The mixture was extracted with ethyl acetate (3×15 mL). The combined organics were filtered to remove the precipitated acid, and the filtrate was dried over magnesium sulfate, filtered and concentrated. The residue was combined with the filtered solid and suspended in DCM (13 mL). L-Serine methyl ester hydrochloride (1.306 g, 8.39 mmole) was added followed by N,N-diisopropylethylamine (2.30 mL, 13.2 mmole) and EDC·HCl (1.597 g, 8.33 mmole). The mixture was stirred at RT for 2 days and water (50 mL) was added. The two layers were separated and the aqueous layer was extracted with DCM (2×15 mL). The combined organics were dried over magnesium sulfate, filtered and concentrated. The residue was dissolved in DCM (13 mL) and imidazole (0.479 g, 7.04 mmole) and tert-butyldimethylchlorosilane (1.059 g, 7.03 mmole) were added. The mixture was stirred at RT for 3 hours and water (50 mL) was added. The two layers were separated and the aqueous layer was extracted with DCM (2×10 mL). The combined organics were dried over magnesium sulfate, filtered and concentrated. The crude residue was purified by silica gel chromatography to provide methyl O-(tert-butyldimethylsilyl)-N-(2-(4-(6-chlorohexanamido)phenyl)thiazole-4-carbonyl)-L-serinate as a yellow oil (1.991 g, 55%).

¹H NMR (400 MHz, CDCl₃): δ 8.21 (d, J=8.7 Hz, 1H), 8.04 (s, 1H), 7.93-7.84 (m, 2H), 7.67-7.56 (m, 3H), 4.85 (dt, J=8.7, 3.0 Hz, 1H), 4.22 (dd, J=10.0, 2.7 Hz, 1H), 3.95 (dd, J=10.1, 3.4 Hz, 1H), 3.79 (s, 3H), 3.56 (t, J=6.6 Hz, 2H), 2.41 (t, J=7.4 Hz, 2H), 1.89-1.72 (m, 4H), 1.60-1.50 (m, 2H), 0.92 (s, 9H), 0.08 (s, 3H), 0.06 (s, 3H).

### Methyl O-acetyl-N—(O-(tert-butyldimethylsilyl)-N-(2-(4-(6-chlorohexanamido)phenyl)thiazole-4-carbonyl)-L-seryl)-L-serinate (48.5)

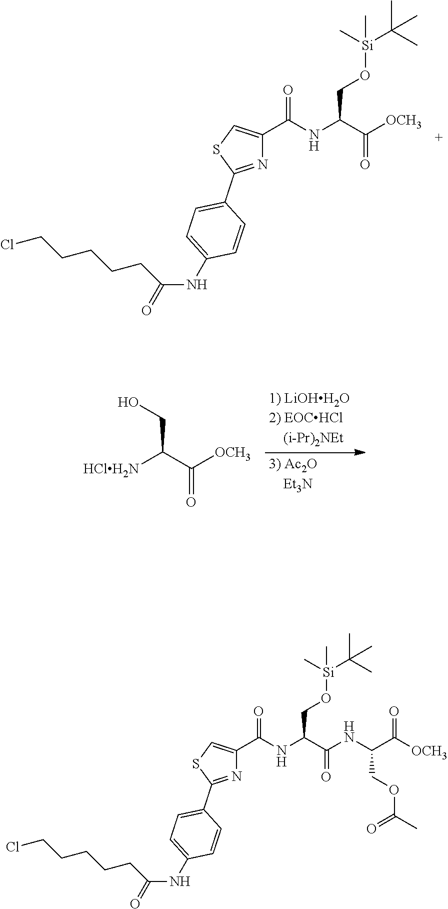

Methyl O-(tert-butyldimethylsilyl)-N-(2-(4-(6-chlorohexanamido)phenyl)thiazole-4-carbonyl)-L-serinate (1.991 g, 3.50 mmole) was dissolved in 4/1/1 THF/methanol/water (7 mL). Lithium hydroxide monohydrate (0.223 g, 5.31 mmole) was added and the mixture was stirred at RT for 4 hours. Water (50 mL) was added and the solution was treated with 1N aq. HCl to pH=4. The mixture was extracted with ethyl acetate (3×15 mL) and the combined organics were dried over magnesium sulfate, filtered and concentrated. The residue was dissolved in DCM (7 mL) and L-serine methyl ester hydrochloride (0.707 g, 4.54 mmole) was added. N,N-Diisopropylethylamine (1.25 mL, 7.18 mmole) and EDC·HCl (0.878 g, 4.58 mmole) were added and the mixture was stirred at RT for 18 hours. Water (25 mL) was added and the mixture was extracted with DCM (3×10 mL). The combined organics were dried over magnesium sulfate, filtered and concentrated. The residue was dissolved in DCM (7 mL) and triethylamine (0.540 mL, 3.85 mmole) and acetic anhydride (0.370 mL, 3.91 mmole) were added. The mixture was stirred at RT for 3 hours and water (25 mL) was added. The mixture was extracted with DCM (3×10 mL) and the combined organics were dried over magnesium sulfate, filtered and concentrated. The crude residue was purified by silica gel chromatography to provide methyl O-acetyl-N—(O-(tert-butyldimethylsilyl)-N-(2-(4-(6-chlorohexanamido)phenyl)thiazole-4-carbonyl)-L-seryl)-L-serinate as a pale yellow oil (1.004 g, 41%).

¹H NMR (400 MHz, CDCl₃): δ 8.25 (d, J=7.0 Hz, 1H), 8.07 (d, J=3.7 Hz, 1H), 7.94-7.87 (m, 2H), 7.63 (d, J=8.5 Hz, 2H), 7.53 (d, J=10.2 Hz, 1H), 7.48 (d, J=7.7 Hz, 1H), 4.89 (tt, J=6.2, 3.7 Hz, 1H), 4.67 (td, J=7.2, 3.7 Hz, 1H), 4.53-4.46 (m, 1H), 4.34 (dd, J=11.4, 3.6 Hz, 1H), 4.22 (dt, J=9.9, 3.8 Hz, 1H), 3.84-3.79 (m, 1H), 3.77 (d, J=3.3 Hz, 3H), 3.56 (t, J=6.6 Hz, 2H), 2.42 (t, J=7.4 Hz, 2H), 2.01 (s, 3H), 1.88-1.72 (m, 4H), 1.61-1.49 (m, 2H), 0.96 (s, 9H), 0.16 (s, 3H), 0.15 (s, 3H).

### Methyl 2-(2-(2-(4-(6-chlorohexanamido)phenyl)thiazole-4-carboxamido)acrylamido)acrylate (48)

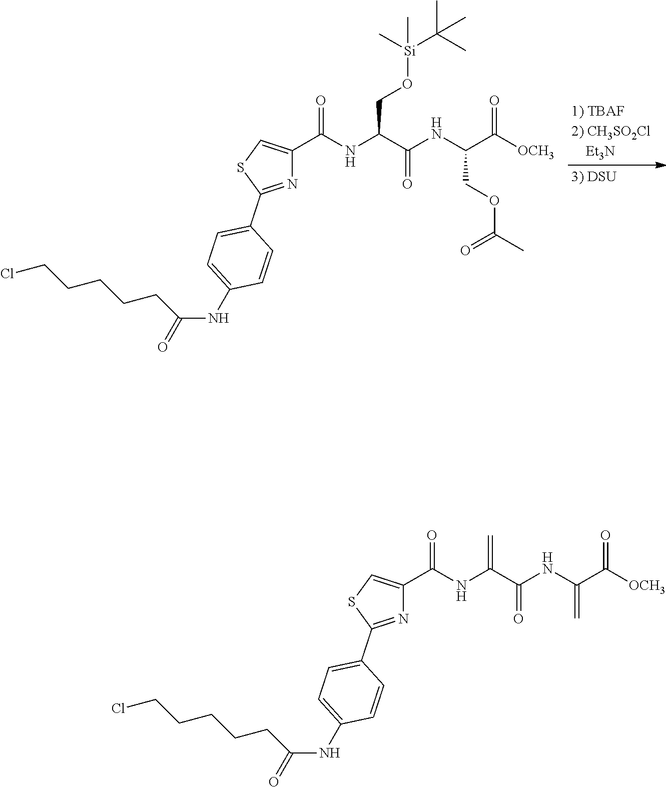 [SMILES: COC(=O)[C@H](COC(C)=O)NC(=O)[C@H](CO[Si](C)(C)C(C)(C)C)NC(=O)c1csc(-c2ccc(NC(=O)CCCCCCl)cc2)n1 ; C=C(NC(=O)c1csc(-c2ccc(NC(=O)CCCCCCl)cc2)n1)C(=O)NC(=C)C(=O)OC]

Methyl O-acetyl-N—(O-(tert-butyldimethylsilyl)-N-(2-(4-(6-chlorohexanamido)phenyl)thiazole-4-carbonyl)-L-seryl)-L-serinate (TLS-025-5-1) was dissolved in THF. TBAF (1M solution in THF) was added and the solution was stirred at RT for 2 hours. Water (25 mL) was added and the mixture was extracted with ethyl acetate (3×10 mL). The combined organics were dried over magnesium sulfate, filtered and concentrated. The residue was dissolved in DCM and cooled to 0° C. Triethylamine and methanesulfonyl chloride were added and the mixture was stirred at 0° C. for 90 minutes. Water (25 mL) was added and the mixture was extracted with DCM (3×10 mL). The combined organics were dried over magnesium sulfate, filtered and concentrated. The residue was dissolved in THF (2.9 mL) and cooled to 0° C. DBU was added and the mixture was stirred at 0° C. for 60 minutes. Water (25 mL) was added and the mixture was extracted with ethyl acetate (3×10 mL). The combined organics were dried over magnesium sulfate, filtered and concentrated. The crude residue was purified by silica gel chromatography (Isco CombiPrep, 12 g RediSep column, 20-50% ethyl acetate/hexane gradient) to provide methyl 2-(2-(2-(4-(6-chlorohexanamido)phenyl)thiazole-4-carboxamido)acrylamido)acrylate (TLS-025-11-1) as a white solid (0.356 g, 49%).

### Compound 49: Methyl 2-(2-(2-(4-(13,13-dimethyl-3,11-dioxo-4,7,12-trioxa-2,10-diazatetradecyl)phenyl)thiazole-4-carboxamido)acrylamido)acrylate

### Ethyl 2-(4-(13,13-dimethyl-3,11-dioxo-4,7,12-trioxa-2,10-diazatetradecyl)phenyl)thiazole-4-carboxylate (49.1)

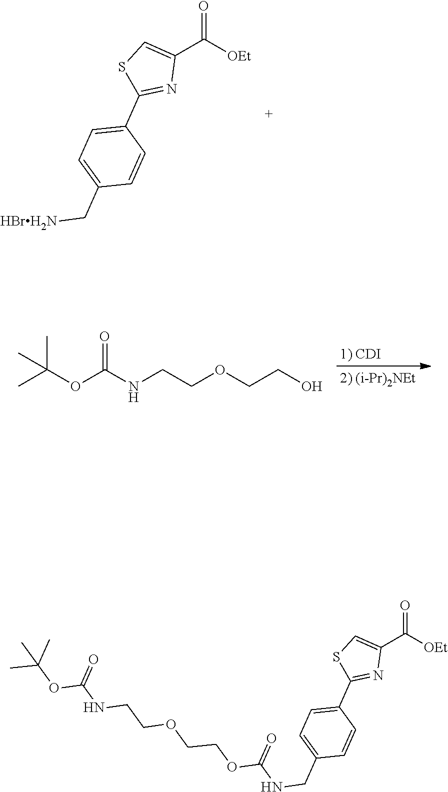

Tert-butyl (2-(2-hydroxyethoxy)ethyl)carbamate (0.496 g, 2.42 mmole) was dissolved in THF (4.4 mL). CDI (0.418 g, 2.58 mmole) was added slowly and the solution was stirred at RT for 4 hours, then was diluted with ethyl acetate (10 mL). Water (25 mL) was added and the two layers were separated. The aqueous layer was extracted with ethyl acetate (2×10 mL) and the combined organics were dried over magnesium sulfate, filtered and concentrated. The residue was dissolved in DMF (4.4 mL) and ethyl 2-(4-(aminomethyl)phenyl)thiazole-4-carboxylate hydrobromide (Example 45.1, 0.752 g, 2.19 mmole) was added. N,N-Diisopropylethylamine (0.460 mL, 2.64 mmole) was added and the mixture was warmed to 50° C. for 18 hours, then was cooled to RT and diluted with ethyl acetate (25 mL). The mixture was washed with water (3×15 mL) and the organics were dried over magnesium sulfate, filtered and concentrated. The crude residue was purified by silica gel chromatography to provide ethyl 2-(4-(13,13-dimethyl-3,11-dioxo-4,7,12-trioxa-2,10-diazatetradecyl)phenyl)thiazole-4-carboxylate as an orange solid (0.873 g, 81%).

¹H NMR (400 MHz, CDCl₃): δ 8.15 (s, 1H), 7.97 (d, J=8.3 Hz, 2H), 7.38 (d, J=8.0 Hz, 2H), 5.33 (s, 1H), 4.98 (s, 1H), 4.50-4.38 (m, 4H), 4.31-4.23 (m, 2H), 3.71-3.62 (m, 2H), 3.62-3.50 (m, 2H), 3.32 (d, J=5.8 Hz, 2H), 1.49-1.38 (m, 12H).

### Methyl O-(tert-butyldimethylsilyl)-N-(2-(4-(13,13-dimethyl-3,11-dioxo-4,7,12-trioxa-2,10-diazatetradecyl)phenyl)thiazole-4-carbonyl)-L-serinate (49.2)

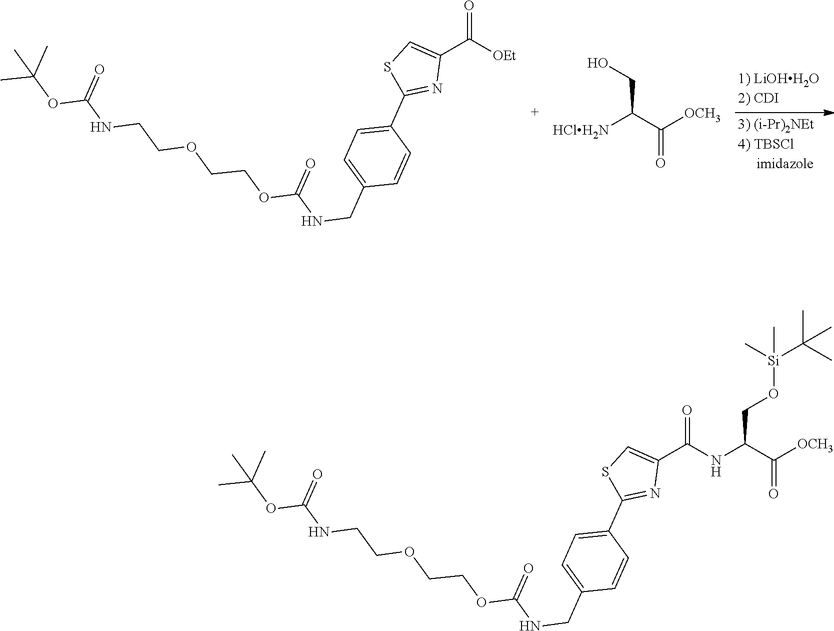

Ethyl 2-(4-(13,13-dimethyl-3,11-dioxo-4,7,12-trioxa-2,10-diazatetradecyl)phenyl)thiazole-4-carboxylate (0.873 g, 1.77 mmole) was dissolved in 4/1/1 THF/methanol/water (3.5 mL). Lithium hydroxide monohydrate (0.111 g, 2.65 mmole) was added and the mixture was stirred at RT for 4 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was suspended in DCM (3.5 mL) and CDI (0.317 g, 1.95 mmole) was added portionwise (mixture became homogeneous and gas evolution was observed). The solution was stirred for 60 minutes and L-serine methyl ester hydrochloride (0.365 g, 2.35 mmole) and N,N-diisopropylethylamine (0.620 mL, 3.56 mmole) were added. The mixture was stirred at RT for 2½ days and imidazole (0.136 g, 2.00 mmole) and tert-butyldimethylchlorosilane (0.307 g, 2.04 mmole) were added. The mixture was stirred at RT for 4 hours and water (25 mL) was added. The mixture was extracted with DCM (3×10 mL) and the combined organics were dried over magnesium sulfate, filtered and concentrated. The crude residue was purified by silica gel chromatography to provide methyl O-(tert-butyldimethylsilyl)-N-(2-(4-(13,13-dimethyl-3,11-dioxo-4,7,12-trioxa-2,10-diazatetradecyl)phenyl)thiazole-4-carbonyl)-L-serinate as a colorless oil (0.614 g, 51%).

¹H NMR (400 MHz, CDCl₃): δ 8.21 (d, J=8.7 Hz, 1H), 8.10 (s, 1H), 7.97-7.88 (m, 2H), 7.42-7.33 (m, 2H), 5.31 (s, 1H), 4.97 (s, 1H), 4.86 (dt, J=8.7, 3.1 Hz, 1H), 4.44 (d, J=6.1 Hz, 2H), 4.32-4.24 (m, 2H), 4.21 (dd, J=10.1, 2.7 Hz, 1H), 4.00-3.91 (m, 1H), 3.79 (s, 3H), 3.71-3.64 (m, 2H), 3.55 (q, J=7.6, 6.5 Hz, 2H), 3.38-3.25 (m, 2H), 1.43 (s, 9H), 0.92 (s, 8H), 0.08 (s, 3H), 0.06 (s, 3H).

### Methyl O-acetyl-N—(O-(tert-butyldimethylsilyl)-N-(2-(4-(13,13-dimethyl-3,11-dioxo-4,7,12-trioxa-2,10-diazatetradecyl)phenyl)thiazole-4-carbonyl)-L-seryl)-L-serinate (49.3)

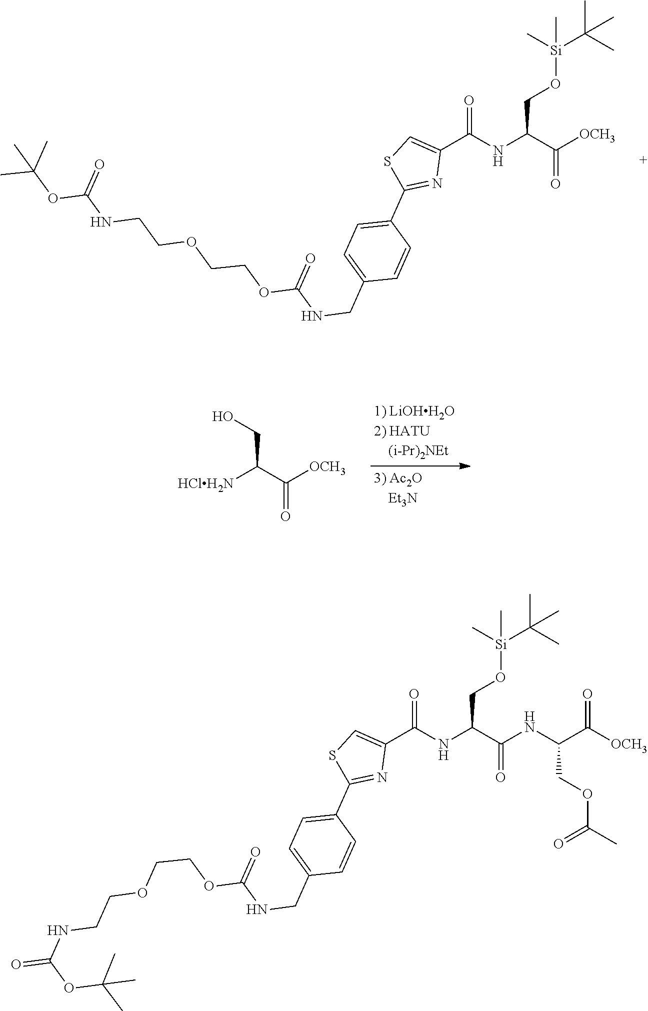

Methyl O-(tert-butyldimethylsilyl)-N-(2-(4-(13,13-dimethyl-3,11-dioxo-4,7,12-trioxa-2,10-diazatetradecyl)phenyl)thiazole-4-carbonyl)-L-serinate (0.614 g, 0.902 mmole) was dissolved in 4/1/1 THF/methanol/water (2 mL). Lithium hydroxide monohydrate (0.066 g, 1.57 mmole) was added and the mixture was stirred at RT for 90 minutes. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was dissolved in DCM (2 mL) and L-serine methyl ester hydrochloride (0.187 g, 1.20 mmole) was added. N,N-Diisopropylethylamine (0.320 mL, 1.84 mmole) and HATU (0.410 g, 1.08 mmole) were added and the mixture was stirred at RT for 18 hours. Water (25 mL) was added and the mixture was extracted with DCM (3×10 mL). The combined organics were dried over magnesium sulfate, filtered and concentrated. The residue was dissolved in DCM (2 mL). Triethylamine (0.140 mL, 0.999 mmole) and acetic anhydride (0.094 mL, 0.994 mmole) were added and the mixture was stirred at RT for 4 hours. Water (25 mL) was added and the mixture was extracted with DCM (3×10 mL). The combined organics were dried over magnesium sulfate, filtered and concentrated. The crude residue was purified by silica gel chromatography to provide methyl O-acetyl-N—(O-(tert-butyldimethylsilyl)-N-(2-(4-(13,13-dimethyl-3,11-dioxo-4,7,12-trioxa-2,10-diazatetradecyl)phenyl)thiazole-4-carbonyl)-L-seryl)-L-serinate as a pale yellow oil (0.530 g, 73%).

¹H NMR (400 MHz, CDCl₃): δ 8.26 (dd, J=7.2, 2.5 Hz, 1H), 8.24-8.19 (m, 1H), 8.11 (d, J=2.5 Hz, 1H), 7.94 (dd, J=8.4, 1.8 Hz, 2H), 7.48 (d, J=7.8 Hz, 1H), 7.38 (d, J=8.0 Hz, 2H), 6.54-6.45 (m, 1H), 4.97 (s, 1H), 4.90 (dq, J=7.0, 3.5 Hz, 1H), 4.67 (td, J=7.3, 3.7 Hz, 1H), 4.50 (dd, J=11.4, 3.9 Hz, 1H), 4.43 (d, J=6.3 Hz, 2H), 4.34 (dd, J=11.4, 3.6 Hz, 1H), 4.30-4.25 (m, 2H), 4.25-4.18 (m, 1H), 3.85-3.79 (m, 1H), 3.77 (d, J=4.3 Hz, 3H), 3.68 (dd, J=5.7, 3.6 Hz, 2H), 3.56 (t, J=5.3 Hz, 2H), 3.33 (d, J=6.0 Hz, 2H), 2.02 (s, 3H), 1.43 (s, 9H), 0.96 (s, 9H), 0.17 (s, 3H), 0.15 (s, 3H).

### Methyl 2-(2-(2-(4-(13,13-dimethyl-3,11-dioxo-4,7,12-trioxa-2,10-diazatetradecyl)phenyl)thiazole-4-carboxamido)acrylamido)acrylate (49)

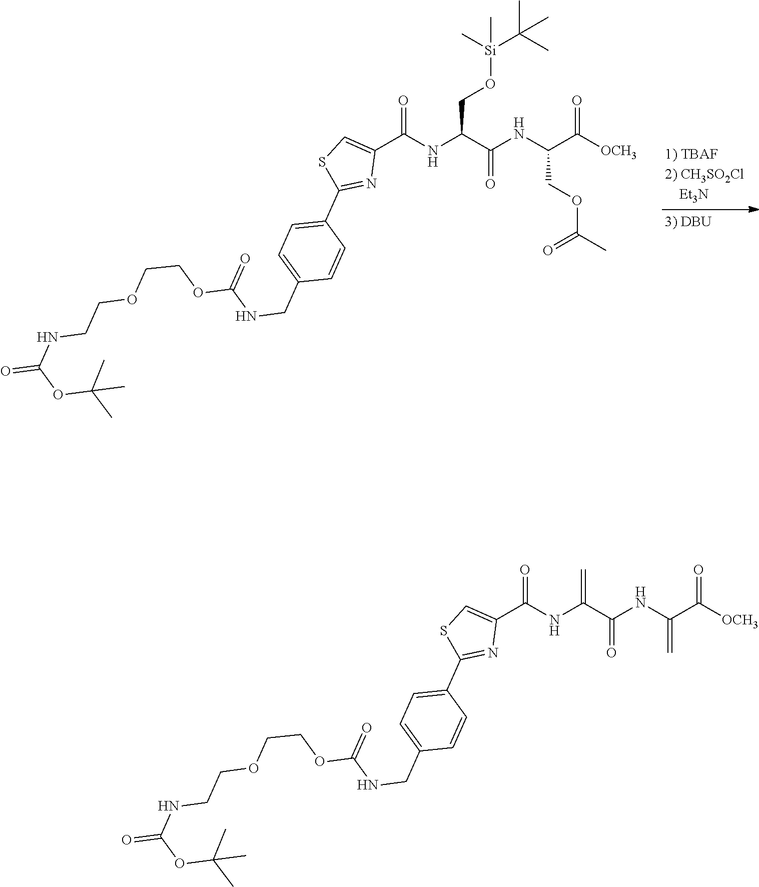 [SMILES: COC(=O)[C@H](COC(C)=O)NC(=O)[C@H](CO[Si](C)(C)C(C)(C)C)NC(=O)c1csc(-c2ccc(CNC(=O)OCCOCCNC(=O)OC(C)(C)C)cc2)n1 ; C=C(NC(=O)c1csc(-c2ccc(CNC(=O)OCCOCCNC(=O)OC(C)(C)C)cc2)n1)C(=O)NC(=C)C(=O)OC]

Methyl O-acetyl-N—(O-(tert-butyldimethylsilyl)-N-(2-(4-(13,13-dimethyl-3,11-dioxo-4,7,12-trioxa-2,10-diazatetradecyl)phenyl)thiazole-4-carbonyl)-L-seryl)-L-serinate (0.530 g, 0.654 mmoles) was dissolved in THF (1.3 mL). TBAF (1M solution in THF, 1.65 mL, 1.65 mmole) was added and the solution was stirred at RT for 2 hours. Water (25 mL) was added and the mixture was extracted with ethyl acetate (3×10 mL). The combined organics were dried over magnesium sulfate, filtered and concentrated. The residue was dissolved in DCM (2.6 mL) and cooled to 0° C. Triethylamine (0.140 mL, 0.999 mmole) and methanesulfonyl chloride (0.076 mL, 0.982 mmole) were added and the mixture was stirred at 0° for 90 minutes. Water (25 mL) was added and the mixture was extracted with DCM (3×10 mL). The combined organics were dried over magnesium sulfate, filtered and concentrated. The residue was dissolved in THF (1.3 mL) and cooled to 0° C. DBU (0.150 mL, 1.00 mmole) was added and the mixture was stirred at 0° for 60 minutes. Water (25 mL) was added and the mixture was extracted with ethyl acetate (3×10 mL). The combined organics were dried over magnesium sulfate, filtered and concentrated. The crude residue was purified by silica gel chromatography to provide methyl 2-(2-(2-(4-(13,13-dimethyl-3,11-dioxo-4,7,12-trioxa-2,10-diazatetradecyl)phenyl)thiazole-4-carboxamido)acrylamido)acrylate as a white solid (0.121 g, 30%).

¹H NMR (400 MHz, CDCl₃): δ 10.01 (s, 1H), 8.55 (s, 1H), 8.14 (s, 1H), 8.03-7.93 (m, 2H), 7.40 (d, J=8.2 Hz, 2H), 6.78 (d, J=2.2 Hz, 1H), 6.71 (s, 1H), 6.03 (d, J=1.3 Hz, 1H), 5.50 (t, J=1.9 Hz, 1H), 5.27 (s, 1H), 4.96 (s, 1H), 4.44 (d, J=6.1 Hz, 2H), 4.33-4.23 (m, 2H), 3.91 (s, 3H), 3.73-3.62 (m, 2H), 3.56 (t, J=5.2 Hz, 2H), 3.39-3.23 (m, 2H), 1.43 (s, 9H).

### Compound 50: Methyl 2-(2-(2-(4-(((((6-azidohexyl)oxy)carbonyl)amino)methyl)phenyl)thiazole-4-carboxamido)acrylamido)acrylate

### Ethyl 2-(4-(((((6-azidohexyl)oxy)carbonyl)amino)methyl)phenyl)thiazole-4-carboxylate (50.1)

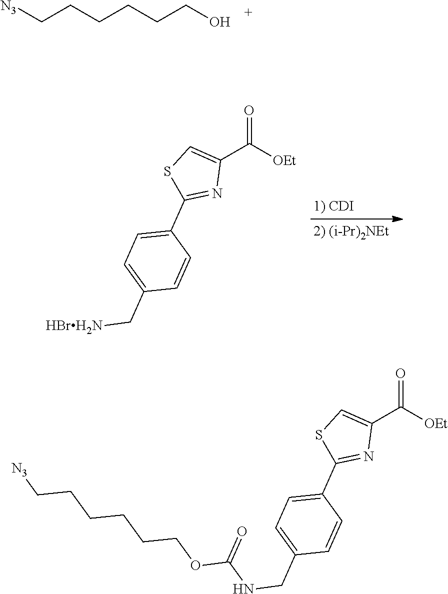

6-Azidohexan-1-ol (0.794 g, 5.55 mmole) was dissolved in THF (11 mL). CDI (0.904 g, 5.58 mmole) was added slowly and the solution was stirred at RT for 18 hours. Water (25 mL) was added and the mixture was extracted with ethyl acetate (3×10 mL). The combined organics were dried over magnesium sulfate, filtered and concentrated. The residue was dissolved in DMF (6 mL) and ethyl 2-(4-(aminomethyl)phenyl)thiazole-4-carboxylate hydrobromide (Example 45.1, 1.992 g, 5.80 mmole) was added. N,N-Diisopropylethylamine (1.20 mL, 6.89 mmole) was added and the mixture was warmed to 50° C. and stirred for 5 hours, then was cooled to RT and diluted with ethyl acetate (50 mL). The mixture was washed with water (3×20 mL) and the organics were dried over magnesium sulfate, filtered and concentrated. The crude residue was purified by silica gel chromatography to provide ethyl 2-(4-(((((6-azidohexyl)oxy)carbonyl)amino)methyl)phenyl)thiazole-4-carboxylate as a yellow solid (1.125 g, 47%).

¹H NMR (400 MHz, CDCl₃): δ 8.15 (s, 1H), 8.02-7.94 (m, 2H), 7.37 (d, J=8.2 Hz, 2H), 5.10 (s, 1H), 4.50-4.35 (m, 4H), 4.15-4.06 (m, 2H), 3.28 (dt, J=10.9, 6.9 Hz, 2H), 1.72-1.52 (m, 4H), 1.42 (q, J=6.3, 5.5 Hz, 7H).

### Methyl N-(2-(4-(((((6-azidohexyl)oxy)carbonyl)amino)methyl)phenyl)thiazole-4-carbonyl)-O-(tert-butyldimethylsilyl)-L-serinate (50.2)

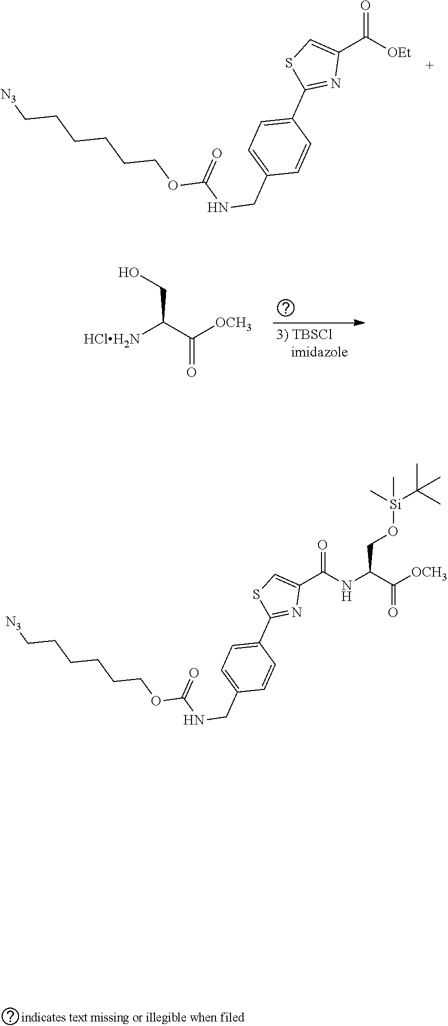

Ethyl 2-(4-(((((6-azidohexyl)oxy)carbonyl)amino)methyl)phenyl)thiazole-4-carboxylate (1.125 g, 2.61 mmole) was dissolved in 4/1/1 THF/methanol/water (5.2 mL). Lithium hydroxide monohydrate (0.171, 4.08 mmole) was added and the mixture was stirred at RT for 3 hours. Water (25 mL) was added and the mixture was treated with 1N aq. HCl to pH=4. The mixture was extracted with ethyl acetate (3×15 mL) and the combined organics were dried over magnesium sulfate, filtered and concentrated. The residue was dissolved in DCM (5.2 mL) and L-serine methyl ester hydrochloride (0.525 g, 3.37 mmole) was added. NN-Diisopropylethylamine (0.910 mL, 5.22 mmole) and HATU (1.186 g, 3.12 mmole) were added and the mixture was stirred at RT for 18 hours. Water (25 mL) was added and the mixture was extracted with DCM (3×10 mL). The combined organics were dried over magnesium sulfate, filtered and concentrated. The residue was dissolved in DCM (5.2 mL) and imidazole (0.194 g, 2.85 mmole) and tert-butyldimethylchlorosilane (0.444 g, 2.95 mmole) were added. The mixture was stirred at RT for 3 hours and water (25 mL) was added. The mixture was extracted with DCM (3×10 mL) and the combined organics were dried over magnesium sulfate, filtered and concentrated. The crude residue was purified by silica gel chromatography to provide methyl N-(2-(4-(((((6-azidohexyl)oxy)carbonyl)amino)methyl)phenyl)thiazole-4-carbonyl)-O-(tert-butyldimethylsilyl)-L-serinate as a pale yellow oil (1.330 g, 82%).

¹H NMR (400 MHz, CDCl₃): δ 8.21 (d, J=8.7 Hz, 1H), 8.09 (s, 1H), 7.98-7.89 (m, 2H), 7.38 (d, J=7.9 Hz, 2H), 5.08 (s, 1H), 4.86 (dt, J=8.7, 3.0 Hz, 1H), 4.43 (d, J=6.1 Hz, 2H), 4.21 (dd, J=10.0, 2.7 Hz, 1H), 4.12 (td, J=6.9, 2.7 Hz, 2H), 3.95 (dd, J=10.1, 3.4 Hz, 1H), 3.79 (s, 3H), 3.27 (t, J=5.8 Hz, 2H), 1.66 (d, J=6.9 Hz, 4H), 1.41 (s, 4H), 0.92 (s, 9H), 0.08 (s, 3H), 0.06 (s, 3H).

### Methyl O-acetyl-N—(N-(2-(4-(((((6-azidohexyl)oxy)carbonyl)amino)methyl)phenyl)thiazole-4-carbonyl)-O-(tert-butyldimethylsilyl)-L-seryl)-L-serinate (50.3)

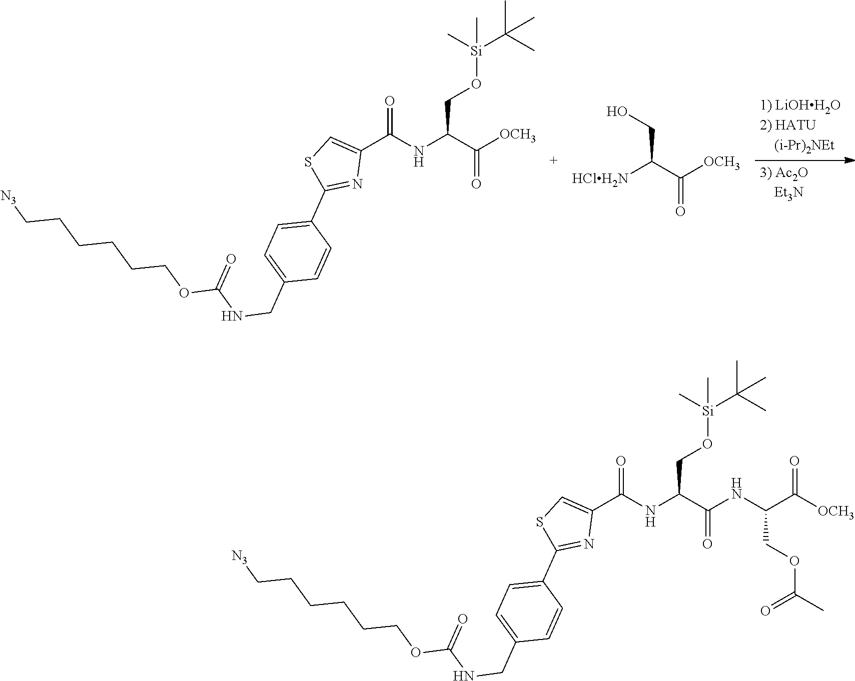

Methyl N-(2-(4-(((((6-azidohexyl)oxy)carbonyl)amino)methyl)phenyl)thiazole-4-carbonyl)-O-(tert-butyldimethylsilyl)-L-serinate (1.330 g, 2.14 mmole) was dissolved in 4/1/1 THF/methanol/water (4.3 mL). Lithium hydroxide monohydrate (0.138 g, 3.29 mmole) was added and the mixture was stirred at RT for 3 hours. Water (25 mL) was added and the mixture was treated with 1N aq. HCl to pH=4. The mixture was extracted with ethyl acetate (3×10 mL) and the combined organics were dried over magnesium sulfate, filtered and concentrated. The residue was dissolved in DCM (4.3 mL) and L-serine methyl ester hydrochloride (0.434 g, 2.79 mmole) was added followed by N,N-diisopropylethylamine (0.750 mL, 4.31 mmole) and HATU (0.980 g, 2.58 mmole). The mixture was stirred at RT for 18 hours and water (25 mL) was added. The mixture was extracted with DCM (3×10 mL) and the combined organics were dried over magnesium sulfate, filtered and concentrated. The residue was dissolved in DCM (4.3 mL) and triethylamine (0.340 mL, 2.43 mmole) and acetic anhydride (0.230 mL, 2.43 mmole) were added. The mixture was stirred at RT for 18 hours and water (25 mL) was added. The mixture was extracted with DCM (3×10 mL) and the combined organics were dried over magnesium sulfate, filtered and concentrated. The crude residue was purified by silica gel chromatography to provide methyl O-acetyl-N—(N-(2-(4-(((((6-azidohexyl)oxy)carbonyl)amino)methyl)phenyl)thiazole-4-carbonyl)-O-(tert-butyldimethylsilyl)-L-seryl)-L-serinate as a pale yellow oil (0.920 g, 57%).

¹H NMR (400 MHz, CDCl₃): δ 8.26 (d, J=7.1 Hz, 1H), 8.11 (d, J=2.6 Hz, 1H), 7.94 (dd, J=8.3, 1.7 Hz, 2H), 7.47 (d, J=7.8 Hz, 1H), 7.37 (d, J=8.0 Hz, 2H), 5.10 (s, 1H), 4.90 (dq, J=6.9, 3.5 Hz, 1H), 4.66 (td, J=7.2, 3.7 Hz, 1H), 4.49 (ddd, J=10.9, 6.9, 4.0 Hz, 1H), 4.45-4.37 (m, 2H), 4.34 (dd, J=11.4, 3.6 Hz, 1H), 4.23 (dt, J=9.8, 3.5 Hz, 1H), 4.10-4.10 (m, 2H), 3.84-3.79 (m, 1H), 3.77 (s, 3H), 3.27 (t, J=7.0 Hz, 2H), 2.02 (s, 3H), 1.67-1.54 (m, 4H), 1.49-1.32 (m, 4H), 0.96 (s, 9H), 0.17 (s, 3H), 0.15 (s, 3H).

### Methyl 2-(2-(2-(4-(((((6-azidohexyl)oxy)carbonyl)amino)methyl)phenyl)thiazole-4-carboxamido)acrylamido)acrylate (50)

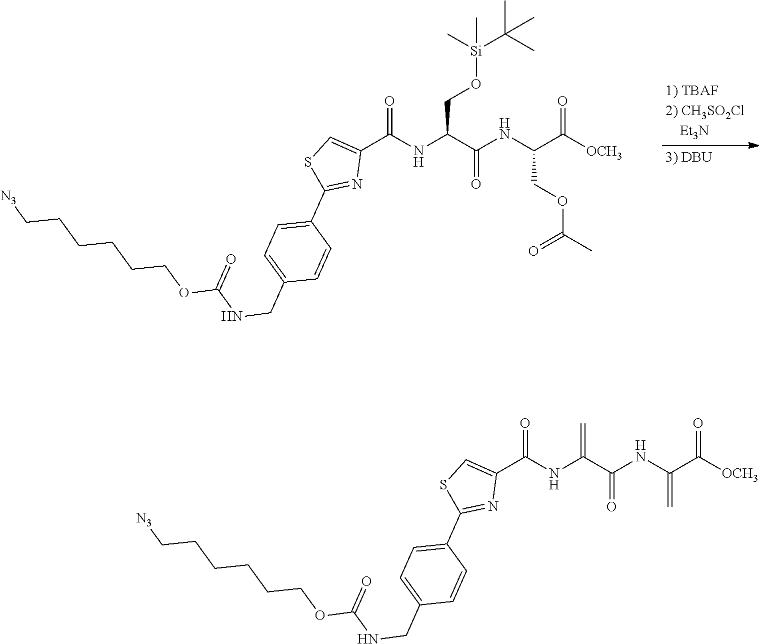 [SMILES: COC(=O)[C@H](COC(C)=O)NC(=O)[C@H](CO[Si](C)(C)C(C)(C)C)NC(=O)c1csc(-c2ccc(CNC(=O)OCCCCCCN=[N+]=[N-])cc2)n1 ; C=C(NC(=O)c1csc(-c2ccc(CNC(=O)OCCCCCCN=[N+]=[N-])cc2)n1)C(=O)NC(=C)C(=O)OC]

Methyl O-acetyl-N—(N-(2-(4-(((((6-azidohexyl)oxy)carbonyl)amino)methyl)phenyl)thiazole-4-carbonyl)-O-(tert-butyldimethylsilyl)-L-seryl)-L-serinate (0.920 g, 1.23 mmole) was dissolved in THF (2.5 mL). TBAF (1M solution in THF, 3.00 mL, 3.00 mmole) was added and the solution was stirred at RT for 2 hours. Water (25 mL) was added and the mixture was extracted with ethyl acetate (3×10 mL). The combined organics were dried over magnesium sulfate, filtered and concentrated. The residue was dissolved in DCM (2.5 mL) and cooled to 0° C. Triethylamine (0.260 mL, 1.45 mmole) and methanesulfonyl chloride (0.145 mL, 1.87 mmole) were added and the mixture was stirred at 0° for 90 minutes. Water (25 mL) was added and the mixture was extracted with DCM (3×10 mL). The combined organics were dried over magnesium sulfate, filtered and concentrated. The residue was dissolved in THF (2.5 mL) and cooled to 0° C. DBU (0.280 mL, 1.87 mmole) was added and the mixture was stirred at 0° for 60 minutes. Water (25 mL) was added and the mixture was extracted with ethyl acetate (3×10 mL). The combined organics were dried over magnesium sulfate, filtered and concentrated. The crude residue was purified by silica gel chromatography to provide methyl 2-(2-(2-(4-(((((6-azidohexyl)oxy)carbonyl)amino)methyl)phenyl)thiazole-4-carboxamido)acrylamido)acrylate as a white solid (0.308 g, 45%).

¹H NMR (400 MHz, CDCl₃): δ 10.01 (s, 1H), 8.55 (s, 1H), 8.13 (s, 1H), 8.03-7.91 (m, 2H), 7.38 (d, J=7.9 Hz, 2H), 6.78 (d, J=2.3 Hz, 1H), 6.71 (s, 1H), 6.03 (d, J=1.3 Hz, 1H), 5.50 (t, J=1.9 Hz, 1H), 5.12 (s, 1H), 4.42 (d, J=6.1 Hz, 2H), 4.11 (td, J=6.9, 1.8 Hz, 2H), 3.90 (s, 3H), 3.27 (t, J=6.8 Hz, 2H), 1.64 (dd, J=14.0, 7.2 Hz, 4H), 1.41 (s, 4H).

### Compound 51: Methyl 2-(2-(2-(4-(2-(tetrahydro-2H-pyran-4-yl)acetamido)piperidin-1-yl)thiazole-4-carboxamido)acrylamido)acrylate

### Methyl O-(tert-butyldimethylsilyl)-N-(2-(4-(2-(tetrahydro-2H-pyran-4-yl)acetamido)piperidin-1-yl)thiazole-4-carbonyl)-L-serinate (51.1)

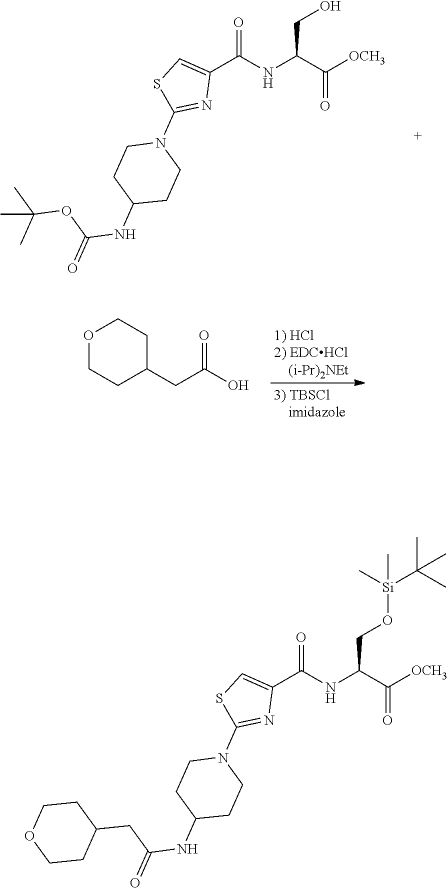 [SMILES: COC(=O)[C@H](CO)NC(=O)c1csc(N2CCC(NC(=O)OC(C)(C)C)CC2)n1 ; O=C(O)CC1CCOCC1 ; COC(=O)[C@H](CO[Si](C)(C)C(C)(C)C)NC(=O)c1csc(N2CCC(NC(=O)CC3CCOCC3)CC2)n1]

Methyl (2-(4-((tert-butoxycarbonyl)amino)piperidin-1-yl)thiazole-4-carbonyl)-L-serinate (Example 46.2, 0.450 g, 1.05 mmole) was dissolved in DCM (2 mL). HCl (4M solution in 1,4-dioxane, 1.00 mL, 4.00 mmole) was added and the mixture was stirred at RT for 3 hours and was concentrated. The residue was suspended in DCM (2 mL) and 2-(tetrahydro-2H-pyran-4-yl)acetic acid (0.227 g, 1.57 mmole) was added. EDC·HCl (0.243 g, 1.27 mmole) and N,N-diisopropylethyl amine (0.370 mL, 2.12 mmole) were added and the mixture was stirred at RT for 18 hours. Water (25 mL) was added and the mixture was extracted with DCM (3×10 mL). The combined organics were dried over magnesium sulfate, filtered and concentrated. The residue was suspended in DCM (4 mL) and imidazole (0.085 g, 1.25 mmole) and tert-butyldimethylchlorosilane (0.181 g, 1.20 mmole) were added. The mixture was stirred at RT for 90 minutes and water (25 mL) was added. The mixture was extracted with DCM (3×10 mL) and the combined organics were dried over magnesium sulfate, filtered and concentrated. The crude residue was purified by silica gel chromatography to provide methyl O-(tert-butyl dimethylsilyl)-N-(2-(4-(2-(tetrahydro-2H-pyran-4-yl)acetamido)piperidin-1-yl)thiazole-4-carbonyl)-L-serinate as a thick, colorless gel (0.470 g, 79%).

¹H NMR (400 MHz, CDCl₃): δ 7.94 (d, J=8.8 Hz, 1H), 7.38 (s, 1H), 5.39 (d, J=7.9 Hz, 1H), 4.78 (ddd, J=8.8, 3.3, 2.5 Hz, 1H), 4.20-4.13 (m, 1H), 4.08-3.98 (m, 2H), 3.98-3.91 (m, 3H), 3.88 (dd, J=10.0, 3.4 Hz, 1H), 3.76 (s, 3H), 3.41 (td, J=11.8, 2.1 Hz, 2H), 3.15 (dtd, J=13.7, 11.4, 2.8 Hz, 2H), 2.09 (dd, J=2.7, 1.5 Hz, 3H), 2.04-1.97 (m, 2H), 1.68-1.61 (m, 2H), 1.50 (dddd, J=21.5, 13.3, 7.0, 2.8 Hz, 2H), 1.39-1.28 (m, 2H), 0.88 (s, 9H), 0.05 (s, 3H), 0.03 (s, 3H).

### Methyl O-acetyl-N—(O-(tert-butyldimethylsilyl)-N-(2-(4-(2-(tetrahydro-2H-pyran-4-yl)acetamido)piperidin-1-yl)thiazole-4-carbonyl)-L-seryl)-L-serinate (51.2)

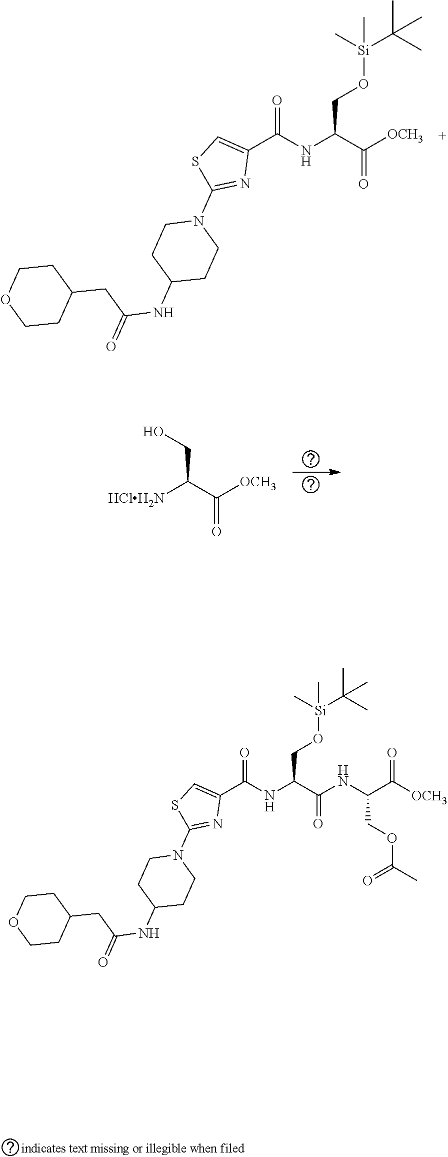

Methyl O-(tert-butyldimethylsilyl)-N-(2-(4-(2-(tetrahydro-2H-pyran-4-yl)acetamido)piperidin-1-yl)thiazole-4-carbonyl)-L-serinate (0.470 g, 0.826 mmole) was dissolved in 4/1/1 THF/methanol/water (1.7 mL). Lithium hydroxide monohydrate (0.070 g, 1.67 mmole) was added and the mixture was stirred at RT for 3 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was dissolved in DCM (1.7 mL). L-Serine methyl ester hydrochloride (0.176 g, 1.13 mmole) was added followed by HATU (0.383 g, 1.01 mmole) and N,N-diisopropylethylamine (0.290 ml, 1.66 mmole). The mixture was stirred at RT for 18 hours and water (25 mL) was added. The mixture was extracted with DCM (3×10 mL) and the combined organics were dried over magnesium sulfate, filtered and concentrated. The residue was dissolved in DCM (1.7 mL). Triethylamine (0.130 mL, 0.928 mmole) and acetic anhydride (0.086 mL, 0.910 mmole) were added and the solution was stirred at RT for 2 hours. Water (25 mL) was added and the mixture was extracted with DCM (3×10 mL). The combined organics were dried over magnesium sulfate, filtered and concentrated. The crude residue was purified by silica gel chromatography to provide methyl O-acetyl-N—(O-(tert-butyldimethylsilyl)-N-(2-(4-(2-(tetrahydro-2H-pyran-4-yl)acetamido)piperidin-1-yl)thiazole-4-carbonyl)-L-seryl)-L-serinate as a colorless gel (0.275 g, 48%).

¹H NMR (400 MHz, CDCl₃): δ 7.97 (d, J=7.1 Hz, 1H), 7.48-7.41 (m, 1H), 7.40 (d, J=2.6 Hz, 1H), 5.41 (d, J=8.0 Hz, 1H), 4.86 (dq, J=8.1, 4.0 Hz, 1H), 4.63-4.55 (m, 1H), 4.51-4.42 (m, 1H), 4.32 (dd, J=11.4, 3.7 Hz, 1H), 4.22-4.16 (m, 1H), 4.07-3.98 (m, 2H), 3.98-3.91 (m, 3H), 3.76 (d, J=2.1 Hz, 3H), 3.75-3.68 (m, 1H), 3.41 (td, J=11.9, 2.1 Hz, 2H), 3.21-3.08 (m, 2H), 2.08 (dd, J=2.6, 1.4 Hz, 3H), 2.02 (d, J=3.4 Hz, 4H), 1.71-1.59 (m, 4H), 1.51 (tq, J=11.6, 6.1, 5.5 Hz, 2H), 1.39-1.29 (m, 2H), 0.92 (d, J=1.0 Hz, 8H), 0.14 (s, 3H), 0.12 (s, 3H).

### Methyl 2-(2-(2-(4-(2-(tetrahydro-2H-pyran-4-yl)acetamido)piperidin-1-yl)thiazole-4-carboxamido)acrylamido)acrylate (51)

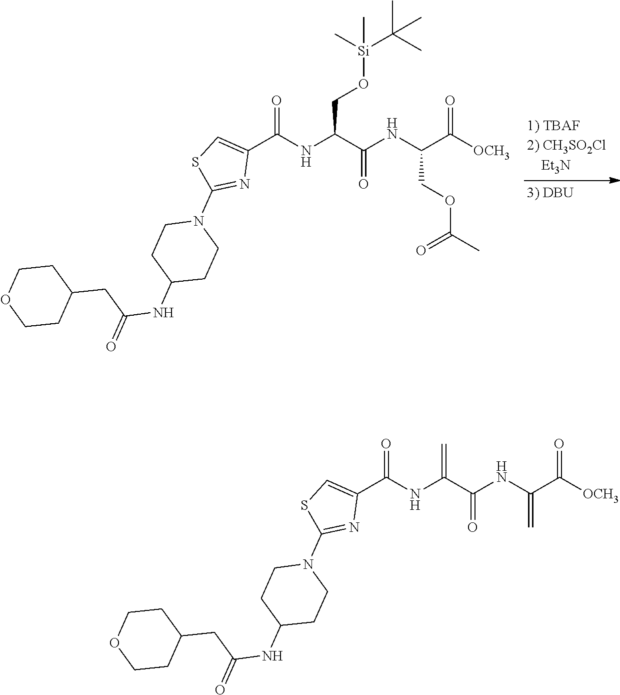 [SMILES: COC(=O)[C@H](COC(C)=O)NC(=O)[C@H](CO[Si](C)(C)C(C)(C)C)NC(=O)c1csc(N2CCC(NC(=O)CC3CCOCC3)CC2)n1 ; C=C(NC(=O)c1csc(N2CCC(NC(=O)CC3CCOCC3)CC2)n1)C(=O)NC(=C)C(=O)OC]

Methyl O-acetyl-N—(O-(tert-butyldimethylsilyl)-N-(2-(4-(2-(tetrahydro-2H-pyran-4-yl)acetamido)piperidin-1-yl)thiazole-4-carbonyl)-L-seryl)-L-serinate (0.275 g, 0.394 mmole) was dissolved in THF (2 mL). TBAF (1M solution in THF, 0.990 mL, 0.990 mmole) was added and the solution was stirred at RT for 18 hours. Water (25 mL) and sat. aq. sodium chloride (15 mL) were added and the mixture was extracted with ethyl acetate (3×10 mL). The combined organics were dried over magnesium sulfate, filtered and concentrated. The residue was dissolved in DCM (2 mL) and cooled to 0° C. Triethylamine (0.084 mL, 0.599 mmole) and methanesulfonyl chloride (0.046 mL, 0.594 mmole) were added and the mixture was stirred at 0° for 2 hours. Water (25 mL) was added and the mixture was extracted with DCM (3×10 mL). The combined organics were dried over magnesium sulfate, filtered and concentrated. The residue was dissolved in THF (2 mL) and cooled to 0° C. DBU (0.090 mL, 0.602 mmole) was added and the mixture was stirred at 0° for 60 minutes. Water (25 mL) and sat. aq. sodium chloride (15 mL) were added and the mixture was extracted with ethyl acetate (3×10 mL). The combined organics were dried over magnesium sulfate, filtered and concentrated. The crude residue was purified by silica gel chromatography to provide methyl 2-(2-(2-(4-(2-(tetrahydro-2H-pyran-4-yl)acetamido)piperidin-1-yl)thiazole-4-carboxamido)acrylamido)acrylate as a white solid (0.067 g, 34%).

¹H NMR (400 MHz, CDCl₃): δ 9.71 (s, 1H), 8.51 (s, 1H), 7.43 (s, 1H), 6.72 (d, J=2.1 Hz, 1H), 6.00 (d, J=1.3 Hz, 1H), 5.44 (t, J=1.9 Hz, 1H), 5.35 (d, J=7.9 Hz, 1H), 4.10-3.92 (m, 5H), 3.89 (s, 3H), 3.41 (td, J=11.8, 2.1 Hz, 2H), 3.25-3.12 (m, 2H), 2.13-2.00 (m, 5H), 1.64 (d, J=13.6 Hz, 2H), 1.55-1.43 (m, 2H), 1.40-1.22 (m, 2H).

### Compound 52: methyl 2-(2-(2-(4-((tetrahydro-2H-pyran-4-carboxamido)methyl)piperidin-1-yl)thiazole-4-carboxamido)acrylamido)acrylate

### Ethyl 2-(4-(((tert-butoxycarbonyl)amino)methyl)piperidin-1-yl)thiazole-4-carboxylate (52.1)

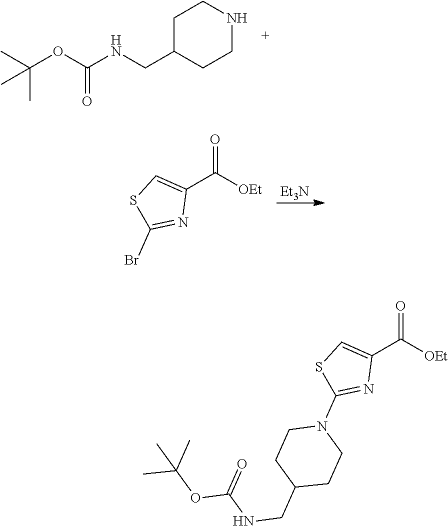 [SMILES: "Et3N" ; CC(C)(C)OC(=O)NCC1CCNCC1 ; CCOC(=O)c1csc(Br)n1 ; CCOC(=O)c1csc(N2CCC(CNC(=O)OC(C)(C)C)CC2)n1]

Tert-butyl (piperidin-4-ylmethyl)carbamate (2.500 g, 11.7 mmole) was dissolved in DMA (12 mL). Ethyl 2-bromothiazole-4-carboxylate (3.027 g, 12.8 mmole) and triethylamine (2.00 mL, 14.3 mmole) were added and the mixture was heated to 80° C. for 20 hours, then was cooled to RT and diluted with ethyl acetate (40 mL). The mixture was washed with water (3×25 mL) and the organics were dried over magnesium sulfate, filtered, and concentrated. The crude residue was purified by silica gel chromatography to provide ethyl 2-(4-(((tert-butoxycarbonyl)amino)methyl)piperidin-1-yl)thiazole-4-carboxylate as a pale yellow oil (4.194 g).

¹H NMR (400 MHz, CDCl₃): δ 7.42 (s, 1H), 4.69 (d, J=6.4 Hz, 1H), 4.35 (q, J=7.1 Hz, 2H), 4.10-4.01 (m, 2H), 3.09-2.94 (m, 4H), 1.86-1.76 (m, 2H), 1.45 (s, 9H), 1.37 (t, J=7.1 Hz, 3H), 1.34-1.28 (m, 2H).

### Methyl (2-(4-(((tert-butoxycarbonyl)amino)methyl)piperidin-1-yl)thiazole-4-carbonyl)-L-serinate (52.2)

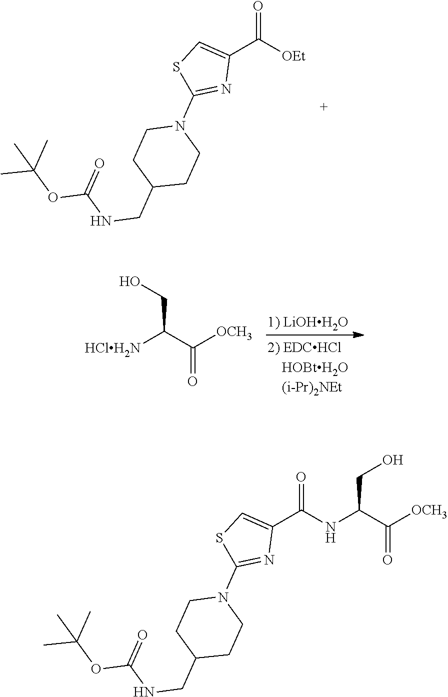

Ethyl 2-(4-(((tert-butoxycarbonyl)amino)methyl)piperidin-1-yl)thiazole-4-carboxylate (4.194 g, 11.4 mmole) was dissolved in 4/1/1 THF/methanol/water (22 mL). Lithium hydroxide monohydrate (0.715 g, 17.0 mmole) was added and the mixture was stirred at RT for 3 hours. Water (50 mL) was added and the solution was treated with 1N aq. HCl to pH=4. The mixture was extracted with ethyl acetate (3×25 mL) and the combined organics were dried over magnesium sulfate, filtered, and concentrated. The residue was dissolved in DCM (22 mL). L-Serine methyl ester hydrochloride (2.128 g, 13.7 mmole) was added followed by N,N-diisopropylethylamine (4.00 mL, 23.0 mmole), HOBt·H₂O (2.106 g, 13.8 mmole), and EDC·HCl (2.617 g, 13.7 mmole). The mixture was stirred at RT for 2½ days and water (50 mL) was added. The two layers were separated and the aqueous layer was extracted with DCM (2×10 mL). The combined organics were dried over magnesium sulfate, filtered, and concentrated and the crude residue was purified by silica gel chromatography to provide methyl (2-(4-(((tert-butoxycarbonyl)amino)methyl)piperidin-1-yl)thiazole-4-carbonyl)-L-serinate as a white solid (5.150 g, 99%).

¹H NMR (400 MHz, CDCl₃): δ 8.01 (d, J=7.4 Hz, 1H), 7.38 (s, 1H), 4.81 (dt, J=7.5, 3.8 Hz, 1H), 4.71 (s, 1H), 4.09-3.97 (m, 4H), 3.83 (s, 3H), 3.12-2.98 (m, 4H), 2.95 (t, J=6.2 Hz, 1H), 1.88-1.78 (m, 2H), 1.47 (s, 9H), 1.40-1.29 (m, 2H).

### Methyl O-(tert-butyldimethylsilyl)-N-(2-(4-((tetrahydro-2H-pyran-4-carboxamido)methyl)piperidin-1-yl)thiazole-4-carbonyl)-L-serinate (52.3)

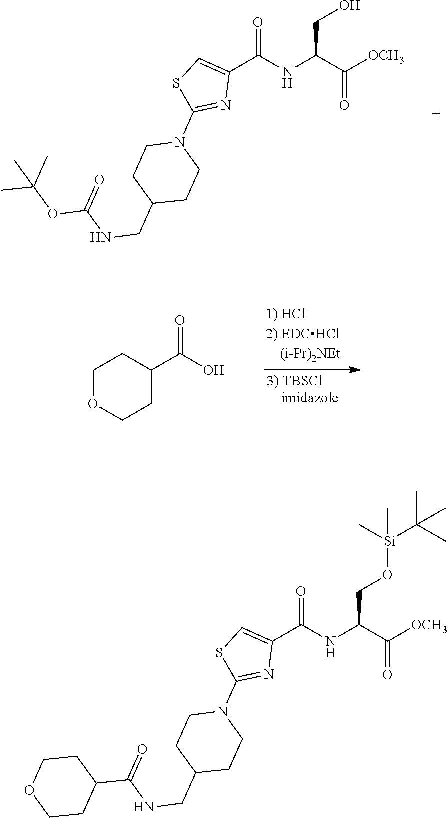 [SMILES: COC(=O)[C@H](CO)NC(=O)c1csc(N2CCC(CNC(=O)OC(C)(C)C)CC2)n1 ; O=C(O)C1CCOCC1 ; COC(=O)[C@H](CO[Si](C)(C)C(C)(C)C)NC(=O)c1csc(N2CCC(CNC(=O)C3CCOCC3)CC2)n1]

Methyl (2-(4-(((tert-butoxycarbonyl)amino)methyl)piperidin-1-yl)thiazole-4-carbonyl)-L-serinate (0.601 g, 1.36 mmole) was dissolved in DCM (2.8 mL). HCl (4M solution in 1,4-dioxane, 1.40 mL, 5.60 mmole) was added and the mixture was stirred at RT for 3 hours, then was concentrated. Tetrahydro-2H-pyran-4-carboxylic acid (0.256 g, 1.97 mmole) was added followed by EDC·HCl (0.333 g, 1.74 mmole) and N,N-diisopropylethylamine (0.500 mL, 2.87 mmole) and the mixture was stirred at RT for 18 hours. Water (25 mL) was added and the mixture was extracted with DCM (3×10 mL). The combined organics were dried over magnesium sulfate, filtered and concentrated. The residue was dissolved in DCM (2.8 mL). Imidazole (0.108 g, 1.59 mmole) and tert-butyldimethylchlorosilane (0.29 g, 1.59 mmole) were added and the solution was stirred at RT for 2 hours. Water (25 mL) was added and the mixture was extracted with DCM (3×10 mL). The combined organics were dried over magnesium sulfate, filtered and concentrated. The crude residue was purified by silica gel chromatography to provide methyl O-(tert-butyldimethylsilyl)-N-(2-(4-((tetrahydro-2H-pyran-4-carboxamido)methyl)piperidin-1-yl)thiazole-4-carbonyl)-L-serinate as a colorless oil (0.570 g, 74%).

¹H NMR (400 MHz, CDCl₃): δ 7.96 (d, J=8.8 Hz, 1H), 7.36 (s, 1H), 5.72 (t, J=6.1 Hz, 1H), 4.82-4.73 (m, 1H), 4.19-4.15 (m, 1H), 4.06-3.93 (m, 4H), 3.88 (dd, J=10.0, 3.4 Hz, 1H), 3.76 (s, 3H), 3.42 (td, J=11.5, 2.9 Hz, 2H), 3.21 (t, J=6.2 Hz, 2H), 3.05-2.91 (m, 2H), 2.36 (tt, J=11.1, 4.4 Hz, 1H), 1.90-1.71 (m, 8H), 1.37-1.28 (m, 1H), 0.88 (s, 9H), 0.05 (s, 3H), 0.03 (s, 3H).

### Methyl O-acetyl-N—(O-(tert-butyldimethylsilyl)-N-(2-(4-((tetrahydro-2H-pyran-4-carboxamido)methyl)piperidin-1-yl)thiazole-4-carbonyl)-L-seryl)-L-serinate (52.4)

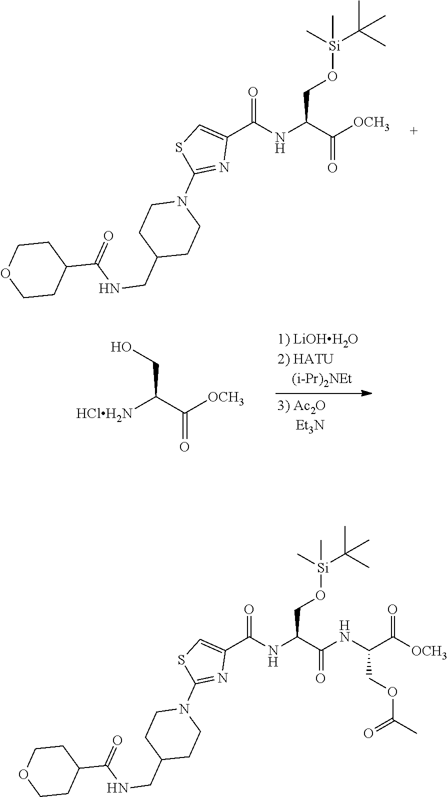

Methyl O-(tert-butyldimethylsilyl)-N-(2-(4-((tetrahydro-2H-pyran-4-carboxamido)methyl)piperidin-1-yl)thiazole-4-carbonyl)-L-serinate (0.570 g, 1.00 mmole) was dissolved in 4/1/1 THF/methanol/water (2 mL). Lithium hydroxide monohydrate (0.082 g, 1.95 mmole) was added and the mixture was stirred at RT for 3 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was dissolved in DCM (2 mL). L-Serine methyl ester hydrochloride (0.205 g, 1.32 mmole) was added followed by HATU (0.474 g, 1.25 mmole) and N,N-diisopropylethylamine (0.350 mL, 2.01 mmole). The mixture was stirred at RT for 18 hours and water (25 mL) was added. The mixture was extracted with DCM (3×10 mL) and the combined organics were dried over magnesium sulfate, filtered and concentrated. The residue was dissolved in DCM (2 mL). Triethylamine (0.160 mL, 1.14 mmole) and acetic anhydride (0.105 mL, 0.952 mmole) were added and the solution was stirred at RT for 2 hours. Water (25 mL) was added and the mixture was extracted with DCM (3×10 mL). The combined organics were dried over magnesium sulfate, filtered and concentrated. The crude residue was purified by silica gel chromatography to provide methyl O-acetyl-N—(O-(tert-butyldimethylsilyl)-N-(2-(4-((tetrahydro-2H-pyran-4-carboxamido)methyl)piperidin-1-yl)thiazole-4-carbonyl)-L-seryl)-L-serinate as a white solid (0.509 g, 73%).

¹H NMR (400 MHz, CDCl₃): δ 7.99 (d, J=7.0 Hz, 1H), 7.44 (d, J=7.8 Hz, 1H), 7.41-7.36 (m, 1H), 5.65 (t, J=6.1 Hz, 1H), 4.87 (dq, J=8.3, 4.1 Hz, 1H), 4.58 (td, J=7.2, 3.7 Hz, 1H), 4.51-4.43 (m, 1H), 4.31 (dd, J=11.4, 3.7 Hz, 1H), 4.22-4.15 (m, 1H), 4.02 (ddt, J=11.6, 8.7, 3.6 Hz, 5H), 3.76 (d, J=2.1 Hz, 4H), 3.74-3.70 (m, 1H), 3.42 (td, J=11.4, 2.9 Hz, 2H), 3.20 (q, J=7.6, 6.9 Hz, 3H), 3.05-2.94 (m, 3H), 2.36 (tt, J=11.0, 4.5 Hz, 1H), 2.02 (d, J=2.7 Hz, 3H), 1.87-1.73 (m, 8H), 1.42 (s, 3H), 1.39-1.28 (m, 2H), 0.92 (s, 9H), 0.14 (s, 3H), 0.12 (s, 3H).

### Methyl 2-(2-(2-(4-((tetrahydro-2H-pyran-4-carboxamido)methyl)piperidin-1-yl)thiazole-4-carboxamido)acrylamido)acrylate (52)

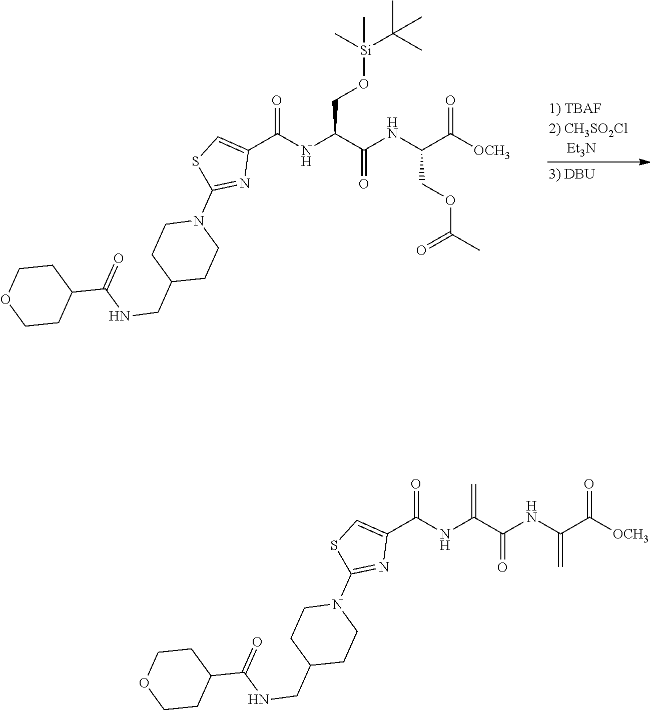 [SMILES: COC(=O)[C@H](COC(C)=O)NC(=O)[C@H](CO[Si](C)(C)C(C)(C)C)NC(=O)c1csc(N2CCC(CNC(=O)C3CCOCC3)CC2)n1 ; C=C(NC(=O)c1csc(N2CCC(CNC(=O)C3CCOCC3)CC2)n1)C(=O)NC(=C)C(=O)OC]

Methyl O-acetyl-N—(O-(tert-butyldimethylsilyl)-N-(2-(4-((tetrahydro-2H-pyran-4-carboxamido)methyl)piperidin-1-yl)thiazole-4-carbonyl)-L-seryl)-L-serinate (0.509 g, 0.729 mmole) was dissolved in THF (2 mL). TBAF (1M solution in THF, 1.80 mL, 1.80 mmole) was added and the solution was stirred at RT for 18 hours. Water (25 mL) and sat. aq. sodium chloride (15 mL) were added and the mixture was extracted with ethyl acetate (3×10 mL). The combined organics were dried over magnesium sulfate, filtered and concentrated. The residue was dissolved in DCM (2 mL) and cooled to 0° C. Triethylamine (0.160 mL, 1.14 mmole) and methanesulfonyl chloride (0.086 mL, 1.11 mmole) were added and the mixture was stirred at 0° for 2 hours. Water (25 mL) was added and the mixture was extracted with DCM (3×10 mL). The combined organics were dried over magnesium sulfate, filtered and concentrated. The residue was dissolved in THF (2 mL) and cooled to 0° C. DBU (0.170 mL, 1.14 mmole) was added and the mixture was stirred at 0° for 60 minutes. Water (25 mL) and sat. aq. sodium chloride (15 mL) were added and the mixture was extracted with ethyl acetate (3×10 mL). The combined organics were dried over magnesium sulfate, filtered and concentrated. The crude residue was purified by silica gel chromatography to provide methyl 2-(2-(2-(4-((tetrahydro-2H-pyran-4-carboxamido)methyl)piperidin-1-yl)thiazole-4-carboxamido)acrylamido)acrylate as a white solid (0.122 g, 33%).

¹H NMR (400 MHz, CDCl₃): δ 9.73 (s, 1H), 8.51 (s, 1H), 7.41 (s, 1H), 6.72 (d, J=2.1 Hz, 1H), 6.65 (s, 1H), 6.00 (d, J=1.3 Hz, 1H), 5.64-5.53 (m, 1H), 5.43 (t, J=1.9 Hz, 1H), 4.10-3.97 (m, 4H), 3.89 (s, 3H), 3.42 (td, J=11.4, 3.0 Hz, 2H), 3.22 (t, J=6.2 Hz, 2H), 3.02 (td, J=12.7, 2.6 Hz, 2H), 2.35 (tt, J=10.9, 4.6 Hz, 1H), 1.89-1.70 (m, 7H), 1.41-1.28 (m, 2H).

### Compound 53: Methyl 2-(2-(2-(4-((tetrahydro-2H-pyran-4-yl)carbamoyl)piperidin-1-yl)thiazole-4-carboxamido)acrylamido)acrylate

### 1-Benzyl 4-(tert-butyl) piperidine-1,4-dicarboxylate (53.1)

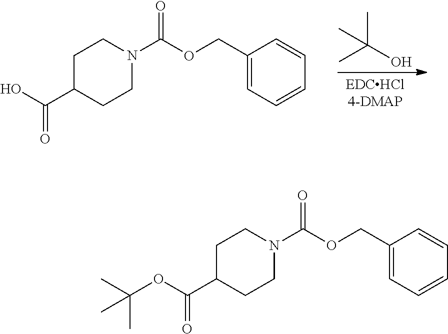

1-((Benzyloxy)carbonyl)piperidine-4-carboxylic acid (3.014 g, 11.4 mmole) was dissolved in DCM (22 mL) and cooled in an ice/salt bath. Tert-butanol (2.80 mL, 29.3 mmole) and 4-DMAP (0.695 g, 5.69 mmole) were added followed by EDC·HCl (2.185 g, 11.4 mmole). The mixture was stirred at 0° C. for 2 hours, then was warmed to RT and stirred for 18 hours. 1N aq. HCl (50 mL) was added and the two layers were separated. The aqueous layer was extracted with DCM (2×15 mL) and the combined organics were dried over magnesium sulfate, filtered and concentrated. The crude residue was purified by silica gel chromatography to provide 1-benzyl 4-(tert-butyl) piperidine-1,4-dicarboxylate as a colorless oil (2.997 g, 82%).

¹H NMR (400 MHz, CDCl₃): δ 7.41-7.29 (m, 5H), 5.12 (s, 2H), 4.11-4.00 (m, 2H), 2.92 (t, J=12.1 Hz, 2H), 2.36 (tt, J=10.8, 3.9 Hz, 1H), 1.86 (d, J=13.4 Hz, 2H), 1.66-1.55 (m, 2H), 1.44 (s, 9H).

### Ethyl 2-(4-(tert-butoxycarbonyl)piperidin-1-yl)thiazole-4-carboxylate (53.2)

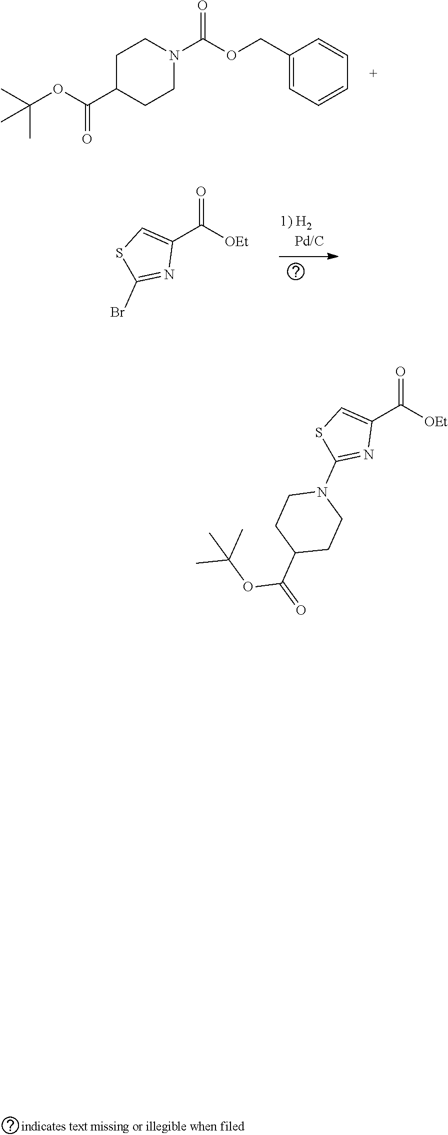 [SMILES: CC(C)(C)OC(=O)C1CCN(C(=O)OCc2ccccc2)CC1 ; CCOC(=O)c1csc(Br)n1 ; CCOC(=O)c1csc(N2CCC(C(=O)OC(C)(C)C)CC2)n1]

A clean, dry flask was charged with 10% palladium on carbon (0.500 g, 0.470 mmole). Enough methanol was added dropwise to wet the catalyst. 1-Benzyl 4-(tert-butyl) piperidine-1,4-dicarboxylate (2.997 g, 9.38 mmole) was dissolved in the remaining methanol (18 mL) and added. The atmosphere was changed to hydrogen (balloon) and the mixture was vigorously stirred at RT for 2 hours. The mixture was filtered through a pad of Celite and the filter cake was washed with methanol. The combined filtrates were concentrated. The residue was dissolved in DMA (10 mL) and ethyl 2-bromothiazole-4-carboxylate (2.224 g, 9.42 mmole) and triethylamine (1.60 mL, 11.4 mmole) were added. The mixture was heated to 80° C. for 18 hours and was cooled to RT. The mixture was diluted with ethyl acetate (50 mL) and washed with water (3×25 mL). The organics were dried over magnesium sulfate, filtered and concentrated. The crude residue was purified by silica gel chromatography to provide ethyl 2-(4-(tert-butoxycarbonyl)piperidin-1-yl)thiazole-4-carboxylate as a yellow solid (2.340 g, 73%).

¹H NMR (400 MHz, CDCl₃): δ 7.44 (s, 1H), 4.35 (q, J=7.0 Hz, 2H), 3.97 (dt, J=13.0, 3.7 Hz, 2H), 3.21-3.07 (m, 2H), 2.43 (tt, J=10.7, 3.0 Hz, 1H), 2.03-1.92 (m, 2H), 1.87-1.72 (m, 2H), 1.46 (s, 9H), 1.37 (t, J=7.1 Hz, 3H).

### Tert-butyl (S)-1-(4-((3-hydroxy-1-methoxy-1-oxopropan-2-yl)carbamoyl)thiazol-2-yl)piperidine-4-carboxylate (53.3)

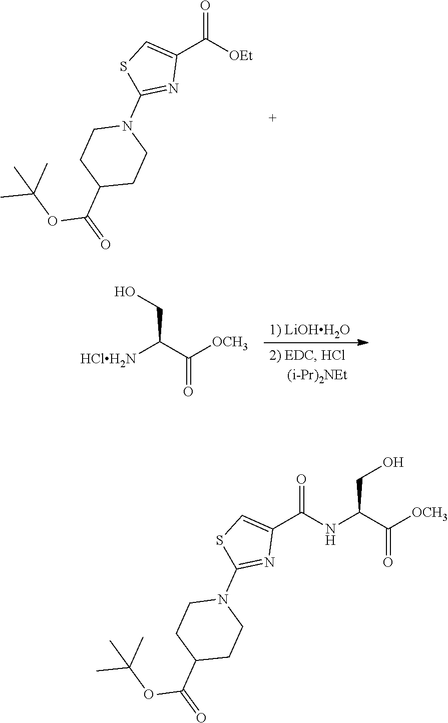

Ethyl 2-(4-(tert-butoxycarbonyl)piperidin-1-yl)thiazole-4-carboxylate (2.340 g, 6.87 mmole) was dissolved in 4/1/1 THF/methanol/water (14 mL). Lithium hydroxide monohydrate (434 g, 10.3 mmole) was added and the mixture was stirred at RT for 90 minutes. Water (25 mL) was added and the solution was treated with 1N aq. HCl to pH=4. The mixture was extracted with ethyl acetate (3×15 mL) and the combined organics were dried over magnesium sulfate, filtered and concentrated. The residue was dissolved in DCM (14 mL) and L-serine methyl ester hydrochloride (1.429 g, 9.18 mmole) was added. N,N-Diisopropylethylamine (2.40 mL, 13.8 mmole) and EDC·HCl (1.586, 8.27 mmole) were added and the mixture was stirred at RT for 18 hours. Water (25 mL) and 1N aq. HCl (25 mL) were added and the two layers were separated. The aqueous layer was extracted with DCM (2×15 mL) and the combined organics were dried over magnesium sulfate, filtered and concentrated. The crude residue was purified by silica gel chromatography to provide tert-butyl (S)-1-(4-((3-hydroxy-1-methoxy-1-oxopropan-2-yl)carbamoyl)thiazol-2-yl)piperidine-4-carboxylate as a white solid (1.783 g, 63%).

¹H NMR (400 MHz, CDCl₃): δ 8.00 (d, J=7.4 Hz, 1H), 7.38 (s, 1H), 4.79 (dt, J=7.6, 3.9 Hz, 1H), 4.04 (d, J=3.9 Hz, 2H), 3.97-3.87 (m, 2H), 3.81 (s, 3H), 3.13 (ddd, J=13.1, 11.1, 3.2 Hz, 2H), 3.00 (br s, 1H), 2.44 (tt, J=10.6, 3.8 Hz, 1H), 2.03-1.95 (m, 2H), 1.87-1.72 (m, 2H), 1.46 (s, 9H).

### Methyl O-(tert-butyldimethylsilyl)-N-(2-(4-((tetrahydro-2H-pyran-4-yl)carbamoyl)piperidin-1-yl)thiazole-4-carbonyl)-L-serinate (53.4)

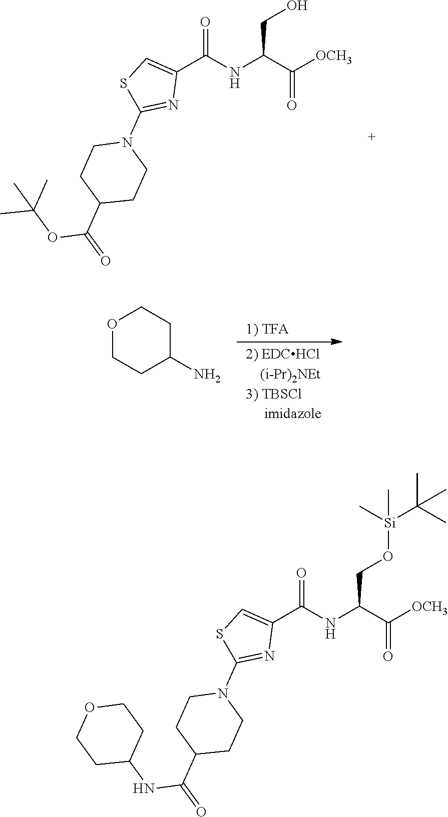 [SMILES: COC(=O)[C@H](CO)NC(=O)c1csc(N2CCC(C(=O)OC(C)(C)C)CC2)n1 ; NC1CCOCC1 ; COC(=O)[C@H](CO[Si](C)(C)C(C)(C)C)NC(=O)c1csc(N2CCC(C(=O)NC3CCOCC3)CC2)n1]

Tert-butyl (S)-1-(4-((3-hydroxy-1-methoxy-1-oxopropan-2-yl)carbamoyl)thiazol-2-yl)piperidine-4-carboxylate (0.753 g, 1.82 mmole) was dissolved in DCM (4 mL). Trifluoroacetic acid (2.00 mL, 26.0 mmole) was added and the mixture was stirred at RT for 24 hours, then was concentrated. The residue was suspended in toluene (5 mL) and concentrated. DCM (4 mL) and N,N-diisopropyl ethyl amine (0.640 mL, 3.67 mmole) were added. Tetrahydro-2H-pyran-4-amine (0.250 mL, 2.41 mmole) and EDC·HCl (0.421 g, 2.20 mmole) were added and the mixture was stirred at RT for 18 hours and water (25 mL) was added. The mixture was extracted with DCM (3×10 mL) and the combined organics were dried over magnesium sulfate, filtered and concentrated. The residue was dissolved in DCM (4 mL) and imidazole (0.139 g, 2.04 mmole) and tert-butyldimethylchlorosilane (0.304 g, 2.02 mmole) were added. The mixture was stirred at RT for 18 hours and water (25 mL) was added. The mixture was extracted with DCM (3×10 mL) and the combined organics were dried over magnesium sulfate, filtered and concentrated. The crude residue was purified by silica gel chromatography to provide methyl O-(tert-butyldimethylsilyl)-N-(2-(4-((tetrahydro-2H-pyran-4-yl)carbamoyl)piperidin-1-yl)thiazole-4-carbonyl)-L-serinate as a thick, pale yellow oil (0.300 g, 30%).

¹H NMR (400 MHz, CDCl₃): δ 7.96 (d, J=8.8 Hz, 1H), 7.38 (s, 1H), 5.39 (d, J=7.9 Hz, 1H), 4.83-4.74 (m, 1H), 4.16 (dd, J=10.0, 2.5 Hz, 1H), 4.08-3.92 (m, 5H), 3.92-3.85 (m, 1H), 3.76 (s, 3H), 3.48 (td, J=11.8, 2.1 Hz, 2H), 3.05 (dddd, J=12.8, 11.4, 4.9, 3.4 Hz, 2H), 2.29 (tt, J=11.3, 3.9 Hz, 1H), 1.97-1.79 (m, 6H), 1.46 (dtd, J=12.9, 11.4, 4.5 Hz, 2H), 0.88 (s, 9H), 0.05 (s, 3H), 0.03 (s, 3H).

### Methyl O-acetyl-N—(O-(tert-butyldimethylsilyl)-N-(2-(4-((tetrahydro-2H-pyran-4-yl)carbamoyl)piperidin-1-yl)thiazole-4-carbonyl)-L-seryl)-L-serinate (53.5)

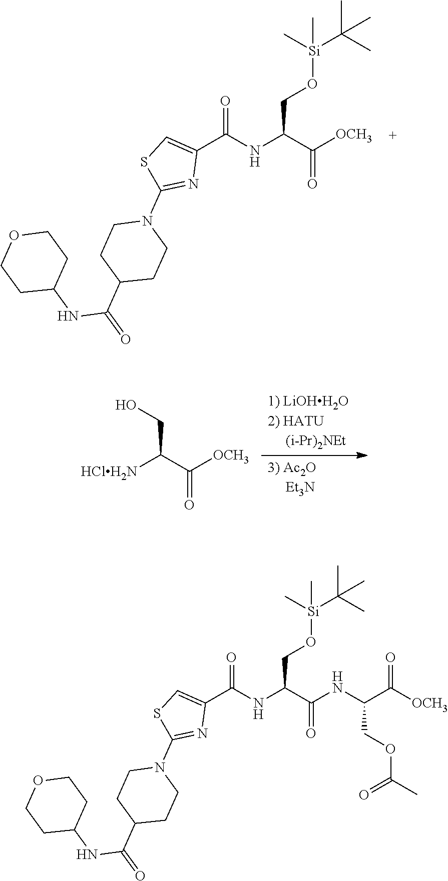

Methyl O-(tert-butyldimethylsilyl)-N-(2-(4-((tetrahydro-2H-pyran-4-yl)carbamoyl)piperidin-1-yl)thiazole-4-carbonyl)-L-serinate (0.300 g, 0.541 mmole) was dissolved in 4/1/1 THF/methanol/water (2 mL). Lithium hydroxide monohydrate (0.045 g, 1.07 mmole) was added and the mixture was stirred at RT for 3 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was dissolved in DCM (2 mL). L-Serine methyl ester hydrochloride (0.117 g, 0.752 mmole) was added followed by HATU (0.245 g, 0.644 mmole) and N,N-diisopropylethylamine (0.190 mL, 1.09 mmole). The mixture was stirred at RT for 18 hours and water (25 mL) was added. The mixture was extracted with DCM (3×10 mL) and the combined organics were dried over magnesium sulfate, filtered and concentrated. The residue was dissolved in DCM (2 mL). Triethylamine (0.084 mL, 0.599 mmole) and acetic anhydride (0.057 mL, 0.603 mmole) were added and the solution was stirred at RT for 18 hours. Water (25 mL) was added and the mixture was extracted with DCM (3×10 mL). The combined organics were dried over magnesium sulfate, filtered and concentrated. The crude residue was purified by silica gel chromatography to provide methyl O-acetyl-N—(O-(tert-butyldimethylsilyl)-N-(2-(4-((tetrahydro-2H-pyran-4-yl)carbamoyl)piperidin-1-yl)thiazole-4-carbonyl)-L-seryl)-L-serinate as a thick, colorless gel (0.227 g, 61%).

¹H NMR (400 MHz, CDCl₃): δ 7.96 (d, J=7.1 Hz, 1H), 7.45-7.39 (m, 1H), 7.37 (d, J=2.7 Hz, 1H), 5.34 (d, J=7.9 Hz, 1H), 4.84 (dq, J=8.4, 4.2 Hz, 1H), 4.56 (ddd, J=7.3, 6.1, 3.6 Hz, 1H), 4.50-4.39 (m, 1H), 4.29 (dd, J=11.4, 3.7 Hz, 1H), 4.20-4.12 (m, 1H), 4.07-3.89 (m, 5H), 3.74 (d, J=2.5 Hz, 3H), 3.72-3.65 (m, 1H), 3.46 (td, J=11.8, 2.2 Hz, 2H), 3.08-2.98 (m, 2H), 2.26 (tt, J=11.2, 3.9 Hz, 1H), 2.00 (s, 3H), 1.95-1.75 (m, 6H), 1.51-1.36 (m, 2H), 0.90 (s, 9H), 0.12 (s, 3H), 0.11 (s, 3H).

### Methyl 2-(2-(2-(4-((tetrahydro-2H-pyran-4-yl)carbamoyl)piperidin-1-yl)thiazole-4-carboxamido)acrylamido)acrylate (53)

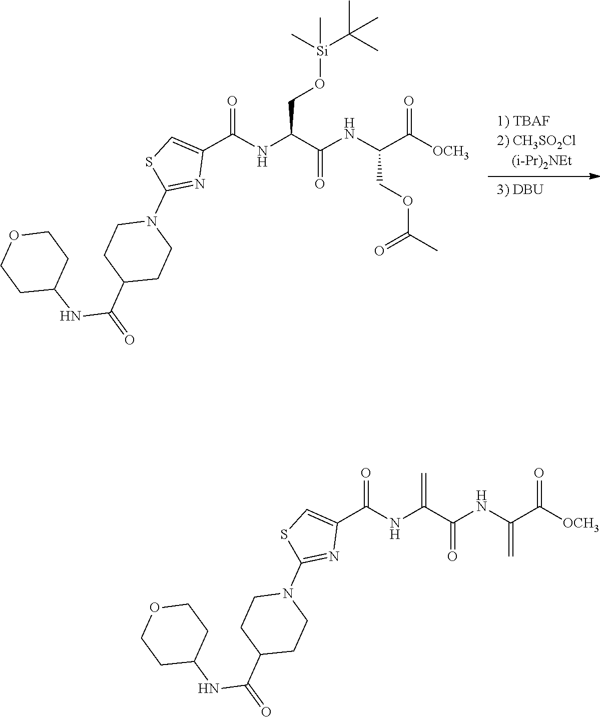 [SMILES: COC(=O)[C@H](COC(C)=O)NC(=O)[C@H](CO[Si](C)(C)C(C)(C)C)NC(=O)c1csc(N2CCC(C(=O)NC3CCOCC3)CC2)n1 ; C=C(NC(=O)c1csc(N2CCC(C(=O)NC3CCOCC3)CC2)n1)C(=O)NC(=C)C(=O)OC]

Methyl O-acetyl-N—(O-(tert-butyldimethylsilyl)-N-(2-(4-((tetrahydro-2H-pyran-4-yl)carbamoyl)piperidin-1-yl)thiazole-4-carbonyl)-L-seryl)-L-serinate (0.227 g, 0.332 mmole) was dissolved in THF (2 mL). TBAF (1M solution in THF, 0.830 mL, 0.830 mmole) was added and the solution was stirred at RT for 18 hours. Water (25 mL) was added and the mixture was extracted with ethyl acetate (3×10 mL). The combined organics were dried over magnesium sulfate, filtered and concentrated. The residue was dissolved in DCM (2 mL) and cooled to 0° C. Triethylamine (0.070 mL, 0.499 mmole) and methanesulfonyl chloride (0.039 mL, 0.504 mmole) were added and the mixture was stirred at 0° for 90 minutes. Water (25 mL) was added and the mixture was extracted with DCM (3×10 mL). The combined organics were dried over magnesium sulfate, filtered and concentrated. The residue was dissolved in THF (2 mL) and cooled to 0° C. DBU (0.080 mL, 0.535 mmole) was added and the mixture was stirred at 0° for 90 minutes. Water (25 mL) was added and the mixture was extracted with ethyl acetate (3×10 mL). The combined organics were dried over magnesium sulfate, filtered and concentrated. The crude residue was purified by silica gel chromatography to provide methyl 2-(2-(2-(4-((tetrahydro-2H-pyran-4-yl)carbamoyl)piperidin-1-yl)thiazole-4-carboxamido)acrylamido)acrylate as a white solid (0.074 g, 45%).

¹H NMR (400 MHz, CDCl₃): δ 9.72 (s, 1H), 8.51 (s, 1H), 7.43 (s, 1H), 6.72 (d, J=2.1 Hz, 1H), 6.65 (s, 1H), 6.00 (d, J=1.3 Hz, 1H), 5.44 (t, J=1.9 Hz, 1H), 5.40 (d, J=8.0 Hz, 1H), 4.10-4.04 (m, 2H), 4.04-3.92 (m, 3H), 3.89 (s, 3H), 3.48 (td, J=11.8, 2.1 Hz, 2H), 3.08 (ddd, J=13.0, 11.6, 3.2 Hz, 2H), 2.29 (tt, J=11.3, 3.8 Hz, 1H), 2.00-1.78 (m, 6H), 1.46 (dtd, J=12.9, 11.4, 4.5 Hz, 2H).

### Compound 54: methyl (S)-2-(2-(2-(3-(tetrahydro-2H-pyran-4-carboxamido)piperidin-1-yl)thiazole-4-carboxamido)acrylamido)acrylate

### Ethyl (S)-2-(3-((tert-butoxycarbonyl)amino)piperidin-1-yl)thiazole-4-carboxylate (54.1)

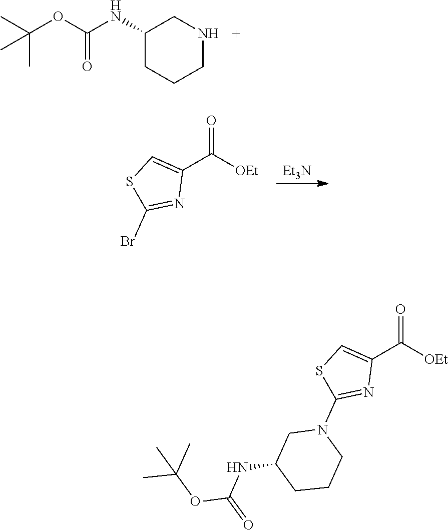 [SMILES: "Et3N" ; CC(C)(C)OC(=O)N[C@H]1CCCNC1 ; CCOC(=O)c1csc(Br)n1 ; CCOC(=O)c1csc(N2CCC[C@H](NC(=O)OC(C)(C)C)C2)n1]

Tert-butyl (S)-piperidin-3-ylcarbamate (2.033 g, 10.2 mmole) was dissolved in DMA (10 mL). Ethyl 2-bromothiazole-4-carboxylate (2.398 g, 10.2 mmole) and triethylamine (1.70 mL, 12.1 mmole) were added and the mixture was heated to 80° C. for 18 hours. The mixture was cooled to RT and diluted with ethyl acetate (50 mL). The mixture was washed with water (3×25 mL) and the organics were dried over magnesium sulfate, filtered and concentrated. The crude residue was purified by silica gel chromatography to provide ethyl (S)-2-(3-((tert-butoxycarbonyl)amino)piperidin-1-yl)thiazole-4-carboxylate as an orange solid (3.087 g, 85%).

¹H NMR (400 MHz, CDCl₃): δ 7.44 (s, 1H), 4.70 (s, 1H), 4.35 (q, J=7.1 Hz, 2H), 3.80 (s, 1H), 3.70 (d, J=12.5 Hz, 1H), 3.61 (s, 1H), 3.46 (s, 1H), 3.29 (t, J=9.9 Hz, 1H), 1.90 (qd, J=7.5, 4.0 Hz, 1H), 1.82 (dtt, J=10.9, 7.3, 3.5 Hz, 1H), 1.75-1.64 (m, 1H), 1.60 (s, 1H), 1.45 (s, 9H), 1.37 (t, J=7.1 Hz, 3H).

### Methyl (2-((S)-3-((tert-butoxycarbonyl)amino)piperidin-1-yl)thiazole-4-carbonyl)-L-serinate (54.2)

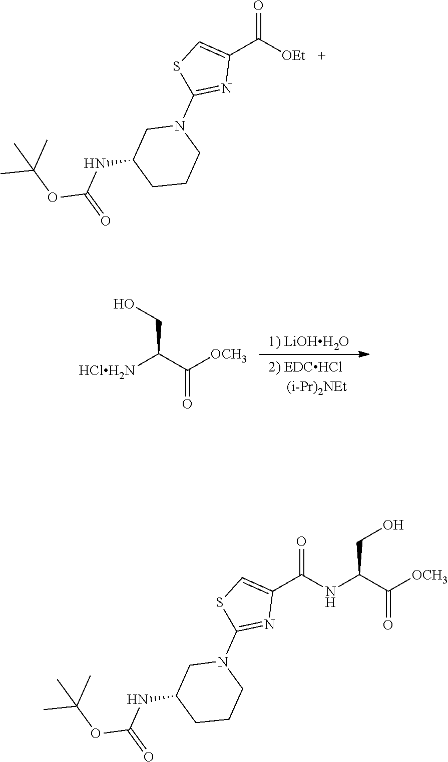

Ethyl (S)-2-(3-((tert-butoxycarbonyl)amino)piperidin-1-yl)thiazole-4-carboxylate (3.087 g, 8.68 mmole) was dissolved in 4/1/1 THF/methanol/water (15 mL). Lithium hydroxide monohydrate (0.545 g, 13.0 mmole) was added and the mixture was stirred at RT for 2 hours. Water (75 mL) was added and the solution was treated with 1N aq. HCl to pH=4. The mixture was extracted with ethyl acetate (3×25 mL) and the combined organics were dried over magnesium sulfate, filtered and concentrated. The residue was suspended in DCM (15 mL) and L-serine methyl ester hydrochloride (1.762 g, 11.3 mmole) was added. N,N-Diisopropylethylamine (3.00 mL, 17.2 mmole) and EDC·HCl (2.013 g, 10.5 mmole) were added and the mixture was stirred at RT for 18 hours. Water (50 mL) was added and the two layers were separated. The aqueous layer was extracted with DCM (2×15 mL) and the combined organics were dried over magnesium sulfate, filtered and concentrated. The crude residue was purified by silica gel chromatography to provide methyl (2-((S)-3-((tert-butoxycarbonyl)amino)piperidin-1-yl)thiazole-4-carbonyl)-L-serinate as a white solid (1.619 g, 44%).

¹H NMR (400 MHz, CDCl₃): δ 7.99 (d, J=7.5 Hz, 1H), 7.40 (s, 1H), 4.85-4.66 (m, 2H), 4.05 (dt, J=7.2, 3.8 Hz, 2H), 3.82 (s, 4H), 3.74 (dd, J=12.8, 3.5 Hz, 1H), 3.56 (d, J=13.0 Hz, 1H), 3.41 (s, 1H), 3.25 (d, J=9.9 Hz, 1H), 2.96 (s, 1H), 1.92 (ddt, J=11.8, 7.5, 3.4 Hz, 1H), 1.83 (dh, J=10.5, 3.4 Hz, 1H), 1.60 (dtd, J=11.9, 8.0, 3.5 Hz, 1H), 1.45 (s, 9H).

### Methyl O-(tert-butyldimethylsilyl)-N-(2-((S)-3-(tetrahydro-2H-pyran-4-carboxamido)piperidin-1-yl)thiazole-4-carbonyl)-L-serinate (54.3)

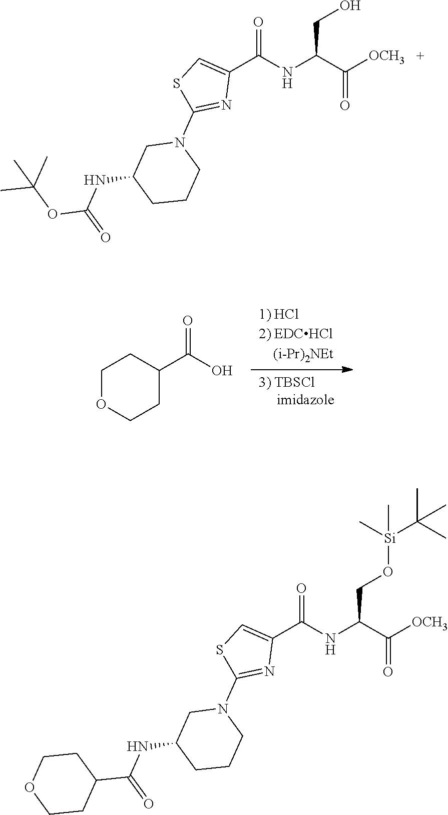 [SMILES: COC(=O)[C@H](CO)NC(=O)c1csc(N2CCC[C@H](NC(=O)OC(C)(C)C)C2)n1 ; O=C(O)C1CCOCC1 ; COC(=O)[C@H](CO[Si](C)(C)C(C)(C)C)NC(=O)c1csc(N2CCC[C@H](NC(=O)C3CCOCC3)C2)n1]

Methyl (2-((S)-3-((tert-butoxycarbonyl)amino)piperidin-1-yl)thiazole-4-carbonyl)-L-serinate (0.505 g, 1.18 mmole) was dissolved in DCM (4.6 mL). HCl (4M solution in 1,4-dioxane, 1.20 mL, 4.80 mmole) was added and the mixture was stirred at RT for 4 hours, then was concentrated. The residue was suspended in DCM (2.3 mL) and tetrahydro-2H-pyran-4-carboxylic acid (0.202 g, 1.55 mmole) was added. EDC·HCl (0.279 g, 1.46 mmole) and N,N-diisopropylethylamine (0.300 mL, 1.72 mmole) were added and the mixture was stirred at RT for 4 days. Water (25 mL) was added and the mixture was extracted with DCM (3×10 mL). The combined organics were dried over magnesium sulfate, filtered and concentrated. The residue was dissolved in DCM (2.3 mL). Imidazole (0094 g, 1.38 mmole) and tert-butyldimethylchlorosilane (0.192 g, 1.27 mmole) were added and the mixture was stirred at RT for 18 hours. Water (25 mL) was added and the mixture was extracted with DCM (3×10 mL). The combined organics were dried over magnesium sulfate, filtered and concentrated. The crude residue was purified by silica gel chromatography to provide methyl O-(tert-butyldimethylsilyl)-N-(2-((S)-3-(tetrahydro-2H-pyran-4-carboxamido)piperidin-1-yl)thiazole-4-carbonyl)-L-serinate as a thick, colorless oil (0.301 g, 46%).

¹H NMR (400 MHz, CDCl₃): δ 7.92 (d, J=8.8 Hz, 1H), 7.40 (s, 1H), 5.71 (d, J=7.7 Hz, 1H), 4.79 (ddd, J=8.8, 3.4, 2.6 Hz, 1H), 4.16 (dd, J=10.0, 2.6 Hz, 2H), 4.00 (ddt, J=11.7, 3.9, 1.5 Hz, 2H), 3.89 (dd, J=10.0, 3.4 Hz, 1H), 3.77 (s, 3H), 3.67-3.59 (m, 1H), 3.55 (dd, J=12.8, 3.4 Hz, 1H), 3.48-3.35 (m, 4H), 2.32 (tt, J=11.2, 4.3 Hz, 1H), 1.88-1.67 (m, 8H), 0.88 (s, 9H), 0.05 (s, 3H), 0.03 (s, 3H).

### Methyl O-acetyl-N—(O-(tert-butyldimethylsilyl)-N-(2-((S)-3-(tetrahydro-2H-pyran-4-carboxamido)piperidin-1-yl)thiazole-4-carbonyl)-L-seryl)-L-serinate (54.4)

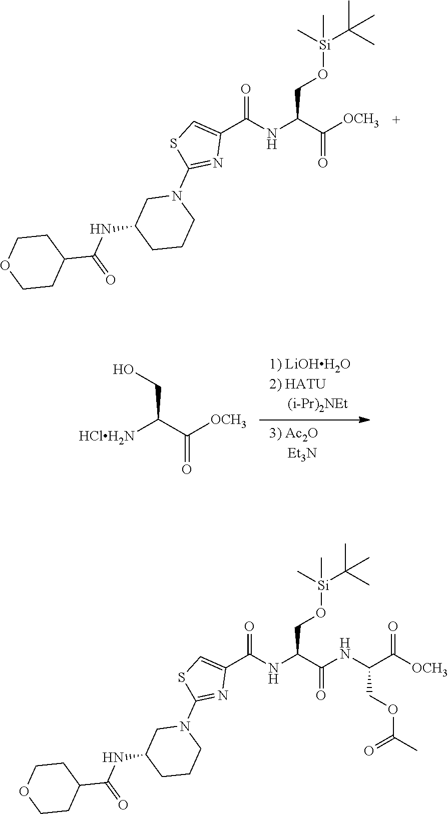

Methyl O-(tert-butyldimethylsilyl)-N-(2-((S)-3-(tetrahydro-2H-pyran-4-carboxamido)piperidin-1-yl)thiazole-4-carbonyl)-L-serinate (0.301 g, 0.543 mmole) was dissolved in 4/1/1 THF/methanol/water (2 mL). Lithium hydroxide monohydrate (0.054 g, 1.29 mmole) was added and the mixture was stirred at RT for 3 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was dissolved in DCM (2 mL). L-Serine methyl ester hydrochloride (0.114 g, 0.733 mmole) was added followed by HATU (0.248 g, 0.652 mmole) and N,N-diisopropylethylamine (0.190 mL, 1.09 mmole). The mixture was stirred at RT for 18 hours and water (25 mL) was added. The mixture was extracted with DCM (3×10 mL) and the combined organics were dried over magnesium sulfate, filtered and concentrated. The residue was dissolved in DCM (2 mL). Triethylamine (0.084 mL, 0.599 mmole) and acetic anhydride (0.057 mL, 0.603 mmole) were added and the solution was stirred at RT for 3 hours. Water (25 mL) was added and the mixture was extracted with DCM (3×10 mL). The combined organics were dried over magnesium sulfate, filtered and concentrated. The crude residue was purified by silica gel chromatography to provide methyl O-acetyl-N—(O-(tert-butyldimethylsilyl)-N-(2-((S)-3-(tetrahydro-2H-pyran-4-carboxamido)piperidin-1-yl)thiazole-4-carbonyl)-L-seryl)-L-serinate as a thick, colorless oil (0.182 g, 49%).

¹H NMR (400 MHz, CDCl₃): δ 7.92 (d, J=7.2 Hz, 1H), 7.43 (d, J=7.7 Hz, 1H), 7.39 (s, 1H), 5.68 (t, J=7.7 Hz, 1H), 4.84 (dq, J=7.2, 3.6 Hz, 1H), 4.56 (td, J=7.3, 3.8 Hz, 1H), 4.45 (ddd, J=10.6, 6.7, 3.9 Hz, 1H), 4.30 (dd, J=11.4, 3.7 Hz, 1H), 4.19-4.07 (m, 2H), 4.03-3.92 (m, 2H), 3.74 (d, J=3.1 Hz, 3H), 3.73-3.67 (m, 1H), 3.56 (ddd, J=19.7, 11.1, 4.1 Hz, 2H), 3.48-3.31 (m, 4H), 2.29 (tt, J=11.1, 4.5 Hz, 1H), 2.01 (d, J=2.7 Hz, 3H), 1.86-1.65 (m, 8H), 0.90 (s, 9H), 0.12 (s, 3H), 0.10 (s, 3H).

### Methyl (S)-2-(2-(2-(3-(tetrahydro-2H-pyran-4-carboxamido)piperidin-1-yl)thiazole-4-carboxamido)acrylamido)acrylate (54)

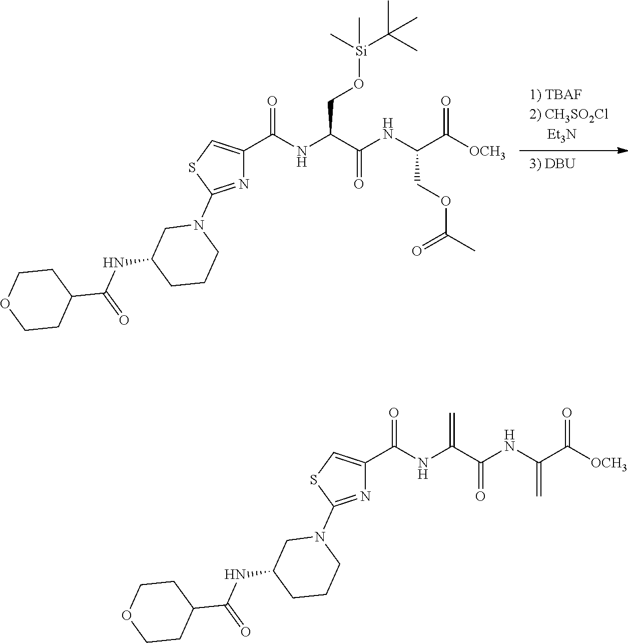 [SMILES: COC(=O)[C@H](COC(C)=O)NC(=O)[C@H](CO[Si](C)(C)C(C)(C)C)NC(=O)c1csc(N2CCC[C@H](NC(=O)C3CCOCC3)C2)n1 ; C=C(NC(=O)c1csc(N2CCC[C@H](NC(=O)C3CCOCC3)C2)n1)C(=O)NC(=C)C(=O)OC]

Methyl O-acetyl-N—(O-(tert-butyldimethylsilyl)-N-(2-((S)-3-(tetrahydro-2H-pyran-4-carboxamido)piperidin-1-yl)thiazole-4-carbonyl)-L-seryl)-L-serinate (0.182 g, 0.266 mmole) was dissolved in THF (2 mL). TBAF (1M solution in THF, 0.670 mL, 0.670 mmole) was added and the solution was stirred at RT for 18 hours. Water (25 mL) was added and the mixture was extracted with ethyl acetate (3×10 mL). The combined organics were dried over magnesium sulfate, filtered and concentrated. The residue was dissolved in DCM (2 mL) and cooled to 0° C. Triethylamine (0.056 mL, 0.400 mmole) and methanesulfonyl chloride (0.031 mL, 0.401 mmole) were added and the mixture was stirred at 0° for 90 minutes. Water (25 mL) was added and the mixture was extracted with DCM (3×10 mL). The combined organics were dried over magnesium sulfate, filtered and concentrated. The residue was dissolved in THF (2 mL) and cooled to 0° C. DBU (0.060 mL, 0.401 mmole) was added and the mixture was stirred at 0° for 90 minutes. Water (25 mL) was added and the precipitated solid was collected by filtration to provide methyl (S)-2-(2-(2-(3-(tetrahydro-2H-pyran-4-carboxamido)piperidin-1-yl)thiazole-4-carboxamido)acrylamido)acrylate as a white solid (0.045 g, 34%).

¹H NMR (400 MHz, CDCl₃): δ 9.69 (s, 1H), 8.51 (s, 1H), 7.45 (s, 1H), 6.72 (d, J=2.1 Hz, 1H), 6.65 (s, 1H), 6.00 (d, J=1.2 Hz, 1H), 5.78 (d, J=7.5 Hz, 1H), 5.44 (t, J=1.9 Hz, 1H), 4.17 (s, 1H), 4.04-3.94 (m, 2H), 3.89 (s, 3H), 3.67-3.53 (m, 3H), 3.53-3.43 (m, 1H), 3.38 (tt, J=11.4, 3.3 Hz, 2H), 2.32 (tt, J=11.1, 4.4 Hz, 1H), 1.91-1.66 (m, 8H).

### Compound 55. Methyl 2-(2-(2-(4-(2-methoxyacetamido)piperidin-1-yl)thiazole-4-carboxamido)acrylamido)acrylate

### Methyl O-(tert-butyldimethylsilyl)-N-(2-(4-(2-methoxyacetamido)piperidin-1-yl)thiazole-4-carbonyl)-L-serinate (55.1)

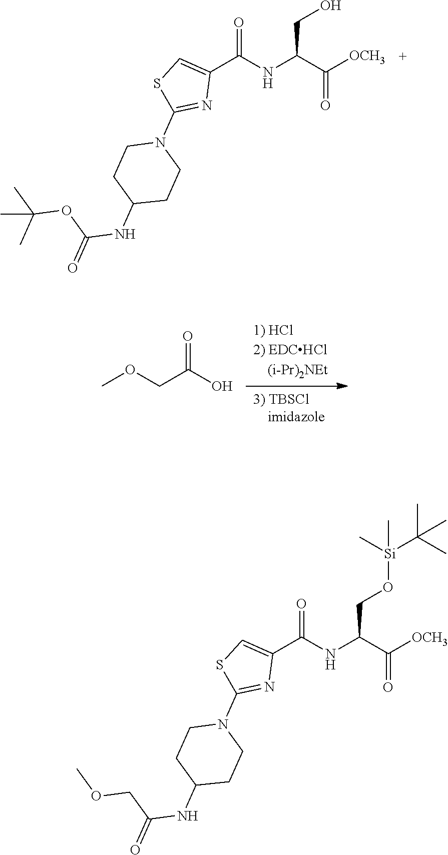

Methyl (2-(4-((tert-butoxycarbonyl)amino)piperidin-1-yl)thiazole-4-carbonyl)-L-serinate (Example 46.2, 0.449 g, 1.05 mmole) was dissolved in DCM (4.2 mL). HCl (4M solution in 1,4-dioxane, 1.05 mL, 4.20 mmole) was added and the mixture was stirred at RT for 4 hours, then was concentrated. The residue was suspended in DCM (2.1 mL) and 2-methoxyacetic acid (0.105 mL, 1.37 mmole) was added. EDC·HCl (0.245 g, 1.28 mmole) and N,N-diisopropylethylamine (0.370 mL, 2.12 mmole) were added and the mixture was stirred at RT for 18 hours. Water (25 mL) was added and the mixture was extracted with DCM (3×10 mL). The combined organics were dried over magnesium sulfate, filtered and concentrated. The residue was dissolved in DCM (2.1 mL) and imidazole (0.082 g, 1.20 mmole) and tert-butyldimethylchlorosilane (0.175 g, 1.16 mmole) were added. The mixture was stirred at RT for 18 hours and water (25 mL) was added. The mixture was extracted with DCM (3×10 mL) and the combined organics were dried over magnesium sulfate, filtered and concentrated. The crude residue was purified by silica gel chromatography to provide methyl O-(tert-butyldimethylsilyl)-N-(2-(4-(2-methoxyacetamido)piperidin-1-yl)thiazole-4-carbonyl)-L-serinate as a white solid (0.325 g, 60%).

¹H NMR (400 MHz, CDCl₃): δ 7.95 (d, J=8.8 Hz, 1H), 7.39 (s, 1H), 6.47 (d, J=8.2 Hz, 1H), 4.78 (ddd, J=8.9, 3.3, 2.6 Hz, 1H), 4.20-4.14 (m, 1H), 4.10-4.05 (m, 1H), 4.05-3.92 (m, 2H), 3.91-3.86 (m, 1H), 3.89 (s, 2H), 3.76 (s, 3H), 3.43 (s, 3H), 3.17 (dddd, J=13.2, 11.3, 8.1, 2.9 Hz, 2H), 2.07-1.99 (m, 2H), 1.57 (tddd, J=11.5, 10.2, 5.8, 4.4 Hz, 2H), 0.88 (s, 9H), 0.05 (s, 3H), 0.03 (s, 3H).

### Methyl O-acetyl-N—(O-(tert-butyldimethylsilyl)-N-(2-(4-(2-methoxyacetamido)piperidin-1-yl)thiazole-4-carbonyl)-L-seryl)-L-serinate (55.2)

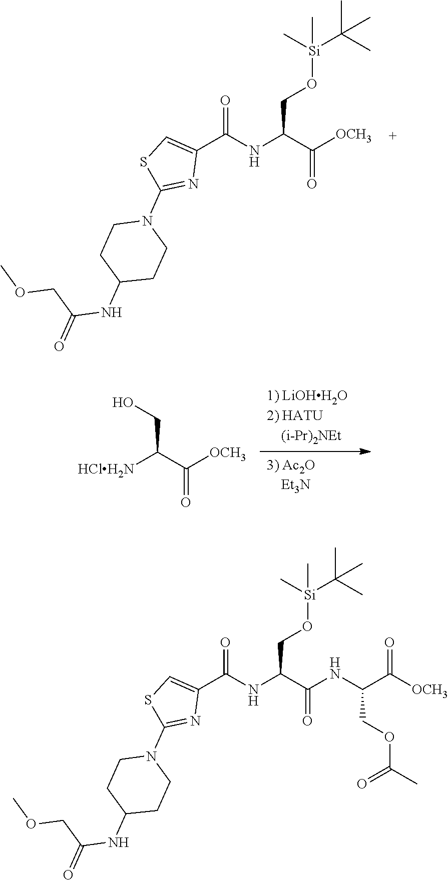

Methyl O-(tert-butyldimethylsilyl)-N-(2-(4-(2-methoxyacetamido)piperidin-1-yl)thiazole-4-carbonyl)-L-serinate (0.325 g, 0.631 mmole) was dissolved in 4/1/1 THF/methanol/water (2 mL). Lithium hydroxide monohydrate (0.057 g, 1.36 mmole) was added and the mixture was stirred at RT for 3 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was dissolved in DCM (2 mL). L-Serine methyl ester hydrochloride (0.133 g, 0.855 mmole) was added followed by HATU (0.291 g, 0.765 mmole) and N,N-diisopropylethylamine (0.220 mL, 1.26 mmole). The mixture was stirred at RT for 18 hours and water (25 mL) was added. The mixture was extracted with DCM (3×10 mL) and the combined organics were dried over magnesium sulfate, filtered and concentrated. The residue was dissolved in DCM (2 mL). Triethylamine (0.098 mL, 0.699 mmole) and acetic anhydride (0.066 mL, 0.698 mmole) were added and the solution was stirred at RT for 18 hours. Water (25 mL) was added and the mixture was extracted with DCM (3×10 mL). The combined organics were dried over magnesium sulfate, filtered and concentrated. The crude residue was purified by silica gel chromatography to provide methyl O-acetyl-N—(O-(tert-butyldimethylsilyl)-N-(2-(4-(2-methoxyacetamido)piperidin-1-yl)thiazole-4-carbonyl)-L-seryl)-L-serinate as a thick, pale yellow gel (0.254 g, 63%).

¹H NMR (400 MHz, CDCl₃): δ 7.96 (d, J=7.1 Hz, 1H), 7.41 (d, J=7.7 Hz, 1H), 7.38 (d, J=2.8 Hz, 1H), 6.44 (d, J=8.2 Hz, 1H), 4.85 (dt, J=7.7, 3.8 Hz, 1H), 4.56 (td, J=7.2, 3.6 Hz, 1H), 4.50-4.40 (m, 1H), 4.29 (dd, J=11.4, 3.7 Hz, 1H), 4.20-4.13 (m, 1H), 4.13-4.01 (m, 1H), 3.96 (t, J=14.4 Hz, 2H), 3.87 (s, 2H), 3.74 (d, J=2.1 Hz, 3H), 3.72-3.65 (m, 1H), 3.40 (s, 3H), 3.14 (tdd, J=13.0, 6.1, 3.0 Hz, 2H), 2.06-1.97 (m, 2H), 2.00 (s, 3H), 1.57-1.47 (m, 2H), 0.90 (s, 9H), 0.11 (s, 3H), 0.10 (s, 3H).

### Methyl 2-(2-(2-(4-(2-methoxyacetamido)piperidin-1-yl)thiazole-4-carboxamido)acrylamido)acrylate (55)

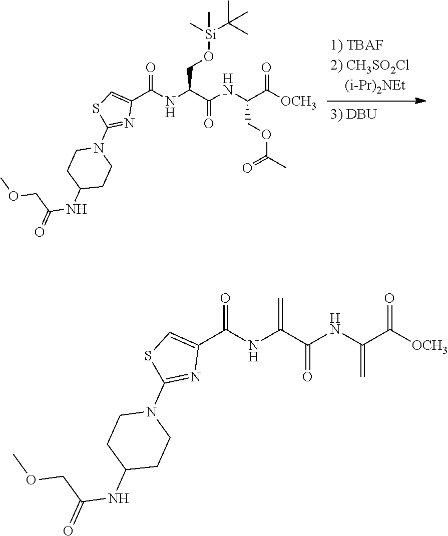 [SMILES: COCC(=O)NC1CCN(c2nc(C(=O)N[C@@H](CO[Si](C)(C)C(C)(C)C)C(=O)N[C@@H](COC(C)=O)C(=O)OC)cs2)CC1 ; C=C(NC(=O)c1csc(N2CCC(NC(=O)COC)CC2)n1)C(=O)NC(=C)C(=O)OC]

Methyl O-acetyl-N—(O-(tert-butyldimethylsilyl)-N-(2-(4-(2-methoxyacetamido)piperidin-1-yl)thiazole-4-carbonyl)-L-seryl)-L-serinate (0.254 g, 0.395 mmole) was dissolved in THF (2 mL). TBAF (1M solution in THF, 1.00 mL, 1.00 mmole) was added and the solution was stirred at RT for 18 hours. Water (25 mL) was added and the mixture was extracted with ethyl acetate (3×10 mL). The combined organics were dried over magnesium sulfate, filtered and concentrated. The residue was dissolved in DCM (2 mL) and cooled to 0° C. Triethylamine (0.083 mL, 0.592 mmole) and methanesulfonyl chloride (0.046 mL, 0.594 mmole) were added and the mixture was stirred at 0° for 90 minutes. Water (25 mL) was added and the mixture was extracted with DCM (3×10 mL). The combined organics were dried over magnesium sulfate, filtered and concentrated. The residue was dissolved in THF (2 mL) and cooled to 0° C. DBU (0.090 mL, 0.602 mmole) was added and the mixture was stirred at 0° for 90 minutes. Water (25 mL) was added and the mixture was extracted with ethyl acetate (3×10 mL). The combined organics were dried over magnesium sulfate, filtered and concentrated. The crude residue was purified by silica gel chromatography to provide methyl 2-(2-(2-(4-(2-methoxyacetamido)piperidin-1-yl)thiazole-4-carboxamido)acrylamido)acrylate as a white solid (0.093 g, 52%).

¹H NMR (400 MHz, CDCl₃): δ 9.72 (s, 1H), 8.51 (s, 1H), 7.43 (s, 1H), 6.72 (d, J=2.1 Hz, 1H), 6.66 (s, 1H), 6.47 (d, J=8.3 Hz, 1H), 6.01 (d, J=1.3 Hz, 1H), 5.44 (t, J=1.8 Hz, 1H), 4.10 (ddd, J=14.2, 7.3, 3.5 Hz, 1H), 4.01 (dt, J=13.4, 3.9 Hz, 2H), 3.90 (s, 2H), 3.89 (s, 3H), 3.42 (s, 3H), 3.20 (ddd, J=13.3, 11.7, 3.0 Hz, 2H), 2.12-2.00 (m, 2H), 1.66-1.52 (m, 2H).

### Compound 56: Tert-butyl (cis)-4-(4-((3-((3-methoxy-3-oxoprop-1-en-2-yl)amino)-3-oxoprop-1-en-2-yl)carbamoyl)thiazol-2-yl)-2,6-dimethylpiperazine-1-carboxylate

### Ethyl 2-((cis)-4-(tert-butoxycarbonyl)-3,5-dimethylpiperazin-1-yl)thiazole-4-carboxylate (56.1)

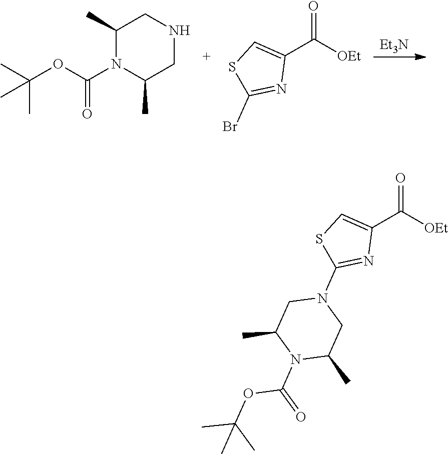 [SMILES: "Et3N" ; C[C@@H]1CNC[C@H](C)N1C(=O)OC(C)(C)C ; CCOC(=O)c1csc(Br)n1 ; CCOC(=O)c1csc(N2C[C@@H](C)N(C(=O)OC(C)(C)C)[C@@H](C)C2)n1]

Tert-butyl (cis)-2,6-dimethylpiperazine-1-carboxylate (1.033 g, 4.82 mmole) was dissolved in DMA (5 mL). Ethyl 2-bromothiazole-4-carboxylate (1.109 g, 4.70 mmole) and triethylamine (0.720 mL, 5.14 mmole) were added and the mixture was heated to 80° C. for 24 hours, then was cooled to RT and diluted with ethyl acetate. The organics were washed with water (3×15 mL) and dried over magnesium sulfate, then filtered and concentrated. The crude residue was purified by silica gel chromatography to provide ethyl 2-((cis)-4-(tert-butoxycarbonyl)-3,5-dimethylpiperazin-1-yl)thiazole-4-carboxylate as a pale yellow solid (0.699 g, 39%).

¹H NMR (400 MHz, CDCl₃): δ 7.45 (s, 1H), 4.35 (q, J=7.1 Hz, 2H), 4.29 (tdt, J=6.9, 4.7, 1.9 Hz, 2H), 3.86-3.76 (m, 2H), 3.26 (dd, J=12.7, 4.7 Hz, 2H), 1.49 (s, 9H), 1.37 (t, J=7.1 Hz, 3H), 1.31 (d, J=6.8 Hz, 6H).

### Tert-butyl (cis)-4-(4-(((S)-3-((tert-butyldimethylsilyl)oxy)-1-methoxy-1-oxopropan-2-yl)carbamoyl)thiazol-2-yl)-2,6-dimethylpiperazine-1-carboxylate (56.2)

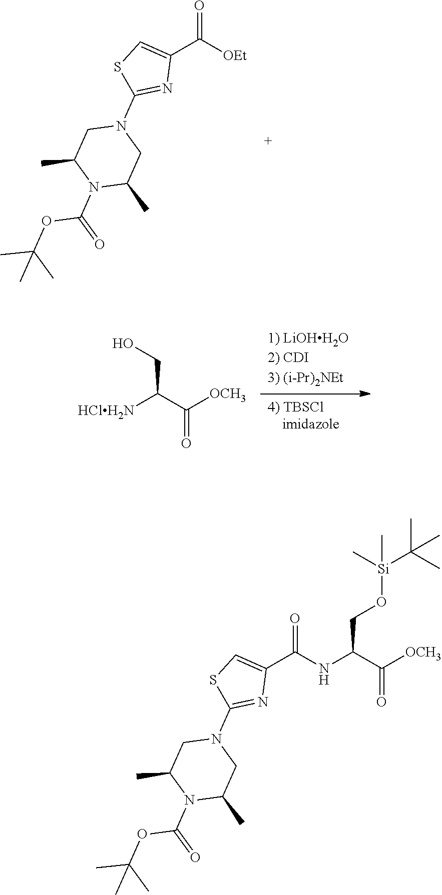

Ethyl 2-((cis)-4-(tert-butoxycarbonyl)-3,5-dimethylpiperazin-1-yl)thiazole-4-carboxylate (0.699 g, 1.89 mmole) was dissolved in 4/1/1 THF/methanol/water (3 mL). Lithium hydroxide monohydrate (0.167 g, 3.98 mmole) was added and the mixture was stirred at RT for 3 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was dissolved in THF (4 mL) and CDI (0.330 g, 2.04 mmole) was added portionwise (gas evolution was observed). The solution was stirred at RT for 60 minutes and L-serine methyl ester hydrochloride (0.382 g, 2.46 mmole) was added, followed by N,N-diisopropylethylamine (0.660 mL, 3.79 mmole). The mixture was stirred at RT for 3 days and water (25 mL) was added. The mixture was extracted with ethyl acetate (3×10 mL) and the combined organics were dried over magnesium sulfate, filtered and concentrated. The residue was dissolved in DCM (4 mL). Imidazole (0.150 g, 2.20 mmole) and tert-butyldimethylchlorosilane (0.336 g, 2.23 mmole) were added and the mixture was stirred at RT for 3 hours. Water (25 mL) was added and the mixture was extracted with DCM (3×10 mL). The combined organics were dried over magnesium sulfate, filtered and concentrated. The crude residue was purified by silica gel chromatography to provide tert-butyl (cis)-4-(4-(((S)-3-((tert-butyldimethylsilyl)oxy)-1-methoxy-1-oxopropan-2-yl)carbamoyl)thiazol-2-yl)-2,6-dimethylpiperazine-1-carboxylate as a colorless oil (0.849 g, 81%).

¹H NMR (400 MHz, CDCl₃): δ 7.93 (d, J=8.7 Hz, 1H), 7.39 (s, 1H), 4.79 (dt, J=8.8, 3.0 Hz, 1H), 4.29 (qdd, J=6.8, 4.8, 1.5 Hz, 2H), 4.20-4.14 (m, 1H), 3.89 (dd, J=10.1, 3.3 Hz, 1H), 3.85-3.71 (m, 2H), 3.77 (s, 3H), 3.24 (dd, J=12.7, 4.6 Hz, 2H), 1.50 (s, 9H), 1.35-1.27 (m, 6H), 0.88 (s, 9H), 0.06 (s, 3H), 0.03 (s, 3H).

### Tert-butyl 4-(4-(((cis)-9-(methoxycarbonyl)-2,2,3,3-tetramethyl-7,12-dioxo-4,11-dioxa-8-aza-3-silatridecan-6-yl)carbamoyl)thiazol-2-yl)-2,6-dimethylpiperazine-1-carboxylate (56.3)

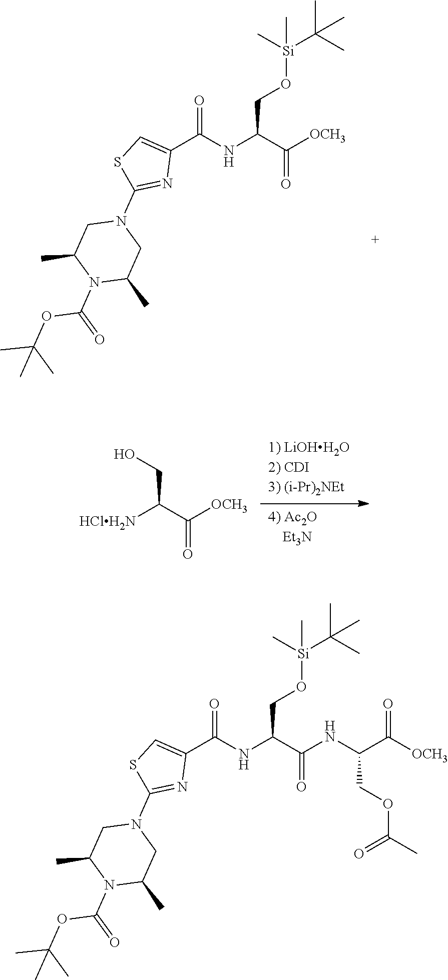

Tert-butyl (cis)-4-(4-(((S)-3-((tert-butyldimethylsilyl)oxy)-1-methoxy-1-oxopropan-2-yl)carbamoyl)thiazol-2-yl)-2,6-dimethylpiperazine-1-carboxylate (0.849 g, 1.52 mmole) was dissolved in 4/1/1 THF/methanol/water (3 mL). Lithium hydroxide monohydrate (0.126 g, 3.00 mmole) was added and the mixture was stirred at RT for 2 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was dissolved in THF (3 mL) and CDI (0.263 g, 1.62 mmole) was added portionwise (gas evolution was observed). The solution was stirred at RT for 60 minutes before L-serine methyl ester hydrochloride (0.306 g, 1.97 mmole) and N,N-diisopropylethylamine (0.530 mL, 3.04 mmole) were added. The mixture was stirred at RT for 18 hours and water (25 mL) was added. The mixture was extracted with DCM (3×10 mL). The combined organics were dried over magnesium sulfate, filtered and concentrated. The residue was dissolved in DCM (3 mL) and triethylamine (0.235 mL, 1.68 mmole) and acetic anhydride (0.160 mL, 1.69 mmole) were added. The mixture was stirred at RT for 18 hours and water (25 mL) was added. The mixture was extracted with DCM (3×10 mL) and the combined organics were dried over magnesium sulfate, filtered and concentrated. The crude residue was purified by silica gel chromatography to provide tert-butyl 4-(4-(((cis)-9-(methoxycarbonyl)-2,2,3,3-tetramethyl-7,12-dioxo-4,11-dioxa-8-aza-3-silatridecan-6-yl)carbamoyl)thiazol-2-yl)-2,6-dimethylpiperazine-1-carboxylate as a thick, colorless oil (0.593 g, 57%).

¹H NMR (400 MHz, CDCl₃): δ 7.96 (d, J=7.2 Hz, 1H), 7.43 (d, J=7.7 Hz, 1H), 7.41 (s, 1H), 4.87 (tt, J=6.6, 3.8 Hz, 1H), 4.63-4.53 (m, 1H), 4.52-4.45 (m, 1H), 4.30 (ddt, J=12.5, 7.1, 4.6 Hz, 3H), 4.19 (ddd, J=9.9, 3.7, 1.7 Hz, 1H), 3.80 (dd, J=4.7, 2.1 Hz, 2H), 3.76 (s, 3H), 3.75-3.69 (m, 1H), 3.24 (ddd, J=13.0, 4.7, 2.0 Hz, 2H), 2.02 (s, 3H), 1.49 (s, 9H), 1.31 (dt, J=6.9, 2.0 Hz, 6H), 0.93 (s, 9H), 0.14 (s, 3H), 0.12 (s, 3H).

### Tert-butyl (cis)-4-(4-((3-((3-methoxy-3-oxoprop-1-en-2-yl)amino)-3-oxoprop-1-en-2-yl)carbamoyl)thiazol-2-yl)-2,6-dimethylpiperazine-1-carboxylate (56)

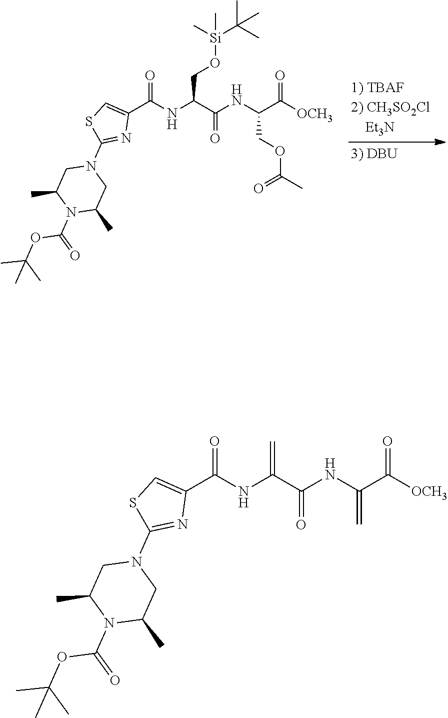 [SMILES: COC(=O)[C@H](COC(C)=O)NC(=O)[C@H](CO[Si](C)(C)C(C)(C)C)NC(=O)c1csc(N2C[C@@H](C)N(C(=O)OC(C)(C)C)[C@@H](C)C2)n1 ; C=C(NC(=O)c1csc(N2C[C@@H](C)N(C(=O)OC(C)(C)C)[C@@H](C)C2)n1)C(=O)NC(=C)C(=O)OC]

Tert-butyl 4-(4-(((cis)-9-(methoxycarbonyl)-2,2,3,3-tetramethyl-7,12-dioxo-4,11-dioxa-8-aza-3-silatridecan-6-yl)carbamoyl)thiazol-2-yl)-2,6-dimethylpiperazine-1-carboxylate (0.593 g, 0.865 mmole) was dissolved in THF (1.8 mL). TBAF (1M solution in THF, 2.20 mL, 2.20 mmole) was added and the solution was stirred at RT for 18 hours. Water (25 mL) was added and the mixture was extracted with ethyl acetate (3×10 mL). The combined organics were dried over magnesium sulfate, filtered and concentrated. The residue was dissolved in DCM (1.8 mL) and cooled to 0° C. Triethylamine (0.185 mL, 1.32 mmole) was added followed by methanesulfonyl chloride (0.100 mL, 1.29 mmole) and the mixture was stirred at 0° for 60 minutes. Water (25 mL) was added and the mixture was extracted with DCM (3×10 mL). The combined organics were dried over magnesium sulfate, filtered and concentrated. The residue was dissolved in THF (1.8 mL) and cooled to 0° C. DBU (0.200 mL, 1.34 mmole) was added and the mixture was stirred at 0° for 60 minutes. Water (25 mL) was added and the mixture was extracted with ethyl acetate (3×10 mL). The combined organics were dried over magnesium sulfate, filtered and concentrated. The crude residue was purified by silica gel chromatography to provide tert-butyl (cis)-4-(4-((3-((3-methoxy-3-oxoprop-1-en-2-yl)amino)-3-oxoprop-1-en-2-yl)carbamoyl)thiazol-2-yl)-2,6-dimethylpiperazine-1-carboxylate as a white solid (0.172 g, 40%).

¹H NMR (400 MHz, CDCl₃): δ 9.72 (s, 1H), 8.51 (s, 1H), 7.43 (s, 1H), 6.72 (d, J=2.2 Hz, 1H), 6.67 (s, 1H), 6.01 (d, J=1.3 Hz, 1H), 5.44 (t, J=1.9 Hz, 1H), 4.38-4.25 (m, 2H), 3.89 (s, 3H), 3.86-3.79 (m, 2H), 3.27 (dd, J=12.9, 4.6 Hz, 2H), 1.46 (s, 9H), 1.32 (d, J=6.9 Hz, 6H).

### Compound 57: Tert-butyl (S)-4-(4-((3-((3-methoxy-3-oxoprop-1-en-2-yl)amino)-3-oxoprop-1-en-2-yl)carbamoyl)thiazol-2-yl)-2-methylpiperazine-1-carboxylate

### Ethyl (S)-2-(4-(tert-butoxycarbonyl)-3-methylpiperazin-1-yl)thiazole-4-carboxylate (57.1)

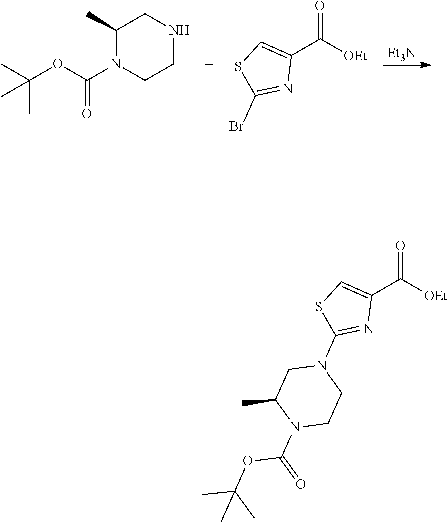 [SMILES: "Et3N" ; C[C@H]1CNCCN1C(=O)OC(C)(C)C ; CCOC(=O)c1csc(Br)n1 ; CCOC(=O)c1csc(N2CCN(C(=O)OC(C)(C)C)[C@@H](C)C2)n1]

Tert-butyl (S)-2-methylpiperazine-1-carboxylate (1.501 g, 7.49 mmole) was dissolved in DMA (7.5 mL). Ethyl 2-bromothiazole-4-carboxylate (1.864 g, 7.90 mmole) and triethylamine (1.30 mL, 9.28 mmole) were added and the mixture was stirred at 80° C. for 20 hours. The mixture was cooled to RT and diluted with ethyl acetate (25 mL). The mixture was washed with water (3×20 mL) and the organics were dried over magnesium sulfate, filtered and concentrated. The crude residue was purified by silica gel chromatography to provide ethyl (S)-2-(4-(tert-butoxycarbonyl)-3-methylpiperazin-1-yl)thiazole-4-carboxylate as an orange oil (1.842 g, 69%). ¹H NMR (400 MHz, CDCl₃): δ 7.45 (s, 1H), 4.42-4.34 (m, 1H), 4.35 (q, J=7.1 Hz, 2H), 4.04-3.91 (m, 2H), 3.68 (dt, J=12.8, 1.8 Hz, 1H), 3.38-3.30 (m, 1H), 3.24 (ddd, J=13.3, 11.9, 3.5 Hz, 1H), 3.09 (td, J=12.1, 3.7 Hz, 1H), 1.48 (s, 9H), 1.37 (t, J=7.1 Hz, 3H), 1.25-1.19 (m, 3H).

### Tert-butyl (S)-4-(4-(((S)-3-((tert-butyldimethylsilyl)oxy)-1-methoxy-1-oxopropan-2-yl)carbamoyl)thiazol-2-yl)-2-methylpiperazine-1-carboxylate (57.2)

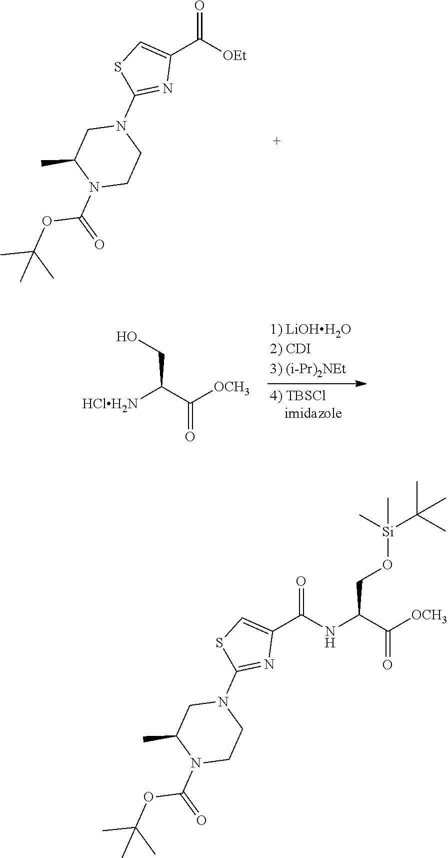

Ethyl (S)-2-(4-(tert-butoxycarbonyl)-3-methylpiperazin-1-yl)thiazole-4-carboxylate (0.605 g, 1.70 mmole) was dissolved in 4/1/1 THF/methanol/water (3.4 mL). Lithium hydroxide monohydrate (0.143 g, 3.41 mmole) was added and the mixture was stirred at RT for 2 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was dissolved in THF (3.4 mL) and CDI (0.291 g, 1.79 mmole) was added portionwise (gas evolution was observed). The solution was stirred at RT for 60 minutes before L-serine methyl ester hydrochloride (0.343 g, 2.20 mmole) and N,N-diisopropylethylamine (0.350 mL, 2.01 mmole) were added. The mixture was stirred at RT for 18 hours and water (25 mL) was added. The mixture was extracted with ethyl acetate (3×10 mL) and the combined organics were dried over magnesium sulfate, filtered and concentrated. The residue was dissolved in DCM (3.4 mL) and imidazole (0.126 g, 1.85 mmole) and tert-butyldimethylchlorosilane (0.285 g, 1.89 mmole) were added. The mixture was stirred at RT for 90 minutes and water (25 mL) was added. The mixture was extracted with DCM (3×10 mL) and the combined organics were dried over magnesium sulfate, filtered and concentrated. The crude residue was purified by silica gel chromatography to provide tert-butyl (S)-4-(4-(((S)-3-((tert-butyldimethylsilyl)oxy)-1-methoxy-1-oxopropan-2-yl)carbamoyl)thiazol-2-yl)-2-methylpiperazine-1-carboxylate as a thick, pale yellow gel (0.636 g, 69%).

¹H NMR (400 MHz, CDCl₃): δ 7.94 (d, J=8.7 Hz, 1H), 7.39 (s, 1H), 4.78 (ddd, J=8.8, 3.4, 2.6 Hz, 1H), 4.38 (s, 1H), 4.16 (dd, J=10.0, 2.6 Hz, 1H), 4.00-3.92 (m, 1H), 3.89 (dd, J=10.0, 3.4 Hz, 2H), 3.76 (s, 3H), 3.68 (dt, J=12.7, 1.9 Hz, 1H), 3.34-3.26 (m, 1H), 3.26-3.16 (m, 1H), 3.09 (td, J=12.1, 3.6 Hz, 1H), 1.49 (s, 9H), 1.25 (dd, J=7.0, 4.4 Hz, 3H), 0.89 (s, 9H), 0.05 (s, 3H), 0.03 (s, 3H).

### Tert-butyl (S)-4-(4-(((6S,9S)-9-(methoxycarbonyl)-2,2,3,3-tetramethyl-7,12-dioxo-4,11-dioxa-8-aza-3-silatridecan-6-yl)carbamoyl)thiazol-2-yl)-2-methylpiperazine-1-carboxylate (57.3)

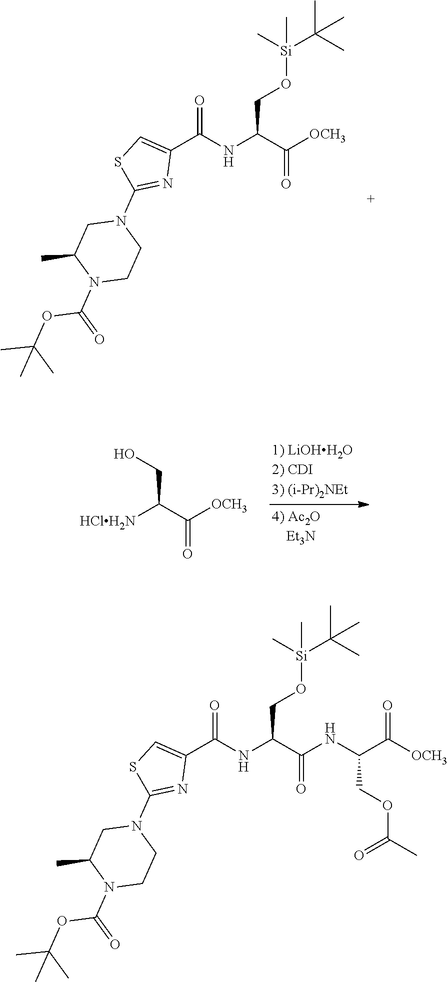

Tert-butyl (S)-4-(4-(((S)-3-((tert-butyldimethylsilyl)oxy)-1-methoxy-1-oxopropan-2-yl)carbamoyl)thiazol-2-yl)-2-methylpiperazine-1-carboxylate (0.636 g, 1.17 mmole) was dissolved in 4/1/1 THF/methanol/water (2.4 mL). Lithium hydroxide monohydrate (0.102 g, 2.43 mmole) was added and the mixture was stirred at RT for 3 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was suspended in THF (2.4 mL). CDI (0.202 g, 1.25 mmole) was added portionwise (gas evolution was observed) and the solution was stirred at RT for 45 minutes. L-Serine methyl ester hydrochloride (0.239 g, 1.54 mmole) was added followed by N,N-diisopropylethylamine (0.270 mL, 1.55 mmole) and the mixture was stirred at RT for 18 hours. Water (25 mL) was added and the mixture was extracted with ethyl acetate (3×10 mL). The combined organics were dried over magnesium sulfate, filtered and concentrated. The residue was dissolved in DCM (2.4 mL), then triethylamine (0.180 mL, 1.28 mmole) and acetic anhydride (0.120 mL, 1.27 mmole) were added. The mixture was stirred at RT for 3 hours and water (25 mL) was added. The mixture was extracted with DCM (3×10 mL) and the combined organics were dried over magnesium sulfate, filtered and concentrated. The crude residue was purified by silica gel chromatography to provide tert-butyl (S)-4-(4-(((6S,9S)-9-(methoxycarbonyl)-2,2,3,3-tetramethyl-7,12-dioxo-4,11-dioxa-8-aza-3-silatridecan-6-yl)carbamoyl)thiazol-2-yl)-2-methylpiperazine-1-carboxylate as a pale yellow oil (0.471 g, 60%).

¹H NMR (400 MHz, CDCl₃): δ 7.98 (d, J=7.0 Hz, 1H), 7.44 (d, J=7.8 Hz, 1H), 7.41 (d, J=2.8 Hz, 1H), 4.88 (dt, J=7.6, 3.7 Hz, 1H), 4.58 (td, J=7.2, 3.6 Hz, 1H), 4.47 (ddd, J=11.4, 10.1, 4.0 Hz, 1H), 4.42-4.35 (m, 1H), 4.31 (dd, J=11.4, 3.6 Hz, 1H), 4.19 (ddd, J=9.8, 3.6, 2.1 Hz, 1H), 3.99-3.87 (m, 2H), 3.83-3.70 (m, 1H), 3.77 (s, 3H), 3.68 (dd, J=12.8, 2.0 Hz, 1H), 3.30 (dd, J=12.9, 4.1 Hz, 1H), 3.28-3.17 (m, 1H), 3.13-3.04 (m, 1H), 2.02 (s, 3H), 1.49 (s, 9H), 1.25-1.21 (m, 3H), 0.93 (s, 9H), 0.14 (s, 3H), 0.12 (s, 3H).

### Tert-butyl (S)-4-(4-((3-((3-methoxy-3-oxoprop-1-en-2-yl)amino)-3-oxoprop-1-en-2-yl)carbamoyl)thiazol-2-yl)-2-methylpiperazine-1-carboxylate (57)

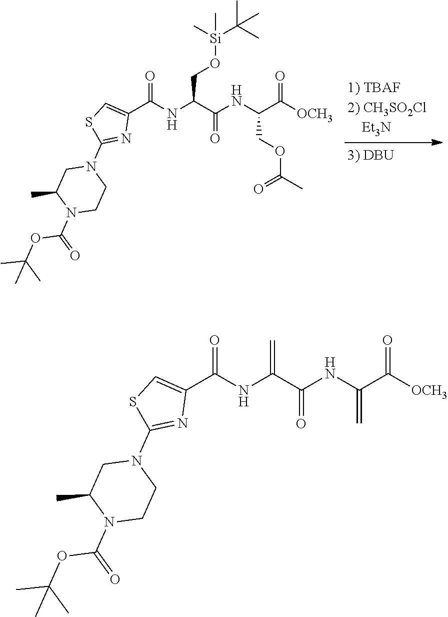 [SMILES: COC(=O)[C@H](COC(C)=O)NC(=O)[C@H](CO[Si](C)(C)C(C)(C)C)NC(=O)c1csc(N2CCN(C(=O)OC(C)(C)C)[C@@H](C)C2)n1 ; C=C(NC(=O)c1csc(N2CCN(C(=O)OC(C)(C)C)[C@@H](C)C2)n1)C(=O)NC(=C)C(=O)OC]

Tert-butyl (S)-4-(4-(((6S,9S)-9-(methoxycarbonyl)-2,2,3,3-tetramethyl-7,12-dioxo-4,11-dioxa-8-aza-3-silatridecan-6-yl)carbamoyl)thiazol-2-yl)-2-methylpiperazine-1-carboxylate (0.471 g, 0.701 mmole) was dissolved in THF (2.8 mL). TBAF (1M solution in THF, 1.80 mL, 1.80 mmole) was added and the solution was stirred at RT for 18 hours. Water (25 mL) was added and the mixture was extracted with ethyl acetate (3×10 mL). The combined organics were dried over magnesium sulfate, filtered and concentrated. The residue was dissolved in DCM (2.8 mL) and cooled to 0° C. Triethylamine (0.150 mL, 1.07 mmole) was added followed by methanesulfonyl chloride (0.082 mL, 1.06 mmole) and the mixture was stirred at 0° for 60 minutes. Water (25 mL) was added and the mixture was extracted with DCM (3×10 mL). The combined organics were dried over magnesium sulfate, filtered and concentrated. The residue was dissolved in THF (2.8 mL) and cooled to 0° C. DBU (0.160 mL, 1.07 mmole) was added and the mixture was stirred at 0° for 60 minutes. Water (25 mL) was added and the mixture was extracted with ethyl acetate (3×10 mL). The combined organics were dried over magnesium sulfate, filtered and concentrated. The crude residue was purified by silica gel chromatography to provide tert-butyl (S)-4-(4-((3-((3-methoxy-3-oxoprop-1-en-2-yl)amino)-3-oxoprop-1-en-2-yl)carbamoyl)thiazol-2-yl)-2-methylpiperazine-1-carboxylate as a white solid (0.115 g, 34%).

¹H NMR (400 MHz, CDCl₃): δ 9.72 (s, 1H), 8.51 (s, 1H), 7.44 (s, 1H), 6.72 (d, J=2.1 Hz, 1H), 6.67 (s, 1H), 6.01 (d, J=1.3 Hz, 1H), 5.44 (t, J=1.9 Hz, 1H), 4.41 (s, 1H), 4.04-3.93 (m, 2H), 3.89 (s, 3H), 3.70 (dt, J=12.8, 1.8 Hz, 1H), 3.32 (dd, J=12.7, 4.1 Hz, 1H), 3.24 (ddd, J=13.5, 11.9, 3.6 Hz, 1H), 3.11 (td, J=12.2, 3.8 Hz, 1H), 1.49 (s, 9H), 1.25 (d, J=7.0, 3H).

### Compound 58: Methyl 2-(2-(2-(4-(1,4-dioxane-2-carboxamido)piperidin-1-yl)thiazole-4-carboxamido)acrylamido)acrylate

### Methyl N-(2-(4-(1,4-dioxane-2-carboxamido)piperidin-1-yl)thiazole-4-carbonyl)-O-(tert-butyldimethylsilyl)-L-serinate (58.1)

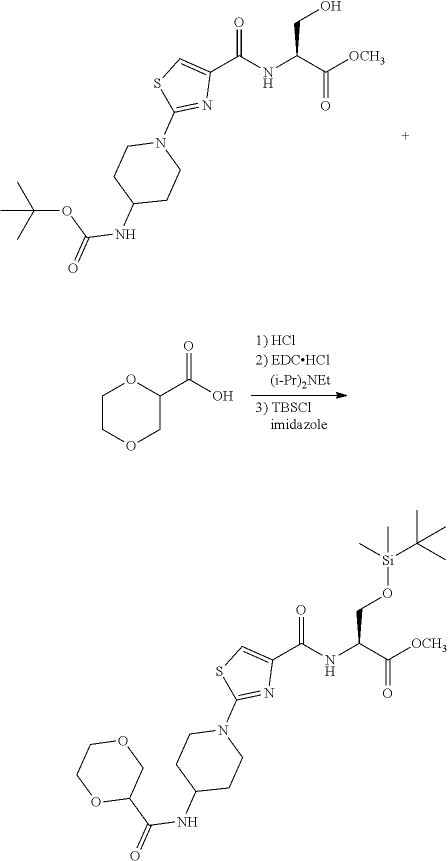 [SMILES: COC(=O)[C@H](CO)NC(=O)c1csc(N2CCC(NC(=O)OC(C)(C)C)CC2)n1 ; O=C(O)C1COCCO1 ; COC(=O)[C@H](CO[Si](C)(C)C(C)(C)C)NC(=O)c1csc(N2CCC(NC(=O)C3COCCO3)CC2)n1]

Methyl (2-(4-((tert-butoxycarbonyl)amino)piperidin-1-yl)thiazole-4-carbonyl)-L-serinate (Example 46.2, 0.507 g, 1.18 mmole) was dissolved in DCM (5 ML). HCl (4M solution in 1,4-dioxane, 1.20 mL, 4.80 mmole) was added and the mixture was stirred at RT for 2 hours, then was concentrated. The residue was suspended in DCM (2.4 mL) and 1,4-dioxane-2-carboxylic acid (0.199 g, 1.51 mmole) was added. EDC·HCl (0.289 g, 1.51 mmole) and N,N-diisopropylethylamine (0.270 mL, 1.55 mmole) were added and the mixture was stirred at RT for 3 days. Water (25 mL) was added and the mixture was extracted with DCM (3×10 mL). The combined organics were dried over magnesium sulfate, filtered and concentrated. The residue was dissolved in DCM (2.4 mL). Imidazole (0.090 g, 1.32 mmole) and tert-butyldimethylchlorosilane (0.196 g, 1.30 mmole) were added and the mixture was stirred at RT for 3 hours. Water (25 mL) was added and the mixture was extracted with DCM (3×10 mL). The combined organics were dried over magnesium sulfate, filtered and concentrated. The crude residue was purified by silica gel chromatography to provide methyl N-(2-(4-(1,4-dioxane-2-carboxamido)piperidin-1-yl)thiazole-4-carbonyl)-O-(tert-butyldimethylsilyl)-L-serinate as a thick, pale yellow oil (0.321 g, 49%).

¹H NMR (400 MHz, CDCl₃): δ 7.95 (d, J=8.8 Hz, 1H), 7.39 (s, 1H), 6.48 (d, J=8.2 Hz, 1H), 4.79 (dt, J=8.9, 3.0 Hz, 1H), 4.18 (dd, J=4.9, 2.9 Hz, 2H), 4.12-4.07 (m, 1H), 4.01 (ddt, J=12.4, 8.6, 3.8 Hz, 2H), 3.97-3.90 (m, 1H), 3.90-3.83 (m, 2H), 3.83-3.71 (m, 2H), 3.76 (s, 3H), 3.66-3.55 (m, 1H), 3.42 (dd, J=11.5, 10.1 Hz, 1H), 3.23-3.10 (m, 2H), 2.03-1.96 (m, 2H), 1.63-1.49 (m, 2H), 0.88 (s, 9H), 0.05 (s, 3H), 0.03 (s, 3H).

### Methyl N—(N-(2-(4-(1,4-dioxane-2-carboxamido)piperidin-1-yl)thiazole-4-carbonyl)-O-(tert-butyldimethylsilyl)-L-seryl)-O-acetyl-L-serinate (58.2)

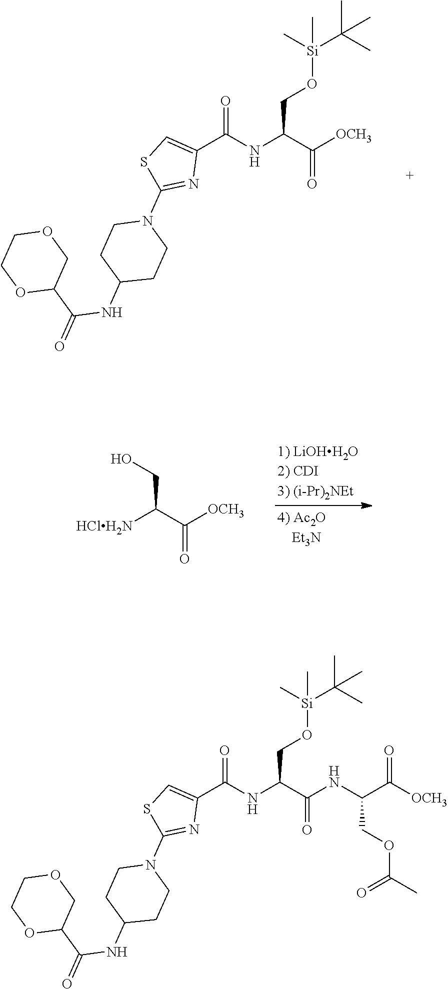

Methyl N-(2-(4-(1,4-dioxane-2-carboxamido)piperidin-1-yl)thiazole-4-carbonyl)-O-(tert-butyldimethylsilyl)-L-serinate (0.321 g, 0.577 mmole) was dissolved in 4/1/1 THF/methanol/water (2.4 mL). Lithium hydroxide monohydrate (0.049 g, 1.17 mmole) was added and the mixture was stirred at RT for 2 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was dissolved in THF (2.4 mL). CDI (0.103 g, 0.635 mmole) was added portionwise and the mixture was stirred at RT for 60 minutes. L-Serine methyl ester hydrochloride (0.124 g, 0.797 mmole) was added followed by N,N-diisopropylethylamine (0.130 mL, 0.746 mmole). The mixture was stirred at RT for 18 hours and water (25 mL) was added. The mixture was extracted with ethyl acetate (3×10 mL) and the combined organics were dried over magnesium sulfate, filtered and concentrated. The residue was dissolved in DCM (2.4 mL). Triethylamine (0.090 mL, 0.642 mmole) and acetic anhydride (0.060 mL, 0.635 mmole) were added and the solution was stirred at RT for 18 hours. Water (25 mL) was added and the mixture was extracted with DCM (3×10 mL). The combined organics were dried over magnesium sulfate, filtered and concentrated. The crude residue was purified by silica gel chromatography to provide methyl N—(N-(2-(4-(1,4-dioxane-2-carboxamido)piperidin-1-yl)thiazole-4-carbonyl)-O-(tert-butyldimethylsilyl)-L-seryl)-O-acetyl-L-serinate as a thick, pale yellow oil (0.197 g, 50%).

¹H NMR (400 MHz, CDCl₃): δ 7.98 (d, J=7.1 Hz, 1H), 7.44 (d, J=7.8 Hz, 1H), 7.40 (d, J=2.5 Hz, 1H), 6.48 (d, J=8.2 Hz, 1H), 4.87 (dq, J=8.4, 4.6, 4.1 Hz, 1H), 4.58 (td, J=7.3, 3.6 Hz, 1H), 4.47 (ddd, J=11.1, 9.0, 4.0 Hz, 1H), 4.32 (dd, J=11.4, 3.7 Hz, 1H), 4.22-4.14 (m, 2H), 4.11 (dd, J=7.1, 2.9 Hz, 1H), 4.07-3.90 (m, 3H), 3.90-3.78 (m, 2H), 3.76 (s, 3H), 3.74-3.69 (m, 2H), 3.60 (td, J=11.5, 3.1 Hz, 1H), 3.42 (dd, J=11.5, 10.1 Hz, 1H), 3.24-3.09 (m, 2H), 2.06-1.97 (m, 2H), 2.02 (s, 3H), 1.65-1.48 (m, 2H), 0.92 (s, 9H), 0.14 (s, 3H), 0.12 (s, 3H).

### Methyl 2-(2-(2-(4-(1,4-dioxane-2-carboxamido)piperidin-1-yl)thiazole-4-carboxamido)acrylamido)acrylate (58)

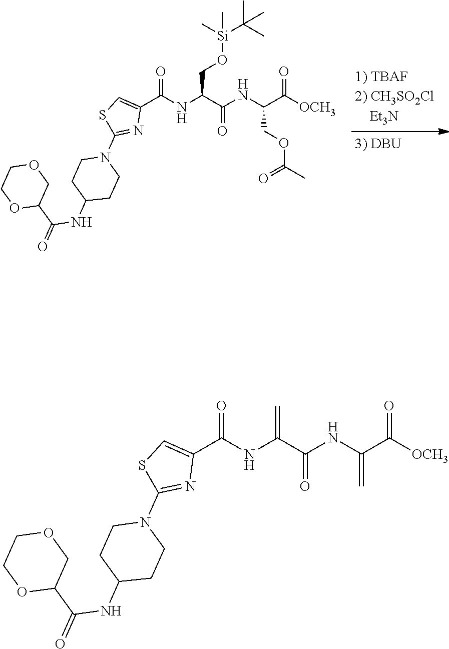 [SMILES: COC(=O)[C@H](COC(C)=O)NC(=O)[C@H](CO[Si](C)(C)C(C)(C)C)NC(=O)c1csc(N2CCC(NC(=O)C3COCCO3)CC2)n1 ; C=C(NC(=O)c1csc(N2CCC(NC(=O)C3COCCO3)CC2)n1)C(=O)NC(=C)C(=O)OC]

Methyl N—(N-(2-(4-(1,4-dioxane-2-carboxamido) piperidin-1-yl) thiazole-4-carbonyl)-O-(tert-butyldimethylsilyl)-L-seryl)-O-acetyl-L-serinate (0.197 g, 0.287 mmole) was dissolved in THF (2 mL). TBAF (1M solution in THF, 0.720 mL, 0.720 mmole) was added and the solution was stirred at RT for 18 hours. Water (25 mL) and sat. aq. sodium chloride (15 mL) were added and the mixture was extracted with ethyl acetate (3×10 mL). The combined organics were dried over magnesium sulfate, filtered and concentrated. The residue was dissolved in DCM (2 mL) and cooled to 0° C. Triethylamine (0.060 mL, 0.428 mmole) and methanesulfonyl chloride (0.034 mL, 0.439 mmole) were added and the mixture was stirred at 0° for 60 minutes. Water (25 mL) was added and the mixture was extracted with DCM (3×10 mL). The combined organics were dried over magnesium sulfate, filtered and concentrated. The residue was dissolved in THF (2 mL) and cooled to 0° C. DBU was added and the mixture was stirred at 0° for 60 minutes. Water (25 mL) and sat. aq. sodium chloride (15 mL) were added and the mixture was extracted with ethyl acetate (3×10 mL). The combined organics were dried over magnesium sulfate, filtered and concentrated. The crude residue was purified by silica gel chromatography to provide methyl 2-(2-(2-(4-(1,4-dioxane-2-carboxamido)piperidin-1-yl)thiazole-4-carboxamido)acrylamido)acrylate as a white solid (0.047 g, 33%).

¹H NMR (400 MHz, CDCl₃): δ 9.71 (s, 1H), 8.51 (s, 1H), 7.44 (s, 1H), 6.72 (d, J=2.1 Hz, 1H), 6.66 (s, 1H), 6.48 (d, J=8.2 Hz, 1H), 6.01 (d, J=1.3 Hz, 1H), 5.44 (t, J=1.9 Hz, 1H), 4.14 (ddd, J=22.7, 10.8, 3.1 Hz, 2H), 4.02 (dddd, J=22.7, 11.8, 5.6, 2.6 Hz, 3H), 3.89 (s, 3H), 3.86-3.71 (m, 3H), 3.66-3.53 (m, 1H), 3.42 (dd, J=11.5, 10.1 Hz, 1H), 3.20 (ddd, J=13.4, 11.6, 3.0 Hz, 2H), 2.10-1.98 (m, 2H), 1.65-1.51 (m, 2H).

### Compound 59: Methyl 2-(2-(2-(4-(tetrahydro-2H-pyran-4-carbonyl)piperazin-1-yl)thiazole-4-carboxamido)acrylamido)acrylate

### Methyl O-(tert-butyldimethylsilyl)-N-(2-(4-(tetrahydro-2H-pyran-4-carbonyl)piperazin-1-yl)thiazole-4-carbonyl)-L-serinate (59.1)

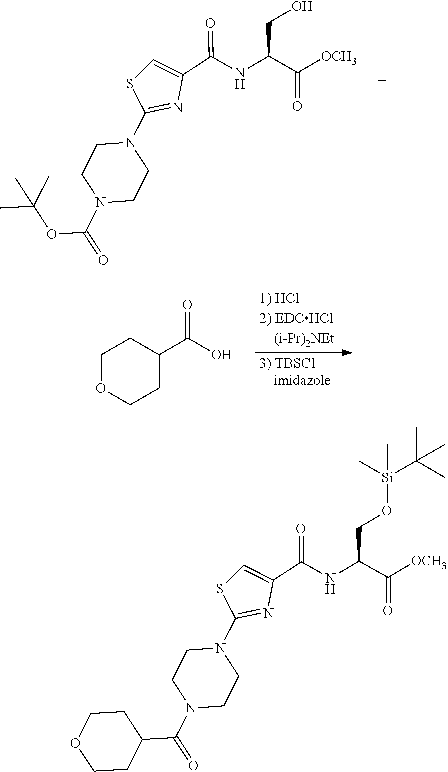 [SMILES: COC(=O)[C@H](CO)NC(=O)c1csc(N2CCN(C(=O)OC(C)(C)C)CC2)n1 ; O=C(O)C1CCOCC1 ; COC(=O)[C@H](CO[Si](C)(C)C(C)(C)C)NC(=O)c1csc(N2CCN(C(=O)C3CCOCC3)CC2)n1]

Tert-butyl (S)-4-(4-((3-hydroxy-1-methoxy-1-oxopropan-2-yl)carbamoyl)thiazol-2-yl)piperazine-1-carboxylate (Example 47.2, 0.749 g, 1.81 mmole) was dissolved in DCM (3.6 mL). HCl (4M solution in 1,4-dioxane, 1.80 mL, 7.20 mmole) was added and the mixture was stirred at RT for 2 hours, then was concentrated. The residue was suspended in DCM (3.6 mL) and tetrahydro-2H-pyran-4-carboxylic acid (0.315 g, 2.42 mmole) was added. EDC·HCl (0.455 g, 2.37 mmole) was added, followed by N,N-diisopropylethylamine (0.630 mL, 3.62 mmole) and the mixture was stirred at RT for 2 days. Water (25 mL) was added and the mixture was extracted with DCM (3×10 mL). The combined organics were dried over magnesium sulfate, filtered and concentrated. The residue was dissolved in DCM (3.6 mL) and imidazole (0.135 g, 1.98 mmole) was added, followed by tert-butyldimethylchlorosilane (0.300 g, 1.99 mmole). The mixture was stirred at RT for 18 hours and water (25 mL) was added. The mixture was extracted with DCM (3×10 mL) and the combined organics were dried over magnesium sulfate, filtered and concentrated. The crude residue was purified by silica gel chromatography to provide methyl O-(tert-butyldimethylsilyl)-N-(2-(4-(tetrahydro-2H-pyran-4-carbonyl)piperazin-1-yl)thiazole-4-carbonyl)-L-serinate as a thick, pale yellow oil (0.543 g, 55%).

¹H NMR (400 MHz, CDCl₃): δ 7.93 (d, J=8.7 Hz, 1H), 7.45 (s, 1H), 4.79 (dt, J=8.7, 3.0 Hz, 1H), 4.16 (dd, J=8.3, 1.8 Hz, 1H), 4.05 (ddd, J=11.8, 4.3, 2.3 Hz, 2H), 3.90 (dd, J=10.0, 3.3 Hz, 1H), 3.82-3.71 (m, 2H), 3.77 (s, 3H), 3.66 (s, 2H), 3.59-3.42 (m, 6H), 2.76 (tt, J=11.3, 3.8 Hz, 1H), 2.02-1.88 (m, 2H), 1.67-1.59 (m, 2H), 0.88 (s, 9H), 0.05 (s, 3H), 0.03 (s, 3H).

### Methyl O-acetyl-N—(O-(tert-butyldimethylsilyl)-N-(2-(4-(tetrahydro-2H-pyran-4-carbonyl)piperazin-1-yl)thiazole-4-carbonyl)-L-seryl)-L-serinate (59.2)

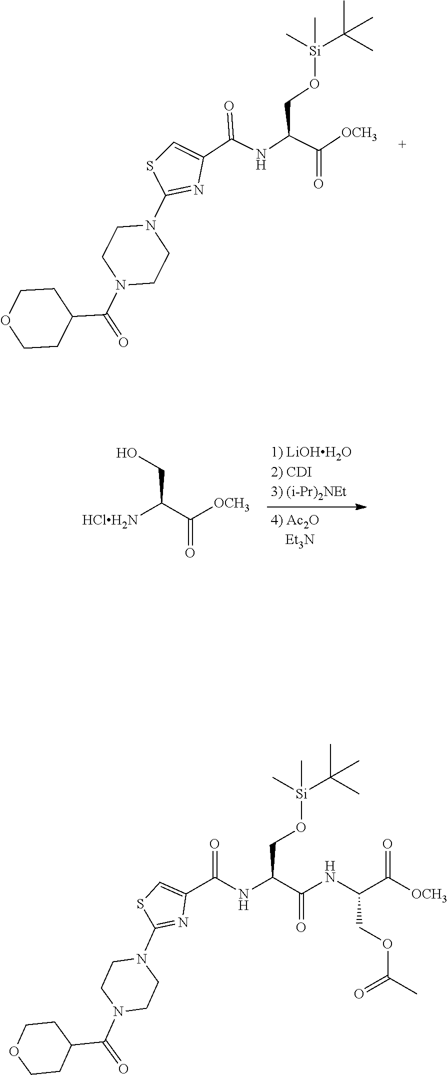

Methyl O-(tert-butyldimethylsilyl)-N-(2-(4-(tetrahydro-2H-pyran-4-carbonyl)piperazin-1-yl)thiazole-4-carbonyl)-L-serinate (0.543 g, 1.00 mmole) was dissolved in 4/1/1 THF/methanol/water (2 mL). Lithium hydroxide monohydrate (0.069 g, 1.54 mmole) was added and the mixture was stirred at RT for 2 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was dissolved in THF (2 mL) and CDI (0.172 g, 1.06 mmole) was added portionwise (gas evolution was observed). The solution was stirred at RT for 60 minutes and L-serine methyl ester hydrochloride (0.205 g, 1.32 mmole) was added, followed by N,N-diisopropylethylamine (0.230 mL, 1.32 mmole). The solution was stirred at RT for 18 hours and water (25 mL) was added. The mixture was extracted with ethyl acetate (3×10 mL) and the combined organics were dried over magnesium sulfate, filtered and concentrated. The residue was dissolved in DCM (2 mL) and triethylamine (0.155 mL, 1.11 mmole) and acetic anhydride (0.105 mL, 1.11 mmole) were added. The solution was stirred at RT for 18 hours and water (25 mL) was added. The mixture was extracted with DCM (3×10 mL) and the combined organics were dried over magnesium sulfate, filtered and concentrated. The crude residue was purified by silica gel chromatography (Isco CombiPrep, 12 g RediSep column, 70-100% ethyl acetate/hexane gradient)

### Methyl 2-(2-(2-(4-(tetrahydro-2H-pyran-4-carbonyl)piperazin-1-yl)thiazole-4-carboxamido)acrylamido)acrylate (59)

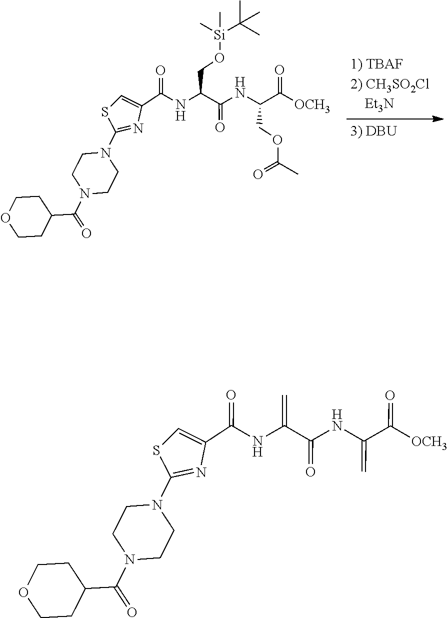 [SMILES: COC(=O)[C@H](COC(C)=O)NC(=O)[C@H](CO[Si](C)(C)C(C)(C)C)NC(=O)c1csc(N2CCN(C(=O)C3CCOCC3)CC2)n1 ; C=C(NC(=O)c1csc(N2CCN(C(=O)C3CCOCC3)CC2)n1)C(=O)NC(=C)C(=O)OC]

Methyl O-acetyl-N—(O-(tert-butyldimethylsilyl)-N-(2-(4-(tetrahydro-2H-pyran-4-carbonyl)piperazin-1-yl)thiazole-4-carbonyl)-L-seryl)-L-serinate (0.353 g, 0.527 mmole) was dissolved in THF. TBAF (1M solution in THF, 1.30 mL, 1.30 mmole) was added and the solution was stirred at RT for 18 hours. Water (25 mL) and sat. aq. sodium chloride (15 mL) were added and the mixture was extracted with ethyl acetate (3×10 mL). The combined organics were dried over magnesium sulfate, filtered and concentrated. The residue was dissolved in DCM (2 mL) and cooled to 0° C. Triethylamine 0.110 mL, 0.785 mmole) and methanesulfonyl chloride (0.061 mL, 0.788 mmole) were added and the mixture was stirred at 0° for 60 minutes. Water (25 mL) was added and the mixture was extracted with DCM (3×10 mL). The combined organics were dried over magnesium sulfate, filtered and concentrated. The residue was dissolved in THF (2 mL) and cooled to 0° C. DBU (0.120 mL, 0.802 mmole) was added and the mixture was stirred at 0° for 60 minutes. Water (25 mL) and sat. aq. sodium chloride (15 mL) were added and the mixture was extracted with ethyl acetate (3×10 mL). The combined organics were dried over magnesium sulfate, filtered and concentrated. The crude residue was purified by silica gel chromatography to provide methyl 2-(2-(2-(4-(tetrahydro-2H-pyran-4-carbonyl)piperazin-1-yl)thiazole-4-carboxamido)acrylamido)acrylate as a white solid (0.097 g, 39%).

¹H NMR (400 MHz, CDCl₃): δ 9.79-9.69 (m, 1H), 8.53 (s, 1H), 7.49 (s, 1H), 6.73 (d, J=2.2 Hz, 1H), 6.65 (s, 1H), 6.01 (d, J=1.2 Hz, 1H), 5.45 (t, J=1.9 Hz, 1H), 4.05 (ddd, J=11.7, 4.3, 2.2 Hz, 2H), 3.90 (s, 3H), 3.79 (s, 2H), 3.73-3.58 (m, 4H), 3.47 (td, J=11.8, 2.1 Hz, 4H), 2.76 (tt, J=11.3, 3.8 Hz, 1H), 1.95 (dtd, J=13.7, 11.7, 4.4 Hz, 2H), 1.67-1.61 (m, 2H).

### Compound 60: Tert-butyl (R)-4-(4-((3-((3-methoxy-3-oxoprop-1-en-2-yl)amino)-3-oxoprop-1-en-2-yl)carbamoyl)thiazol-2-yl)-2-methylpiperazine-1-carboxylate

### Ethyl (R)-2-(4-(tert-butoxycarbonyl)-3-methylpiperazin-1-yl)thiazole-4-carboxylate (60.1)

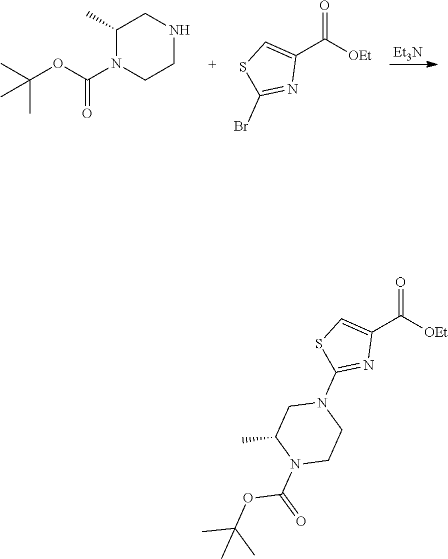 [SMILES: "Et3N" ; C[C@@H]1CNCCN1C(=O)OC(C)(C)C ; CCOC(=O)c1csc(Br)n1 ; CCOC(=O)c1csc(N2CCN(C(=O)OC(C)(C)C)[C@H](C)C2)n1]

Tert-butyl (R)-2-methylpiperazine-1-carboxylate (1.499 g, 7.48 mmole) was dissolved in DMA (7.5 mL). Ethyl 2-bromothiazole (1.780 g, 7.54 mmole) was added followed by triethylamine (1.30 mL, 9.28 mmole). The mixture was heated to 80° C. for 28 hours, then was cooled to RT and diluted with ethyl acetate (25 mL). The mixture was washed with water (3×25 mL) and the organics were dried over magnesium sulfate, filtered and concentrated. The crude residue was purified by silica gel chromatography to provide ethyl (R)-2-(4-(tert-butoxycarbonyl)-3-methylpiperazin-1-yl)thiazole-4-carboxylate as a thick, orange gel (1.515 g, 57%).

¹H NMR (400 MHz, CDCl₃): δ 7.45 (s, 1H), 4.42-4.35 (m, 1H), 4.35 (q, J=7.1 Hz, 2H), 4.03-3.90 (m, 2H), 3.68 (dt, J=12.8, 1.9 Hz, 1H), 3.34 (dd, J=12.7, 4.1 Hz, 1H), 3.24 (ddd, J=13.3, 11.9, 3.5 Hz, 1H), 3.09 (td, J=12.1, 3.6 Hz, 1H), 1.48 (s, 9H), 1.37 (t, J=7.1 Hz, 3H), 1.23 (d, J=6.8 Hz, 3H).

### Tert-butyl (R)-4-(4-(((S)-3-((tert-butyldimethylsilyl)oxy)-1-methoxy-1-oxopropan-2-yl)carbamoyl)thiazol-2-yl)-2-methylpiperazine-1-carboxylate (60.2)

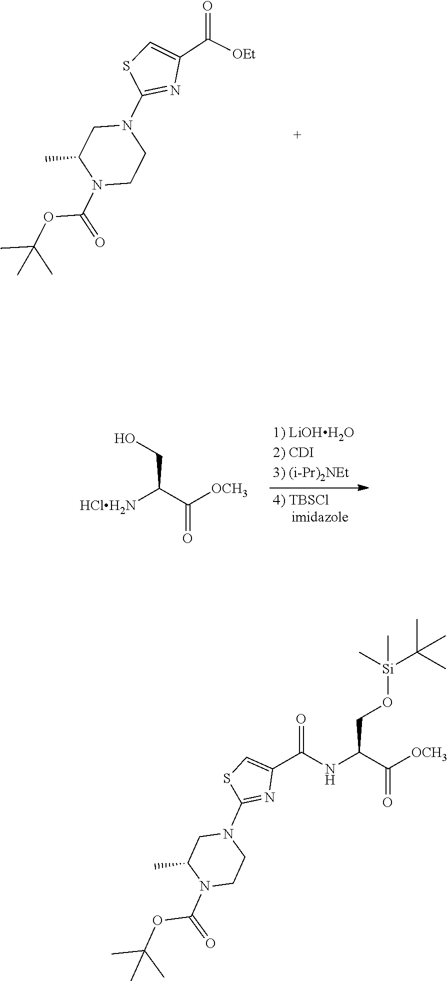

Ethyl (R)-2-(4-(tert-butoxycarbonyl)-3-methylpiperazin-1-yl)thiazole-4-carboxylate (0.605 g, 1.70 mmole) was dissolved in 4/1/1 THF/methanol/water (3.4 mL). Lithium hydroxide monohydrate (0.143 g, 3.41 mmole) was added and the mixture was stirred at RT for 3 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was dissolved in THF (3.4 mL). CDI (0.291 g, 1.79 mmole) was added portionwise (gas evolution was observed) and the solution was stirred at RT for 45 minutes. L-Serine methyl ester hydrochloride (0.341 g, 2.19 mmole) was added followed by N,N-diisopropylethylamine (0.390 mL, 2.24 mmole) and the mixture was stirred at RT for 18 hours. Water (25 mL) was added and the mixture was extracted with ethyl acetate (3×10 mL). The combined organics were dried over magnesium sulfate, filtered and concentrated. The residue was dissolved in DCM (3.4 mL), then imidazole (0.129 g, 1.89 mmole) and tert-butyldimethylchlorosilane (0.280 g, 1.86 mmole) were added. The mixture was stirred at RT for 3 hours and water (25 mL) was added. The mixture was extracted with DCM (3×10 mL) and the combined organics were dried over magnesium sulfate, filtered and concentrated. The crude residue was purified by silica gel chromatography to provide tert-butyl (R)-4-(4-(((S)-3-((tert-butyldimethylsilyl)oxy)-1-methoxy-1-oxopropan-2-yl)carbamoyl)thiazol-2-yl)-2-methylpiperazine-1-carboxylate as a yellow oil (0.797 g, 86%).

¹H NMR (400 MHz, CDCl₃): δ 7.94 (d, J=8.7 Hz, 1H), 7.39 (s, 1H), 4.82-4.74 (m, 1H), 4.38 (s, 1H), 4.22-4.13 (m, 2H), 4.00-3.85 (m, 4H), 3.77 (d, J=5.0 Hz, 4H), 3.68 (dt, J=12.8, 1.8 Hz, 1H), 3.30 (dd, J=12.8, 4.1 Hz, 1H), 3.23 (ddd, J=13.3, 11.9, 3.5 Hz, 1H), 3.07 (td, J=12.1, 3.7 Hz, 1H), 1.49 (s, 9H), 1.26-1.21 (m, 3H), 0.88 (s, 9H), 0.05 (s, 3H), 0.03 (s, 3H).

### Tert-butyl (R)-4-(4-(((6S,9S)-9-(methoxycarbonyl)-2,2,3,3-tetramethyl-7,12-dioxo-4,11-dioxa-8-aza-3-silatridecan-6-yl)carbamoyl)thiazol-2-yl)-2-methylpiperazine-1-carboxylate (60.3)

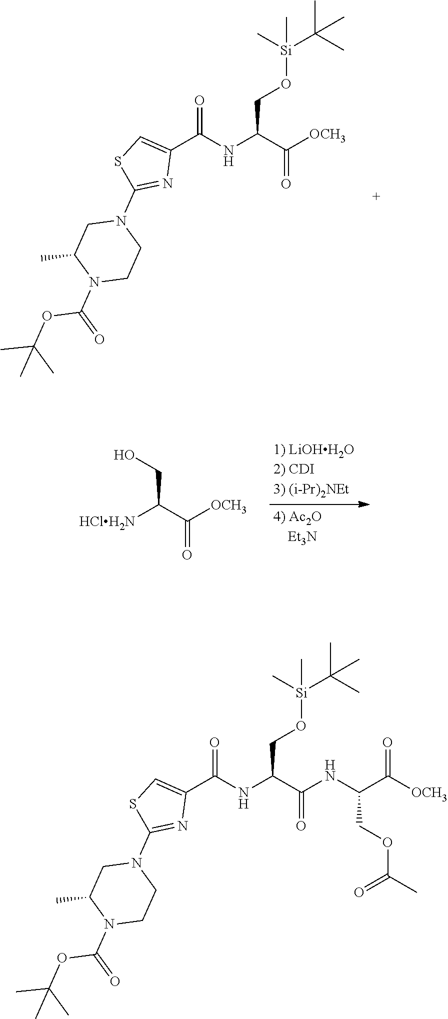

Tert-butyl (R)-4-(4-(((S)-3-((tert-butyldimethylsilyl)oxy)-1-methoxy-1-oxopropan-2-yl)carbamoyl)thiazol-2-yl)-2-methylpiperazine-1-carboxylate (0.797 g, 1.47 mmole) was dissolved in 4/1/1 THF/methanol/water (3 mL). Lithium hydroxide monohydrate (0.131 g, 3.12 mmole) was added and the mixture was stirred at RT for 3 hours. Water (25 mL) was added and the solution was treated with 1N aq. HCl to pH=4. The mixture was extracted with ethyl acetate (3×10 mL) and the combined organics were dried over magnesium sulfate, filtered and concentrated. The residue was suspended in THF (3 mL). CDI (0.251 g, 1.55 mmole) was added portionwise (gas evolution was observed) and the solution was stirred at RT for 45 minutes. L-Serine methyl ester hydrochloride (0.296 g, 1.90 mmole) was added followed by N,N-diisopropylethylamine (0.330 mL, 1.89 mmole) and the mixture was stirred at RT for 18 hours. Water (25 mL) was added and the mixture was extracted with ethyl acetate (3×10 mL). The combined organics were dried over magnesium sulfate, filtered and concentrated. The residue was dissolved in DCM (3 mL), then triethylamine (0.230 mL, 1.64 mmole) and acetic anhydride (0.60 mL, 1.69 mmole) were added. The mixture was stirred at RT for 3 hours and water (25 mL) was added. The mixture was extracted with DCM (3×10 mL) and the combined organics were dried over magnesium sulfate, filtered and concentrated. The crude residue was purified by silica gel chromatography to provide tert-butyl (R)-4-(4-(((6S,9S)-9-(methoxycarbonyl)-2,2,3,3-tetramethyl-7,12-dioxo-4,11-dioxa-8-aza-3-silatridecan-6-yl)carbamoyl)thiazol-2-yl)-2-methylpiperazine-1-carboxylate as a thick, pale yellow oil (0.463 g, 47%).

¹H NMR (400 MHz, CDCl₃): δ 7.97 (d, J=7.0 Hz, 1H), 7.44 (d, J=7.7 Hz, 1H), 7.41 (s, 1H), 4.88 (dt, J=7.7, 3.8 Hz, 1H), 4.58 (tt, J=6.7, 3.3 Hz, 1H), 4.48 (dd, J=11.3, 3.9 Hz, 1H), 4.39 (t, J=5.8 Hz, 1H), 4.32 (dd, J=11.4, 3.7 Hz, 1H), 4.19 (dd, J=9.8, 3.6 Hz, 1H), 3.93 (dd, J=18.1, 13.4 Hz, 2H), 3.82 (d, J=14.0 Hz, 1H), 3.76 (d, J=3.4 Hz, 3H), 3.75-3.71 (m, 1H), 3.68 (dd, J=13.2, 6.2 Hz, 1H), 3.35-3.18 (m, 2H), 3.07 (tt, J=12.3, 6.6 Hz, 1H), 2.03 (d, J=1.2 Hz, 3H), 1.49 (s, 9H), 1.25-1.20 (m, 4H), 0.93 (s, 9H), 0.15 (s, 3H), 0.13 (s, 3H).

### Tert-butyl (R)-4-(4-((3-((3-methoxy-3-oxoprop-1-en-2-yl)amino)-3-oxoprop-1-en-2-yl)carbamoyl)thiazol-2-yl)-2-methylpiperazine-1-carboxylate (60)

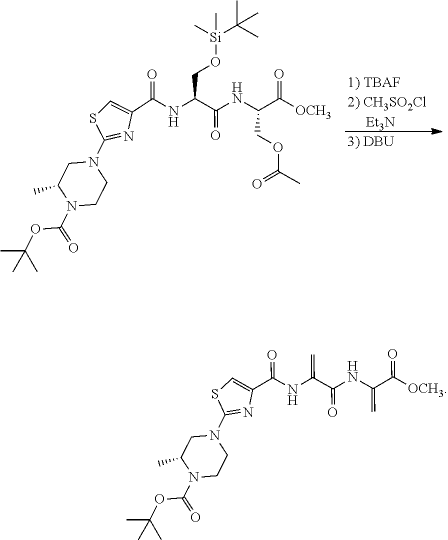 [SMILES: COC(=O)[C@H](COC(C)=O)NC(=O)[C@H](CO[Si](C)(C)C(C)(C)C)NC(=O)c1csc(N2CCN(C(=O)OC(C)(C)C)[C@H](C)C2)n1 ; C=C(NC(=O)c1csc(N2CCN(C(=O)OC(C)(C)C)[C@H](C)C2)n1)C(=O)NC(=C)C(=O)OC]

Tert-butyl (R)-4-(4-(((6S,9S)-9-(methoxycarbonyl)-2,2,3,3-tetramethyl-7,12-dioxo-4,11-dioxa-8-aza-3-silatridecan-6-yl)carbamoyl)thiazol-2-yl)-2-methylpiperazine-1-carboxylate (0.463 g, 0.689 mmole) was dissolved in THF (2 mL). TBAF (1M solution in THF, 1.70 mL, 1.70 mmole) was added and the solution was stirred at RT for 24 hours. Water (25 mL) and sat. aq. sodium chloride (15 mL) were added and the mixture was extracted with ethyl acetate (3×10 mL). The combined organics were dried over magnesium sulfate, filtered and concentrated. The residue was dissolved in DCM (2 mL) and cooled to 0° C. Triethylamine (0.150 mL, 1.07 mmole) and methanesulfonyl chloride (0.080 mL, 1.03 mmole) were added and the mixture was stirred at 0° for 60 minutes. Water (25 mL) was added and the mixture was extracted with DCM (3×10 mL). The combined organics were dried over magnesium sulfate, filtered and concentrated. The residue was dissolved in THF (2 mL) and cooled to 0° C. DBU (0.155 mL, 1.04 mmole) was added and the mixture was stirred at 0° for 60 minutes. Water (25 mL) and sat. aq. sodium chloride (15 mL) were added and the mixture was extracted with ethyl acetate (3×10 mL). The combined organics were dried over magnesium sulfate, filtered and concentrated. The crude residue was purified by silica gel chromatography to provide tert-butyl (R)-4-(4-((3-((3-methoxy-3-oxoprop-1-en-2-yl)amino)-3-oxoprop-1-en-2-yl)carbamoyl)thiazol-2-yl)-2-methylpiperazine-1-carboxylate as a white solid (0.150 g, 45%).

¹H NMR (400 MHz, CDCl3): δ 9.72 (s, 1H), 8.51 (s, 1H), 7.44 (s, 1H), 6.72 (d, J=2.1 Hz, 1H), 6.67 (s, 1H), 6.01 (d, J=1.2 Hz, 1H), 5.44 (t, J=1.9 Hz, 1H), 4.41 (s, 1H), 3.98 (ddd, J=12.5, 7.9, 5.5 Hz, 2H), 3.89 (s, 3H), 3.70 (dt, J=12.8, 1.8 Hz, 1H), 3.32 (dd, J=12.8, 4.0 Hz, 1H), 3.24 (ddd, J=13.6, 11.9, 3.6 Hz, 1H), 3.11 (td, J=12.2, 3.8 Hz, 1H), 1.49 (s, 9H), 1.25 (dd, J=6.9, 1.5 Hz, 3H).

The following Compounds 61-65 are synthesized with procedures similar to those provided above.

### Compound 61: Methyl 2-(2-(2-(4-((3-methoxypropyl)carbamoyl)piperidin-1-yl)thiazole-4-carboxamido)acrylamido)acrylate

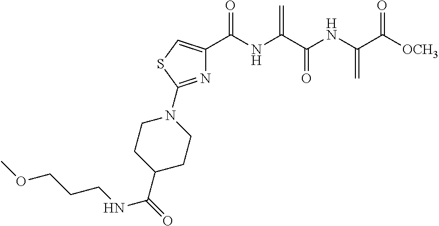 [SMILES: C=C(NC(=O)c1csc(N2CCC(C(=O)NCCCOC)CC2)n1)C(=O)NC(=C)C(=O)OC]

### Compound 62: Methyl 2-(2-(2-(4-(2-((3-methoxypropyl)amino)-2-oxoethyl)piperidin-1-yl)thiazole-4-carboxamido)acrylamido)acrylate

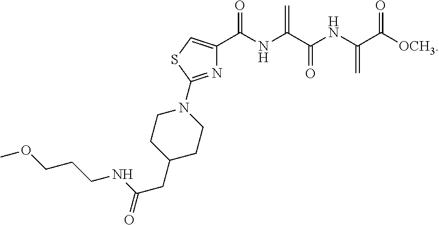 [SMILES: C=C(NC(=O)c1csc(N2CCC(CC(=O)NCCCOC)CC2)n1)C(=O)NC(=C)C(=O)OC]

### Compound 63: Methyl 2-(2-(2-(4-(3-methoxypropyl)-3-oxopiperazin-1-yl)thiazole-4-carboxamido)acrylamido)acrylate

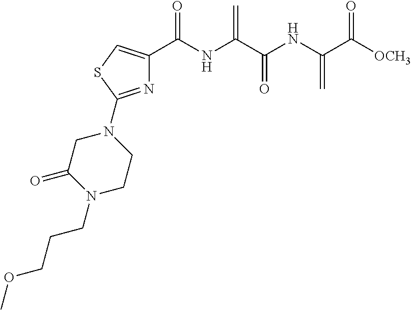 [SMILES: C=C(NC(=O)c1csc(N2CCN(CCCOC)C(=O)C2)n1)C(=O)NC(=C)C(=O)OC]

### Compound 64: Methyl (S)-2-(2-(2-(3-(tetrahydro-2H-pyran-4-carboxamido)pyrrolidin-1-yl)thiazole-4-carboxamido)acrylamido)acrylate

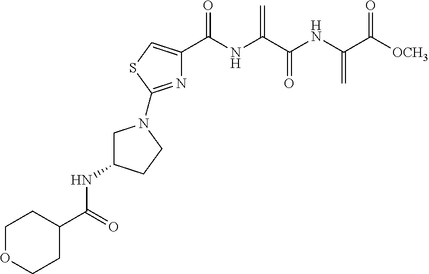 [SMILES: C=C(NC(=O)c1csc(N2CC[C@H](NC(=O)C3CCOCC3)C2)n1)C(=O)NC(=C)C(=O)OC]

### Compound 65: Methyl (R)-2-(2-(2-(3-(tetrahydro-2H-pyran-4-carboxamido)pyrrolidin-1-yl)thiazole-4-carboxamido)acrylamido)acrylate

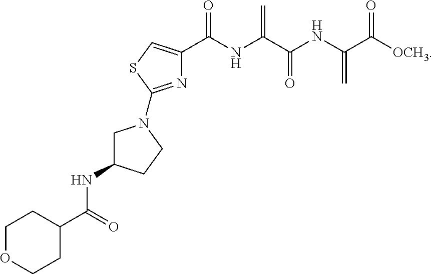 [SMILES: C=C(NC(=O)c1csc(N2CC[C@@H](NC(=O)C3CCOCC3)C2)n1)C(=O)NC(=C)C(=O)OC]

### Assay Description and Protocols

### Description of Activity Assays to Measure PRX3 Inhibition

The compounds described herein generally react with two essential cysteine residues in the mitochondrial peroxidase PRX3, leading to the formation of a non-reducible, covalent crosslink of two PRX3 monomers. This crosslink inactivates PRX3. Increased PRX3 crosslink is associated with mitochondrial stress, increased cell death in cell models of malignant mesothelioma and is associated with decreased tumor volume in a mouse model. Cunniff et al. (2015) PloS one 10, e0127310.

PRX3 uses an essential reduced cysteine residue to reduce hydrogen peroxide. During this process, PRX3 becomes oxidized forming a reversible disulfide bond that links two PRX3 monomers. In the cell, this disulfide can be reduced by the combined activity of thioredoxin 2 (TRX2), thioredoxin reductase 2, and NADPH. The disulfide can also be reduced by small molecule reductants such as dithiothreitol (DTT). In contrast, the compound-crosslink with PRX3 is irreversible and cannot be broken by the addition of reductants.

To test the activity of compounds, two complimentary assays were used as described below. The Biochemical PRX3 Inhibition Assay tests the ability of each compound to crosslink PRX3 in a simple, in vitro system. In the Cellular Activity Assay, we are testing the ability of compounds to kill SK-OV-3 ovarian cancer cells.

### Biochemical PRX3 Inhibition Assay

This assay tests the ability of each compound to form a covalent adduct with PRX3. The assay is performed as described in Nelson et al. (2021) Antioxidants (Basel) 10, 150; and Cunniff et al. (2015)PloS one 10, e0127310, and follows the appearance of non-reducible PRX3 crosslinks by SDS PAGE gel electrophoresis and follows the appearance of single PRX3 adducts by mass spectrometry. For this assay, purified human PRX3, hydrogen peroxide (the PRX3 substrate) and all the components required to enable PRX3 to catalytically cycle (thioredoxin-thioredoxin reductase-NAPDH system) are included (details below).

The biochemical PRX3 Inhibition assay contains 100 μM PRX3, 50 μM human TRX2, 0.5 μM mouse thioredoxin reductase, and a NADPH regenerating system composed of 3.2 mM glucose 6-phosphate, 3.2 U/ml glucose 6-phosphate dehydrogenase and 0.4 mM NADPH. Samples are incubated for 1-2 hr at 37° C. with either 0.2 mM TS (positive control), compounds or an equivalent volume of DMSO (negative control). During this incubation, hydrogen peroxide is added to induce turnover of PRX3. Reactions are stopped by the addition of a buffer containing 100 mM dithiothreitol (to break disulfide bonds) and SDS (detergent to denature proteins). NADPH, Glucose 6-Phosphate, and glucose 6-phosphate dehydrogenase were purchased from Sigma Aldrich. PRX3, thioredoxin, and thioredoxin reductase were all purified to >98% purity in the Lowther laboratory according to protocols referenced in Nelson et al. (2021) Antioxidants (Basel) 10, 150; and Cunniff et al. (2015) PloS one 10, e0127310.

To measure the amount of PRX3 crosslink, proteins in the reaction were separated by SDS-polyacrylamide gel electrophoresis and stained for total protein using GelCode Blue (Life Technologies). The amount of unmodified PRX3 and TS-PRX3 crosslink was measured by densiometric analysis of the signal for the PRX3 band running at the MW of a PRX3 crosslink (˜46 kDa) compared to the PRX3 signal at the MW of the un-modified PRX3 (˜23 kD).

To measure covalent adduct formation on a single PRX3 monomer, each reaction was exchanged into a mass spectrometry compatible buffer containing 40 mM ammonium citrate, pH 8.3 made in HPLC water. Sample was mixed 1:1 with a matrix solution containing 30 mg/mL sinapinic acid in 70% (vol/vol) acetonitrile, 0.2% formic acid and spotted to onto the sample plate. PRX3 mass was measured by MALDI-TOF MS analysis on a Bruker Daltonics MALDI-TOF MS spectrometer. Spectra were analyzed in FLEXAnalysis Software. The intensity of the reduced PRX3 peak (SH) and intensity of the analog adduct peak was determined and corrected for background signal at the adduct peak in the DMSO control. The fraction of single analog adduct in the monomer peak was determined by dividing the intensity of the adduct peak by the summed intensity of the SH and adduct peaks.

### Cellular Activity Assay

The Cellular Activity Assay measures the EC₅₀ of each compound in human SK-OV-3 ovarian cancer cells TS to be taken into the cell, transported to the mitochondria, and crosslink PRX3.

The cytotoxicity of compounds was measured as described in Nelson et al. (2021) Antioxidants (Basel) 10, 150; Cunniff et al. (2015) PloS one 10, e0127310; and Newick et al. (2012)PloS one 7, e39404. SK-OV-3 is an adherent, epithelial, adenocarcinoma cell line obtained from ATCC (ref #: HTB-77). SK-OV-3 cells are resistant to the commonly used chemotherapeutics, cis-platinum and doxorubicin. For this PRX3 crosslinking assay, SK-OV-3 cells were plated in a 96-well plate. After 24 h recovery, the cells were treated for 48 hr with multiple concentrations of each compound ranging from 0.1-100 μM.

Cells were washed with PBS to remove dead cells and the remaining live cells were fixed with 3.0% formaldehyde and stained with 0.1% crystal violet. The amount of crystal violet was determined by reading the absorbance at 540 nm (crystal violet dye dissolved in 100% methanol) using a plate reader. The signal for crystal violet dye is proportional to the biomass of remaining live cells. GraphPad Prism9 software was used to calculate the effective inhibitory concentration (EC₅₀) of test compounds. Results for each compound, are normalized to the amount of cells in control wells treated with the equivalent concentration of DMSO (negative control). TS or Compound 4 were included in each set of assays as a positive control.

### Solubility Assay

A 20 mM stock solution was made for each compound in DMSO. To measure solubility, 0.03-0.05 mL of the stock was added to 0.27 mL 20 mM HEPES, 100 mM NaCl, pH 7.5. The solution was mixed and rotated 18-24 hours at ambient temperature. The next day, solutions were centrifuged for 10 min at 20,000×g at 24° C. to remove insoluble, solid material. Next, 100 μL of the supernatant containing the soluble compound was added to 900 μL DMSO and High Performance Liquid Chromatography was performed as described in Table 1 and either HPLC gradient 1 (Table 2) or HPLC gradient 2 (Table 3). For each analog, a 200 μM standard and a 20 μM standard were prepared and run in parallel (standards were dissolved completely in 100% DMSO). The amount of compound in the experimental sample was calculated by comparing the area under the curve for compound peak to the area for the closest standard and correcting for dilution.

TABLE 1
HPLC Instrument Parameters for Solubility Assay

| Instrument | Agilent 1260 Infinity II HPLC System | |
|---|---|---|
| Software to | OpenLab CDS Chemstation Edition Software for | |
| Process data | LC and LC/MS Systems. Rev. C.01.09 | |
| | (144) from Agilent Technologies | |
| Column | 100 mM Poroshell 120EC-C18 4.6 × | |
| | 100 mm × 2.7 μm | |
| | Agilent Product #: 695975-9027. | |
| | Serial #: USCFS26515 | |
| | Batch #: B19136 | |
| Buffer A | Water 0.1% Trifluoroacetic acid (TFA) | |
| Buffer B | Acetonitrile 0.1% TFA | |
| Flow Rate | 0.8 | mL/min |
| wavelength | 254 | nm |
| Col Temp | 30° | C. |
| Autosampler Temp | 23° | C. |
| Seal wash solvent | 50% Acetonitirle: 50% Water | |
| Needle wash solvent | 50% Acetonitrile: 50% Water | |
| Diluent | DMSO | |
| Sample Preparation | 0.1 mL sample to 0.9 mL DMSO | |
| Dilution Factor | 10 | |
| Injection vol | 10 | μl |
| Time | 14.1 | min |

TABLE 2
HPLC Gradient Program 1

| Time (min) | % H2O | % CH3CN | ml/min | max pressure |
|---|---|---|---|---|
| 0 | 70 | 30 | 1.0 | 400 |
| 2 | 0 | 98 | 1.0 | 400 |
| 8 | 0 | 98 | 1.0 | 400 |
| 10 | 70 | 30 | 1.0 | 400 |
| 11 | 70 | 30 | 1.0 | 400 |
| 14 | 70 | 30 | 1.0 | 400 |

TABLE 3
Alternate HPLC Gradient 2

| Time (min) | % H2O | % CH3CN | ml/min | max pressure |
|---|---|---|---|---|
| 0 | 70 | 30 | 1.0 | 400 |
| 2 | 0 | 98 | 1.0 | 400 |
| 8 | 0 | 98 | 1.0 | 400 |
| 10 | 70 | 30 | 1.0 | 400 |
| 11 | 70 | 30 | 1.0 | 400 |
| 14 | 70 | 30 | 1.0 | 400 |

### Antimicrobial Activity Assay

To determine the minimal inhibitory concentration (MIC) of TS with Enterococcus hirae (ATCC #10541), a frozen stock of E. hirae was streaked onto a plate containing BHI growth media (7.8 g/L brain extract, 2.0 g/L dextrose, 2.5 g/L disodium phosphate, 9.7 g/L heart extract, 10 g/L proteose peptone, 5 g/L sodium chloride supplemented with 0.01% (v/v) polysorbate 80, 10 g/mL dextrose, and 10 g/L agar. A single colony of E. hirae was used to inoculate 10 mL of media 41 (described in USP37<81>: 9 g/L pancreatic digest of casein, 5 g/L yeast extract, 20 g/L dextrose, 10 g/L sodium citrate, 1 g/L monobasic potassium phosphate, and 1 g/L dibasic potassium phosphate and grown overnight at 37° C. The overnight growth was then diluted into growth media to make a single solution of E. hirae with a final optical density at 600 nm=0.02 AU. A 20 mM stock solution of Thiostrepton in DMSO was made and used to make multiple dilutions of TS in DMSO. Each TS dilution (0.1 mL) was then added to a test tube containing 10 mL of the diluted E. hirae and grown at 37° C. overnight while shaking at 180 rpm. The next morning, each tube was visually inspected to determine if the cells grew. Final concentrations tested were 0.120, 0.090, 0.075, 0.060, 0.045, 0.030, and 0.015 μM TS. Complete growth was observed at TS concentrations ≤0.030 μM TS and no growth was observed at all TS concentrations ≥0.045 μM. Three replicates were made on three separate days (n=9). No intermediate growth was observed, and all replicates showed the same growth profile. For TS analogs, the dilute E. hirae was treated with 20 μM final concentration of each analog (three replicates each). No growth inhibition was observed for any analog. DMSO was used as a positive control (complete cell growth) and 20 μM thiostrepton was used as a negative control.

### Aqueous Stability Assay

To measure aqueous stability after 24 hours, samples were prepared and analyzed by HPLC as described for the Solubility Assay. % Purity was calculated for the control sample (compound in DMSO) and the assay sample (compound in aqueous buffer) according to the following equations:

$$\% Purity = \frac{Area Compound Peak}{Total Area of all peaks} \times 100$$

Diluent peaks and solvent peaks are excluded from the calculation. Relative stability was calculated by dividing the Purity of the Assay Sample by the Purity of the Standard sample.

### Assay Results

TABLE 4
Key

| | | | PRX3 Inhibition | | | | | |
|---|---|---|---|---|---|---|---|---|
| | | | (Crosslink + | | | | | |
| | EC50 | | Mono-adduct) | Solubility | | Stability | Antimicrobial | |
| + | <1 | μM | | | | | | |
| +++ | 1-10 | μM | >90% | >1 | mM | | 0.02-0.2 | μM |
| ++ | 10-25 | μM | 50-90% | 0.1-1 | mM | >0.95 after | 0.2-2 | μM |
| | | | | | | 24 hours | | |
| + | | 25-100 μM | 5-50% | <0.1 | mM | >0.90 after | 2-20 | μM |

| | | | | | |
|---|---|---|---|---|---|
| | | | 24 hours | | |
| − | >100 μM | polymerized | <0.90 | >20 | μM |

| | |
|---|---|
| N.D. | not determined |

Assay Results for Each Compound

| | | PRX3 | | Aqueous | Antimicrobial |
|---|---|---|---|---|---|
| Compound | EC₅₀ | Inhibition | Solubility | Stability | Activity |
| 1 | +++ | ++ | ++ | − | − |
| 2 | +++ | ++ | + | ++ | − |
| 3 | ++ | ++ | ++ | − | − |
| 4 | +++ | ++ | + | ++ | − |
| 5 | +++ | +++ | + | ++ | − |
| 6 | +++ | +++ | + | − | − |
| 7 | +++ | ++ | + | ++ | − |
| 8 | +++ | +++ | + | ++ | − |
| 9 | +++ | +++ | + | − | − |
| 10 | +++ | +++ | + | + | − |
| 11 | +++ | +++ | + | ++ | − |
| 12 | +++ | +++ | + | ++ | − |
| 13 | +++ | +++ | ++ | ++ | − |
| 14 | + | +++ | + | − | − |
| 15 | +++ | +++ | + | − | − |
| 16 | ++ | +++ | + | − | − |
| 17 | ++ | +++ | ++ | ++ | − |
| 18 | + | + | ++ | ++ | − |
| 19 | +++ | +++ | + | ++ | − |
| 20 | +++ | +++ | + | ++ | − |
| 21 | +++ | +++ | + | ++ | − |
| 22 | +++ | +++ | + | ++ | − |
| 23 | +++ | +++ | + | ++ | − |
| 24 | +++ | ++ | + | ++ | − |
| 25 | ++ | ++ | + | ++ | − |
| 26 | +++ | ++ | ++ | ++ | − |
| 27 | +++ | +++ | + | − | − |
| 28 | +++ | +++ | + | − | − |
| 29 | +++ | +++ | ++ | ++ | − |
| 30 | + | + | + | ++ | − |
| 31 | + | + | +++ | − | − |
| 32 | + | ++ | ++ | − | − |
| 33 | + | +++ | +++ | − | − |
| 34 | +++ | +++ | +++ | ++ | − |
| 35 | +++ | +++ | ++ | − | N.D. |
| 36 | +++ | +++ | + | − | N.D. |
| 37 | +++ | + | + | − | N.D. |
| 38 | +++ | ++ | + | + | N.D. |
| 39 | +++ | ++ | + | ++ | N.D. |
| 40 | ++ | +++ | ++ | + | N.D. |
| 41 | ++ | +++ | +++ | ++ | N.D. |
| 42 | +++ | +++ | + | ++ | N.D. |
| 43 | +++ | +++ | + | ++ | N.D. |
| 44 | +++ | +++ | + | ++ | N.D. |
| 45 | +++ | ++ | + | ++ | N.D. |
| 46 | +++ | +++ | ++ | ++ | N.D. |
| 47 | + | ++ | +++ | + | N.D. |
| 48 | +++ | ++ | + | ++ | N.D. |
| 49 | +++ | +++ | + | ++ | N.D. |
| 50 | +++ | + | + | ++ | N.D. |
| 51 | ++ | +++ | + | ++ | N.D. |
| 52 | ++ | +++ | + | ++ | N.D. |
| 53 | ++ | +++ | + | ++ | N.D. |
| 54 | + | ++ | + | ++ | N.D. |
| 55 | ++ | ++ | + | ++ | N.D. |
| 56 | +++ | ++ | + | ++ | N.D. |
| 57 | +++ | +++ | + | ++ | N.D. |
| 58 | N.D. | +++ | ++ | + | N.D. |
| 59 | N.D. | +++ | ++ | ++ | N.D. |
| 60 | N.D. | +++ | + | ++ | N.D. |

### REFERENCES

1. Cairns, R. A., Harris, I. S., and Mak, T. W. (2011) Regulation of cancer cell metabolism. Nat Rev Cancer 11, 85-95
2. Weinberg, F., and Chandel, N. S. (2009) Reactive oxygen species-dependent signaling regulates cancer. Cell Mol Life Sci 66, 3663-3673
3. Weinberg, F., and Chandel, N. S. (2009) Mitochondrial metabolism and cancer. Ann N Y Acad Sci 1177, 66-73
4. Weinberg, F., Hamanaka, R., Wheaton, W. W., Weinberg, S., Joseph, J., Lopez, M., Kalyanaraman, B., Mutlu, G. M., Budinger, G. R., and Chandel, N. S. (2010) Mitochondrial metabolism and ROS generation are essential for Kras-mediated tumorigenicity. Proc Natl Acad Sci USA 107, 8788-8793
5. Park, H. J., Carr, J. R., Wang, Z., Nogueira, V., Hay, N., Tyner, A. L., Lau, L. F., Costa, R. H., and Raychaudhuri, P. (2009) FoxM1, a critical regulator of oxidative stress during oncogenesis. EMBO J 28, 2908-2918
6. Nonn, L., Berggren, M., and Powis, G. (2003) Increased expression of mitochondrial peroxiredoxin-3 (thioredoxin peroxidase-2) protects cancer cells against hypoxia and drug-induced hydrogen peroxide-dependent apoptosis. Mol Cancer Res 1, 682-689
7. Cox, A. G., Winterbourn, C. C., and Hampton, M. B. (2009) Mitochondrial peroxiredoxin involvement in antioxidant defence and redox signalling. Biochem J 425, 313-325
8. Tang, Z., Kang, B., Li, C., Chen, T., and Zhang, Z. (2019) GEPIA2: an enhanced web server for large-scale expression profiling and interactive analysis. Nucleic Acids Res 47, W556-W560
9. Nelson, K. J., Messier, T., Milczarek, S., Saaman, A., Beuschel, S., Gandhi, U., Heintz, N., Smalley, T. L., Lowther, W. T., and Cunniff, B. (2021) Unique Cellular and Biochemical Features of Human Mitochondrial Peroxiredoxin 3 Establish the Molecular Basis for Its Specific Reaction with Thiostrepton. Antioxidants (Basel) 10
10. Cunniff, B., Newick, K., Nelson, K. J., Wozniak, A. N., Beuschel, S., Leavitt, B., Bhave, A., Butnor, K., Koenig, A., Chouchani, E. T., James, A. M., Haynes, A. C., Lowther, W. T., Murphy, M. P., Shukla, A., and Heintz, N. H. (2015) Disabling Mitochondrial Peroxide Metabolism via Combinatorial Targeting of Peroxiredoxin 3 as an Effective Therapeutic Approach for Malignant Mesothelioma. PloS one 10, e0127310
11. Myers, C. R. (2016) Enhanced targeting of mitochondrial peroxide defense by the combined use of thiosemicarbazones and inhibitors of thioredoxin reductase. Free Radic Biol Med 91, 81-92
12. Yoshikawa, N., Kajiyama, H., Nakamura, K., Utsumi, F., Niimi, K., Mitsui, H., Sekiya, R., Suzuki, S., Shibata, K., Callen, D., and Kikkawa, F. (2016) PRIMA-1MET induces apoptosis through accumulation of intracellular reactive oxygen species irrespective of p53 status and chemo-sensitivity in epithelial ovarian cancer cells. Oncol Rep 35, 2543-2552
13. Wang, X. Y., Wang, H. J., and Li, X. Q. (2013) Peroxiredoxin III protein expression is associated with platinum resistance in epithelial ovarian cancer. Tumour Biol 34, 2275-2281
14. Li, S., Hu, X., Ye, M., and Zhu, X. (2018) The prognostic values of the peroxiredoxins family in ovarian cancer. Biosci Rep 38
15. Li, L., Shoji, W., Takano, H., Nishimura, N., Aoki, Y., Takahashi, R., Goto, S., Kaifu, T., Takai, T., and Obinata, M. (2007) Increased susceptibility of MER5 (peroxiredoxin III) knockout mice to LPS-induced oxidative stress. Biochem Biophys Res Commun 355, 715-721
16. Lee, Y. J. (2020) Knockout Mouse Models for Peroxiredoxins. Antioxidants (Basel) 9
17. Corsello, S. M., Nagari, R. T., Spangler, R. D., Rossen, J., Kocak, M., Bryan, J. G., Humeidi, R., Peck, D., Wu, X., Tang, A. A., Wang, V. M., Bender, S. A., Lemire, E., Narayan, R., Montgomery, P., Ben-David, U., Garvie, C. W., Chen, Y., Rees, M. G., Lyons, N. J., McFarland, J. M., Wong, B. T., Wang, L., Dumont, N., O'Hearn, P. J., Stefan, E., Doench, J. G., Harrington, C. N., Greulich, H., Meyerson, M., Vazquez, F., Subramanian, A., Roth, J. A., Bittker, J. A., Boehm, J. S., Mader, C. C., Tsherniak, A., and Golub, T. R. (2020) Discovering the anticancer potential of non-oncology drugs by systematic viability profiling. Nature Cancer
18. Newick, K., Cunniff, B., Preston, K., Held, P., Arbiser, J., Pass, H., Mossman, B., Shukla, A., and Heintz, N. (2012) Peroxiredoxin 3 is a redox-dependent target of thiostrepton in malignant mesothelioma cells. PloS one 7, e39404
19. Hegde, N. S., Sanders, D. A., Rodriguez, R., and Balasubramanian, S. (2011) The transcription factor FOXM1 is a cellular target of the natural product thiostrepton. Nat Chem 3, 725-731
20. Bhat, U. G., Halasi, M., and Gartel, A. L. (2009) FoxM1 is a general target for proteasome inhibitors. PloS one 4, e6593
21. Bird, K. E., Xander, C., Murcia, S., Schmalstig, A. A., Wang, X., Emanuele, M. J., Braunstein, M., and Bowers, A. A. (2020) Thiopeptides Induce Proteasome-Independent Activation of Cellular Mitophagy. ACS Chem Biol 15, 2164-2174
22. Zhang, L., Ging, N. C., Komoda, T., Hanada, T., Suzuki, T., and Watanabe, K. (2005) Antibiotic susceptibility of mammalian mitochondrial translation. FEBS Lett 579, 6423-6427
23. Harms, J. M., Wilson, D. N., Schluenzen, F., Connell, S. R., Stachelhaus, T., Zaborowska, Z., Spahn, C. M., and Fucini, P. (2008) Translational regulation via L11: molecular switches on the ribosome turned on and off by thiostrepton and micrococcin. Mol Cell 30, 26-38
24. Livnat-Levanon, N., Kevei, E., Kleifeld, O., Krutauz, D., Segref, A., Rinaldi, T., Erpapazoglou, Z., Cohen, M., Reis, N., Hoppe, T., and Glickman, M. H. (2014) Reversible 26S proteasome disassembly upon mitochondrial stress. Cell Rep 7, 1371-1380
25. Segref, A., Kevei, E., Pokrzywa, W., Schmeisser, K., Mansfeld, J., Livnat-Levanon, N., Ensenauer, R., Glickman, M. H., Ristow, M., and Hoppe, T. (2014) Pathogenesis of human mitochondrial diseases is modulated by reduced activity of the ubiquitin/proteasome system. Cell Metab 19, 642-652
26. Ayida, B. K., Simonsen, K. B., Vourloumis, D., and Hermann, T. (2005) Synthesis of dehydroalanine fragments as thiostrepton side chain mimetics. Bioorg Med Chem Lett 15, 2457-2460
27. Vaillard, V., Gonzalez, M., Perotti, J., Frau, R., and Vaillard, S. (2014) Method for the synthesis of N-alkyl-O-alkyl carbamates. RSC Advances 4, 13012-13017

The foregoing is illustrative of the present invention, and is not to be construed as limiting thereof. The invention is defined by the following claims, with equivalents of the claims to be included therein.

1. A compound of Formula I: 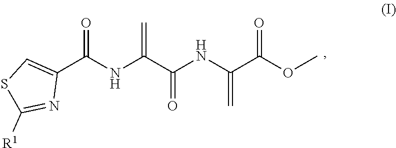
wherein R¹ is an aryl, heteroaryl, cycloalkyl or heterocycle, which aryl, heteroaryl, cycloalkyl or heterocycle is optionally substituted with one or more selected from alkyl, carboxy, carbamate, urea, amide, amino, ether, ester and halo; and
wherein when R¹ is pyridine or pyrazine, said pyridine or pyrazine is substituted with one or more selected from alkyl, carboxy, carbamate, urea, amide, amino, ether, ester and halo,
or a pharmaceutically acceptable salt thereof.

2. The compound of claim 1, wherein said aryl, heteroaryl, cycloalkyl or heterocycle, is substituted with one or more selected from alkyl, carboxy, carbamate, urea, amide, amino, ether, ester and halo.

3. The compound of claim 1, wherein said aryl, heteroaryl, cycloalkyl or heterocycle, is substituted with carbamate or amide.

4. The compound of claim 1, wherein said aryl, heteroaryl, cycloalkyl or heterocycle, is substituted with an alkylcarbamate.

5. The compound of claim 1, wherein R¹ is a group having a structure of: 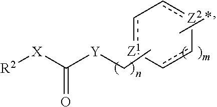
wherein:
n is 0, 1, 2 or 3;
m is 0, 1 or 2;
X is absent or is O, NR³, or CH₂;
Y is absent or is O, NR³, or CH₂;
Z¹ and Z² are each independently O, N, or C;
R² is alkyl (e.g., having from 1 to 8 carbon atoms, linear or branched), wherein said alkyl is optionally substituted (e.g., with halo, amino, ether, alkoxy, or carbamate), or heterocycle; and
R³ is H or alkyl (e.g., having from 1 to 8 carbon atoms, linear or branched),
wherein * denotes the connection of the group in the compound of Formula I,
or a pharmaceutically acceptable salt thereof.

6. The compound of claim 5, wherein Z¹ and Z² are each independently N or C.

7. The compound of claim 1, wherein the compound is selected from the group consisting of: 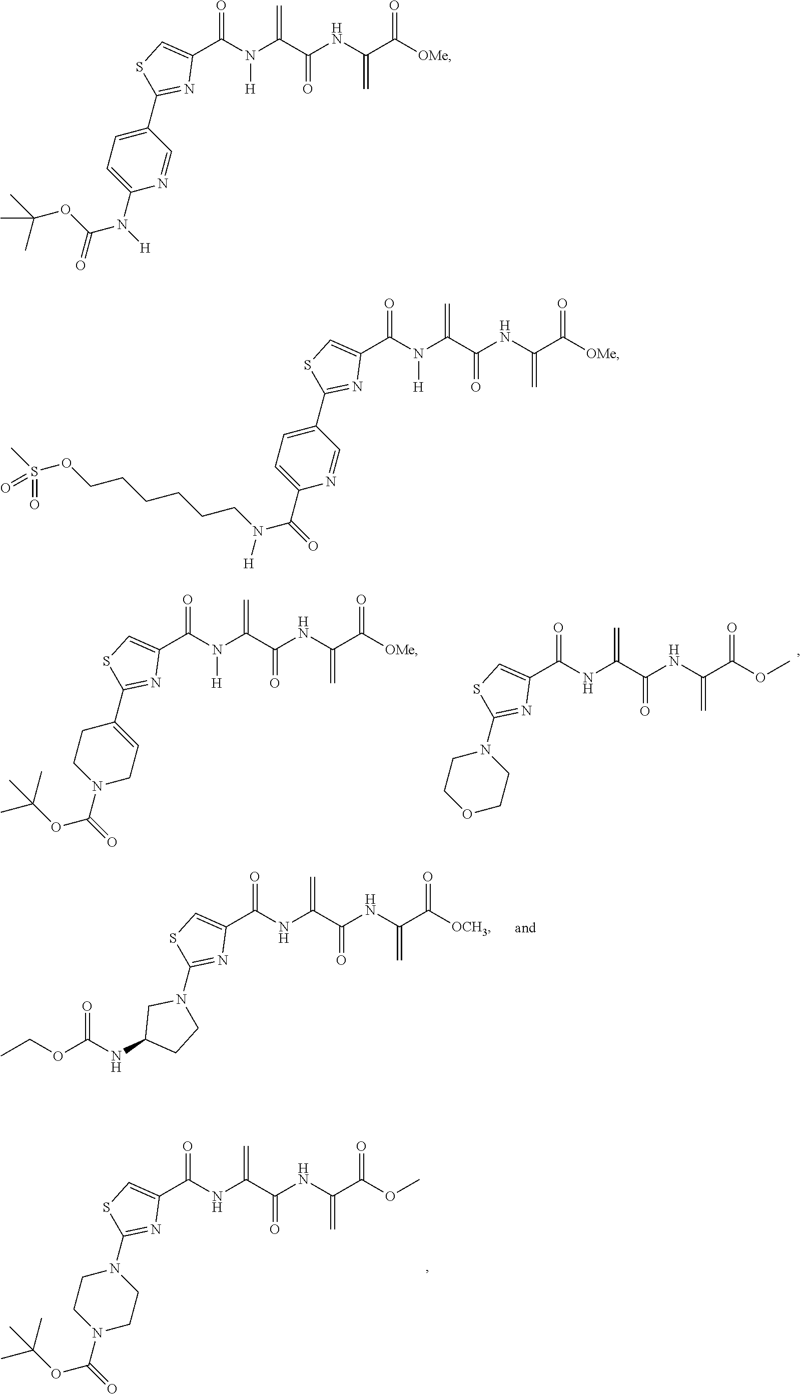
or a pharmaceutically acceptable salt thereof.

8. The compound of claim 1, wherein the compound is selected from the group consisting of: 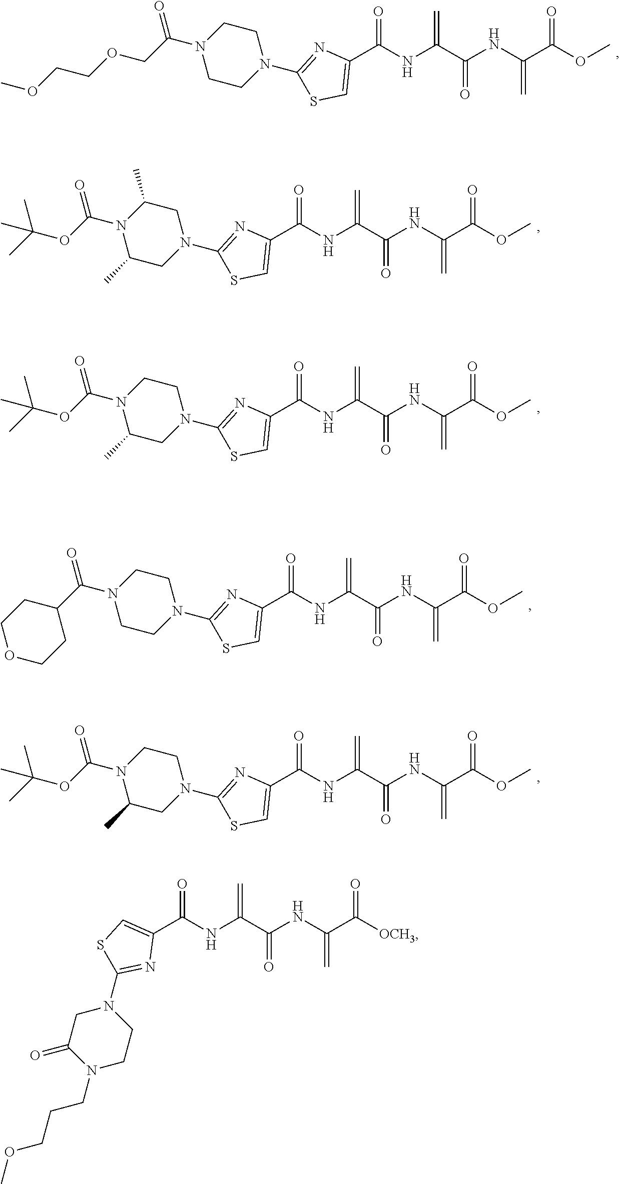 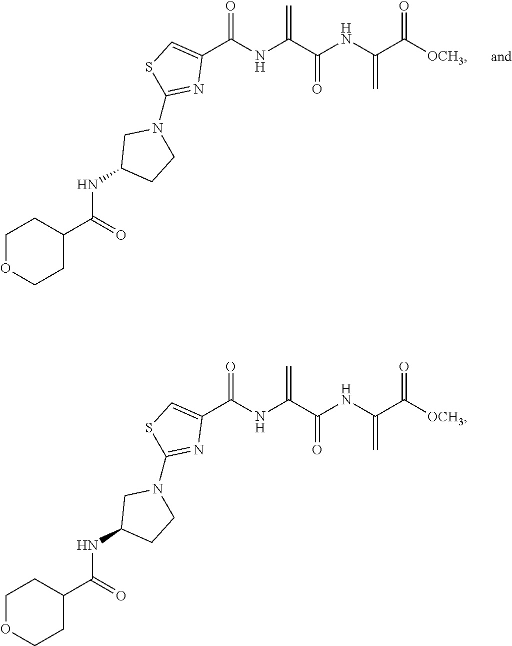 [SMILES: C=C(NC(=O)c1csc(N2CC[C@H](NC(=O)C3CCOCC3)C2)n1)C(=O)NC(=C)C(=O)OC ; C=C(NC(=O)c1csc(N2CC[C@@H](NC(=O)C3CCOCC3)C2)n1)C(=O)NC(=C)C(=O)OC]
or a pharmaceutically acceptable salt thereof.

9. The compound of claim 1, wherein R¹ is a group having a structure of: 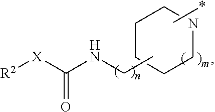
wherein:
n is 0, 1, 2 or 3;
m is 0, 1 or 2;
X is O or CH₂; and
R² is alkyl (e.g., having from 1 to 8 carbon atoms, linear or branched), wherein said alkyl is optionally substituted (e.g., with halo, amino, ether, alkoxy, or carbamate), or heterocycle; and
wherein * denotes the connection of the group in the compound of Formula I,
or a pharmaceutically acceptable salt thereof.

10. The compound of claim 1, wherein R¹ is a group having a structure of: 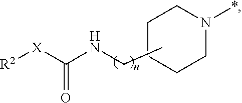
wherein:
n is 0, 1, 2 or 3;
X is O or CH₂; and
R² is alkyl (e.g., having from 1 to 8 carbon atoms, linear or branched), wherein said alkyl is optionally substituted (e.g., with halo, amino, ether, alkoxy, or carbamate), or heterocycle; and
wherein * denotes the connection of the group in the compound of Formula I,
or a pharmaceutically acceptable salt thereof.

11. The compound of claim 1, wherein the compound is selected from the group consisting of: 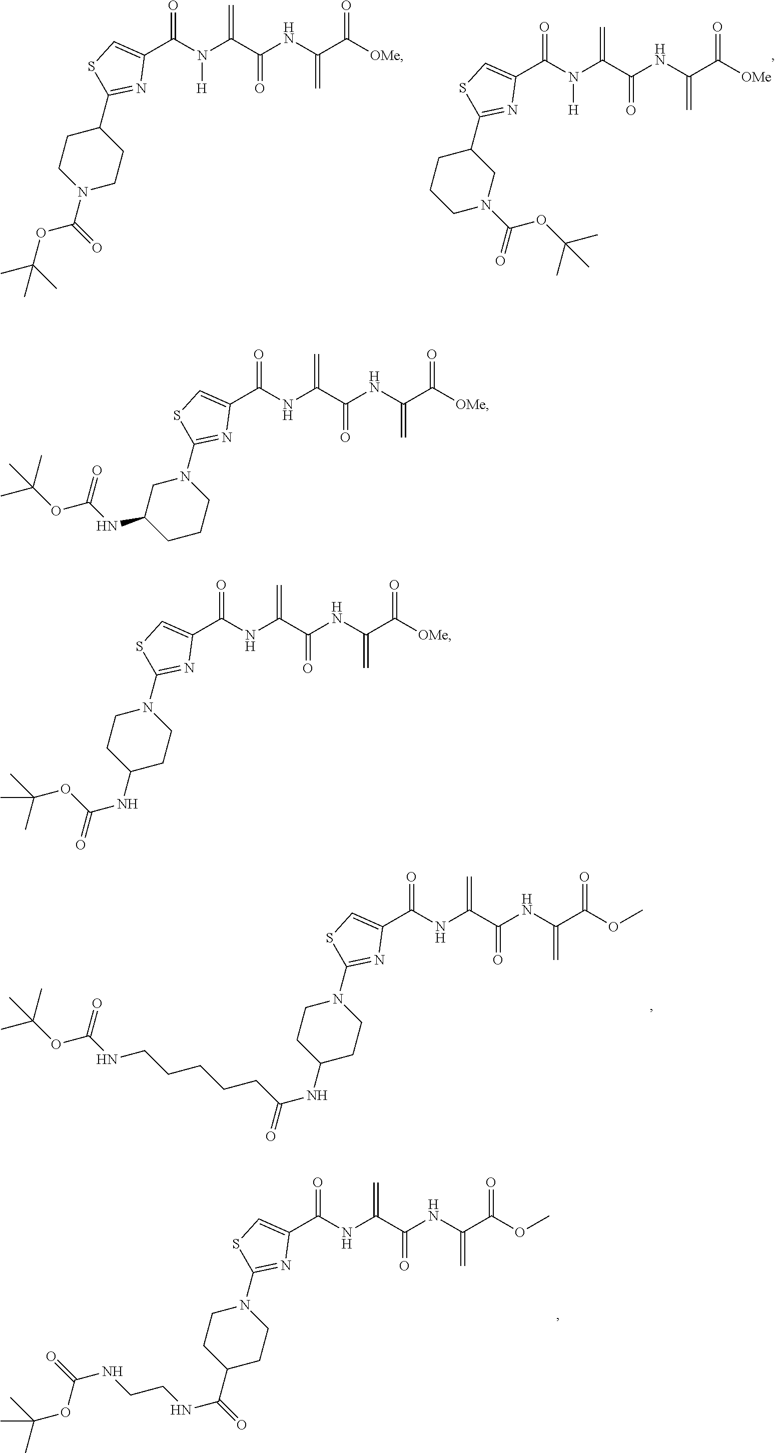 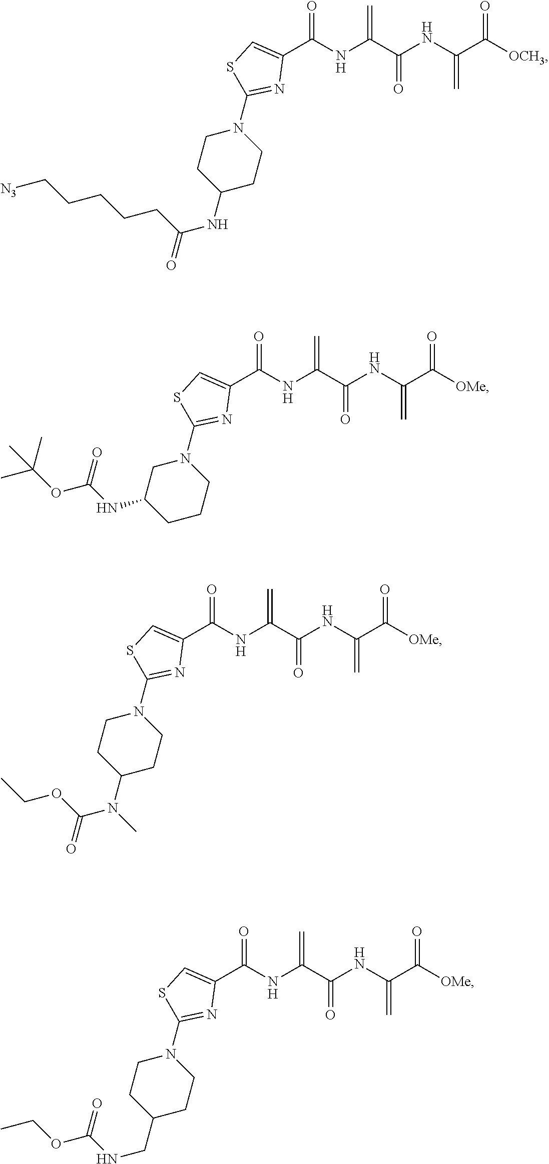 [SMILES: C=C(NC(=O)c1csc(N2CCC(NC(=O)CCCCCN=[N+]=[N-])CC2)n1)C(=O)NC(=C)C(=O)OC ; C=C(NC(=O)c1csc(N2CCC[C@H](NC(=O)OC(C)(C)C)C2)n1)C(=O)NC(=C)C(=O)OC ; C=C(NC(=O)c1csc(N2CCC(N(C)C(=O)OCC)CC2)n1)C(=O)NC(=C)C(=O)OC ; C=C(NC(=O)c1csc(N2CCC(CNC(=O)OCC)CC2)n1)C(=O)NC(=C)C(=O)OC] 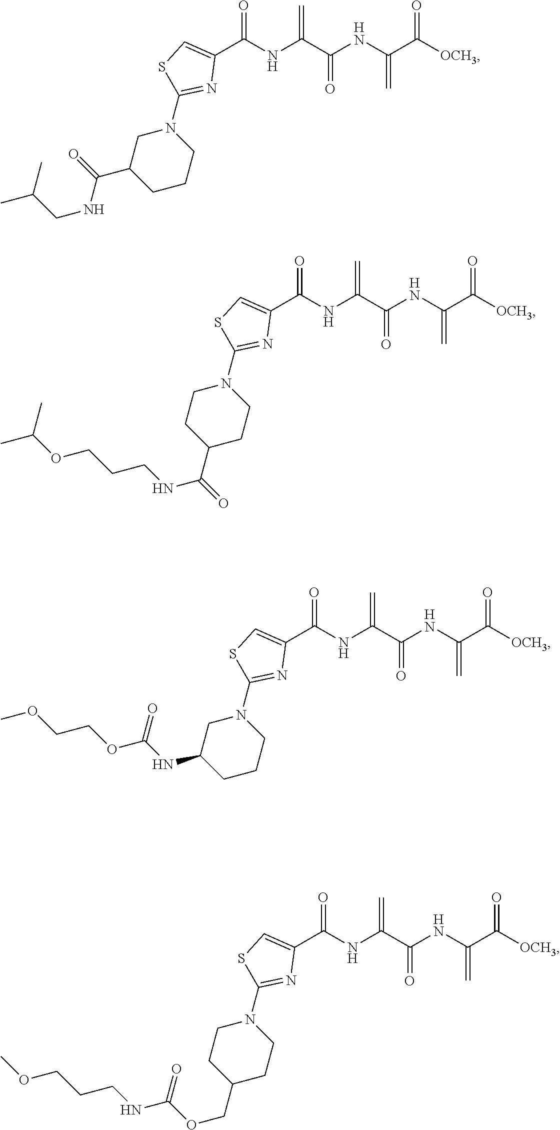 [SMILES: C=C(NC(=O)c1csc(N2CCCC(C(=O)NCC(C)C)C2)n1)C(=O)NC(=C)C(=O)OC ; C=C(NC(=O)c1csc(N2CCC(C(=O)NCCCOC(C)C)CC2)n1)C(=O)NC(=C)C(=O)OC ; C=C(NC(=O)c1csc(N2CCC[C@@H](NC(=O)OCCOC)C2)n1)C(=O)NC(=C)C(=O)OC ; C=C(NC(=O)c1csc(N2CCC(COC(=O)NCCCOC)CC2)n1)C(=O)NC(=C)C(=O)OC] 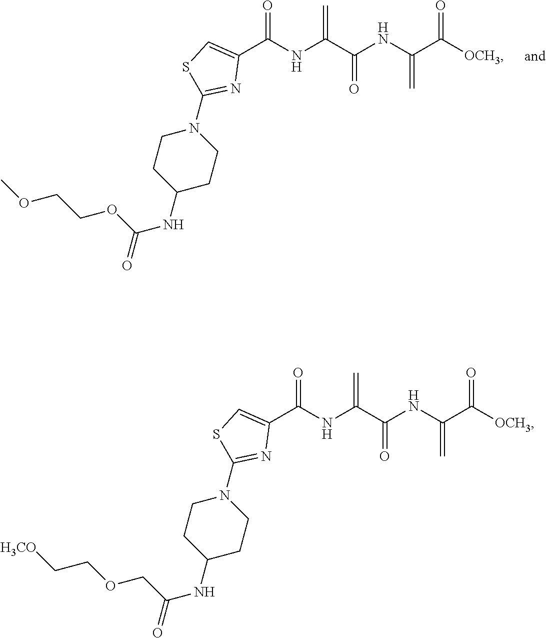 [SMILES: C=C(NC(=O)c1csc(N2CCC(NC(=O)OCCOC)CC2)n1)C(=O)NC(=C)C(=O)OC ; C=C(NC(=O)c1csc(N2CCC(NC(=O)COCCOC)CC2)n1)C(=O)NC(=C)C(=O)OC]
or a pharmaceutically acceptable salt thereof.

12. The compound of claim 1, wherein the compound is selected from the group consisting of: 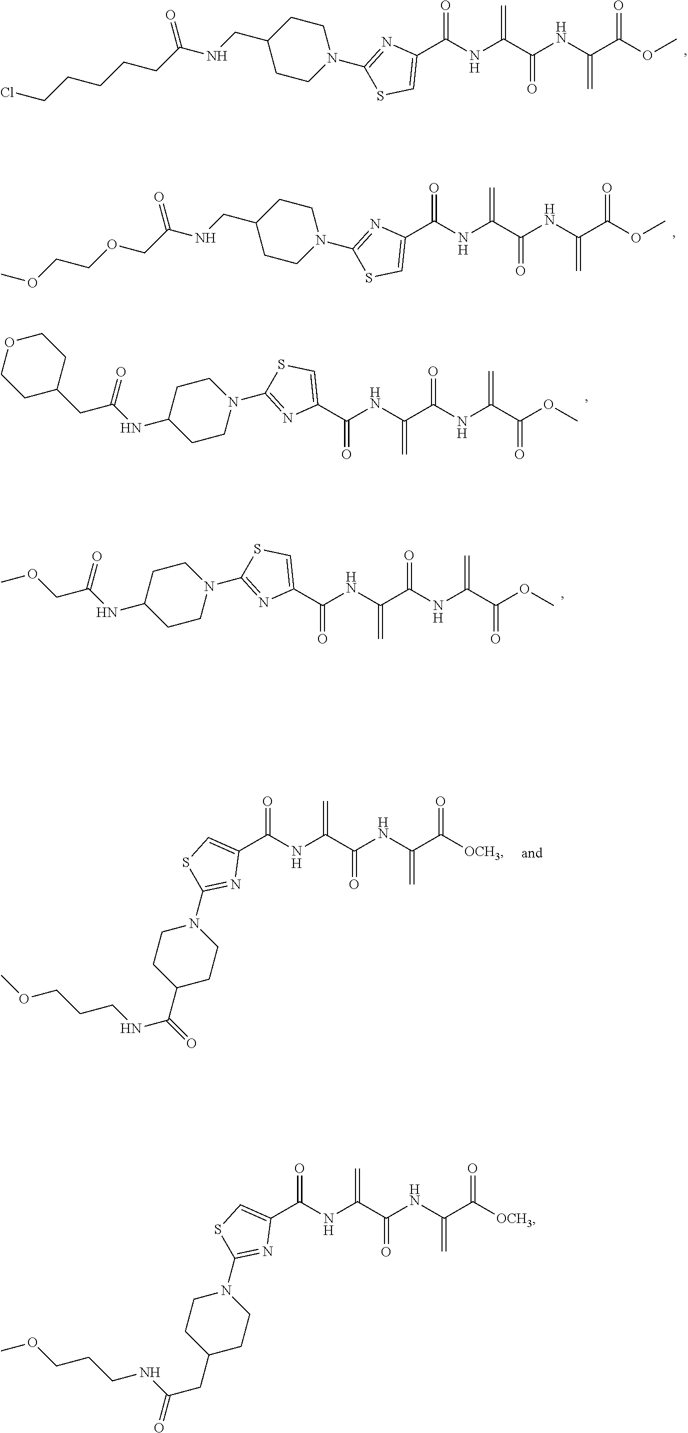
or a pharmaceutically acceptable salt thereof.

13. The compound of claim 1, wherein the compound is selected from the group consisting of: 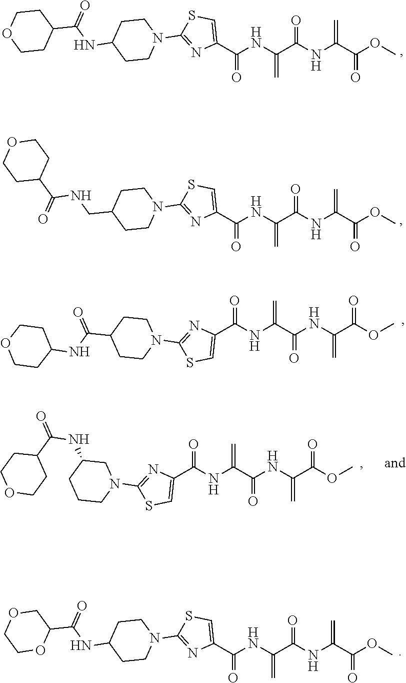 [SMILES: C=C(NC(=O)c1csc(N2CCC(NC(=O)C3CCOCC3)CC2)n1)C(=O)NC(=C)C(=O)OC ; C=C(NC(=O)c1csc(N2CCC(CNC(=O)C3CCOCC3)CC2)n1)C(=O)NC(=C)C(=O)OC ; C=C(NC(=O)c1csc(N2CCC(C(=O)NC3CCOCC3)CC2)n1)C(=O)NC(=C)C(=O)OC ; C=C(NC(=O)c1csc(N2CCC[C@H](NC(=O)C3CCOCC3)C2)n1)C(=O)NC(=C)C(=O)OC ; C=C(NC(=O)c1csc(N2CCC(NC(=O)C3COCCO3)CC2)n1)C(=O)NC(=C)C(=O)OC]
or a pharmaceutically acceptable salt thereof.

14. The compound of claim 1, wherein R¹ is a group having a structure of: 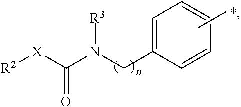
wherein:
n is 0, 1, 2 or 3;
X is O or CH₂;
R² is alkyl (e.g., having from 1 to 8 carbon atoms, linear or branched), wherein said alkyl is optionally substituted (e.g., with halo, amino, ether, alkoxy, or carbamate), or heterocycle; and
R³ is H or alkyl (e.g., having from 1 to 8 carbon atoms, linear or branched),
wherein * denotes the connection of the group in the compound of Formula I,
or a pharmaceutically acceptable salt thereof.

15. The compound of claim 1, wherein the compound is selected from the group consisting of: 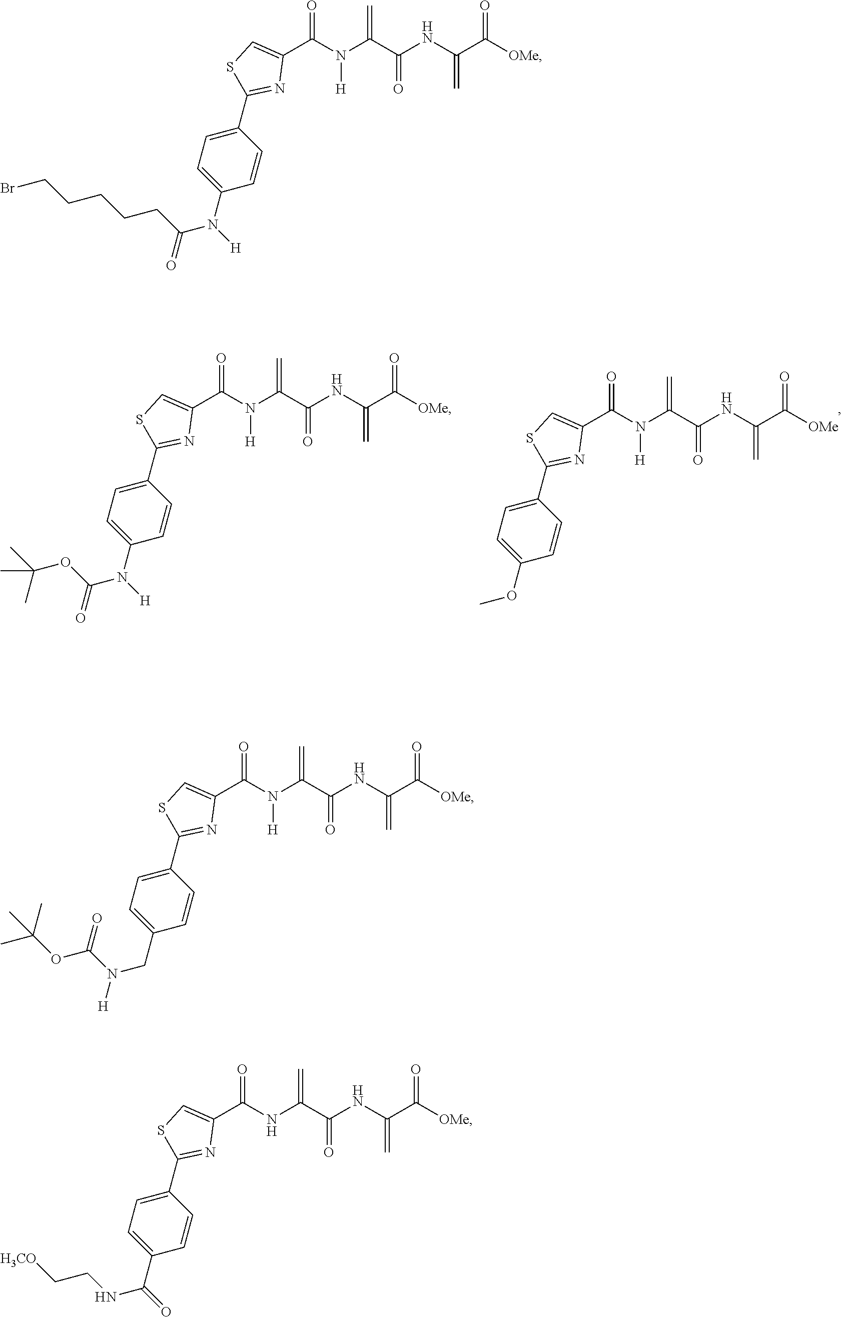 [SMILES: C=C(NC(=O)c1csc(-c2ccc(NC(=O)CCCCCBr)cc2)n1)C(=O)NC(=C)C(=O)OC ; C=C(NC(=O)c1csc(-c2ccc(NC(=O)OC(C)(C)C)cc2)n1)C(=O)NC(=C)C(=O)OC ; C=C(NC(=O)c1csc(-c2ccc(OC)cc2)n1)C(=O)NC(=C)C(=O)OC ; C=C(NC(=O)c1csc(-c2ccc(CNC(=O)OC(C)(C)C)cc2)n1)C(=O)NC(=C)C(=O)OC ; C=C(NC(=O)c1csc(-c2ccc(C(=O)NCCOC)cc2)n1)C(=O)NC(=C)C(=O)OC] 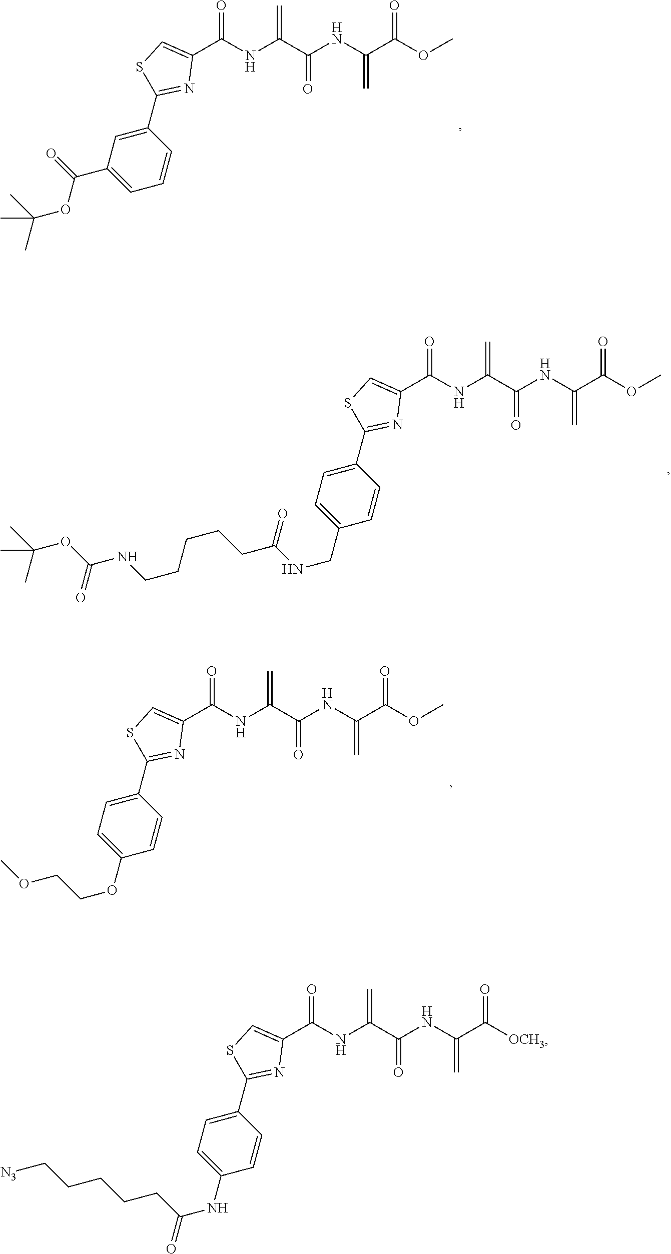 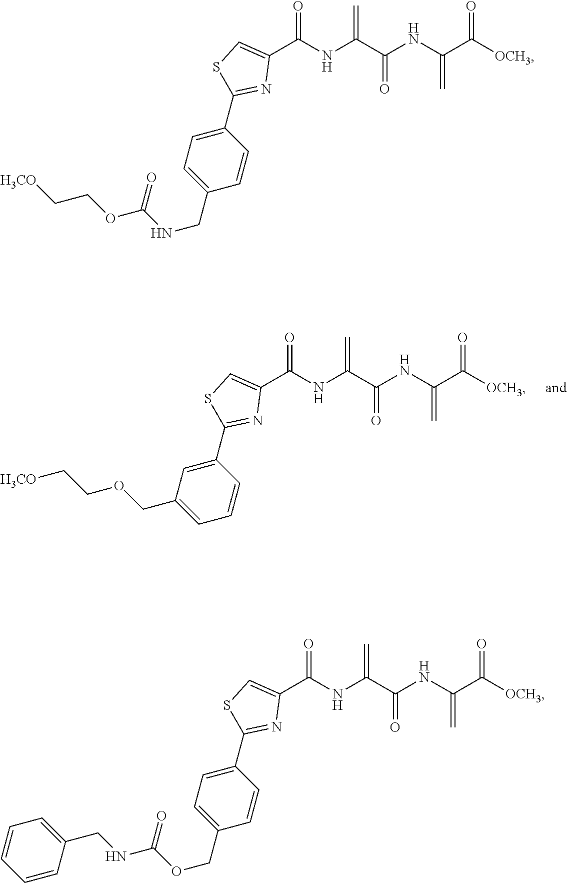 [SMILES: C=C(NC(=O)c1csc(-c2ccc(CNC(=O)OCCOC)cc2)n1)C(=O)NC(=C)C(=O)OC ; C=C(NC(=O)c1csc(-c2cccc(COCCOC)c2)n1)C(=O)NC(=C)C(=O)OC ; C=C(NC(=O)c1csc(-c2ccc(COC(=O)NCc3ccccc3)cc2)n1)C(=O)NC(=C)C(=O)OC]
or a pharmaceutically acceptable salt thereof.

16. The compound of claim 1, wherein the compound is selected from the group consisting of: 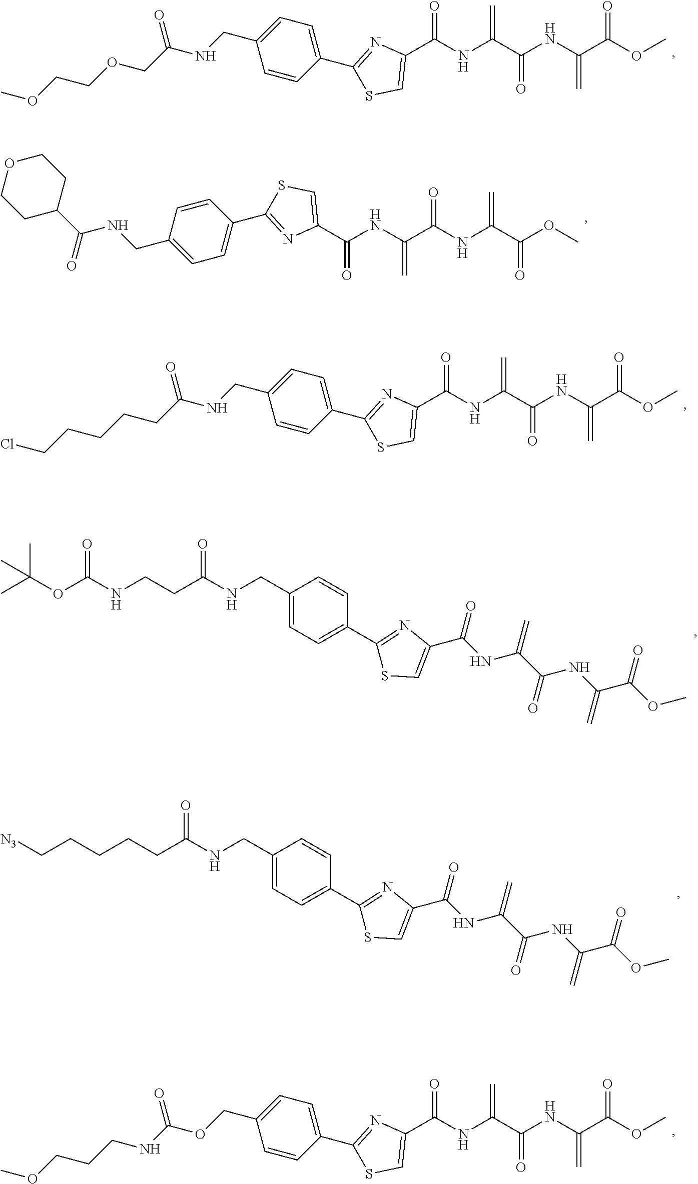 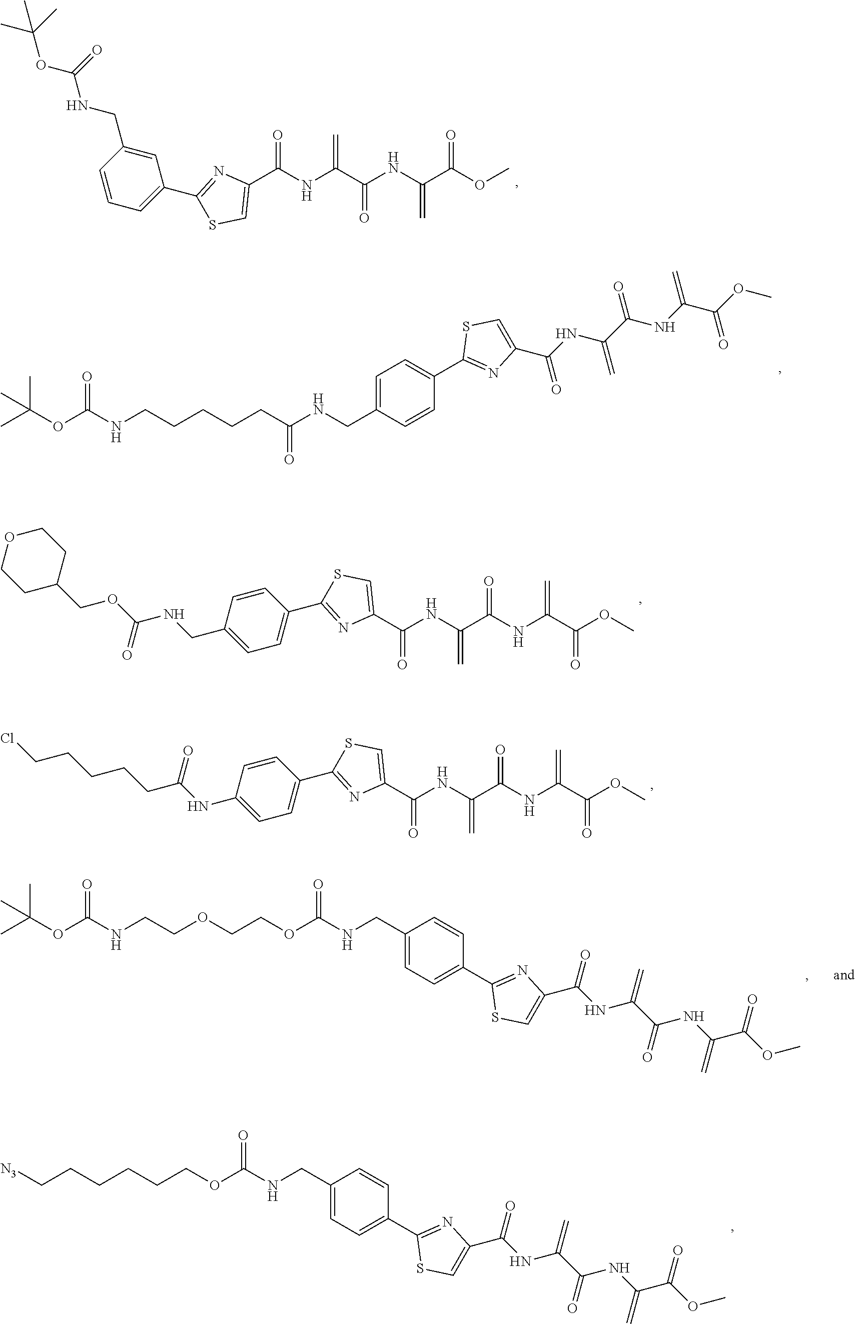
or a pharmaceutically acceptable salt thereof.

17. The compound of claim 1, wherein the compound is selected from the group consisting of: 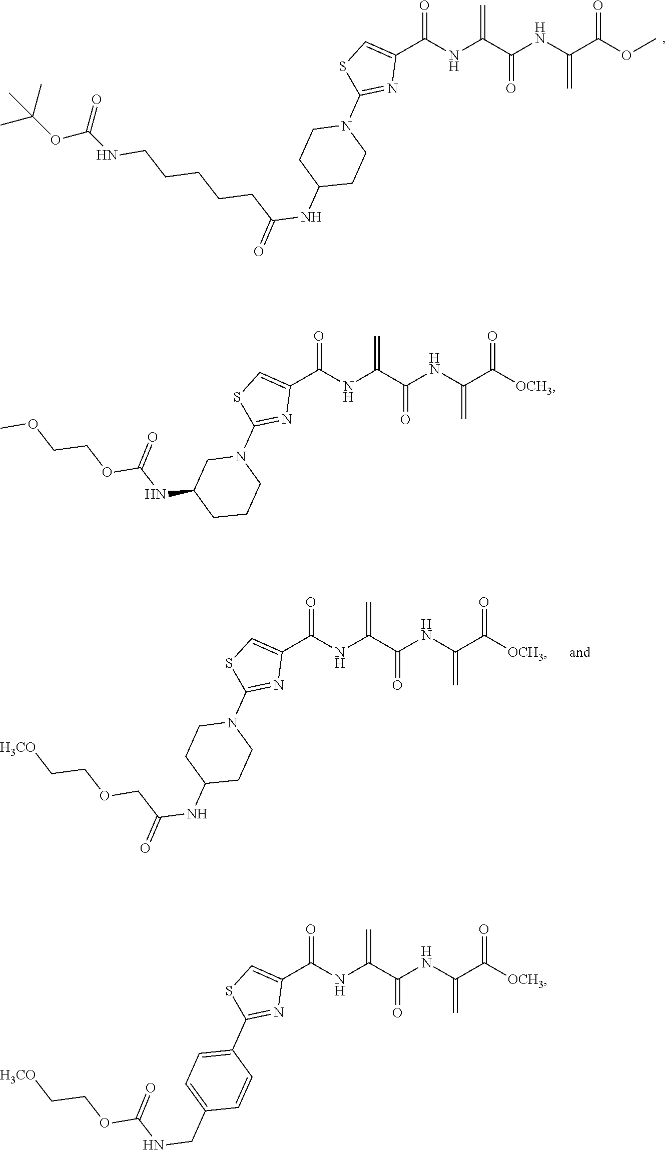
or a pharmaceutically acceptable salt thereof.

18. A pharmaceutical composition comprising a compound or pharmaceutically acceptable salt of claim 1.

19. The composition of claim 18, wherein said composition is formulated for oral or parenteral (e.g. intravenous, intrapleural, intraperitoneal or intraovarian) administration.

20. The composition of claim 18, wherein said composition is formulated for oral administration and is in the form of a capsule, cachet, lozenge, or tablet.

21. The composition of claim 18, wherein said formulation is provided in unit dosage form of from 1 mg to 10 grams of the compound, pharmaceutically acceptable salt or prodrug.

22. A method treating cancer in a subject in need thereof, comprising administering to the subject a therapeutically effective amount of a compound or pharmaceutically acceptable salt of claim 1.

23. The method of claim 22, wherein the cancer has PRX3 expression.

24. The method of claim 22, wherein said subject is a human subject.

25. The method of claim 22, wherein said subject is a non-human animal subject (e.g. non-human mammalian subject).

26. The method of claim 22, wherein said administering is carried out by administering a pharmaceutical composition comprising said compound or pharmaceutically acceptable salt.

27. The method of claim 22, wherein said administering further comprises administering bortezomib, carboplatin, paclitaxel, an immunotherapy agent, or a combination thereof.

28. The method of claim 22, wherein said administering further comprises administering doxorubicin.

29. A method of inhibiting PRX3 in a subject in need thereof, comprising administering to said subject a therapeutically effective amount of a compound or pharmaceutically acceptable salt of claim 1.
